(19) United States
(12) Patent Application Publication
(10) Pub. No.: US 2024/0226106 A1
(43) Pub. Date: Jul. 11, 2024

(54) INHIBITORS OF PLASMA KALLIKREIN

(71) Applicant: SHIRE HUMAN GENETIC THERAPIES, INC., Lexington, MA (US)

(72) Inventors: Nikolaos PAPAIOANNOU, Lexington, MA (US); Jeremy Mark TRAVINS, Lexington, MA (US); Sarah Jocelyn FINK, Lexington, MA (US); John Mark ELLARD, Saffron Walden (GB); Alastair RAE, Saffron Walden (GB)

(21) Appl. No.: 18/550,504

(22) PCT Filed: Mar. 16, 2022
(86) PCT No.: PCT/US2022/020491

§ 371 Date: Sep. 14, 2023

Related U.S. Application Data
(60) Provisional application No. 63/162,487, filed on Mar. 17, 2021.

(51) Int. Cl. A61K 31/53; A61K 31/4155; A61K 31/437; A61K 31/4709; A61K 31/496; A61K 31/501; A61K 31/502; A61K 31/506; A61K 31/519; A61P 7/00; A61P 29/00; C07D 401/06; C07D 401/12; C07D 401/14; C07D 403/04; C07D 403/10; C07D 403/12; C07D 403/14; C07D 417/12; C07D 471/04; C07D 487/04; C07D 495/04; C07D 519/00

(52) U.S. Cl. CPC A61K 31/53; A61K 31/4155; A61K 31/437; A61K 31/4709; A61K 31/496; A61K 31/501; A61K 31/502; A61K 31/506; A61K 31/519; A61P 7/00; A61P 29/00; C07D 401/06; C07D 401/12; C07D 401/14; C07D 403/04; C07D 403/10; C07D 403/12; C07D 403/14; C07D 417/12; C07D 471/04; C07D 487/04; C07D 495/04; C07D 519/00

(57) ABSTRACT

The present invention provides compounds and compositions thereof which are useful as inhibitors of plasma kallikrein and which exhibit desirable characteristics for the same.

### CROSS REFERENCE TO RELATED APPLICATIONS

This application claims the benefit of U.S. Provisional Application No. 63/162,487, filed Mar. 17, 2021, which is herein incorporated by reference in its entirety.

### I. BACKGROUND OF THE INVENTION

Plasma Kallikrein (PKa) is a serine protease zymogen in blood that is converted to its catalytically active form by coagulation factor XIIa, and contributes to the innate inflammatory response and intrinsic cascade of blood coagulation. The mechanisms that lead to the activation of this pathway in vivo include interactions with polyphosphates released from activated platelets and deficiency of C1 inhibitor (C1-INH), the primary physiological inhibitor of PKa. PKa-mediated cleavage of high-molecular weight kininogen generates the potent vasodilator and pro-inflammatory nonapeptide bradykinin (BK), which activates the bradykinin 2 receptor. Subsequent cleavage of BK by carboxypeptidases generates des-Arg9-BK, which activates the B1 receptor. Both B1 and B2 receptors are expressed by vascular, glial, and neuronal cell types, with the highest levels of retinal expression detected in the ganglion cell layer and inner and outer nuclear layers. Activation of B1 and B2 receptors causes vasodilation and increases vascular permeability.

PKa is also associated with a number of disorders, such as hereditary angioedema (HAE), an autosomal dominant disease characterized by painful, unpredictable, recurrent attacks of inflammation affecting the hands, feet, face, abdomen, urogenital tract, and the larynx. Prevalence for HAE is uncertain but is estimated to be approximately 1 case per 50,000 persons without known differences among ethnic groups. HAE is caused by deficient (Type I) or dysfunctional (Type II) levels of C1-INH, which inhibits PKa, bradykinin, and other serine proteases in the blood. Individuals with hereditary angioedema (HAE) are deficient in C1-INH and consequently undergo excessive bradykinin generation, which in turn cause painful, debilitating, and potentially fatal swelling attacks. If left untreated, HAE can result in a mortality rate as high as 40% primarily due to upper airway obstruction.

### II. SUMMARY OF THE INVENTION

The present disclosure is based on, at least in part, the development of a number of compounds which bind to plasma kallikrein and effectively inhibit its activity. Accordingly, provided herein are compounds and uses thereof for targeting plasma kallikrein and/or treating plasma kallikrein-mediated diseases and disorders.

In some embodiments, the present invention provides a compound of Formula (I):

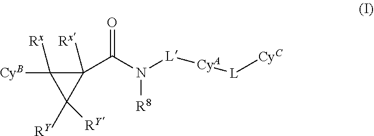

or a pharmaceutically acceptable salt thereof, wherein each of Cy^{A}, Cy^{B}, Cy^{C}, L, L′, R^{x}, R^{x′}, R^{Y}, R^{Y′}, and R⁸ is defined and described in classes and subclasses herein, both singly and in combination. In certain embodiments, the present invention provides compounds of Formulae (I)-(VI-b), as defined and described in classes and subclasses herein. In certain embodiments, the present invention provides novel intermediates and processes for preparing compounds disclosed herein. The disclosure also extends to pharmaceutical compositions comprising any one of the same, and use of compounds or compositions herein for treatment, in particular treatment of autoimmune disease, such as HAE or diabetic macular edema.

In some embodiments, the present invention also provides methods of using compounds of Formulae (I)-(VI-b).

Advantageously, the compounds of the present disclosure have therapeutic activity and/or adequate levels of bioavailability and/or adequate half-life for use as a therapeutic.

### III. DETAILED DESCRIPTION OF CERTAIN EMBODIMENTS

### A. Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The abbreviations used herein have their conventional meaning within the chemical and biological arts. The chemical structures and formulae set forth herein are constructed according to the standard rules of chemical valency known in the chemical arts.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocyclyl,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In some embodiments, aliphatic groups contain 1-4 aliphatic carbon atoms. In some embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocyclyl” or “cycloalkyl”) refers to a monocyclic C₃-C₇ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR* (as in N-substituted pyrrolidinyl)).

The term “unsaturated,” as used herein, means that a moiety has one or more units of unsaturation.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “halogen” means F, Cl, Br, or I.

The term “aryl” refers to monocyclic and bicyclic ring systems having a total of five to 10 ring members, wherein at least one ring in the system is aromatic and wherein each ring in the system contains three to seven ring members. The term “aryl” may be used interchangeably with the term “aryl ring”. In some embodiments, an 8-10 membered bicyclic aryl group is an optionally substituted naphthyl ring. In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-” refer to groups having 5 to 10 ring atoms, preferably 5, 6, or 9 ring atoms; having 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring (or in the case of a bivalent fused heteroarylene ring system, at least one radical or point of attachment is on a heteroaromatic ring). Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3 (4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted.

As used herein, the terms “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in this context in reference to a ring atom, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or +NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As used herein and unless otherwise specified, the suffix “-ene” is used to describe a bivalent group. Thus, any of the terms above can be modified with the suffix “-ene” to describe a bivalent version of that moiety. For example, a bivalent carbocycle is “carbocycylene”, a bivalent aryl ring is “arylene”, a bivalent benzene ring is “phenylene”, a bivalent heterocycle is “heterocyclylene”, a bivalent heteroaryl ring is “heteroarylene”, a bivalent alkyl chain is “alkylene”, a bivalent alkenyl chain is “alkenylene”, a bivalent alkynyl chain is “alkynylene”, and so forth.

As described herein, compounds of the invention may, when specified, contain “optionally substituted” moieties. In general, the term “substituted,” whether preceded by the term “optionally” or not, means that one or more hydrogens of the designated moiety are replaced with a suitable substituent. “Substituted” applies to one or more hydrogens that are either explicit or implicit from the structure (e.g.,

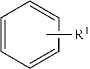

refers to at least

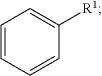

and

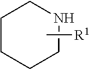

refers to at least

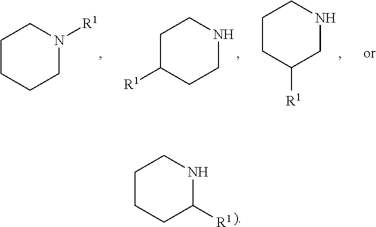

In addition, in a polycyclic ring system, substituents may, unless otherwise indicated, replace a hydrogen on any individual ring (e.g.,

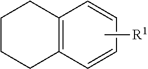

refers to at least

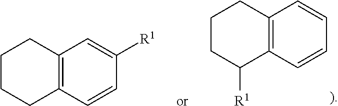

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O(CH₂)₀₋₄C(O)OR^{∘}; —O(CH₂)₀₋₄OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)_{0- 4}C(O)OSiR^{∘}₃; —(CH₂)₀₋₄OC(O)R^{∘}; —OC(O)(CH₂)₀₋₄SR^{∘}, —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6 membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12 membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{•} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR^{#}₂, ═NNHC(O)R^{#}, ═NNHC(O)OR^{#}, ═NNHS(O)₂R^{#}, ═NR^{#}, ═NOR^{#}, —O(C(R^{∩}₂))₂₋₃O—, or —S(C(R^{#}₂))₂₋₃S—, wherein each independent occurrence of R⁴ is selected from hydrogen, C₁₋₆ aliphatic which may be substituted as defined below, or an unsubstituted 5-6 membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR^{#}₂)₂₋₃O—, wherein each independent occurrence of R^{#} is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{#} include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R* is unsubstituted or where preceded by “halo” is substituted only with one or more halogens, and is independently C₁₋₄ aliphatic, —CH₂Ph, —O(CH₂)₀₋₁Ph, or a 5-6 membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R, taken together with their intervening atom(s) form an unsubstituted 3-12 membered saturated, partially unsaturated, or aryl mono- or bicyclic ring having 0-4 heteroatoms independently selected from nitrogen, oxygen, or sulfur.

Suitable substituents on the aliphatic group of R are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference.

In certain embodiments, the neutral forms of the compounds are regenerated by contacting the salt with a base or acid and isolating the parent compound in the conventional manner. In some embodiments, the parent form of the compound differs from the various salt forms in certain physical properties, such as solubility in polar solvents.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

In some embodiments, compounds of the present disclosure are provided as a single enantiomer or single diastereoisomer. Single enantiomer refers to an enantiomeric excess of 80% or more, such as 81, 82, 83, 84, 85, 86, 87, 88, 89, 90, 91, 92, 93, 94, 95, 96, 97, 98 or 99%. Single diastereoisomer excess refers to an excess of 80% or more, for example 81, 82, 83, 84, 85, 86, 87, 88, 89, 90, 91, 92, 93, 94, 95, 96, 97, 98 or 99%.

The term “oxo,” as used herein, means an oxygen that is double bonded to a carbon atom, thereby forming a carbonyl.

The symbol “ [symbol] ”, except when used as a bond to depict unknown or mixed stereochemistry, denotes the point of attachment of a chemical moiety to the remainder of a molecule or chemical formula.

The articles “a” and “an” are used herein to refer to one or to more than one (i.e., to at least one) of the grammatical object of the article. By way of example, “an element” means one element or more than one element.

A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses.

As will be understood from context, a “reference” compound is one that is sufficiently similar to a particular compound of interest to permit a relevant comparison. In some embodiments, information about a reference compound is obtained simultaneously with information about a particular compound. In some embodiments, information about a reference compound is historical. In some embodiments, information about a reference compound is stored, for example in a computer-readable medium. In some embodiments, comparison of a particular compound of interest with a reference compound establishes identity with, similarity to, or difference of the particular compound of interest relative to the compound.

As used herein, the phrase “therapeutic agent” refers to any agent that has a therapeutic effect and/or elicits a desired biological and/or pharmacological effect, when administered to a subject.

As used herein, the term “therapeutically effective amount” refers to an amount of a therapeutic agent that confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic agent effective to treat, ameliorate, or prevent a desired disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount is commonly administered in a dosing regimen that may comprise multiple unit doses. For any particular therapeutic agent, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular subject may depend upon a variety of factors including the disorder being treated and the severity of the disorder; the activity of the specific therapeutic agent employed; the specific composition employed; the age, body weight, general health, sex and diet of the subject; the time of administration, route of administration, and/or rate of excretion or metabolism of the specific therapeutic agent employed; the duration of the treatment; and like factors as is well known in the medical arts.

As used herein, the term “treatment” (also “treat” or “treating”) refers to any administration of a substance (e.g., provided compositions) that partially or completely alleviates, ameliorates, relives, inhibits, delays onset of, reduces severity of, and/or reduces incidence of one or more symptoms, features, and/or causes of a particular disease, disorder, and/or condition. Such treatment may be of a subject who does not exhibit signs of the relevant disease, disorder and/or condition and/or of a subject who exhibits only early signs of the disease, disorder, and/or condition. Alternatively, or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

### B. Compounds

In some embodiments, a provided compound is of Formula (I):

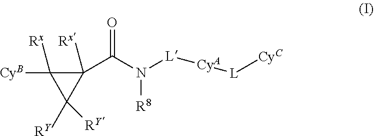

or a pharmaceutically acceptable salt thereof,
wherein:
Cy^{A} is a phenylene, a 5- to 6-membered monocyclic heteroarylene having 1-3 heteroatoms independently selected from oxygen, nitrogen, and sulfur, or a 7- to 12-membered bicyclic heteroarylene having 1-4 heteroatoms independently selected from oxygen, nitrogen, and sulfur, wherein Cy^{A} is substituted with 0-4 -R^{A} groups;
   each R^{A} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, phenyl, 3- to 7-membered saturated or partially unsaturated monocyclic carbocyclyl, or 3- to 7-membered saturated or partially unsaturated monocyclic heterocyclyl having 1-2 heteroatoms selected from oxygen, nitrogen, or sulfur;
each R is independently hydrogen or an optionally substituted C₁₋₆ aliphatic group;
each R^{Y} and R^{Y′} is independently selected from hydrogen, halogen, and an optionally substituted C₁₋₆ aliphatic group;
each R^{x} and R^{x′} is independently selected from hydrogen, halogen, or —CN;
Cy^{B} is selected from phenyl, 8- to 10-membered bicyclic aryl, a 5- to 6-membered heteroaryl having 1-3 heteroatoms independently selected from oxygen, nitrogen, and sulfur or a 7- to 10-membered heteroaryl having 1-4 heteroatoms independently selected from oxygen, nitrogen, and sulfur, wherein Cy^{B} is substituted with 0-4 -R^{B} groups; or
Cy^{B} and R^{x}, together with their intervening atoms, form a 6- to 12-membered spirocyclic ring system having 0-4 heteroatoms independently selected from oxygen, nitrogen, and sulfur, wherein the ring or rings formed by Cy^{B} and R^{x} may be substituted with 0-4 -R^{B} groups;
   each R^{B} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, a 3- to 7-membered saturated or partially unsaturated monocyclic heterocyclyl having 1-2 heteroatoms selected from oxygen, nitrogen, or sulfur, or a 5- to 6-membered heteroaryl having 1-4 heteroatoms independently selected from oxygen, nitrogen, and sulfur;
L is an optionally substituted C₁₋₃ hydrocarbon chain, wherein 1 to 3 methylene units are optionally and independently replaced with —C(O)—, —O—, —NR^{z}—, —S—, —SO—, —SO₂—, an optionally substituted cyclopropylene, or an optionally substituted 5- to 6-membered saturated or partially unsaturated heterocyclene, having 1-3 heteroatoms independently selected from oxygen, nitrogen, and sulfur;
   each R^{z} is independently selected from hydrogen, —(CH₂)₀₋₃OR, —(CH₂)₀₋₃C(O)OR, or an optionally substituted C₁₋₆ aliphatic group;
L′ is a covalent bond or an optionally substituted C₁₋₃ hydrocarbon chain, wherein 1 to 3 methylene units are optionally and independently replaced with —O—, —NR^{z}—, —S—, —SO—, or SO₂—;
Cy^{C} is selected from a 5- to 6-membered heteroaryl having 1-3 heteroatoms independently selected from oxygen, nitrogen, and sulfur, phenyl, 8- to 10-membered bicyclic aryl, a 7- to 10-membered heteroaryl having 1-4 heteroatoms independently selected from oxygen, nitrogen, and sulfur, or a 6- to 12-membered saturated or partially unsaturated fused bicyclic heterocyclyl having 1-4 heteroatoms independently selected from oxygen, nitrogen, or sulfur, wherein Cy^{C} is substituted with 0-6 -L^{C}-R^{C} groups;
   each L^{C} is independently selected from a covalent bond or an optionally substituted C₁₋₆ hydrocarbon chain, wherein 1 to 3 methylene units are optionally and independently replaced with —O— or —NR—;
   each R^{C} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, phenyl, a 3- to 7-membered saturated or partially unsaturated monocyclic carbocyclyl, a 5- or 6-membered heteroaryl having 1-2 heteroatoms selected from oxygen, nitrogen, or sulfur; a 3- to 7-membered saturated or partially unsaturated monocyclic heterocyclyl having 1-2 heteroatoms selected from oxygen, nitrogen, or sulfur; a 6- to 12-membered saturated or unsaturated bicyclic heterocyclyl having 1-3 heteroatoms selected from oxygen, nitrogen, or sulfur; and
R⁸ is selected from hydrogen, —OR, or an optionally substituted C₁₋₆ aliphatic group.

In some embodiments, a provided compound is of Formula (I), provided that Cy^{C} is not

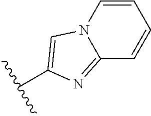 [SMILES: *c1cn2ccccc2n1]

substituted with 0-6 -L^{C}-R^{C} groups.

It will be appreciated that, “oxo” refers a double bonded oxygen substitution on a carbon “C═O”, where the carbon atom is part of the structure or group that is substituted by oxo. For example, where Cy^{C} is substituted with -L^{D}-R^{D}, and where L^{D} is a covalent bond and R^{D} is oxo, the carbon atom substituted with oxo (i.e., the carbon in C═O) is part of Cy^{C} (e.g., a structure of Cy^{C} being cyclopentyl substituted with -L^{D}-R^{D} at the 2-position, where L^{D} is a covalent bond and R^{D} is oxo corresponds to

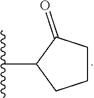 [SMILES: *C1CCCC1=O]

In some embodiments, Cy^{A} is a phenylene or a 5- to 6-membered monocyclic heteroarylene having 1-3 heteroatoms independently selected from oxygen, nitrogen, and sulfur, wherein Cy^{A} is substituted with 0-4 R^{A} groups. In some embodiments, Cy^{A} is a 5- to 6-membered monocyclic heteroarylene having 1-3 heteroatoms independently selected from oxygen, nitrogen, and sulfur, or a 7- to 12-membered bicyclic heteroarylene having 1-4 heteroatoms independently selected from oxygen, nitrogen, and sulfur, wherein Cy^{A} is substituted with 0-4 -R^{A} groups.

In some embodiments, Cy^{A} is a phenylene, wherein Cy^{A} is substituted with 0-4 -R^{A} groups. In some embodiments, Cy^{A} is a phenylene, wherein Cy^{A} is substituted with 0-2 -R^{A} groups.

In some embodiments, Cy^{A} is a 5- to 6-membered monocyclic heteroarylene having 1-3 heteroatoms independently selected from oxygen, nitrogen, and sulfur, wherein Cy^{A} is substituted with 0-4 -R^{A} groups. In some embodiments, Cy^{A} is a 6-membered monocyclic heteroarylene having 1-3 heteroatoms independently selected from oxygen, nitrogen, and sulfur, wherein Cy^{A} is substituted with 0-4 -R^{A} groups. In some embodiments, Cy^{A} is a 6-membered monocyclic heteroarylene having 1-3 nitrogen heteroatoms, wherein Cy^{A} is substituted with 0-4 R^{A} groups. In some embodiments, Cy^{A} is a pyridinediyl substituted with 0-3 R^{A} groups. In some embodiments, Cy^{A} is a pyrimidinediyl substituted with 0-2 R^{A} groups. In some embodiments, Cy^{A} is a pyridazinediyl substituted with 0-2 R^{A} groups. In some embodiments, Cy^{A} is a triazinediyl substituted with 0-1 R^{A} groups.

In some embodiments, Cy^{A} is a 5-membered monocyclic heteroarylene having 1-3 heteroatoms independently selected from oxygen, nitrogen, and sulfur, wherein Cy^{A} is substituted with 0-2 -R^{A} groups. In some embodiments, Cy^{A} is an unsubstituted thiadiazolediyl. In some embodiments, Cy is an unsubstituted oxadiazolediyl. In some embodiments, Cy is an unsubstituted triazolediyl.

In some embodiments, Cy^{A} is pyrazolediyl, wherein Cy^{A} is substituted with 0-2 -R^{A} groups. In some embodiments, Cy^{A} is imidazolediyl, wherein Cy^{A} is substituted with 0-2 -R^{A} groups. In some embodiments, Cy^{A} is unsubstituted pyrazolediyl. In some embodiments, Cy^{A} is unsubstituted imidazolediyl.

In some embodiments, Cy^{A} is a 7- to 12-membered bicyclic heteroarylene having 1-4 heteroatoms independently selected from oxygen, nitrogen, and sulfur, wherein Cy^{A} is substituted with 0-4 -R^{A} groups. In some embodiments, Cy^{A} is a 9-membered bicyclic heteroarylene having 3-4 heteroatoms independently selected from oxygen and nitrogen, wherein Cy^{A} is substituted with 0-1 -R^{A} groups. In some embodiments, Cy^{A} is a 10-membered bicyclic heteroarylene having 3-4 heteroatoms independently selected from oxygen and nitrogen, wherein Cy^{A} is substituted with 0-1 -R^{A} groups.

In some embodiments, Cy^{A} is selected from the group consisting of:

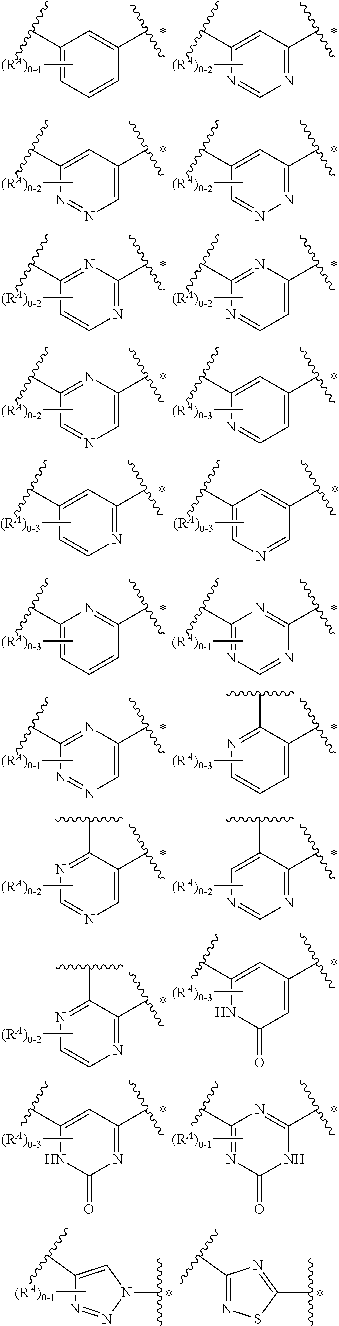

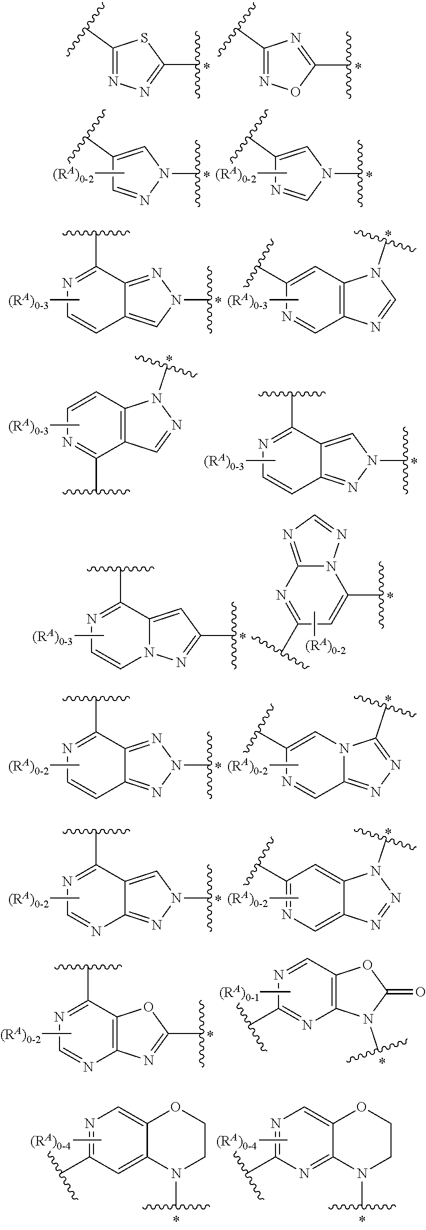

wherein * represents point of attachment to L.

In some embodiments, Cy^{A} is selected from the group consisting of:

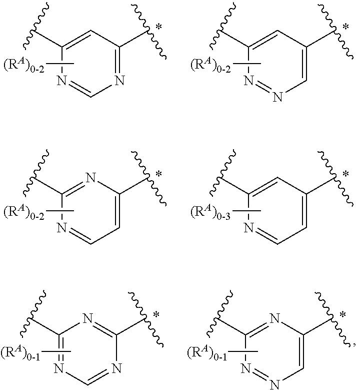

wherein * represents the point of attachment to L.

In some embodiments, Cy^{A} is selected from the group consisting of:

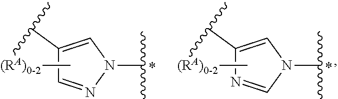

wherein * represents the point of attachment to L.

In some embodiments, Cy^{A} is selected from the group consisting of:

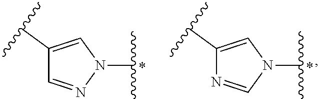 [SMILES: *c1cnn(*)c1 ; *c1cn(*)cn1]

wherein * represents the point of attachment to L.

In some embodiments, Cy^{A} is selected from the group consisting of:

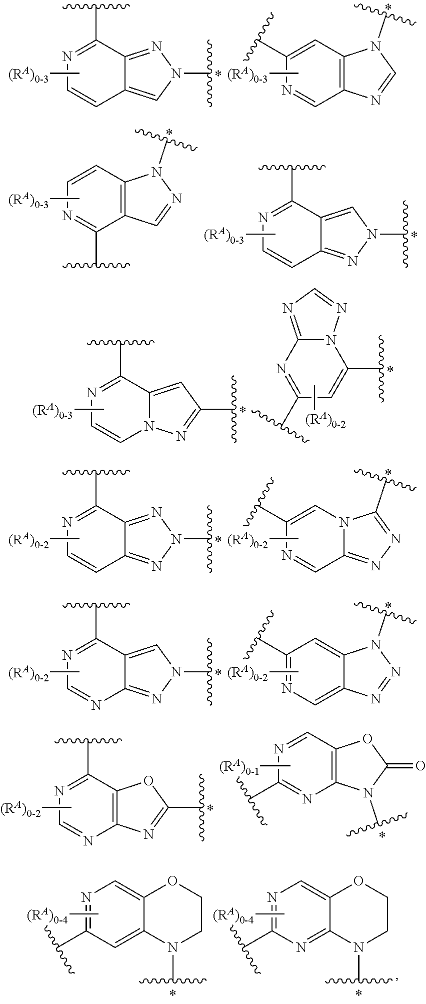

wherein * represents the point of attachment to L.

In some embodiments, Cy^{A} is

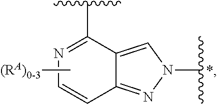

wherein * represents the point of attachment to L.

In some embodiments, Cy^{A} is selected from the group consisting of:

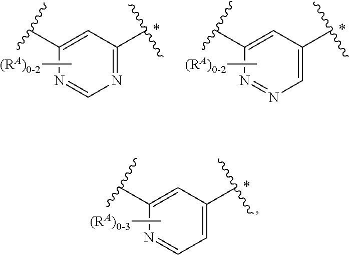

wherein * represents the point of attachment to L.

In some embodiments, Cy^{A} is:

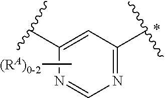

wherein * represents the point of attachment to L.

In some embodiments, Cy^{A} is:

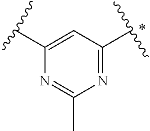 [SMILES: *c1cc(*)nc(C)n1]

wherein * represents the point of attachment to L.

In one embodiment, Cy^{A} comprising 0 R^{A} groups, i.e. Cy^{A} is unsubstituted.

In one embodiment, Cy^{A} comprises 1 R^{A} group, for example as described herein, in particular methyl.

In one embodiment, Cy^{A} comprises 2 R^{A} groups, for example independently selected from the groups/atoms described herein.

In some embodiments, each R^{A} is independently selected from oxo, halogen, —CN, —C(O)₂R, —N(R)₂, —OR, —SR, —S(O)R, —S(O)₂R, or an optionally substituted group selected from C₁₋₆ aliphatic, 3- to 7-membered saturated or partially unsaturated monocyclic carbocyclyl, or 3- to 7-membered saturated or partially unsaturated monocyclic heterocyclyl having 1-2 heteroatoms selected from oxygen, nitrogen, or sulfur.

In some embodiments, each R^{A} is independently selected from halogen, —OR, or an optionally substituted C₁₋₆ aliphatic. In some embodiments, each R^{A} is independently selected from halogen or an optionally substituted C₁₋₆ aliphatic. In some embodiments, each R^{A} is independently selected from fluorine or methyl.

In some embodiments, substituents on an optionally substituted R^{A} group are independently halogen, —(CH₂)₀₋₄OR^{∘}, or —(CH₂)₀₋₄N(R^{∘})₂, wherein each R^{∘} is independently as defined above and described in classes and subclasses herein.

It will be appreciated that references herein to embodiments in which “a single instance” of a substituent is defined are not limited to monosubstituted embodiments. For example, “[i]n some embodiments, a single instance of R^{A} is oxo” includes embodiments in which at least one instance of R^{A} is oxo and which may comprise one or more additional R^{A} groups as defined herein.

In some embodiments, a single instance of R^{A} is oxo. In some embodiments, a single instance of R^{A} is halogen. In some embodiments, a single instance of R^{A} is fluorine. In some embodiments, a single instance of R^{A} is chlorine. In some embodiments, a single instance of R^{A} is —CN. In some embodiments, a single instance of R^{A} is —C(O)₂R. In some embodiments, a single instance of R^{A} is —N(R)₂. In some embodiments, a single instance of R^{A} is —OR. In some embodiments, a single instance of R^{A} is —OR, wherein R is optionally substituted C₁₋₆ aliphatic. In some embodiments, a single instance of R^{A} is —OR, wherein R is C₁₋₆ aliphatic, optionally substituted with —(CH₂)₀₋₄R^{∘}, wherein R^{∘} is phenyl optionally substituted with —OR^{•}, wherein R^{•} is independently as defined above and described in classes and subclasses herein.

In some embodiments, a single instance of R^{A} is —SR. In some embodiments, a single instance of R^{A} is —SR, wherein R is optionally substituted C₁₋₆ aliphatic. In some embodiments, a single instance of R^{A} is —S(O)R. In some embodiments, a single instance of R^{A} is —S(O)R, wherein R is optionally substituted C₁₋₆ aliphatic. In some embodiments, a single instance of R^{A} is —S(O)₂R. In some embodiments, a single instance of R^{A} is —S(O)₂R, wherein R is optionally substituted C₁₋₆ aliphatic.

In some embodiments, a single instance of R^{A} is C₁₋₆ aliphatic substituted with halogen. In some embodiments, Ra is —CF₃. In some embodiments, a single instance of R^{A} is C₁₋₆ aliphatic substituted with —(CH₂)₀₋₄OR^{∘}, wherein R^{∘} is selected from hydrogen or C₁₋₆ aliphatic. In some embodiments, a single instance of R^{A} is C₁₋₆ aliphatic substituted with —(CH₂)₀₋₄N(R^{∘})₂, wherein each R^{∘} is independently selected from hydrogen or C₁₋₆ aliphatic.

In some embodiments, a single instance of R^{A} is methyl, ethyl, or propyl.

In some embodiments, a single instance of R^{A} is optionally substituted 3- to 7-membered saturated or partially unsaturated monocyclic carbocyclyl. In some embodiments, a single instance of R^{A} is optionally substituted cyclopropyl.

In some embodiments, a single instance of R^{A} is optionally substituted 3- to 7-membered saturated or partially unsaturated monocyclic heterocyclyl having 1-2 heteroatoms selected from oxygen, nitrogen, or sulfur. In some embodiments, a single instance of R^{A} is optionally substituted 3- to 7-membered saturated monocyclic heterocyclyl having 1-2 heteroatoms selected from oxygen and nitrogen. In some embodiments, a single instance of R^{A} is optionally substituted oxetanyl. In some embodiments, a single instance of R^{A} is oxetanyl optionally substituted with halogen or —(CH₂)₀₋₄OR^{∘}. In some embodiments, a single instance of R^{A} is pyrrolidinyl.

In some embodiments, Cy^{B} is selected from phenyl, a 5- to 6-membered heteroaryl having 1-3 heteroatoms independently selected from oxygen, nitrogen, and sulfur, or a 7- to 10-membered heteroaryl having 1-4 heteroatoms independently selected from oxygen, nitrogen, and sulfur, wherein Cy^{B} is substituted with 0-4 -R^{B} groups.

In some embodiments, Cy^{B} is selected from phenyl or a 5- to 6-membered heteroaryl having 1-3 heteroatoms independently selected from oxygen, nitrogen, and sulfur, wherein Cy^{B} is substituted with 0-4 -R^{B} groups, for example pyrimidinyl substituted with 0-4 -R^{B} groups, such as 0 or 1 group (in particular wherein 1 group is methyl).

In some embodiments, Cy^{B} is selected from phenyl or a 5- to 6-membered heteroaryl having 1-3 heteroatoms independently selected from oxygen, nitrogen, and sulfur, wherein Cy^{B} is substituted with 0-4 -R^{B} groups.

In some embodiments, Cy^{B} is phenyl, wherein Cy^{B} is substituted with 0-4 -R^{B} groups. In some embodiments, Cy^{B} is phenyl, wherein Cy^{B} is substituted with 0-3 -R^{B} groups.

In some embodiments, Cy^{B} is a 6-membered heteroaryl having 1-3 heteroatoms independently selected from oxygen, nitrogen, and sulfur, wherein Cy^{B} is substituted with 0-4 -R^{B} groups. In some embodiments, Cy^{B} is a 6-membered heteroaryl having 1-3 nitrogens, wherein Cy^{B} is substituted with 0-4 -R^{B} groups. In some embodiments, Cy^{B} is a pyrimidinyl group substituted with 0-3 -R^{B} groups. In some embodiments, Cy^{B} is a pyridinyl group substituted with 0-4 -R^{B} groups. In some embodiments, Cy^{B} is a pyrimidinyl group substituted with 0-2 -R^{B} groups. In some embodiments, Cy^{B} is a pyridinyl group substituted with 0-2 -R^{B} groups. In some embodiments, Cy^{B} is a pyrazinyl group substituted with 0-1 -R^{B} groups. In some embodiments, Cy^{B} is a pyridazinyl group substituted with 0-1 -R^{B} groups. In some embodiments, Cy^{B} is a 1,3,5-triazinyl group substituted with 0-1 -R^{B} groups.

In some embodiments, Cy^{B} is a 5-membered heteroaryl having 1-3 heteroatoms independently selected from oxygen, nitrogen, and sulfur, wherein Cy^{B} is substituted with 0-4 -R^{B} groups. In some embodiments, Cy^{B} is a 5-membered heteroaryl having 1-2 heteroatoms independently selected from sulfur and nitrogen, wherein Cy^{B} is substituted with 0-4 -R^{B} groups. In some embodiments, Cy^{B} is a 5-membered heteroaryl having 1-2 nitrogens, wherein Cy^{B} is substituted with 0-4 -R^{B} groups. In some embodiments, Cy^{B} is pyrazolyl substituted with 0-3 -R^{B} groups. In some embodiments, Cy^{B} is a thienyl group substituted with 0-2 -R^{B} groups. In some embodiments, Cy^{B} is a thiazolyl group substituted with 0-1 -R^{B} groups. In some embodiments, Cy^{B} is a thiadiazolyl group substituted with 0-1 -R^{B} groups.

In some embodiments, Cy^{B} is selected from the group consisting of:

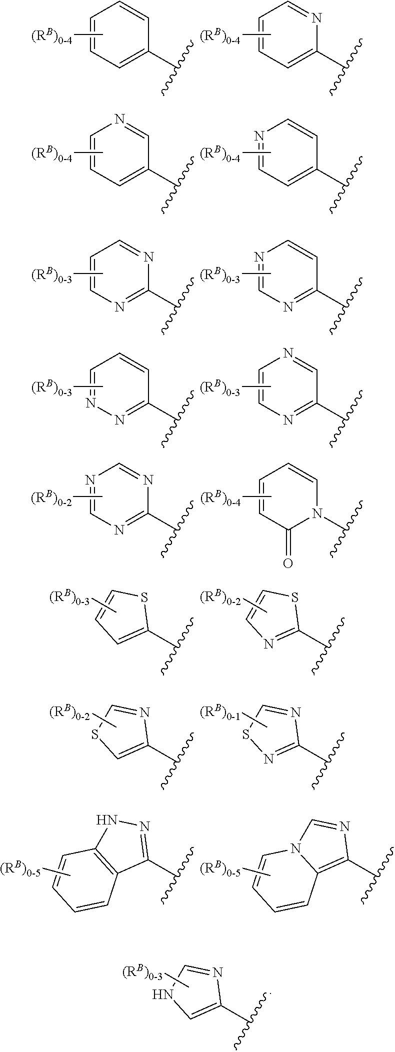

In some embodiments, Cy^{B} is selected from the group consisting of:

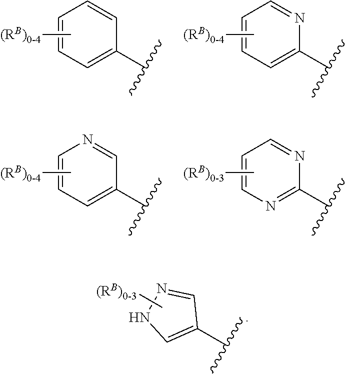

In some embodiments, Cy^{B} is selected from the group consisting of:

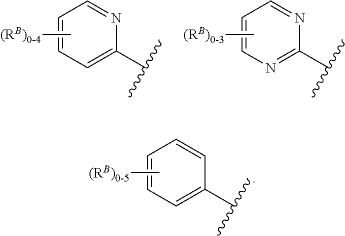

In some embodiments, Cy^{B} is:

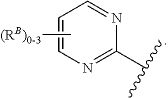

In some embodiments, Cy^{B} and R^{x}, together with their intervening atoms, form a 6- to 12-membered spirocyclic ring system having 0-4 heteroatoms independently selected from oxygen, nitrogen, and sulfur, wherein the ring or rings formed by Cy^{B} and R^{x} may be substituted with 0-4 -R^{B} groups. It will be appreciated that references herein to the number of atoms in a spirocyclic ring system (e.g., 6- to 12-membered) include the depicted cyclopropyl ring.

In some embodiments, Cy^{B} and R^{x}, together with their intervening atoms, form a 6- to 12-membered spirocyclic ring system having 0-1 nitrogen heteroatoms, wherein the ring or rings formed by Cy^{B} and R^{x} may be substituted with 1-3 -R^{B} groups.

In some embodiments, Cy^{B} and R^{x}, together with their intervening atoms, form a 6- to 12-membered spirocyclic ring system selected from:

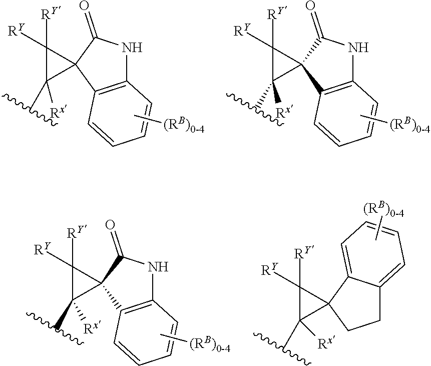

In some embodiments, each R^{B} is independently selected from oxo, halogen, —CN, —NO₂, —N(R)₂, —N(R)C(O)₂R, —OR, or an optionally substituted group selected from C₁₋₆ aliphatic or a 5-membered heteroaryl having 1-4 heteroatoms independently selected from oxygen, nitrogen, and sulfur.

In some embodiments, each R^{B} is independently selected from oxo, halogen, —CN, —N(R)₂, or an optionally substituted C₁₋₆ aliphatic.

In some embodiments, substituents on an optionally substituted B group are independently selected from oxo, halogen, and —(CH₂)₀₋₄OR^{∘}, wherein R^{∘} is as defined above and described in classes and subclasses herein.

In some embodiments, a single instance of R^{B} is oxo. In some embodiments, a single instance of R^{B} is halogen. In some embodiments, a single instance of R^{B} is fluorine. In some embodiments, a single instance of R^{B} is chlorine. In some embodiments, a single instance of R^{B} is —CN. In some embodiments, a single instance of R^{B} is —NO₂. In some embodiments, a single instance of R^{B} is —N(R)₂, In some embodiments, a single instance of R^{B} is —NH₂. In some embodiments, a single instance of R^{B} is —N(R)C(O)₂R. In some embodiments, a single instance of R^{B} is —OR. In some embodiments, a single instance of R^{B} is —OMe.

In some embodiments, a single instance of R^{B} is optionally substituted C₁₋₆ aliphatic. In some embodiments, a single instance of R^{B} is methyl, ethyl, or propyl. In some embodiments, a single instance of R^{B} is C₁₋₆ aliphatic substituted with halogen. In some embodiments, a single instance of R^{B} is —CF₃.

In some embodiments, a single instance of R^{B} is —N(R)C(O)₂R, wherein each R is independently selected from hydrogen or C₁₋₆ aliphatic optionally substituted with —(CH₂)₀₋₄R^{∘}.

In some embodiments, a single instance of R^{B} is —OR, wherein each R is independently selected from hydrogen or C₁₋₆ aliphatic optionally substituted with halogen, —(CH₂)₀₋₄OR^{∘}, or (CH₂)₀₋₄C(O)OR^{∘}.

In some embodiments, a single instance of R^{B} is a 5-membered heteroaryl having 1-4 heteroatoms independently selected from oxygen, nitrogen, and sulfur. In some embodiments, a single instance of R^{B} is tetrazolyl.

In some embodiments, each of R^{x} and R^{x′} is independently selected from hydrogen and halogen. In some embodiments, each of R^{x} and R^{x′} is hydrogen. In some embodiments, one of R^{x} and R^{x′} is hydrogen and the other is halogen.

In some embodiments, each of R^{Y} and R^{Y′} is independently selected from hydrogen and halogen. In some embodiments, each of R^{Y} and R^{Y′} is hydrogen. In some embodiments, R is an optionally substituted C₁₋₆ aliphatic group and R^{Y′} is hydrogen. In some embodiments, R is substituted with —(CH₂)₀₋₄OR^{∘}, wherein R^{∘} is as defined above and described in classes and subclasses herein.

In some embodiments, L is an optionally substituted C₁₋₃ hydrocarbon chain, wherein 1-3 methylene units are optionally and independently replaced with —O—, —NR—, —S—, or —SO₂—. In some embodiments, L is an optionally substituted C₁₋₃ hydrocarbon chain, wherein 1 methylene unit is optionally replaced with —O—, —NR—, —S—, or —SO₂—.

In some embodiments, L is —NR^{z}—.

In some embodiments, L is an optionally substituted C₁₋₃ hydrocarbon chain, wherein 1-3 methylene units are optionally and independently replaced with —C(O)—, —O—, —NR—, an optionally substituted cyclopropylene, or an optionally substituted 5- to 6-membered saturated or partially unsaturated heterocyclene, having 1-3 heteroatoms independently selected from oxygen, nitrogen, and sulfur. In some embodiments, L is an optionally substituted C₁₋₃ hydrocarbon chain, wherein 1-3 methylene units are optionally and independently replaced with —C(O)—, —O—, —NR—, cyclopropylene, or an optionally substituted 5-membered saturated or partially unsaturated heterocyclene, having 1-3 heteroatoms independently selected from oxygen, nitrogen, and sulfur.

In some embodiments, L is an optionally substituted C₁ hydrocarbon chain. In some embodiments, L is an optionally substituted C₁ hydrogen chain, wherein 1 methylene unit is optionally replaced with an optionally substituted cyclopropylene or an optionally substituted 5-membered saturated or partially unsaturated heterocyclene, having 1-3 heteroatoms independently selected from oxygen, nitrogen, and sulfur. In some embodiments, L is an optionally substituted C₁ hydrocarbon chain, wherein the 1 methylene unit is replaced with an optionally substituted cyclopropylene or an optionally substituted 5-membered saturated or partially unsaturated heterocyclene, having 1-3 heteroatoms independently selected from oxygen, nitrogen, and sulfur. In some embodiments, L is an optionally substituted C₁ hydrocarbon chain, wherein the 1 methylene unit is replaced with an optionally substituted cyclopropylene. In some embodiments, L is an optionally substituted C₁ hydrocarbon chain, wherein the 1 methylene unit is replaced with 5-membered saturated or partially unsaturated heterocyclene having 1 nitrogen heteroatom, optionally substituted with —(CH₂)₀₋₄OR^{∘}, wherein R^{∘} is as defined above and described in classes and subclasses herein.

In some embodiments, L is —CH₂—. In some embodiments, L is

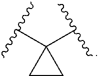 [SMILES: *C1(*)CC1]

In some embodiments, L is optionally substituted

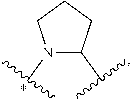 [SMILES: *C1CCCN1*]

wherein * represents the point of attachment to Cy^{A}. In some embodiments, L is optionally substituted

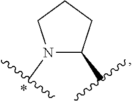 [SMILES: *[C@@H]1CCCN1*]

wherein * represents the point of attachment to Cy^{A}. In some embodiments, L is optionally substituted

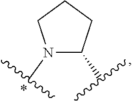 [SMILES: *[C@H]1CCCN1*]

wherein * represents the point of attachment to Cy^{A}. In some embodiments, L is

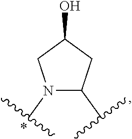 [SMILES: *C1C[C@H](O)CN1*]

wherein * represents the point of attachment to Cy^{A}. In some embodiments, L is

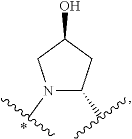 [SMILES: *[C@H]1C[C@H](O)CN1*]

wherein * represents the point of attachment to Cy^{A}. In some embodiments, L is

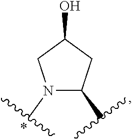 [SMILES: *[C@@H]1C[C@H](O)CN1*]

wherein * represents the point of attachment to Cy^{A}.

In some embodiments, L is an optionally substituted C2 hydrocarbon chain, wherein 1 methylene unit is optionally and independently replaced with —NR^{z}— or —O—. In some embodiments, L is —NR^{z}—. In some embodiments, L is an optionally substituted C2 hydrocarbon chain, wherein the methylene unit connected to Cy^{A} is replaced with —NR^{z}— or —O—. In some embodiments, L is an optionally substituted C₂ hydrocarbon chain, wherein the methylene unit connected to Cy^{A} is replaced with —NR^{z}—.

In some embodiments, R^{z} is selected from H and C₁₋₆ aliphatic group, such as H or methyl, in particular methyl.

In some embodiments, L is an optionally substituted C2 hydrocarbon chain, wherein the methylene unit connected to Cy^{A} is replaced with —O—. In some embodiments, L is *—NHCH(Me)-, wherein * represents the point of attachment to Cy^{A}. In some embodiments, L is

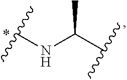 [SMILES: *N[C@H](*)C]

wherein * represents the point of attachment to Cy^{A}. In some embodiments, L is

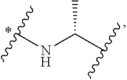 [SMILES: *N[C@@H](*)C]

wherein * represents the point of attachment to Cy^{A}. In some embodiments, L is *—NHCH₂—, wherein * represents the point of attachment to Cy^{A}. In some embodiments, L is *—N(CH₃)CH₂—, wherein* represents the point of attachment to Cy^{A}. In some embodiments, L is

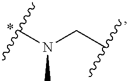

wherein * represents the point of attachment to Cy^{A}. In some embodiments, L is

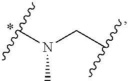

wherein * represents the point of attachment to Cy^{A}. In some embodiments, L is *—OCH(Me)-, wherein * represents the point of attachment to Cy^{A}. In some embodiments, L is *—OCH₂—, wherein * represents the point of attachment to Cy^{A}.

In some embodiments, L comprises a two-atom spacer between Cy^{A} and Cy^{C}.

In some embodiments, L is an optionally substituted C3 hydrocarbon chain, wherein 3 methylene units are optionally and independently replaced with —C(O)—, —NR—, or an optionally substituted 5-membered saturated or partially unsaturated heterocyclene, having 1-3 heteroatoms independently selected from oxygen, nitrogen, and sulfur. In some embodiments, L is an optionally substituted C₃ hydrocarbon chain, wherein 3 methylene units are independently replaced with —C(O)—, —NR^{z}—, or an optionally substituted 5-membered saturated or partially unsaturated heterocyclene, having 1 nitrogen heteroatom. In some embodiments, L is optionally substituted

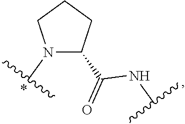 [SMILES: *NC(=O)[C@H]1CCCN1*]

wherein * represents the point of attachment to Cy^{A}. In some embodiments, L is

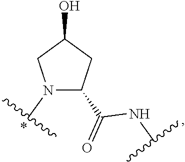 [SMILES: *NC(=O)[C@H]1C[C@H](O)CN1*]

wherein * represents the point of attachment to Cy^{A}. In some embodiments, L is

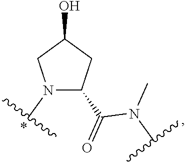 [SMILES: *N(C)C(=O)[C@H]1C[C@H](O)CN1*]

wherein * represents the point of attachment to Cy^{A}.

In some embodiments, optional substituents on L are independently selected from —(CH₂)₀₋₄R^{∘}, —(CH₂)₀₋₄OR^{∘}, —(CH₂)₀₋₄OC(O)R^{∘}, and —(CH₂)₀₋₄N(R^{∘})₂, wherein each R^{∘} is independently as defined above and described in classes and subclasses herein.

In some embodiments, L′ is a covalent bond or a methylene unit optionally substituted with —(CH₂)₀₋₄R^{∘}, wherein R^{∘} is independently as defined above and described in classes and subclasses herein. In some embodiments, R^{∘} is hydrogen or C₁₋₆ aliphatic.

In some embodiments, L′ is a covalent bond.

In some embodiments, R⁸ is hydrogen.

In some embodiments, R⁸ is selected from —OR or an optionally substituted C₁₋₆ aliphatic group.

In some embodiments, Cy^{C} is an 8- to 10-membered bicyclic aryl, wherein Cy^{C} is substituted with 0-6 -L^{C}-R^{C} groups. In some embodiments, Cy^{C} is quinolinyl, substituted with 0-6 -L^{C}-R^{C} groups.

In some embodiments, Cy^{C} is a 7- to 10-membered heteroaryl having 1-4 heteroatoms independently selected from oxygen, nitrogen, and sulfur, wherein Cy^{C} is substituted with 0-6 -L^{C}-R^{C} groups. In some embodiments, Cy^{C} is a 9- to 10-membered heteroaryl having 1-4 heteroatoms independently selected from oxygen, nitrogen, and sulfur, wherein Cy^{C} is substituted with 0-6 -L^{C}-R^{C} groups. In some embodiments, Cy^{C} is a 9-membered heteroaryl having 1-4 nitrogen heteroatoms, wherein Cy^{C} is substituted with 0-6 -L^{C}-R^{C} groups. In some embodiments, Cy^{C} is a 9-membered heteroaryl having 1 nitrogen and 1 sulfur heteroatoms, wherein Cy^{C} is substituted with 0-6 -L^{C}-R^{C} groups. In some embodiments, Cy^{C} is a 10-membered heteroaryl having 1-4 heteroatoms independently selected from oxygen, nitrogen, and sulfur, wherein Cy^{C} is substituted with 0-6 -L^{C}-R^{C} groups. In some embodiments, Cy^{C} is a 10-membered heteroaryl having 1 nitrogen heteroatom, wherein Cy^{C} is substituted with 0-6 -L^{C}-R^{C} groups.

In some embodiments, Cy^{C} is a 7- to 10-membered heteroaryl having 1-4 heteroatoms independently selected from oxygen, nitrogen, and sulfur, wherein Cy^{C} is substituted with 0-6 -L^{C}-R^{C} groups. In some embodiments, Cy^{C} is a 9-membered heteroaryl having 1-4 heteroatoms independently selected from oxygen, nitrogen, and sulfur, wherein Cy^{C} is substituted with 0-6 -L^{C}-R^{C} groups. In some embodiments, Cy^{C} is a 9-membered heteroaryl having 2 nitrogen heteroatoms, wherein Cy^{C} is substituted with 0-6 -L^{C}-R^{C} groups.

In some embodiments, Cy^{C} is triazolopyridinyl, wherein Cy^{C} is substituted with 0-4 -L^{C}-R^{C} groups. In some embodiments, Cy^{C} is pyrazolopyridinyl, wherein Cy^{C} is substituted with 0-5 -L^{C}-R^{C} groups. In some embodiments, Cy^{C} is pyrazolopyrimidinyl, wherein Cy^{C} is substituted with 0-4 -L^{C}-R^{C} groups. In some embodiments, Cy^{C} is triazolopyridazinyl, wherein Cy^{C} is substituted with 0-3 -L^{C}-R^{C} groups. In some embodiments, Cy^{C} is imidazopyridazinyl, wherein Cy^{C} is substituted with 0-4 -L^{C}-R^{C} groups. In some embodiments, Cy^{C} is imidazopyrimidinyl, wherein Cy^{C} is substituted with 0-4 -L^{C}-R^{C} groups. In some embodiments, Cy^{C} is imidazopyrimidinonyl, wherein Cy^{C} is substituted with 0-4 -L^{C}-R^{C} groups. In some embodiments, Cy^{C} is imidazopyrazinyl, wherein Cy^{C} is substituted with 0-4 -L^{C}-R^{C} groups. In some embodiments, Cy^{C} is benzoimidazolyl, wherein Cy^{C} is substituted with 0-4 -L^{C}-R^{C} groups. In some embodiments, Cy^{C} is triazolopyrimidinyl, wherein Cy^{C} is substituted with 0-3 -L^{C}-R^{C} groups. In some embodiments, Cy^{C} is thienopyridinyl, wherein Cy^{C} is substituted with 0-6 -L^{C}-R^{C} groups. In some embodiments, Cy^{C} is quinolinyl, wherein Cy^{C} is substituted with 0-6 -L^{C}-R^{C} groups.

In some embodiments, Cy^{C} is selected from the group consisting of:

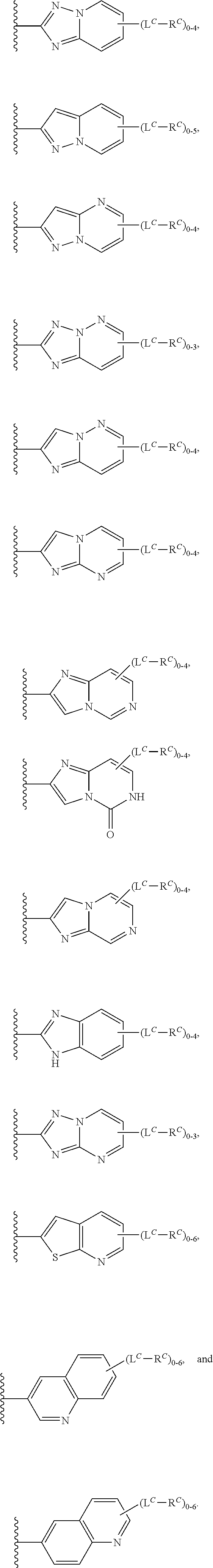

In some embodiments, Cy^{C} is selected from the group consisting of:

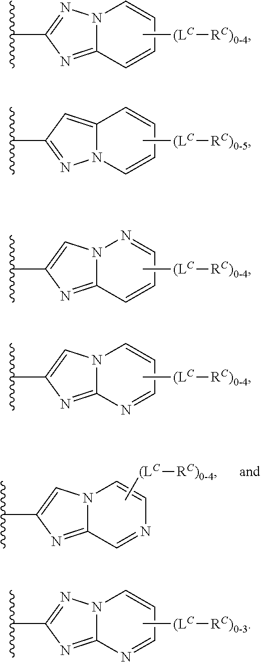

In some embodiments, Cy^{C} is selected from the group consisting of

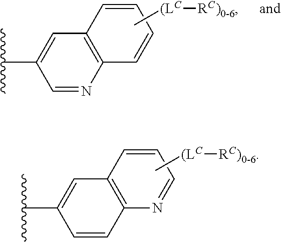

In some embodiments, Cy^{C} is phenyl, wherein Cy^{C} is substituted with 0-5 -L^{C}-R^{C} groups. In some embodiments, Cy^{C} is 5- to 6-membered heteroaryl having 1-3 heteroatoms independently selected from oxygen, nitrogen, and sulfur, wherein Cy^{C} is substituted with 0-6 -L^{C}-R^{C} groups. In some embodiments, Cy^{C} is 5-membered heteroaryl having 1-3 heteroatoms independently selected from oxygen, nitrogen, and sulfur, wherein Cy^{C} is substituted with 0-6 -L^{C}-R^{C} groups. In some embodiments, Cy^{C} is 5-membered heteroaryl having 1-2 heteroatoms independently selected from oxygen, nitrogen, and sulfur, wherein Cy^{C} is substituted with 0-6 -L^{C}-R^{C} groups. In some embodiments, Cy^{C} is 6-membered heteroaryl having 1-3 heteroatoms independently selected from oxygen, nitrogen, and sulfur, wherein Cy^{C} is substituted with 0-6 -L^{C}-R^{C} groups. In some embodiments, Cy^{C} is 6-membered heteroaryl having 1-3 nitrogen heteroatoms, wherein Cy^{C} is substituted with 0-6 -L^{C}-R^{C} groups.

In some embodiments, Cy^{C} is pyridineyl, wherein Cy^{C} is substituted with 0-5 -L^{C}-R^{C} groups. In some embodiments, Cy^{C} is thiazoleyl, wherein Cy^{C} is substituted with 0-5 -L^{C}-R^{C} groups.

In some embodiments, Cy^{C} is selected from the group consisting of:

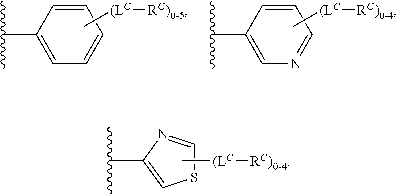

In some embodiments, a single instance of L^{C} is a covalent bond. In some embodiments, a single instance of L^{C} is an optionally substituted C₁₋₆ hydrocarbon chain, wherein 1 to 3 methylene units are optionally and independently replaced with —O— or —NR—. In some embodiments, L^{C} is optionally substituted with —(CH₂)₀₋₄R^{∘}, wherein R^{∘} is independently as defined above and described in classes and subclasses herein. In some embodiments, R^{∘} is hydrogen or C₁₋₆ aliphatic.

In some embodiments, a single instance of L^{C} is selected from the group consisting of: *—NH—, *—O—, *—CH₂—, *—CH₂C(CH₃)₂—, and *—CH₂CH₂—, wherein * represents the point of attachment to Cy^{C}. In some embodiments, a single instance of L^{C} is *—NH—, wherein * represents the point of attachment to Cy^{C}. In some embodiments, a single instance of L^{C} is *—O—, wherein * represents the point of attachment to Cy^{C}. In some embodiments, a single instance of L^{C} is *—CH₂—, wherein * represents the point of attachment to Cy^{C}. In some embodiments, a single instance of L^{C} is *—CH₂C(CH₃)₂—, wherein * represents the point of attachment to Cy^{C}.

In some embodiments, a single instance of R^{C} is selected from oxo, halogen, —CN, —C(O)₂R, —OR, or an optionally substituted group selected from C₁₋₆ aliphatic, a 3- to 7-membered saturated or partially unsaturated monocyclic carbocyclyl, a 5- or 6-membered heteroaryl having 1-2 heteroatoms selected from oxygen, nitrogen, or sulfur, a 3- to 7-membered saturated or partially unsaturated monocyclic heterocyclyl having 1-2 heteroatoms selected from oxygen, nitrogen, or sulfur, or a 6- to 12-membered saturated or unsaturated bicyclic heterocyclyl having 1-3 heteroatoms selected from oxygen, nitrogen, or sulfur.

In some embodiments, a single instance of R^{C} is oxo. In some embodiments, a single instance of R^{C} is halogen. In some embodiments, a single instance of R^{C} is fluorine. In some embodiments, a single instance of R^{C} is chlorine. In some embodiments, a single instance of R^{C} is —CN. In some embodiments, a single instance of R^{C} is —C(O)₂R. In some embodiments, a single instance of R^{C} is —COOH. In some embodiments, a single instance of R^{C} is —C(O)OCH₃. In some embodiments, a single instance of R^{C} is —C(O)OCH₂CH₃. In some embodiments, a single instance of R^{C} is —OR. In some embodiments, a single instance of R^{C} is —OH. In some embodiments, a single instance of R^{C} is —OMe. In some embodiments, a single instance of R^{C} is an optionally substituted C₁-6 aliphatic.

In some embodiments, a single instance of R^{C} is an optionally substituted 3- to 7-membered saturated or partially unsaturated monocyclic carbocyclyl. In some embodiments, a single instance of R^{C} is cyclopropyl. In some embodiments, Cy^{C} is substituted with 1-6 -L^{C}-R^{C} groups, wherein a single instance of R^{C} is cyclopropyl.

In some embodiments, a single instance of R^{C} is an optionally substituted 5- or 6-membered heteroaryl having 1-2 heteroatoms selected from oxygen, nitrogen, or sulfur. In some embodiments, a single instance of R^{C} is an optionally substituted 5-membered heteroaryl having 1-2 heteroatoms selected from oxygen, nitrogen, or sulfur. In some embodiments, a single instance of R^{C} is an optionally substituted 5-membered heteroaryl having 2 nitrogen heteroatoms. In some embodiments, a single instance of R^{C} is an optionally substituted pyrazolyl. In some embodiments, a single instance of R^{C} is pyrazolyl, optionally substituted with —(CH₂)₀₋₄R^{∘}; wherein R^{∘} is C₁₋₆ aliphatic (e.g., methyl or cyclopropyl). In some embodiments, a single instance of R^{C} is an optionally substituted imidazolyl. In some embodiments, a single instance of R^{C} is imidazolyl, optionally substituted with —(CH₂)₀₋₄R^{∘}; wherein R^{∘} is C₁₋₆ aliphatic (e.g., methyl or cyclopropyl).

In some embodiments, a single instance of R^{C} is an optionally substituted 6-membered heteroaryl having 1-2 heteroatoms selected from oxygen, nitrogen, or sulfur. In some embodiments, a single instance of R^{C} is an optionally substituted 6-membered heteroaryl having 1 nitrogen heteroatom. In some embodiments, a single instance of R^{C} is an optionally substituted pyridinonyl.

In some embodiments, a single instance of R^{C} is selected from the group consisting of:

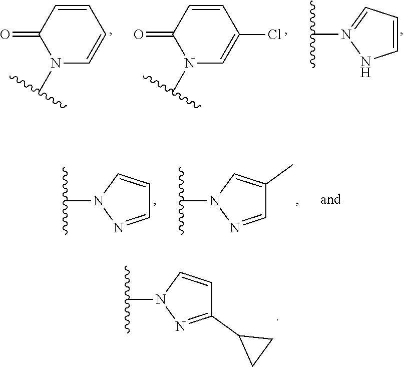

In some embodiments, a single instance of R^{C} is an optionally substituted 3- to 7-membered saturated or partially unsaturated monocyclic heterocyclyl having 1-2 heteroatoms selected from oxygen, nitrogen, or sulfur. In some embodiments, a single instance of R^{C} is an optionally substituted 5-membered saturated or partially unsaturated monocyclic heterocyclyl having 1-2 heteroatoms selected from oxygen, nitrogen, or sulfur. In some embodiments, a single instance of R^{C} is an optionally substituted 5-membered saturated or partially unsaturated monocyclic heterocyclyl having 2 nitrogen heteroatoms. In some embodiments, a single instance of R^{C} is an optionally substituted pyrrolidinyl. In some embodiments, a single instance of R^{C} is an optionally substituted imidazolidinyl. In some embodiments, a single instance of R^{C} is

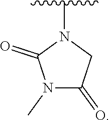 [SMILES: *N1CC(=O)N(C)C1=O]

In some embodiments, a single instance of R^{C} is

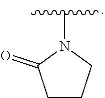 [SMILES: *N1CCCC1=O]

In some embodiments, a single instance of R^{C} is

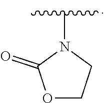 [SMILES: *N1CCOC1=O]

In some embodiments, Cy^{C} is substituted with 1-6 -L^{C}-R^{C} groups, wherein a single instance of R^{C} is

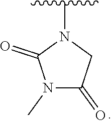 [SMILES: *N1CC(=O)N(C)C1=O]

In some embodiments, a single instance of R^{C} is an optionally substituted 6-membered saturated or partially unsaturated monocyclic heterocyclyl having 1-2 heteroatoms selected from oxygen, nitrogen, or sulfur. In some embodiments, a single instance of R^{C} is an optionally substituted 6-membered saturated or partially unsaturated monocyclic heterocyclyl having 2 nitrogen heteroatoms. In some embodiments, a single instance of R^{C} is an optionally substituted piperazinyl.

In some embodiments, a single instance of R^{C} is

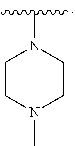 [SMILES: *N1CCN(C)CC1]

In some embodiments, a single instance of R^{C} is an optionally substituted 6- to 12-membered saturated or unsaturated bicyclic heterocyclyl having 1-3 heteroatoms selected from oxygen, nitrogen, or sulfur. In some embodiments, a single instance of R^{C} is an optionally substituted 6-membered saturated or unsaturated bicyclic heterocyclyl having 1-3 heteroatoms selected from oxygen, nitrogen, or sulfur. In some embodiments, a single instance of R^{C} is an optionally substituted 6-membered saturated or unsaturated bicyclic heterocyclyl having 1 nitrogen heteroatom. In some embodiments, a single instance of R^{C} is an optionally substituted azabicyclo[3.1.0]hexanyl. In some embodiments, a single instance of R^{C} is azabicyclo[3.1.0]hexanyl, optionally substituted with halogen. In some embodiments, a single instance of R^{C} is azabicyclo[3.1.0]hexanoneyl. In some embodiments, a single instance of R^{C} is

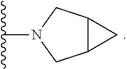 [SMILES: *N1CC2CC2C1]

In some embodiments, a single instance of R^{C} is

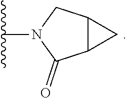 [SMILES: *N1CC2CC2C1=O]

In some embodiments, a single instance of R^{C} is

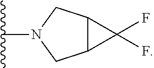 [SMILES: *N1CC2C(C1)C2(F)F]

In some embodiments, a provided compound is of Formula (II):

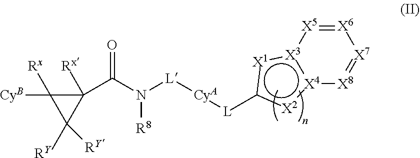

or a pharmaceutically acceptable salt thereof,
wherein
X¹ is N, CH, or C-L^{C}-R^{C};
each X² is independently selected from N, CH, or C-L^{C}-R^{C}.
X³ and X⁴ is independently N or C, wherein at least one of X³ or X⁴ is C;
each of X⁵, X⁶, X⁷, and X⁸ are independently selected from N, CH, or C-L^{C}-R^{C};
n is 1 or 2; and
each of Cy^{A}, Cy^{B}, L, L′, R^{x}, R^{x′}, R^{Y}, R^{Y′}, R⁸, L^{C} and R^{C} is defined and described in classes and subclasses herein, both singly and in combination.

It will be understood that, unless otherwise specified or prohibited by the foregoing definition of Formula (II), embodiments of variables Cy^{A}, Cy^{B}, L, L′, R^{x}, R^{x′}, R^{Y}, R^{Y′} R⁸, L^{C} and R^{C}as defined above and described in classes and subclasses herein, also apply to compounds of Formula (II), both singly and in combination.

In some embodiments, X¹ is N. In some embodiments, X¹ is CH. In some embodiments, X¹ is C-L^{C}-R^{C}, wherein L^{C} and R^{C} are as defined above and described in classes and subclasses herein, both singly and in combination.

In some embodiments, X³ is N or C and X⁴ is C. In some embodiments, X³ is C and X⁴ is N or C. In some embodiments, X³ is N and X⁴ is C. In some embodiments, X³ is C and X⁴ is C. In some embodiments, X³ is C and X⁴ is N.

In some embodiments, X⁵ is N. In some embodiments, X⁵ is CH. In some embodiments, X⁵ is C-L^{C}-R^{C}, wherein L^{C} and R^{C} are as defined above and described in classes and subclasses herein, both singly and in combination. In some embodiments, X⁶ is N. In some embodiments, X⁶ is CH. In some embodiments, X⁶ is C-L^{C}-R^{C}, wherein L^{C} and R^{C} are as defined above and described in classes and subclasses herein, both singly and in combination. In some embodiments, X⁷ is N. In some embodiments, X⁷ is CH. In some embodiments, X⁷ is C-L^{C}-R^{C}, wherein L^{C} and R^{C} are as defined above and described in classes and subclasses herein, both singly and in combination. In some embodiments, X⁸ is N. In some embodiments, X⁸ is CH. In some embodiments, X⁸ is C-L^{C}-R^{C}, wherein L^{C} and R^{C} are as defined above and described in classes and subclasses herein, both singly and in combination.

In some embodiments, n is 1. In some embodiments, n is 2.

In some embodiments, n is 1 and X⁸ is N. In some embodiments, n is 1 and X⁸ is CH. In some embodiments, n is 1 and X⁸ is C-L^{C}-R^{C}, wherein L^{C} and R^{C} are as defined above and described in classes and subclasses herein, both singly and in combination.

In some embodiments, n is 2 and each X² is independently selected from N, CH, or C-L^{C}-R^{C}, wherein L^{C} and R^{C} are as defined above and described in classes and subclasses herein, both singly and in combination. In some embodiments, n is 2 and one X² is N, and the other is CH. In some embodiments, n is 2 and both occurrences of X² are CH.

In some embodiments, a provided compound is of Formula (II-a) or Formula (II-b):

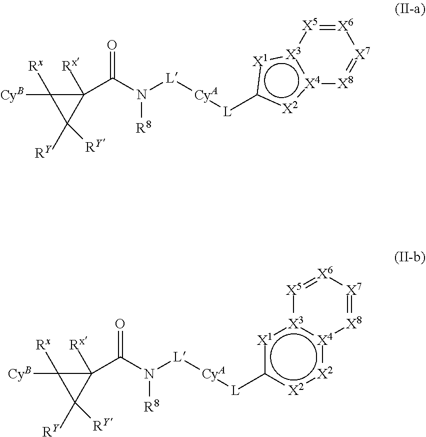

or a pharmaceutically acceptable salt thereof,
wherein each of Cy^{A}, Cy^{B}, L, L′, R^{x}, R^{x′}, R^{Y}, R^{Y′}, R⁸, X¹, X², X³, X⁴, X⁵, X⁶, X⁷, and X⁸ is defined and described in classes and subclasses herein, both singly and in combination.

It will be understood that, unless otherwise specified or prohibited by the foregoing definitions of Formulae (II-a) and (II-b) embodiments of variables Cy^{A}, Cy^{B}, L, L′, R^{x}, R^{x′}, R^{Y}, R^{Y′}, R⁸, X¹, X², X³, X⁴, X⁵, X⁶, X⁷, and X⁸ as defined above and described in classes and subclasses herein, also apply to compounds of Formulae (II-a) and (II-b) both singly and in combination.

In some embodiments, a provided compound is of Formula (II-a-1), Formula (II-a-2), Formula (II-a-3), or Formula (II-a-4).

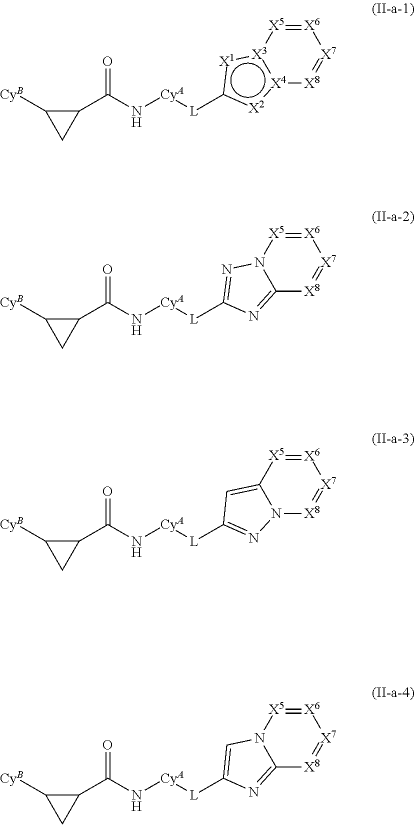

or a pharmaceutically acceptable salt thereof,
wherein each of Cy^{A}, Cy^{B}, L, X¹, X², X³, X⁴, X⁵, X⁶, X⁷, and X⁸ is defined and described in classes and subclasses herein, both singly and in combination.

It will be understood that, unless otherwise specified or prohibited by the foregoing definitions of Formulae (II-a-1), (II-a-2), (II-a-3), or (II-a-4), embodiments of variables Cy^{A}, Cy^{B}, L, X¹, X², X³, X⁴, X⁵, X⁶, X⁷, and X⁸ as defined above and described in classes and subclasses herein, also apply to compounds of Formulae (II-a-1), (II-a-2), (II-a-3), or (II-a-4), both singly and in combination.

In some embodiments, a provided compound is of Formula (II-b-1), Formula (II-b-2), Formula (II-b-3), or Formula (II-b-4).

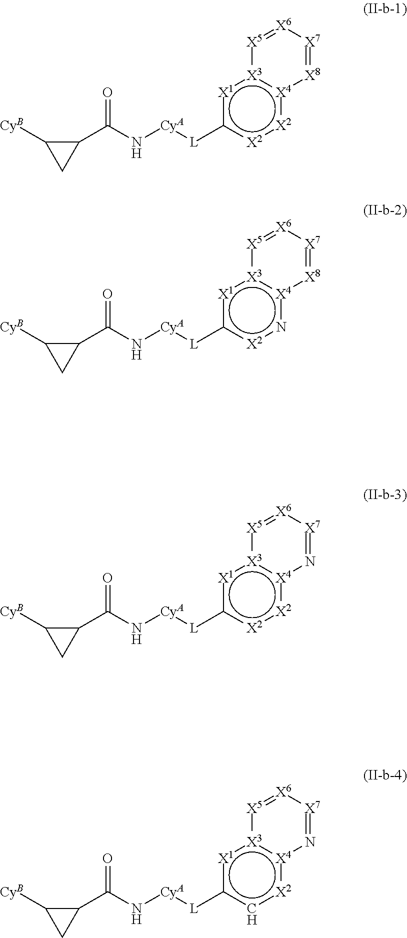

or a pharmaceutically acceptable salt thereof,
wherein each of Cy^{A}, Cy^{B}, L, X¹, X², X³, X⁴, X⁵, X⁶, X⁷, and X⁸ is defined and described in classes and subclasses herein, both singly and in combination.

It will be understood that, unless otherwise specified or prohibited by the foregoing definitions of Formulae (II-b-1), (II-b-2), (II-b-3), or (II-b-4) embodiments of variables Cy^{A}, Cy^{B}, L, X¹, X², X³, X⁴, X⁵, X⁶, X⁷, and X⁸ as defined above and described in classes and subclasses herein, also apply to compounds of Formulae (II-b-1), (II-b-2), or (II-b-3), or (II-b-4) both singly and in combination.

In some embodiments, a provided compound is of Formula (III), Formula (III-a), Formula (III-b), or Formula (III-c):

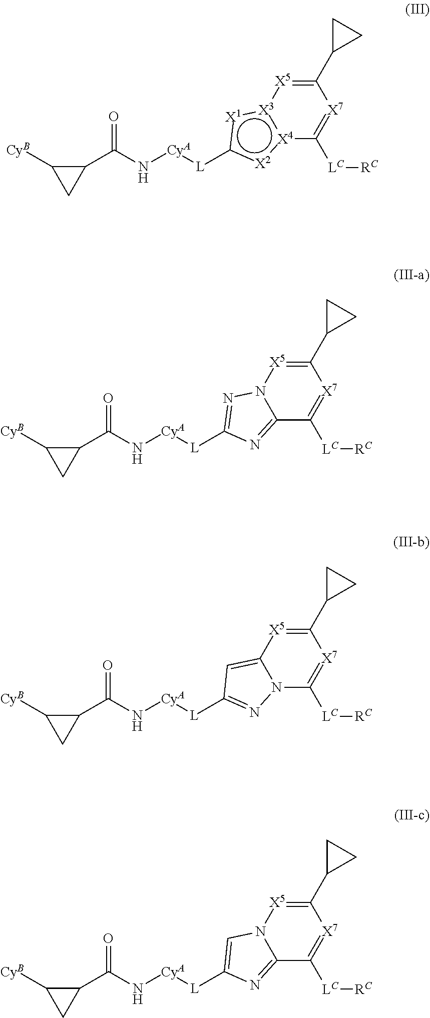

or a pharmaceutically acceptable salt thereof,
wherein each of Cy^{A}, Cy^{B}, L, L^{C}, R^{C}, X⁵, and X⁷ is defined and described in classes and subclasses herein, both singly and in combination.

It will be understood that, unless otherwise specified or prohibited by the foregoing definitions of Formulae (III), (III-a), (III-b), or (III-c), embodiments of variables Cy^{A}, Cy^{B}, L, L^{C}, R^{C}, X⁵, and X⁷ as defined above and described in classes and subclasses herein, also apply to compounds of Formulae Formula (III), (III-a), (III-b), or (III-c), both singly and in combination.

In some embodiments, a provided compound is of Formula (IV):

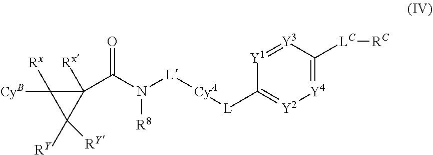

or a pharmaceutically acceptable salt thereof,
wherein
each of Y¹, Y², Y³, and Y⁴ is independently selected from N, CH, or C-L^{C}-R^{C}; and each of Cy^{A}, Cy^{B}, L, L′, R^{x}, R^{x′}, R^{Y}, R^{Y′}, R⁸, L^{C}, and R^{C} is defined and described in classes and subclasses herein, both singly and in combination.

It will be understood that, unless otherwise specified or prohibited by the foregoing definition of Formula (IV), embodiments of variables Cy^{A}, Cy^{B}, L, L′, R^{x}, R^{x′}, R^{Y}, R^{Y′}, R⁸, L^{C}, and R^{C} as defined above and described in classes and subclasses herein, also apply to compounds of Formula (IV), both singly and in combination.

In some embodiments, Y¹ is N. In some embodiments, Y¹ is CH. In some embodiments, Y¹ is C-L^{C}-R^{C}, wherein L^{C} and R^{C} are as defined above and described in classes and subclasses herein, both singly and in combination. In some embodiments, Y² is N. In some embodiments, Y² is CH. In some embodiments, Y² is C-L^{C}-R^{C}, wherein L^{C} and R^{C} are as defined above and described in classes and subclasses herein, both singly and in combination. In some embodiments, Y³ is N. In some embodiments, Y³ is CH. In some embodiments, Y³ is C-L^{C}-R^{C}, wherein L^{C} and R^{C} are as defined above and described in classes and subclasses herein, both singly and in combination. In some embodiments, Y⁴ is N. In some embodiments, Y⁴ is CH. In some embodiments, Y⁴ is C-L^{C}-R^{C}, wherein L^{C} and R^{C} are as defined above and described in classes and subclasses herein, both singly and in combination.

In some embodiments, a provided compound is of Formula (V-a) or Formula (V-b).

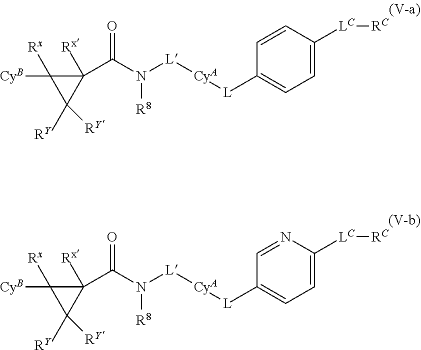

or a pharmaceutically acceptable salt thereof,
wherein each of Cy^{A}, Cy^{B}, L, L′, R^{x}, R^{x′}, R^{Y}, R^{Y′}, R⁸, L^{C}, and R^{C} is defined and described in classes and subclasses herein, both singly and in combination.

It will be understood that, unless otherwise specified or prohibited by the foregoing definitions of Formulae (V-a) and (V-b) embodiments of variables Cy^{A}, Cy^{B}, L, L′, R^{x}, R^{x′}, R^{Y}, R^{Y′}, R⁸, L^{C}, and R^{C} as defined above and described in classes and subclasses herein, also apply to compounds of Formulae (V-a) and (V-b), both singly and in combination.

In some embodiments, a provided compound is of Formula (VI), Formula (VI-a), or Formula (VI-b):

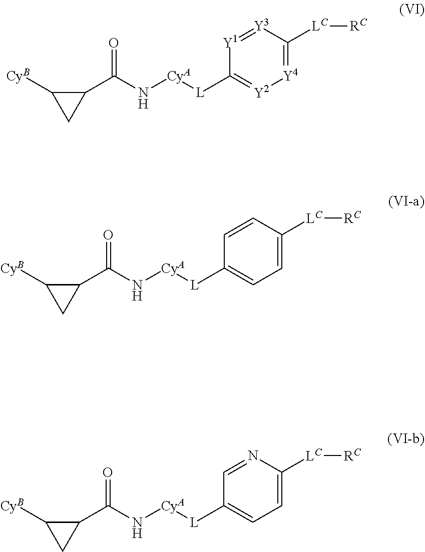

or a pharmaceutically acceptable salt thereof,
wherein each of Cy^{A}, Cy^{B}, L, L^{C}, and R^{C} is defined and described in classes and subclasses herein, both singly and in combination.

It will be understood that, unless otherwise specified or prohibited by the foregoing definitions of Formulae (VI), (VI-a), and (VI-b) embodiments of variables Cy^{A}, Cy^{B}, L, L^{C}, and R^{C} as defined above and described in classes and subclasses herein, also apply to compounds of Formulae (VI), (VI-a) and (VI-b), both singly and in combination.

In certain embodiments of provided compounds (i.e., of any species not otherwise defined and of any for Formula (I)-(VI-b), the moiety

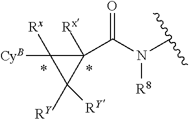

(including where one or more of R^{x}, R^{x′}, R^{Y}, R^{Y′} or R⁸ is hydrogen) is in the relative trans configuration with respect to the Cy^{B} and amide group attached to the two stereocenters marked with an *. In other words, it will be appreciated that “trans” in the context of the moiety:

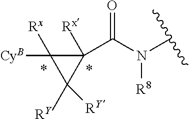

is meant a compound comprising a mixture of:

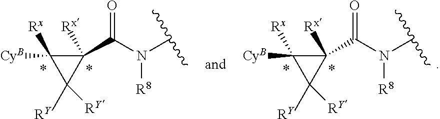

In some embodiments, such a mixture is a racemic mixture.

In certain embodiments of provided compounds (i.e., of any species not otherwise defined and of any of Formula (I)-(VI-b), the absolute stereochemistry of the moiety:

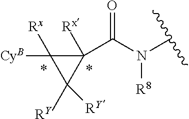

is as follows:

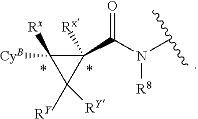

In certain embodiments of provided compounds (i.e., of any species not otherwise defined and of any of Formula (I)-(VI-b), the absolute stereochemistry of the moiety:

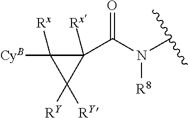

is as follows:

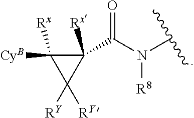

In some embodiments, a compound is selected from:
rac-(1S*,2S*)—N-(5-((4-((1H-pyrazol-1-yl)methyl)benzyl)oxy)pyridazin-3-yl)-2-(5-chloropyridin-3-yl)cyclopropane-1-carboxamide (I-1);
rac-(1S* ,2S*)-2-(3-chlorophenyl)-N-(5-((2-phenylthiazol-4-yl)methoxy)pyridazin-3-yl)cyclopropane-1-carboxamide (I-2);
rac-(1S*,2S*)—N-(5-(1-(4-((1H-pyrazol-1-yl)methyl)phenyl)ethoxy)pyridazin-3-yl)-2-(3 -chlorophenyl)cyclopropane-1-carboxamide (I-3);
rac-(1S*,2S*)-2-(3-chlorophenyl)-N-(5-((4-((3-cyclopropyl-1H-pyrazol-1-yl)methyl)benzyl)oxy)pyridazin-3-yl)cyclopropane-1-carboxamide (I-4);
rac-(1S*,2S*)—N-(5-((4-((1H-pyrazol-1-yl)methyl)benzyl)oxy)pyridazin-3-yl)-2-(2-chloropyridin-3-yl)cyclopropane-1-carboxamide (I-5);
rac-(11S*,2S*)—N-(5-((4-((1H-pyrazol-1-yl)methyl)benzyl)oxy)pyridazin-3-yl)-2-(H-pyrazol-4-yl)cyclopropane-1-carboxamide (I-6);
N-(5-((4-((1H-pyrazol-1-yl)methyl)benzyl)oxy)pyridazin-3-yl)-2′-oxospiro[cyclopropane-1,3′-indoline]-2-carboxamide (I-7);
rac-(1S*,2S*)—N-(5-((4-((1H-pyrazol-1-yl)methyl)benzyl)oxy)pyridazin-3-yl)-2-(2-aminopyridin-3-yl)cyclopropane-1-carboxamide (I-8);
rac-(1S*,2S*)—N-(1-(4-((1H-pyrazol-1-yl)methyl)benzyl)-1H-imidazol-4-yl)-2-(3-chlorophenyl)cyclopropane-1-carboxamide (I-9);
rac-(1S*,2S*)—N-(1-(4-((1H-pyrazol-1-yl)methyl)benzyl)-1H-pyrazol-4-yl)-2-(3-chlorophenyl)cyclopropane-1-carboxamide (I-10);
rac-(1S*,2S*)-2-(3-chlorophenyl)-N-(5-((2-(4-methyl-1H-pyrazol-1-yl)pyridin-4-yl)methoxy)pyridazin-3-yl)cyclopropanecarboxamide (I-11);
rac-(11S*,2S*)—N-(5-((4-(1H-imidazol-1-yl)benzyl)oxy)pyridazin-3-yl)-2-(3-chlorophenyl)cyclopropane-1-carboxamide (I-12);
rac-(1S*,2S*)—N-(5-((4-(1H-imidazol-2-yl)benzyl)oxy)pyridazin-3-yl)-2-(3-chlorophenyl)cyclopropane-1-carboxamide (I-13);
rac-(1S* ,2S*)-2-(3-chlorophenyl)-N-(5-((4-((2-oxopyridin-1 (2H)-yl)methyl)benzyl)amino)pyridazin-3-yl)cyclopropane-1-carboxamide (I-14);
(1S,2S)-2-(3-chlorophenyl)-N-(5-((4-((2-oxopyridin-1 (2H)-yl)methyl)benzyl)amino)pyridazin-3-yl)cyclopropane-1-carboxamide (I-15);
(1R,2R)-2-(3-chlorophenyl)-N-(5-((4-((2-oxopyridin-1 (2H)-yl)methyl)benzyl)amino)pyridazin-3-yl)cyclopropane-1-carboxamide (I-16);
rac-(1S*,2S*)—N-(5-((4-((5-chloro-2-oxopyridin-1 (2H)-yl)methyl)benzyl)amino)pyridazin-3-yl)-2-(3-chlorophenyl)cyclopropane-1-carboxamide (I-17);
rac-(11S*,2S*)—N-(5-((4-((1H-pyrazol-1-yl)methyl)benzyl)oxy)pyridazin-3-yl)-2-(3-chlorophenyl)cyclopropane-1-carboxamide (I-18);
rac-(1R*,2S*)-2-(3-chlorophenyl)-N-(5-((4-((2-oxopyridin-1 (2H)-yl)methyl)benzyl)amino)pyridazin-3-yl)cyclopropane-1-carboxamide (I-19);
rac-(1S*,2S*)—N-(5-(1-(4-((5-chloro-2-oxopyridin-1 (2H)-yl)methyl) phenyl)cyclopropyl)pyridazin-3-yl)-2-(3-chlorophenyl)cyclopropane-1-carboxamide (I-20);
rac-(1S*,2S*)-2-(3-chlorophenyl)-N-(6-((3-cyclopropylquinolin-6-yl)methyl)pyrimidin-4-yl)cyclopropane-1-carboxamide (I-21);
rac-(1S*,2S*)-2-(5-chloro-2-cyanophenyl)-N-(4-(((6-cyclopropylimidazo[1,2-a]pyrimidin-2-yl)methyl)amino)pyridin-2-yl)cyclopropane-1-carboxamide (I-22);
(1S,2S)-2-(3-chlorophenyl)-N-(4-(((5-cyclopropylthieno[2,3-b]pyridin-2-yl)methyl)amino)pyridin-2-yl)cyclopropane-1-carboxamide, formic acid salt (I-23);
(1S,2S)-2-(3-chlorophenyl)-N-(4-(((5-cyclopropylthieno[2,3-b]pyridin-2-yl)methyl)amino)pyridin-2-yl)cyclopropane-1-carboxamide;
rac-(1S*,2S*)-2-(5-chloro-2-cyanophenyl)-N-(6-(((6-cyclopropyl-[1,2,4]triazolo[1,5-a]pyridin-2-yl)methyl)amino)pyrimidin-4-yl)cyclopropane-1-carboxamide (I-24);
(1S,2S)-2-(3-chlorophenyl)-N-(4-(((6-cyclopropyl-[1,2,4]triazolo[1,5-a]pyridin-2-yl)methyl)amino)pyridin-2-yl)cyclopropane-1-carboxamide (I-25);
(1S,2S)-2-(3-chlorophenyl)-N-(6-(((6-cyclopropyl-[1,2,4]triazolo[1,5-a]pyridin-2-yl)methyl)amino)pyrimidin-4-yl)cyclopropane-1-carboxamide (I-26);
rac-(1S*,2S*)-2-(5-chloro-2-cyanophenyl)-N-(6-(((6-cyclopropyl-[1,2,4]triazolo[1,5-b]pyridazin-2-yl)methyl)amino)pyrimidin-4-yl)cyclopropane-1-carboxamide (I-27);
(1S,2S)-2-(5-chloro-2-cyanophenyl)-N-(6-(((6-cyclopropyl-[1,2,4]triazolo[1,5-b]pyridazin-2-yl)methyl)amino)pyrimidin-4-yl)cyclopropane-1-carboxamide (I-28);
(1S,2S)-2-(3-chlorophenyl)-N-(4-(((6-cyclopropyl-[1,2,4]triazolo[1,5-b]pyridazin-2-yl)methyl)amino)pyridin-2-yl)cyclopropane-1-carboxamide (I-29);
rac-(1S*,2S*)-2-(5-chloro-2-cyanophenyl)-N-(4-(((6-cyclopropyl-[1,2,4]triazolo[1,5-a]pyridin-2-yl)methyl)amino)pyridin-2-yl)cyclopropane-1-carboxamide (I-30);
(1S,2S)-2-(3-chlorophenyl)-N-(4-(((6-cyclopropyl-[1,2,4]triazolo[1,5-a]pyrimidin-2-yl)methyl)amino)pyridin-2-yl)cyclopropane-1-carboxamide (I-31);
(1S,2S)-2-(3-chlorophenyl)-N-(6-(((6-cyclopropyl-[1,2,4]triazolo[1,5-a]pyrimidin-2-yl)methyl)amino)pyrimidin-4-yl)cyclopropane-1-carboxamide (I-32);
(1S,2S)-2-(3-chlorophenyl)-N-(4-(((3-cyclopropylquinolin-6-yl)methyl)amino)pyridin-2-yl)cyclopropane-1-carboxamide (I-33);
(1S,2S)-2-(3-chlorophenyl)-N-(4-(((6-cyclopropylquinolin-3-yl)methyl)amino)pyridin-2-yl)cyclopropane-1-carboxamide (I-34);
(1S,2S)-2-(3-chlorophenyl)-N-(2-((6-cyclopropylimidazo[1,2-b]pyridazin-2-yl)methyl)-2H-pyrazolo[4,3-c]pyridin-4-yl)cyclopropane-1-carboxamide (I-35);
(1S,2S)-2-(3-chlorophenyl)-N-(4-(((6-cyclopropyl-5-oxo-5,6-dihydroimidazo[1,2-c]pyrimidin-2-yl)methyl)amino)pyridin-2-yl)cyclopropane-1-carboxamide (I-36);
(1S,2S)-2-(3-chlorophenyl)-N-(6-(((5-isopropoxyimidazo[1,2-c]pyrimidin-2-yl)methyl)amino)pyrimidin-4-yl)cyclopropane-1-carboxamide (I-37);
(1S,2S)-2-(3-chlorophenyl)-N-(6-(((6-isopropyl-5-oxo-5,6-dihydroimidazo[1,2-c]pyrimidin-2-yl)methyl)amino)pyrimidin-4-yl)cyclopropane-1-carboxamide (I-38);
(1S,2S)-2-(3-chlorophenyl)-N-(6-(((6-cyclopropyl-8-(4-methylpiperazin-1-yl)imidazo[1,2-b]pyridazin-2-yl)methyl)amino)pyrimidin-4-yl)cyclopropane-1-carboxamide (I-39);
(1S,2S)-2-(3-chlorophenyl)-N-(4-(((6-cyclopropyl-8-(4-methylpiperazin-1-yl)imidazo[1,2-b]pyridazin-2-yl)methyl)amino)pyridin-2-yl)cyclopropane-1-carboxamide (I-40);
rac-(1S*,2S*)-6′-chloro-N-(4-(((6-cyclopropyl-[1,2,4]triazolo[1,5-a]pyrimidin-2-yl)methyl)amino)pyridin-2-yl)-2′,3′-dihydrospiro[cyclopropane-1,1′-indene]-2-carboxamide (I-41);
rac-(1S*,2S*)-2-(5-chloro-2-cyanophenyl)-N-(4-(((6-cyclopropyl-[1,2,4]triazolo[1,5-a]pyrimidin-2-yl)methyl)amino)pyridin-2-yl)cyclopropane-1-carboxamide (I-42);
(1S,2S)-2-(3-chlorophenyl)-N-(6-(((6-cyclopropyl-8-(2-oxooxazolidin-3-yl)-[1,2,4]triazolo[1,5-a]pyridin-2-yl)methyl)amino)pyrimidin-4-yl)cyclopropane-1-carboxamide (I-43);
(1S,2S)-2-(3-chlorophenyl)-N-(6-(((6-cyclopropyl-8-(2-oxooxazolidin-3-yl)imidazo[1,2-b]pyridazin-2-yl)methyl)amino)pyrimidin-4-yl)cyclopropane-1-carboxamide (I-44);
(1S,2S)-2-(3-chlorophenyl)-N-(4-(((6-cyclopropyl-8-(4-methylpiperazin-1-yl)-[1,2,4]triazolo[1,5-a]pyridin-2-yl)methyl)amino)pyridin-2-yl)cyclopropane-1-carboxamide (I-45);
(1S,2S)-2-(3-chlorophenyl)-N-(4-(((6-cyclopropylimidazo[1,2-b]pyridazin-2-yl)methyl)amino)pyridin-2-yl)cyclopropane-1-carboxamide (I-46);
ethyl 3-(2-(((6-((1S,2S)-2-(3-chlorophenyl)cyclopropane-1-carboxamido)pyrimidin-4-yl)amino)methyl)-5-cyclopropylpyrazolo[1,5-a]pyridin-7-yl)propanoate (I-47);
3-(2-(((6-((1S,2S)-2-(3-chlorophenyl)cyclopropane-1-carboxamido)pyrimidin-4-yl)amino)methyl)-5-cyclopropylpyrazolo[1,5-a]pyridin-7-yl)propanoic acid (I-48);
(1S,2S)-2-(3-chlorophenyl)-N-(6-(((5-cyclopropyl-7-(2-oxooxazolidin-3-yl)pyrazolo[1,5-a]pyridin-2-yl)methyl)amino)pyrimidin-4-yl)cyclopropane-1-carboxamide (I-49);
(1S,2S)-2-(3-chlorophenyl)-N-(6-(((5-(cyclopropylamino)imidazo[1,2-c]pyrimidin-2-yl)methyl)amino)pyrimidin-4-yl)cyclopropane-1-carboxamide (I-50);
(1S,2S)-2-(3-chlorophenyl)-N-(6-(((5-cyclopropyl-7-(3-hydroxy-3-methylbutyl)pyrazolo[1,5-a]pyridin-2-yl)methyl)amino)pyrimidin-4-yl)cyclopropane-1-carboxamide (I-51);
(1S,2S)-2-(3-chlorophenyl)-N-(6-(((5-cyclopropyl-7-(2-oxopyrrolidin-1-yl)pyrazolo[1,5-a]pyridin-2-yl)methyl)amino)pyrimidin-4-yl)cyclopropane-1-carboxamide (I-52);
(1S,2S)-2-(3-chlorophenyl)-N-(6-(((5-cyclopropyl-7-(4-methylpiperazin-1-yl)pyrazolo[1,5-a]pyridin-2-yl)methyl)amino)pyrimidin-4-yl)cyclopropane-1-carboxamide (I-53);
(1S,2S)-2-(3-chlorophenyl)-N-(4-(((5-cyclopropyl-7-(4-methylpiperazin-1-yl)pyrazolo[1,5-a]pyridin-2-yl)methyl)amino)pyridin-2-yl)cyclopropane-1-carboxamide (I-54);
(1S,2S)-2-(3-chlorophenyl)-N-(6-(((7-(2-cyano-2-methylpropyl)-5-cyclopropylpyrazolo[1,5-a]pyridin-2-yl)methyl)amino)pyrimidin-4-yl)cyclopropane-1-carboxamide (I-55);
rac-(1S*,2S*)-2-(4-chloropyridin-2-yl)-N-(6-(((5-cyclopropyl-7-(2-oxooxazolidin-3-yl)pyrazolo[1,5-a]pyridin-2-yl)methyl)amino)pyrimidin-4-yl)cyclopropane-1-carboxamide (I-56);
rac-(1S*,2S*)-2-(4-chloropyridin-2-yl)-N-(6-(((5-cyclopropyl-7-(3-methyl-2,4-dioxoimidazolidin-1-yl)pyrazolo[1,5-a]pyridin-2-yl)methyl)amino)pyrimidin-4-yl)cyclopropane-1-carboxamide (I-57);
(1S,2S)-2-(3-chlorophenyl)-N-(6-(((6-cyclopropyl-8-(4-methylpiperazin-1-yl)-[1,2,4]triazolo[1,5-a]pyridin-2-yl)methyl)amino)pyrimidin-4-yl)cyclopropane-1-carboxamide (I-58);
(1S,2S)—N-(6-(((6-chloro-8-(4-methylpiperazin-1-yl)quinolin-3-yl)methyl)amino)pyrimidin-4-yl)-2-(3-chlorophenyl)cyclopropane-1-carboxamide (I-59);
(1S,2S)-2-(3-chlorophenyl)-N-(6-((2R,4S)-2-(6-cyclopropylimidazo[1,2-b]pyridazin-2-yl)-4-hydroxypyrrolidin-1-yl)pyrimidin-4-yl)cyclopropane-1-carboxamide (I-60);
(1S,2S)-2-(4-chloropyridin-2-yl)-N-(6-((2R,4S)-2-(6-cyclopropylimidazo[1,2-b]pyridazin-2-yl)-4-hydroxypyrrolidin-1-yl)pyrimidin-4-yl)cyclopropane-1-carboxamide (I-61);
(1S,2S)-2-(3-chlorophenyl)-N-(6-(((6-cyclopropyl-8-(3-methyl-2,4-dioxoimidazolidin-1-yl)imidazo[1,2-b]pyridazin-2-yl)methyl)amino)pyrimidin-4-yl)cyclopropane-1-carboxamide (I-62);
(1S,2S)-2-(4-chloropyridin-2-yl)-N-(6-(((6-cyclopropyl-8-(3-methyl-2,4-dioxoimidazolidin-1-yl)imidazo[1,2-b]pyridazin-2-yl)methyl)amino)pyrimidin-4-yl)cyclopropane-1-carboxamide (I-63);
(2R,4S)-1-(6-((1 S,2S)-2-(3-chlorophenyl)cyclopropane-1-carboxamido)pyrimidin-4-yl)-N-(4-cyclopropylphenyl)-4-hydroxypyrrolidine-2-carboxamide (I-64);
(2R,4S)-1-(6-((1 S,2S)-2-(3-chlorophenyl)cyclopropane-1-carboxamido)pyrimidin-4-yl)-N-(4-cyclopropylphenyl)-4-hydroxy-N-methylpyrrolidine-2-carboxamide (I-65);
(1S,2S)-2-(4-chloropyridin-2-yl)-N-(6-(((6-cyclopropylimidazo[1,2-a]pyrimidin-2-yl)methyl)amino)pyrimidin-4-yl)cyclopropane-1-carboxamide (I-66);
(1S,2S)—N-(6-((4S)-2-(6-(3-azabicyclo[3.1.0]hexan-3-yl)pyridin-3-yl)-4-hydroxypyrrolidin-1-yl)pyrimidin-4-yl)-2-(3-chlorophenyl)cyclopropane-1-carboxamide (I-67);
(1S,2S)-2-(4-chloropyridin-2-yl)-N-(6-(((6-cyclopropyl-8-(2,4-dioxoimidazolidin-1-yl)-[1,2,4]triazolo[1,5-a]pyridin-2-yl)methyl)amino)pyrimidin-4-yl)cyclopropane-1-carboxamide (I-68);
rac-(1S*,2S*)—N-(6-((4S)-2-(6-(3-azabicyclo[3.1.0]hexan-3-yl)pyridin-3-yl)-4-hydroxypyrrolidin-1-yl)pyrimidin-4-yl)-2-(4-methylpyrimidin-2-yl)cyclopropane-1-carboxamide (I-69);
rac-(1S*,2S*)—N-(6-((2S*,4S)-2-(6-(3-azabicyclo[3.1.0]hexan-3-yl)pyridin-3-yl)-4-hydroxypyrrolidin-1-yl)pyrimidin-4-yl)-2-(4-methylpyrimidin-2-yl)cyclopropane-1-carboxamide (I-70);
rac-(1S*,2S*)—N-(6-((2S,4S)-2-(6-(3-azabicyclo[3.1.0]hexan-3-yl)pyridin-3-yl)-4-hydroxypyrrolidin-1-yl)pyrimidin-4-yl)-2-(4-methylpyrimidin-2-yl)cyclopropane-1-carboxamide (I-70a);
rac-(1S*,2S*)—N-(6-((2R^{•},4S)-2-(6-(3-azabicyclo[3.1.0]hexan-3-yl)pyridin-3-yl)-4-hydroxypyrrolidin-1-yl)pyrimidin-4-yl)-2-(4-methylpyrimidin-2-yl)cyclopropane-1-carboxamide (I-71);
rac-(1S*,2S*)—N-(6-((2R,4S)-2-(6-(3-azabicyclo[3.1.0]hexan-3-yl)pyridin-3-yl)-4-hydroxypyrrolidin-1-yl)pyrimidin-4-yl)-2-(4-methylpyrimidin-2-yl)cyclopropane-1-carboxamide (I-71a);
rac-(1S*,2S*)—N-(6-((2R,4S)-2-(6-cyclopropylimidazo[1,2-a]pyrimidin-2-yl)-4-hydroxypyrrolidin-1-yl)pyrimidin-4-yl)-2-(4-methylpyrimidin-2-yl)cyclopropane-1-carboxamide (I-72);
(1S,2S)-2-(4-chloropyridin-2-yl)-N-(6-((2R,4S)-2-(6-cyclopropylimidazo[1,2-a]pyrimidin-2-yl)-4-hydroxypyrrolidin-1-yl)pyrimidin-4-yl)cyclopropane-1-carboxamide (I-73);
(1S* ,2S*)—N-(6-((2R,4S)-2-(6-cyclopropylimidazo[1,2-a]pyrimidin-2-yl)-4-hydroxypyrrolidin-1-yl)pyrimidin-4-yl)-2-(4-methylpyridin-2-yl)cyclopropane-1-carboxamide, isomer 1 (I-74);
(1S,2S)—N-(6-((2R,4S)-2-(6-cyclopropylimidazo[1,2-a]pyrimidin-2-yl)-4-hydroxypyrrolidin-1-yl)pyrimidin-4-yl)-2-(4-methylpyridin-2-yl)cyclopropane-1-carboxamide (I-74a);
(1S*,2S*)—N-(6-((2R,4S)-2-(6-cyclopropylimidazo[1,2-a]pyrimidin-2-yl)-4-hydroxypyrrolidin-1-yl)pyrimidin-4-yl)-2-(4-methylpyridin-2-yl)cyclopropane-1-carboxamide, isomer 2 (I-75);
(1R,2R)—N-(6-((2R,4S)-2-(6-cyclopropylimidazo[1,2-a]pyrimidin-2-yl)-4-hydroxypyrrolidin-1-yl)pyrimidin-4-yl)-2-(4-methylpyridin-2-yl)cyclopropane-1-carboxamide (I-75a);
rac-(1S*,2S*)—N-(6-((2R,4S)-2-(6-cyclopropylimidazo[1,2-a]pyrimidin-2-yl)-4-hydroxypyrrolidin-1-yl)pyrimidin-4-yl)-2-(3-fluoro-4-methylpyridin-2-yl)cyclopropane-1-carboxamide (I-76);
rac-(1S*,2S*)—N-(4-(((6-cyclopropylimidazo[1,2-a]pyrimidin-2-yl)methyl)amino)pyrimidin-2-yl)-2-(4-methylpyrimidin-2-yl)cyclopropane-1-carboxamide (I-77);
rac-(1S*,2S*)—N-(6-(((6-cyclopropyl-[1,2,4]triazolo[1,5-a]pyrimidin-2-yl)methyl)amino)pyrimidin-4-yl)-2-(4-methylpyrimidin-2-yl)cyclopropane-1-carboxamide (I-78);
rac-(1S*,2S*)—N-(6-(((6-(3-azabicyclo[3.1.0]hexan-3-yl)pyridin-3-yl)methyl)amino)pyrimidin-4-yl)-2-(4-methylpyrimidin-2-yl)cyclopropane-1-carboxamide (I-79);
rac-(1S*,2S*)—N-(6-(((6-cyclopropyl-8-(3-methyl-2,4-dioxoimidazolidin-1-yl)imidazo[1,2-a]pyrazin-2-yl)methyl)amino)pyrimidin-4-yl)-2-(4-methylpyrimidin-2-yl)cyclopropane-1-carboxamide (I-80);
(1S*,2S*)—N-(6-(((6-cyclopropyl-8-(3-methyl-2,4-dioxoimidazolidin-1-yl)imidazo[1,2-a]pyrazin-2-yl)methyl)amino)pyrimidin-4-yl)-2-(4-methylpyrimidin-2-yl)cyclopropane-1-carboxamide, first eluting isomer (I-81);
(1S,2S)—N-(6-(((6-cyclopropyl-8-(3-methyl-2,4-dioxoimidazolidin-1-yl)imidazo[1,2-a]pyrazin-2-yl)methyl)amino)pyrimidin-4-yl)-2-(4-methylpyrimidin-2-yl)cyclopropane-1-carboxamide (I-81a);
(1S* ,2S*)—N-(6-(((6-cyclopropyl-8-(3-methyl-2,4-dioxoimidazolidin-1-yl)imidazo[1,2-a]pyrazin-2-yl)methyl)amino)pyrimidin-4-yl)-2-(4-methylpyrimidin-2-yl)cyclopropane-1-carboxamide, second eluting isomer (I-82);
(1R,2R)—N-(6-(((6-cyclopropyl-8-(3-methyl-2,4-dioxoimidazolidin-1-yl)imidazo[1,2-a]pyrazin-2-yl)methyl)amino)pyrimidin-4-yl)-2-(4-methylpyrimidin-2-yl)cyclopropane-1-carboxamide (I-82a);
(1S,2S)—N-(6-(((6-cyclopropyl-8-(3-methyl-2,4-dioxoimidazolidin-1-yl)imidazo[1,2-a]pyrazin-2-yl)methyl)(methyl)amino)pyrimidin-4-yl)-2-(4-methylpyrimidin-2-yl)cyclopropane-1-carboxamide (I-83);
rac-(1S*,2S*)—N-(4-(((6-cyclopropyl-8-(3-methyl-2,4-dioxoimidazolidin-1-yl)imidazo[1,2-a]pyrazin-2-yl)methyl)amino)-5-fluoropyrimidin-2-yl)-2-(4-methylpyrimidin-2-yl)cyclopropane-1-carboxamide (I-84);
(1S,2S)—N-(6-(((6-cyclopropyl-8-(2-oxopyrrolidin-1-yl)imidazo[1,2-a]pyrazin-2-yl)methyl)(methyl)amino)pyrimidin-4-yl)-2-(4-methylpyrimidin-2-yl)cyclopropane-1-carboxamide (I-85);
(1S,2S)—N-(6-(((5-cyclopropyl-7-(3-methyl-2,4-dioxoimidazolidin-1-yl)-1H-benzo[d]imidazol-2-yl)methyl)(methyl)amino)-2-methylpyrimidin-4-yl)-2-(4-methylpyrimidin-2-yl)cyclopropane-1-carboxamide (I-86);
(1S,2S)—N-(4-((6-cyclopropyl-8-(3-methyl-2,4-dioxoimidazolidin-1-yl)imidazo[1,2-a]pyrazin-2-yl)
methoxy)-6-methyl-1,3,5-triazin-2-yl)-2-(4-methylpyrimidin-2-yl)cyclopropane-1-carboxamide (I-87);
(1S,2S)—N-(6-(((6-(6,6-difluoro-3-azabicyclo[3.1.0]hexan-3-yl)-2-methylpyridin-3-yl)methyl)amino)-2-methylpyrimidin-4-yl)-2-(4-methylpyrimidin-2-yl)cyclopropane-1-carboxamide (I-88);
(1S,2S)—N-(6-((1-(6-cyclopropyl-8-(3-methyl-2,4-dioxoimidazolidin-1-yl)imidazo[1,2-b]pyridazin-2-yl)ethyl)amino)-2-methylpyrimidin-4-yl)-2-(4-methylpyrimidin-2-yl)cyclopropane-1-carboxamide (I-89);
(1S,2S)—N-(6-(((6-cyclopropylimidazo[1,2-a]pyrimidin-2-yl)methyl)(methyl)amino)-2-methylpyrimidin-4-yl)-2-(4-methylpyrimidin-2-yl)cyclopropane-1-carboxamide (I-90);
(1S,2S)—N-(6-(((3-chloro-8-fluoroquinolin-6-yl)methyl)amino)-2-methylpyrimidin-4-yl)-2-(4-methylpyrimidin-2-yl)cyclopropane-1-carboxamide (I-91);
(1S,2S)—N-(5-((3-chloro-8-fluoroquinolin-6-yl)methoxy)pyridazin-3-yl)-2-(4-methylpyrimidin-2-yl)cyclopropane-1-carboxamide (I-92);
(1S,2S)—N-(6-(((3-cyclopropylquinolin-6-yl)methyl)amino)pyrimidin-4-yl)-2-(4-methylpyrimidin-2-yl)cyclopropane-1-carboxamide (I-93);
(1S,2S)—N-(5-((6-cyclopropylimidazo[1,2-a]pyrimidin-2-yl)methoxy)pyridazin-3-yl)-2-(4-methylpyrimidin-2-yl)cyclopropane-1-carboxamide (I-94);
(1S,2S)—N-(6-(((R^{•})-1-(5-cyclopropyl-7-(3-methyl-2,4-dioxoimidazolidin-1-yl)pyrazolo[1,5-a]pyridin-2-yl)ethyl)amino)pyrimidin-4-yl)-2-(4-methylpyrimidin-2-yl)cyclopropane-1-carboxamide,
isomer 1 (I-95);
(1S,2S)—N-(6-(((R)-1-(5-cyclopropyl-7-(3-methyl-2,4-dioxoimidazolidin-1-yl)pyrazolo[1,5-a]pyridin-2-yl)ethyl)amino)pyrimidin-4-yl)-2-(4-methylpyrimidin-2-yl)cyclopropane-1-carboxamide
(1-95a);
(1S,2S)—N-(6-(((R^{•})-1-(5-cyclopropyl-7-(3-methyl-2,4-dioxoimidazolidin-1-yl)pyrazolo[1,5-a]pyridin-2-yl)ethyl)amino)pyrimidin-4-yl)-2-(4-methylpyrimidin-2-yl)cyclopropane-1-carboxamide,
isomer 2 (I-96);
(1S,2S)—N-(6-(((S)-1-(5-cyclopropyl-7-(3-methyl-2,4-dioxoimidazolidin-1-yl)pyrazolo[1,5-a]pyridin-2-yl)ethyl)amino)pyrimidin-4-yl)-2-(4-methylpyrimidin-2-yl)cyclopropane-1-carboxamide (I-96a);
(1S,2S)—N-(5-((6-cyclopropyl-8-(3-methyl-2,4-dioxoimidazolidin-1-yl)imidazo[1,2-b]pyridazin-2-yl)methoxy)pyridazin-3-yl)-2-(4-methylpyrimidin-2-yl)cyclopropane-1-carboxamidee (I-97);
(1S,2S)—N-(6-(((3-cyclopropyl-8-(3-methyl-2,4-dioxoimidazolidin-1-yl)quinolin-6-yl)methyl)amino)pyrimidin-4-yl)-2-(4-methylpyrimidin-2-yl)cyclopropane-1-carboxamide (I-98);
(1S,2S)—N-(6-(((5-cyclopropyl-7-(3-methyl-2,4-dioxoimidazolidin-1-yl)pyrazolo[1,5-a]pyridin-2-yl)methyl)amino)-2-methylpyrimidin-4-yl)-2-(4-methylpyrimidin-2-yl)cyclopropane-1-carboxamide (I-99);
(1S,2S)—N-(5-((5-cyclopropyl-7-(3-methyl-2,4-dioxoimidazolidin-1-yl)pyrazolo[1,5-a]pyridin-2-yl)methoxy)pyridazin-3-yl)-2-(4-methylpyrimidin-2-yl)cyclopropane-1-carboxamide (I-100);
(1S,2S)—N-(6-(((5-cyclopropyl-7-(3-methyl-2,4-dioxoimidazolidin-1-yl)pyrazolo[1,5-a]pyridin-2-yl)methyl)amino)pyrimidin-4-yl)-2-(4-methylpyrimidin-2-yl)cyclopropane-1-carboxamide (I-101);
(1S,2S)—N-(5-(((R^{•})-1-(5-cyclopropyl-7-(3-methyl-2,4-dioxoimidazolidin-1-yl)pyrazolo[1,5-a]pyridin-2-yl)ethyl)amino)-1,2,4-triazin-3-yl)-2-(4-methylpyrimidin-2-yl)cyclopropane-1-carboxamide (I-102);
(1S,2S)—N-(5-(((R)-1-(5-cyclopropyl-7-(3-methyl-2,4-dioxoimidazolidin-1-yl)pyrazolo[1,5-a]pyridin-2-yl)ethyl)amino)-1,2,4-triazin-3-yl)-2-(4-methylpyrimidin-2-yl)cyclopropane-1-carboxamide (I-102a);
(1S,2S)—N-(5-(((S)-1-(5-cyclopropyl-7-(3-methyl-2,4-dioxoimidazolidin-1-yl)pyrazolo[1,5-a]pyridin-2-yl)ethyl)amino)-1,2,4-triazin-3-yl)-2-(4-methylpyrimidin-2-yl)cyclopropane-1-carboxamide (I-102b);
(1S,2S)—N-(6-(((1R^{•})-1-(5-cyclopropyl-7-(2-oxo-3-azabicyclo[3.1.0]hexan-3-yl)pyrazolo[1,5-a]pyridin-2-yl)ethyl)amino)pyrimidin-4-yl)-2-(4-methylpyrimidin-2-yl)cyclopropane-1-carboxamide (I-103);
(1S,2S)—N-(6-(((1R)-1-(5-cyclopropyl-7-(2-oxo-3-azabicyclo[3.1.0]hexan-3-yl)pyrazolo[1,5-a]pyridin-2-yl)ethyl)amino)pyrimidin-4-yl)-2-(4-methylpyrimidin-2-yl)cyclopropane-1-carboxamide
(1-103a);
(1S,2S)—N-(6-(((1S)-1-(5-cyclopropyl-7-(2-oxo-3-azabicyclo[3.1.0]hexan-3-yl)pyrazolo[1,5-a]pyridin-2-yl)ethyl)amino)pyrimidin-4-yl)-2-(4-methylpyrimidin-2-yl)cyclopropane-1-carboxamide (I-103b);
(1S,2S)—N-(6-(((R^{•})-1-(5-cyclopropyl-7-(3-methyl-2,4-dioxoimidazolidin-1-yl)pyrazolo[1,5-a]pyrimidin-2-yl)ethyl)amino)pyrimidin-4-yl)-2-(4-methylpyrimidin-2-yl)cyclopropane-1-carboxamide (I-104);
(1S,2S)—N-(6-(((R)-1-(5-cyclopropyl-7-(3-methyl-2,4-dioxoimidazolidin-1-yl)pyrazolo[1,5-a]pyrimidin-2-yl)ethyl)amino)pyrimidin-4-yl)-2-(4-methylpyrimidin-2-yl)cyclopropane-1-carboxamide (I-104a);
(1S,2S)—N-(6-(((S)-1-(5-cyclopropyl-7-(3-methyl-2,4-dioxoimidazolidin-1-yl)pyrazolo[1,5-a]pyrimidin-2-yl)ethyl)amino)pyrimidin-4-yl)-2-(4-methylpyrimidin-2-yl)cyclopropane-1-carboxamide (I-104b);
(1S,2S)—N-(5-(((1R^{•})-1-(5-cyclopropyl-7-(2-oxo-3-azabicyclo[3.1.0]hexan-3-yl)pyrazolo[1,5-a]pyridin-2-yl)ethyl)amino)-1,2,4-triazin-3-yl)-2-(4-methylpyrimidin-2-yl)cyclopropane-1-carboxamide (I-105);
(1S,2S)—N-(5-(((1R)-1-(5-cyclopropyl-7-(2-oxo-3-azabicyclo[3.1.0]hexan-3-yl)pyrazolo[1,5-a]pyridin-2-yl)ethyl)amino)-1,2,4-triazin-3-yl)-2-(4-methylpyrimidin-2-yl)cyclopropane-1-carboxamide (I-105a);
(1S,2S)—N-(5-(((1S)-1-(5-cyclopropyl-7-(2-oxo-3-azabicyclo[3.1.0]hexan-3-yl)pyrazolo[1,5-a]pyridin-2-yl)ethyl)amino)-1,2,4-triazin-3-yl)-2-(4-methylpyrimidin-2-yl)cyclopropane-1-carboxamide (I-105b);
(1S,2S)—N-(6-(((R^{•})-1-(5-methyl-7-(3-methyl-2,4-dioxoimidazolidin-1-yl)pyrazolo[1,5-a]pyridin-2-yl)ethyl)amino)pyrimidin-4-yl)-2-(4-methylpyrimidin-2-yl)cyclopropane-1-carboxamide (I-106);
(1S,2S)—N-(6-(((R)-1-(5-methyl-7-(3-methyl-2,4-dioxoimidazolidin-1-yl)pyrazolo[1,5-a]pyridin-2-yl)ethyl)amino)pyrimidin-4-yl)-2-(4-methylpyrimidin-2-yl)cyclopropane-1-carboxamide (I-106a);
(1S,2S)—N-(6-(((S)-1-(5-methyl-7-(3-methyl-2,4-dioxoimidazolidin-1-yl)pyrazolo[1,5-a]pyridin-2-yl)ethyl)amino)pyrimidin-4-yl)-2-(4-methylpyrimidin-2-yl)cyclopropane-1-carboxamide (I-106b);
(1S,2S)—N-(6-(((R^{•})-1-(6-cyclopropyl-8-(3-methyl-2,4-dioxoimidazolidin-1-yl)-[1,2,4]triazolo[1,5-a]pyridin-2-yl)ethyl)amino)pyrimidin-4-yl)-2-(4-methylpyrimidin-2-yl)cyclopropane-1-carboxamide (I-107);
(1S,2S)—N-(6-(((R)-1-(6-cyclopropyl-8-(3-methyl-2,4-dioxoimidazolidin-1-yl)-[1,2,4]triazolo[1,5-a]pyridin-2-yl)ethyl)amino)pyrimidin-4-yl)-2-(4-methylpyrimidin-2-yl)cyclopropane-1-carboxamide (I-107a);
(1S,2S)—N-(6-(((R^{•})-1-(6-cyclopropyl-8-(3-methyl-2,4-dioxoimidazolidin-1-yl)-[1,2,4]triazolo[1,5-a]pyridin-2-yl)ethyl)amino)pyrimidin-4-yl)-2-(4-methylpyrimidin-2-yl)cyclopropane-1-carboxamide (I-108); or
(1S,2S)—N-(6-(((S)-1-(6-cyclopropyl-8-(3-methyl-2,4-dioxoimidazolidin-1-yl)-[1,2,4]triazolo[1,5-a]pyridin-2-yl)ethyl)amino)pyrimidin-4-yl)-2-(4-methylpyrimidin-2-yl)cyclopropane-1-carboxamide (I-108a);
   or a pharmaceutically acceptable salt thereof.

Compounds explicitly disclosed herein may be claimed as an individual compound, including where there is no reference to stereochemistry.

Processes for preparing compounds of the disclosure are described herein below.

### C. Pharmaceutical Compositions

In another aspect, the present invention provides pharmaceutical compositions comprising a compound of the present disclosure including Formulae (I)-(VI-b) or a compound of Formulae (I)-(VI-b) or a compound disclosed in the examples in combination with a pharmaceutically acceptable excipient (e.g., carrier).

The pharmaceutical compositions include optical isomers, diastereomers, or pharmaceutically acceptable salts of the inhibitors disclosed herein. A compound of Formulae (I)-(VI-b) included in the pharmaceutical composition may be covalently attached to a carrier moiety, as described above. Alternatively, a compound of Formulae (I)-(VI-b) included in the pharmaceutical composition is not covalently linked to a carrier moiety.

A “pharmaceutically acceptable carrier,” as used herein refers to pharmaceutical excipients, for example, pharmaceutically, physiologically, acceptable organic or inorganic carrier substances suitable for enteral or parenteral application that do not deleteriously react with the active agent. Suitable pharmaceutically acceptable carriers include water, salt solutions (such as Ringer's solution), alcohols, oils, gelatins, and carbohydrates such as lactose, amylose or starch, fatty acid esters, hydroxymethycellulose, and polyvinyl pyrrolidine. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention.

The compounds of the invention can be administered alone or can be coadministered to the subject. Coadministration is meant to include simultaneous or sequential administration of the compounds individually or in combination (more than one compound). The preparations can also be combined, when desired, with other active substances (e.g. to reduce metabolic degradation).

In some embodiments, a compound as described herein can be incorporated into a pharmaceutical composition for administration by methods known to those skilled in the art and described herein for provided compounds.

### D. Formulations

Compounds of the present invention can be prepared and administered in a wide variety of oral, parenteral, and topical dosage forms. Thus, the compounds of the present invention can be administered by injection (e.g. intravenously, intramuscularly, intracutaneously, subcutaneously, intraduodenally, or intraperitoneally). In some embodiments compounds of the present disclosure are administered orally. Also, the compounds described herein can be administered by inhalation, for example, intranasally. Additionally, the compounds of the present invention can be administered transdermally. It is also envisioned that multiple routes of administration (e.g., intramuscular, oral, transdermal) can be used to administer the compounds of the invention. Accordingly, the present invention also provides pharmaceutical compositions comprising a pharmaceutically acceptable carrier or excipient and one or more compounds of the invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substance that may also act as diluents, flavoring agents, binders, preservatives, tablet disintegrating agents, or an encapsulating material.

In powders, the carrier is a finely divided solid in a mixture with the finely divided active component. In tablets, the active component is mixed with the carrier having the necessary binding properties in suitable proportions and compacted in the shape and size desired.

The powders and tablets preferably contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the compounds of the invention are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. The compounds of the invention can also be incorporated into liposomes or administered via transdermal pumps or patches. Pharmaceutical admixtures suitable for use in the present invention include those described, for example, in Pharmaceutical Sciences (17th Ed., Mack Pub. Co., Easton, PA) and WO 96/05309, the teachings of both of which are hereby incorporated by reference.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted according to the particular application and the potency of the active component. The composition can, if desired, also contain other compatible therapeutic agents.

Some compounds may have limited solubility in water and therefore may require a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight.

Viscosity greater than that of simple aqueous solutions may be desirable to decrease variability in dispensing the formulations, to decrease physical separation of components of a suspension or emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

The compositions of the present invention may additionally include components to provide sustained release and/or comfort. Such components include high molecular weight, anionic mucomimetic polymers, gelling polysaccharides, and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

### E. Effective Dosages

Pharmaceutical compositions provided by the present invention include compositions wherein the active ingredient is contained in a therapeutically effective amount, i.e., in an amount effective to achieve its intended purpose. The actual amount effective for a particular application will depend, inter alia, on the condition being treated. For example, when administered in methods to treat HAE, such compositions will contain an amount of active ingredient effective to achieve the desired result (e.g. inhibiting PKa and/or decreasing the amount of bradykinin in a subject).

The dosage and frequency (single or multiple doses) of compound administered can vary depending upon a variety of factors, including route of administration; size, age, sex, health, body weight, body mass index, and diet of the recipient; nature and extent of symptoms of the disease being treated (e.g., the disease responsive to PKa inhibition); presence of other diseases or other health-related problems; kind of concurrent treatment; and complications from any disease or treatment regimen. Other therapeutic regimens or agents can be used in conjunction with the methods and compounds of the invention.

For any provided compound or test agent, the therapeutically effective amount can be initially determined from cell culture assays. Target concentrations will be those concentrations of active compound(s) that are capable of decreasing PKa enzymatic activity as measured, for example, using the methods described.

Therapeutically effective amounts for use in humans may be determined from animal models. For example, a dose for humans can be formulated to achieve a concentration that has been found to be effective in animals. The dosage in humans can be adjusted by monitoring PKa inhibition and adjusting the dosage upwards or downwards, as described above.

Dosages may be varied depending upon the requirements of the patient and the compound being employed. The dose administered to a patient, in the context of the present invention, should be sufficient to effect a beneficial therapeutic response in the patient over time. The size of the dose also will be determined by the existence, nature, and extent of any adverse side effects.

In one aspect, compounds provided herein display one or more improved pharmacokinetic (PK) properties (e.g., Cₘₐₓ, tₘₐₓ, Cₘᵢₙ, t_{1/2}, AUC, CL, bioavailability, etc.) when compared to a reference compound. In some embodiments, a reference compound is a PKa inhibitor known in the art. In some embodiments, a reference compound is a PKa inhibitor selected from those disclosed in PCT Publication Number WO 2019/178129.

In some embodiments a compound of the disclosure or a pharmaceutical composition comprising the same is provided as a unit dose.

### F. Methods of Treatment

The present disclosure provides compounds and pharmaceutical compositions comprising the same for use in medicine i.e. for use in treatment. The present disclosure further provides the use of any compounds described herein for inhibiting the activity of PKa, which would be beneficial to treatment of PKa-mediated diseases and conditions. Exemplary PKa-mediated disorders include edema, which refers to swelling in the whole body of a subject or a part thereof due to inflammation or injury when small blood vessels become leaky and releases fluid into nearby tissues. In some examples, the edema is HAE. In other examples, the edema occurs in eyes, e.g., diabetic macular edema (DME). The present disclosure provides methods of inhibiting the activity of PKa. In certain embodiments, the application provides a method of inhibiting the activity of PKa in vitro via contacting any of the compounds described herein with PKa molecules in a sample, such as a biological sample. In certain embodiments, the application provides a method of inhibiting the activity of PKa in vivo via delivering an effective amount of any of the compounds described herein to a subject in need of the treatment through a suitable route.

In certain embodiments, the methods comprise administering to a subject in need thereof (e.g., a subject such as a human patient, for example with edema) any of the compounds described herein or a pharmaceutically acceptable salt thereof. In certain embodiments, the methods comprise administering a compound of Formulae (I)-(VI-b), or a pharmaceutically acceptable salt or composition thereof, to a subject in need thereof. In some embodiments, the method comprises administering a pharmaceutical composition comprising a compound of Formulae (I)-(VI-b), or a pharmaceutically acceptable salt to a subject in need thereof.

In certain embodiments, the subject to be treated by any of the methods described herein is a human patient having, suspected of having, or at risk for edema, for example, HAE or diabetic macular edema (DME). A subject having an edema can be identified by routine medical examination, e.g., laboratory tests. A subject suspected of having an edema might show one or more symptoms of the disease/disorder. A subject at risk for edema can be a subject having one or more of the risk factors associated with the disease, for example, deficiency in C1-INH as for HAE.

In certain embodiments, provided herein are methods of alleviating one or more symptoms of HAE in a human patient who is suffering from an HAE attack. Such a patient can be identified by routine medical procedures. An effective amount of one or more of the provided compounds can be given to the human patient via a suitable route, for example, those described herein. The compounds described herein may be used alone, or may be used in combination with other anti-HAE agents, for example, a C1 esterase inhibitor (e.g., Cinryze® or Berinert®), a PKa inhibitor (e.g., ecallantide or lanadelumab) or a bradykinin B2 receptor antagonist (e.g., Firazyr®).

In some embodiments, provided herein are methods or reducing the risk of HAE attack in a human HAE patient who is in quiescent stage. Such a patient can be identified based on various factors, including history of HAE attack. An effective amount of one or more of the compounds can be given to the human patient via a suitable route, for example, those described herein. The compounds described herein may be used alone, or may be used in combination with other anti-HAE agents, for example, a C1 esterase inhibitor (e.g., Cinryze® or Berinert®), a PKa inhibitor (e.g., ecallantide or lanadelumab) or a bradykinin B2 receptor antagonist (e.g., Firazyr®).

In some embodiments, provided herein is prophylactic treatment of HAE in human patients having risk to HAE attacks with one or more of the compounds described herein. In some embodiments, patients suitable for prophylactic treatment of HAE are human subjects suffering from HAE (e.g., having history of HAE attacks). In some embodiments, patients suitable for such prophylactic treatment are human subjects where a physician determines a history of HAE attacks warrants a prophylactic approach (e.g., human subjects experiencing more than a particular average number of attacks over a time period, including by way of nonlimiting example, one, two, or more attacks per month). Alternatively, patients suitable for the prophylactic treatment may be human subjects having no HAE attack history but bearing one or more risk factors for HAE (e.g., family history, genetic defects in C1-INH gene, etc.) Such prophylactic treatment may involve the compounds described herein as the sole active agent, or involve additional anti-HAE agents, such as those described herein.

In certain embodiments, provided herein are methods for preventing or reducing edema in an eye of a subject (e.g., a human patient). In some examples, the human patient is a diabetic having, suspected of having, or at risk for diabetic macular edema (DME). DME is the proliferative form of diabetic retinopathy characterized by swelling of the retinal layers, neovascularization, vascular leak, and retinal thickening in diabetes mellitus due to leaking of fluid from blood vessels within the macula. To practice this method, an effective amount of one or more of the compounds described herein, or pharmaceutically acceptable salts thereof, may be delivered into the eye of the subject where treatment is needed. For example, the compound may be delivered topically, by intraocular injection, or intravitreal injection. A subject may be treated with the compound as described herein, either as the sole active agent, or in combination with another treatment for DME. Non-limiting examples of treatment for DME include laser photocoagulation, steroids, VEGF pathway targeting agents (e.g., Lucentis® (ranibizumab) or Eylea® (aflibercept)), and/or anti-PDGF agents.

In certain embodiments, the methods disclosed herein comprise administering to the subject an effective amount of a compound of Formulae (I)-(VI-b), or a pharmaceutically acceptable salt or composition thereof. In some embodiments, the effective amount is a therapeutically effective amount. In some embodiments, the effective amount is a prophylactically effective amount.

In certain embodiments, the subject being treated is an animal. The animal may be of either sex and may be at any stage of development. In certain embodiments, the subject is a mammal. In certain embodiments, the subject being treated is a human. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal such as a rodent (e.g., mouse, rat), dog, pig, or non-human primate. In certain embodiments, the animal is a genetically engineered animal. In certain embodiments, the animal is a transgenic animal.

Certain methods described herein may comprise administering one or more additional pharmaceutical agent(s) in combination with the compounds described herein. The additional pharmaceutical agent(s) may be administered at the same time as the compound of Formulae (I)-(VI-b), or at different times than the compound of Formulae (I)-(VI-b). For example, the compound of Formulae (I)-(VI-b) and any additional pharmaceutical agent(s) may be on the same dosing schedule or different dosing schedules. All or some doses of the compound of Formulae (I)-(VI-b) may be administered before all or some doses of an additional pharmaceutical agent, after all or some does an additional pharmaceutical agent, within a dosing schedule of an additional pharmaceutical agent, or a combination thereof. The timing of administration of the compound of Formulae (I)-(VI-b) and additional pharmaceutical agents may be different for different additional pharmaceutical agents.

In certain embodiments, the additional pharmaceutical agent comprises an agent useful in the treatment of an edema, such as HAE or DME. Examples of such agents are provided herein.

Also provided is use of a compound of the present disclosure for the manufacture of a medicament for a condition/disease disclosed herein.

In the context of this specification “comprising” is to be interpreted as “including”. Embodiments of the invention comprising certain features/elements are also intended to extend to alternative embodiments “consisting” or “consisting essentially” of the relevant elements/features. Where technically appropriate, embodiments of the invention may be combined.

Technical references such as patents and applications are incorporated herein by reference.

Any embodiments specifically and explicitly recited herein may form the basis of a disclaimer either alone or in combination with one or more further embodiments.

The background section of this specification contains relevant technical information and may be used as basis for amendment. Subject headings herein are employed to divide the document into sections and are not intended to be used to construe the meaning of the disclosure provided herein.

The present specification claims priority from U.S. Provisional Application No. 63/162,487 (filed Mar. 17, 2021) incorporated herein by reference. This application may be used as basis for corrections to the present specification, especially in respect of chemical structures disclosed therein.

### IV. EXAMPLES

In certain embodiments, the Examples describe compounds comprising one or more stereocenters, where a particular stereocenter is designated “S*” or “R*.” In both cases, the depiction of the “*” generally indicates that the exact configuration is unknown (e.g., for a compound with a single stereocenter, the depiction R*- or S*— indicates that either the R- or S-isomer was isolated, but the configuration at the stereocenter of the particular isomer isolated was not determined).

It will be appreciated that compounds described within the Examples may comprise more than one stereocenter. As described above, single stereochemical isomers as well as enantiomeric, diastereomeric, and geometric (or conformational) mixtures of the present compounds are within the scope of the invention. Within a particular compound name, where more than one “S*” or “R*” appear within a single pair of parentheses (e.g., “(1S*,2S*)”), it is understood that the S* and/or R* configurations are relative to each other. For example, a compound denoted “(1S*,2S*)-” or “(1R^{•},2R*)-” would be understood to refer specifically to either the “(1S,2S)—” or “(1R,2R)-” isomer, but not the “(1S,2R)-” or “(1R,2S)—” isomers. Furthermore, a compound denoted “rac-(1S*,2S*)-” or “rac-(1R^{•},2R*)-” would be understood to include a racemic mixture of the “(1S,2S)—” and “(1R,2R)-” isomers. Similarly, a compound denoted “(1S*,2R*)-” or “(1R^{•},2S*)-” would be understood to refer specifically to either the “(1R,2S)—” or “(1S,2R)-” isomer, but not the “(1S,2S)—” or “(1R,2R)-” isomers. In addition, a compound denoted “rac-(1R^{•},2S*)-” or “rac-(1S*,2R*)-” would be understood to include a racemic mixture of the “(1R,2S)—” and “(1S,2R)-” isomers.

In certain embodiments, the Examples include schemes that depict compounds with one or more stereocenters. In some embodiments, the symbol “&” followed by a number appears adjacent to a stereocenter. In such cases, it is understood to include a mixture of both configurations (e.g., R- and S—) at that position.

In some embodiments, the term “or” followed by a number appears adjacent to a stereocenter. In such cases, it is understood to denote either an “R-” or “S—” isomer, but the particular isomer was not determined.

In some embodiments, the numbering following the symbol “&” or term “or” refers to one stereocenter's relation to another stereocenter in that compound. For example, where two stereocenters in a compound are each denoted with the same number (e.g., two instances of “&1”), it is understood that the configurations are relative to each other (e.g., if the structure is drawn as (S,S) and both stereocenters are denoted “&1”, it is understood to include a mixture of the (S,S) and (R,R) isomers, but not the (S,R) or (R,S) isomers). However, where each stereocenter is denoted with a different number (e.g., one instance of “&1” and one instance of “&2”), it is understood that that the configurations may be independent to each other (e.g., if the structure is drawn (S,S) and one stereocenter is denoted “&1” and one is denoted “&2,” it is understood to include a mixture of the (S,S), (S,R), (R,S), and (R,R) isomers).

### Synthesis of Intermediates

### Synthesis of (1S,2S)-2-(3-chlorophenyl)cyclopropane-1-carboxylic acid (First Eluting Isomer)

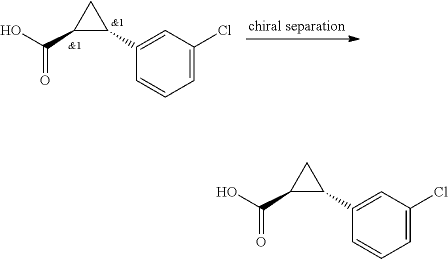 [SMILES: O=C(O)[C@H]1C[C@@H]1c1cccc(Cl)c1 ; O=C(O)[C@H]1C[C@@H]1c1cccc(Cl)c1]

rac-(1S*,2S*)-2-(3-chlorophenyl)cyclopropane-1-carboxylic acid (10 g) was separated using SFC (SFC80, Daicel CHIRALPAK AD-H 250 mm×20 mm I.D., 5 μm, CO₂/EtOH=86/14, 50 g/min, 35° C.) to give (1S,2S)-2-(3-chlorophenyl)cyclopropane-1-carboxylic acid (4.5 g, first eluting isomer, Rₜ=3.0 min, 99.9% e.e.) and (1R,2R)-2-(3-chlorophenyl)cyclopropane-1-carboxylic acid (4.3 g, second eluting isomer, Rₜ=4.0 min, 99.9% e.e.).

### Synthesis of (1S,2S)-2-(3-chlorophenyl)cyclopropane-1-carboxamide

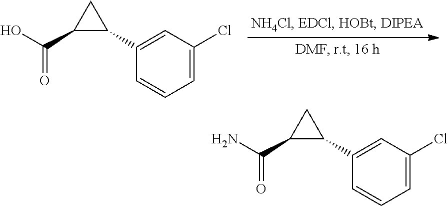 [SMILES: O=C(O)[C@H]1C[C@@H]1c1cccc(Cl)c1 ; NC(=O)[C@H]1C[C@@H]1c1cccc(Cl)c1]

To a solution of (1S,2S)-2-(3-chlorophenyl)cyclopropane-1-carboxylic acid (250 mg, 1.28 mmol) in DMF (4 mL) was added (NH₄)₂CO₃ (247 mg, 2.56 mmol), HOBt (260 mg, 1.92 mmol), EDCI (372 mg, 1.92 mmol) and DIPEA (498 mg, 3.84 mmol). After stirring at room temperature for 12 h, water (20 mL) was added then the reaction was extracted with EtOAc (5×50 mL). The combined organic layers were washed with brine (30 mL), dried over anhydrous Na₂SO₄, and concentrated in vacuo to give the crude, which was purified by silica gel column chromatography (eluent: DCM/MeOH=15/1) to give (1S,2S)-2-(3-chlorophenyl)cyclopropane-1-carboxamide (210 mg, 84%) as a white solid. ESI-MS [M+H]+: 196.1.

### Synthesis of (1S,2S)-2-(4-methylpyrimidin-2-yl)cyclopropane-1-carboxamide

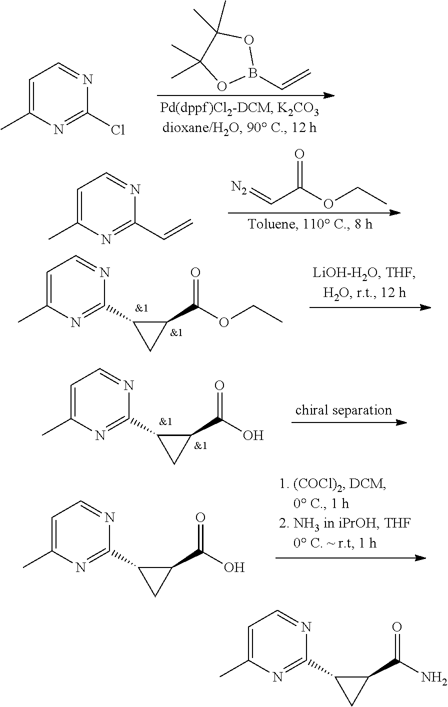

Synthesis of 4-methyl-2-vinylpyrimidine. A mixture of 2-chloro-4-methylpyrimidine (6.4 g, 50 mmol), 4,4,5,5-tetramethyl-2-vinyl-1,3,2-dioxaborolane (10 g, 65 mmol), Pd(dppf)C12-DCM (2 g, 2.5 mmol) and K₂CO₃ (17.25 g, 125 mmol) in dioxane (100 mL) and H₂O (5 ml) was stirred at 90° C. for 12 h. The reaction mixture was treated with H₂O (50 mL) and extracted with EA (50 ml×3). The combined organic layers were washed with brine (50 mL), dried over Na₂SO₄ and concentrated in vacuo (at 30° C.). The crude product was purified by silica column chromatography (PE/EtOAc=5/1) to give 4-methyl-2-vinylpyrimidine (4.8 g, 80%) as yellow oil. ESI-MS [M+H]⁺: 121.2.

Synthesis of rac-ethyl (1S* 2S*)-2-(4-methylpyrimidin-2 yl)cyclopropane-1-carboxylate. A solution of 4-methyl-2-vinylpyrimidine (4.8 g, 40 mmol) and ethyl 2-diazoacetate (9.12 g, 80 mmol) in toluene (70 mL) was refluxed at 110° C. for 8 h. The reaction was concentrated in vacuo and the crude product was purified by silica gel column (PE/EA=5/1) to give rac-ethyl (1S*, 2S*)-2-(4-methylpyrimidin-2-yl)cyclopropane-1-carboxylate (3.6 g, 43.7%) as yellow oil. ESI-MS [M+H]⁺: 207.2.

Synthesis of rac-(1S*,2S*)-2-(4-methylpyrimidin-2yl)cyclopropane-1-carboxylic acid. A mixture of rac-ethyl (1S*, 2S*)-2-(4-methylpyrimidin-2-yl)cyclopropane-1-carboxylate (3.6 g, 17.5 mmol) and LiOH—OH (1.4 g, 35 mmol) in THF/H₂O (20 mL/10 mL) was stirred at room temperature for 12 h. The reaction was concentrated in vacuo to remove THF and the pH of the residue was adjusted to 3 by HCl (2N). The white solid was precipitated, and mixture was filtered, dried to give rac-(1S*,2S*)-2-(4-methylpyrimidin-2-yl)cyclopropane-1-carboxylic acid (2.4 g, 77%) as a white solid. ESI-MS [M+H]⁺: 179.2.

Synthesis of rac-(1S*,2S*)-2-(4-methylpyrimidin-2-yl)cyclopropane-carboxamide. To a solution of rac-(1S*,2S*)-2-(4-methylpyrimidin-2-yl)cyclopropane-1-carboxylic acid (1.0 g, 5.6 mmol) in dry DCM (20 mL) was added (COCl)₂ (1.41 g, 11.2 mmol) at 0° C. slowly and was stirred at 0° C. for 1 h. The reaction mixture was concentrated in vacuo and the resulting acid chloride was dissolved in dry THF (20 mL), cooled to 0° C. and NH₃ (20 mL, 2 M solution in iPrOH) was added. The resulting solution was stirred at room temperature for 1 h and concentrated in vacuo to give crude, which was purified with silica gel chromatography (eluent: DCM/MeOH=20/1) to give rac-(1S*,2S*)-2-(4-methylpyrimidin-2-yl)cyclopropane-1-carboxamide (800 mg, 80%) as a yellow solid. ESI-MS [M+H]⁺: 178.1.

Synthesis of (1S,2S)-2-(4-methylpyrimidin-2-yl)cyclopropane-1-carboxylic acid. The racemic mixture form of rac-(1S*,2S*)-2-(4-methylpyrimidin-2-yl)cyclopropane-1-carboxylic acid (2.4 g) was chiral separated with SFC to give (1S,2S)-2-(4-methylpyrimidin-2-yl)cyclopropane-1-carboxylic acid (1.1 g, 45.8%) as a white solid. ESI-MS [M+H]⁺: 179.1.

Synthesis of (1S,2S)-2-(4-methylpyrimidin-2-yl)cyclopropane-1-carboxamide. To a solution of (1S,2S)-2-(4-methylpyrimidin-2-yl)cyclopropane-1-carboxylic acid (1.1 g, 6.1 mmol) in dry DCM (20 mL) was added (COCl)₂ (1.56 g, 12.3 mmol) at 0° C. slowly and was stirred at 0° C. for another 1 h. The reaction mixture was concentrated in vacuo and the resulting acid chloride was dissolved in dry THF (20 mL), cooled to 0° C. and then added NH₃ (20 mL, 2 M solution in iPrOH). The resulting solution was stirred at room temperature for another 1 h and concentrated in vacuo to give crude, which was purified with silica gel chromatography (eluent: DCM/MeOH=20/1) to furnish (1S,2S)-2-(4-methylpyrimidin-2-yl)cyclopropane-1-carboxamide (900 mg, 81.2%) as a yellow solid. ESI-MS [M+H]⁺: 178.1.

### Synthesis of ethyl (1S,2S)-2-(4-methylpyrimidin-2-yl)cyclopropane-1-carboxylate

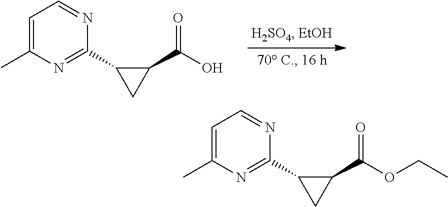

To a solution of (1S,2S)-2-(4-methylpyrimidin-2-yl)cyclopropane-1-carboxylic acid ((500 mg, 2.8 mmol) in EtOH (10 mL) was added H₂SO₄ (conc. 0.5 mL). The mixture was stirred at 70° C. for 16 h, then cooled to room temperature, diluted with water (40 mL) and the pH of the solution was adjusted to 7 by NaHCO₃ (sat. aq., 20 mL). The mixture was extracted with EtOAc (40 mL×3). The combined organic layers were washed with brine (50 mL), dried over Na₂SO₄ and concentrated in vacuo to give ethyl (1S,2S)-2-(4-methylpyrimidin-2-yl)cyclopropane-1-carboxylate (500 mg, crude) which was used in the next step without purification. ESI-MS [M+H]⁺: 207.2

### Synthesis of rac-(1S*,2S*)-2-(4-chloropyridin-2-yl)cyclopropane-1-carboxamide

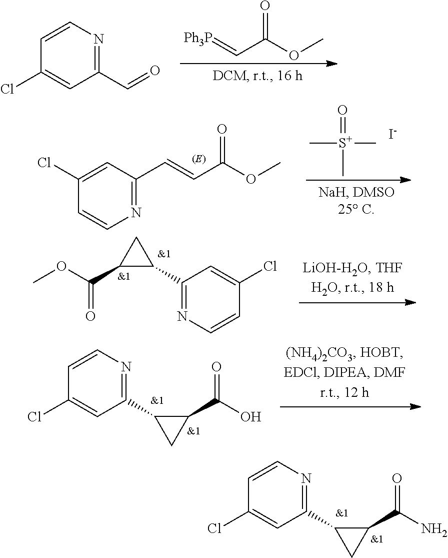

Synthesis of methyl (E)-3-(4-chloropyridin-2-yl)acrylate. A mixture of 4-chloropicolinaldehyde (2 g, 14.2 mmol) and methyl 2-(triphenyl-15-phosphaneylidene)acetate (2.4 g, 14.2 mmol) in DCM (30 mL) was stirred at room temperature for 16 h. The reaction was quenched with water (100 mL) then extracted with DCM (3×50 mL). The combined organic layers were washed with brine (30 mL), dried over Na₂SO₄ and concentrated in vacuo. The residue was purified by silica gel column chromatography (eluent: EtOAc/PE from 0 to 20%) to afford methyl (E)-3-(4-chloropyridin-2-yl)acrylate (2.5 g, 89% yield) as a white solid. ESI-MS [M+H]⁺: 198.1.

Synthesis of rac-methyl (1S*,2S*)-2-(4-chloropyridin-2yl)cyclopropane-1-carboxylate. To a mixture of trimethylsulfoxonium iodide (8.4 g, 38.1 mmol) in DMSO (50 mL) was added NaH (1.5 g, 38.1 mmol) and the mixture was stirred at room temperature for 2 h. Methyl (E)-3-(4-chloropyridin-2-yl)acrylate (2.5 g, 12.7 mmol) in DMSO (10 mL) was added and the resulting mixture was stirred at room temperature for 16 h. The reaction was quenched with saturated NH₄Cl solution (100 mL) then extracted with EtOAc (3×100 mL). The combined organic layers were washed with brine (100 mL), dried over Na₂SO₄ and concentrated in vacuo. The residue was purified by silica gel column chromatography (eluent: EtOAc/PE from 0 to 20%) to afford rac-methyl (1S*,2S*)-2-(4-chloropyridin-2-yl)cyclopropane-1-carboxylate (0.7 g, 26% yield) as a yellow oil. ESI-MS [M+H]⁺: 212.2.

Synthesis of rac-(1S* 2S*)-2-(4-chloropyridin-2yl)cyclopropane-carboxylic acid. A mixture of rac-methyl (1S*,2S*)-2-(4-chloropyridin-2-yl)cyclopropane-1-carboxylate (340 mg, 1.6 mmol) and LiOH—H₂O (135 mg, 3.2 mmol) in THF/H2O (5 mL/5 mL) was stirred at room temperature for 18 h. The pH of the reaction mixture was adjusted to pH˜3 with 1N HCl and the mixture was extracted with IPA/CHCl₃ (3/1, 5×30 mL). The combined organic layers were washed with brine (30 mL), dried over Na₂SO₄ and concentrated in vacuo to afford rac-(1S*,2S*)-2-(4-chloropyridin-2-yl)cyclopropane-1-carboxylic acid (300 mg, crude) as an off-white solid, which was used in the next step directly. ESI-MS [M+H]⁺: 198.2.

Synthesis of rac-(1S*,2S*)-2-(4-chloropyridin-2yl)cyclopropane-1-carboxamide. A mixture of rac-(1S*,2S*)-2-(4-chloropyridin-2-yl)cyclopropane-1-carboxylic acid (300 mg, 1.52 mmol), (NH₄)₂CO₃ (670 mg, 7.6 mmol), HOBt (410 mg, 3.04 mmol), EDCI (578 mg, 3.04 mmol) and DIEA (588 mg, 4.56 mmol) in DMF (5 mL) was stirred at room temperature for 12 h. The mixture was quenched with water (150 mL) and extracted with EtOAc (5×50 mL). The combined organic layers were washed with brine (50 mL), dried over Na₂SO₄ and concentrated in vacuo. The residue was purified by silica gel column chromatography (EtOAc/PE from 0 to 50%) to rac-(1S*,2S*)-2-(4-chloropyridin-2-yl)cyclopropane-1-carboxamide (220 mg, 73%) as an off-white solid. ESI-MS [M+H]⁺: 197.2

The mixture was separated using SFC (SFC80, Daicel CHIRALPAK AD-H, 250 mm×20 mm I.D., 5 μm, CO₂/MEOH=74/26, 50 g/min, 35° C.) to give two enantiomers: (1S,2S)-2-(4-chloropyridin-2-yl)cyclopropane-1-carboxamide (0.1 g, first eluting isomer, Rₜ=2.6 min, 99.9% e.e) and (1R,2R)-2-(4-chloropyridin-2-yl)cyclopropane-1-carboxamide (0.1 g, second eluting isomer, Rₜ=5.5 min, 99.9% e.e)

The compound in Table 1 was prepared in a similar manner to rac-(1S*,2S*)-2-(4-chloropyridin-2-yl)cyclopropane-1-carboxamide starting from 2-bromo-5-chlorobenzaldehyde.

TABLE 1

| Structure | Analytical data |
|---|---|
| 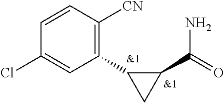 | ESI-MS [M + H]+: 221.1. |
| rac-(1S*,2S*)-2-(5-chloro-2-cyanophenyl)cyclopropane-1-carboxamide | |

### Synthesis of rac-(1R*,2R*)-6′-chloro-2′,3′-dihydrospiro[cyclopropane-1,1′-indene]-2-carboxamide

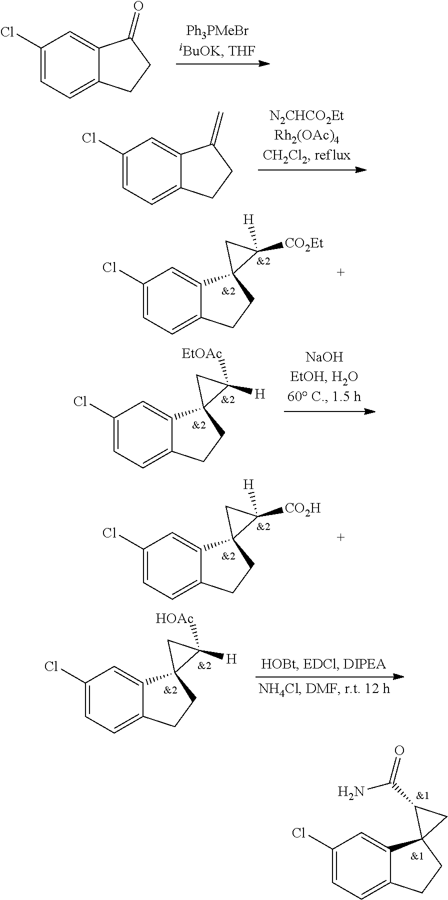

Synthesis of 6-chloro-1-methylene-2,3-dihydro-1H-indene. A solution of potassium tert-butoxide (97.0 mL, 1.0 M in THF, 97.0 mmol) was added to a suspension of methyltriphenylphosphonium bromide (34.7 g, 97.1 mmol) in anhydrous THF (150 mL) under nitrogen at 0° C. The bright yellow suspension was stirred for 1 h and a solution of 6-chloroindan-1-one (8.09 g, 48.6 mmol) in anhydrous THF (75 mL) was added over 5 min. Once the addition was complete the cooling bath was removed. After 1 h, saturated aqueous ammonium chloride (100 mL) was added slowly. After being stirred for 10 min, the THF was removed in vacuo, and the residue was diluted with ethyl acetate (150 mL) and water (100 mL). The layers were separated, and the organic phase washed with brine, dried (MgSO₄) and concentrated in vacuo onto silica gel (50 g). This material was applied to the top of a chromatography column (200 g silica gel) which was eluted with heptane to afford 6-chloro-1-methylene-2,3-dihydro-1H-indene (7.24 g, 90%) as a light-yellow oil. ¹H NMR (400 MHz, CDCl₃, ppm): δ 7.44 (s, 1H), 7.18-7.16 (m, 2H), 5.44 (t, J 2 Hz, 1H), 5.07 (t, J 2 Hz, 1H), 2.95-2.92 (m, 2H), 2.84-2.80 (m, 2H).

Synthesis of ethyl (1S*,2S*)-6′-chloro-2′,3′-dihydrospiro[cyclopropane-1,1′-indene]-2-carboxylate and ethyl (1S*,2R*)-6′-chloro-2′,3′-dihydrospiro[cyclopropane-1,1′-indene]-2-carboxylate. A solution of ethyl diazoacetate (85%, 10.0 g, 104 mmol) in anhydrous DCM (20 mL) was added via syringe pump over 5 h to a stirred solution of 6-chloro-1-methylene-2,3-dihydro-1H-indene (5.69 g, 34.6 mmol) and rhodium acetate (153 mg, 0.346 mmol) in anhydrous DCM (80 mL) under nitrogen at reflux. The dark green-blue mixture was concentrated in vacuo, dissolved in toluene and applied to the top of a silica gel column (500 g), which was eluted with 1-4% methyl tert-butyl ether in heptane in 1% increments to give a mixture of ethyl (1S*,2S*)-6′-chloro-2′,3′-dihydrospiro[cyclopropane-1,1′-indene]-2-carboxylate and ethyl (1S*,2R*)-6′-chloro-2′,3′-dihydrospiro[cyclopropane-1,1′-indene]-2-carboxylates (ratio 6:5, 3.932 g, 45%). Purification of several aliquots (10×7 mg) of the isomeric mixture by preparative hplc was performed:

First eluted was a colourless oil (10 mg, RT=5.6 min, minor isomer). This was assigned as the (1R^{•},2S*) by NOE experiments. ¹H NMR (400 MHz, CDCl₃, ppm): δ 7.18 (s, 1H), 7.10 (m, 2H), 4.06 (dq, J 11, 7 Hz, 1H), 3.99 (dq, J 11, 7 Hz, 1H), 3.05 (ddd, J 8.2, 10, 16 Hz, 1H), 2.86 (ddd, J 1.5, 8.9, 16 Hz, 1H), 2.39 (ddd, J 8.9, 10, 13 Hz, 1H), 2.10 (dd, J 6.4, 8 Hz, 1H), 1.92 (ddd, J 1.5, 8.2, 13 Hz, 1H), 1.84 (dd, J 5.6, 6.4 Hz, 1H), 1.41 (dd, J 5.6, 8 Hz, 1H), 1.16 (t, J 7 Hz, 3H).

Second eluted was a colourless oil (16 mg, RT=6.0 min, major isomer). This was assigned as the (1R^{•},2R*) by NOE experiments. ¹H NMR (400 MHz, CDCl₃, ppm): δ 7.10 (m, 2H), 6.65 (s, 1H), 4.15 (m, 2H), 2.96 (m, 2H), 2.27 (m, 2H), 2.00 (dd, J 6, 8.5 Hz, 1H), 1.65 (dd, J 5, 6 Hz, 1H), 1.39 (dd, J 5, 8.5 Hz, 1H), 1.25 (t, J 7.2 Hz, 3H).

Synthesis of (1S*,2S*)-6′-chloro-2′,3′-dihydrospiro[cyclopropane-1,1′-indene]-2-carboxylic acid and (1S*,2R*)-6′-chloro-2′,3′-dihydrospiro[cyclopropane-1,1′-indene]-2-carboxylic acid. A solution of sodium hydroxide (1.88 g, 46.9 mmol) in water (23 mL) was treated with a solution of a mixture of ethyl (1S*,2S*)-6′-chloro-2′,3′-dihydrospiro[cyclopropane-1,1′-indene]-2-carboxylate and ethyl (1S*,2R*)-6′-chloro-2′,3′-dihydrospiro[cyclopropane-1,1′-indene]-2-carboxylates (ratio 6:5, 3.92 g, 15.6 mmol) in ethanol (75 mL) under nitrogen. The mixture was heated at 60° C. for 90 min then was cooled to room temperature, concentrated in vacuo, and the residue was diluted with water (70 mL). Hydrochloric acid (2 M) was added to give pH 1 which precipitated a gummy solid. The gum was extracted into ethyl acetate (3×70 mL), which was dried (Na₂SO₄) and concentrated in vacuo to give a pale brown solid. Recrystallisation from a mixture of boiling heptane (70 mL) and ethyl acetate (20 mL) gave (1S*,2S*)-6′-chloro-2′,3′-dihydrospiro[cyclopropane-1,1′-indene]-2-carboxylic acid (1169 mg, 30%), as colourless needles. ESI_MS [M−H]⁻ 221/223, ¹H NMR (400 MHz, DMSO, ppm): δ 12.28 (br s, 1H), 7.22 (d, J 8 Hz, 1H), 7.17 (dd, J 8, 1.7 Hz, 1H), 6.98 (d, J 1.7 Hz, 1H), 2.99-2.86 (m, 2H), 2.24-2.11 (m, 2H), 2.00 (dd, J 6, 8.5 Hz, 1H), 1.48 (dd, J 5, 8.5 Hz, 1H), 1.44 (dd, J 5, 6 Hz, 1H).

Synthesis of rac-(1S*,2S*)-6′-chloro-2′,3′-dihydrospiro[cyclopropane-1,1′-indene]-2-carboxamide. A mixture of rac-(1S*,2S*)-6′-chloro-2′,3′-dihydrospiro[cyclopropane-1,1′-indene]-2-carboxylic acid (230 mg, 1.04 mmol), NH₄Cl (220 mg, 4.14 mmol), HOBt (351 mg, 2.6 mmol), EDCI (500 mg, 2.6 mmol ) and DIPEA (671 mg, 5.2 mmol) in DMF (15 mL) was stirred at room temperature for 12 h under N₂. The reaction was quenched with water (30 mL) then extracted with EtOAc (3×30 mL). The combined organic layers were washed with brine (50 mL), dried over Na₂SO₄, then concentrated in vacuo to give the crude, which was triturated with DCM (10 mL) to give rac-(1S*,2S*)-6′-chloro-2′,3′-dihydrospiro[cyclopropane-1,1′-indene]-2-carboxamide (180 mg, 78%) as a white solid. ESI-MS [M+H]⁺: 222.1.

### Synthesis of rac-ethyl (1S*,2S*-2-(4-methylpyridin-2-yl)cyclopropane-1-carboxylate

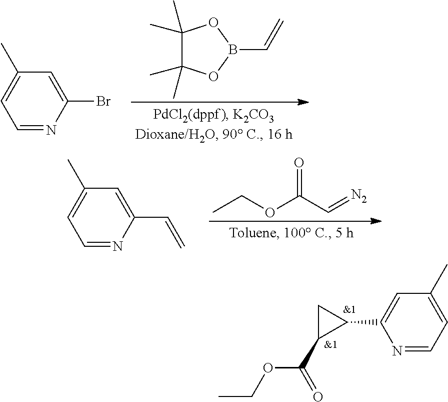

Synthesis of 4-methyl-2-vinylpyridine. A mixture of 2-bromo-4-methylpyridine (3.3 mL, 5 g, 29.06 mmol), 4,4,5,5-tetramethyl-2-vinyl-1,3,2-dioxaborolane (5.9 mL, 5.37 g, 34.87 mmol) and K₂CO₃ (12.05 g, 87.18 mmol) in 1,4 -dioxane/H₂O (40 mL/4 mL) was added Pd(dppf)C12 (2.13 g, 2.91 mmol) at room temperature. The mixture was stirred at 90° C. for 16 h under N₂. After cooling to 25° C., water (50 mL) was added and the mixture was extracted with DCM (3×60). The combined organic layers were washed with brine (60 mL), dried over anhydrous Na₂SO₄ and concentrated in vacuo to give the crude product, which was purified by flash chromatography (eluent: PE/EtOAc=6/1) to give 4-methyl-2-vinylpyridine (3.277 g, 95%) as a yellow oil. ESI-MS [M+H]⁺: 120.1.

Synthesis of rac-ethyl (1S*,2S*)-2-(4-methylpyridin-2-yl)cyclopropane-1-carboxylate. A solution of 4-methyl-2-vinylpyridine (1.2 g, 10.07 mmol) in toluene (50 mL) was added ethyl 2-diazoacetate (3.447 g, 30.21 mmol) at room temperature. The mixture was stirred at 100° C. for 5 h under N₂, cooled to room temperature and concentrated in vacuo to give the crude product, which was purified by flash chromatography (eluent: PE/EtOAc=10/1) to give rac-ethyl (1S*,2S*)-2-(4-methylpyridin-2-yl)cyclopropane-1-carboxylate (1.473 g, 71%) as a yellow oil. ESI-MS [M+H]⁺: 206.1.

Synthesis of rac-ethyl (1S*,2S*)-2-(3-fluoro-4-methylpyridin-2-yl)cyclopropane-1-carboxylate

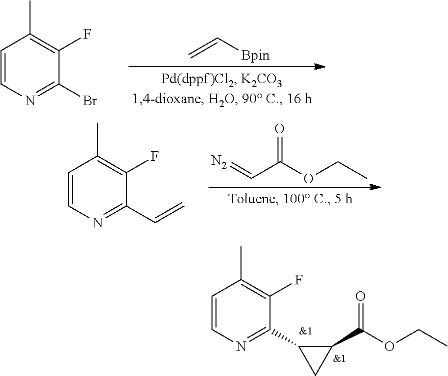

Synthesis of 3-fluoro-4-methyl-2-vinylpyridine. To a mixture of 2-bromo-3-fluoro-4-methylpyridine (1 g, 5.3 mmol), 4,4,5,5-tetramethyl-2-vinyl-1,3,2-dioxaborolane (978 mg, 6.3 mmol), K₂CO₃ (2.2 g, 15.9 mmol) in dioxane (20 mL) and water (5 ml) was added Pd(dppf)C12 (388 mg, 0.53 mmol). The mixture was stirred at 90° C. for 16 h, cooled to rt then water (10 mL) was added. The mixture was extracted with EtOAc (20 mL×3). The combined organic layers were concentrated in vacuo to give the crude product, which was purified by silica gel chromatography (eluent: PE/EA=6/1) to give 3-fluoro-4-methyl-2-vinylpyridine (300 mg, 41%) as a yellow oil. ESI-MS [M+H]⁺: 138.2.

Synthesis of rac-ethyl (1S*,2S*)-2-(3-fluoro-4-methylpyridin-2yl)cyclopropane-1-carboxylate. To a mixture of 3-fluoro-4-methyl-2-vinylpyridine (300 mg, 2.19 mmol) in PhMe (5 mL) was added ethyl 2-diazoacetate (749 mg, 6.57 mmol). The mixture was stirred at 100° C. for 6 h under N₂ then concentrated in vacuo to give the crude product, which was purified by preparative TLC (PE/EtOAc=5/1) to give rac-ethyl (1S*,2S*)-2-(3-fluoro-4-methylpyridin-2-yl)cyclopropane-1-carboxylate (140 mg, 29%) as a yellow solid. ESI-MS [M+H]⁺: 244.2.

### Synthesis of (1S,2S)—N-(6-bromopyrimidin-4-yl)-2-(3-chlorophenyl)cyclopropane-1-carboxamide

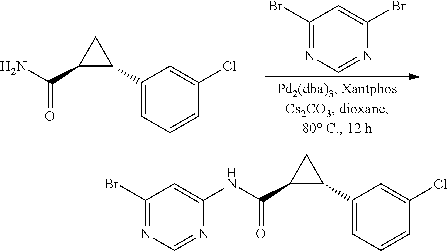 [SMILES: NC(=O)[C@H]1C[C@@H]1c1cccc(Cl)c1 ; Brc1cc(Br)ncn1 ; O=C(Nc1cc(Br)ncn1)[C@H]1C[C@@H]1c1cccc(Cl)c1]

To a solution of 4,6-dibromopyrimidine (474 mg, 2 mmol) in dioxane (10 mL) was added (1S,2S)-2-(3-chlorophenyl)cyclopropane-1-carboxamide (195 mg, 1 mmol), Pd₂(dba)₃ (9 mg, 0.01 mmol), Xantphos (12 mg, 0.02 mmol) and Cs₂CO₃ (978 mg, 3 mmol). Then the mixture was stirred for 12 h at 80° C. under N₂ then cooled to room temperature. Water (30 mL) was added and the mixture extracted with EtOAc (30 mL×3). The combined organic layers were concentrated in vacuo to give the crude, which was purified by preparative TLC (DCM/MeOH=20/1) to give (1S,2S)—N-(6-bromopyrimidin-4-yl)-2-(3-chlorophenyl)cyclopropane-1-carboxamide as a white solid (200 mg, yield: 57%). ESI-MS [M+H]⁺: 352.2.

The compounds in Table 2 were prepared in a similar manner to (1S,2S)—N-(6-bromopyrimidin-4-yl)-2-(3-chlorophenyl)cyclopropane-1-carboxamide from the indicated starting materials

TABLE 2

| Structure | Starting materials | Analytical data |
|---|---|---|
| 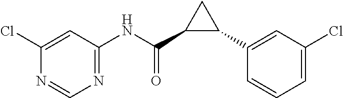 [SMILES: O=C(Nc1cc(Cl)ncn1)[C@H]1C[C@@H]1c1cccc(Cl)c1] | 4,6-dichloro-pyrimidine and (1S,2S)-2-(3- chlorophenyl)cyclopropane- 1-carboxamide | ESI-MS [M + H]+: 308.2. |
| (1S,2S)-2-(3-chlorophenyl)-N-(6- | | |
| chloropyrimidin-4-yl)cyclopropane-1- | | |
| carboxamide | | |
| 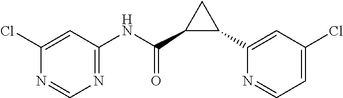 [SMILES: O=C(Nc1cc(Cl)ncn1)[C@H]1C[C@@H]1c1cc(Cl)ccn1] | 4,6-dichloro-pyrimidine and (1S,2S)-2-(4-chloropyridin-2- yl)cyclopropane-1- carboxamide | ESI-MS [M + H]+: 309.2 |
| (1S,2S)-2-(4-chloropyridin-2-yl)-N-(6- | | |
| chloropyrimidin-4-yl)cyclopropane-1- | | |
| carboxamide | | |

### Synthesis of rac-(1S*,2S*)—N-(6-chloropyrimidin-4-yl)-2-(4-methylpyridin-2-yl)cyclopropane-1-carboxamide

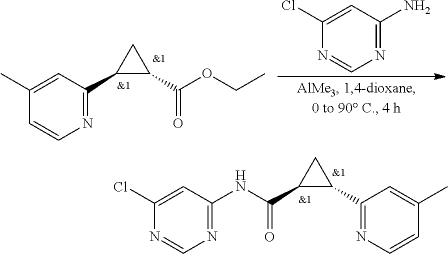 [SMILES: CCOC(=O)[C@H]1C[C@@H]1c1cc(C)ccn1 ; Nc1cc(Cl)ncn1 ; Cc1ccnc([C@H]2C[C@@H]2C(=O)Nc2cc(Cl)ncn2)c1]

To a mixture of 6-chloropyrimidin-4-amine (95 mg, 0.72 mmol) in 1,4-dioxane (5 mL) was added AlMe₃ (2 M solution in toluene, 0.36 mL, 0.72 mmol) at 0° C. The reaction was stirred at 35° C. for 1 h. Then a solution of rac-ethyl (1S*,2S*)-2-(4-methylpyridin-2-yl)cyclopropane-1-carboxylate (50 mg, 0.24 mmol) in 1,4-dioxane (1 mL) was added and the mixture was stirred at 90° ° C. for 4 h. The reaction mixture was cooled to room temperature. The mixture was quenched with water (30 mL) and extracted with EtOAc (20 mL×3). The combined organic layers were washed with brine (30 mL), dried over anhydrous Na₂SO₄, and concentrated in vacuo to give the crude product, which was purified by column chromatography (eluent: DCM/MeOH=20/1) to give rac-(1S*,2S*)—N-(6-chloropyrimidin-4-yl)-2-(4-methylpyridin-2-yl)cyclopropane-1-carboxamide (65 mg, 92%) as yellow solid. ESI-MS [M+H]+: 289.1.

The compounds in Table 3 were prepared in a similar manner to rac-(1S*,2S*)—N-(6-chloropyrimidin-4-yl)-2-(4-methylpyridin-2-yl)cyclopropane-1-carboxamide from the indicated starting materials

TABLE 3

| Structure | Starting materials | Analytical data |
|---|---|---|
| 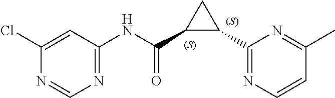 | 6-chloropyrimidin- 4-amine and ethyl (1S,2S)-2-(4- methylpyrimidin-2- yl)cyclopropane-1- carboxylate | ESI-MS [M + H]+: 290.2. |
| (1S,2S)-N-(6-chloropyrimidin-4-yl)-2-(4- | | |
| methylpyrimidin-2-yl)cyclopropane-1-carboxamide | | |
| 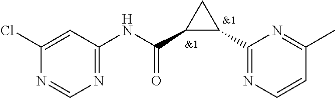 [SMILES: Cc1ccnc([C@H]2C[C@@H]2C(=O)Nc2cc(Cl)ncn2)n1] | 6-chloropyrimidin- 4-amine and rac- ethyl (1S*, 2S*)-2- (4-methylpyrimidin- 2-yl)cyclopropane- 1-carboxylate | ESI-MS [M + H]+: 290.2. |
| rac-(1S*,2S*)-N-(6-chloropyrimidin-4-yl)-2-(4- | | |
| methylpyrimidin-2-yl)cyclopropane-1-carboxamide | | |
| 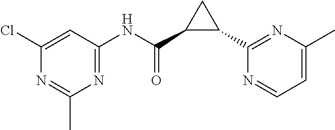 [SMILES: Cc1ccnc([C@H]2C[C@@H]2C(=O)Nc2cc(Cl)nc(C)n2)n1] | 2-methyl-6- chloropyrimidin-4- amine and ethyl (1S,2S)-2-(4- methylpyrimidin-2- yl)cyclopropane-1- carboxylate | ESI-MS [M + H]+: 304.2 |
| (1S,2S)-N-(6-chloro-2-methylpyrimidin-4-yl)-2-(4- | | |
| methylpyrimidin-2-yl)cyclopropane-1-carboxamide | | |
| 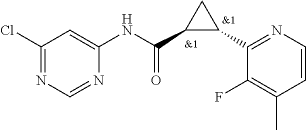 [SMILES: Cc1ccnc([C@H]2C[C@@H]2C(=O)Nc2cc(Cl)ncn2)c1F] | 6-chloropyrimidin- 4-amine and rac- ethyl (1S*,2S*)-2- (3-fluoro-4- methylpyridin-2- yl)cyclopropane-1- carboxylate | ESI-MS [M + H]+: 307.1. |
| rac-(1S*,2S*)-N-(6-chloropyrimidin-4-yl)-2-(3-fluoro- | | |
| 4-methylpyridin-2-yl)cyclopropane-1-carboxamide | | |

### Synthesis of (1S,2S)—N-(4-chloro-5-fluoropyrimidin-2-yl)-2-(4-methylpyrimidin-2-yl)cyclopropane-1-carboxamide

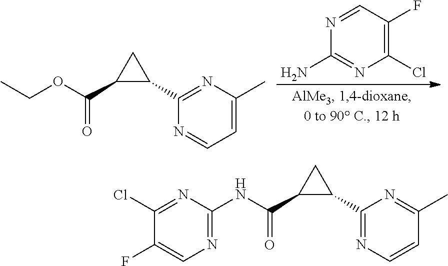

To a mixture of 4-chloro-5-fluoropyrimidin-2-amine (106 mg, 0.72 mmol) in 1,4-dioxane (5 mL) was added AlMe₃ (2 M solution in toluene, 0.36 mL, 0.72 mmol) at 0° C. The reaction was stirred at 35° C. for 1 h. Then a solution of ethyl (1S,2S)-2-(4-methylpyrimidin-2-yl)cyclopropane-1-carboxylate (50 mg, 0.24 mmol) in 1,4-dioxane (1 mL) was added and the mixture was stirred at 90° C. for 12 h. The reaction mixture was cooled to room temperature. The mixture was quenched with water (30 mL) and extracted with EtOAc (30 mL×3). The combined organic layers were washed with brine (30 mL), dried over anhydrous Na₂SO₄, and concentrated in vacuo to give the crude product, which was purified by column chromatography (eluent: DCM/MeOH=20/1) to give (1S,2S)—N-(4-chloro-5-fluoropyrimidin-2-yl)-2-(4-methylpyrimidin-2-yl)cyclopropane-1-carboxamide (60 mg, 81%) as yellow solid. ESI-MS [M+H]⁺: 308.1.

### Synthesis of (1S,2S)—N-(5-chloropyridazin-3-yl)-2-(4-methylpyrimidin-2-yl)cyclopropane-1-carboxamide

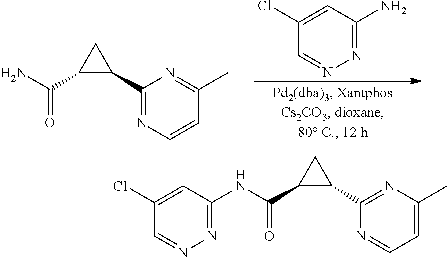 [SMILES: Cc1ccnc([C@@H]2C[C@H]2C(N)=O)n1 ; Nc1cc(Cl)cnn1 ; Cc1ccnc([C@H]2C[C@@H]2C(=O)Nc2cc(Cl)cnn2)n1]

To a solution of 3,5-dichloropyridazine (298 mg, 2 mmol) in dioxane (10 mL) was added (1S,2S)-2-(4-methylpyrimidin-2-yl)cyclopropane-1-carboxamide (177 mg, 1 mmol), Pd₂(dba)₃ (9 mg, 0.01 mmol), Xantphos (12 mg, 0.02 mmol) and Cs₂CO₃ (978 mg, 3 mmol). Then the mixture was stirred for 12 h at 80° C. under N₂ then cooled to room temperature. Water (30 mL) was added and the mixture extracted with EtOAc (30 mL×3). The combined organic layers were concentrated in vacuo to give the crude, which was purified by preparative TLC (DCM/MeOH=20/1) to give (1S,2S)-2-(3-chlorophenyl)-N-(6-chloropyrimidin-4-yl)cyclopropane-1-carboxamide as a white solid (203 mg, yield: 70%). ESI-MS [M+H]⁺: 290.1.

### Synthesis of (4-((JH-pyrazol-1-yl)methyl)phenyl)methanol

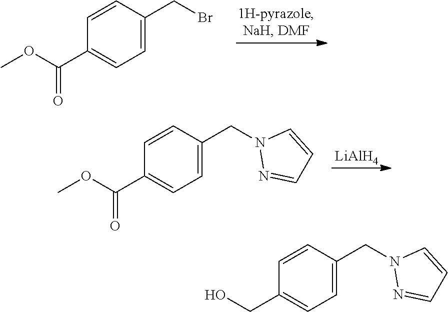

Synthesis of methyl 4-((1H-pyrazol-1-yl)methyl)benzoate. NaH (60% in mineral oil, 4.8 g, 121 mmol) was added portion-wise to a solution of 1H-pyrazole (7.5 g, 110 mmol) and methyl 4-(bromomethyl)benzoate (25.2 g, 110 mmol) in DMF (100 mL) at 0° C. under a N₂ atmosphere. The mixture was stirred at 0° C. until gas evolution had ceased. The mixture was allowed to warm to room temperature and more DMF (20 mL) was added to aid stirring. The mixture was stirred overnight at room temperature. The mixture was poured onto ice-water (400 mL) and extracted with EtOAc (3×400 mL). The combined organic extracts were dried over MgSO₄ then concentrated in vacuo. The resulting residue was washed with isohexane (×3) and dried in a vacuum oven to give the desired product (18.5 g, 77%). ¹H NMR (400 MHz, CDCl₃) δ 8.01-7.98 (m, 2H), 7.58-7.56 (m, 1H), 7.42-7.40 (m, 1H), 7.24-7.21 (m, 2H), 6.31 (t, J=2.1 Hz, 1H), 5.38 (s, 2H), 3.90 (s, 3H).

Synthesis of (4-((1H-pyrazol-1-yl)methyl)phenyl)methanol. LiAlH₄ (1.0 g, 27.6 mmol, crushed pellets) was added to a solution of methyl 4-((1H-pyrazol-1-yl)methyl)benzoate (5 g, 23.0 mmol) in THF (50 mL) at 0° C. under a N₂ atmosphere. After 45 min, the ice bath was removed, and the mixture was stirred for 16 h at room temperature. The mixture was quenched at 0° C. using water (4.4 mL) and NaOH (20% aq., 1.1 mL) and Et₂O (100 mL) was added. The mixture was stirred at 0° C. for 30 min, and then room temperature for 3 h. MgSO₄ was added and stirring was continued for a further 15 min. The mixture was filtered, and the filtrate was concentrated in vacuo to give the desired product (4.23 g, 98%) as a colourless oil. ¹H NMR (400 MHz, CDCl₃) δ 7.55-7.52 (m, 1H), 7.39-7.31 (m, 3H), 7.23-7.17 (m, 2H), 6.29-6.26 (m, 1H), 5.32-5.30 (m, 2H), 4.69-4.66 (m, 2H), exchangeable OH signal not observed.

### Synthesis of 1-(4-(chloromethyl)benzyl)-1H-pyrazole

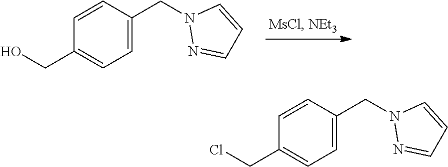 [SMILES: OCc1ccc(Cn2cccn2)cc1 ; ClCc1ccc(Cn2cccn2)cc1]

(4-((1H-pyrazol-1-yl)methyl)phenyl)methanol (1.5 g, 8.0 mmol) was dissolved in DCM (25 g) under a N₂ atmosphere, and NEt₃ (1.7 mL, 12.0 mmol) was added. The reaction mixture was cooled to 0° C. and MsCl (0.7 mL, 8.8 mmol) was added dropwise. The reaction was stirred at 0° C. for 1 h, then at room temperature for 4 h. The reaction mixture was washed with KHSO₄ (10% aq.), water and brine. The organic phase was collected, passed through a hydrophobic frit, and concentrated in vacuo. The crude residue was purified by silica gel chromatography, eluting with a gradient of 0-15% EtOAc in isohexane to give the desired product (840 mg, 51%). ESI-MS (M+H)+: 207, ¹H NMR (400 MHz, DMSO) δ 7.84-7.83 (m, 1H), 7.47-7.47 (m, 1H), 7.42-7.39 (m, 2H), 7.23-7.19 (m, 2H), 6.28 (t, J=2.1 Hz, 1H), 5.35 (s, 2H), 4.74 (s, 2H).

### Synthesis of 1-(4-((1H-pyrazol-1-yl)methyl)phenyl)ethan-1-ol

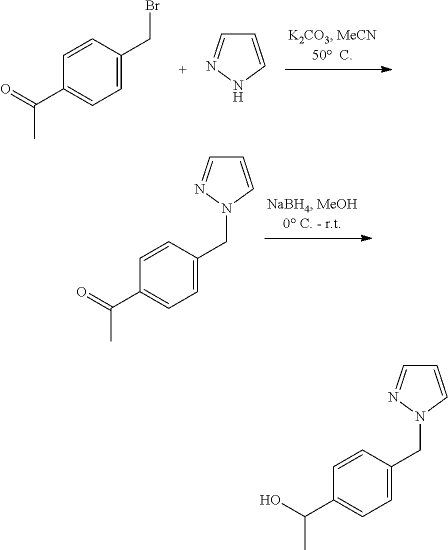

Synthesis of 1-(4-((1H-pyrazol-1-yl)methyl)phenyl)ethan-1-one. 1-(4-(bromomethyl)phenyl)ethan-1-one (1.5 g, 7.0 mmol) and K₂CO₃ (1.5 g, 7.3 mmol) were added to a stirred solution of 1H-pyrazole (0.50 g, 7.3 mmol) in MeCN (10 mL) and heated to 50° C. After 18 h the reaction was cooled, water (5.0 mL) was added and the mixture was extracted with EtOAc (3×50 mL). The combined organics were dried over MgSO₄, filtered then concentrated in vacuo to give the title compound (1.4 g, 99%), which was used in the next step without further purification. ESI-MS (M+H)+: 201.1

Synthesis of 1-(4-((1H-pyrazol-1-yl)methyl)phenyl)ethan-1-ol. NaBH₄ (0.31 g, 8.3 mmol) was added to a stirred solution of 1-(4-((1H-pyrazol-1-yl)methyl)phenyl)ethan-1-one (1.4 g, 6.9 mmol) in MeOH (30 mL) at 0° C. under a N₂ environment. After 4 h the reaction was warmed to room temperature and stirred for 18 h. Water (60 ml) was added and the mixture was extracted with EtOAc (3×60 mL). The combined organics were dried over MgSO₄, filtered then concentrated in vacuo. The residue was purified by silica gel chromatography, eluting with a gradient of 0-10% MeOH in DCM to give the title compound (1.20 g, 86%) as a yellow oil. ESI-MS (M+H)+: 203.2, ¹H NMR (400 MHz, DMSO) δ 7.81-7.80 (m, 1H), 7.46-7.45 (m, 1H), 7.32-7.28 (m, 2H), 7.19-7.16 (m, 2H), 6.27-6.26 (m, 1H), 5.30 (s, 2H), 5.13 (d, J=4.3 Hz, 1H), 4.73-4.66 (m, 1H), 1.30 (d, J=6.4 Hz, 3H).

### Synthesis of (4-((3-cyclopropyl-1H-pyrazol-1-yl)methyl)phenyl)methanol

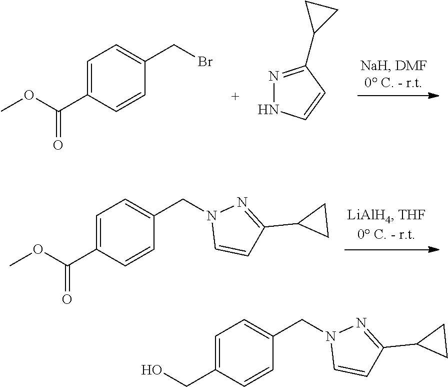 [SMILES: COC(=O)c1ccc(CBr)cc1 ; c1cc(C2CC2)n[nH]1 ; COC(=O)c1ccc(Cn2ccc(C3CC3)n2)cc1 ; OCc1ccc(Cn2ccc(C3CC3)n2)cc1]

Synthesis of methyl 4-((3-cyclopropyl-1H-pyrazol-1-yl)methyl)benzoate. A mixture of methyl 4-(bromomethyl)benzoate (4.0 g, 18 mmol) and 3-cyclopropyl-1H-pyrazole (1.5 g, 14 mmol) in DMF (40 mL) was cooled to 0° C. and NaH (60% in mineral oil, 610 mg, 15 mmol) was added portion-wise. The reaction mixture was stirred at 0° C. for 30 min. The reaction mixture was then placed under a N₂ atmosphere and the reaction was allowed to warm to room temperature and stirred for 16 h. The reaction mixture was quenched with water (50 mL) dropwise and extracted with EtOAc (3×100 mL). The combined organic layers were washed with water (3×80 mL), brine (sat. aq., 100 mL), dried over MgSO₄ and concentrated in vacuo. The residue was purified by silica gel chromatography eluting with a gradient of 0-50% EtOAc in isohexane. Additional impure material isolated was further purified by silica gel chromatography eluting with a gradient of 0-20% EtOAc in isohexane. Purified materials were combined to give the title compound (1.6 g, 46%) as a colourless oil. ESI-MS (M+H)+: 257, ¹H NMR (400 MHz, CDCl₃) δ 8.01-7.98 (m, 2H), 7.25-7.20 (m, 3H), 5.93-5.92 (m, 1H), 5.28 (s, 2H), 3.90 (s, 3H), 2.00-1.92 (m, 1H), 0.94-0.88 (m, 2H), 0.74-0.68 (m, 2H).

Synthesis of (4-((3-cyclopropyl-1H-pyrazol-1-yl)methyl)phenyl)methanol. A solution of methyl 4-((3-cyclopropyl-1H-pyrazol-1-yl)methyl)benzoate (1.6 g, 6.4 mmol) in THF (20 mL) was placed under a N₂ atmosphere and cooled to 0° C. LiAlH₄ (2.0 M in THF, 3.8 mL, 7.6 mmol) was added dropwise and the reaction mixture was allowed to warm up to room temperature and stirred for 16 h. The reaction mixture was cooled to 0° C., quenched with water (0.3 mL), aqueous sodium hydroxide solution (20%, 0.3 mL) then further water (0.9 mL). The reaction mixture was allowed to warm up to room temperature, MgSO₄ was added and the mixture was stirred for 20 min. The mixture was filtered and concentrated in vacuo to give the title compound (1.4 g, 98%) as a clear oil, which was used without further purification. ESI-MS (M+H)+: 229, ¹H NMR (400 MHz, CDCl₃) δ 7.32 (d, J=8.1 Hz, 2H), 7.20-7.16 (m, 3H), 5.89 (d, J=2.3 Hz, 1H), 5.21 (s, 2H), 4.67 (s, 2H), 1.99-1.91 (m, 1H), 0.90 (ddd, J=4.2, 6.4, 8.5 Hz, 2H), 0.72-0.67 (m, 2H).

### Synthesis of 5 (1-(4-((1H-pyrazol-1-yl)methyl)phenyl)ethoxy)-3-chloropyridazine

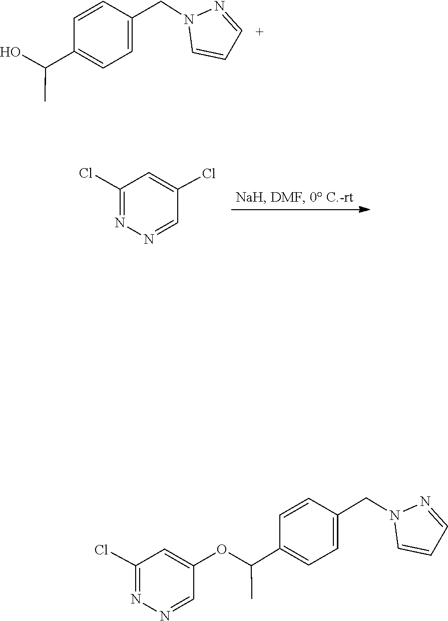 [SMILES: CC(O)c1ccc(Cn2cccn2)cc1 ; Clc1cnnc(Cl)c1 ; CC(Oc1cnnc(Cl)c1)c1ccc(Cn2cccn2)cc1]

A stirred solution of 1-(4-((1H-pyrazol-1-yl)methyl)phenyl)ethan-1-ol (1.2 g, 5.9 mmol) in DMF (10 mL) was cooled to 0° C. under a N₂ atmosphere and NaH (60% in mineral oil, 0.29 g, 7.1 mmol) was added portion-wise. The reaction mixture was stirred at 0° C. for 10 min whereupon 3,5-dichloropyridazine (0.97 g, 6.5 mmol) was added and the reaction mixture was stirred at 0° C. for further 15 min. The reaction mixture was then stirred at room temperature for 24 h. The reaction mixture was poured into a mixture of water (25 mL) and brine (25 mL) at 0° C. and extracted with EtOAc (3×50 mL). The combined organics were dried over MgSO₄ and concentrated in vacuo. The residue was purified by column chromatography on silica gel, eluting with a gradient of 20-80% EtOAc in DCM to give the title compound (1.2 g, 65%) as a cream solid. ESI-MS (M+H)+: 315.1, ¹H NMR (400 MHz, DMSO) δ 9.00 (d, J=2.5 Hz, 1H), 7.82 (d, J=1.9 Hz, 1H), 7.46-7.41 (m, 4H), 7.21 (d, J=8.3 Hz, 2H), 6.27 (dd, J=2.0, 2.0 Hz, 1H), 5.85 (q, J=6.4 Hz, 1H), 5.33 (s, 2H), 1.59 (d, J=6.4 Hz, 3H).

Using a similar procedure to that used for 5-(1-(4-((1H-pyrazol-1-yl)methyl)phenyl)ethoxy)-3-chloropyridazine, the compounds in

Table 4 were prepared from 3,5-dichloropyridazine and the indicated coupling partner.

TABLE 4

| | Coupling | |
|---|---|---|
| Compound | Partner | Data |
| 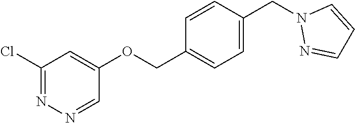 [SMILES: Clc1cc(OCc2ccc(Cn3cccn3)cc2)cnn1] | (4-((1H- pyrazol-1- yl)methyl)phe- nyl)methanol | ESI-MS (M + H)+: 301 |
| 5-((4-((1H-pyrazol-1- | | |
| yl)methyl)benzyl)oxy)-3- | | |
| chloropyridazine | | |
| 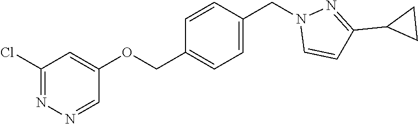 [SMILES: Clc1cc(OCc2ccc(Cn3ccc(C4CC4)n3)cc2)cnn1] | (4-((3- cyclopropyl- 1H-pyrazol-1- yl)methyl)phe- nyl)methanol | ESI-MS (M + H)+: 341, ¹H NMR (400 MHz, CDCl₃) δ 8.87 (d, J = 2.8 Hz, 1H), 7.38 - 7.34 (m, 2H), 7.25 - 7.21 (m, 3H), 6.97 (d, J = 2.5 Hz, 1H), 5.92 (d, J = 2.3 Hz, 1H), 5.26 (s, 2H), 5.13 (s, 2H), 2.05 - 1.92 (m, 1H), 0.94 - 0.89 (m, 2H), 0.73 - 0.68 (m, 2H). |
| 3-chloro-5-((4-((3-cyclopropyl-1H- | | |
| pyrazol-1- | | |
| yl)methyl)benzyl)oxy)pyridazine | | |

### 5-((4-((JH-pyrazol-1-yl methyl)benzyl)oxy)pyridazin-3-amine

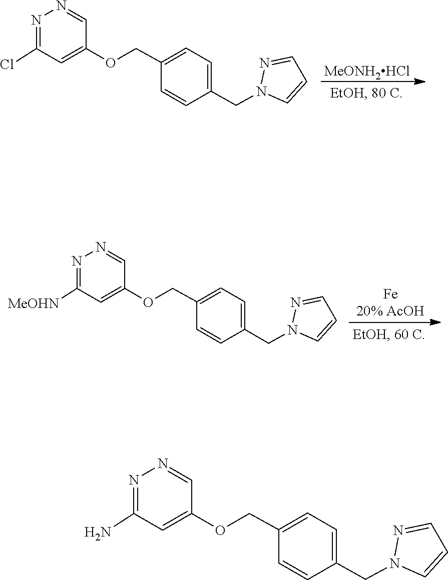

Synthesis of N-(5-((4-((1H-pyrazol-1-yl)methyl)benzyl)oxy)pyridazin-3-yl)-O— methylhydroxylamine. A mixture of 5-((4-((1H-pyrazol-1-yl)methyl)benzyl)oxy)-3-chloropyridazine (0.91 g, 3.03 mmol) and O-methyl-hydroxylamine hydrochloride (3.79 g, 45.42 mmol; 15 eq.) in ethanol (20 mL) was heated to 80° C. overnight then cooled to room temperature. The precipitated solid was removed by filtration then the filtrate was concentrated in vacuo. The residue was suspended in NaHCO₃ (sat. aq., 20 mL) then extracted with DCM (3×20 mL). The organics were passed through a phase separator cartridge then concentrated in vacuo to give the title compound which was used without further purification.

Synthesis of 5-((4-((1H-pyrazol-1-yl)methyl)benzyl)oxy)pyridazin-3-amine. N-(5-((4-((1H-pyrazol-1-yl)methyl)benzyl)oxy)pyridazin-3-yl)-O-methylhydroxylamine (0.945 g; 3.03 mmol) was dissolved in ethanol (30 mL). Acetic acid (20% v/v aqueous solution, 4.5 mL) was added followed by iron powder (0.848 g, 15.19 mmol, 5 eq.). The mixture was heated to 60° C. for 2 h, cooled to room temperature then diluted with EtOAc (40 mL). The mixture was filtered through Celite® then the filtrate concentrated in vacuo to give a beige solid. Trituration (Et₂O containing a few drops of MeOH) of the crude product which was then isolated by filtration and dried under suction gave the acetic acid salt of the title compound as a cream solid. This was partitioned between DCM and saturated aqueous NaHCO₃ solution. The organics were passed through a phase separator cartridge then concentrated in vacuo to give the title compound as a cream solid (0.543 g, 1.93 mmol, 63%). ¹H NMR (400 MHz, DMSO): δ ppm 8.26 (1H, d, J=2.5 Hz), 7.88 (1H, d, J=1.8 Hz), 7.51 (1H, d, J=1.3 Hz), 7.45 (2H, d, J=8.1 Hz), 7.29 (2H, d, J=8.1 Hz), 6.34-6.30 (2H, m), 6.27 (2H, s), 5.40 (2H, s), 5.15 (2H, s).

The compounds in Table 5 were prepared using a similar procedure to that used for 5-((4-((1H-pyrazol-1-yl)methyl)benzyl)oxy)pyridazin-3-amine.

TABLE 5

| Compound | Coupling Partner | Analytical Data |
|---|---|---|
| 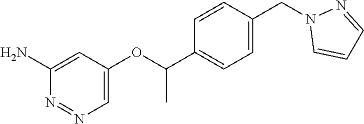 [SMILES: CC(Oc1cnnc(N)c1)c1ccc(Cn2cccn2)cc1] | N-(5-(1-(4-((1H-pyrazol-1- yl)methyl)phenyl)ethoxy)pyrid- azin-3-yl)-O- methylhydroxylamine | ESI-MS (M + H)+: 296.2. |
| 5-(1-(4-((1H-pyrazol-1- | | |
| yl)methyl)phenyl)ethoxy)pyridazin-3-amine | | |
| 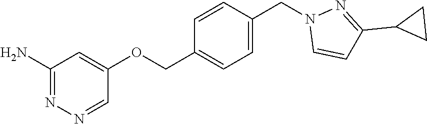 [SMILES: Nc1cc(OCc2ccc(Cn3ccc(C4CC4)n3)cc2)cnn1] | N-(5-((4-((3-cyclopropyl-1H- pyrazol-1- yl)methyl)benzyl)oxy)pyrid- azin-3-yl)-O- methylhydroxylamine | ESI-MS (M + H)+: 322.2. |
| 5-((4-((3-cyclopropyl-1H-pyrazol-1- | | |
| yl)methyl)benzyl)oxy)pyridazin-3-amine | | |

### Synthesis of (5-cyclopropylthieno[2,3-b]pyridin-2-yl)methanamine

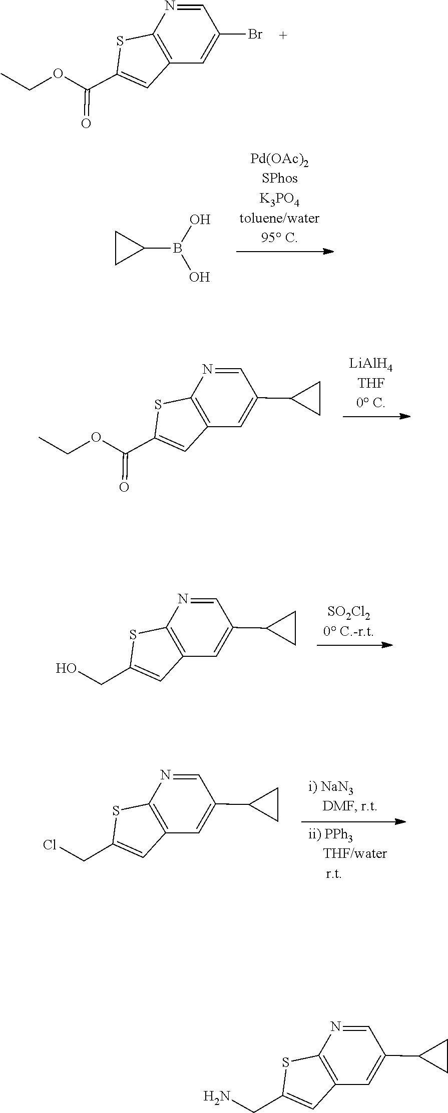 [SMILES: CCOC(=O)c1cc2cc(Br)cnc2s1 ; OB(O)C1CC1 ; CCOC(=O)c1cc2cc(C3CC3)cnc2s1 ; OCc1cc2cc(C3CC3)cnc2s1 ; ClCc1cc2cc(C3CC3)cnc2s1 ; NCc1cc2cc(C3CC3)cnc2s1]

Synthesis of ethyl 5-cyclopropylthieno[2,3-b]pyridine-2-carboxylate. A mixture of ethyl 5-bromothieno[2,3-b]pyridine-2-carboxylate (2.0 g, 7.0 mmol), cyclopropylboronic acid (1.5 g, 17 mmol), SPhos (0.29 g, 0.70 mmol) and K₃PO₄ (5.2 g, 24 mmol) in toluene (35 mL) and water (3.5 mL) was degassed with N₂. Pd(OAc)₂ (0.080 g, 0.35 mmol) was added and the mixture was stirred at 95° C. for 18 h under a N₂ atmosphere. Water (100 mL) was added and the mixture was extracted with EtOAc (3×100 mL). The combined organic layers were dried (MgSO₄), filtered and concentrated in vacuo. The residue was purified by column chromatography on silica gel, eluting with 20-100% EtOAc in cyclohexane to give the title compound (1.6 g, 91%) as a yellow oil. ESI-MS (M+H)+: 248.1, ¹H NMR (400 MHz, DMSO) δ 8.60 (d, J=2.2 Hz, 1H), 8.09 (s, 1H), 8.08 (d, J=2.2 Hz, 1H), 4.38 (q, J=7.1 Hz, 2H), 2.17-2.09 (m, 1H), 1.35 (t, J=7.0 Hz, 3H), 1.11-1.05 (m, 2H), 0.85-0.80 (m, 2H).

Synthesis of (5-cyclopropylthieno[2,3-b]pyridin-2-yl)methanol. LiAlH₄ solution (1.0 M in THF, 13 mL, 13 mmol) was added dropwise to a stirred solution of ethyl 5-cyclopropylthieno[2,3-b]pyridine-2-carboxylate (1.6 g, 6.5 mmol) in THF (32 mL) at 0° C. under a N₂ atmosphere. The mixture was stirred for 1 h at 0° C. under a N₂ atmosphere then water (0.50 mL) was added dropwise, followed by a solution of NaOH (15% aq., 0.40 mL) and further water (1.5 mL) and Et₂O (ca. 150 mL). The mixture was warmed to room temperature and stirred for 15 min. MgSO₄ was added and the mixture was stirred for a further 15 min then filtered with Et₂O. The filtrate was concentrated in vacuo to give the title compound (1.1 g, 79%). ¹H NMR (400 MHz, DMSO) δ 8.39 (d, J=2.3 Hz, 1H), 7.84 (d, J=2.0 Hz, 1H), 7.20 (s, 1H), 5.76 (t, J=5.9 Hz, 1H), 4.79 (d, J=4.8 Hz, 2H), 2.15-2.07 (m, 1H), 1.11-1.04 (m, 2H), 0.85-0.80 (m, 2H). LCMS: 206.1

Synthesis of 2-(chloromethyl)-5-cyclopropylthieno[2,3-b]pyridine. SOCl₂ (1.9 mL) was added dropwise to (5-cyclopropylthieno[2,3-b]pyridin-2-yl)methanol (1.1 g, 5.2 mmol) at 0° C. The mixture was warmed to room temperature and stirred for 1 h. The mixture was cooled to 0° C. and diluted with DCM (25 mL) then washed with water (25 mL) and NaHCO₃ (sat. aq., 25 mL). The organic layer was passed through a phase separator and concentrated in vacuo to give the title compound (0.80 g, 69%) which was used directly in the next step. ESI-MS (M+H)+: 224.1

Synthesis of (5-cyclopropylthieno[2,3-b]pyridin-2-yl)methanamine. NaN₃ (0.40 g, 6.2 mmol) was added to a solution of 2-(chloromethyl)-5-cyclopropylthieno[2,3-b]pyridine (0.80 g, 3.6 mmol) in DMF (10 mL). The mixture was stirred at room temperature for 18 h. NaHCO₃ (sat. aq., 50 mL) was added and the mixture was extracted with EtOAc (3×50 mL). The combined organic layers were dried (MgSO₄), filtered and concentrated in vacuo to give 2-(azidomethyl)-5-cyclopropylthieno[2,3-b]pyridine. The residue was dissolved in THF (5.0 mL) and PPh₃ (2.7 g, 10 mmol) and water (0.5 mL) were added. The mixture was stirred at room temperature for 18 h. The mixture was concentrated in vacuo and dissolved in HCl (1.0 M aq., 25 mL) and washed with DCM (2×25 mL). The aqueous layer was concentrated in vacuo then dissolved in MeOH (50 mL). MP-carbonate resin (12 g) was added and the mixture was stirred gently at room temperature for 1 h. The mixture was filtered and the filtrated was concentrated in vacuo to give the title compound (0.55 g, 75%) as an orange gum. ESI-MS (M+H)+: 205.1, ¹H NMR (400 MHz, DMSO) δ 8.38 (d, J=2.3 Hz, 1H), 7.83 (d, J=2.0 Hz, 1H), 7.24 (s, 1H), 4.12 (d, J=1.0 Hz, 2H), 2.15-2.07 (m, 1H), 1.11-1.05 (m, 2H), 0.85-0.80 (m, 2H).

### Synthesis of (6-cyclopropyl-8-(4-methylpiperazin-1-yl)-[1,2,4]triazolo[1,5-a]pyridin-2-yl)methanamine

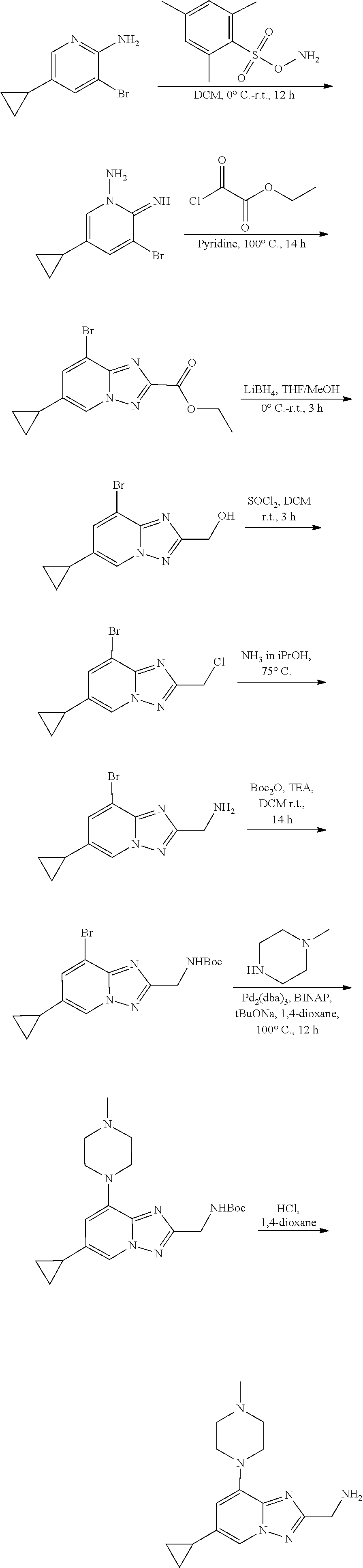

Synthesis of 3-bromo-5-cyclopropyl-2-iminopyridin-1 (2H)-amine. To a solution of 3-bromo-5-cyclopropylpyridin-2-amine (2.5 g, 12 mmol) in DCM (50 mL), 0-(mesitylsulfonyl)hydroxylamine (5.2 g, 24 mmol) was added at 0° C. The mixture was stirred at room temperature for 12 h. The reaction was concentrated in vacuo to give 3-bromo-5-cyclopropyl-2-iminopyridin-1 (2H)-amine (8 g, crude) as a yellow solid which was used in the next step directly. ESI-MS [M+H]⁺: 228.1

Synthesis of ethyl 8-bromo-6-cyclopropyl-[1,2,4]triazolo[1,5-a]pyridine-2-carboxylate. A mixture of 3-bromo-5-cyclopropyl-2-iminopyridin-1 (2H)-amine (8 g, crude) and ethyl 2-chloro-2-oxoacetate (3.3 g, 24 mmol) in pyridine (40 mL) was stirred at 100° C. for 14 h. The mixture was concentrated in vacuo and the residue was diluted with NaHCO₃ (sat. aq., 100 mL). The mixture was extracted with EtOAc (50 mL×3). The combined organic layers were washed with brine (50 mL), dried over Na₂SO₄ and concentrated in vacuo to give the crude, which was purified by column chromatography (eluent: EtOAc/PE from 0 to 50%) to give ethyl 8-bromo-6-cyclopropyl-[1,2,4]triazolo[1,5-a]pyridine-2-carboxylate (740 mg, 20%, two steps) as a yellow solid. ESI-MS [M+H]⁺: 310.1

Synthesis of (8-bromo-6-cyclopropyl-[1,2,4]triazolo[1,5-a]pyridin-2-yl)methanol. To a mixture of ethyl 8-bromo-6-cyclopropyl-[1,2,4]triazolo[1,5-a]pyridine-2-carboxylate (620 mg, 2.0 mmol)) in THF/EtOH (30 mL/5 mL) was added LiBH₄ (440 mg, 20.0 mmol) at 0° C. The mixture was stirred at room temperature for 3 h. The reaction was quenched with NH₄Cl (sat. aq., 50 mL). The mixture was extracted with EtOAc (50 mL×3). The combined organic layers were washed with brine (50 mL), dried over Na₂SO₄ then concentrated in vacuo to give the crude, which was purified by column chromatography (eluent: EtOAc/PE from 0 to 50%) to give the product (8-bromo-6-cyclopropyl-[1,2,4]triazolo[1,5-a]pyridin-2-yl)methanol (370 mg, 69%) as a white solid. ESI-MS [M+H]⁺: 268.2

Synthesis of 8-bromo-2-(chloromethyl)-6-cyclopropyl-[1,2,4]triazolo[1,5-a]pyridine. To a solution of (8-bromo-6-cyclopropyl-[1,2,4]triazolo[1,5-a]pyridin-2-yl)methanol (370 mg, 1.38 mmol)) in DCM (25 mL) was added SOCl₂ (0.5 mL). The mixture was stirred at room temperature for 2 h. The mixture was concentrated in vacuo to give the crude product 8-bromo-2-(chloromethyl)-6-cyclopropyl-[1,2,4]triazolo[1,5-a]pyridine (400 mg, crude) as a yellow solid which was used in the next step directly. ESI-MS [M+H]⁺: 286.1

Synthesis of (8-bromo-6-cyclopropyl-[1,2,4]triazolo[1,5-a]pyridin-2-yl)methanamine. A mixture of 8-bromo-2-(chloromethyl)-6-cyclopropyl-[1,2,4]triazolo[1,5-a]pyridine (400 mg, crude) in NH₃/IPA (10 mL, 2M) in a sealed tube was stirred at 75° C. for 12 h. The mixture was concentrated in vacuo to give the crude product (8-bromo-6-cyclopropyl-[1,2,4]triazolo[1,5-a]pyridin-2-yl)methanamine (500 mg, crude) as a white solid which was used in the next step directly. ESI-MS [M+H]⁺: 267.1

Synthesis of tert-butyl ((8-bromo-6-cyclopropyl-[1,2,4]triazolo[1,5-a]pyridin-2-yl)methyl)carbamate. To a solution of (8-bromo-6-cyclopropyl-[1,2,4]triazolo[1,5-a]pyridin-2-yl)methanamine (250 mg, crude) in DCM (15 mL) was added TEA (250 mg, 2.48 mmol) and (Boc)₂O (270 mg, 1.24 mmol). After stirring at room temperature for 2 h, water (50 mL) was added and extracted with DCM (3×50 mL). The organic layers were washed with brine, dried over Na₂SO₄, concentrated in vacuo and purified by silica gel chromatography (eluent: PE:EA=2:1) to give the product as a white solid (150 mg, 60% over 3 steps). ESI-MS [M+H]⁺: 367.1.

Synthesis of tert-butyl ((6-cyclopropyl-8-(4-methylpiperazin-1-yl)-[1,2,4]triazolo[1,5-a]pyridin-2-yl)methyl)carbamate. To a mixture of tert-butyl ((8-bromo-6-cyclopropyl-[1,2,4]triazolo[1,5-a]pyridin-2-yl)methyl)carbamate (220 mg, 0.6 mmol), 1-methylpiperazine (199 mg, 0.9 mmol), BINAP (75 mg, 0.12 mmol) and Pd₂(dba)₃ (110 mg, 0.12 mmol) in 1,4-dioxane (5 mL) was added t-BuONa (173 mg, 1.8 mmol). The reaction mixture was stirred at 100° C. for 12 h under N₂. The reaction was washed with water (20 mL) then extracted with EtOAc (3×30 mL). The combined organic layers were washed with brine, dried over Na₂SO₄ then concentrated in vacuo and purified by silica gel chromatography (eluent: DCM/MeOH=10:1) to give the product as a white solid (70 mg, 30%). ESI-MS [M+H]⁺: 387.1.

Synthesis of (6-cyclopropyl-8-(4-methylpiperazin-1-yl)-[1,2,4]triazolo[1,5-a]pyridin-2-yl)methanamine. A solution of tert-butyl ((6-cyclopropyl-8-(4-methylpiperazin-1-yl)-[1,2,4]triazolo[1,5-a]pyridin-2-yl)methyl)carbamate (130 mg, 0.336 mmol) and HCl (4M solution in 1,4-dioxane, 1 mL) in 1,4-dioxane (5 mL) was stirred at room temperature for 3 h. The solution was concentrated in vacuo to give the crude, which was used into the next step without purification (130 mg, crude). ESI-MS [M+H]⁺: 287.1.

### Synthesis of (6-cyclopropyl-[1,2,4]triazolo[1,5-a]pyrimidin-2-yl)methanamine

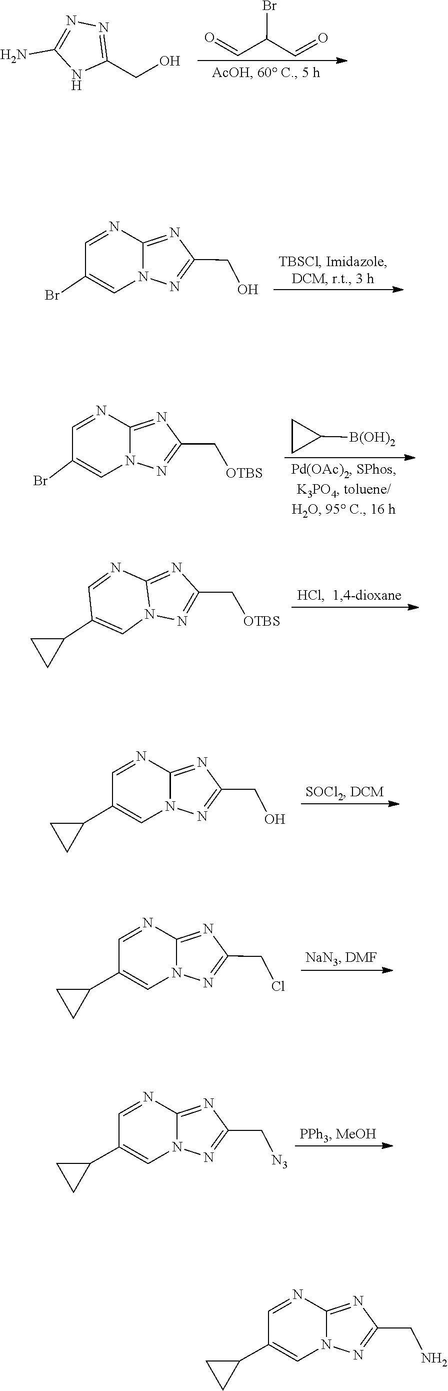

Synthesis of (6-bromo-[1,2,4]triazolo[1,5-a]pyrimidin-2-yl)methanol. To a mixture of (5-amino-4H-1,2,4-triazol-3-yl)methanol (500 mg, 4.4 mmol) in AcOH (10 mL) was added 2-bromomalonaldehyde (658 mg, 4.39 mmol). The reaction mixture was stirred at 60° C. for 5 h under N₂. The reaction was concentrated in vacuo to give the crude product, which was purified by preparative TLC (eluent: DCM:MeOH=10:1) to give (6-bromo-[1,2,4]triazolo[1,5-a]pyrimidin-2-yl)methanol (400 mg, 40%) as a white solid. ESI-MS [M+H]⁺: 229.1.

Synthesis of 6-bromo-2-(((tert-butyldimethylsilyl)oxy)methyl)-[1,2,4]triazolo[1,5-a]pyrimidine. To a mixture of (6-bromo-[1,2,4]triazolo[1,5-a]pyrimidin-2-yl)methanol (400 mg, 1.75 mmol) and imidazole (238 mg, 3.5 mmol) in DMF (10 mL) was added TBSCl (317 mg, 2.1 mmol). After stirring at room temperature for 3 h, the mixture was washed with water (20 mL), extracted with DCM (3×30 mL). The combined organic layers were washed with brine, dried over Na₂SO₄ then concentrated in vacuo to give the crude product, which was purified by Pre-TLC (eluent: EA:PE=1:2) to give 6-bromo-2-(((tert-butyldimethylsilyl)oxy)methyl)-[1,2,4]triazolo[1,5-a]pyrimidine (300 mg, 50%) as a white solid. ESI-MS [M+H]⁺: 343.1.

Synthesis of 2-(((tert-butyldimethylsilyl)oxy)methyl)-6-cyclopropyl-[1,2,4]triazolo[1,5-a]pyrimidine. To a mixture of 6-bromo-2-(((tert-butyldimethylsilyl)oxy)methyl)-[1,2,4]triazolo[1,5-a]pyrimidine (300 mg, 0.88 mmol), cyclopropylboronic acid (112 mg, 1.32 mmol) and K₃PO₄ (652 mg, 3.08 mmol) in toluene/H₂O (10 mL/2 mL) were added Pd(OAc)₂ (19.7 mg, 0.088 mmol) and S-Phos (36 mg, 0.088 mmol). The reaction mixture was stirred at 95° C. for 16 h under N₂. After cooled to room temperature, the mixture was filtered and the filtrate was concentrated in vacuo to give the crude product, which was then purified by preparative TLC (eluent: PE/EA=1/1) to give 2-(((tert-butyldimethylsilyl)oxy)methyl)-6-cyclopropyl-[1,2,4]triazolo[1,5-a]pyrimidine (200 mg, 59%) as a yellow solid. ESI-MS [M+H]⁺: 305.2.

Synthesis of (6-cyclopropyl-[1,2,4]triazolo[1,5-a]pyrimidin-2-yl)methanol. To a mixture of 2-(((tert-butyldimethylsilyl)oxy)methyl)-6-cyclopropyl-[1,2,4]triazolo[1,5-a]pyrimidine (200 mg, 0.66 mmol) in MeOH (2 mL) was added HCl (4M solution in 1,4-dioxane, 2 mL). The reaction mixture was stirred at room temperature for 2 h and then concentrated in vacuo to give (6-cyclopropyl-[1,2,4]triazolo[1,5-a]pyrimidin-2-yl)methanol (125 mg, crude) as a yellow solid. ESI-MS [M+H]⁺: 191.2.

Synthesis of 2-(chloromethyl)-6-cyclopropyl-[1,2,4]triazolo[1,5-a]pyrimidine. To a mixture of (6-cyclopropyl-[1,2,4]triazolo[1,5-a]pyrimidin-2-yl)methanol (100 mg, crude from previous step) in DCM (2 mL) was added SOCl₂ (0.5 mL). The reaction mixture was stirred at room temperature for 4 h and then concentrated in vacuo to give 2-(chloromethyl)-6-cyclopropyl-[1,2,4]triazolo[1,5-a]pyrimidine (100 mg, crude) as a yellow solid, which was used without further purification. ESI-MS [M+H]⁺: 209.1.

Synthesis of 2-(azidomethyl)-6-cyclopropyl-[1,2,4]triazolo[1,5-a]pyrimidine. A mixture of 2-(chloromethyl)-6-cyclopropyl-[1,2,4]triazolo[1,5-a]pyrimidine (100 mg, 0.48 mmol) and NaN₃ (46.8 mg, 0.72 mmol) in DMF (3 mL) was stirred at room temperature for 3 h. The reaction was then washed with water (10 mL), extracted with EA (3×20 mL). The combined organic layers were washed with brine, dried over Na₂SO₄, concentrated in vacuo to give 2-(azidomethyl)-6-cyclopropyl-[1,2,4]triazolo[1,5-a]pyrimidine (100 mg, crude) as a yellow solid, which was used into the next step without further purification. ESI-MS [M+H]⁺: 216.1.

Synthesis of (6-cyclopropyl-[1,2,4]triazolo[1,5-a]pyrimidin-2-yl)methanamine. To a mixture of 2-(azidomethyl)-6-cyclopropyl-[1,2,4]triazolo[1,5-a]pyrimidine (200 mg, 0.66 mmol) in MeOH (10 mL) was added PPh₃ (347 mg, 1.32). The resulting solution was stirred at 70° C. for 12 h. The reaction was concentrated in vacuo to give the crude, which was purified by preparative TLC (eluent: DCM/MeOH=1/8) to give (6-cyclopropyl-[1,2,4]triazolo[1,5-a]pyrimidin-2-yl)methanamine (60 mg, 68%) as a yellow solid. ESI-MS [M+H]⁺: 190.2.

### Synthesis of (6-cyclopropyl-8-(4-methylpiperazin-1-yl)imidazo[1,2-b]pyridazin-2-yl)methanamine

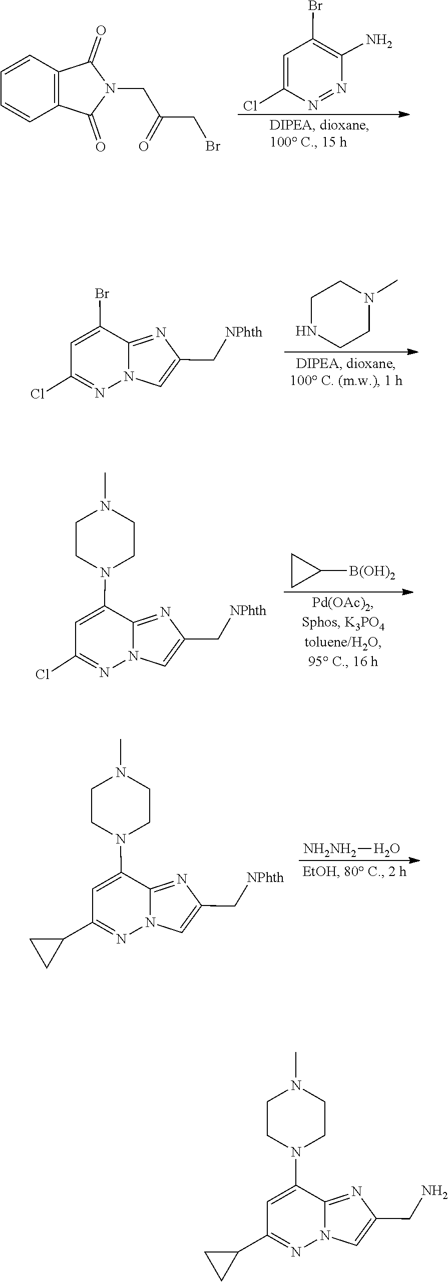

Synthesis of 2-((8-bromo-6-chloroimidazo[1,2-b]pyridazin-2-yl)methyl)isoindoline-1,3-dione. To a stirred mixture of 4-bromo-6-chloropyridazin-3-amine (737 mg, 3.6 mmol) and 2-(3-bromo-2-oxopropyl)isoindoline-1,3-dione (1 g, 3.6 mmol) in 1,4-dioxane (30 mL) was added DIPEA (1.4 g, 10.8 mmol) and the mixture was heated to 100° C. for 15 h. The reaction was quenched with NaHCO₃ (sat. aq., 50 mL) and extracted with EtOAc (50 mL×3). The combined organic layers were washed with brine (50 mL), dried over Na₂SO₄ and concentrated in vacuo to give the crude, which was purified by column chromatography (eluent: EtOAc/PE from 0 to 100%) to give 2-((8-bromo-6-chloroimidazo[1,2-b]pyridazin-2-yl)methyl)isoindoline-1,3-dione as yellow solid (700 mg, 50%). ESI-MS [M+H]⁺: 392.0.

Synthesis of 2-((6-chloro-8-(4-methylpiperazin-1-yl)imidazo[1,2-b]pyridazin-2-yl)methyl)isoindoline-1,3-dione. A mixture of 2-((8-bromo-6-chloroimidazo[1,2-b]pyridazin-2-yl)methyl)isoindoline-1,3-dione (500 mg, 1.27 mmol), 1-methylpiperazine (192 mg, 1.90 mmol) and DIPEA (500 mg, 3.84 mmol) in 1,4-dioxane (10 mL) was stirred at 100° C. for 1 h under microwave irradiation. The mixture was cooled to room temperature, diluted with water (50 mL) and extracted with EtOAc (3×30 mL). The combined organic layers were washed with brine (30 mL) and concentrated in vacuo. The residue was purified by column chromatography (eluent: MeOH/DCM=0˜ 10%) to give 2-((6-chloro-8-(4-methylpiperazin-1-yl)imidazo[1,2-b]pyridazin-2-yl)methyl)isoindoline-1,3-dione as a yellow solid (400 mg, 76%). ESI-MS [M+H]⁺: 411.1.

Synthesis of 2-((6-cyclopropyl-8-(4-methylpiperazin-1-yl)imidazo[1,2-b]pyridazin-2-yl)methyl)isoindoline-1,3-dione. To a mixture of 2-((6-chloro-8-(4-methylpiperazin-1-yl)imidazo[1,2-b]pyridazin-2-yl)methyl)-isoindoline-1,3-dione (400 mg, 0.98 mmol) in toluene /water (20 mL/2 mL) was added cyclopropylboronic acid (253 mg, 2.94 mmol), Sphos (40 mg, 0.098 mmol), Pd(OAc)₂ (22 mg, 0.098 mmol) and K₃PO₄ (623 mg, 2.94 mmol) and the mixture was stirred at 95° C. for 16 h under N₂. The mixture was quenched with water (50 mL) and extracted with EtOAc (3×50 mL). The combined organic layers were washed with brine (50 mL) and concentrated in vacuo. The residue was purified by column chromatography (eluent: MeOH/DCM=0-10%) to give 2-((6-cyclopropyl-8-(4-methylpiperazin-1-yl)imidazo[1,2-b]pyridazin-2-yl)methyl)isoindoline-1,3-dione as a yellow solid (350 mg, 86%). ESI-MS [M+H]⁺: 417.2.

Synthesis of (6-cyclopropyl-8-(4-methylpiperazin-1-yl)imidazo[1,2-b]pyridazin-2-yl)methanamine. A mixture of 2-((6-cyclopropyl-8-(4-methylpiperazin-1-yl)imidazo[1,2-b]pyridazin-2-yl)methyl)isoindoline-1,3-dione (350 mg, 0.84 mmol) and hydrazine hydrate (210 mg, 4.21 mmol) in EtOH (10 mL) was stirred at 80° C. for 3 h under N₂. The mixture was filtered and the filtrate was concentrated in vacuo to give the crude product, which was purified by column chromatography (eluent: MeOH/DCM=0-10%) to give (6-cyclopropyl-8-(4-methylpiperazin-1-yl)imidazo[1,2-b]pyridazin-2-yl)methanamine as a yellow solid (200 mg, 83%). ESI-MS [M+H]⁺: 287.3.

### Synthesis of (5-cyclopropyl-7-(4-methylpiperazin-1-yl pyrazolo[1,5-a]pyridin-2-yl)methanamine

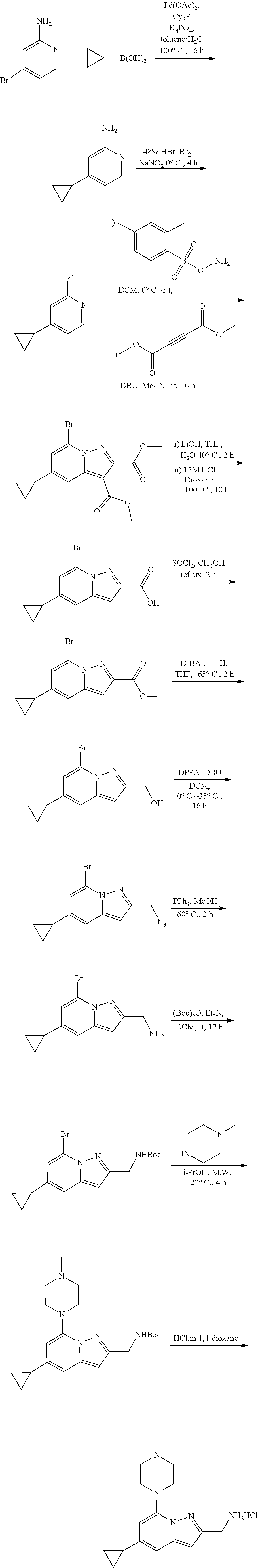

Synthesis of 4-cyclopropylpyridin-2-amine. To a solution of 4-bromopyridin-2-amine (20 g, 0.12 mol) in toluene/H₂O (200 mL/40 mL) was added cyclopropylboronic acid (20 g, 0.23 mol), Pd(OAc)₂ (1.0 g, 4.6 mmol), K₃PO₄ (74 g, 0.35 mol) and PCy₃ (2.6 g, 9.3 mmol) at room temperature. The reaction mixture was stirred at 90° C. for 16 h under nitrogen. The reaction mixture was concentrated in vacuo, diluted with water (150 mL) then extracted with EtOAc (150 ml×2). The combined organic layers were washed with brine (200 mL×1), dried over Na₂SO₄ and concentrated in vacuo give the crude product, which was purified by silica gel chromatography (eluent: EtOAc/PE=1/1) to give 4-cyclopropylpyridin-2-amine (16 g, quant.) as a yellow solid. ESI-MS [M+H]⁺: 135.1.

Synthesis of (1S,2S)-2-(2-((tert-butoxycarbonyl)amino)-6-methoxypyridin-4-yl)cyclopropane-1-carboxylic acid. To a stirred solution of HBr (40% in H₂O, 70 mL) was added 4-cyclopropylpyridin-2-amine (15 g, 0.11 mol) in portions at 0° C. and then Br₂ (52 g, 0.32 mol) was added dropwise. After stirring at 0° C. for 30 min, a solution of NaNO₂ (19 g, 0.27 mol) in water (30 mL) was added dropwise maintaining the temperature below 10° C. The resulting mixture was stirred for another 30 min at 0° C. The reaction mixture was quenched with NaOH aqueous solution (60 g in water (100 mL)) until pH=12 and extracted with EtOAc (100 mL×2). The combined organic layers were washed with water (200 mL) and brine (200 mL), dried over Na₂SO₄ and concentrated in vacuo to give the crude product, which was purified by silica gel chromatography (eluent: EtOAc/PE=1/10) to give 2-bromo-4-cyclopropylpyridine (15 g, 70%) as a yellow oil. ESI-MS [M+H]⁺: 198.1.

Synthesis of dimethyl 7-bromo-5-cyclopropylpyrazolo[1,5-a]pyridine-2,3-dicarboxylate. To a stirred solution of O-(mesitylsulfonyl)hydroxylamine (34 g, 160 mol) in DCM (200 mL) was added a solution of 2-bromo-4-cyclopropylpyridine (15 g, 76 mmol) in DCM (50 mL) slowly at 0° C. The mixture was stirred at 0° C. for 10 min then warmed to room temperature and stirred for 14 h. The reaction mixture was concentrated and dried in vacuo. The residue was dissolved in CH₃CN (200 mL) then DMAD (17 g, 120 mmol) was added followed by dropwise addition of DBU (18.2 g, 120 mol) at 0° C. The resulting mixture was stirred at 0° C. for 10 min and warmed to room temperature and stirred for 16 h. The reaction mixture was concentrated in vacuo, diluted with water (300 mL) and extracted with EtOAc (200 mL×2). The combined organic layers were washed with water (400 mL) and brine (400 mL), dried over Na₂SO₄ then concentrated in vacuo to give the crude product, which was purified by silica gel chromatography (eluent: EtOAc/PE=1/10) to give dimethyl 7-bromo-5-cyclopropylpyrazolo[1,5-a]pyridine-2,3-dicarboxylate (6.5 g, 24%) as a yellow solid. ESI-MS [M+H]⁺: 353.0.

Synthesis of 7-bromo-5-cyclopropylpyrazolo[1,5-a]pyridine-2-carboxylic acid. A mixture of dimethyl 7-bromo-5-cyclopropylpyrazolo[1,5-a]pyridine-2,3-dicarboxylate (6.0 g, 17 mmol) and LiOH—H₂O (1.4 g, 34 mmol) in THF (50 mL) and water (10 mL) was stirred at 40° C. for 2 h. The mixture was concentrated in vacuo to remove THF then 1,4-dioxane (50 mL) and HCl (conc., 12 mL) were added. The resulting mixture was stirred at 100° C. for 6 h. The reaction mixture was poured into water (200 mL) and extracted with EtOAc (150 mL×3). The combined organic layers were washed with brine (400 mL), dried over Na₂SO₄ then concentrated and dried in vacuo to give 7-bromo-5-cyclopropylpyrazolo[1,5-a]pyridine-2-carboxylic acid (4.8 g, quant.) as a yellow solid. ESI-MS [M+H]⁺: 281.0.

Synthesis of methyl 7-bromo-5-cyclopropylpyrazolo[1,5-a]pyridine-2-carboxylate. To a mixture of 7-bromo-5-cyclopropylpyrazolo[1,5-a]pyridine-2-carboxylic acid (4.8 g, 17 mmol) in MeOH (50 mL) was added dropwise of SOCl₂ (4.0 g, 34 mmol) at room temperature. The mixture was stirred at 70° C. for 3 h. The reaction mixture was concentrated in vacuo and diluted with EtOAc (200 mL), washed with NaHCO₃ (sat. aq., 150 mL) and brine (150 mL), dried over Na₂SO₄ then concentrated in vacuo to give the crude product. This was purified by silica gel chromatography (eluent: EtOAc/PE=1/10) to give methyl 7-bromo-5-cyclopropylpyrazolo[1,5-a]pyridine-2-carboxylate (3.8 g, 76% for 2 steps) as a yellow solid. ESI-MS [M+H]⁺: 295.0.

Synthesis of (7-bromo-5-cyclopropylpyrazolo[1,5-a]pyridin-2-yl)methanol. To a stirred solution of methyl 7-bromo-5-cyclopropylpyrazolo[1,5-a]pyridine-2-carboxylate (3.3 g, 11 mmol) in THF (60 mL) was added dropwise DIBAl-H (1 M in hexane, 33 mL, 33 mmol) at −65° C. The mixture was stirred at −65° C. for 2 h. The reaction mixture was quenched with NaOH (1 M in water, 50 mL) at −65° C. and warmed to room temperature, then extracted with EtOAc (100 mL×2). The combined organic layers were washed with brine (150 mL), dried over Na₂SO₄ then concentrated in vacuo to give the crude, which was purified by silica gel chromatography (eluent: EtOAc/PE=1/5) to give the desired product (2.8 g, 95%) as a yellow solid. ESI-MS [M+H]⁺: 267.0.

Synthesis of 2-(azidomethyl)-7-bromo-5-cyclopropylpyrazolo[1,5-a]pyridine. To a solution of (7-bromo-5-cyclopropylpyrazolo[1,5-a]pyridin-2-yl)methanol (2 g, 7.5 mmol) in DCM (50 mL) was added DPPA (4.1 g, 15 mmol) and DBU (2.3 g, 15 mmol) at 0° C. under N₂ and the mixture was stirred at 35° C. for 16 h. The reaction mixture was quenched with water (50 mL) and extracted with DCM (100 mL×4). The combined organic layers were washed with brine (100 mL), dried over Na₂SO₄ and concentrated in vacuo to give the crude, which was purified by column chromatography (eluent: EtOAc/PE from 0 to 10%) to give 2-(azidomethyl)-7-bromo-5-cyclopropylpyrazolo[1,5-a]pyridine (2 g, 91%) as colorless oil. ESI-MS [M+H]⁺: 292.0.

Synthesis of (7-bromo-5-cyclopropylpyrazolo[1,5-a]pyridin-2-yl)methanamine. To a solution of 2-(azidomethyl)-7-bromo-5-cyclopropylpyrazolo[1,5-a]pyridine (2 g, 6.85 mmol) in MeOH (40 mL) was added PPh₃ (2.25 g, 8.59 mmol) at 0° C. under N₂ and the mixture was stirred at 60° C. for 2 h. The reaction mixture was concentrated in vacuo to give the crude which was purified by column chromatography (eluent: MeOH/DCM from 0 to 10%) to give (7-bromo-5-cyclopropylpyrazolo[1,5-a]pyridin-2-yl)methanamine (1.2 g, 66%) as colorless oil. ESI-MS [M+H]⁺: 266.1.

Synthesis of tert-butyl ((7-bromo-5-cyclopropylpyrazolo[1,5-a]pyridin-2-yl)methyl)carbamate. A mixture of (7-bromo-5-cyclopropylpyrazolo[1,5-a]pyridin-2-yl)methanamine (1.2 g, 4.5 mmol), (Boc)₂O (1.97 g, 9.1 mmol) and Et₃N (1.37 g, 13.6 mmol) in DCM (25 mL) was stirred at room temperature for 16 h. The mixture was quenched with water (50 mL) and extracted with EtOAc (30 mL×3). The combined organic layers were washed with brine (30 mL), dried over Na₂SO₄ and concentrated in vacuo. The residue was purified by flash column chromatography (eluent: EtOAc/PE from 0 to 25%) to give tert-butyl ((7-bromo-5-cyclopropylpyrazolo[1,5-a]pyridin-2-yl)methyl)carbamate (1.17 g, 71%) as a white solid. ESI-MS [M+H]⁺: 366.1.

Synthesis of 2-bromo-N-((5-cyclopropyl-7-(4-methylpiperazin-1-yl)pyrazolo[1,5-a]pyridin-2-yl)methyl)pyridin-4-amine. A mixture of tert-butyl ((7-bromo-5-cyclopropylpyrazolo[1,5-a]pyridin-2-yl)methyl)carbamate (300 mg, 0.82 mmol), 1-methylpiperazine (164 mg, 1.64 mmol) and DIPEA (704 mg, 5.46 mmol) in i-PrOH (6 mL) was stirred in a sealed tube. After degassing with N₂ for 1 min, the reaction was irradiated in a microwave reactor at 120° C. for 4 h. The reaction was concentrated in vacuo to give the crude product, which was purified by preparative TLC (eluent: DCM/MeOH=50/1) to give tert-butyl ((5-cyclopropyl-7-(4-methylpiperazin-1-yl)pyrazolo[1,5-a]pyridin-2-yl)methyl)carbamate (260 mg, 82%) as a yellow solid. ESI-MS [M+H]⁺: 386.2.

Synthesis of (5-cyclopropyl-7-(4-methylpiperazin-1-yl)pyrazolo[1,5-a]pyridin-2-yl)methanamine. A mixture of tert-butyl ((5-cyclopropyl-7-(4-methylpiperazin-1-yl)pyrazolo[1,5-a]pyridin-2-yl)methyl)carbamate (260 mg, 0.68 mmol) in HCl (4M solution in 1,4-dioxane, 5 mL) was stirred at room temperature for 1 h. The reaction was concentrated in vacuo to give (5-cyclopropyl-7-(4-methylpiperazin-1-yl)pyrazolo[1,5-a]pyridin-2-yl)methanamine as the hydrochloric acid salt (260 mg, quant) as a yellow solid. which was used in the next step without further purification. ESI-MS [M+H]⁺: 286.2.

### Synthesis of 1-(2-(aminomethyl)-5-cyclopropylpyrazolo[1,5-a]pyridin-7-yl)pyrrolidin-2-one

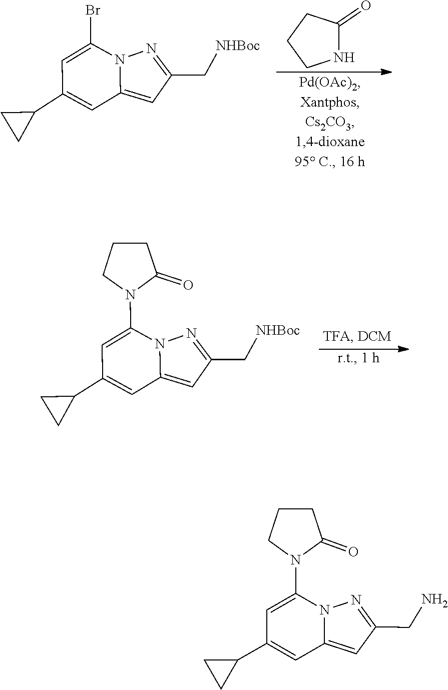 [SMILES: CC(C)(C)OC(=O)NCc1cc2cc(C3CC3)cc(Br)n2n1 ; O=C1CCCN1 ; CC(C)(C)OC(=O)NCc1cc2cc(C3CC3)cc(N3CCCC3=O)n2n1 ; NCc1cc2cc(C3CC3)cc(N3CCCC3=O)n2n1]

Synthesis of tert-butyl ((5-cyclopropyl-7-(2-oxopyrrolidin-1-yl)pyrazolo[1,5-a]pyridin-2-yl)methyl)carbamate. A mixture of tert-butyl ((7-bromo-5-cyclopropylpyrazolo[1,5-a]pyridin-2-yl)methyl)carbamate (90 mg, 0.25 mmol), pyrrolidin-2-one (42 mg, 0.49 mmol), Pd(OAc)₂ (11.2 mg, 0.05 mmol), Xantphos (29 mg, 0.05 mmol) and Cs₂CO₃ (204 mg, 0.63 mmol) in 1,4-dioxane (4 mL) was stirred at 95° C. for 16 h under nitrogen. The reaction mixture was cooled to room temperature, then filtered through Celite® and the filter cake was washed with DCM/MeOH (10/1, 10 mL). The filtrate was concentrated in vacuo to give the crude, which was purified by preparative HPLC (eluent: PE/EA=1/1) to give tert-butyl ((5-cyclopropyl-7-(2-oxopyrrolidin-1-yl)pyrazolo[1,5-a]pyridin-2-yl)methyl)carbamate (75 mg, 82%) as yellow solid. ESI-MS [M+H]⁺: 371.2.

Synthesis of 1-(2-(aminomethyl)-5-cyclopropylpyrazolo[1,5-a]pyridin-7-yl)pyrrolidin-2-one. To a solution of tert-butyl ((5-cyclopropyl-7-(2-oxopyrrolidin-1-yl)pyrazolo[1,5-a]pyridin-2-yl)methyl)carbamate (75 mg, 0.2 mmol) in DCM (2 mL) was added TFA (0.4 mL) at room temperature and the mixture was stirred at room temperature for 1 h. The reaction mixture was concentrated in vacuo and washed with saturated aq. NaHCO₃ (30 mL). The organic layer was dried and concentrated in vacuo to give 1-(2-(aminomethyl)-5-cyclopropylpyrazolo[1,5-a]pyridin-7-yl)pyrrolidin-2-one (40 mg, 73%) as yellow solid which was used without further purification. ESI-MS [M+H]⁺: 271.2.

### Synthesis of (6-cyclopropylquinolin-3-yl)methanamine hydrochloride

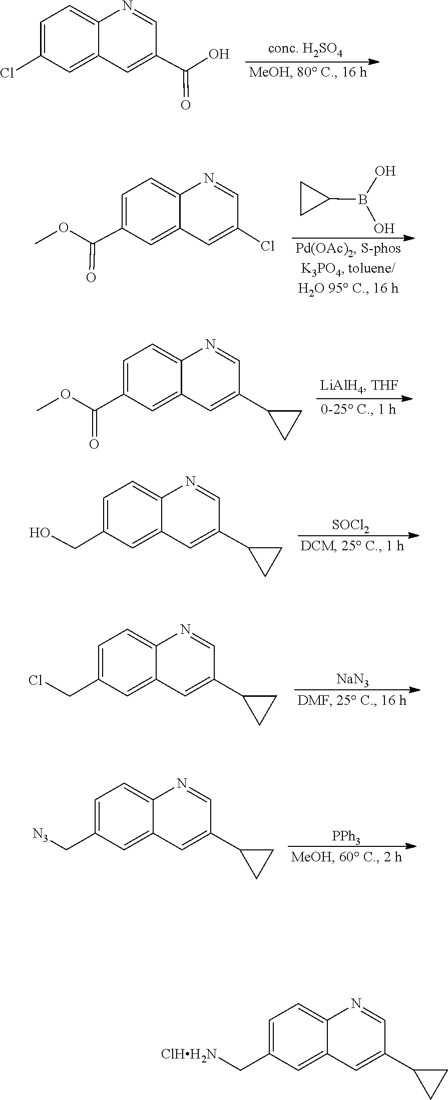

Synthesis of methyl 6-chloroquinoline-3-carboxylate. To a mixture of 6-chloroquinoline-3-carboxylic acid (1 g, 4.5 mmol) in MeOH (10 mL) was added concentrated H₂SO₄ (0.1 mL) slowly at 0° C. under N₂. The reaction mixture was then stirred at 80° C. for 16 h, cooled to room temperature and then concentrated in vacuo to give methyl 3-chloroquinoline-6-carboxylate (800 mg, crude) as a yellow solid. ESI-MS [M+H]⁺: 222.1

Synthesis of methyl 6-cyclopropylquinoline-3-carboxylate. A mixture of methyl 6-chloroquinoline-3-carboxylate (700 mg, 3.15 mmol), cyclopropylboronic acid (407 mg, 4.73 mmol), Pd(OAc)₂ (70 mg, 0.3 mmol), S-phos (139 mg, 0.3 mmol) and K₃PO₄ (2 g, 9.47) in a solution of toluene (30 mL) and H₂O (3 mL) was stirred at 95° C. for 16 h under N₂. After cooling to 25° C., water (50 mL) was added and the mixture was extracted with EtOAc (3×30 mL). The combined organic layers were washed with brine (60 mL), dried over anhydrous Na₂SO₄, and concentrated in vacuo to give the crude, which was purified by column chromatography (eluent: MeOH/DCM=1/10) to give methyl 3-cyclopropylquinoline-6-carboxylate (750 mg, crude) as a yellow solid. ESI-MS [M+H]⁺: 228.1

Synthesis of (3-cyclopropylquinolin-6-yl)methanol. To a mixture of methyl 6-cyclopropylquinoline-3-carboxylate (750 mg, 3.3 mmol) in THF (10 mL) was added LiAlH₄ (1 M in THF, 6.6 mL) slowly over several minutes at 0° C. under N₂. The reaction mixture was then slowly raised to 25° C. and stirred for 1 h. The reaction was quenched with water (20 mL) and filtered. The filtrate was concentrated in vacuo to give the crude product, which was purified by column chromatography (eluent: MeOH/DCM=1/10) to give (3-cyclopropylquinolin-6-yl)methanol (440 mg, 67% yield) as a yellow oil. ESI-MS [M+H]⁺: 200.2

Synthesis of 3-(chloromethyl)-6-cyclopropylquinoline. To a mixture of (3-cyclopropylquinolin-6-yl)methanol (440 mg, 2.2 mmol) in DCM (15 mL ) was added SOCl₂ (3.27 g, 27.5 mmol) slowly. The reaction mixture was then stirred at 25° C. for 1 h. The reaction was concentrated in vacuo to give 6-(chloromethyl)-3-cyclopropylquinoline (500 mg, crude) as a yellow solid. ESI-MS [M+H]⁺: 218.1

Synthesis of 3-(azidomethyl)-6-cyclopropylquinoline. To a mixture of 3-(chloromethyl)-6-cyclopropylquinoline (500 mg, 2.29 mmol) in DMF (10 mL) was added NaN₃ (813 mg, 6.89 mmol) slowly over several minutes at 0° C. The reaction mixture was then slowly raised to 25° C. and stirred for 16 h. The reaction was diluted with water (150 mL), extracted with EtOAc (3×20 mL). The combined organic layers were washed with brine (30 mL), dried over Na₂SO₄ and concentrated in vacuo to give 6-(azidomethyl)-3-cyclopropylquinoline (500 mg, crude). ESI-MS [M+H]⁺: 225.1

Synthesis of (6-cyclopropylquinolin-3-yl)methanamine hydrochloride. To a mixture of 3-(azidomethyl)-6-cyclopropylquinoline (500 mg, 2.22 mmol) in MeOH (15 mL) was added PPh₃ (877 mg, 3.34 mmol). The reaction mixture was stirred at 60° C. for 2 h and cooled to room temperature. The mixture was concentrated in vacuo, diluted with EtOAc (20 mL) and treated with aq. HCl (1 M, 20 mL). The aqueous phase was concentrated in vacuo to give (6-cyclopropylquinolin-3-yl)methanamine hydrochloride as a yellow solid (670 mg, crude). ESI-MS [M+H]⁺: 199.2.

### Synthesis of (3-cyclopropylquinolin-6-yl)methanamine hydrochloride

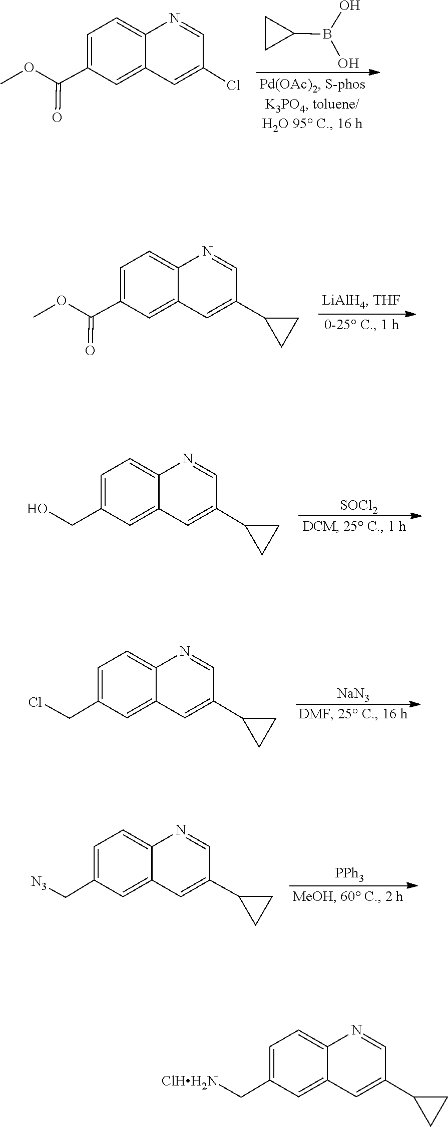

Synthesis of methyl 3-cyclopropylquinoline-6-carboxylate. A mixture of methyl 3-chloroquinoline-6-carboxylate (500 mg, 2.25 mmol), cyclopropylboronic acid (290 mg, 3.38 mmol), Pd(OAc)₂ (50 mg, 0.22 mmol), S-phos (99.5 mg, 0.225 mmol) and K₃PO₄ in a solution of toluene (20 mL) and water (2 mL) was stirred at 95° C. for 16 h under N₂. After cooled to room temperature, water (50 mL) was added and the mixture was extracted with EtOAc (3×30 mL). The combined organic layers were washed with brine (60 mL), dried over anhydrous Na₂SO₄, and concentrated in vacuo to give the crude, which was purified by column chromatography (eluent: MeOH/DCM=1/10) to give methyl 3-cyclopropylquinoline-6-carboxylate (610 mg, crude) as a yellow solid. ESI-MS [M+H]⁺: 228.1

Synthesis of (3-cyclopropylquinolin-6-yl)methanol. To a mixture of methyl 3-cyclopropylquinoline-6-carboxylate (610 mg, 2.68 mmol) in THF (10 mL) was added LiAlH₄ (1 M in THF, 5.4 mL) slowly over several minutes at 0° C. under N₂. The reaction mixture was then slowly raised to 25° C. and stirred for 1 h. The reaction was quenched with water (20 mL) and filtered. The filtrate was concentrated in vacuo to give the crude product, which was purified by column chromatography (eluent: MeOH/DCM=1/10) to give (3-cyclopropylquinolin-6-yl)methanol (520 mg, yield: 97%) as a brown solid. ESI-MS [M+H]⁺: 200.2

Synthesis of 6-(chloromethyl)-3-cyclopropylquinoline. To a mixture of (3-cyclopropylquinolin-6-yl)methanol (520 mg, 2.6 mmol) in DCM (15 mL ) was added SOCl₂ (3.27 g, 27.5 mmol) slowly. The reaction mixture was then stirred at 25° C. for 1 h. The reaction was concentrated in vacuo to give 6-(chloromethyl)-3-cyclopropylquinoline (600 mg, crude) as a yellow solid. ESI-MS [M+H]⁺: 218.1

Synthesis of 6-(azidomethyl)-3-cyclopropylquinoline. To a mixture of 6-(chloromethyl)-3-cyclopropylquinoline (600 mg, 2.75 mmol) in DMF (10 mL) was added NaN₃ (426 mg, 2.14 mmol) slowly over several minutes at 0° C. under N₂. The reaction mixture was then slowly raised to 25° C. and stirred for 16 h. The reaction was diluted with water (150 mL), extracted with EtOAc (3×20 mL). The organic layers was washed with brine (30 mL), dried over Na₂SO₄, concentrated in vacuo to give the crude product, which was purified by column chromatography (eluent: MeOH/DCM=1/10) to give 6-(azidomethyl)-3-cyclopropylquinoline (320 mg, 51.8%) as a yellow oil. ESI-MS [M+H]⁺: 225.1

Synthesis of (3-cyclopropylquinolin-6-yl)methanamine hydrochloride. To a mixture of 6-(azidomethyl)-3-cyclopropylquinoline (320 mg, 1.42 mmol) in MeOH (15 mL) was added PPh₃ (561 mg, 2.14 mmol). The reaction mixture was stirred at 60° C. for 2 h and then cooled to room temperature. The mixture was concentrated in vacuo, diluted with EtOAc (20 mL) and treated with aq. HCl (1 M, 20 mL). The aqueous phase was concentrated in vacuo to give (3-cyclopropylquinolin-6-yl)methanamine hydrochloride (300 mg, yield: 90%). ESI-MS [M+H]⁺: 199.2

Synthesis of (6-cyclopropyl-[1,2,4]triazolo[1,5-a]pyridin-2-yl)methanamine

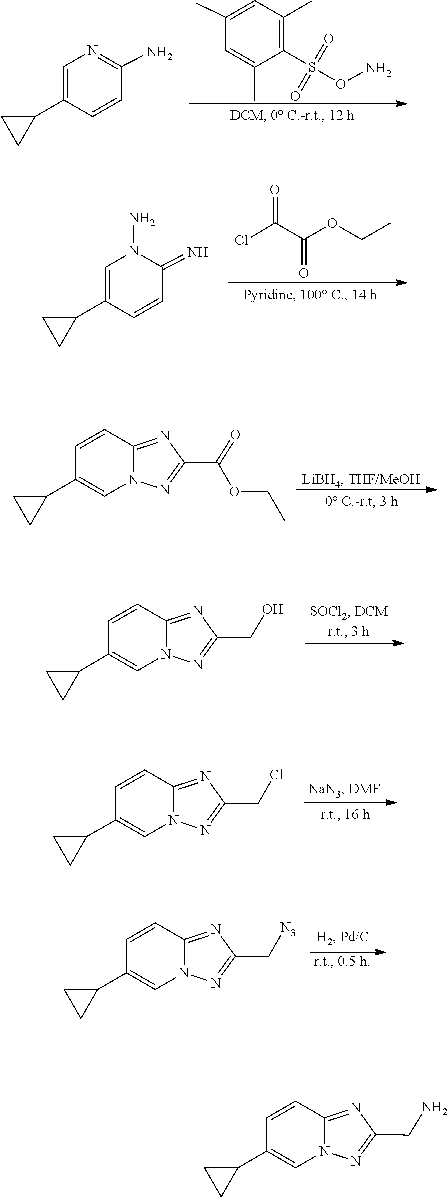

Synthesis of 5-cyclopropyl-2-iminopyridin-1 (2H)-amine. To a mixture of 5-cyclopropylpyridin-2-amine (2.0 g, 14.9 mmol) in DCM (50 mL) was added 0-(mesitylsulfonyl)hydroxylamine (6.4 g, 29.9 mmol) at 0° C. The mixture was stirred at room temperature for 12 h. The reaction was concentrated in vacuo to give 5-cyclopropyl-2-iminopyridin-1 (2H)-amine (9 g, crude) as a yellow solid, which was used in the next step directly. ESI-MS [M+H]⁺: 150.1

Synthesis of ethyl 6-cyclopropyl-[1,2,4]triazolo[1,5-a]pyridine-2-carboxylate. A mixture of 3-bromo-5-cyclopropyl-2-iminopyridin-1 (2H)-amine (9 g, crude) and ethyl 2-chloro-2-oxoacetate (4.1 g, 30 mmol) in pyridine (40 mL) was stirred at 100° C. for 14 h. The mixture was concentrated in vacuo and the residue was diluted with NaHCO₃ (sat. aq., 100 mL). The mixture was extracted with EtOAc (50 mL×3). The combined organic layers were washed with brine (50 mL), dried over Na₂SO₄, concentrated in vacuo to give the crude, which was purified by column chromatography (eluent: EtOAc/PE from 0 to 50%) to give ethyl 6-cyclopropyl-[1,2,4]triazolo[1,5-a]pyridine-2-carboxylate (1.0 g, 29% for two steps) as a yellow solid. ESI-MS [M+H]⁺: 232.1

Synthesis of (6-cyclopropyl-[1,2,4]triazolo[1,5-a]pyridin-2-yl)methanol. To a solution of ethyl 8-bromo-6-cyclopropyl-[1,2,4]triazolo[1,5-a]pyridine-2-carboxylate (800 mg, 3.46 mmol)) in THF/EtOH (30 mL/5 mL) was added LiBH₄ (1.5 g, 20.0 mmol) at 0° C. The mixture was stirred at room temperature for 3 h and then quenched with NH₄Cl (sat. aq., 50 mL). The mixture was extracted with EtOAc (50 mL×3). The combined organic layers were washed with brine (50 mL), dried over Na₂SO₄, concentrated in vacuo to give the crude, which was purified by column chromatography (eluent: EtOAc/PE from 0 to 50%) to give (6-cyclopropyl-[1,2,4]triazolo[1,5-a]pyridin-2-yl)methanol (450 mg, 69%) as a white solid. ESI-MS [M+H]⁺: 190.1.

Synthesis of 2-(chloromethyl)-6-cyclopropyl-[1,2,4]triazolo[1,5-a]pyridine. A mixture of (6-cyclopropyl-[1,2,4]triazolo[1,5-a]pyridin-2-yl)methanol (450 mg, 2.37 mmol) in DCM (25 mL) and SOCl₂ (1.0 mL) was stirred at room temperature for 2 h. The mixture was concentrated in vacuo give 2-(chloromethyl)-6-cyclopropyl-[1,2,4]triazolo[1,5-a]pyridine (500 mg, crude) as a yellow solid which was used in the next step directly. ESI-MS [M+H]⁺: 208.1

Synthesis of 2-(azidomethyl)-6-cyclopropyl-[1,2,4]triazolo[1,5-a]pyridine. To a solution of 2-(chloromethyl)-6-cyclopropyl-[1,2,4]triazolo[1,5-a]pyridine (500 mg, crude) in DMF (10 mL) was added NaN₃ (248 mg, 3.81 mmol) and the mixture was stirred at room temperature for 16 h. The reaction mixture was poured into water (50 mL) and extracted with EtOAc (2×50 ml). The combined organic layers were washed with brine (3×100 mL), dried over Na₂SO₄, concentrated and dried in vacuo to give 2-(azidomethyl)-6-cyclopropyl-[1,2,4]triazolo[1,5-a]pyridine (500 mg, crude) as a yellow solid. ESI-MS [M+H]⁺: 215.1.

Synthesis of (6-cyclopropyl-[1,2,4]triazolo[1,5-a]pyridin-2-yl)methanamine. A mixture of 2-(azidomethyl)-6-cyclopropyl-[1,2,4]triazolo[1,5-a]pyridine (500 mg, 2.33 mmol) and Pd/C (120 mg) in MeOH (30 mL) was stirred at room temperature for 30 min under an atmosphere of H₂. The reaction mixture was filtered and the filtrate was concentrated, dried in vacuo to give (6-cyclopropyl-[1,2,4]triazolo[1,5-a]pyridin-2-yl)methanamine (350 mg, 78%) as a colorless syrup. ESI-MS [M+H]⁺: 189.2.

### Synthesis of 2-(azidomethyl)-6-cyclopropyl-[1,2,4]triazolo[1,5-b]pyridazine

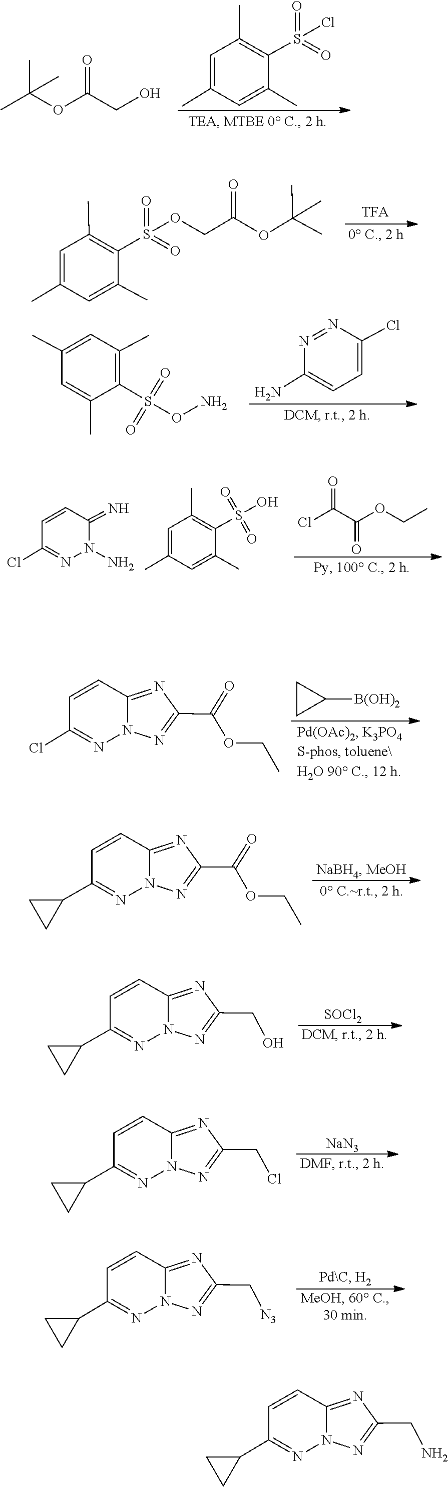

Synthesis of tert-butyl ((mesitylsulfonyl)oxy)carbamate. To a solution of tert-butyl hydroxycarbamate (20 g, 91.2 mmol) in MTBE (400 mL) was added 2,4,6-trimethylbenzenesulfonyl chloride (12 g, 91.2 mmol) and TEA (9.7 g, 96 mmol) at 0° C. The mixture was stirred at 0° C. for 2 h. The reaction mixture was filtered and the filtrate was concentrated in vacuo to give tert-butyl ((mesitylsulfonyl)oxy)carbamate (28 g, crude) as a yellow solid. ESI-MS [M+H]⁺: 316.1

Synthesis of O-(mesitylsulfonyl)hydroxylamine. A solution of tert-butyl ((mesitylsulfonyl)oxy)carbamate (28 g, 88.9 mmol) in TFA (150 mL) was stirred at 0° C. for 2 h, The mixture was poured into ice-water and stirred for 10 min to give a precipitate which was collected by filtration then dried in vacuo to give O-(mesitylsulfonyl)hydroxylamine (6.8 g, 36%) as a white solid. ESI-MS [M+H]⁺: 216.1

Synthesis of 3-chloro-6-iminopyridazin-1 (6H)-amine 2,4,6-trimethylbenzenesulfonate. To a solution of 6-chloropyridazin-3-amine (450 mg, 3.47 mmol) in DCM (20 mL) was added 0-(mesitylsulfonyl)hydroxylamine (748 mg, 3.47 mmol). The mixture was stirred at room temperature for 2 h. The resulting precipitate was collected and dried in vacuo to give 3-chloro-6-iminopyridazin-1 (6H)-amine 2,4,6-trimethylbenzenesulfonate (1.2 g, crude) as a white solid. ESI-MS [M+H]⁺: 145.1.

Synthesis of ethyl 6-chloro-[1,2,4]triazolo[1,5-b]pyridazine-2-carboxylate. To a solution of 3-chloro-6-iminopyridazin-1 (6H)-amine 2,4,6-trimethylbenzenesulfonate (100 mg, 0.33 mmol) in pyridine (2 mL) was added ethyl 2-chloro-2-oxoacetate (89 mg, 0.66 mmol). The mixture was stirred at 100° C. for 2 h. After cooling to room temperature, the reaction mixture was concentrated in vacuo to give the crude, which was purified by column chromatography (eluent: DCM/MeOH=1/50) to give ethyl 6-chloro-[1,2,4]triazolo[1,5-b]pyridazine-2-carboxylate (40 mg, 53%) as a white solid. ESI-MS [M+H]⁺: 227.1.

Synthesis of ethyl 6-cyclopropyl-[1,2,4]triazolo[1,5-b]pyridazine-2-carboxylate. To a mixture of ethyl 6-chloro-[1,2,4]triazolo[1,5-b]pyridazine-2-carboxylate (522 mg, 2.3 mmol), K₃PO₄ (1.5 g, 6.9 mmol), H₂O (1.2 mL) in toluene (12 mL) was added Pd(OAc)₂ (52 mg, 0.23 mmol) and S-phos (94 mg, 0.23 mmol). The mixture was stirred at 95° C. for 12 h under N₂ and cooled to room temperature. The reaction mixture was filtered through Celite® and the filter cake was washed with DCM/MeOH (10/1, 100 mL). The filtrate was concentrated in vacuo to give the crude, which was purified by column chromatography (DCM/MeOH=30/1) to give ethyl 6-cyclopropyl-[1,2,4]triazolo[1,5-b]pyridazine-2-carboxylate (300 mg, 56%) as a gray solid. ESI-MS [M+H]⁺: 233.1.

Synthesis of ethyl 6-cyclopropyl-[1,2,4]triazolo[1,5-b]pyridazine-2-carboxylate. To a solution of ethyl 6-cyclopropyl-[1,2,4]triazolo[1,5-b]pyridazine-2-carboxylate (280 mg, 1.2 mmol) in MeOH (10 mL) was added NaBH₄ (137 mg, 3.6 mmol) slowly at 0° C. The mixture was stirred at room temperature for 2 h. H₂O (50 mL) was added and the mixture was extracted with EtOAc (3×30 mL). The combined organic layers were washed with brine (60 mL), dried over anhydrous Na₂SO₄, and concentrated in vacuo to give (6-cyclopropyl-[1,2,4]triazolo[1,5-b]pyridazin-2-yl)methanol (120 mg, 53%) as a gray solid. ESI-MS [M+H]⁺: 191.1.

Synthesis of (6-cyclopropyl-[1,2,4]triazolo[1,5-b]pyridazin-2-yl)methanol. To a solution of (6-cyclopropyl-[1,2,4]triazolo[1,5-b]pyridazin-2-yl)methanol (200 mg, 1.06 mmol) in DCM (8 mL) was added SOCl₂ (375.6 mg, 3.16 mmol) slowly at 0° C. The mixture was stirred at room temperature for 2 h. The reaction mixture was concentrated in vacuo to give 2-(chloromethyl)-6-cyclopropyl-[1,2,4]triazolo[1,5-b]pyridazine (220 mg, crude) as a yellow solid. ESI-MS [M+H]⁺: 209.1.

Synthesis of 2-(azidomethyl)-6-cyclopropyl-[1,2,4]triazolo[1,5-b]pyridazine. To a solution of 2-(chloromethyl)-6-cyclopropyl-[1,2,4]triazolo[1,5-b]pyridazine (220 mg, 1.06 mmol) in DMF (30 mL) was added NaN₃ (103 mg, 1.59 mmol). The mixture was stirred at room temperature for 2 h. Water (50 mL) was added and the mixture was extracted with EtOAc (3×30 mL). The combined organic layers were washed with brine (60 mL), dried over anhydrous Na₂SO₄, and concentrated in vacuo to give the crude, which was purified by column chromatography (DCM/MeOH=60/1) to give 2-(azidomethyl)-6-cyclopropyl-[1,2,4]triazolo[1,5-b]pyridazine (182 mg, 80%) as a gray solid. ESI-MS [M+H]⁺: 216.1.

Synthesis of 2-(azidomethyl)-6-cyclopropyl-[1,2,4]triazolo[1,5-b]pyridazine. To a solution of 2-(azidomethyl)-6-cyclopropyl-[1,2,4]triazolo[1,5-b]pyridazine (180 mg, 0.84 mmol) in MeOH (5 mL) was added Pd\C (40 mg). The mixture was stirred at 60° C. for 30 min under H₂. The reaction mixture was filtered and the filtrate was concentrated in vacuo to give (6-cyclopropyl-[1,2,4]triazolo[1,5-b]pyridazin-2-yl)methanamine (160 mg, crude) as a gray solid. ESI-MS [M+H]⁺: 190.2.

### Synthesis of (6-cyclopropylimidazo[1,2-a]pyrimidin-2-yl)methanamine

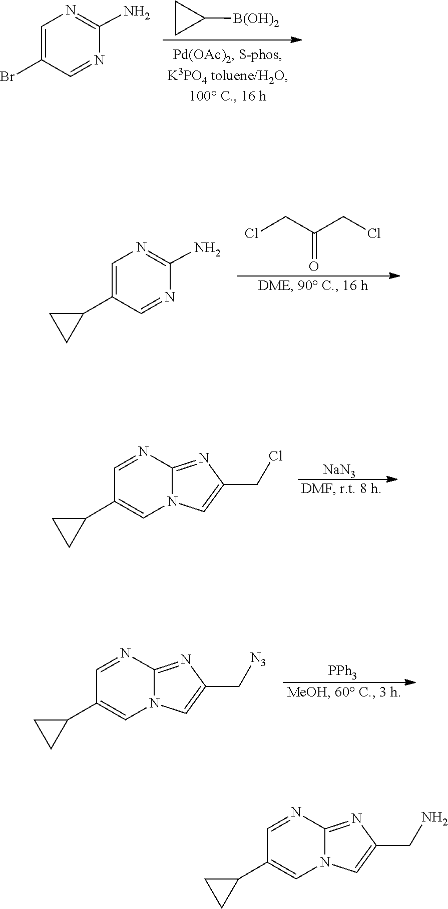

Synthesis of 5-cyclopropylpyrimidin-2-amine. A mixture of 5-bromopyrimidin-2-amine (5 g, 28.7 mmol), cyclopropylboronic acid (7.4 g, 86.2 mmol), Pd(OAc)₂ (643.5 mg, 2.87 mmol), S-Phos (1.18 g, 2.87 mmol), K₃PO₄ (18.3 g, 86.2 mmol) in toluene/water (100 mL/10 mL) was stirred at 100° C. for 16 h under N₂. The reaction mixture was filtered through Celite® and the filter cake was washed with DCM/MeOH (10/1, 100 mL). The filtrate was concentrated in vacuo to give the crude, which was purified by column chromatography (eluent: EtOAc/PE from 0 to 50%) to give 5-cyclopropylpyrimidin-2-amine (2.8 g, 72%) as a yellow solid. ESI-MS [M+H]⁺: 136.0.

Synthesis of 2-(chloromethyl)-6-cyclopropylimidazo[1,2-a]pyrimidine. A mixture of 5-cyclopropylpyrimidin-2-amine (2 g, 14.8 mmol), 1,3-dichloropropan-2-one (5.63 g, 44.4 mmol) in DME (20 mL) was stirred at 90° C. for 16 h. The reaction mixture was poured into water (50 mL) and extracted with DCM (60 mL×3). The combined organic layers were washed with brine (60 mL), dried over Na₂SO₄ and concentrated in vacuo. The residue was purified by column chromatography (eluent: DCM:MeOH from 0 to 10%) to give 2-(chloromethyl)-6-cyclopropylimidazo[1,2-a]pyrimidine (1.4 g, yield 46%) as a yellow solid. ESI-MS [M+H]⁺: 208.0

Synthesis of 2-(azidomethyl)-6-cyclopropylimidazo[1,2-a]pyrimidine. To a mixture of 2-(chloromethyl)-6-cyclopropylimidazo[1,2-a]pyrimidine (100 mg, 0.48 mmol) in DMF (10 mL) was added NaN₃ (47 mg, 0.72 mmol) at room temperature. The mixture was stirred at room temperature for 8 h. Water (50 mL) was added and the mixture was extracted with EtOAc (3×30 mL). The combined organic layers were washed with brine, dried over Na₂SO₄, concentrated in vacuo to give the crude, which was purified by preparative TLC to give the 2-(azidomethyl)-6-cyclopropylimidazo[1,2-a]pyrimidine (100 mg, 97%) as a yellow solid. ESI-MS [M+H]⁺: 215.2

Synthesis of (6-cyclopropylimidazo[1,2-a]pyrimidin-2-yl)methanamine. To a mixture of 2-(azidomethyl)-6-cyclopropylimidazo[1,2-a]pyrimidine (80 mg, 0.37 mmol) in MeOH (10 mL) was added PPh₃ (196 mg, 0.74 mmol) at room temperature. The mixture was stirred at 60° C. for 3 h. The reaction mixture was cooled to room temperature and concentrated in vacuo to give the crude, which was purified by preparative TLC to give the (6-cyclopropylimidazo[1,2-a]pyrimidin-2-yl)methanamine (45 mg, 64%) as a yellow solid. ESI-MS [M+H]⁺: 189.2

### Synthesis of 1-(2-(aminomethyl)-6-cyclopropylimidazo[1,2-a]pyrazin-8-yl)-3-methylimidazolidine-2,4-dione

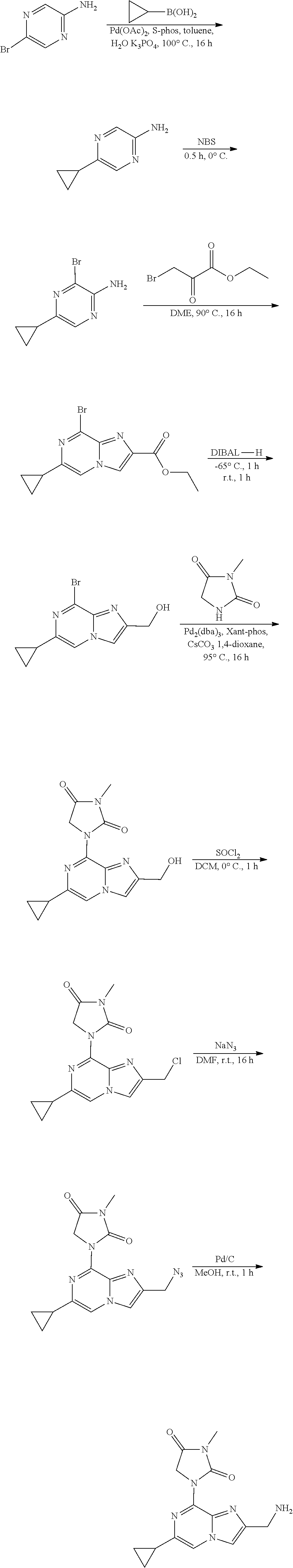

Synthesis of 5-cyclopropylpyrazin-2-amine. To a mixture of 5-bromopyrazin-2-amine (10 g, 57 mmol) in toluene/H₂O (V/V=1/1, 400 mL) were added cyclopropylboronic acid (15 g, 0.17 mol), S-phos (2.4 g, 5.8 mmol), K₃PO₄ (36 g, 0.17 mol) and Pd(OAc)₂ (1.3 g, 5.8 mmol). The mixture was stirred at 100° C. for 16 h under N₂ and then cooled to room temperature. The reaction mixture was filtered through Celite® and the filter cake was washed with DCM/MeOH (10/1, 100 mL). The filtrate was concentrated in vacuo to give the crude, which was purified by silica gel chromatography (eluent: PE/EtOAc=10/1˜ 5/1) to give 5-cyclopropylpyrazin-2-amine (5.6 g, 72%) as a yellow solid. ESI-MS [M+H]⁺: 136.1.

Synthesis of 3-bromo-5-cyclopropylpyrazin-2-amine. To a stirred solution of 5-cyclopropylpyrazin-2-amine (5.6 g, 41 mmol) in CH₃CN (40 mL) was added NBS (7.4 g, 42 mmol) at 0° C. The mixture was stirred at room temperature for 30 min. The reaction mixture was poured into water (50 mL) and extracted with EtOAc (3×50 mL). The combined organics were washed with saturated brine solution (100 mL) and dried over Na₂SO₄. The organic layers were concentrated in vacuo and purified by column chromatography on silica gel (eluent: PE/EtOAc=10/1) to give 3-bromo-5-cyclopropylpyrazin-2-amine (2.0 g, yield 23%) ESI-MS [M+H]⁺: 214.0.

Synthesis of ethyl 8-bromo-6-cyclopropylimidazo[1,2-a]pyrazine-2-carboxylate. To a mixture of 3-bromo-5-cyclopropylpyrazin-2-amine (2.0 g, 9.3 mmol) in DME (30 mL) was added ethyl 3-bromo-2-oxopropanoate (2.0 g, 10 mmol). The mixture was stirred at 90° C. for 16 h and cooled to room temperature. Then the mixture was poured into water (50 mL) and extracted with EtOAc (3×50 mL). The combined organics were washed with saturated brine solution (100 mL), dried over Na₂SO₄ and concentrated in vacuo. The residue was purified by column chromatography on silica gel (eluent: PE/EtOAc=2/1) to give ethyl 8-bromo-6-cyclopropylimidazo[1,2-a]pyrazine-2-carboxylate (1.0 g, 35%). ESI-MS [M+H]⁺: 310.0.

Synthesis of (8-bromo-6-cyclopropylimidazo[1,2-a]pyrazin-2-yl)methanol. To a mixture of 8-bromo-6-cyclopropylimidazo[1,2-a]pyrazine-2-carboxylate (1.0 g, 3.2 mmol) in THF (20 mL) was added DIBAL-H (11 mL, 11 mmol) at −65° C. The mixture was stirred at −65° C. for 1 h and then warmed to 25° C. and stirred for another 1 h. The reaction mixture was poured into ice-water (20 mL) and extracted with EtOAc (3×50 mL). The combined organics were washed with saturated brine solution (50 mL), dried over Na₂SO₄ and concentrated in vacuo to give a residue, which was purified by column chromatography on silica gel (eluent: PE/EtOAc=5/1) to give (8-bromo-6-cyclopropylimidazo[1,2-a]pyrazin-2-yl)methanol (0.49 g, yield 57%). ESI-MS [M+H]⁺: 268.0.

Synthesis of 1-(6-cyclopropyl-2-(hydroxymethyl)imidazo[1,2-a]pyrazin-8-yl)-3-methylimidazolidine-2,4-dione. To a mixture of (8-bromo-6-cyclopropylimidazo[1,2-a]pyrazin-2-yl)methanol (0.49 g, 1.8 mmol) in dioxane (15 mL) were added 3-methylimidazolidine-2,4-dione (0.83 g, 7.3 mmol), Pd₂(dba)₃ (0.34 g, 0.37 mmol), Xant-Phos (0.38 g, 0.66 mmol), Cs₂CO₃ (1.8 g, 5.5 mmol). After stirring at 90° C. for 16 h under N₂, the reaction mixture was filtered and the filter cake was washed with DCM/MeOH (10/1, 50 mL). The filtrate was concentrated in vacuo to give the crude, which was purified by column chromatography on silica gel (eluent: DCM/MeOH=30/1 -10/1) to give 1-(6-cyclopropyl-2-(hydroxymethyl)imidazo[1,2-a]pyrazin-8-yl)-3-methylimidazolidine-2,4-dione (0.40 g, 74%). ESI-MS [M+H]⁺: 302.1.

Synthesis of 1-(2-(chloromethyl)-6-cyclopropylimidazo[1,2-a]pyrazin-8-yl)-3-methylimidazolidine-2,4-dione. A mixture of 1-(6-cyclopropyl-2-(hydroxymethyl)imidazo[1,2-a]pyrazin-8-yl)-3-methylimidazolidine-2,4-dione (0.50 g, 1.7 mmol) in DCM (10 mL) was added SOCl₂ (0.98 g, 8.2 mmol) at 0° C. and stirred at 0° C. for 1 h under N₂. The mixture was concentrated in vacuo to give 1-(2-(chloromethyl)-6-cyclopropylimidazo[1,2-a]pyrazin-8-yl)-3-methylimidazolidine-2,4-dione (0.40 g, 74%) as a yellow solid. ESI-MS [M+H]⁺: 320.1.

Synthesis of 1-(2-(azidomethyl)-6-cyclopropylimidazo[1,2-a]pyrazin-8-yl)-3-methylimidazolidine-2,4-dione. To a mixture of 1-(2-(chloromethyl)-6-cyclopropylimidazo[1,2-a]pyrazin-8-yl)-3-methylimidazolidine-2,4-dione (0.40 g, 1.3 mmol) in DMF (10 mL) was added NaN₃ (0.24 g, 3.7 mmol) at 25° C. The mixture was stirred at 25° C. for 16 h under N₂. The reaction was quenched with water (10 mL) and extracted with DCM (3×20 mL). The organic layers were combined and concentrated in vacuo to give the crude product, which was purified by preparative TLC (eluent: DCM/MeOH=20/1) to 1-(2-(azidomethyl)-6-cyclopropylimidazo[1,2-a]pyrazin-8-yl)-3-methylimidazolidine-2,4-dione (0.30 g, 71%) as a yellow oil. ESI-MS [M+H]⁺: 327.1.

Synthesis of 1-(2-(aminomethyl)-6-cyclopropylimidazo[1,2-a]pyrazin-8-yl)-3-methylimidazolidine-2,4-dione. To a mixture of 1-(2-(azidomethyl)-6-cyclopropylimidazo[1,2-a]pyrazin-8-yl)-3-methylimidazolidine-2,4-dione (0.30 g, 0.92 mmol) in MeOH (6 mL) was added Pd/C (0.30 g, 0.92 mmol) at 25° C. The mixture was stirred at 25° C. for 1 h under an atmosphere of H₂. The resulting mixture was filtered through Celite® and the filter cake was washed with DCM/MeOH (10/1, 40 mL). The filtrate was concentrated in vacuo to give 1-(2-(aminomethyl)-6-cyclopropylimidazo[1,2-a]pyrazin-8-yl)-3-methylimidazolidine-2,4-dione (0.22 g, 80%) as a yellow oil which was used without further purification. ESI-MS [M+H]⁺: 301.1.

### Synthesis of 2-(chloromethyl)-6-cyclopropylimidazo[1,2-b]pyridazine

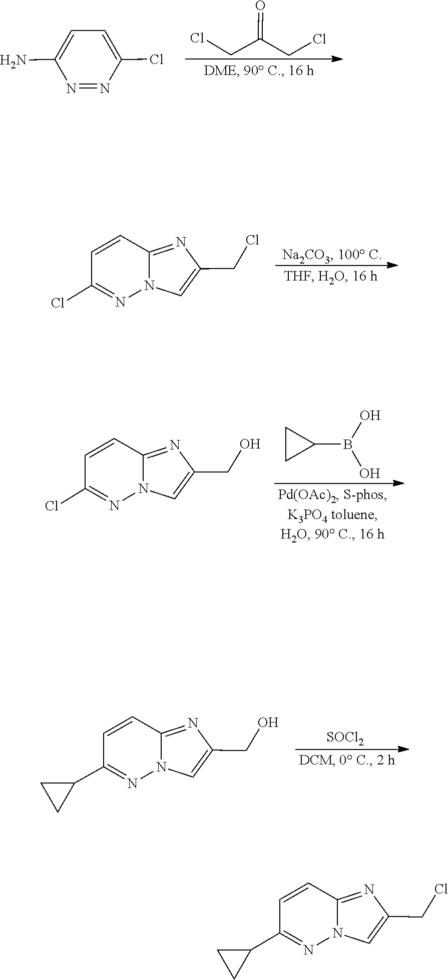

Synthesis of 6-chloro-2-(chloromethyl)imidazo[1,2-b]pyridazine. To a solution of 6-chloropyridazin-3-amine (5.2 g, 40 mmol) in DME (40 mL) was added 1,3-dichloropropan-2-one (7.2 g, 57 mmol). The mixture was stirred at 90° C. for 16 h under N₂. After cooling to room temperature, the reaction mixture was filtered through Celite® and the filter cake was washed with DCM/MeOH (10/1, 0.10 L). The filtrate was concentrated in vacuo to give the crude, which was purified by column chromatography on silica gel (eluent: PE/EtOAc=10/1˜ 2/1) to give 6-chloro-2-(chloromethyl)imidazo[1,2-b]pyridazine (3.8 g, 47%) as a solid. ESI-MS [M+H]⁺: 202.0.

Synthesis of (6-chloroimidazo[1,2-b]pyridazin-2-yl)methanol. To a solution of 6-chloro-2-(chloromethyl)imidazo[1,2-b]pyridazine (3.8 g, 19 mmol) in THF/H₂O (V/V=1/1, 0.6 L) was added Na₂CO₃ (10 g, 94 mmol). The mixture was stirred at 100° C. for 16 h under N₂. After cooling to room temperature, the reaction mixture was extracted with EtOAc (3×300 mL). The organic layers were combined and concentrated in vacuo to give the crude, which was purified by preparative TLC (eluent: DCM/MeOH=10/1) to give (6-chloroimidazo[1,2-b]pyridazin-2-yl)methanol (2.8 g, 80%) as a yellow solid. ESI-MS [M+H]⁺: 184.0.

Synthesis of (6-cyclopropylimidazo[1,2-b]pyridazin-2-yl)methanol. To a mixture of (6-chloroimidazo[1,2-b]pyridazin-2-yl)methanol (1.4 g, 7.6 mmol) in toluene/H₂O ((V/V=1/1, 56 mL) was added cyclopropylboronic acid (0.98 g, 11 mmol), S-phos (0.94 g, 2.3 mmol), K₃PO₄ (3.8 g, 18 mmol) and Pd(OAc)₂ (0.50 g, 2.2 mmol). The mixture was stirred at 90° C. for 16 h under N₂. After cooling to room temperature, the reaction mixture was filtered through Celite® and the filter cake was washed with DCM/MeOH (10/1, 0.10 L). The filtrate was concentrated in vacuo to give the crude, which was purified by column chromatography (eluent: DCM/MeOH=20/1˜ 10/1) to give (6-cyclopropylimidazo[1,2-b]pyridazin-2-yl)methanol (0.75 g, 52%) as a yellow oil. ESI-MS [M+H]⁺: 190.1.

Synthesis of 2-(chloromethyl)-6-cyclopropylimidazo[1,2-b]pyridazine. To a mixture of (6-cyclopropylimidazo[1,2-b]pyridazin-2-yl)methanol (0.75 g, 4.0 mmol) in DCM (20 mL) was added SOCl₂ (2.0 g, 17 mmol) at 0° C. The mixture was stirred at 0° C. for 2 h under N₂. The reaction mixture was concentrated in vacuo to give 2-(chloromethyl)-6-cyclopropylimidazo[1,2-b]pyridazine (1 g, quant) as a yellow solid. ESI-MS [M+H]⁺: 208.0.

### Synthesis of 6-cyclopropylimidazo[1,2-b]pyridazin-2-yl)methanamine

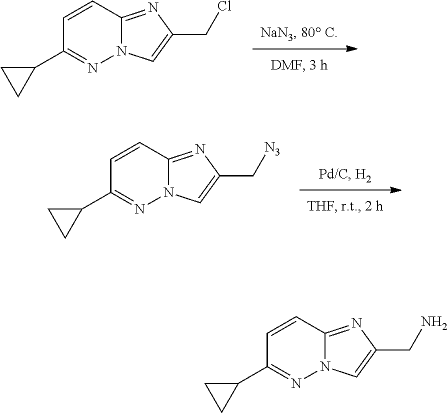 [SMILES: ClCc1cn2nc(C3CC3)ccc2n1 ; [N-]=[N+]=NCc1cn2nc(C3CC3)ccc2n1 ; NCc1cn2nc(C3CC3)ccc2n1]

Synthesis of 2-(azidomethyl)-6-cyclopropylimidazo[1,2-b]pyridazine. A mixture of 2-(chloromethyl)-6-cyclopropylimidazo[1,2-b]pyridazine (580 mg, crude) and NaN₃ (310 mg, 4.74 mmol) in DMF (6 mL) was stirred at 80° C. for 3 h under N₂. The reaction mixture was washed with water (20 mL), extracted with EtOAc (3×40 mL). The combined organic layers were washed with brine, dried over Na₂SO₄, and concentrated in vacuo to give the crude, which was purified by preparative TLC (eluent: DCM/MeOH=20/1) to give 2-(azidomethyl)-6-cyclopropylimidazo[1,2-b]pyridazine (550 mg, crude) as yellow oil. ESI-MS [M+H]⁺: 215.1.

Synthesis of (6-cyclopropylimidazo[1,2-b]pyridazin-2-yl)methanamine. A mixture of 2-(azidomethyl)-6-cyclopropylimidazo[1,2-b]pyridazine (550 mg, 2.0 mmol) and Pd/C (1 g) in THF (6 mL) was stirred at room temperature under H₂ atmosphere. The mixture was filtered through Celite® and the filter cake was washed with DCM/MeOH (10/1, 100 mL). The filtrate was concentrated in vacuo to give (6-cyclopropylimidazo[1,2-b]pyridazin-2-yl)methanamine (230 mg, crude) as a yellow oil. ESI-MS [M+H]⁺: 189.1.

### Synthesis of 6-(chloromethyl)-3-cyclopropylquinoline

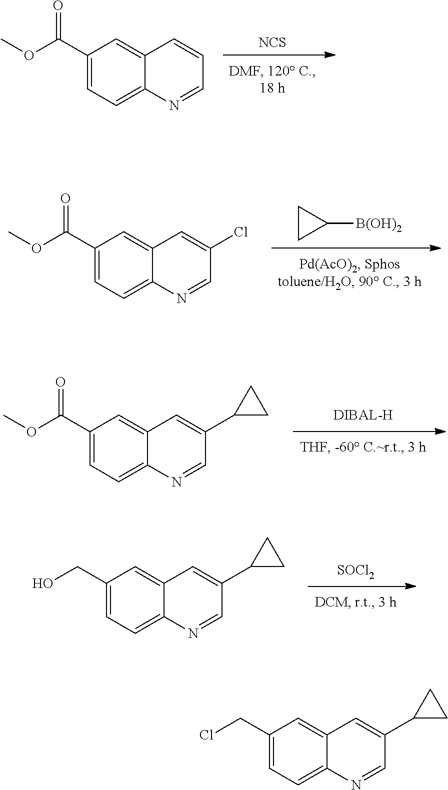

Synthesis of methyl 3-chloroquinoline-6-carboxylate. To a solution of methyl quinoline-6-carboxylate (10 g, 53.5 mmol) in DMF (150 mL) was added NCS (21 g, 160.5 mmol) and the reaction mixture was stirred at 120° C. for 18 h under N₂. After cooling to room temperature, the reaction was quenched with NaHCO₃ (sat. aq., 500 mL) and extracted with EtOAc (150 mL×3). The combined organic layers were washed with brine (50 mL×3), dried over Na₂SO₄ and concentrated in vacuo to give the crude, which was purified by column chromatography (eluent: MeOH/DCM=1/20) to give methyl 3-chloroquinoline-6-carboxylate (5.0 g, 42% yield) as an off-white solid. ESI-MS [M+H]⁺: 222.1.

Synthesis of methyl 6-cyclopropylquinoline-3-carboxylate. A mixture of methyl 6-chloroquinoline-3-carboxylate (5.0 g, 22.6 mmol), cyclopropylboronic acid (2.9 g, 33.9 mmol), Pd(OAc)₂ (520 mg, 2.3 mmol), S-Phos (943 mg, 2.3 mmol) and K₃PO₄ (14.4 g, 67.8 mmol) in toluene (100 mL) and H₂O (10 mL) was stirred at 90° C. for 3 h under N₂. After cooling to room temperature, the reaction mixture was filtered through Celite® and the filter cake was washed with DCM/MeOH (10/1, 200 mL). The filtrate was concentrated in vacuo to give the crude, which was purified by column chromatography (eluent: MeOH/DCM=0 to 5%) to give methyl 3-cyclopropylquinoline-6-carboxylate (4.6 g, 90% yield) as a yellow solid. ESI-MS [M+H]⁺: 228.1.

Synthesis of (6-cyclopropylquinolin-3-yl)methanol. To a mixture of methyl 6-cyclopropylquinoline-3-carboxylate (1.5 g, 6.6 mmol) in THF (20 mL) was added DIBALH (13.2 mL, 13.2 mmol, 1 M in THF) slowly at −60° C. under N₂. The reaction mixture was then slowly raised to room temperature and then stirred at room temperature for 3 h. The reaction was quenched with Na₂SO₄·10H₂O, filtered through Celite® and the filter cake was washed with DCM/MeOH (10/1, 200 mL). The filtrate was concentrated in vacuo to give the crude, which was purified by column chromatography (eluent: MeOH/DCM=0 to 8%) to give (3-cyclopropylquinolin-6-yl)methanol (900 mg, 69% yield) as a yellow oil. ESI-MS [M+H]⁺: 200.2.

Synthesis of 6-(chloromethyl)-3-cyclopropylquinoline. To a mixture of (6-cyclopropylquinolin-3-yl)methanol (900 mg, 4.5 mmol) in DCM (10 mL) was added SOCl₂ (3.27 g, 27.5 mmol) slowly. The reaction mixture was then stirred at room temperature for 3 h. The reaction was quenched with NaHCO₃ (sat. aq., 50 mL) and extracted with DCM (50 mL×3). The combined organic layers were washed with brine (50 mL), dried over Na₂SO₄ and concentrated in vacuo to give the crude, which was purified by column chromatography (eluent: MeOH/DCM=0 to 5%) to give 6-(chloromethyl)-3-cyclopropylquinoline (700 mg, 72% yield) as a yellow solid. ESI-MS [M+H]⁺: 218.1.

### Synthesis of benzyl (2R,4S)-4-((tert-butyldimethylsilyl)oxy)-2-(2-chloroacetyl)pyrrolidine-1-carboxylate

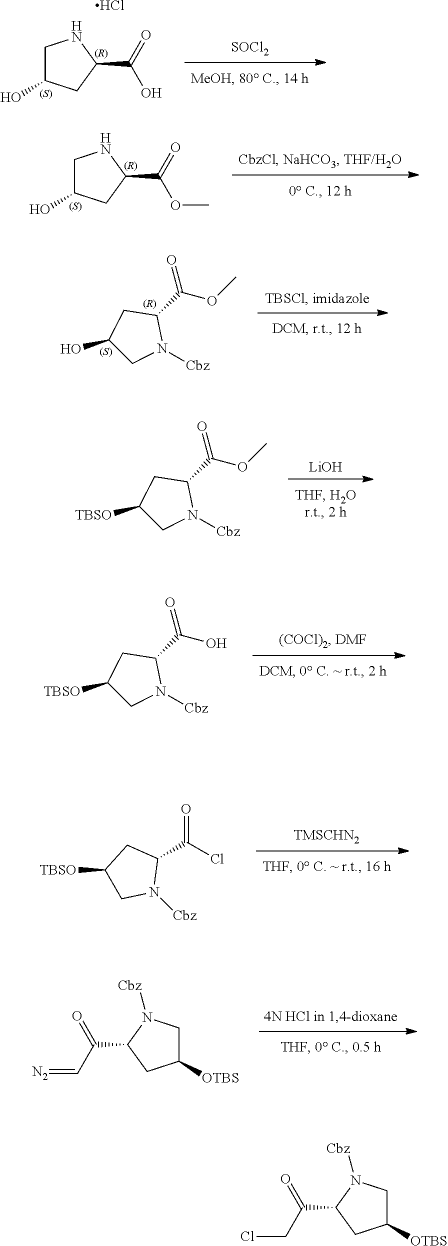

Synthesis of methyl (2R,4S)-4-hydroxypyrrolidine-2-carboxylate. To a solution of (2R,4S)-4-hydroxypyrrolidine-2-carboxylic acid hydrochloride (5 g, 29.8 mmol) in MeOH (50 mL) was added SOCl₂ (10.6 g, 89.4 mmol) slowly at 0° C. After stirring at 80° C. for 12 h, the reaction was concentrated in vacuo to give methyl (2R,4S)-4-hydroxypyrrolidine-2-carboxylate (4 g, crude) as a grey solid. ESI-MS [M+H]⁺: 146.2.

Synthesis of 1-benzyl 2-methyl (2R,4S)-4-hydroxypyrrolidine-1,2-dicarboxylate. To a solution of methyl (2R,4S)-4-hydroxypyrrolidine-2-carboxylate (4 g, 27.5 mmol) in THF (80 mL) was added saturated aqueous NaHCO₃ (60 ml), followed by CbzCl (5.6 g, 33.1 mmol) at 0° C. The reaction mixture was stirred at 0° C. for another 1 h and then extracted with EtOAc (3×50 mL). The combined organic layers were washed with brine, dried over Na₂SO₄ and concentrated in vacuo to give the crude, which was purified by silica gel chromatography (EtOAc/PE=1/5) to give 1-benzyl 2-methyl (2R,4S)-4-hydroxypyrrolidine-1,2-dicarboxylate (5.8 g, 75%) as yellow oil. ESI-MS [M+H]⁺: 280.1.

Synthesis of 1-benzyl 2-methyl (2R,4S)-4-((tert-butyldimethylsilyl)oxy)pyrrolidine-1,2-dicarboxylate, A mixture of 1-benzyl 2-methyl (2R,4S)-4-hydroxypyrrolidine-1,2-dicarboxylate (4 g, 14.3 mmol), TMSCl (2.3 g, 21.5 mmol), imidazole (1.9 g, 28.6 mmol) in DCM (40 mL) was stirred at room temperature for 12 h. The reaction was washed with water (50 mL) then extracted with EtOAc (3×50 mL). The combined organic layers were washed with brine, dried over Na₂SO₄, filtered and concentrated in vacuo to give the crude, which was purified by silica gel chromatography (EtOAc/PE=1/3) to give 1-benzyl 2-methyl (2R,4S)-4-((tert-butyldimethylsilyl)oxy)pyrrolidine-1,2-dicarboxylate (4 g, 71%) as yellow oil. ESI-MS [M+H]⁺: 394.1.

Synthesis of (2R,4S)-1-((benzyloxy)carbonyl)-4-((tert-butyldimethylsilyl)oxy)pyrrolidine-2-carboxylic acid. To a solution of 1-benzyl 2-methyl (2R,4S)-4-((tert-butyldimethylsilyl)oxy)pyrrolidine-1,2-dicarboxylate (4 g, 10.2 mmol) in THF (40 mL) was added LiOH (1.25 g, 30.6 mmol) and water (4 mL). The reaction mixture was stirred at room temperature for 2 h and then concentrated in vacuo to remove THF. The pH of the resulting residue was adjusted to 3-4 with HCl (1.0 M) then the mixture was extracted with EtOAc (3×50 mL). The combined organic layers were washed with brine, dried over Na₂SO₄, filtered and concentrated in vacuo to give the crude (2R,4S)-1-((benzyloxy)carbonyl)-4-((tert-butyldimethylsilyl)oxy)pyrrolidine-2-carboxylic acid (3.3 g, 87%) as yellow oil. ESI-MS [M+H]⁺: 394.1.

Synthesis of benzyl(2R,4S)-4-((tert-butyldimethylsilyl)oxy)-2-(chlorocarbonyl)pyrrolidine-1-carboxylate. To a mixture of (2R,4S)-1-((benzyloxy)carbonyl)-4-((tert-butyldimethylsilyl)oxy)pyrrolidine-2-carboxylic acid (5.0 g, 13 mmol) in DCM (0.10 L) was added (COCl)₂ (2.0 g, 16 mmol) and DMF (0.010 mL) at 0° C. The reaction mixture was stirred at room temperature for 2 h under N₂ and then was concentrated in vacuo to give benzyl (2R,4S)-4-((tert-butyldimethylsilyl)oxy)-2-(chlorocarbonyl)pyrrolidine-1-carboxylate (5.2 g, quant) as a yellow oil, which was used without further purification. ESI-MS [M+H]⁺: 398.1.

Synthesis of benzyl (2R,4S)-4-((tert-butyldimethylsilyl)oxy)-2-(2-diazoacetyl)pyrrolidine-1-carboxylate. To a mixture of give benzyl (2R,4S)-4-((tert-butyldimethylsilyl)oxy)-2-(chlorocarbonyl)pyrrolidine-1-carboxylate (7.3 g, 18 mmol) in THF (0.10 L) was added TMSCHN₂ (4.4 g, 39 mmol) at 0° C. The reaction mixture was stirred at room temperature for 16 h under N₂ and then concentrated in vacuo to give benzyl (2R,4S)-4-((tert-butyldimethylsilyl)oxy)-2-(2-diazoacetyl)pyrrolidine-1-carboxylate (7.4 g, quant) as a yellow oil, which was used without further purification. ESI-MS [M+H]⁺: 404.2.

Synthesis of benzyl (2R,4S)-4-((tert-butyldimethylsilyl)oxy)-2-(2-chloroacetyl) pyrrolidine-1-carboxylate. To a mixture of benzyl (2R,4S)-4-((tert-butyldimethylsilyl)oxy)-2-(2-diazoacetyl)pyrrolidine-1-carboxylate (7.4 g, 18 mmol) in THF (0.1 L) was added HCl (4 N in dioxane, 5.5 mL, 22 mmol). The reaction mixture was stirred at room temperature for 0.5 h under N₂. The mixture was concentrated in vacuo to give the crude, which was purified by column chromatography on silica gel (eluent: DCM/MeOH=50/1˜ 20/1) to give benzyl (2R,4S)-4-((tert-butyldimethylsilyl)oxy)-2-(2-chloroacetyl)pyrrolidine-1-carboxylate (4.4 g, 59%) as a yellow solid. ESI-MS [M+H]⁺: 412.1.

### Synthesis of tert-butyl (S—4-((tert-butyldimethylsilyl)oxy)-2-oxopyrrolidine-1-carboxylate

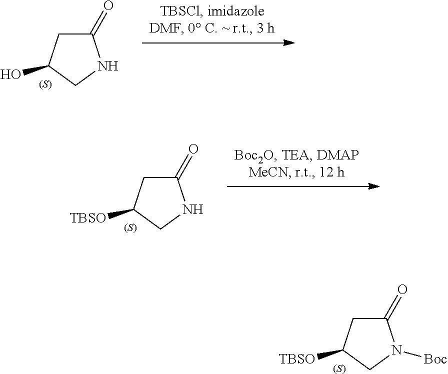

Synthesis of (S)-4-((tert-butyldimethylsilyl)oxy)pyrrolidin-2-one. To a solution of (S)-4-hydroxypyrrolidin-2-one (7.2 g, 71.2 mmol) in DMF (50 mL) was added imidazole (7.3 g, 107 mmol) and TBSCl (11.8 g, 78.4 mmol) subsequently at 0° C. The resulting mixture was stirred at room temperature for 3 h. The reaction was quenched by water (100 mL). The resulting precipitate was collected by filtration and then dried in vacuo to give (S)-4-((tert-butyldimethylsilyl)oxy)pyrrolidin-2-one (15 g, crude) as a white solid. ESI-MS [M+H]⁺: 216.1

Synthesis of tert-butyl (S)-4-((tert-butyldimethylsilyl)oxy)-2-oxopyrrolidine-1-carboxylate. A solution of (S)-4-((tert-butyldimethylsilyl)oxy)pyrrolidin-2-one (15.3 g, 71 mmol), DMAP (4.3 g, 35.5 mmol), Boc₂O (31 g, 142 mmol) and Et₃N (14.4 g, 142 mmol) in MeCN (100 mL) was stirred at room temperature for 13 h. The reaction was quenched by water (100 mL), the resulting precipitate was collected by filtration and then dried in vacuo. The residue was purified by column chromatography on silica gel, eluting with 0˜15% EtOAc in PE to give tert-butyl (S)-4-((tert-butyldimethylsilyl)oxy)-2-oxopyrrolidine-1-carboxylate as a white solid (7 g, 31%). ESI-MS [M+H]⁺: 316.2.

### Example 1

### Synthesis of rac-(1S*,2S*)—N-(5-((4-((1H-pyrazol-1-yl)methyl)benzyl)oxy)pyridazin-3-yl)-2-(5-chloropyridin-3-yl)cyclopropane-1-carboxamide (I-1)

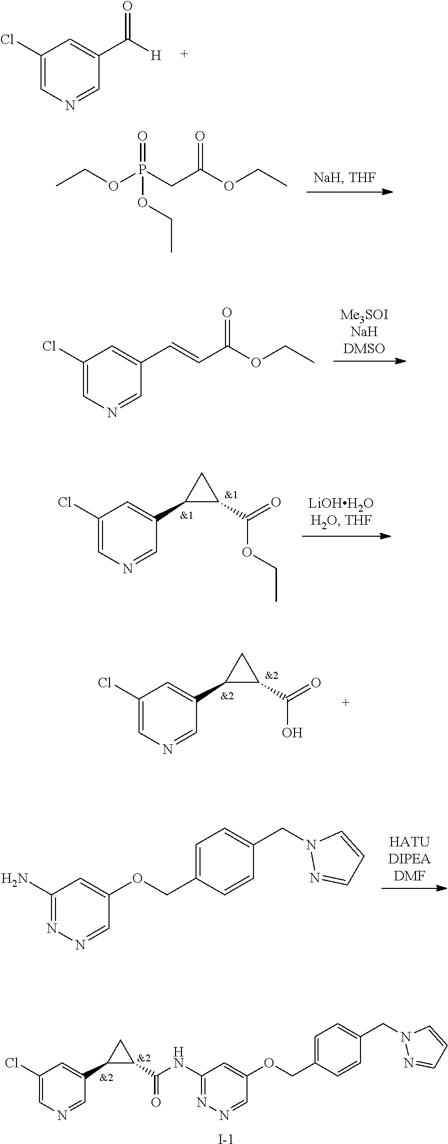

Synthesis of ethyl (E)-3-(5-chloropyridin-3-yl)acrylate. A solution of triethylphosphonoacetate (3.08 mL, 15.54 mmol) in THF (30 mL) was added dropwise to a salt/ice bath cooled suspension of NaH (60% in mineral oil, 0.85 g, 21.19 mmol) in THF (30 mL) at 0° C. After 45 min. a solution of 5-chloro-pyridine-3-carbaldehyde (2.00 g, 14.13 mmol) was added dropwise. The mixture was allowed to warm to room temperature and, after 2 h, water (100 mL) was added. The mixture was extracted with EtOAc (3×100 mL), the organic phases were combined, passed through a phase separator cartridge and concentrated in vacuo to give a dark yellow solid. This solid was triturated with di-isopropyl ether to give the title compound (1.92, 64%) as a pale yellow solid. ESI-MS (M+H)+: 212, ¹H NMR (400 MHz, DMSO) δ 8.91 (s, 1H), 8.69 (d, J=1.3 Hz, 1H), 8.46 (s, 1H), 7.73 (d, J=16.2 Hz, 1H), 6.96 (d, J=16.2 Hz, 1H), 4.27 (q, J=7.0 Hz, 2H), 1.32 (t, J=7.1 Hz, 3H).

Synthesis of ethyl rac-(S*,2S*)-2-(5-chloropyridin-3-yl)cyclopropane-1-carboxylate. Sodium hydride (60% in mineral oil, 0.28 g, 7.09 mmol) was added portion-wise to a suspension of trimethylsulfoxonium iodide (2.08 g, 9.46 mmol) in DMSO (10 mL) at room temperature, after 25 min. a solution of ethyl (E)-3-(5-chloropyridin-3-yl)acrylate (1.00 g, 4.73 mmol) in DMSO (10 mL) was added dropwise. Mixture was stirred at room temperature for 3 h then treated with ammonium chloride (aq. sat., 5 mL). The mixture was purified using C18 silica gel reverse phase chromatography eluting with a gradient of 5-95% MeCN/Water (10 mMol NH₄HCO₃) to give the title compound (602 mg, 56%). ¹H NMR (400 MHz, DMSO) δ 8.52 (d, J=2.1 Hz, 1H), 8.51 (d, J=2.3 Hz, 1H), 7.81-7.79 (m, 1H), 4.16 (q, J=6.9 Hz, 2H), 2.61-2.57 (m, 1H), 2.22-2.16 (m, 1H), 1.58-1.53 (m, 2H), 1.26 (t, J=7.2 Hz, 3H).

Synthesis of rac-(1S*,2S*)-2-(5-chloropyridin-3yl)cyclopropane-1-carboxylic acid. Ethyl rac-(1S*,2S*)-2-(5-chloropyridin-3-yl)cyclopropane-1-carboxylate (602 mg, 2.21 mmol) in THF (2 mL) was added to an aqueous solution of lithium hydroxide (93 mg, 2.21 mmol) and the mixture was stirred at room temperature for 48 h. The reaction mixture was acidified using HCl (1M aq., 5 mL) and extracted with EtOAc (3×10 mL). The organic phase was passed through a phase separator cartridge and concentrated in vacuo to give the title compound (540 mg, 91%). ESI-MS (M+H)+: 198, ¹H NMR (400 MHz, DMSO) δ 12.45-12.45 (m, 1H), 8.47 (d, J=1.8 Hz, 1H), 8.45 (d, J=2.2 Hz, 1H), 7.74 (dd, J=2.1, 2.1 Hz, 1H), 2.50-2.46 (m, 1H), 2.02-1.97 (m, 1H), 1.50-1.45 (m, 2H).

Synthesis of rac-(1S*,2S*)—N-(5-((4-((1H-pyrazol-1-yl)methyl)benzyl)oxy)pyridazin-3-yl)-2-(5-chloropyridin-3-yl)cyclopropane-1-carboxamide. rac-(1S*,2S*)-2-(5-chloropyridin-3-yl)cyclopropane-1-carboxylic acid (50 mg, 0.25 mmol), HATU (116 mg, 0.30 mmol) and DIPEA (0.07 mL, 0.38 mmol) were stirred together in DMF (1 mL) for 1 hour. 5-((4-((1H-pyrazol-1-yl)methyl)benzyl)oxy)pyridazin-3-amine (68 mg, 0.24 mmol) was then added and the mixture was stirred for 18 h. The mixture was diluted with water. The resulting precipitate was collected by filtration, washed with di-isopropyl ether then EtOAc then dried to give the title compound as a beige solid (41 mg, 35%). ESI-MS (M+H)+: 461.4, ¹H NMR (400 MHz, DMSO) δ 11.37 (s, 1H), 8.78 (d, J=2.8 Hz, 1H), 8.49 (d, J=2.1 Hz, 2H), 8.03 (d, J=2.6 Hz, 1H), 7.84-7.83 (m, 1H), 7.78-7.76 (m, 1H), 7.48-7.43 (m, 3H), 7.27-7.24 (m, 2H), 6.29-6.27 (m, 1H), 5.36 (s, 2H), 5.24 (s, 2H), 1.63-1.54 (m, 2H). 2H missing, presumed under DMSO peak.

### Example 2

Synthesis of rac-(1S*,2S*)-2-(3-chlorophenyl)-N-(5-((2-phenylthiazol-4-yl)methoxy)pyridazin-3-ylcyclopropane-1-carboxamide (I-2)

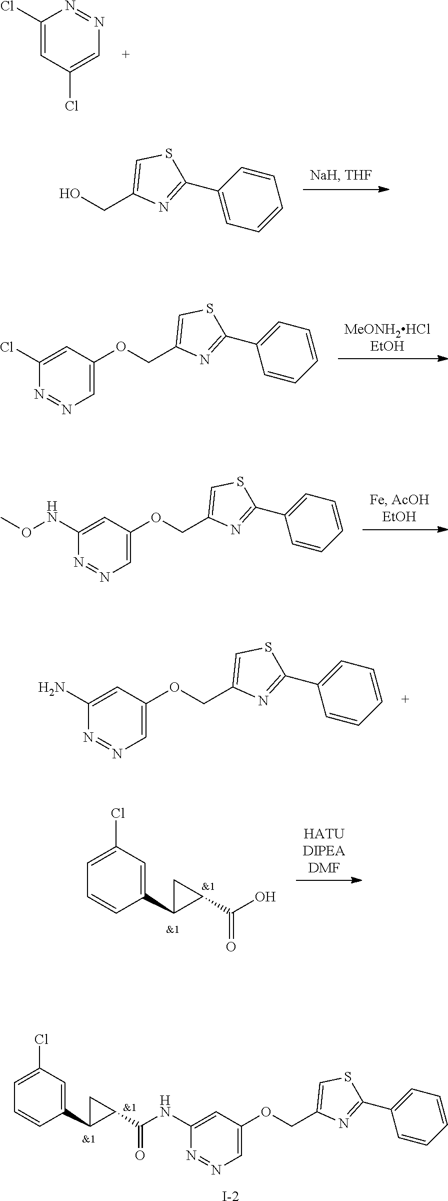

Synthesis of 4-(((6-chloropyridazin-4-yl)oxy)methyl)-2-phenylthiazole. Sodium hydride (60% in mineral oil, 0.251 g, 5.27 mmol) was added portion-wise to a cooled, stirred solution of (2-phenylthiazol-4-yl)methanol (1.00 g, 5.23 mmol) at 0° C. in DMF (10 mL). After 15 min, 3,5-dichloropyridazine (0.857 mg, 5.75 mmol) was added portion-wise over 15 min then the mixture was allowed to warm to room temperature and stirred for 20 h. Reaction mixture was poured into water (100 mL) and extracted with EtOAc (3×100 mL), the organic phases were combined, dried over MgSO₄, filtered and concentrated in vacuo to give a gum. The gum was triturated with Et₂O to give the title compound (1.04 g, 63%). ESI-MS (M+H)+: 304.0, ¹H NMR (400 MHz, DMSO) δ 9.17 (d, J=2.4 Hz, 1H), 8.01-7.99 (m, 3H), 7.83 (d, J=2.4 Hz, 1H), 7.59-7.56 (m, 3H), 5.52 (s, 2H).

Synthesis of O-methyl-N-(5-((2-phenylthiazol-4-yl)methoxy)pyridazin-3-yl)hydroxylamine. Methyl hydroxylamine·HCl (2.464 g, 29.50 mmol) was added to a stirred solution of 4-(((6-chloropyridazin-4-yl)oxy)methyl)-2-phenylthiazole (0.448 g, 1.47 mmol) in EtOH (10 mL) and the mixture was heated to 85° C. for 3 h. The reaction mixture was diluted with EtOH, filtered and concentrated in vacuo. The residue was dissolved in NaHCO₃ (aq. sat., 50 mL) and extracted with DCM (3×50 mL). The organic phases were combined, filtered through a phase separator cartridge, and concentrated in vacuo to give the title compound (459 mg, 99%) as a yellow gum which was used without further purification. ESI-MS (M+H)+: 315.0.

Synthesis of 5-((2-phenylthiazol-4-yl)methoxy)pyridazin-3-amine. Acetic acid (2.25 mL) was added to a stirred mixture of O-methyl-N-(5-((2-phenylthiazol-4-yl)methoxy)pyridazin-3-yl)hydroxylamine (0.45 g, 1.43 mmol), Fe (0.4 g, 7.16 mmol), EtOH (15 mL) and heated to 60° C. for 2 h. The reaction mixture was filtered through Celite® and the filtrate was concentrated in vacuo to give an orange residue. The residue was purified by silica gel column chromatography, eluting with a gradient of 0-15% (7N NH₃ in MeOH) in DCM to give the title compound (121 mg, 37%) ESI-MS (M+H)+: 285.1, ¹H NMR (400 MHz, DMSO) δ 8.28 (d, J=2.6 Hz, 1H), 7.99-7.95 (m, 2H), 7.84 (s, 1H), 7.54-7.51 (m, 3H), 6.39 (d, J=2.6 Hz, 1H), 6.27 (s, 2H), 5.28 (s, 2H).

Synthesis of rac-(1S*,2S*)-2-(3-chlorophenyl)-N-(5-((2-phenylthiazol-4-yl)methoxy)pyridazin-3-yl)cyclopropane-1-carboxamide. Using a similar procedure to that used for rac-(1S*,2S*)—N-(5-((4-((1H-pyrazol-1-yl)methyl)benzyl)oxy)pyridazin-3-yl)-2-(5-chloropyridin-3-yl)cyclopropane-1-carboxamide, the title compound was synthesised from rac-(1S*,2S*)-2-(3-chlorophenyl)cyclopropane-1-carboxylic acid and 5-((2-phenylthiazol-4-yl)methoxy)pyridazin-3-amine (5 mg, 6%). ESI-MS (M+H)+: 463.3, 1H NMR (400 MHZ, DMSO) δ 11.37 (s, 1H), 8.84 (d, J=2.8 Hz, 1H), 8.15 (d, J=2.8 Hz, 1H), 7.99-7.95 (m, 2H), 7.89 (s, 1H), 7.53-7.50 (m, 3H), 7.36-7.31 (m, 1H), 7.29-7.25 (m, 2H), 7.19-7.17 (m, 1H), 5.40 (s, 2H), 2.48-2.41 (m, 2H), 1.57-1.45 (m, 2H).

The compounds in Table 6 were prepared in a similar manner to rac-(1S*,2S*)-2-(3-chlorophenyl)-N-(5-((2-phenylthiazol-4-yl)methoxy)pyridazin-3-yl)cyclopropane-1-carboxamide from rac-(1S*,2S*)-2-(3-chlorophenyl)cyclopropane-1-carboxylic acid and the indicated coupling partner.

TABLE 6

| | | Coupling | |
|---|---|---|---|
| EG | Compound | Partner | Analytical Data |
| 3 | 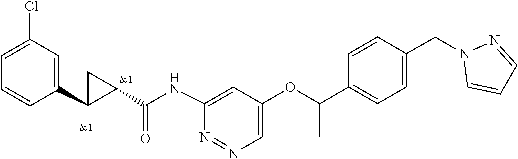 [SMILES: CC(Oc1cnnc(NC(=O)[C@H]2C[C@@H]2c2cccc(Cl)c2)c1)c1ccc(Cn2cccn2)cc1] | 5-(1-(4-((1H- pyrazol-1- yl)methyl)phe- nyl)ethoxy) pyridazin-3- amine | ESI-MS (M + H)+: 474.3, Purity: 99.1%, ¹H NMR (400 MHz, DMSO) δ 11.25 (s, 1H), 8.70 (d, J = 2.5 Hz, 1H), 7.88 (d, J = 2.5 Hz, 1H), 7.82 - 7.80 (m, 1H), 7.45 - 7.44 (m, 1H), 7.41 - 7.37 (m, 2H), 7.35 - 7.30 (m, 1H), 7.27 - 7.24 (m, 2H), 7.21 - 7.15 (m, 3H), 6.26 (t, J = 2.0 Hz, 1H), 5.69 - 5.63 (m, 1H), 5.31 |
| | rac-(1S*,2S*)-N-(5-(1-(4-((1H- | | (s, 2H), 2.47 - 2.36 (m, 2H), |
| | pyrazol-1- | | 1.57 (d, J = 6.3 Hz, 3H), 1.53 - |
| | yl)methyl)phenyl)ethoxy)pyridazin-3- | | 1.41 (m, 2H). |
| | yl)-2-(3-chlorophenyl)cyclopropane-1- | | |
| | carboxamide (I-3) | | |
| 4 | 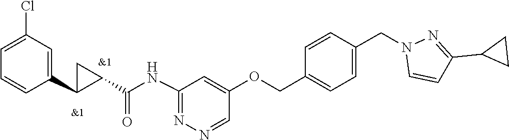 [SMILES: O=C(Nc1cc(OCc2ccc(Cn3ccc(C4CC4)n3)cc2)cnn1)[C@H]1C[C@@H]1c1cccc(Cl)c1] | 5-((4-((3- cyclopropyl- 1H-pyrazol- 1- yl)methyl)ben- zyl)oxy)py- ridazin-3- amine | ESI-MS (M + H)+: 500.3, Purity: 99.7%, ¹H NMR (400 MHz, DMSO) δ 11.28 (s, 1H), 8.69 (d, J = 2.6 Hz, 1H), 7.95 (d, J = 2.8 Hz, 1H), 7.58 (d, J = 2.3 Hz, 1H), 7.39 - 7.35 (m, 2H), 7.29 - 7.24 (m, 1H), 7.21 - 7.20 (m, 2H), 7.17 - 7.14 (m, 2H), 7.12 - 7.09 (m, 1H), 5.87 (d, |
| | rac-(1S*,2S*)-2-(3-chlorophenyl)-N- | | J = 2.1 Hz, 1H), 5.15 (s, 4H), |
| | (5-((4-((3-cyclopropyl-1H-pyrazol-1- | | 2.41 - 2.33 (m, 2H), 1.81 - 1.73 |
| | yl)methyl)benzyl)oxy)pyridazin-3- | | (m, 1H), 1.49 - 1.37 (m, 2H), |
| | yl)cyclopropane-1-carboxamide (I-4) | | 0.78 - 0.72 (m, 2H), 0.54 - 0.50 |
| | | | (m, 2H). |

### Example 5

### Synthesis of rac-(1S*2S*)—N-(5-((4-((1H-pyrazol-1-yl)methyl)benzyl)oxy)pyridazin-3-yl)-2-(2-chloropyridin-3-yl)cyclopropane-1-carboxamide (1-5)

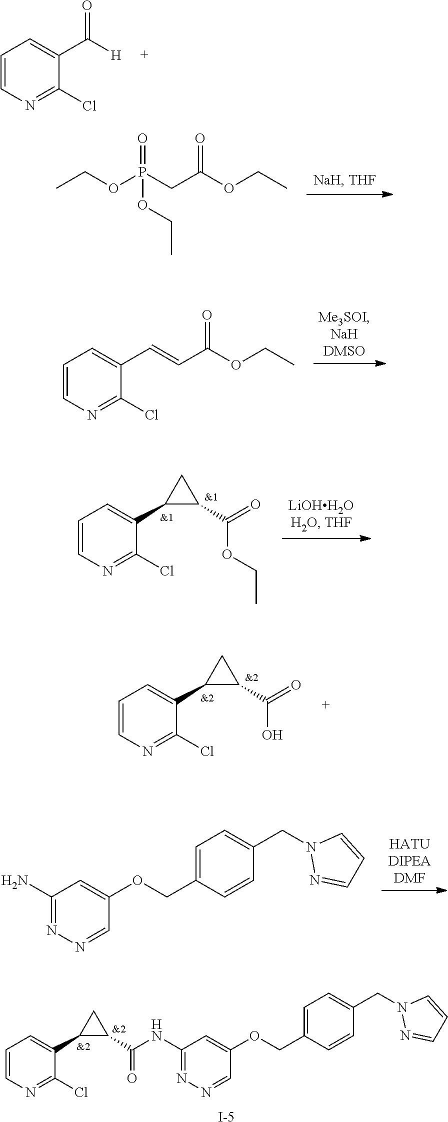

Synthesis of ethyl (E)-3-(2-chloropyridin-3-yl)acrylate. A solution of triethylphosphonoacetate (1.54 mL, 7.77 mmol) in THF (15 mL) was added dropwise to a salt/ice cooled suspension of NaH (60% in mineral oil, 0.42 g, 10.60 mmol) in THF (20 mL). The mixture was stirred for 45 min then a solution of 2-chloro-pyridine-3-carbaldehyde (2.00 g, 14.13 mmol) in THF (20 mL) was added dropwise. The mixture was allowed to warm to room temperature then after 2 h, water (100 mL) was added. The mixture was extracted with EtOAc (3×100 mL), the organic phases were combined passes through a phase separator cartridge and concentrated in vacuo to give a residue. The mixture was purified using silica gel C18 reverse phase chromatography eluting with 5-60% MeCN/Water (10 mMol NH₄HCO₃) followed by purification by column chromatography on silica gel, eluting with a gradient of 0-100% ethyl acetate/hexane to give the title compound (1.07 g, 72%). ¹H NMR (400 MHz, DMSO) δ 8.46 (dd, J=1.9, 4.6 Hz, 1H), 8.41 (dd, J=1.8, 7.7 Hz, 1H), 7.82 (d, J=17.0 Hz, 1H), 7.52 (dd, J=4.7, 7.8 Hz, 1H), 6.83 (d, J=18.1 Hz, 1H), 4.24 (q, J=7.1 Hz, 2H), 1.28 (t, J=7.1 Hz, 3H).

Synthesis of ethyl rac-(1S*,2S*)-(2-chloropyridin-3-yl)cyclopropane-1-carboxylate. Sodium hydride (60% in mineral oil, 0.30 g, 7.59 mmol) was added portion-wise to a suspension of trimethylsulfoxonium iodide (2.23 g, 10.12 mmol) at room temperature. After 25 min. a solution of ethyl (E)-3-(2-chloropyridin-3-yl)acrylate 1 g, 4.73 mmol) in DMSO (10 mL) was added dropwise. After 3 h at room temperature, ammonium chloride (aq. sat., 2.55 mL) was added and the mixture was purified using C18 reverse phase chromatography eluting with a gradient of 5-95% MeCN /water (10 mMol NH₄HCO₃) to give the title compound (388 mg, 34%). ESI-MS (M+H)+: 226.0, ¹H NMR (400 MHz, DMSO) δ 8.29 (dd, J=1.8, 4.8 Hz, 1H), 7.66 (dd, J=1.7, 7.7 Hz, 1H), 7.38 (dd, J=4.6, 7.7 Hz, 1H), 4.15 (q, J=7.1 Hz, 2H), 2.60-2.54 (m, 1H), 2.02-1.96 (m, 1H), 1.56-1.48 (m, 2H), 1.22 (t, J=7.1 Hz, 3H).

Synthesis of rac-(1S*,2S*)-(2-chloropyridin-3yl)cyclopropane-1-carboxylic acid. Ethyl rac-(1S*,2S*)-(2-chloropyridin-3-yl)cyclopropane-1-carboxylate (338 mg, 1.72 mmol) in THF (1.72 mL) was added to an aqueous solution of lithium hydroxide (79 mg, 1.89 mmol) and stirred at room temperature for 18 h. The reaction mixture was acidified using HCl (1M aq., 2 mL) and extracted with EtOAc (3×5 mL). The organic phase was passed through a phase separator cartridge and concentrated in vacuo to give the title compound (321 mg, 95%) as a yellow solid. ESI-MS (M+H)+: 198.0, ¹H NMR (400 MHz, DMSO) δ 12.45 (s, 1H), 8.29 (dd, J=1.8, 4.8 Hz, 1H), 7.64 (dd, J=1.9, 7.4 Hz, 1H), 7.38 (dd, J=4.5, 7.5 Hz, 1H), 2.57-2.53 (m, 1H), 1.87-1.82 (m, 1H), 1.51-1.45 (m, 2H).

Synthesis of rac-(1S*,2S*)—N-(5-((4-((1H-pyrazol-1-yl)methyl)benzyl)oxy)pyridazin-3-yl)-2-(2-chloropyridin-3-yl)cyclopropane-1-carboxamide. Using a similar procedure to that used for rac-(1S*,2S*)—N-(5-((4-((1H-pyrazol-1-yl)methyl)benzyl)oxy)pyridazin-3-yl)-2-(5-chloropyridin-3-yl)cyclopropane-1-carboxamide, the title compound was synthesised from rac-(1S*,2S*)-(2-chloropyridin-3-yl)cyclopropane-1-carboxylic acid and 5-((4-((1H-pyrazol-1-yl)methyl)benzyl)oxy)pyridazin-3-amine (180 mg, 68%). ESI-MS (M+H)+: 461.3, ¹H NMR (400 MHz, DMSO) δ 11.44 (s, 1H), 8.78 (d, J=2.6 Hz, 1H), 8.33-8.30 (m, 1H), 8.06 (d, J=2.8 Hz, 1H), 7.83 (d, J=1.9 Hz, 1H), 7.72-7.68 (m, 1H), 7.47-7.41 (m, 4H), 7.27-7.24 (m, 2H), 6.29-6.27 (m, 1H), 5.36 (s, 2H), 5.25 (s, 2H), 2.61-2.55 (m, 1H), 2.40-2.33 (m, 1H), 1.59-1.54 (m, 2H).

### Example 6

### Synthesis of rac-(1S*,2S*)—N-(5-((4-((1H-pyrazol-1-yl)methyl)benzyl)oxy)pyridazin-3-yl)-2-(1H-pyrazol-4-yl)cyclopropane-1-carboxamide (I-6)

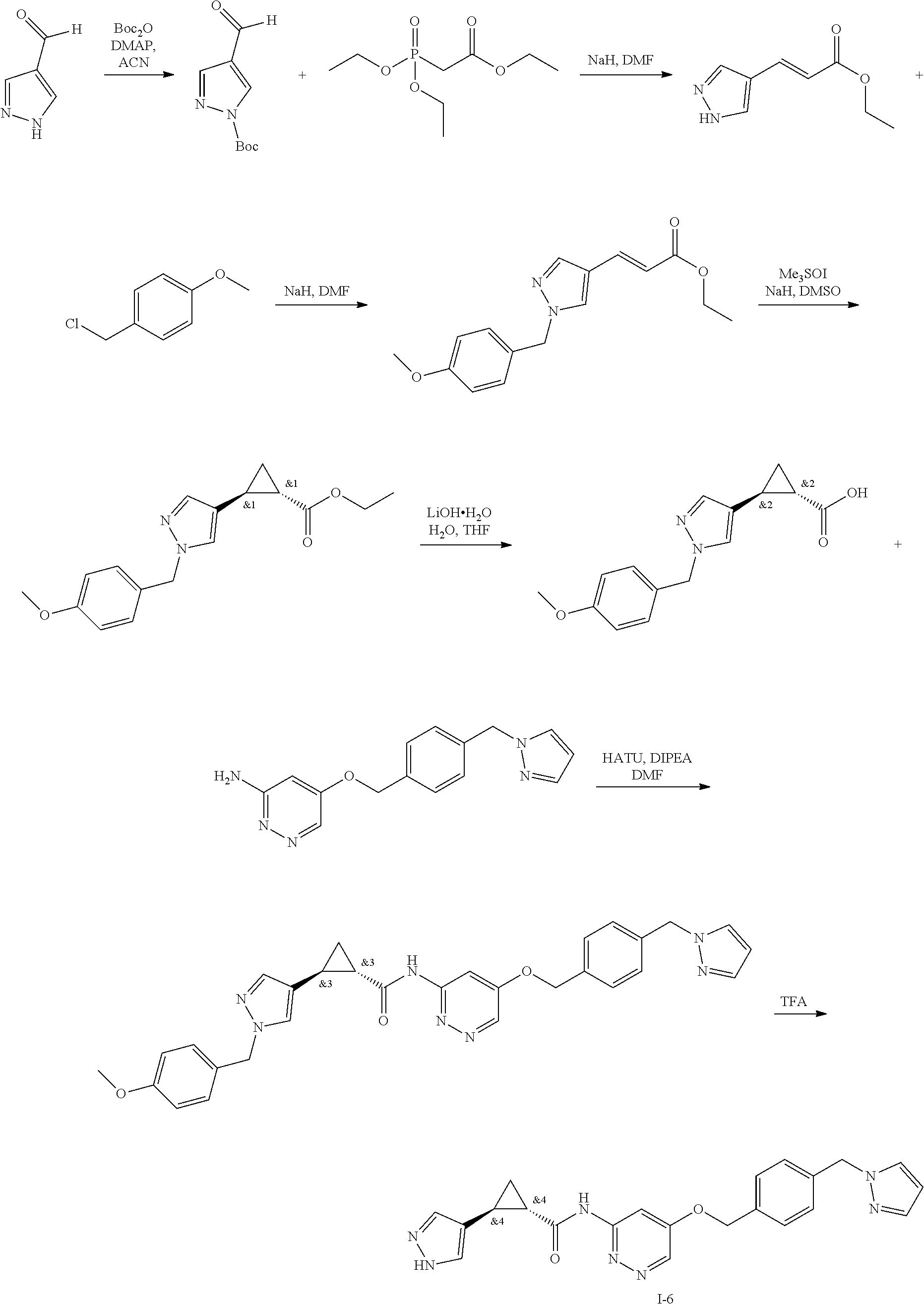

Synthesis of tert-butyl 4-formyl-1H-pyrazole-1-carboxylate. Di-tert-butyl dicarbonate (4.54 g, 20.81 mmol) was added to a solution of 1H-pyrazole-4-carbaldehyde (2.0 g, 20.81 mmol) in MeCN (21 ml) and the mixture was stirred at room temperature for 18 h. The reaction mixture was concentrated in vacuo to give an oil, which was diluted with water (20 mL) and extracted with EtOAc (3×20 mL). The organic phases were combined, washed with HCl (0.5M aq., 20 mL), brine (20 mL), passed through a phase separator cartridge and concentrated in vacuo to give the title compound (4.04 g, 98%) as a pale yellow solid. ¹H NMR (400 MHz, DMSO) δ 9.93 (s, 1H), 9.04 (s, 1H), 8.22 (s, 1H), 1.61 (s, 9H).

Synthesis of Ethyl (E)-3-(1H-pyrazol-4-yl)acrylate. A solution of triethylphosphonoacetate (4.49 mL, 1.03 mmol) in THF (40 mL) was added dropwise to an ice cooled suspension of NaH (60% in mineral oil, 1.24 g, 20.59 mmol) in THF (20 mL). The mixture was stirred for 30 min and a solution of 2-chloro-pyridine-3-carbaldehyde (4.04 g, 20.59 mmol) in THF (80 mL) was added dropwise. The mixture was allowed to warm to room temperature and stirred for 18 h. Reaction was quenched with HCl (2M, aq., 80 mL) and extracted with EtOAc (3×50 mL). The aqueous phase was cooled in ice, basified using sodium hydroxide (2M, aq.) and extracted with EtOAc (3×50 mL). The organic phase was passed through a phase separator cartridge and concentrated in vacuo to give a residue. Purification by silica gel column chromatography, eluting with a gradient of 0-100% ethyl acetate/hexane gave the title compound (1.12 g, 31%) as white solid. ¹H NMR (400 MHz, DMSO) δ 10.62 (brs, 1H), 8.08 (s, 2H), 7.57 (d, J=15.9 Hz, 1H), 6.35 (d, J=15.9 Hz, 1H), 4.14 (q, J=7.1 Hz, 2H), 1.24 (t, J=7.1 Hz, 3H).

Synthesis of ethyl (E)-3-(1-(4-methoxybenzyl)-1H-pyrazol-4-yl)acrylate. Ethyl (E)-3-(1H-pyrazol-4-yl)acrylate (1.2 g, 5.92 mmol) solution in DMF (10 mL) was added dropwise to a suspension of sodium hydride (60% mineral oil, 0.47 g, 11.84 mmol) in DMF (10 mL) and stirred at room temperature for 5 min. A solution of 4-methoxybenzyl chloride (0.84 mL, 6.22 mmol) in DMF (10 mL) was added dropwise and the mixture was stirred at room temperature for 18 h. The mixture was diluted with water (10 mL) and extracted with EtOAc (3×20 mL). The organic phases were combined and washed with brine (20 mL), passed through a phase separator cartridge and concentrated in vacuo to give a white solid. Purification by silica gel column chromatography, eluting with a gradient of 0-50% ethyl acetate in hexane gave the title compound (1.35 g, 80%) as white solid. ESI-MS (M+H)+: 287, ¹H NMR (400 MHz, DMSO) δ 8.20 (s, 1H), 7.91 (s, 1H), 7.52 (d, J=14.9 Hz, 1H), 7.23 (d, J=8.7 Hz, 2H), 6.91 (d, J=8.7 Hz, 2H), 6.30 (d, J=16.2 Hz, 1H), 5.24 (s, 2H), 4.14 (q, J=7.1 Hz, 2H), 3.33 (s, 3H), 1.23 (t, J=7.1 Hz, 3H).

Synthesis of ethyl rac-(S*,2S*)-2-(1-(4-methoxybenzyl)-1H-pyrazol-4-yl)cyclopropane-1-carboxylate. Sodium hydride (60% in mineral oil, 0.17 g, 4.37 mmol) was added portion-wise to a suspension of trimethylsulfoxonium iodide (1.15 g, 5.24 mmol) in DMSO (4.5 mL) at room temperature. After 1 hour, a solution of ethyl (E)-3-(1-(4-methoxybenzyl)-1H-pyrazol-4-yl)acrylate (0.5 g, 1.75 mmol) in DMSO (4.5 mL) was added dropwise and the resulting mixture was stirred for 18 h. Ammonium chloride (aq. sat., 2.55 mL) was added and the mixture was purified using C18 reverse phase chromatography eluting with 5-95% MeCN/water (10 mMol NH₄HCO₃) to give the title compound (87 mg, 16%) as yellow solid. ESI-MS (M+H)+: 301, ¹H NMR (400 MHz, DMSO) δ 7.65 (s, 1H), 7.31 (s, 1H), 7.18 (d, J=8.4 Hz, 2H), 6.89 (d, J=8.4 Hz, 2H), 5.14 (s, 2H), 4.08 (q, J=7.1 Hz, 2H), 3.73 (s, 3H), 2.25-2.18 (m, 1H), 1.77-1.70 (m, 1H), 1.37-1.31 (m, 1H), 1.23-1.15 (m, 4H).

Synthesis of rac-(1S*,2S*)-2-(1-(4-methoxybenzyl)-1H-pyrazol-4yl)cyclopropane-1-carboxylic acid. Ethyl rac-(1S*,2S*)-2-(1-(4-methoxybenzyl)-1H-pyrazol-4-yl)cyclopropane-1-carboxylate (129 mg, 0.43 mmol) in THF (0.43 mL) was added to an aqueous solution of lithium hydroxide (20 mg, 0.47 mmol) and stirred at room temperature for 18 h. The reaction mixture was acidified using HCl (1M aq., 1 mL) and extracted with EtOAc (3×5 mL). The organic phase was passed through a phase separator cartridge and concentrated in vacuo to give the title compound (108 mg, 92%) as a clear oil. ESI-MS (M+H)+: 273, ¹H NMR (400 MHz, DMSO) δ 8.20 (s, 1H), 7.91 (s, 1H), 7.52 (d, J=14.9 Hz, 1H), 7.23 (d, J=8.7 Hz, 2H), 6.91 (d, J=8.7 Hz, 2H), 6.30 (d, J=16.2 Hz, 1H), 5.24 (s, 2H), 4.14 (q, J=7.1 Hz, 2H), 3.33 (s, 3H), 1.23 (t, J=7.1 Hz, 3H).

Synthesis of rac-(1S*,2S*)—N-(5-((4-((1H-pyrazol-1-yl)methyl)benzyl)oxy)pyridazin-3-yl)-2-(1-(4-methoxybenzyl)-1H-pyrazol-4-yl)cyclopropane-1-carboxamide. Using a similar procedure to that used for rac-(1S*,2S*)—N-(5-((4-((1H-pyrazol-1-yl)methyl)benzyl)oxy)pyridazin-3-yl)-2-(5-chloropyridin-3-yl)cyclopropane-1-carboxamide, the title compound was synthesised from rac-(1S*,2S*)-2-(1-(4-methoxybenzyl)-1H-pyrazol-4-yl)cyclopropane-1-carboxylic acid and 5-((4-((1H-pyrazol-1-yl)methyl)benzyl)oxy)pyridazin-3-amine (72 mg, 39%). ESI-MS (M+H)+: 536, ¹H NMR (400 MHz, DMSO) δ 11.35 (s, 1H), 8.80 (d, J=2.8 Hz, 1H), 8.07-8.05 (m, 1H), 7.88 (d, J=2.0 Hz, 1H), 7.69 (s, 1H), 7.52-7.47 (m, 3H), 7.38 (s, 1H), 7.31-7.23 (m, 4H), 6.97-6.93 (m, 2H), 6.34-6.32 (m, 1H), 5.40 (s, 2H), 5.27 (s, 2H), 5.21 (s, 2H), 3.78 (s, 3H), 2.30-2.21 (m, 2H), 1.34-1.24 (m, 2H).

Synthesis of rac-(1S*,2S*)—N-(5-((4-((1H-pyrazol-1-yl)methyl)benzyl)oxy)pyridazin-3-yl)-2-(JH-pyrazol-4-yl)cyclopropane-1-carboxamide. rac-(1S*,2S*)—N-(5-((4-((1H-pyrazol-1-yl)methyl)benzyl)oxy)pyridazin-3-yl)-2-(1-(4-methoxybenzyl)-1H-pyrazol-4-yl)cyclopropane-1-carboxamide (30 mg, 0.11 mmol) was dissolved in TFA (0.11 mL) and stirred for 18 h. Anisole was added (0.006 mL, 0.11 mmol) and the mixture was heated to 50° C. for 2 h then to 100° C. for 12 h. The mixture was cooled then concentrated under a stream of N₂. The residue was dissolved in DCM (5 mL) then washed with NaHCO₃ solution (sat. aq., 2×5 mL). The organics were passed through a phase separator column then concentrated under a stream of N₂. The residue was purified by reversed-phase preparative HPLC to give the title compound (6 mg, 13%) as a formic acid salt. ESI-MS (M+H)+: 416.2, ¹H NMR (400 MHz, DMSO) δ 12.67-12.63 (m, 1H), 11.34 (s, 1H), 8.78 (d, J=2.5 Hz, 1H), 8.04 (d, J=2.5 Hz, 1H), 7.85 (d, J=1.8 Hz, 1H), 7.50-7.45 (m, 3H), 7.27 (d, J=8.3 Hz, 2H), 6.31-6.29 (m, 1H), 5.38 (s, 2H), 5.24 (s, 2H), 2.31-2.19 (m, 2H), 1.46-1.39 (m, 1H), 1.32-1.25 (m, 1H).

### Example 7

### Synthesis of N-(5-((4-((1H-pyrazol-1-yl)methyl)benzyl)oxy)pyridazin-3-yl)-2′-oxospiro[cyclopropane-1,3′-indoline]-2-carboxamide (I-7)

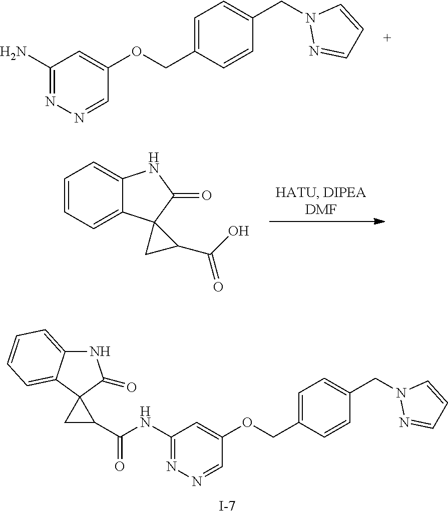

A solution of 2′-oxospiro[cyclopropane-1,3′-indoline]-2-carboxylic acid (54 mg, 0.27 mmol), HATU (100 mg, 0.27 mmol) and DIPEA (0.06 mL, 0.36 mmol) in DMF (2 mL) was stirred at room temperature for 5 min 5-((4-((1H-pyrazol-1-yl)methyl)benzyl)oxy)pyridazin-3-amine (50 mg, 0.18 mmol) was then added and the mixture was stirred at room temperature for 16 h. The reaction mixture was diluted with DCM (25 mL) and washed with an aqueous solution of sodium carbonate (10%, 25 mL). The organics were passed through a hydrophobic frit and concentrated in vacuo. The residue was purified by preparative HPLC to give the title compound (27 mg, 32%). ESI-MS (M+H)+: 467, H NMR (400 MHz, DMSO) δ 11.44 (s, 1H), 10.74 (s, 1H), 8.77 (d, J=2.8 Hz, 1H), 7.97 (d, J=2.3 Hz, 1H), 7.85 (d, J=1.5 Hz, 1H), 7.48 (d, J=8.1 Hz, 3H), 7.27 (d, J=8.1 Hz, 2H), 7.23-7.16 (m, 2H), 6.95-6.86 (m, 2H), 6.32-6.29 (m, 1H), 5.38 (s, 2H), 5.27 (s, 2H), 3.04 -2.98 (m, 1H), 2.17-2.12 (m, 1H), 1.88-1.83 (m, 1H).

### Example 8

### Synthesis of Synthesis of rac-(1S*,2S*)—N-(5-((4-((1H-pyrazol-1-yl)methyl)benzyl)oxy)pyridazin-3-yl)-2-(2-aminopyridin-3-yl)cyclopropane-1-carboxamide (I-8)

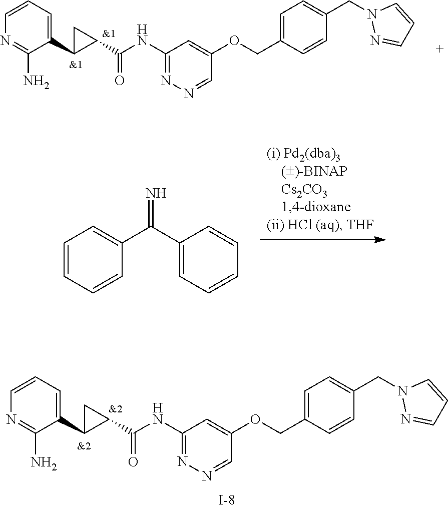

A mixture of rac-(1S*,2S*)—N-(5-((4-((1H-pyrazol-1-yl)methyl)benzyl)oxy)pyridazin-3-yl)-2-(2-chloropyridin-3-yl)cyclopropane-1-carboxamide (0.12 g, 0.26 mmol), Pd₂(dba)₃ (0.018 g, 0.020 mmol), (±)-BINAP (0.024 g, 0.039 mmol) and Cs₂CO₃ (0.25 g, 0.78 mmol) in 1,4-dioxane (2.2 mL) was degassed with N₂ then benzophenone imine (0.087 mL, 0.52 mmol) was added and the mixture was stirred at 105° C. under a N₂ atmosphere for 16 h. Further Pd₂(dba)₃ (0.018 g, 0.020 mmol), (±)-BINAP (0.024 g, 0.039 mmol) and Cs₂CO₃ (0.25 g, 0.78 mmol) were added and the mixture was degassed with N₂. Further benzophenone imine (0.087 mL, 0.52 mmol) was added and the mixture was stirred at 105° C. for 18 h. The mixture was diluted with DCM, filtered through Celite® and concentrated in vacuo. The residue was dissolved in THF (6.0 mL) and HCl (2.0 M aq., 0.72 mL) was added. The mixture was stirred at room temperature for 18 h then concentrated in vacuo. The residue was triturated with Et₂O and DCM and the solid was collected by filtration and dried in vacuo. The residue was purified by preparative HPLC to give the title compound (0.0070 g, 6%) as a pale yellow solid. ESI-MS (M+H): +442.2, ¹H NMR (400 MHz, CDCl₃) δ 10.72 (s, 1H), 8.54 (d, J=2.8 Hz, 1H), 8.22 (d, J=2.6 Hz, 1H), 7.99 (dd, J=1.4, 4.8 Hz, 1H), 7.56 (d, J=1.4 Hz, 1H), 7.43-7.39 (m, 3H), 7.35 (d, J=7.5 Hz, 1H), 7.25 (d, J=8.8 Hz, partially obscured by CHCl₃ peak, 2H), 6.63 (dd, J=5.0, 7.4 Hz, 1H), 6.30 (dd, J=2.1, 2.1 Hz, 1H), 5.35 (s, 2H), 5.15 (s, 2H), 4.63 (s, 2H), 2.49-2.35 (m, 2H), 1.72-1.66 (m, 1H), 1.35-1.29 (m, 1H).

### Example 9

### Synthesis of rac-(1S*,2S*)—N-(1-(4-((1H-pyrazol-1-yl)methyl)benzyl)-1H-imidazol-4yl)-2-(3-chlorophenyl)cyclopropane-1-carboxamide (1-9)

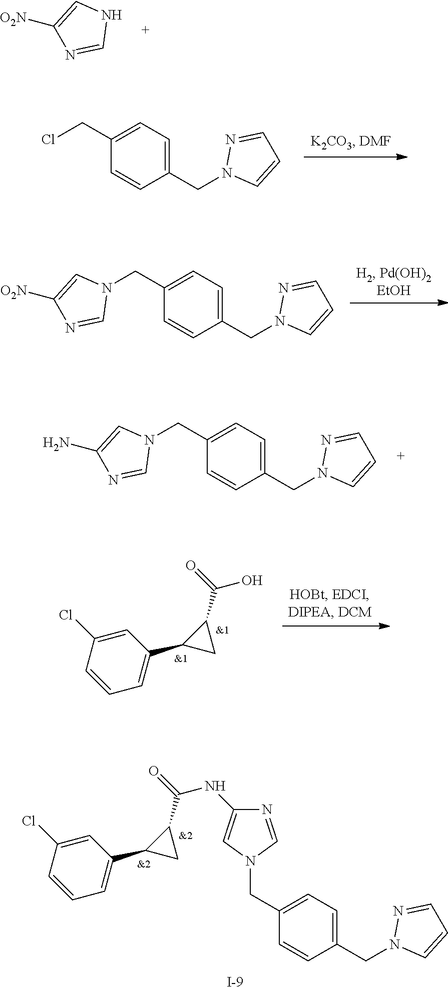

Synthesis of 1-(4-((4-nitro-1H-imidazol-1-yl)methyl)benzyl)-1H-pyrazole. A solution of 1-(4-(chloromethyl)benzyl)-1H-pyrazole (1.93 mmol, 400 mg) in DMF (1.5 mL) was added to a suspension of 4-nitro-imidazole (1.93 mmol, 218 mg) and K₂CO₃ (2.89 mmol, 400 mg) in DMF (2 mL). The mixture was stirred at room temperature overnight then concentrated in vacuo. The residue was suspended in water then filtered. The filtrant was dried in vacuo to give the title compound (531 mg, 97%). NMR analysis show this to be a 10:1 mixture of the desired: undesired regioisomer. The product was used without further purification. ESI-MS [M+H+]: 284.1

Synthesis of 1-(4-((1H-pyrazol-1-yl)methyl)benzyl)-1H-imidazol-4-amine. A solution of 1-(4-((4-nitro-1H-imidazol-1-yl)methyl)benzyl)-1H-pyrazole (0.56 mmol, 160 mg) in EtOH (4 mL) containing 20% palladium hydroxide on carbon (30 mg) was stirred under an atmosphere of H₂ overnight. The mixture was filtered through Celite® then concentrated in vacuo to give the title compound as a brown solid which was used without further purification. ESI-MS [M+H+]: 254.2

Synthesis of rac-(1S*,2S*)—N-(1-(4-((1H-pyrazol-1-yl)methyl)benzyl)-1H-imidazol-4-yl)-2-(3-chlorophenyl)cyclopropane-1-carboxamide. EDCI·HCl (0.18 mmol, 35 mg) and HOBt (0.17 mmol, 23 mg) were added to a solution of rac-(1S*,2S*)-2-(3-chlorophenyl)cyclopropane-carboxylic acid (0.16 mmol, 31 mg) in DCM (1 mL). The mixture was stirred for 5 minutes then a solution of 1-(4-((1H-pyrazol-1-yl)methyl)benzyl)-1H-imidazol-4-amine (0.16 mmol, 40 mg) and DIPEA (0.64 mmol, 0.11 mL) in DCM (1 mL) was added. The mixture was stirred for 2 hours, diluted with DCM then washed with NaHCO₃ (sat. aq.). The organics were passed through a phase separator cartridge then concentrated in vacuo. The residue was purified by reversed phase preparative HPLC to give the title compound (35 mg, 42%). ESI-MS [M+H+]: 432.3 ¹H NMR (400 MHz, DMSO) δ 10.55 (s, 1H), 7.81 (d, J=2.3 Hz, 1H), 7.56 (d, J=1.5 Hz, 1H), 7.45 (d, J=1.5 Hz, 1H), 7.31 (dd, J=7.7, 7.7 Hz, 1H), 7.25-7.17 (m, 6H), 7.15-7.11 (m, 2H), 6.26 (dd, J=2.1, 2.1 Hz, 1H), 5.32 (s, 2H), 5.11 (s, 2H), 2.35-2.28 (m, 1H), 2.19-2.14 (m, 1H), 1.43-1.29 (m, 2H).

### Example 10

### Synthesis of rac-(1S*,2S*)—N-(1-(4-((1H-pyrazol-1-yl)methyl)benzyl)-1H-pyrazol-4yl)-2-(3-chlorophenyl)cyclopropane-1-carboxamide (I-10)

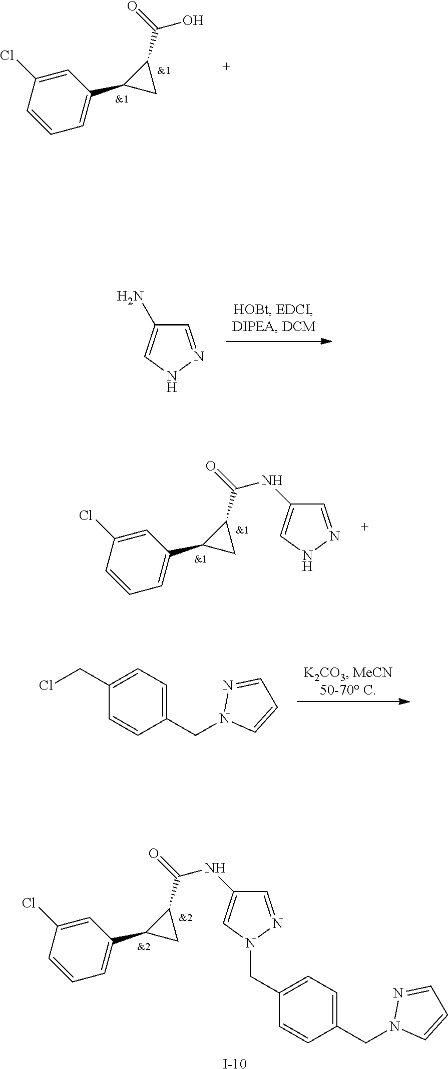

Synthesis of rac-(1S*,2S*)-2-(3-chlorophenyl)-N-(1H-pyrazol-4yl)cyclopropane-1-carboxamide. EDCI·HCl (1.98 mmol, 379 mg) and HOBt (1.89 mmol, 225 mg) were added to a solution of rac-(1S*,2S*)-2-(3-chlorophenyl)cyclopropane-1-carboxylic acid (1.80 mmol, 353 mg) in DCM (3 mL) cooled in an ice/water bath. The mixture was stirred for 5 minutes then a suspension of 4-amino-pyrazole (1.80 mmol, 150 mg) and DIPEA (5.4 mmol, 0.94 mL) in DCM (4 mL) was added. The mixture was then stirred overnight, allowing the temperature to rise to room temperature. The mixture was diluted with HCl (2M aq.) then extracted with DCM. The combined organics were washed with NaHCO₃ (sat. aq.) at which point a precipitate was observed. This was collected by filtration then washed (water then MeOH) and dried in vacuo to give the title compound as a lilac solid (152 mg, 31%). ESI-MS [M+H+]: 262.1

Synthesis of rac-(1S*,2S*)—N-(1-(4-((1H-pyrazol-1-yl)methyl)benzyl)-1H-pyrazol-4-yl)-2-(3-chlorophenyl)cyclopropane-1-carboxamide. A mixture of rac-(1S*,2*)-2-(3-chlorophenyl)-N-(1H-pyrazol-4-yl)cyclopropane-1-carboxamide (0.19 mmol, 50 mg) and K₂CO₃ (0.29 mmol, 39 mg) in MeCN (0.8 mL) was stirred for 20 min at room temperature. 1-(4-(chloromethyl)benzyl)-1H-pyrazole (0.20 mmol, 41 mg) was added and the mixture was stirred at 50° C. overnight. The temperature was then raised to 70° C. and stirring was continued for a further 16 hr. The mixture was diluted with water and EtOAc. The organics were separated, washed with brine, passed through a phase separator cartridge then concentrated in vacuo. The residue was purified by reversed phase preparative HPLC to give the title compound (35 mg, 42%). ESI-MS [M+H+]: 432.3 ¹H NMR (400 MHz, DMSO) δ 10.25 (s, 1H), 7.94 (s, 1H), 7.80 (d, J=1.8 Hz, 1H), 7.45-7.41 (m, 2H), 7.34-7.24 (m, 3H), 7.18-7.17 (m, 5H), 6.26 (t, J=2.0 Hz, 1H), 5.31 (s, 2H), 5.24 (s, 2H), 2.37-2.31 (m, 1H), 2.01-1.95 (m, 1H), 1.48-1.34 (m, 2H).

### Example 11

### Synthesis of rac-(1S*,2S*)-2-(3-chlorophenyl)-N-(5-((2-(4-methyl-1H-pyrazol-1-yl)pyridin-4-yl)methoxy)pyridazin-3-yl)cyclopropanecarboxamide (I-1)

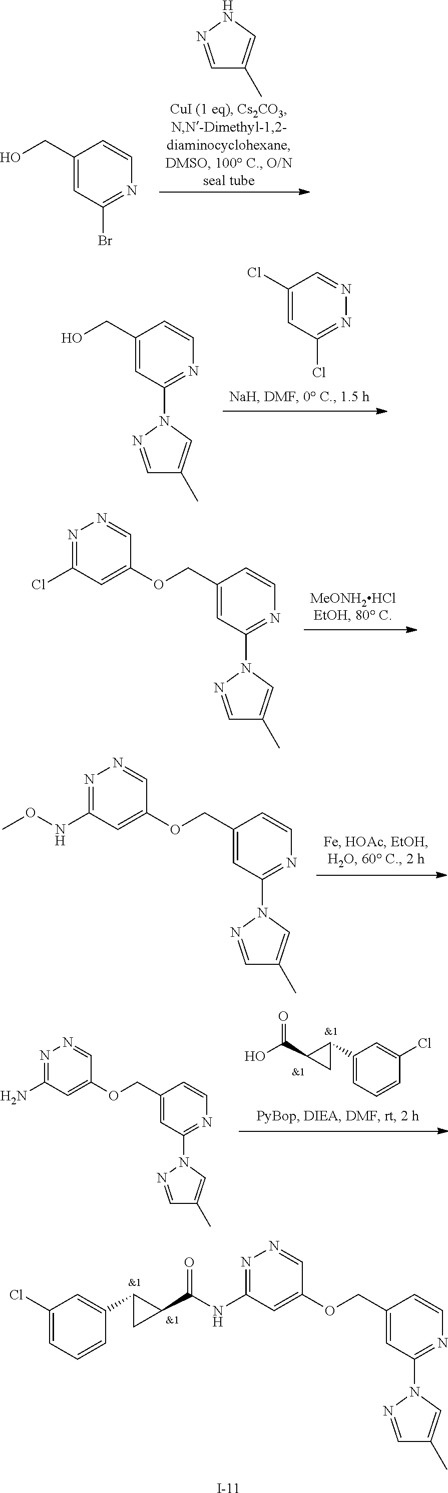

Synthesis of (2-(4-methyl-1H-pyrazol-1-yl)pyridin-4-yl)methanol A 10 mL of tube was charged with (2-bromo-pyridin-4-yl)-methanol (500 mg, 2.6 mmol), 4-methyl-1H-pyrazole (320 mg, 3.9 mmol), cesium carbonate (1.7 g, 5.2 mmol), cuprous iodide (490 mg, 2.6 mmol) and N,N′-dimethyl-cyclohexane-1,2-diamine (37 mg, 0.26 mmol) in dimethylsulphoxide (5 mL) was bubbled with a stream of nitrogen for 5 min. The tube was sealed and heated to 100° C. overnight. The reaction mixture was diluted with ethyl acetate and filtered through a pad of Celite. The filtrate was washed with water and extracted with ethyl acetate. The combined organic layers were washed with brine, dried over anhydrous sodium sulphate then concentrated in vacuo to give the crude product which was purified by silica gel (Petroleum ether/Ethyl acetate=10/1 to 2/1) to afford (2-(4-methyl-1H-pyrazol-1-yl)pyridin-4-yl)methanol (350 mg, 70%) as a yellow solid. LCMS: [M+H]⁺ 190.1.

Synthesis of 3-chloro-5-((2-(4-methyl-1H-pyrazol-1-yl)pyridin-4-yl)methoxy)pyridazine. To a solution of (2-(4-methyl-1H-pyrazol-1-yl)pyridin-4-yl)methanol (500 mg, 2.6 mmol) in N,N-dimethylformamide (14 mL) was added sodium hydride (125 mg, 5.2 mmol) at 0° C. under N₂. After stirring for 30 min, 3,5-dichloro-pyridazine (587 mg, 3.9 mmol) was added to the mixture. The resulting mixture was stirred at 0° C. for 1.5 hours and quenched with water at 0° C. and extracted with ethyl acetate. The organic phases were washed with brine, dried over anhydrous sodium sulphate and concentrated in vacuo. The residue was purified by silica gel (Petroleum ether /Ethyl acetate=5/1 to 2/1) to give 3-chloro-5-((2-(4-methyl-1H-pyrazol-1-yl)pyridin-4-yl)methoxy)pyridazine (500 mg, 64%) as a yellow solid. LCMS: [M+H]⁺ 302.1.

Synthesis of O-methyl-N-(5-((2-(4-methyl-1H-pyrazol-1-yl)pyridin-4-yl)methoxy)pyridazin-3-yl)hydroxylamine. A mixture of 3-chloro-5-((2-(4-methyl-1H-pyrazol-1-yl)pyridin-4-yl)methoxy)pyridazine (460 mg, 1.52 mmol) and O-methyl-hydroxylamine hydrochloride (1.9 g, 22.85 mmol) in ethanol (20 mL) was stirred at 80° C. overnight. Water (20 mL) and ethyl acetate (25 mL) were added. The organic phase was dried over anhydrous sodium sulphate and concentrated in vacuo to afford O-methyl-N-(5-((2-(4-methyl-1H-pyrazol-1-yl)pyridin-4-yl)methoxy)pyridazin-3-yl)hydroxylamine (510 mg crude) as an orange solid. LCMS: [M+H]⁺ 313.2.

Synthesis of 5-((2-(4-methyl-1H-pyrazol-1-yl)pyridin-4-yl)methoxy)pyridazin-3-amine. The mixture of O-methyl-N-(5-((2-(4-methyl-1H-pyrazol-1-yl)pyridin-4-yl)methoxy)pyridazin-3-yl)hydroxylamine (300 mg, 0.96 mmol), iron powder (537.6 mg, 9.6 mmol), acetic acid (0.8 mL), water (0.8 mL) and ethanol (15 mL) was stirred at 60° C. for 2 hours. The mixture was filtered and sodium carbonate (aqueous) was added into the filtrate to give pH 8. The mixture was extracted with ethyl acetate/methanol (3/1). The combined organic phase was concentrated in vacuo to afford 5-((2-(4-methyl-1H-pyrazol-1-yl)pyridin-4-yl)methoxy)pyridazin-3-amine (7, 173 mg, 48%) as yellow solid. LCMS: [M+H]⁺ 283.1.

Synthesis of rac-(1S*,2S*)-2-(3-chlorophenyl)-N-(5-((2-(4-methyl-1H-pyrazol-1-yl)pyridin-4-yl)methoxy)pyridazin-3-yl)cyclopropane-1-carboxamide. A mixture of rac-(1S*,2S*)-(3-chlorophenyl)cyclopropanecarboxylic acid (114 mg, 0.58 mmol), benzotriazole-1-yl-oxytripyrrolidinophosphonium hexafluorophosphate (PyBoP) (604 mg, 1.16 mmol) and ethyldiisopropylamine (223 mg, 1.73 mmol) in N,N-dimethylformamide (3 mL) was stirred at room temperature for 6 minutes. Then 5-((2-(4-methyl-1H-pyrazol-1-yl)pyridin-4-yl)methoxy)pyridazin-3-amine (163 mg, 0.58 mmol) was added. The mixture was stirred for 2 hours then water (15 mL) and ethyl acetate (25 mL) were added. The organic phase was dried over anhydrous sodium sulphate and concentrated in vacuo to give the crude product, which was purified by preparative HPLC to obtain rac-(1S*,2S*)-2-(3-chlorophenyl)-N-(5-((2-(4-methyl-1H-pyrazol-1-yl)pyridin-4-yl)methoxy)pyridazin-3-yl)cyclopropane-1-carboxamide (100 mg, 38%) as a white solid. LCMS: [M+H]⁺461.0 ¹H NMR (400 MHz, DMSO) δ 11.49 (s, 1H), 8.90 (d, J=2.4 Hz, 1H), 8.47 (d, J=5.2 Hz, 1H), 8.42 (s, 1H), 8.02 (d, J=2.8 Hz, 1H), 7.96 (s, 1H), 7.67 (s, 1H), 7.38 (d, J=4.4 Hz, 1H), 7.36-7.27 (m, 3H), 7.18 (d, J=7.6 Hz, 1H), 5.46 (s, 2H), 2.49-2.42 (m, 2H), 2.12 (s, 3H), 1.54-1.48 (m, 2H).

### Example 12

### Synthesis of rac-(1S*,2S*)—N-(5-((4-(1H-imidazol-1-yl)benzyl)oxy)pyridazin-3-yl)-2-(3-chlorophenyl)cyclopropane-1-carboxamide (1-12)

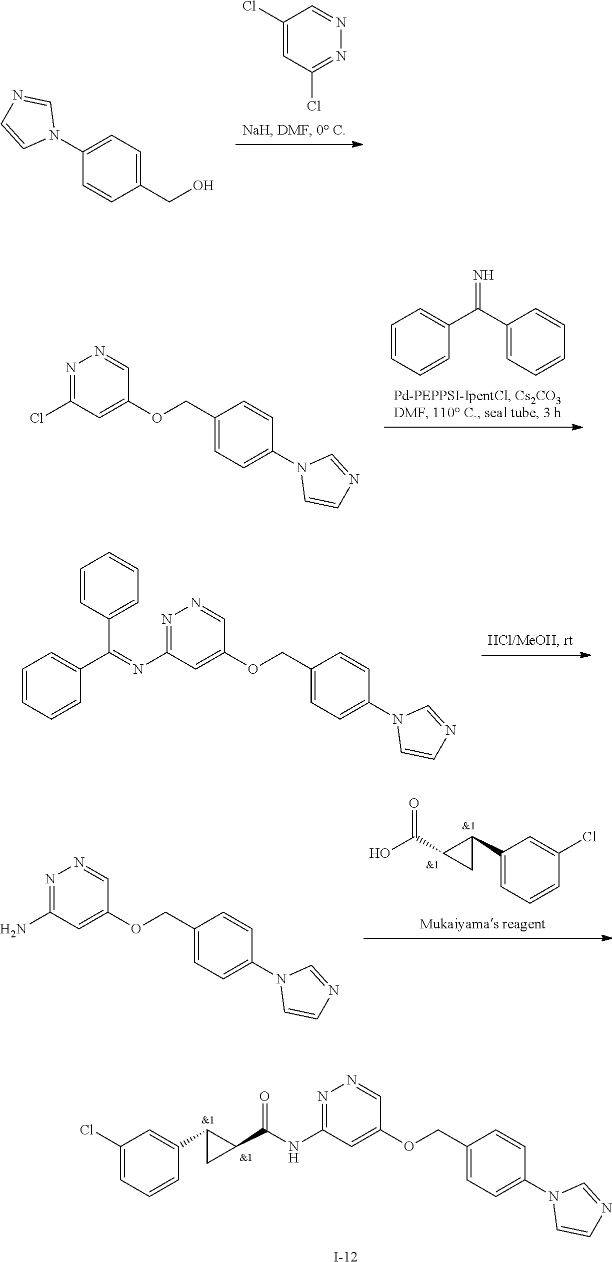

Synthesis of 5-((4-(1H-imidazol-1-yl)benzyl)oxy)-3-chloropyridazine. To a solution of (4-imidazol-1-yl-phenyl)-methanol (500 mg, 2.87 mmol) in N,N-dimethylformamide (10 mL) was added sodium hydride (138 mg, 3.44 mmol) at 0° C. After 30 min stirring at 0° C., 3,5-dichloro-pyridazine (425 mg, 2.87 mmol) was added at 0° C. The mixture was stirred at 0° C. for 2 hours. Water (8 mL) was added at 0° C. then the mixture was extracted with ethyl acetate (15 mL×4). The combined organic layers was concentrated in vacuo to give the crude product which was purified by silica gel (dichloromethane/methanol=20/1) to afford 5-((4-(1H-imidazol-1-yl)benzyl)oxy)-3-chloropyridazine (602 mg, 73%) as yellow solid. LCMS: [M+H]⁺ 287.1.

Synthesis of 5-((4-(1H-imidazol-1-yl)benzyl)oxy)-N-(diphenylmethylene)pyridazin-3-amine. To a mixture of 5-((4-(1H-imidazol-1-yl)benzyl)oxy)-3-chloropyridazine (400 mg, 1.4 mmol), diphenylmethanimine (506 mg, 2.8 mmol) and cesium carbonate (1.14 g, 3.5 mmol) in N,N-dimethylformamide (6 mL) was added Pd-PEPPSI-IPentCl (60 mg, 0.07 mmol) under a nitrogen atmosphere. The mixture was stirred at 53° C. for 50 min, then raised to 110° C. and stirred for another 3.5 hours. Water was added and the mixture extracted with ethyl acetate (20 mL). The organic phase was dried over anhydrous sodium sulphate and concentrated in vacuo to give crude product which was purified by silica gel (dichloromethane/methanol=15/1) to afford 5-((4-(1H-imidazol-1-yl)benzyl)oxy)-N-(diphenylmethylene)pyridazin-3-amine (510 mg, yield: 85%) as a red oil. LCMS: [M+H]⁺ 432.2

Synthesis of 5-((4-(1H-imidazol-1-yl)benzyl)oxy)pyridazin-3-amine. A mixture of 5-((4-(1H-imidazol-1-yl)benzyl)oxy)-N-(diphenylmethylene)pyridazin-3-amine (510 mg, 1.18 mmol) in hydrochloric acid (3M in methanol, 10 mL) was stirred at room temperature for 1 hour. The mixture was washed with petroleum ether (20 mL×3), then the mixture was adjusted to pH 8 with sat. sodium carbonate solution. After extraction with ethyl acetate (20 mL×5), the combined organic phases were concentrated in vacuo to give crude product which was washed with ethyl acetate to afford 5-((4-(1H-imidazol-1-yl)benzyl)oxy)pyridazin-3-amine (210 mg, 66%) as a white solid. LCMS: [M+H]⁺ 268.1.

Synthesis of rac-(1S*,2S*)—N-(5-((4-(H-imidazol-1-yl)benzyl)oxy)pyridazin-3-yl)-2-(3-chlorophenyl)cyclopropane-1-carboxamide. To a mixture of 5-((4-(1H-imidazol-1-yl)benzyl)oxy)pyridazin-3-amine (120 mg, 0.45 mmol) and rac-(1S*,2S*)-2-(3-chloro-phenyl)-cyclopropanecarboxylic acid (88 mg, 0.45 mmol) in dichloromethane (120 mL) was added Mukaiyama's reagent (92 mg, 0.36 mmol) followed by a solution of triethylamine (91 mg, 0.9 mmol) in dichloromethane (1 mL). The mixture became clear and was stirred at room temperature for 2 hours under a nitrogen atmosphere. The solvent was removed and potassium carbonate (124 mg, 0.9 mmol) in water (0.8 mL) was added. The mixture was stirred at 50° C. in acetonitrile (8 mL) overnight. Water (20 mL) and ethyl acetate (30 mL) were added. The organic phase was dried over anhydrous sodium sulphate and concentrated in vacuo to obtained crude product which was purified by silica gel (dichloromethane/methanol=15/1) then preparative HPLC to afford rac-(1S*,2S*)—N-(5-((4-(1H-imidazol-1-yl)benzyl)oxy)pyridazin-3-yl)-2-(3-chlorophenyl)cyclopropane-1-carboxamide (36 mg, 18%) as a colourless solid. LCMS: [M+H]⁺ 445.9 ¹H NMR (400 MHz, DMSO) δ 11.55 (s, 1H), 9.78 (s, 1H), 8.85 (d, J=2.4 Hz, 1H), 8.34 (s, 1H), 8.07 (d, J=2.0 Hz, 1H), 7.96 (s, 1H), 7.89 (d, J=8.4 Hz, 2H), 7.77 (d, J=8.4 Hz, 2H), 7.35-7.28 (m, 3H), 7.18 (d, J=8.0 Hz, 1H), 5.40 (s, 2H), 2.48-2.46 (m, 2H), 1.56-1.50 (m, 2H).

### Example 13

### Synthesis of rac-(1S*,2S*)—N-(5-((4-(1H-imidazol-2-yl)benzyl)oxy)pyridazin-3-yl)-2-(3-chlorophenyl)cyclopropane-1-carboxamide (I-13)

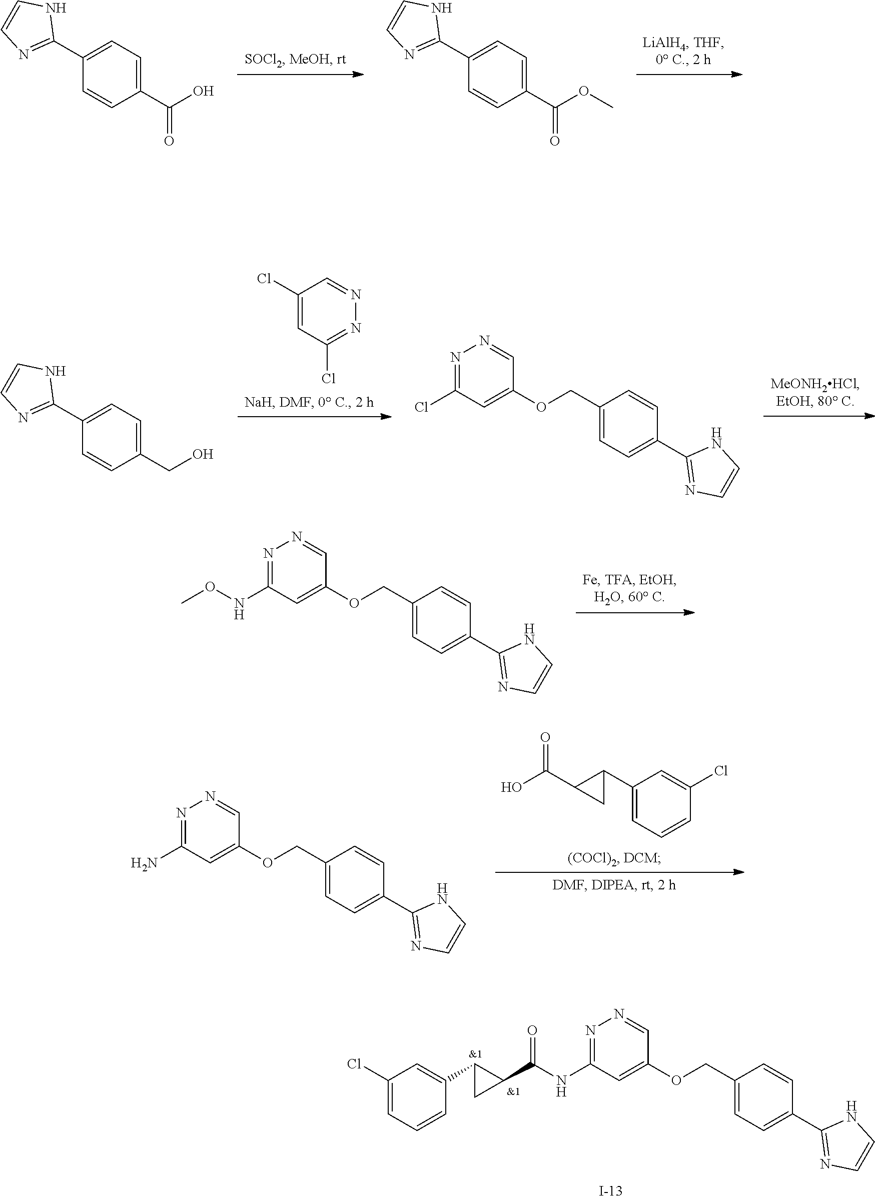

Synthesis of methyl 4-(1H-imidazol-2-yl)benzoate. To a solution of 4-(1H-imidazol-2-yl)benzoic acid (1 g, 5.3 mmol) in methanol (35 mL) was added thionyl chloride (6.3 g, 53 mmol) dropwise at 0° C. . After 30 min stirring at 0° C., the mixture was stirred at 30° C. overnight. The mixture was concentrated in vacuo to obtain a residue which was diluted by saturated sodium bicarbonate solution (25 mL), and extracted with dichloromethane (25×3 mL). The combined organic phases were dried, concentrated in vacuo and purified by silica gel column to afford methyl 4-(1H-imidazol-2-yl)benzoate (850 mg, 79%) as yellow solid. LCMS: [M+H]⁺ 203.1.

Synthesis of (4-(1H-imidazol-2-yl)phenyl)methanol. To a solution of methyl 4-(1H-imidazol-2-yl)benzoate (300 mg, 1.48 mmol) in tetrahydrofuran (5 mL) at 0° C., Lithium aluminum hydride (2.5 M in tetrahydrofuran, 1.77 mL, 4.44 mmol) was added. The mixture was stirred at 0° C. for 1 h. The reaction was quenched with sodium sulphate 10-hydrate and filtered through a pad of Celite, the filtrate was concentrated in vacuo and purified by silica gel column to afford (4-(1H-imidazol-2-yl)phenyl)methanol (176 mg, 68%) as colorless oil. LCMS: [M+H]⁺ 175.1.

Synthesis of 5-((4-(1H-imidazol-2-yl)benzyl)oxy)-3-chloropyridazine. To a solution of (4-(1H-imidazol-2-yl)phenyl)methanol (320 mg, 1.84 mmol) in N,N-dimethylformamide (5 mL) was added sodium hydride (184 mg, 4.6 mmol) at 0° C. After 30 min stirring at 0° C., 3,5-dichloro-pyridazine (329 mg, 2.21 mmol) was added. The mixture was stirred at 0° C. for 2 hours. The mixture was poured into water then exacted with ethyl acetate (30 mL×3). The combined organic phases were concentrated in vacuo and purified by silica gel column to afford 5-((4-(1H-imidazol-2-yl)benzyl)oxy)-3-chloropyridazine (350 mg, 67%) as white solid. LCMS: [M+H]⁺ 287.2.

Synthesis of N-(5-((4-(1H-imidazol-2-yl)benzyl)oxy)pyridazin-3-yl)-O— methylhydroxylamine. A mixture of 5-((4-(1H-imidazol-2-yl)benzyl)oxy)-3-chloropyridazine (230 mg, 0.80 mmol) and O-methyl-hydroxylamine hydrochloride (1.1 g, 12.06 mmol) in ethanol (10 mL) was stirred at 80° C. overnight. The mixture was adjust to pH 8 with saturated sodium bicarbonate, then extracted with dichloromethane (40×3 mL). The combined organic layers were concentrated in vacuo to afford N-(5-((4-(1H-imidazol-2-yl)benzyl)oxy)pyridazin-3-yl)-O-methylhydroxylamine (210 mg, 91%) as grey solid. LCMS: [M+H]⁺ 298.1.

Synthesis of 5-((4-(1H-imidazol-2-yl)benzyl)oxy)pyridazin-3-amine. A mixture of N-(5-((4-(1H-imidazol-2-yl)benzyl)oxy)pyridazin-3-yl)-O-methylhydroxylamine (230 mg, 0.774 mmol), iron powder (448.9 mg, 7.74 mmol), acetic acid (2 mL), water (5 mL) and ethanol (13 mL) was stirred at 60° C. for 4 hours. The mixture was filtered and the filtrate was basified to pH 9 with saturated sodium bicarbonate solution then extracted with dichloromethane (100 mL). The organic phase was concentrated in vacuo to afford 5-((4-(1H-imidazol-2-yl)benzyl)oxy)pyridazin-3-amine (140 mg, 68%) as yellow solid. LCMS: [M+H]⁺ 268.1.

Synthesis of rac-(1S*,2S*)—N-(5-((4-(H-imidazol-2yl)benzyl)oxy)pyridazin-3yl)-2-(3-chlorophenyl)cyclopropane-1-carboxamide. To a solution of rac-(1S*,2S*)-2-(3-chlorophenyl)cyclopropanecarboxylic acid (188 mg, 0.96 mmol) in dichloromethane (8 mL) was added oxalyl chloride (126.9 mg, 2.88 mmol) at 0° C. The mixture was stirred at 0° C. for 20 min. The solvent was concentrated in vacuo. The residue was diluted with N,N-dimethylformamide (6 mL) and 5-((4-(1H-imidazol-2-yl)benzyl)oxy)pyridazin-3-amine (256 mg, 0.96 mmol) and ethyldiisopropylamine (378 mg, 2.88 mmol) were added. The mixture was stirred at room temperature for 2 hours. Water was added and the mixture was extracted with ethyl acetate. The combined organic phases were washed with brine and dried over anhydrous sodium sulphate, then concentrated in vacuo. The crude product was purified by preparative TLC (dichloromethane /methanol=15/1) to afford rac-(1S*,2S*)—N-(5-((4-(1H-imidazol-2-yl)benzyl)oxy)pyridazin-3-yl)-2-(3-chlorophenyl)cyclopropane-1-carboxamide (18 mg, 4%) as a white solid. LCMS: [M+H]⁺ 446.0; ¹H NMR (400 MHz, DMSO) δ 12.57 (s, 1H), 11.38 (s, 1H), 8.81 (s, 1H), 8.05 (s, 1H), 7.98-7.96 (m, 2H), 7.56-7.54 (m, 2H), 7.35-7.27 (m, 3H), 7.19-7.16 (m, 3H), 5.28 (s, 2H), 2.47-2.43 (m, 2H), 1.54-1.49 (m, 2H).

### Example 14

### Synthesis of rac-(1S*,2S*)-2-(3-chlorophenyl)-N-(5-((4-((2-oxopyridin-1 (2H)-yl)methyl)benzyl)amino)pyridazin-3-yl)cyclopropane-1-carboxamide (I-14)

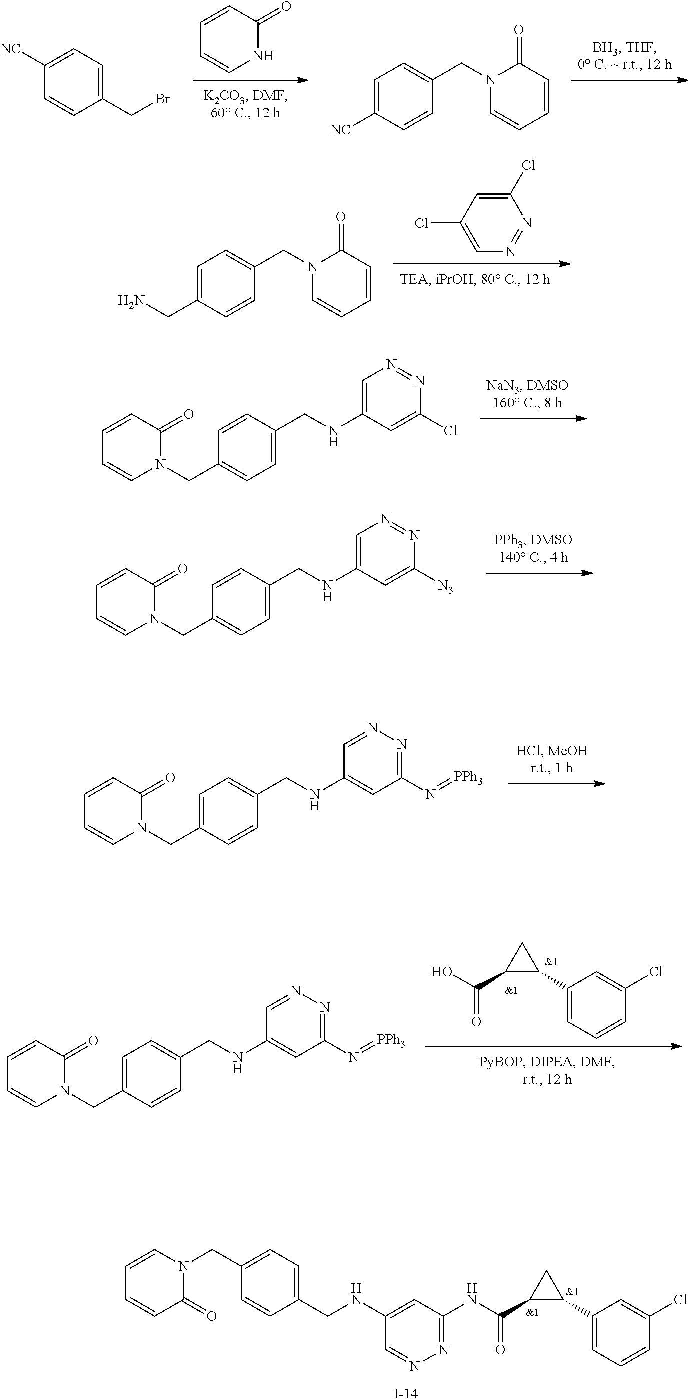

Synthesis of 4-((2-oxopyridin-1 (2H)-yl)methyl)benzonitrile. A mixture of 4-(bromomethyl)benzonitrile (10 g, 51 mmol), pyridin-2 (1H)-one (6.3 g, 66 mmol) and K₂CO₃ (14 g, 102 mmol) in DMF (60 mL) was stirred at 60° C. for 12 h. After the reaction was cooled to room temperature, H₂O (200 mL) was added and extracted with EtOAc (200 mL×3). The combined organic layers were dried over anhydrous Na₂SO₄, concentrated in vacuo to give crude, which was purified with silica gel column chromatography eluting with a gradient of 0-20% EtOAc in PE to give 4-((2-oxopyridin-1 (2H)-yl)methyl)benzonitrile (8.5 g, 79%) as a white solid. ESI-MS [M+H]⁺: 211.2.

Synthesis of 1-(4-(aminomethyl)benzyl)pyridin-2 (1H)-one. To a solution of 4-((2-oxopyridin-1 (2H)-yl)methyl)benzonitrile (8.5 g, 40.3 mmol) in THF (50 mL) was added BH₃-THF (100 mL, 100 mmol, 1 M solution in THF) slowly at 0° C. The resulting reaction was stirred at room temperature for 12 h. The reaction was cooled to 0° C., MeOH (50 mL) was added slowly and the reaction mixture was stirred at room temperature for 1 h, then concentrated in vacuo to give the crude, which was purified with silica gel column chromatography eluting with a gradient of 0-8% MeOH in DCM to give 1-(4-(aminomethyl)benzyl)pyridin-2 (1H)-one (6.8 g, 79%) as a white solid. ESI-MS [M+H]⁺: 214.2.

Synthesis of 1-(4-(((6-chloropyridazin-4-yl)amino)methyl)benzyl)pyridin-2 (1H)-one. A mixture of 1-(4-(aminomethyl)benzyl)pyridin-2 (1H)-one (6.8 g, 31.8 mmol), 3,5-dichloropyridazine (4.7 g, 31.8 mmol) and TEA (8 g, 79.5 mmol) in iPrOH (80 mL) was stirred at 80° C. for 12 h. The reaction was cooled to room temperature, concentrated in vacuo to give the crude, which was purified with silica gel column chromatography eluting with a gradient of 0-5% MeOH in DCM to give 1-(4-(((6-chloropyridazin-4-yl)amino)methyl)benzyl)pyridin-2 (1H)-one (8.8 g, 85%) as a yellow solid. ESI-MS [M+H]⁺: 327.2.

Synthesis of 1-(4-(((6-azidopyridazin-4-yl)amino)methyl)benzyl)pyridin-2(1H)-one. A mixture of 1-(4-(((6-chloropyridazin-4-yl)amino)methyl)benzyl)pyridin-2 (1H)-one (1 g, 3.1 mmol) and NaN₃ (598 mg, 9.2 mmol) in DMSO (15 mL) was stirred at 160° C. for 8 h. After the reaction was cooled to room temperature, H₂O (80 mL) was added and extracted with EtOAc (80 mL×3). The combined organic layers were washed with brine (80 mL), dried over anhydrous Na₂SO₄ then concentrated in vacuo to give crude, which was purified with silica gel column chromatography eluting with a gradient of 0-8% MeOH in DCM to give 1-(4-(((6-azidopyridazin-4-yl)amino)methyl)benzyl)pyridin-2 (1H)-one (520 mg, 50%) as a yellow solid. ESI-MS [M+H]⁺: 334.2.

Synthesis of 1-(4-(((6-((triphenyl-15-phosphaneylidene)amino)pyridazin-4-yl)amino)methyl)benzyl)pyridin-2(1H)-one. A mixture of 1-(4-(((6-azidopyridazin-4-yl)amino)methyl)benzyl)pyridin-2 (1H)-one (520 mg, 1.6 mmol) and PPh₃ (1.3 g, 5 mmol) in DMSO (10 mL) was stirred at 140° C. for 4 h. The reaction was cooled to room temperature, diluted with H₂O (30 mL) and extracted with EtOAc (50 mL×3). The combined organic layers were washed with brine (50 mL), dried over anhydrous Na₂SO₄, concentrated in vacuo to give 1-(4-(((6-((triphenyl-15-phosphaneylidene)amino)pyridazin-4-yl)amino)methyl)benzyl)pyridin-2 (1H)-one (1.4 g, crude) as a yellow solid, which was used without further purification. ESI-MS [M+H]⁺: 568.2.

Synthesis of 1-(4-(((6-aminopyridazin-4-yl)amino)methyl)benzyl)pyridin-2(1H)-one. To a solution of 1-(4-(((6-((triphenyl-15-phosphaneylidene)amino)pyridazin-4-yl)amino)methyl)benzyl)pyridin-2 (1H)-one (1.4 g, crude) in MeOH (20 mL) was added HCl (6 M aq. 5 mL). The resulting reaction was stirred at room temperature for 1 h. The reaction was concentrated in vacuo to remove MeOH. The residue was washed with NaHCO₃ (sat. aq., 60 mL), extracted with CHCl₃/iPrOH (3/1, 40 mL×4). The combined organic layers were washed with brine (50 mL), dried over anhydrous Na₂SO₄ and concentrated in vacuo to give the crude, which was purified with silica gel column chromatography eluting with a gradient of 0-15 MeOH (10% of NH₃H₂O as additive) in DCM to give 1-(4-(((6-aminopyridazin-4-yl)amino)methyl)benzyl)pyridin-2 (1H)-one (385 mg, 78% for 2 steps) as a yellow solid. ESI-MS [M+H]⁺: 308.2.

Synthesis of rac-(1S*,2S*)-2-(3-chlorophenyl)-N-(5-((4-((2-oxopyridin-1 (2H)-yl)methyl)benzyl)amino)pyridazin-3-yl)cyclopropane-1-carboxamide. To a solution of 1-(4-(((6-aminopyridazin-4-yl)amino)methyl)benzyl)pyridin-2 (1H)-one (100 mg, 0.33 mmol), rac-(1S*,2S*)-2-(3-chlorophenyl)cyclopropane-1-carboxylic acid (98 mg, 0.50 mmol) and PyBOP (343 mg, 0.66 mmol) in DMF (10 mL) was added DIPEA (206 mg, 1.6 mmol). The resulting reaction was stirred at room temperature for 12 nh then diluted with H₂O (20 mL) and extracted with EtOAc (30 mL×3). The combined organic layers were washed with brine (40 mL), dried over anhydrous Na₂SO₄, concentrated in vacuo to give the crude, which was purified with preparative TLC (eluent: DCM/MeOH=15/1) to give rac-(1S*,2S*)-2-(3-chlorophenyl)-N-(5-((4-((2-oxopyridin-1 (2H)-yl)methyl)benzyl)amino)pyridazin-3-yl)cyclopropane-1-carboxamide (43 mg, 27%) as a white solid (mixture of enantiomers). ESI-MS [M+H]⁺: 486.2. ¹H NMR (400 MHz, DMSO) δ 10.91 (s, 1H), 8.35 (d, J=2.6 Hz, 1H), 7.78 (dd, J=6.8, 1.7 Hz, 1H), 7.67 (s, 1H), 7.47-7.35 (m, 2H), 7.36-7.18 (m, 7H), 7.15 (d, J=7.6 Hz, 1H), 6.40 (d, J=9.1 Hz, 1H), 6.23 (td, J=6.7, 1.3 Hz, 1H), 5.07 (s, 2H), 4.30 (d, J=5.6 Hz, 2H), 2.45-2.25 (m, 2H), 1.52-1.35 (m, 2H).

### Examples 15 and 16

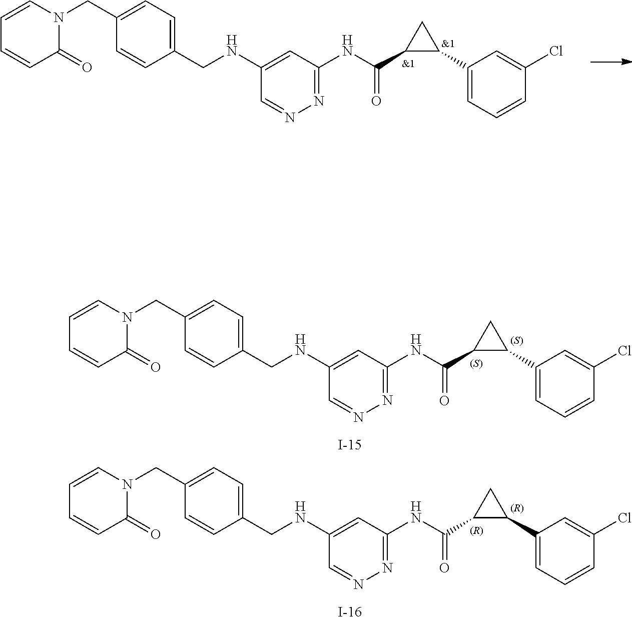

The mixture was separated using chiral column separation [CHIRALPAK OJ, 2.5 cm I.D×15 cm L MEOH/ACN=90/10 (V/V), 40 mL/min, 35° C.) to give two enantiomers: (1S,2S)-2-(3-chlorophenyl)-N-(5-((4-((2-oxopyridin-1 (2H)-yl)methyl)benzyl)amino)pyridazin-3-yl)cyclopropane-1-carboxamide and (1R,2R)-2-(3-chlorophenyl)-N-(5-((4-((2-oxopyridin-1 (2H)-yl)methyl)benzyl)amino)pyridazin-3-yl)cyclopropane-1-carboxamide.

First eluting isomer, (1S,2S)-2-(3-chlorophenyl)-N-(5-((4-((2-oxopyridin-1 (2H)-yl)methyl)benzyl)amino)pyridazin-3-yl)cyclopropane-1-carboxamide (I-15). ESI-MS [M+H]⁺: 486.2. ¹H NMR (400 MHz, DMSO) δ 10.91 (s, 1H), 8.35 (d, J=2.6 Hz, 1H), 7.78 (dd, J=6.8, 1.7 Hz, 1H), 7.67 (s, 1H), 7.48-7.37 (m, 2H), 7.37-7.22 (m, 7H), 7.15 (d, J=7.6 Hz, 1H), 6.40 (d, J=9.1 Hz, 1H), 6.23 (td, J=6.7, 1.3 Hz, 1H), 5.07 (s, 2H), 4.30 (d, J=5.6 Hz, 2H), 2.45-2.25 (m, 2H), 1.54-1.36 (m, 2H). RT=3.2 min, 99.0% e.e.

Second eluting isomer, (1R,2R)-2-(3-chlorophenyl)-N-(5-((4-((2-oxopyridin-1 (2H)-yl)methyl)benzyl)amino)pyridazin-3-yl)cyclopropane-1-carboxamide (I-16). ESI-MS [M+H]⁺: 486.2. ¹H NMR (400 MHz, DMSO) δ 10.91 (s, 1H), 8.35 (d, J=2.6 Hz, 1H), 7.78 (dd, J=6.8, 1.7 Hz, 1H), 7.67 (s, 1H), 7.48-7.37 (m, 2H), 7.37-7.22 (m, 7H), 7.15 (d, J=7.6 Hz, 1H), 6.40 (d, J=9.1 Hz, 1H), 6.23 (td, J=6.7, 1.3 Hz, 1H), 5.07 (s, 2H), 4.30 (d, J=5.6 Hz, 2H), 2.45-2.25 (m, 2H), 1.54-1.36 (m, 2H). RT=4.1 min, 98.9% e.e.

### Example 17

### Synthesis of rac-(1S*,2S*)—N-(5-((4-((5-chloro-2-oxopyridin-1 (2H)-yl)methyl benzyl amino)pyridazin-3-yl)-2-(3-chlorophenyl)cyclopropane-1-carboxamide (I-17)

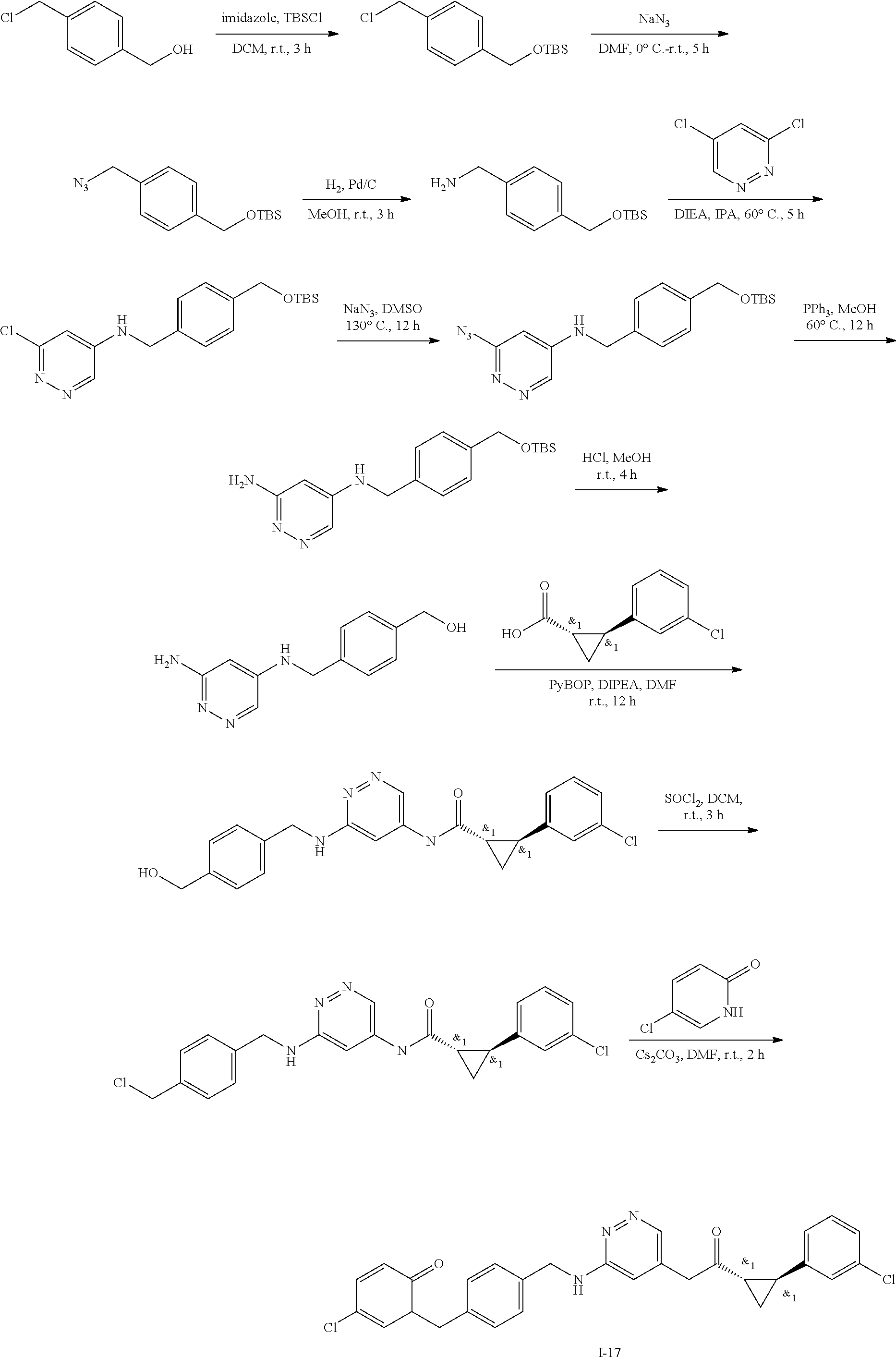

Synthesis of tert-butyl((4-(chloromethyl)benzyl)oxy)dimethylsilane. To a mixture of (4-(chloromethyl)phenyl)methanol (5.0 g, 32 mmol), imidazole (6.5 g, 96 mmol) in DCM (50 mL) was added TBSCl (7.2 g, 48 mmol) and the mixture was stirred at room temperature for 3 h. The reaction was quenched with water (100 mL) and extracted with DCM (100 mL×3). The combined organic layers were washed with brine (100 mL), dried over Na₂SO₄ and concentrated in vacuo to give the crude, which was purified by column chromatography (eluent: EtOAc/PE=0-10%) to give tert-butyl((4-(chloromethyl)benzyl)oxy)dimethylsilane (7.0 g, 80%) as a yellow oil. ESI-MS [M+H]⁺: 271.1.

Synthesis of ((4-(azidomethyl)benzyl)oxy)(tert-butyl)dimethylsilane. To a mixture of tert-butyl((4-(chloromethyl)benzyl)oxy)dimethylsilane (3.0 g, 11.1 mmol) in DMF (10 mL) was added NaN₃ (1.4 g, 22.2 mmol) at 0° C. and the mixture was stirred at room temperature for 5 h. The reaction was poured into water (100 mL) and extracted with EtOAc (100 mL×3). The combined organic layers were washed with brine (100 mL), dried over Na₂SO₄ and concentrated to give the crude, which was purified by column chromatography (eluent: EtOAc/PE=0-10%) to give ((4-(azidomethyl)benzyl)oxy)(tert-butyl)dimethylsilane (2.4 g, 77%) as a white solid. ESI-MS [M+H]+: 278.2

Synthesis of (4-(((tert-butyldimethylsilyl)oxy)methyl)phenyl)methanamine. A mixture of ((4-(azidomethyl)benzyl)oxy)(tert-butyl)dimethylsilane (500 mg, 1.8 mmol) and Pd/C (100 mg) in MeOH (10 mL) was stirred at room temperature for 3 h under H₂. The reaction mixture was filtered through Celite® and the filter cake was washed with MeOH (50 mL). The filtrate was concentrated in vacuo to give (4-(((tert-butyldimethylsilyl)oxy)methyl)phenyl)methanamine (400 mg, 88%) as a brown oil. ESI-MS [M+H]⁺: 252.2.

Synthesis of N-(4-(((tert-butyldimethylsilyl)oxy)methyl)benzyl)-6-chloropyridazin-4-amine. A mixture of (4-(((tert-butyldimethylsilyl)oxy)methyl)phenyl)methanamine (400 mg, 1.6 mmol), 3,5-dichloropyridazine (238 mg, 1.6 mmol) and DIPEA (417 mg, 3.2 mmol) ) in IPA (10 mL) was stirred at 60° C. for 5 h. After cooling to room temperature, the reaction was poured into water (30 mL) and extracted with DCM (50 mL×3). The combined organic layers were washed with brine (30 mL), dried over Na₂SO₄ then concentrated in vacuo to give the crude, which was purified by column chromatography (eluent: EtOAc/PE=0-30%) to give N-(4-(((tert-butyldimethylsilyl)oxy)methyl)benzyl)-6-chloropyridazin-4-amine (300 mg, 52%) as a white solid. ESI-MS [M+H]⁺: 364.2.

Synthesis of 6-azido-N-(4-(((tert-butyldimethylsilyl)oxy)methyl)benzyl)pyridazin-4-amine. A mixture of N-(4-(((tert-butyldimethylsilyl)oxy)methyl)benzyl)-6-chloropyridazin-4-amine (1.2 g, 3.3 mmol) and NaN₃ (644 mg, 9.9 mmol) in DMSO (10 mL) was stirred at 130° C. for 12 h. After cooling to room temperature, the reaction was diluted with H₂O (40 mL), extracted with EtOAc (40 mL×3). The combined organic layers were washed with brine (40 mL), dried over anhydrous Na₂SO₄, concentrated in vacuo to give the crude, which was purified with silica gel column chromatography eluting with a gradient of 0-10% MeOH in DCM to give 6-azido-N-(4-(((tert-butyldimethylsilyl)oxy)methyl)benzyl)pyridazin-4-amine (700 mg, 57%) as a yellow solid. ESI-MS [M+H]⁺: 371.2

Synthesis of N5-(4-(((tert-butyldimethylsilyl)oxy)methyl)benzyl)pyridazine-3,5-diamine. A mixture of 6-azido-N-(4-(((tert-butyldimethylsilyl)oxy)methyl)benzyl)pyridazin-4-amine (700 mg, 1.9 mmol) and PPh₃ (1.5 g, 5.7 mmol) in MeOH (30 mL) was stirred at 60° C. for 12 h. The reaction was concentrated in vacuo to give the crude, which was purified with silica gel column chromatography eluting with a gradient 0-15% MeOH in DCM to give N₅-(4-(((tert-butyldimethylsilyl)oxy)methyl)benzyl)pyridazine-3,5-diamine (435 mg, 67%) as a yellow solid. ESI-MS [M+H]⁺: 345.1

Synthesis of (4-(((6-aminopyridazin-4-yl)amino)methyl)phenyl)methanol. To a solution N₅-(4-(((tert-butyldimethylsilyl)oxy)methyl)benzyl)pyridazine-3,5-diamine (435 mg, 1.3 mmol) in MeOH (20 mL) was added HCl (2 mL, 8 mmol, 4 M solution in MeOH). The resulting mixture was stirred at room temperature for 4 h. The reaction was concentrated in vacuo to give (4-(((6-aminopyridazin-4-yl)amino)methyl)phenyl)methanol (400 mg, crude) as yellow oil, which was used without further purification. ESI-MS [M+H]⁺: 231.1.

Synthesis of rac-(1S*,2S*)-2-(3-chlorophenyl)-N-(5-((4-(hydroxymethyl)benzyl)amino)pyridazin-3-yl)cyclopropane-1-carboxamide. To a solution of (4-(((6-aminopyridazin-4-yl)amino)methyl)phenyl)methanol (400 mg, crude) and rac-(1S*,2S*)-2-(3-chlorophenyl)cyclopropane-1-carboxylic acid (255 mg, 1.3 mmol), PyBOP (832 mg, 1.6 mmol) in DMF (20 mL) was added DIPEA (1 g, 7.8 mmol). The reaction solution was stirred at room temperature for 12 h. then diluted with H₂O (50 mL) and extracted with EtOAc (40 mL×3). The combined organic layers were washed with brine (50 mL), dried over anhydrous Na₂SO₄ and concentrated in vacuo to give the crude, which was purified by silica gel column chromatography eluting with a gradient of 0-10% MeOH in DCM to offer rac-(1S*,2S*)-2-(3-chlorophenyl)-N-(5-((4-(hydroxymethyl)benzyl)amino)pyridazin-3-yl)cyclopropane-1-carboxamide (115 mg, 22% for 2 steps) as a yellow solid. ESI-MS [M+H]⁺: 409.2.

Synthesis of rac-(1S*,2S*)—N-(5-((4-(chloromethyl)benzyl)amino)pyridazin-3yl)-2-(3-chlorophenyl)cyclopropane-1-carboxamide. To a solution of rac-(1S*,2S*)-2-(3-chlorophenyl)-N-(5-((4-(hydroxymethyl)benzyl)amino)pyridazin-3-yl)cyclopropane-1-carboxamide (115 mg, 0.28 mmol) in DCM (10 mL) was added SOCl₂ (1 mL). The resulting reaction solution was stirred at room temperature for 3 h. After completed, the reaction was concentrated in vacuo to give rac-(1S*,2S*)—N-(5-((4-(chloromethyl)benzyl)amino)pyridazin-3-yl)-2-(3-chlorophenyl)cyclopropane-1-carboxamide (130 mg, crude) as a yellow solid, which was used into next step without further purification. ESI-MS [M+H]⁺: 427.2.

Synthesis of rac-(1S*,2S*)—N-(5-((4-((5-chloro-2-oxopyridin-1 (2H)-yl)methyl)benzyl)amino)pyridazin-3-yl)-2-(3-chlorophenyl)cyclopropane-1-carboxamide. A mixture of rac-(1S*,2S*)—N-(5-((4-(chloromethyl)benzyl)amino)pyridazin-3-yl)-2-(3-chlorophenyl)cyclopropane-1-carboxamide (130 mg, crude), 5-chloropyridin-2 (1H)-one (54 mg, 0.42 mmol) and Cs₂CO₃ (365 mg, 1.1 mmol) in DMF (10 mL) was stirred at room temperature for 2 h. The reaction was diluted with H₂O (30 mL), extracted with EtOAc (40 mL×3). The combined organic layers were washed with brine (40 mL), dried over anhydrous Na₂SO₄, concentrated in vacuo to give the crude, which was purified with silica gel column chromatography eluting with a gradient of 0-10% MeOH in MeOH to give rac-(1S*,2S*)—N-(5-((4-((5-chloro-2-oxopyridin-1 (2H)-yl)methyl)benzyl)amino)pyridazin-3-yl)-2-(3-chlorophenyl)cyclopropane-1-carboxamide (23 mg, 16%) as a white solid. ESI-MS [M+H]⁺: 520.2. ¹H NMR (400 MHz, DMSO) δ 10.83 (s, 1H), 8.35 (d, J=2.6 Hz, 1H), 8.26 (s, 1H), 8.10 (d, J=2.8 Hz, 1H), 7.55 (t, J=5.9 Hz, 1H), 7.48 (dd, J=9.7, 2.9 Hz, 1H), 7.43 (s, 1H), 7.34-7.25 (m, 5H), 7.17-7.14 (m, 1H), 6.45 (d, J=9.7 Hz, 1H), 5.05 (s, 2H), 4.29 (d, J=5.7 Hz, 2H), 2.42-2.37 (m, 2H), 1.48-1.37 (m, 2H).

### Example 18

Synthesis of rac-(1S*,2S*)—N-(5-((4-((1H-pyrazol-1-yl)methyl)benzyl)oxy)pyridazin-3-yl)-2-(3-chlorophenyl)cyclopropane-1-carboxamide (I-18)

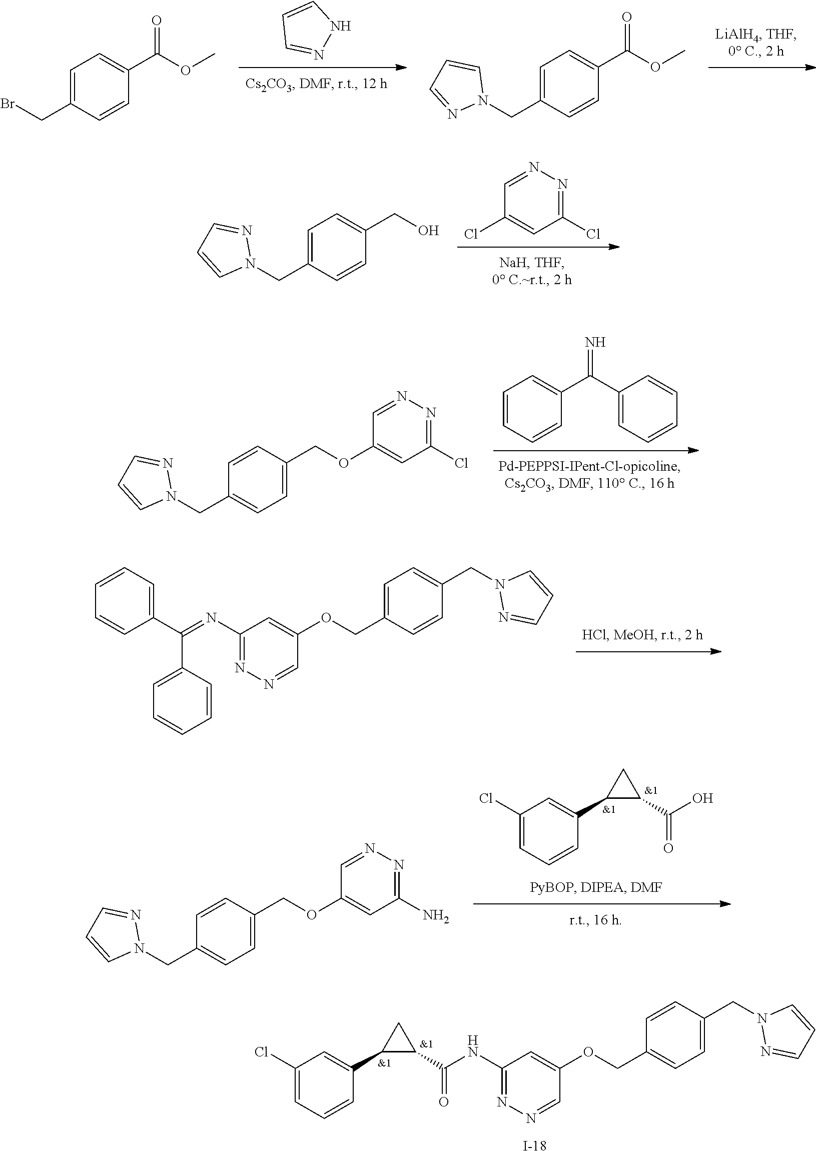

Synthesis of methyl 4-((1H-pyrazol-1-yl)methyl)benzoate. A mixture of methyl 4-(bromomethyl)benzoate (20 g, 87.3 mmol), 1H-pyrazole (10 g, 147.3 mmol) and Cs₂CO₃ (57 g, 174 mmol) in DMF (150 mL) was stirred at room temperature for 12 h. The reaction was diluted with H₂O (500 mL), extracted with EtOAc (300 mL×3). The combined organic layers were washed with brine (200 mL), dried over anhydrous Na₂SO₄ then concentrated in vacuo to give the crude, which was purified with silica gel column chromatography eluting with a gradient of 0-20% EtOAc in PE to give methyl 4-((1H-pyrazol-1-yl)methyl)benzoate (17.1 g, 91%) as a white solid. ESI-MS [M+H]⁺: 217.2.

Synthesis of (4-((1H-pyrazol-1-yl)methyl)phenyl)methanol. To a solution of methyl 4-((1H-pyrazol-1-yl)methyl)benzoate (17.1 g, 78.8 mmol) in THF (150 mL) was added LiAiH₄ (3.6 g, 94.6 mmol) slowly at 0° C. The resulting reaction was stirred at 0° C. for 2 h. The reaction was quenched with Na₂SO₄-10H₂O (50 g) carefully at 0° C. and stirred at this temperature for 1 h, then filtered through Celite®. The filter cake washed with DCM/MeOH (10/1, 100 mL×3) and the filtrate was concentrated in vacuo to give crude, which was purified with silica gel column chromatography eluting with a gradient of 0-5% MeOH in DCM to give (4-((1H-pyrazol-1-yl)methyl)phenyl)methanol (11.8 g, 80%) as a white solid. ESI-MS [M+H]⁺: 189.2.

Synthesis of 1-(4-(((6-chloropyridazin-4-yl)amino)methyl)benzyl)pyridin-2(1H)-one. To a solution of (4-((1H-pyrazol-1-yl)methyl)phenyl)methanol (11.8 g, 62.4 mmol) in THF (120 mL) was added NaH (2.7 g, 68.6 mmol, 60% dispersion in mineral oil) at 0° C. slowly. The reaction was stirred at 0° C. for 1 h, then a solution of 3,5-dichloropyridazine (9.3 g, 62.4 mmol) in THF (30 mL) was added thereto. The resulting mixture was stirred at room temperature for 1 h. The reaction was then quenched with NH₄Cl (sat. aq., 150 mL), extracted with EtOAc (150 mL×3). The combined organic layers were washed with brine (200 mL), dried over anhydrous Na₂SO₄, concentrated in vacuo to give the crude, which was purified with silica gel column chromatography eluting with a gradient of 0-50% EtOAc in PE to give 5-((4-((1H-pyrazol-1-yl)methyl)benzyl)oxy)-3-chloropyridazine (11.1 g, 59%) as a yellow solid. ESI-MS [M+H]⁺: 301.2.

Synthesis of N-(5-((4-((1H-pyrazol-1-yl)methyl)benzyl)oxy)pyridazin-3-yl)-1,1-diphenylmethanimine. A mixture of 5-((4-((1H-pyrazol-1-yl)methyl)benzyl)oxy)-3-chloropyridazine (5 g, 16.7 mmol), diphenylmethanimine (6 g, 33.4 mmol), Pd-PEPPSI-IPent-CI-o-picoline (706 mg, 0.84 mmol) and Cs₂CO₃ (16.3 mg, 50.1 mmol) in DMF (50 mL) was stirred at 110° C. for 16 h. The reaction was cooled to room temperature, diluted with H₂O (150 mL) and extracted with EtOAc (100 mL×3). The combined organic layers were washed with brine (80 mL), dried over anhydrous Na₂SO₄, then concentrated in vacuo to give crude, which was purified with silica gel column chromatography eluting with a gradient of 0-50% EtOAc in PE to give N-(5-((4-((1H-pyrazol-1-yl)methyl)benzyl)oxy)pyridazin-3-yl)-1,1-diphenylmethanimine (1.3 g, 18%) as a yellow solid. ESI-MS [M+H]⁺: 446.2.

Synthesis of 5-((4-((1H-pyrazol-1-yl)methyl)benzyl)oxy)pyridazin-3-amine. To a solution of N-(5-((4-((1H-pyrazol-1-yl)methyl)benzyl)oxy)pyridazin-3-yl)-1,1-diphenylmethanimine (1.3 g, 2.9 mmol) in MeOH (20 mL) was added HCl (4 M solution in MeOH, 5 mL). The resulting reaction was stirred at room temperature for 2 h. The reaction was concentrated in vacuo. The residue was neutralized with NH₃ (7 M solution in MeOH, 10 mL) and concentrated in vacuo to give the crude, which was purified with silica gel column chromatography eluting with a gradient of 0-10% MeOH (10% NH₃H₂O as additive) in DCM to 5-((4-((1H-pyrazol-1-yl)methyl)benzyl)oxy)pyridazin-3-amine (450 mg, 55%) as a yellow solid. ESI-MS [M+H]⁺: 282.2.

Synthesis of rac-(1S*,2S*)—N-(5-((4-((1H-pyrazol-1-yl)methyl)benzyl)oxy)pyridazin-3-yl)-2-(3-chlorophenyl)cyclopropane-1-carboxamide. A mixture of 5-((4-((1H-pyrazol-1-yl)methyl)benzyl)oxy)pyridazin-3-amine (56 mg, 0.2 mmol), rac-(1S*,2S*)-2-(3-chlorophenyl)cyclopropane-1-carboxylic acid (39 mg, 0.2 mmol), PyBOP (208 mg, 0.4 mmol) and DIPEA (129 mg, 1.0 mmol) in DMF (5 mL) was stirred at room temperature for 16 h. The reaction was quenched with water (50 mL) and extracted with EtOAc (20 mL×3). The combined organic layers were washed with brine (30 mL), dried over anhydrous Na₂SO₄, and concentrated in vacuo to give the crude product, which was purified by preparative HPLC to give rac-(1S*,2S*)—N-(5-((4-((1H-pyrazol-1-yl)methyl)benzyl)oxy)pyridazin-3-yl)-2-(3-chlorophenyl)cyclopropane-1-carboxamide (5 mg, 5%) as a white solid. ESI-MS [M+H]⁺: 460.2. ¹H NMR (400 MHz, DMSO) δ 11.35 (s, 1H), 8.76 (d, J=2.7 Hz, 1H), 8.02 (d, J=2.7 Hz, 1H), 7.83 (d, J=2.0 Hz, 1H), 7.50-7.40 (m, 3H), 7.33-7.16 (m, 6H), 6.27 (t, J=2.0 Hz, 1H), 5.35 (s, 2H), 5.22 (s, 2H), 2.47-2.41 (m, 2H), 1.53-1.45 (m, 2H).

### Example 19

Synthesis of rac-(1R^{•},2S*)-2-(3-chlorophenyl)-N-(5-((4-((2-oxopyridin-1 (2H)-yl)methyl)benzyl)amino)pyridazin-3-yl)cyclopropane-1-carboxamide (I-19)

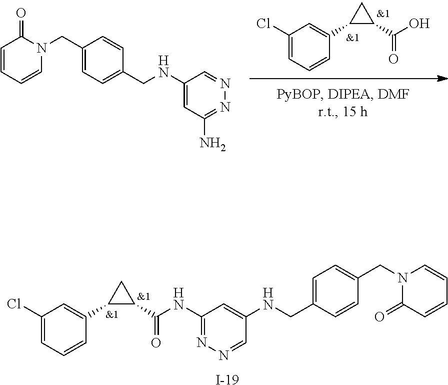

A solution of 1-(4-(((6-aminopyridazin-4-yl)amino)methyl)benzyl)pyridin-2 (1H)-one (50 mg, 0.16 mmol), rac-(1R*,2S*)-2-(3-chlorophenyl)cyclopropane-1-carboxylic acid (31 mg, 0.16 mmol), PyBop (125 mg, 0.24 mmol) and DIPEA (62 mg, 0.48 mmol) in DMF (2.0 mL) was stirred at room temperature overnight. The mixture was diluted with water (20 mL) and extracted with ethyl acetate (10 mL×3). The combined organic layers were washed with brine (20 mL) and concentrated in vacuo. The residue was purified by preparative HPLC to give rac-(1R*,2S*)-2-(3-chlorophenyl)-N-(5-((4-((2-oxopyridin-1 (2H)-yl)methyl)benzyl)amino)pyridazin-3-yl)cyclopropane-1-carboxamide (2.1 mg, 3%) as a white solid. ESI-MS (M+H)+: 486.2. ¹H NMR (400 MHz, MeOD) δ 8.33 (s, 1H), 8.11 (d. J=4 Hz, 1H), 7.57 (d, J=4 Hz, 1H), 7.45-7.40 (m, 1H), 7.17 (s, 5H), 7.07-7.04 (m, 3H), 6.99 (s, 1H), 5.08 (s, 2H), 4.18 (s, 2H), 2.57-2.51 (m, 1H), 2.29-2.24 (m, 1H), 1.65-1.60 (m, 1H), 1.32-1.23 (m, 1H).

### Example 20

### Synthesis of rac-(1S*,2S*)—N-(5-(1-(4-((5-chloro-2-oxopyridin-1 (2H)-yl)methyl) phenyl)cyclopropyl)pyridazin-3-yl)-2-(3-chlorophenyl)cyclopropane-1-carboxamide (I-20)

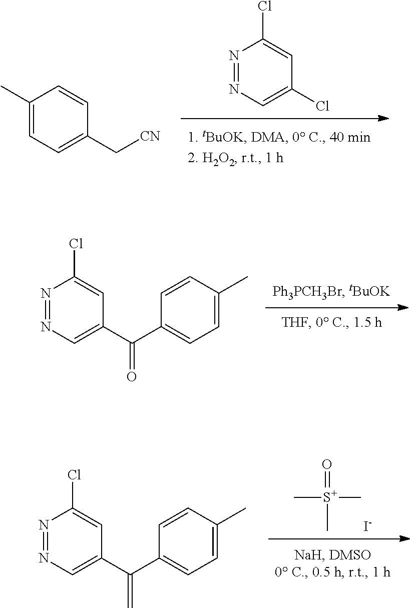

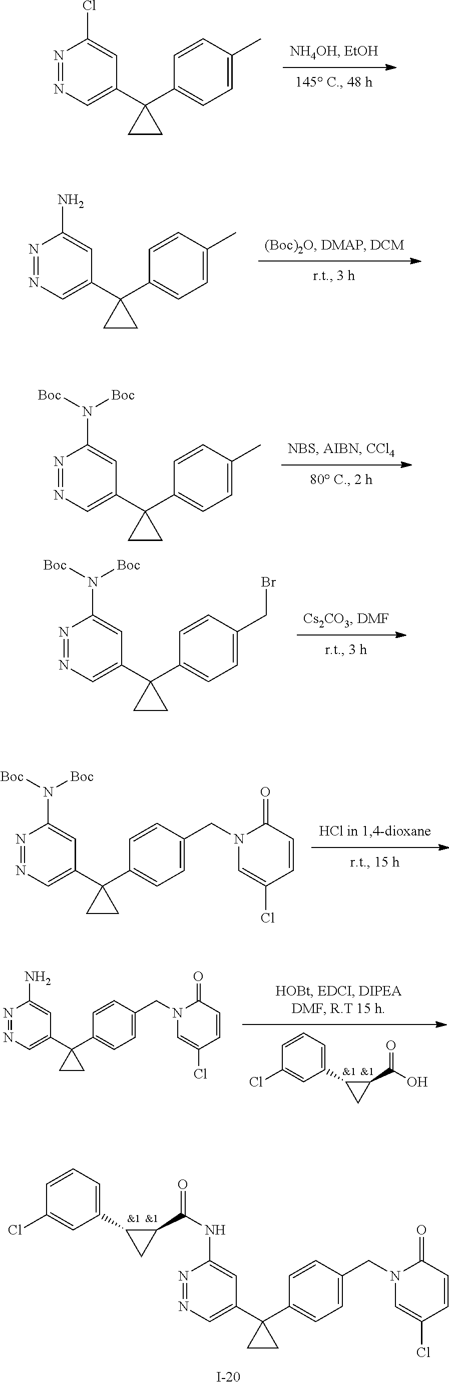

Synthesis of (6-chloropyridazin-4-yl)(p-tolyl)methanone. To a solution of 2-(p-tolyl)acetonitrile (5 g, 38 mmol) in DMA (20 mL) was added ^{t}BuOK (6.4 g, 57 mmol). After stirring at 0° C. for 10 min, to the mixture was added 3,5-dichloropyridazine (5.6 g, 38 mmol) and stirred for another 30 min at 0° C. The mixture was cooled to −78° C. and was added H₂O₂ (7.0 mL) dropwise. The resulting mixture was warmed to room temperature and stirred for another 1 h. The reaction mixture was then poured into water (150 mL) and extracted with EtOAc (30 mL×3). The combined organics were washed with brine (50 mL), dried over Na₂SO₄ and concentrated in vacuo. The residue was purified by column chromatography on silica gel, eluting with 10˜30% EtOAc in PE to give (6-chloropyridazin-4-yl)(p-tolyl)methanone (1.0 g, 11%) as a yellow oil. ESI-MS (M+H)+: 233.1.

Synthesis of 3-chloro-5-(1-(p-tolyl)vinyl)pyridazine. To a solution of methyltriphenylphosphonium bromide (1.5 g, 4.3 mmol) in dry THF (20 mL) was added tBuOK (729 mg, 6.5 mmol) at 0° C. After stirring at 0° C. for 30 min, to the resulting mixture was added (6-chloropyridazin-4-yl)(p-tolyl)methanone (1.0 g, 4.3 mmol) and resulting mixture was stirred for another 1 h. The mixture was poured into water (150 mL) and extracted with EtOAc (40 mL×3). The combined organics were washed with brine (50 mL), dried over Na₂SO₄ and concentrated in vacuo. The residue was purified by column chromatography on silica gel, eluting with 5˜20% EtOAc in PE to give 3-chloro-5-(1-(p-tolyl)vinyl)pyridazine (650 mg, 65%) as a white solid. ESI-MS (M+H)+: 231.1.

Synthesis of 3-chloro-5-(1-(p-tolyl)cyclopropyl)pyridazine. To a solution of trimethylsulfoxonium iodide (572 mg, 2.6 mmol) in DMSO (5.0 mL) was added NaH (156 mg, 60% in mineral oil, 3.9 mmol). After stirring at 0° C. for 30 min, the resulting mixture was added 3-chloro-5-(1-(p-tolyl)vinyl)pyridazine (600 mg, 2.6 mmol) and stirred at room temperature for another 3 h. The mixture was poured into water (100 mL) and extracted with EtOAc (20 mL×3). The combined organics were washed with brine (50 mL), dried over Na₂SO₄ and concentrated in vacuo. The residue was purified by column chromatography on silica gel, eluting with 5˜30% EtOAc in PE to give 3-chloro-5-(1-(p-tolyl)cyclopropyl)pyridazine (400 mg, 63%) as a yellow oil. ESI-MS (M+H)+: 245.2.

Synthesis of 5-(1-(p-tolyl)cyclopropyl)pyridazin-3-amine. A solution of 3-chloro-5-(1-(p-tolyl)cyclopropyl)pyridazine (400 mg, 1.63 mmol) and ammonium hydroxide (2.0 mL) in EtOH (2.0 mL) was stirred at 145° C. for 48 h. The mixture was cooled to room temperature and concentrated in vacuo. The residue was purified by column chromatography on silica gel, eluting with 0˜10% MeCN in H₂O to give 5-(1-(p-tolyl)cyclopropyl)pyridazin-3-amine (150 mg, 41%) as a yellow oil. ESI-MS (M+H)+: 226.0.

Synthesis of 5-(1-(p-tolyl)cyclopropyl)pyridazin-3-(N,N-di-tert-butyl carbonate)amine. A solution of 5-(1-(p-tolyl)cyclopropyl)pyridazin-3-amine (150 mg, 0.67 mmol), di-tert-butyl dicarbonate (439 mg, 2.01 mmol) and DMAP (8.2 mg, 0.067 mmol) in DCM (10 m) was stirred at room temperature for 3 h. The reaction was quenched with water (50 mL) and extracted with DCM (50 mL×3). The combined organic layers were washed with brine (50 mL), dried over Na₂SO₄ and concentrated in vacuo. The residue was purified by column chromatography on silica gel, eluting with 5˜30% EtOAc in PE to give 5-(1-(p-tolyl)cyclopropyl)pyridazin-3-(N,N-di-tert-butyl carbonate)amine (200 mg, 70%) as a yellow oil. ESI-MS (M+H)+: 426.2.

Synthesis of 5-(1-(4-(bromomethyl)phenyl)cyclopropyl)pyridazin-3-(N,N-ditert-butyl methyl)amine. A solution of 5-(1-(p-tolyl)cyclopropyl)pyridazin-3-(N,N-di-tert-butyl carbonate)amine (200 mg, 0.47 mmol), NBS (126 mg, 0.71 mmol) and AIBN (231 mg, 1.41 mmol) in CCl₄ (5.0 m-L) was stirred at 80° C. for 2 h. The mixture was cooled to room temperature and quenched with water (50 mL) and extracted with DCM (50 mL×3). The combined organic layers were washed with brine (50 mL), dried over Na₂SO₄ and concentrated in vacuo. The residue was purified by column chromatography on silica gel, eluting with 10˜40% EtOAc in PE to give 5-(1-(4-(bromomethyl)phenyl)cyclopropyl)pyridazin-3-(N,N-ditert-butyl methyl)amine (110 mg, 46%) as a yellow oil. ESI-MS (M+H)+: 504.1.

Synthesis of 1-(4-(1-(6-(N,N-ditert-butylmethyl)aminopyridazin-4-yl)cyclopropyl)benzyl) -5-chloropyridin-2 (1H)-one. A solution of 5-(1-(4-(bromomethyl)phenyl)cyclopropyl)pyridazin-3-(N,N-ditert-butyl methyl)amine (110 mg, 0.22 mmol), 5-chloropyridin-2 (1H)-one (43 mg, 0.33 mmol) and Cs₂CO₃ (215 mg, 0.66 mmol) in DMF (5 mL) was stirred at room temperature for 3 h. The mixture was quenched with water (30 mL) and extracted with EtOAc (30 mL×3). The combined organic layers were washed with brine (50 mL), dried over Na₂SO₄ and concentrated in vacuo. The residue was purified by column chromatography on silica gel, eluting with 5˜25% EtOAc in PE to give 1-(4-(1-(6-(N,N-ditert-butylmethyl)aminopyridazin-4-yl)cyclopropyl)benzyl)-5-chloropyridin-2 (1H)-one (80 mg, 66%) as a yellow oil. ESI-MS (M+H)+: 554.2.

Synthesis of 1-(4-(1-(6-aminopyridazin-4-yl)cyclopropyl)benzyl)-5-chloropyridin-2 (1H)-one. A solution of 1-(4-(1-(6-(N,N-ditert-butylmethyl)aminopyridazin-4-yl)cyclopropyl)benzyl)-5-chloropyridin-2 (1H)-one (80 mg, 0.15 mmol) and HCl (4 N in 1,4-dioxane, 2.0 mL) was stirred at room temperature overnight. The mixture was concentrated in vacuo to give 1-(4-(1-(6-aminopyridazin-4-yl)cyclopropyl)benzyl)-5-chloropyridin-2 (1H)-one (60 mg, crude) as a yellow oil. ESI-MS (M+H)+: 353.2.

Synthesis of rac-(1S*,2S*)—N-(5-(1-(4-((5-chloro-2-oxopyridin-1 (2H)-yl)methyl)phenyl)cyclopropyl)pyridazin-3-yl)-2-(3-chlorophenyl)cyclopropane-1-carboxamide. A mixture of 1-(4-(1-(6-aminopyridazin-4-yl)cyclopropyl)benzyl)-5-chloropyridin-2 (1H) -one (60 mg, crude), rac-(1S*,2S*)-2-(3-chlorophenyl)cyclopropane-1-carboxylic acid (22 mg, 0.11 mmol), HOBt (23 mg, 0.17 mmol), EDCI (63 mg, 0.33 mmol) and DIPEA (43 mg, 0.33 mmol) in DMF (2.0 mL) was stirred at room temperature overnight. Water (20 mL) was added and the mixture was extracted with EtOAc (20 mL×3). The combined organic layers were washed with brine (15 mL), dried over Na₂SO₄ and concentrated in vacuo. The residue was purified by column chromatography on silica gel, eluting with 20˜60% EtOAc in PE to give rac-(1S*,2S*)—N-(5-(1-(4-((5-chloro-2-oxopyridin-1 (2H)-yl)methyl)phenyl)cyclopropyl)pyridazin-3-yl)-2-(3-chlorophenyl)cyclopropane-1-carboxamide (3 mg, 5%) as a white solid. ESI-MS (M+H)+: 486.2. ¹H NMR (400 MHz, MeOD) δ 8.55 (s, 1H), 8.21 (s, 1H), 7.93 (dd, J=8 Hz, 10 Hz, 1H), 7.56-7.53 (m, 1H), 7.39 (s, 4H), 7.29 (t, J=8 Hz, 1H), 7.22 (d, J=8 Hz, 2H), 7.13 (d, J=8 Hz, 1H), 5.21 (s, 2H), 2.51 (s, 1H), 2.25-2.19 (m, 1H), 2.06 (s, 1H), 1.65-1.61 (m, 2H), 1.40-1.32 (m, 2H).

### Example 21

Synthesis of rac-(1S*,2S*)-2-(3-chlorophenyl)-N-(6-((3-cyclopropylquinolin-6-yl)methyl)pyrimidin-4-yl)cyclopropane-1-carboxamide (I-21)

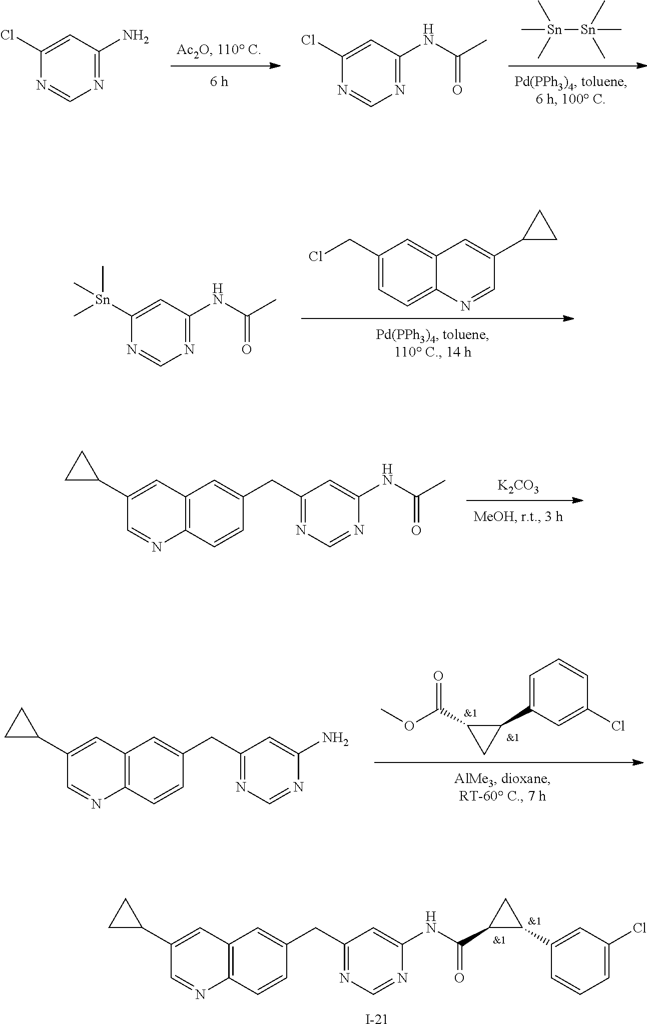

Synthesis of N-(6-chloropyrimidin-4-yl)acetamide. A mixture of 3-(chloromethyl)-6-cyclopropylquinoline (3.5 g, 27.1 mmol) in Ac₂O (30 mL) was stirred at 110° C. for 6 h. After cooling to room temperature, the reaction was quenched with NaHCO₃ (sat. aq., 200 mL) and extracted with DCM (100 mL×3). The combined organic layers were washed with brine (100 mL), dried over Na₂SO₄ and concentrated in vacuo to give N-(6-chloropyrimidin-4-yl)acetamide, which was purified by column chromatography (eluent: MeOH/DCM=0 to 5%) to give N-(6-chloropyrimidin-4-yl)acetamide (4.5 g, 97% yield) as a white solid. ESI-MS [M+H]⁺: 172.0.

Synthesis of N-(6-(trimethylstannyl)pyrimidin-4-yl)acetamide. A mixture of N-(6-chloropyrimidin-4-yl)acetamide (2.0 g, 11.7 mmol), Pd(PPh₃)₄ (1.4 g, 1.17 mmol) and 1,1,1,2,2,2-hexamethyldistannane (4.1 g, 12.3 mmol) in toluene (30 mL) was stirred at 100° C. for 6 h. After cooling to room temperature, the reaction mixture was filtered and the filtrate was concentrated in vacuo to give N-(6-(trimethylstannyl)pyrimidin-4-yl)acetamide (5.0 g, crude) a yellow oil which was used without further purification. ESI-MS [M+H]⁺: 302.0.

Synthesis of N-(6-((3-cyclopropylquinolin-6-yl)methyl)pyrimidin-4-yl)acetamide. A mixture of N-(6-(trimethylstannyl)pyrimidin-4-yl)acetamide (2.5 g, crude), 6-(chloromethyl)-3-cyclopropylquinoline (500 mg, 2.3 mmol) and Pd(PPh₃)₄ (269 mg, 0.23 mmol) in toluene (20 mL) was stirred at 110° C. for 14 h under N₂. After cooling to room temperature, the reaction was quenched with NaHCO₃ (sat. aq., 200 mL) and extracted with DCM (100 mL×3). The combined organic layers were washed with brine (100 mL), dried over Na₂SO₄ and concentrated in vacuo to give the crude, which was purified by column chromatography (eluent: MeOH/DCM=0 to 8%) to give N-(6-((3-cyclopropylquinolin-6-yl)methyl)pyrimidin-4-yl)acetamide (200 mg, 27%) as a brown solid. ESI-MS [M+H]⁺: 319.1.

Synthesis of 6-((3-cyclopropylquinolin-6-yl)methyl)pyrimidin-4-amine. A mixture of N-(6-((3-cyclopropylquinolin-6-yl)methyl)pyrimidin-4-yl)acetamide (200 mg, 0.63 mmol) and K₂CO₃ (174 mg, 1.26 mmol) in MeOH (10 mL) was stirred at room temperature for 3 h. The reaction was quenched with water (50 mL) and extracted with DCM (50 mL×3). The combined organic layers were washed with brine (50 mL), dried over Na₂SO₄ and concentrated in vacuo give the crude, which was purified by column chromatography (eluent: MeOH/DCM=0 to 8%) to give 6-((3-cyclopropylquinolin-6-yl)methyl)pyrimidin-4-amine (150 mg, 86% yield) as a yellow solid. ESI-MS [M+H]⁺: 277.1.

Synthesis of rac-(1S*,2S*)-2-(3-chlorophenyl)-N-(4-(((6-cyclopropylquinolin-3-yl)methyl)amino)pyridin-2-yl)cyclopropane-1-carboxamide. To a mixture of 6-((3-cyclopropylquinolin-6-yl)methyl)pyrimidin-4-amine (30 mg, 0.11 mmol) in 1,4-dioxane (3 mL ) was added AlMe₃ (0.21 mL, 0.33 mmol, 1.6 M in toluene) at room temperature and the mixture was stirred at room temperature for 0.5 h. rac-Methyl (1S*,2S*)-2-(3-chlorophenyl)cyclopropane-1-carboxylate (46 mg, 0.22 mmol) in 1,4-dioxane (1 mL ) was added and the mixture was stirred at 60° C. for 7 h under N₂. After cooling to room temperature, the reaction was quenched with NaHCO₃ (sat. aq., 30 mL) and extracted with EtOAc (50 mL×3). The combined organic layers were washed with brine (50 mL), dried over Na₂SO₄ and concentrated in vacuo to give the crude, which was purified by preparative TLC (eluent: MeOH/DCM=1/15) to give rac-(1S*,2S*)-2-(3-chlorophenyl)-N-(6-((3-cyclopropylquinolin-6-yl)methyl)pyrimidin-4-yl)cyclopropane-1-carboxamide (20 mg, 40% yield) as a white solid. ESI-MS [M+H]⁺: 455.2. ¹H NMR (400 MHz, DMSO) δ 11.16 (s, 1H), 8.79 (d, J=1.2 Hz, 1H), 8.73 (d, J=2.3 Hz, 1H), 7.99 (d, J=1.1 Hz, 1H), 7.93-7.88 (m, 2H), 7.73 (d, J=1.7 Hz, 1H), 7.59-7.54 (m, 1H), 7.35-7.22 (m, 3H), 7.17-7.10 (m, 1H), 4.23 (s, 2H), 2.44-2.32 (m, 2H), 2.18-2.07 (m, 1H), 1.51-1.42 (m, 2H), 1.09-1.04 (m, 2H), 0.91-0.85 (m, 2H).

### Example 22

### Synthesis of rac-(1S*,2S*)-2-(5-chloro-2-cyanophenyl)-N-(4-(((6-cyclopropylimidazo[1,2-a]pyrimidin-2-yl)methyl)amino)pyridin-2-yl)cyclopropane-1-carboxamide (1-22)

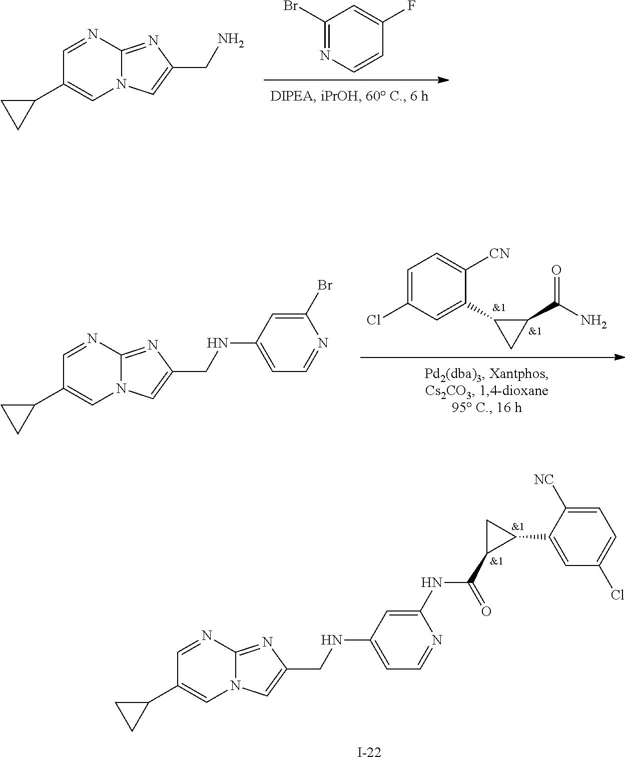

Synthesis of 2-bromo-N-((6-cyclopropylimidazo[1,2-a]pyrimidin-2-yl)methyl)pyridin-4-amine. To a solution of (6-cyclopropylimidazo[1,2-a]pyrimidin-2-yl)methanamine (77 mg, 0.41 mmol) and 2-bromo-4-fluoropyridine (105 mg, 0.60 mmol) in i-PrOH (10 mL) was added DIPEA (158.7 mg, 1.23 mmol). The reaction mixture was stirred at 60° C. for 6 under N₂. The reaction was concentrated in vacuo to give the crude, which was purified with preparative TLC (eluent: DCM/MeOH=10/1) to give 2-bromo-N-((6-cyclopropylimidazo[1,2-a]pyrimidin-2-yl)methyl)pyridin-4-amine (90 mg, 64%) as a yellow solid. ESI-MS [M+H]⁺: 344.1.

Synthesis of rac-(1S*,2S*)-2-(5-chloro-2-cyanophenyl)-N-(4-(((6-cyclopropylimidazo[1,2-a]pyrimidin-2-yl)methyl)amino)pyridin-2-yl)cyclopropane-1-carboxamide. To a solution of 2-bromo-N-((6-cyclopropylimidazo[1,2-a]pyrimidin-2-yl)methyl)pyridin-4-amine (90 mg, 0.26 mmol), rac-(1S*,2S*)-2-(5-chloro-2-cyanophenyl)cyclopropane-1-carboxamide (86 mg, 0.39 mmol), Pd₂(dba)₃ (24 mg, 0.026 mmol), Xantphos (15 mg, 0.026 mmol) and Cs₂CO₃ (254 mg, 0.78 mmol) in 1,4-dioxane (10 mL) was stirred at 95° C. for 16 h under nitrogen. The reaction was cooled to room temperature, diluted with H₂O (25 mL), and extracted with EtOAc (30 mL×3). The combined organic layers were washed with brine (30 mL), dried over anhydrous Na₂SO₄, and concentrated in vacuo to give the crude, which was purified by preparative TLC (eluent: DCM/MeOH=10/1) to give rac-(1S*,2S*)-2-(5-chloro-2-cyanophenyl)-N-(4-(((6-cyclopropylimidazo[1,2-a]pyrimidin-2-yl)methyl)amino)pyridin-2-yl)cyclopropane-1-carboxamide (10 mg, 8%) as a white solid. ESI-MS [M+H]⁺: 484.2. ¹H NMR (400 MHz, DMSO) δ 10.39 (s, 1H), 8.67 (d, J=2.2 Hz, 1H), 8.38 (d, J=2.4 Hz, 1H), 7.86 (d, J=8.3 Hz, 1H), 7.79-7.76 (m, 1H), 7.59 (s, 1H), 7.52-7.50 (m, 1H), 7.46 (s, 1H), 7.39 (d, J=1.7 Hz, 1H), 7.20 (t, J=5.7 Hz, 1H), 6.33 (d, J=3.9 Hz, 1H), 4.40 (d, J=5.8 Hz, 2H), 2.57-2.55 (m, 2H), 2.03-1.94 (m, 1H), 1.58-1.51 (m, 2H), 1.02-0.90 (m, 2H), 0.79-0.67 (m, 2H).

The compound in Table 7 was prepared in a similar manner to rac-(1S*,2S*)-2-(5-chloro-2-cyanophenyl)-N-(4-(((6-cyclopropylimidazo[1,2-a]pyrimidin-2-yl)methyl)amino)pyridin-2-yl)cyclopropane-1-carboxamide from 2-bromo-4-fluoropyridine and the indicated coupling partners.

TABLE 7

| | | Coupling | |
|---|---|---|---|
| EG | Structure | partners | Analytical data |
| 23 | 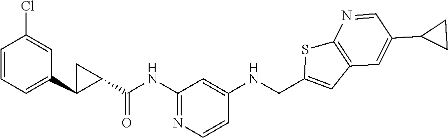 [SMILES: O=C(Nc1cc(NCc2cc3cc(C4CC4)cnc3s2)ccn1)[C@H]1C[C@@H]1c1cccc(Cl)c1] | 2-bromo-N-((5- cyclopropylthieno [2,3-b]pyridin- 2- yl)methyl)pyridin- 4-amine and (1S,2S)-2-(3- chlorophenyl)cy- clopropane-1- | ESI-MS (M + H)+: 475.3, Purity: 98.0 %, ¹H NMR (400 MHz, DMSO) δ 10.35 (s, 1H), 8.34 (d, J = 2.1 Hz, 1H), 8.14 (s, 0.6H), 7.78 (dd, J = 1.7, 3.9 Hz, 2H), 7.47 - 7.47 (m, 1H), 7.41 - 7.35 (m, 1H), 7.33 - 7.28 (m, 1H), 7.26 - 7.20 (m, 3H), 7.13 (ddd, J = 1.3, 1.3, 7.6 Hz, 1H), 6.34 (dd, J = 2.1, 5.8 Hz, |
| | (1S,2S)-2-(3-chlorophenyl)-N- | carboxamide | 1H), 4.59 (d, J = 5.7 Hz, 2H), 2.40 - |
| | (4-(((5-cyclopropylthieno[2,3- | | 2.32 (m, 2H), 2.09 - 2.01 (m, |
| | b]pyridin-2- | | 1H), 1.47 - 1.41 (m, 1H), 1.37 - |
| | yl)methyl)amino)pyridin-2- | | 1.31 (m, 1H), 1.04 - 0.98 (m, 2H), |
| | yl)cyclopropane-1- | | 0.79 - 0.74 (m, 2H). |
| | carboxamide, formic acid salt | | |
| | (I-23) | | |

### Example 24

### Synthesis of rac-(1S*,2S*)-2-(5-chloro-2-cyanophenyl)-N-(6-(((6-cyclopropyl-[1,2,4]triazolo[1,5-a]pyridin-2-yl)methyl)amino)pyrimidin-4-yl)cyclopropane-1-carboxamide (I-24)

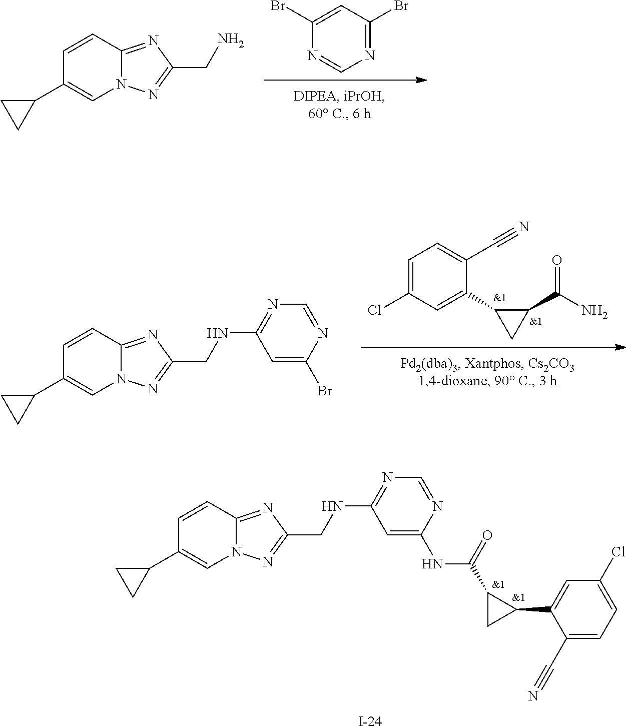

Synthesis of 6-bromo-N-((6-cyclopropyl-[1,2,4]triazolo[1,5-a]pyridin-2-yl)methyl)pyrimidin-4-amine. A mixture of (6-cyclopropyl-[1,2,4]triazolo[1,5-a]pyridin-2-yl)methanamine (140 mg, 0.74 mmol), 4,6-dibromopyrimidine (200 mg, 0.82 mmol) and DIPEA (477 mg, 3.7 mmol) in i-PrOH (6.0 mL) was stirred at 60° C. for 6 h. After cooling to room temperature, the reaction mixture was quenched with NaHCO₃ (sat. aq., 50 mL), extracted with DCM (30 ml×3). The combined organics were washed with brine (30 mL), dried over Na₂SO₄, and concentrated in vacuo to give the crude product, which was purified by column chromatography (eluent: EtOAc/PE=1/2) to give 6-bromo-N-((6-cyclopropyl-[1,2,4]triazolo[1,5-a]pyridin-2-yl)methyl)pyrimidin-4-amine (160 mg, 63%) as a white solid. ESI-MS [M+H]⁺: 345.0.

Synthesis of rac-(1S*,2S*)-2-(5-chloro-2-cyanophenyl)-N-(6-(((6-cyclopropyl-[1,2,4]triazolo[1,5-a]pyridin-2-yl)methyl)amino)pyrimidin-4-yl)cyclopropane-1-carboxamide. The mixture of 6-bromo-N-((6-cyclopropyl-[1,2,4]triazolo[1,5-a]pyridin-2-yl)methyl)pyrimidin-4-amine (75 mg, 0.22 mmol), rac-(1S*,2S*)-2-(5-chloro-2-cyanophenyl)cyclopropane-1-carboxamide (53 mg, 0.24 mmol), Pd₂(dba)₃ (20 mg, 0.022 mmol), Xantphos (25 mg, 0.044 mmol) and Cs₂CO₃ (215 mg, 0.66 mmol) in 1,4-dioxane (8.0 mL) was stirred at 90° C. for 3 h under nitrogen. After cooling to room temperature, the reaction mixture was filtered through Celite® and the filter cake was washed with DCM/MeOH (10/1, 50 mL). The filtrate was concentrated in vacuo to give the crude, which was purified by column chromatography (eluent: DCM/MeOH=10/1) and preparative HPLC to give rac-(1S*,2S*)-2-(5-chloro-2-cyanophenyl)-N-(6-(((6-cyclopropyl-[1,2,4]triazolo[1,5-a]pyridin-2-yl)methyl)amino)pyrimidin-4-yl)cyclopropane-1-carboxamide (40 mg, 38%) as a white solid. ESI-MS [M+H]⁺: 485.2. ¹H NMR (400 MHz, DMSO-d₆) δ 10.66 (s, 1H), 8.71 (s, 1H), 8.18 (d, J=0.7 Hz, 1H), 8.01 (s, 1H), 7.87 (d, J=8.3 Hz, 1H), 7.63 (d, J=9.3 Hz, 1H), 7.53 (dd, J=8.3, 2.0 Hz, 1H), 7.44-7.35 (m, 2H), 7.31 (s, 1H), 4.72 (s, 2H), 2.62-2.54 (m, 1H), 2.51-2.45 (m, 1H), 2.08-2.00 (m, 1H), 1.64-1.52 (m, 2H), 0.99-0.93 (m, 2H), 0.81-0.73 (m, 2H).

### Example 25

### Synthesis of (1S,2S)-2-(3-chlorophenyl)-N-(4-(((6-cyclopropyl-[1,2,4]triazolo[1,5-a]pyridin-2-yl)methyl)amino)pyridin-2-yl)cyclopropane-1-carboxamide (1-25)

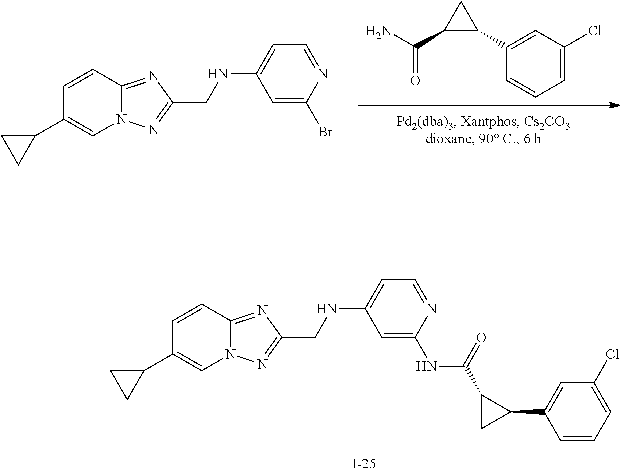

A mixture of 2-bromo-N-((6-cyclopropyl-[1,2,4]triazolo[1,5-a]pyridin-2-yl)methyl)pyridin-4-amine (53 mg, 0.15 mmol, prepared in a similar manner to 2-bromo-N-((6-cyclopropylimidazo[1,2-a]pyrimidin-2-yl)methyl)pyridin-4-amine from 2-bromo-4-fluoropyridine and (6-cyclopropyl-[1,2,4]triazolo[1,5-a]pyridin-2-yl)methanamine), (1S,2S)-2-(3-chlorophenyl)cyclopropane-1-carboxamide (29 mg, 0.15 mmol), Pd₂(dba)₃ (14 mg, 0.015 mmol), Xantphos (17 mg, 0.030 mmol) and Cs₂CO₃ (147 mg, 0.45 mmol) in 1,4-dioxane (5.0 mL) was stirred at 90° C. for 3 h under nitrogen. After cooling to room temperature, the reaction mixture was filtered through Celite® and the filter cake was washed with DCM/MeOH (10/1, 50 mL). The filtrate was concentrated in vacuo to give the crude, which was purified by (eluent: DCM/MeOH=10/1) and preparative TLC (eluent: DCM/MeOH=10/1) to give (1S,2S)-2-(3-chlorophenyl)-N-(4-(((6-cyclopropyl-[1,2,4]triazolo[1,5-a]pyridin-2-yl)methyl)amino)pyridin-2-yl)cyclopropane-1-carboxamide (30 mg, 43%) as a white solid. ESI-MS [M+H]⁺: 459.2. ¹H NMR (400 MHz, DMSO-d₆) δ 10.31 (s, 1H), 8.72 (s, 1H), 7.76 (d, J=5.8 Hz, 1H), 7.65 (d, J=9.2 Hz, 1H), 7.43-7.37 (m, 2H), 7.33-7.28 (m, 1H), 7.27-7.20 (m, 3H), 7.16-7.11 (m, 1H), 6.35 (dd, J=5.8, 2.1 Hz, 1H), 4.48 (d, J=6.1 Hz, 2H), 2.39-2.31 (m, 2H), 2.06-2.00 (m, 1H), 1.47-1.41 (m, 1H), 1.37-1.32 (m, 1H), 1.01-0.94 (m, 2H), 0.81-0.74 (m, 2H).

### Example 26

### Synthesis of (1S,2S)-2-(3-chlorophenyl)-N-(6-(((6-cyclopropyl-[1,2,4]triazolo[1,5-a]pyridin-2-yl)methyl)amino)pyrimidin-4-yl)cyclopropane-1-carboxamide (I-26)

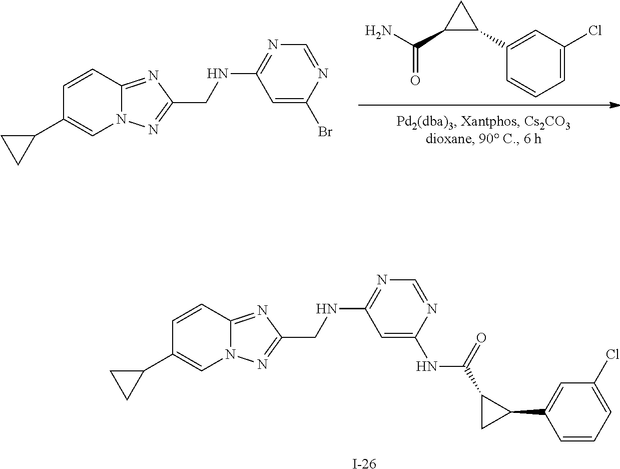

A mixture of 6-bromo-N-((6-cyclopropyl-[1,2,4]triazolo[1,5-a]pyridin-2-yl)methyl)pyrimidin-4-amine (53 mg, 0.15 mmol), (1S,2S)-2-(3-chlorophenyl)cyclopropane-1-carboxamide (29 mg, 0.15 mmol), Pd₂(dba)₃ (14 mg, 0.015 mmol), Xantphos (17 mg, 0.030 mmol) and Cs₂CO₃ (147 mg, 0.45 mmol) in 1,4-dioxane (5.0 mL) was stirred at 90° C. for 3 h under nitrogen. After cooling to room temperature, the reaction mixture was filtered through Celite® and the filter cake was washed with DCM/MeOH (10/1, 50 mL). The filtrate was concentrated in vacuo to give the crude, which was purified by column chromatography (eluent: DCM/MeOH=10/1) and preparative TLC (eluent: DCM/MeOH=10/1) to give the desired product (45 mg, 65%) as a white solid. ESI-MS [M+H]⁺: 460.2. ¹H NMR (400 MHz, DMSO-d₆) δ 10.60 (s, 1H), 8.70 (s, 1H), 8.17 (s, 1H), 7.98 (s, 1H), 7.63 (d, J=9.2 Hz, 1H), 7.38 (dd, J=9.2, 1.7 Hz, 1H), 7.35-7.23 (m, 4H), 7.15 (d, J=7.6 Hz, 1H), 4.71 (s, 2H), 2.44-2.35 (m, 2H), 2.06-2.00 (m, 1H), 1.51-1.44 (m, 1H), 1.43-1.38 (m, 1H), 1.00-0.94 (m, 2H), 0.80-0.75 (m, 2H).

### Example 27

### Synthesis of rac-(1S*,2S*)-2-(5-chloro-2-cyanophenyl)-N-(6-(((6-cyclopropyl-[1,2,4]triazolo[1,5-b]pyridazin-2-yl)methyl)amino)pyrimidin-4-yl)cyclopropane-1-carboxamide (I-27)

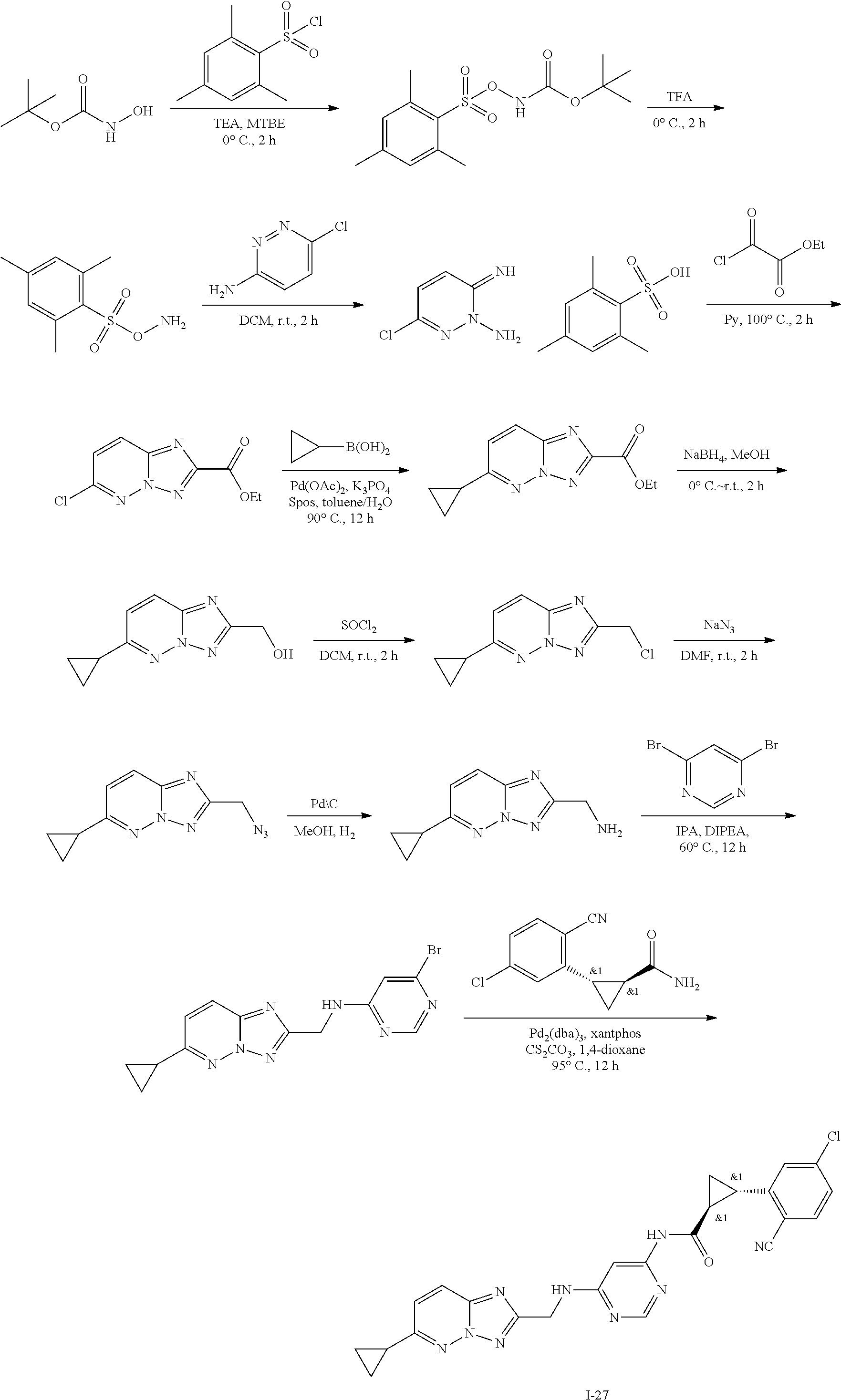

Synthesis of tert-butyl ((mesitylsulfonyl)oxy)carbamate. To a solution of tert-butyl hydroxycarbamate (12.0 g, 90 mmol) in MTBE (400 mL) was added 2,4,6-trimethylbenzenesulfonyl chloride (19.7 g, 90 mmol) and TEA (10.0 g, 99 mmol). The mixture was stirred at 0° C. for 2 h. The reaction mixture was filtered and the filtrate was concentrated in vacuo to give tert-butyl ((mesitylsulfonyl)oxy)carbamate (28 g, quant) as a yellow solid, which was used in the next step without further purification. ESI-MS [M+H]⁺: 316.1.

Synthesis of O-(mesitylsulfonyl)hydroxylamine. Tert-butyl ((mesitylsulfonyl)oxy)carbamate (28 g, 89 mmol) was added in TFA (150 ml) slowly at 0° C. and the mixture was stirred at 0° C. for 2 h. The mixture was poured into ice water (200 mL) and stirred for 10 min. The precipitate was filtered and dried in vacuo to give O-(mesitylsulfonyl)hydroxylamine (6.8 g, 36%) as a white solid. ESI-MS [M+H]⁺: 216.1.

Synthesis of 3-chloro-6-iminopyridazin-1 (6H)-amine 2,4,6-trimethylbenzenesulfonate. To a solution of 6-chloropyridazin-3-amine (450 mg, 3.47 mmol) in DCM (4 mL) was added 0-(mesitylsulfonyl)hydroxylamine (746 mg, 3.47 mmol) and the mixture was stirred at room temperature for 2 h. The reaction mixture was filtered and the filter cake was dried to give 3-chloro-6-iminopyridazin-1 (6H)-amine 2,4,6-trimethylbenzenesulfonate (1.2 g, quant) as a white solid, which was used in the next step without further purification. ESI-MS [M+H]⁺: 145.1.

Synthesis of ethyl 6-chloro-[1,2,4]triazolo[1,5-b]pyridazine-2-carboxylate. To a solution of 3-chloro-6-iminopyridazin-1 (6H)-amine 2,4,6-trimethylbenzenesulfonate (100 mg, 0.29 mmol) in pyridine (2 mL) was added ethyl 2-chloro-2-oxoacetate (79 mg, 0.58 mmol) and the mixture was stirred at 100° C. for 2 h. After cooling to room temperature, the reaction was quenched with NaHCO₃ (sat. aq., 30 mL) and extracted with DCM (30 mL×3). The combined organic layers were washed with brine (30 mL), dried over Na₂SO₄ and concentrated in vacuo to give the crude, which was purified by column chromatography (eluent: DCM/MeOH=1/50) to give ethyl 6-chloro-[1,2,4]triazolo[1,5-b]pyridazine-2-carboxylate (40 mg, 61%) as a white solid. ESI-MS [M+H]⁺: 227.1.

Synthesis of ethyl 6-cyclopropyl-[1,2,4]triazolo[1,5-b]pyridazine-2-carboxylate. To a mixture of ethyl 6-chloro-[1,2,4]triazolo[1,5-b]pyridazine-2-carboxylate (522 mg, 2.3 mmol), cyclopropylboronic acid (593 mg, 6.9 mmol) and K₃PO₄ (1.46 g, 6.9 mmol) in H₂O (1.2 ml) and toluene (12 mL) was added Pd(OAc)₂ (52 mg, 0.23 mmol) and S-Phos (94 mg, 0.23 mmol). The mixture was stirred at 95° C. for 12 h under N₂. After cooling to room temperature, the reaction was quenched with water (30 mL) and extracted with EtOAc (20 mL×3). The combined organic layers were washed with brine (30 mL), dried over Na₂SO₄, concentrated to give the crude, which was purified by column chromatography (eluent: DCM/MeOH=30/1) to give ethyl 6-cyclopropyl-[1,2,4]triazolo[1,5-b]pyridazine-2-carboxylate (300 mg, 56%) as a gray solid. ESI-MS [M+H]⁺: 233.1.

Synthesis of ethyl 6-cyclopropyl-[1,2,4]triazolo[1,5-b]pyridazine-2-carboxylate. To a solution of ethyl 6-cyclopropyl-[1,2,4]triazolo[1,5-b]pyridazine-2-carboxylate (280 mg, 1.2 mmol) in MeOH (10 mL) was added NaBH₄ (137 mg, 3.6 mmol) portionwise and the mixture was stirred at room temperature for 2 h. The reaction mixture was quenched with NH₄Cl (sat. aq., 50 mL), extracted with DCM (30 ml×3). The combined organics were washed with brine (30 mL), dried over Na₂SO₄ and concentrated in vacuo to give the crude product, which was purified by column chromatography (eluent: DCM/MeOH=20/1) to give (6-cyclopropyl-[1,2,4]triazolo[1,5-b]pyridazin-2-yl)methanol (120 mg, 53%) as a gray solid. ESI-MS [M+H]⁺: 191.1.

Synthesis of (6-cyclopropyl-[1,2,4]triazolo[1,5-b]pyridazin-2-yl)methanol. To a solution of (6-cyclopropyl-[1,2,4]triazolo[1,5-b]pyridazin-2-yl)methanol (200 mg, 1.05 mmol) in DCM (8 mL) was added SOCl₂ (375 mg, 3.15 mmol) slowly at 0° C. The mixture was stirred at room temperature for 2 h, then concentrated in vacuo to give 2-(chloromethyl)-6-cyclopropyl-[1,2,4]triazolo[1,5-b]pyridazine (220 mg, quant) as a yellow solid which was used in the next step without further purification. ESI-MS [M+H]⁺: 209.1.

Synthesis of 2-(azidomethyl)-6-cyclopropyl-[1,2,4]triazolo[1,5-b]pyridazine. To a solution of 2-(chloromethyl)-6-cyclopropyl-[1,2,4]triazolo[1,5-b]pyridazine (220 mg, 1.05 mmol) in DMF (4 mL) was added NaN₃ (103 mg, 1.58 mmol). The mixture was stirred at room temperature for 2 h. The reaction was quenched with water (50 mL) and extracted with EtOAc (30 mL×3). The combined organic layers were washed with brine (30 mL), dried over Na₂SO₄, and concentrated in vacuo to give the crude, which was purified by column chromatography (eluent: DCM/MeOH=20/1) to give 2-(azidomethyl)-6-cyclopropyl-[1,2,4]triazolo[1,5-b]pyridazine (182 mg, 81%) as a gray solid. ESI-MS [M+H]⁺: 216.1.

Synthesis of 2-(azidomethyl)-6-cyclopropyl-[1,2,4]triazolo[1,5-b]pyridazine. A mixture of 2-(azidomethyl)-6-cyclopropyl-[1,2,4]triazolo[1,5-b]pyridazine (180 mg, 0.84 mmol) and Pd\C (40 mg) in MeOH (5 mL) was stirred at 60° C. for 30 min. After cooling to room temperature, the reaction mixture was filtered and the filtrate was concentrated in vacuo to give (6-cyclopropyl-[1,2,4]triazolo[1,5-b]pyridazin-2-yl)methanamine (160 mg, quant) as a gray solid. which was used in the next step without further purification. ESI-MS [M+H]⁺: 190.2.

Synthesis of 6-bromo-N-((6-cyclopropyl-[1,2,4]triazolo[1,5-b]pyridazin-2-yl)methyl)pyrimidin-4-amine. A mixture of (6-cyclopropyl-[1,2,4]triazolo[1,5-b]pyridazin-2-yl)methanamine (60 mg, 0.32 mmol), 4,6-dibromopyrimidine (76 mg, 0.32 mmol) and DIPEA (124 mg, 0.96 mmol) in IPA (4 ml) was stirred at 60° C. for 4 h. After cooling to room temperature, the reaction mixture was poured into water (20 mL) and extracted with EtOAc (20 ml×3). The combined organics were washed with brine (20 mL), dried over Na₂SO₄ and concentrated in vacuo to give the crude product, which was purified by Pre-TLC (eluent: DCM/MeOH=15/1) to give the (66 mg, 60%) as a yellow solid. ESI-MS [M+H]⁺: 346.2.

Synthesis of rac-(1S*,2S*)-2-(5-chloro-2-cyanophenyl)-N-(6-(((6-cyclopropyl-[1,2,4]triazolo[1,5-b]pyridazin-2-yl)methyl)amino)pyrimidin-4-yl)cyclopropane-1-carboxamide. To a mixture of 6-bromo-N-((6-cyclopropyl-[1,2,4]triazolo[1,5-b]pyridazin-2-yl)methyl)pyrimidin-4-amine (40 mg, 0.12 mmol), rac-(1S*,2S*)-2-(5-chloro-2-cyanophenyl)cyclopropane-1-carboxamide (26 mg, 0.12 mmol) and Cs₂CO₃ (117 mg, 0.36 mmol) in 1,4-dioxane (5 mL) was added Pd₂(dba)₃ (16 mg, 0.018 mmol), XantPhos (14 mg, 0.024 mmol) and the mixture was stirred at 95° C. for 12 h under N₂. After cooling to room temperature, the reaction mixture was filtered through Celite® and the filter cake was washed with DCM/MeOH (10/1, 50 mL). The filtrate was concentrated in vacuo to give the crude product, which was purified by preparative TLC (eluent: DCM/MeOH=20/1) to give rac-(1S*,2S*)-2-(5-chloro-2-cyanophenyl)-N-(6-(((6-cyclopropyl-[1,2,4]triazolo[1,5-b]pyridazin-2-yl)methyl)amino)pyrimidin-4-yl)cyclopropane-1-carboxamide (20 mg, 34%) as a white solid. ESI-MS [M+H]⁺: 486.2., ¹H NMR (400 MHz, DMSO) δ 10.67 (s, 1H), 8.25-8.15 (m, 2H), 8.05 (s, 1H), 7.86 (d, J=8.3 Hz, 1H), 7.55-7.52 (m, 2H), 7.41 (d, J=1.8 Hz, 1H), 7.32 (s, 1H), 4.75 (s, 2H), 2.57-2.62 (m, 2H), 2.24-2.31 (m, 1H), 1.57-1.59 (m, 2H), 1.19-1.07 (m, 2H), 1.09-0.99 (m, 2H).

### Example 28

### Synthesis of (1S,2S)-2-(5-chloro-2-cyanophenyl)-N-(6-(((6-cyclopropyl-[1,2,4]triazolo[1,5-b]pyridazin-2-yl)methyl)amino)pyrimidin-4-yl)cyclopropane-1-carboxamide (I-28)

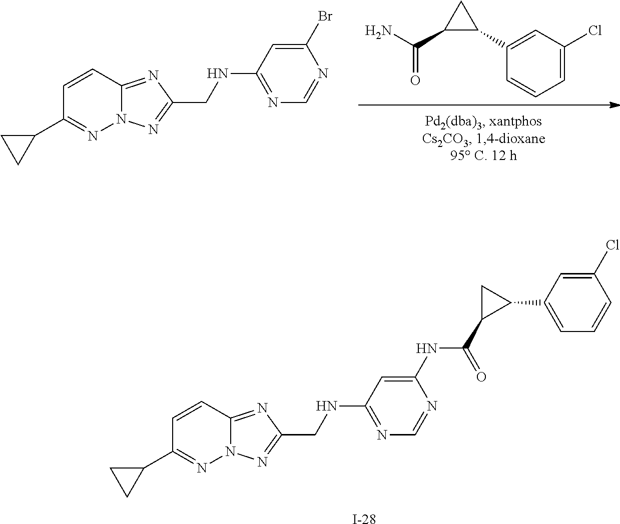

A mixture of 6-bromo-N-((6-cyclopropyl-[1,2,4]triazolo[1,5-b]pyridazin-2-yl)methyl)pyrimidin-4-amine (40 mg, 0.12 mmol), (1S,2S)-2-(3-chlorophenyl)cyclopropane-1-carboxamide (23 mg, 0.12 mmol), Pd₂(dba)₃ (16 mg, 0.018 mmol), xantphos (14 mg, 0.024 mmol), Cs₂CO₃ (117 mg, 0.36 mmol) in 1,4-dioxane (5 mL) was stirred at 95° C. for 12 h. After cooling to room temperature, the reaction mixture was filtered through Celite® and the filter cake was washed with DCM/MeOH (10/1, 100 mL). The filtrate was concentrated in vacuo to give the crude, which was purified by column chromatography (eluent: DCM/MeOH=50/1˜ 30/1) to give (1S,2S)-2-(3-chlorophenyl)-N-(6-(((6-cyclopropyl-[1,2,4]triazolo[1,5-b]pyridazin-2-yl)methyl)amino)pyrimidin-4-yl)cyclopropane-1-carboxamide (24 mg, 44%) as a white solid. ESI-MS [M+H]⁺: 461.2. ¹H NMR (400 MHz, DMSO) δ 10.62 (s, 1H), 8.20 (d, J=9.3 Hz, 1H), 8.17 (s, 1H), 8.02 (s, 1H), 7.53 (d, J=9.3 Hz, 1H), 7.30-7.34 (m, 2H), 7.24-7.28 (m, 2H), 7.15 (d, J=7.6 Hz, 1H), 4.74 (s, 2H), 2.40-2.51 (m, 2H), 2.28-2.36 (m, 1H), 1.51-1.36 (m, 2H), 1.18-1.08 (m, 2H), 1.06-0.99 (m, 2H).

### Example 29

### Synthesis of (1S,2S)-2-(3-chlorophenyl)-N-(4-(((6-cyclopropyl-[1,2,4]triazolo[1,5-b]pyridazin-2-yl)methyl)amino)pyridin-2-yl)cyclopropane-1-carboxamide (I-29)

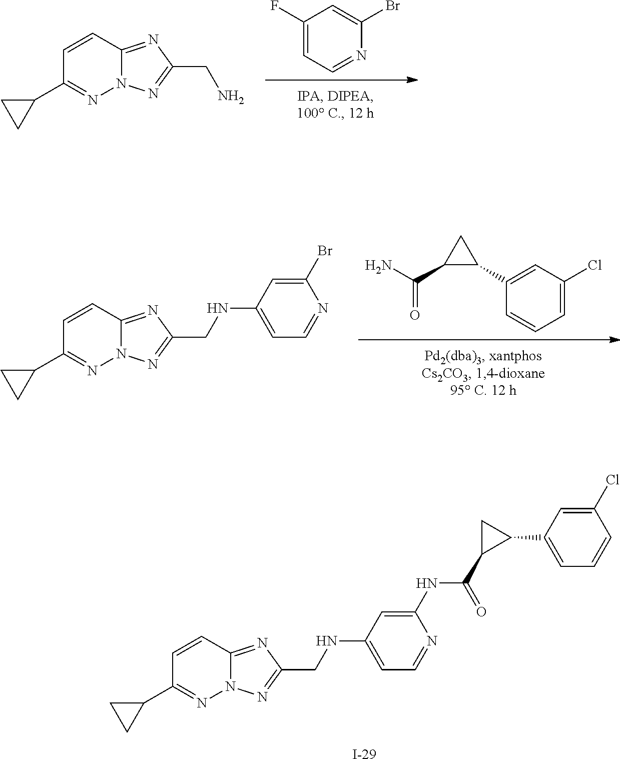

Synthesis of 2-bromo-N-((6-cyclopropyl-[1,2,4]triazolo[1,5-b]pyridazin-2-yl)methyl)pyridin-4-amine. A mixture of (6-cyclopropyl-[1,2,4]triazolo[1,5-b]pyridazin-2-yl)methanamine (60 mg, 0.32 mmol), 2-bromo-4-fluoropyridine (67 mg, 0.38 mmol) and DIPEA (123.8 mg, 0.96 mmol) in i-PrOH (4 ml) was stirred at 100° C. for 12 h. After cooling to room temperature, the reaction was quenched with water (30 mL) and extracted with DCM (30 mL×3). The combined organic layers were washed with brine (30 mL), dried over Na₂SO₄ then concentrated in vacuo to give the crude, which was purified by preparative TLC (eluent: MeOH/DCM=1/20) to give 2-bromo-N-((6-cyclopropyl-[1,2,4]triazolo[1,5-b]pyridazin-2-yl)methyl)pyridin-4-amine (50 mg, 45%) as a yellow solid. ESI-MS [M+H]⁺: 345.2.

Synthesis of (1S,2S)-2-(3-chlorophenyl)-N-(4-(((6-cyclopropyl-[1,2,4]triazolo[1,5-b]pyridazin-2-yl)methyl)amino)pyridin-2-yl)cyclopropane-1-carboxamide. To a mixture of 2-bromo-N-((6-cyclopropyl-[1,2,4]triazolo[1,5-b]pyridazin-2-yl)methyl)pyridin-4-amine (50 mg, 0.15 mmol), (1S,2S)-2-(3-chlorophenyl)cyclopropane-1-carboxamide (29 mg, 0.15 mmol) and Cs₂CO₃ (147 mg, 0.45 mmol) in 1,4-dioxane (5 mL) was added Pd₂(dba)₃ (21 mg, 0.023 mmol), xantPhos (17 mg, 0.030 mmol) and the mixture was stirred at 95° C. for 12 h under N₂. After cooling to room temperature, the reaction mixture was filtered through Celite® and the filter cake was washed with DCM/MeOH (10/1, 50 mL). The filtrate was concentrated to give the crude, which was purified by preparative TLC (eluent: DCM/MeOH=30/1) to give (1S,2S)-2-(3-chlorophenyl)-N-(4-(((6-cyclopropyl-[1,2,4]triazolo[1,5-b]pyridazin-2-yl)methyl)amino)pyridin-2-yl)cyclopropane-1-carboxamide (13 mg, 19%) as a white solid. ESI-MS [M+H]⁺: 460.2, ¹H NMR (400 MHz, DMSO) δ 10.32 (s, 1H), 8.22 (d, J=9.3 Hz, 1H), 7.77 (d, J=5.8 Hz, 1H), 7.52 (d, J=20.1, 1H), 7.43 (s, 1H), 7.33-7.19 (m, 4H), 7.21-7.08 (m, 1H), 6.38-6.34 (m, 1H), 4.52 (d, J=6.1 Hz, 2H), 2.39-2.33 (m, 2H), 2.31-2.23 (m, 1H), 1.48-1.41 (m, 1H), 1.38-1.31 (m, 1H), 1.17-1.10 (m, 2H), 1.07-1.01 (m, 2H).

### Example 30

### Synthesis of rac-(1S*,2S*)-2-(5-chloro-2-cyanophenyl)-N-(4-(((6-cyclopropyl-[1,2,4]triazolo[1,5-a]pyridin-2-yl)methyl)amino)pyridin-2-yl)cyclopropane-1-carboxamide (I-30)

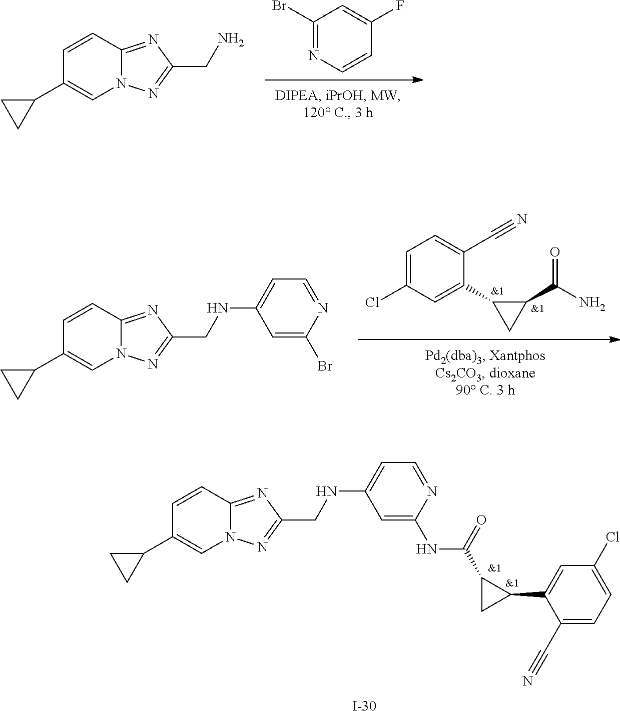

Synthesis of 2-bromo-N-((6-cyclopropyl-[1,2,4]triazolo[1,5-a]pyridin-2-yl)methyl)pyridin-4-amine. A mixture of (6-cyclopropyl-[1,2,4]triazolo[1,5-a]pyridin-2-yl)methanamine (280 mg, 1.5 mmol), 2-bromo-4-fluoropyridine (282 mg, 1.6 mmol) and DIPEA (968 mg, 7.5 mmol) in i-PrOH (10 mL) was stirred in a sealed tube. After degassing with N₂ for 1 min, the reaction was irradiated in a microwave reactor at 120° C. for 3 h. After cooling to room temperature, the reaction mixture was concentrated in vacuo. The residue was diluted with EtOAc (60 mL), washed with water (50 mL) and brine (50 mL), dried over Na₂SO₄ then concentrated in vacuo to give the crude product, which was purified by column chromatography (eluent: EtOAc/PE=1/1) to give 2-bromo-N-((6-cyclopropyl-[1,2,4]triazolo[1,5-a]pyridin-2-yl)methyl)pyridin-4-amine (190 mg, 37%) as a white solid. ESI-MS [M+H]⁺: 344.0.

Synthesis of rac-(1S*,2S*)-2-(5-chloro-2-cyanophenyl)-N-(4-(((6-cyclopropyl-[1,2,4]triazolo[1,5-a]pyridin-2-yl)methyl)amino)pyridin-2-yl)cyclopropane-1-carboxamide. A mixture of 2-bromo-N-((6-cyclopropyl-[1,2,4]triazolo[1,5-a]pyridin-2-yl)methyl)pyridin-4-amine (70 mg, 0.20 mmol), rac-(1S*,2S*)-2-(5-chloro-2-cyanophenyl)cyclopropane-1-carboxamide (44 mg, 0.20 mmol), Pd₂(dba)₃ (21 mg, 0.02 mmol), Xantphos (23 mg, 0.04 mmol) and Cs₂CO₃ (196 mg, 0.60 mmol) in 1,4-dioxane (8.0 mL) was stirred at 90° C. for 3 h under nitrogen. After cooling to room temperature, the reaction mixture was filtered through Celite® and the filter cake was washed with DCM/MeOH (10/1, 100 mL). The filtrate was concentrated in vacuo to give the crude, which was purified by preparative HPLC to give rac-(1S*,2S*)-2-(5-chloro-2-cyanophenyl)-N-(4-(((6-cyclopropyl-[1,2,4]triazolo[1,5-a]pyridin-2-yl)methyl)amino)pyridin-2-yl)cyclopropane-1-carboxamide (35 mg, 36%) as a white solid. ESI-MS [M+H]⁺: 484.2. ¹H NMR (400 MHz, DMSO-d₆) δ 10.36 (s, 1H), 8.72 (s, 1H), 7.85 (d, J=8.3 Hz, 1H), 7.77 (d, J=5.8 Hz, 1H), 7.65 (d, J=9.2 Hz, 1H), 7.51 (dd, J=8.3, 2.0 Hz, 1H), 7.44 (s, 1H), 7.41-7.36 (m, 2H), 7.25 (t, J=6.0 Hz, 1H), 6.36 (dd, J=5.8, 2.2 Hz, 1H), 4.49 (d, J=6.1 Hz, 2H), 2.58-2.53 (m, 1H), 2.48-2.44 (m, 1H), 2.07-1.99 (m, 1H), 1.58-1.49 (m, 2H), 1.00-0.94 (m, 2H), 0.81-0.75 (m, 2H).

### Example 31

### Synthesis of (1S,2S)-2-(3-chlorophenyl)-N-(4-(((6-cyclopropyl-[1,2,4]triazolo[1,5-a]pyrimidin-2-yl)methyl amino)pyridin-2-yl)cyclopropane-1-carboxamide 1-31)

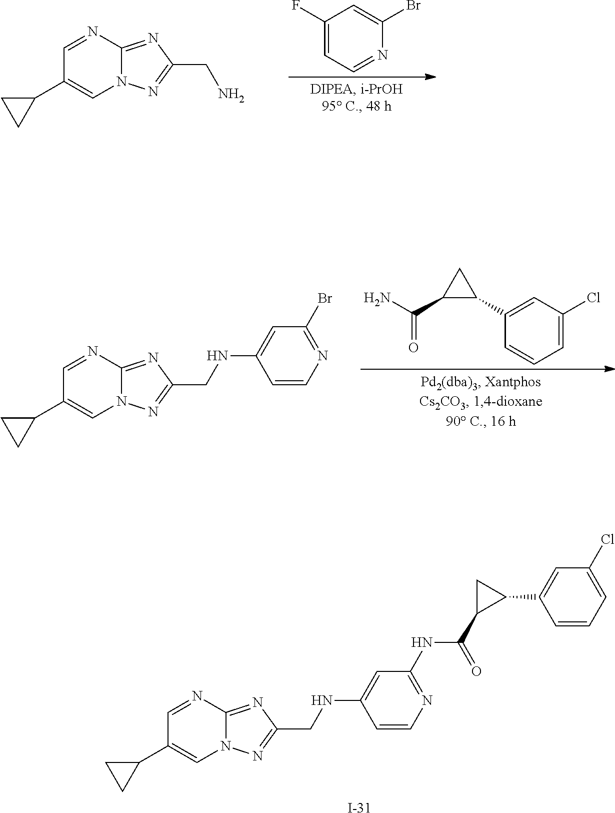 [SMILES: NCc1nc2ncc(C3CC3)cn2n1 ; Fc1ccnc(Br)c1 ; Brc1cc(NCc2nc3ncc(C4CC4)cn3n2)ccn1 ; NC(=O)[C@H]1C[C@@H]1c1cccc(Cl)c1 ; O=C(Nc1cc(NCc2nc3ncc(C4CC4)cn3n2)ccn1)[C@H]1C[C@@H]1c1cccc(Cl)c1 ; "I-31"]

Synthesis of 2-bromo-N-((6-cyclopropyl-[1,2,4]triazolo[1,5-a]pyrimidin-2-yl)methyl)pyridin-4-amine. A mixture of (6-cyclopropyl-[1,2,4]triazolo[1,5-a]pyrimidin-2-yl)methanamine (60 mg, 0.32 mmol), 2-bromo-4-fluoropyridine (56 mg, 0.32 mmol) and DIPEA (206 mg, 1.6 mmol) in i-PrOH (5 mL) was stirred at 95° C. for 48 h. The mixture was concentrated in vacuo to give the crude product, which was purified by preparative TLC (eluent: DCM/MeOH=10/1) to give 2-bromo-N-((6-cyclopropyl-[1,2,4]triazolo[1,5-a]pyrimidin-2-yl)methyl)pyridin-4-amine (30 mg, 27%) as a yellow solid. ESI-MS [M+H]⁺: 345.2.

Synthesis of (1S,2S)-2-(3-chlorophenyl)-N-(4-(((6-cyclopropyl-[1,2,4]triazolo[1,5-a]pyrimidin-2-yl)methyl)amino)pyridin-2-yl)cyclopropane-1-carboxamide. To a mixture of 2-bromo-N-((6-cyclopropyl-[1,2,4]triazolo[1,5-a]pyrimidin-2-yl)methyl)pyridin-4-amine (30 mg, 0.087 mmol), (1S,2S)-2-(3-chlorophenyl)cyclopropane-1-carboxamide (17 mg, 0.087 mmol) and Cs₂CO₃ (85 mg, 0.26 mmol) in 1,4-dioxane (3 mL) was added Pd₂(dba)₃ (8 mg, 0.009 mmol) and Xantphos (10 mg, 0.017 mmol). The mixture was stirred at 90° C. for 16 h under N₂. After cooled to room temperature, the reaction mixture was filtered through Celite® and the filter cake was washed with DCM/MeOH (10/1, 20 mL). The filtrate was concentrated in vacuo to give the crude, which was purified by preparative TLC (eluent: DCM/MeOH=10/1) to give (1S,2S)-2-(3-chlorophenyl)-N-(4-(((6-cyclopropyl-[1,2,4]triazolo[1,5-a]pyrimidin-2-yl)methyl)amino)pyridin-2-yl)cyclopropane-1-carboxamide (12.2 mg, 31%) as a yellow solid. ESI-MS [M+H]⁺: 460.2. ¹H NMR (400 MHz, DMSO) δ 10.36 (s, 1H), 9.13 (d, J=2.4 Hz, 1H), 8.74 (d, J=2.4 Hz, 1H), 7.76 (d, J=5.8 Hz, 1H), 7.42 (s, 1H), 7.33-7.21 (m, 4H), 7.14 (d, J=7.6 Hz, 1H), 6.38-6.37 (m, 1H), 4.51 (d, J=6.1 Hz, 2H), 2.37-2.31 (m, 2H), 2.07-2.04 (m, 1H), 1.44-1.43 (m, 1H), 1.35-1.33 (m, 1H), 1.05-0.99 (m, 2H), 0.91-0.87 (m, 2H).

### Example 32

### Synthesis of (1S,2S)-2-(3-chlorophenyl)-N-(6-(((6-cyclopropyl-[1,2,4]triazolo[1,5-a]pyrimidin-2-yl)methyl)amino)pyrimidin-4-cyclopropane-1-carboxamide (1-32)

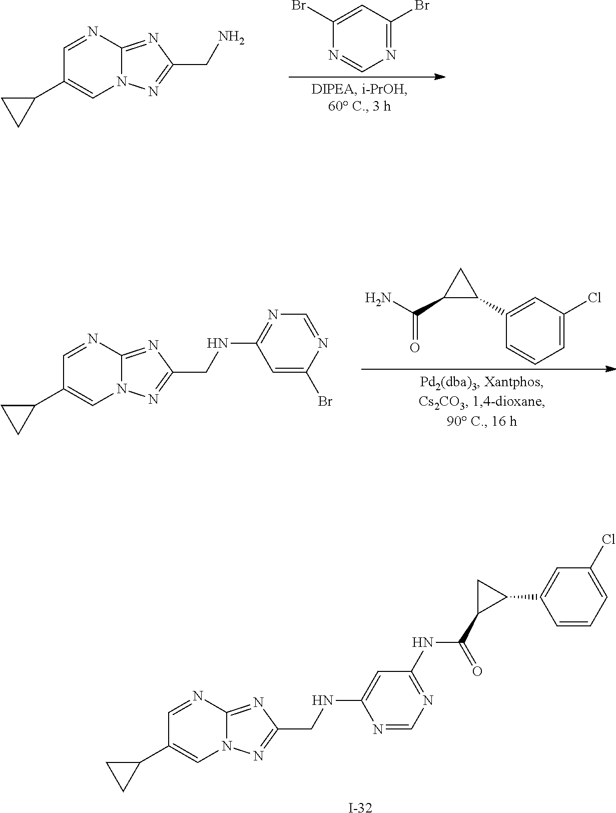 [SMILES: NCc1nc2ncc(C3CC3)cn2n1 ; Brc1cc(Br)ncn1 ; Brc1cc(NCc2nc3ncc(C4CC4)cn3n2)ncn1 ; NC(=O)[C@H]1C[C@@H]1c1cccc(Cl)c1 ; O=C(Nc1cc(NCc2nc3ncc(C4CC4)cn3n2)ncn1)[C@H]1C[C@@H]1c1cccc(Cl)c1 ; "I-32"]

Synthesis of 6-bromo-N-((6-cyclopropyl-[1,2,4]triazolo[1,5-a]pyrimidin-2-yl)methyl)pyrimidin-4-amine. A mixture of (6-cyclopropyl-[1,2,4]triazolo[1,5-a]pyrimidin-2-yl)methanamine (60 mg, 0.32 mmol), 4,6-dibromopyrimidine (76 mg, 0.32 mmol) and DIPEA (206 mg, 1.6 mmol) in i-PrOH (2 mL) was stirred at 60° C. for 3 h. The mixture was concentrated to give the crude product, which was purified by preparative TLC (eluent: DCM/MeOH=10/1) to give 6-bromo-N-((6-cyclopropyl-[1,2,4]triazolo[1,5-a]pyrimidin-2-yl)methyl)pyrimidin-4-amine (30 mg, 27%) as a white solid. ESI-MS [M+H]⁺: 346.1.

Synthesis of (1S,2S)-2-(3-chlorophenyl)-N-(6-(((6-cyclopropyl-[1,2,4]triazolo[1,5-a]pyrimidin-2-yl)methyl)amino)pyrimidin-4-yl)cyclopropane-1-carboxamide. To a mixture of 6-bromo-N-((6-cyclopropyl-[1,2,4]triazolo[1,5-a]pyrimidin-2-yl)methyl)pyrimidin-4-amine (30 mg, 0.087 mmol), (1S,2S)-2-(3-chlorophenyl)cyclopropane-1-carboxamide (17 mg, 0.087 mmol) and Cs₂CO₃ (85 mg, 0.26 mmol) in 1,4-dioxane (3 mL) was added Pd₂(dba)₃ (8 mg, 0.009 mmol) and Xantphos (10 mg, 0.017 mmol). The mixture was stirred at 90° C. for 16 h under N₂. The reaction mixture was cooled to room temperature and filtered through Celite®. The filter cake was washed with DCM/MeOH (10/1, 20 mL). The filtrate was concentrated in vacuo to give the crude, which was purified by preparative TLC (eluent: DCM/MeOH=15/1) to give (1S,2S)-2-(3-chlorophenyl)-N-(6-(((6-cyclopropyl-[1,2,4]triazolo[1,5-a]pyrimidin-2-yl)methyl)amino)pyrimidin-4-yl)cyclopropane-1-carboxamide (18 mg, 45%) as a yellow solid. ESI-MS [M+H]⁺: 461.2. ¹H NMR (400 MHz, DMSO) δ 10.61 (s, 1H), 9.11 (d, J=2.3 Hz, 1H), 8.73 (d, J=2.4 Hz, 1H), 8.16 (d, J=0.8 Hz, 1H), 8.04 (s, 1H), 7.34-7.30 (m, 2H), 7.27-7.24 (m, 2H), 7.16-7.14 (m, 1H), 4.74 (s, 2H), 2.42-2.34 (m, 2H), 2.11-2.04 (s, 1H), 1.50-1.45 (m, 1H), 1.44-1.39 (m, 1H), 1.04-0.99 (m, 2H), 0.90-0.86 (m, 2H).

### Example 33

### Synthesis of (1S,2S)-2-(3-chlorophenyl)-N-(4-(((3-cyclopropylquinolin-6-yl)methyl)amino)pyridin-2-yl)cyclopropane-1-carboxamide (I-33)

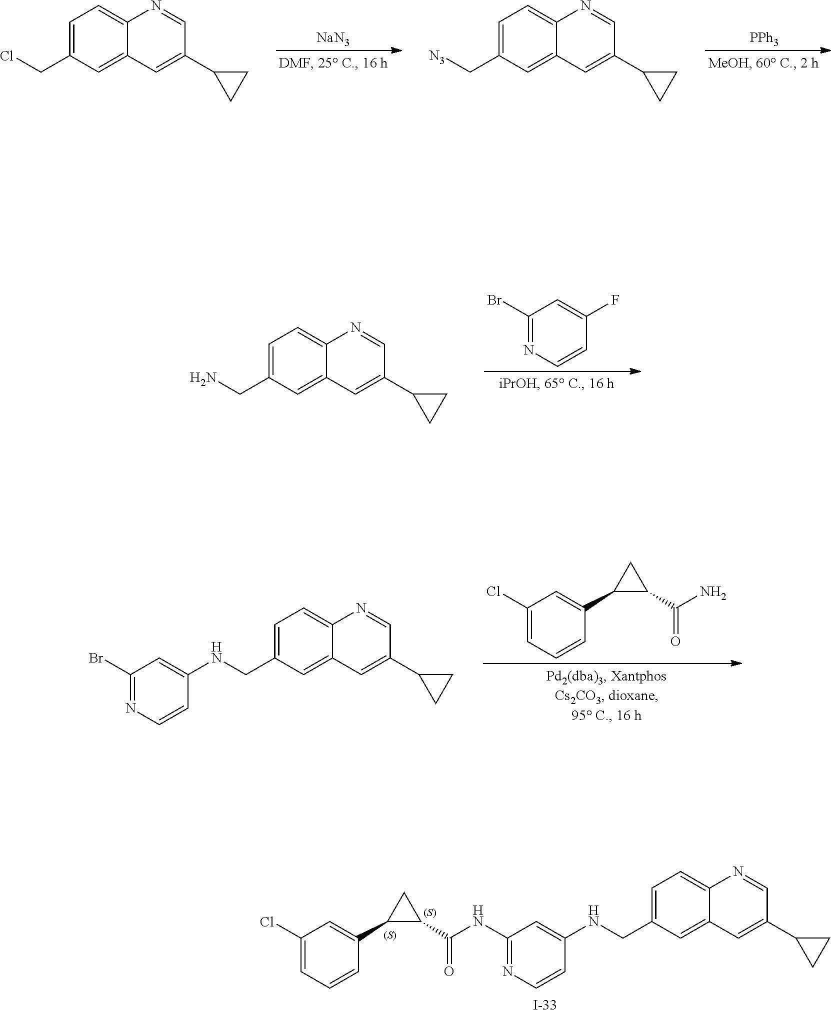

Synthesis of 6-(azidomethyl)-3-cyclopropylquinoline. To a mixture of 6-(chloromethyl)-3-cyclopropylquinoline (490 mg, 2.26 mmol) in DMF (10 mL) was added NaN₃ (536 mg, 8.25 mmol) slowly at 0° C. under N₂ atmosphere. The reaction mixture was then warmed to room temperature and stirred for 16 h. The reaction mixture was diluted with H₂O (50 mL) and extracted with EtOAc (50 mL×3). The combined organics were washed with brine (50 mL), dried over Na₂SO₄, and concentrated in vacuo. The residue was purified by column chromatography on silica gel, eluting with 0˜10% MeOH in DCM to give 6-(azidomethyl)-3-cyclopropylquinoline (320 mg, 63% yield) as a yellow oil. ESI-MS [M+H]⁺: 225.1.

Synthesis of (3-cyclopropylquinolin-6-yl)methanamine. To a mixture of 6-(azidomethyl)-3-cyclopropylquinoline (320 mg, 1.43 mmol) in MeOH (15 mL) was added PPh₃ (561 mg, 2.14 mmol). The reaction mixture was stirred at 60° C. for 2 h. The mixture was cooled to room temperature and concentrated in vacuo. The mixture was diluted with EtOAc (20 mL), treated with HCl (1 N in H₂O, 20 mL). The aqueous phase was washed with EtOAc (20 mL×2) and then concentrated in vacuo to give ((3-cyclopropylquinolin-6-yl)methanamine hydrochloride (300 mg, 90% yield) as a colorless oil. ESI-MS [M+H]⁺: 199.2.

Synthesis of 2-bromo-N-((3-cyclopropylquinolin-6-yl)methyl)pyridin-4-amine. A solution of ((3-cyclopropylquinolin-6-yl)methanamine hydrochloride (150 mg, 0.64 mmol), DIPEA (247 mg, 1.91 mL) and 2-bromo-4-fluoropyridine (133 mg, 0.76 mmol) in iPrOH (10 mL ) was stirred at 65° C. for 16 h under a N₂ atmosphere. After cooling to room temperature, the reaction was quenched with water (50 mL) and extracted with EtOAc (40 mL×3). The combined organic layers were washed with brine (30 mL), dried over Na₂SO₄, concentrated in vacuo to give the crude, which was purified by column chromatography on silica gel, eluting with 0˜10% MeOH in DCM to give 2-bromo-N-((3-cyclopropylquinolin-6-yl)methyl)pyridin-4-amine (50 mg, 22% yield) as a colorless oil. ESI-MS [M+H]⁺: 354.1.

Synthesis of (1S,2S)-2-(3-chlorophenyl)-N-(4-(((3-cyclopropylquinolin-6-yl)methyl)amino)pyridin-2-yl)cyclopropane-1-carboxamide. A solution of 2-bromo-N-((3-cyclopropylquinolin-6-yl)methyl)pyridin-4-amine (50 mg, 0.14 mmol), (1S,2S)-2-(3-chlorophenyl)cyclopropane-1-carboxamide (33 mg, 0.17 mmol), Pd₂(dba)₃ (18 mg, 0.02 mmol), Xantphos (23 mg, 0.04 mmol) and Cs₂CO₃ (130 mg, 0.4 mmol) in dioxane (10 mL ) was stirred at 95° C. for 16 h under a N₂ atmosphere. The mixture was cooled to room temperature and concentrated in vacuo. The residue was diluted with H₂O (40 mL) and extracted with EtOAc (30 mL×2). The combined organics were washed with brine (20 mL), dried over Na₂SO₄, and concentrated in vacuo. The residue was purified by column chromatography on silica gel, eluting with 0˜10% MeOH in DCM to give (1S,2S)-2-(3-chlorophenyl)-N-(4-(((3-cyclopropylquinolin-6-yl)methyl)amino)pyridin-2-yl)cyclopropane-1-carboxamide (11 mg, 17% yield). ESI-MS [M+H]⁺: 469.2. ¹H NMR (400 MHz, DMSO) δ 10.31 (s, 1H), 8.71 (d, J=2.2 Hz, 1H), 7.94-7.88 (m, 2H), 7.75-7.73 (m, 2H), 7.63-7.60 (m, 1H), 7.45 (s, 1H), 7.37-7.21 (m, 4H), 7.13 (d, J=7.7 Hz, 1H), 6.28 (d, J=3.8 Hz, 1H), 4.54-4.39 (m, 2H), 2.40-2.27 (m, 2H), 2.16-2.08 (m, 1H), 1.46-1.39 (m, 1H), 1.36-1.28 (m, 1H), 1.10-1.00 (m, 2H), 0.94-0.80 (m, 2H).

### Example 34

### Synthesis of (1S,2S)-2-(3-chlorophenyl)-N-(4-(((6-cyclopropylquinolin-3-yl)methyl)amino)pyridin-2-yl)cyclopropane-1-carboxamide (1-34)

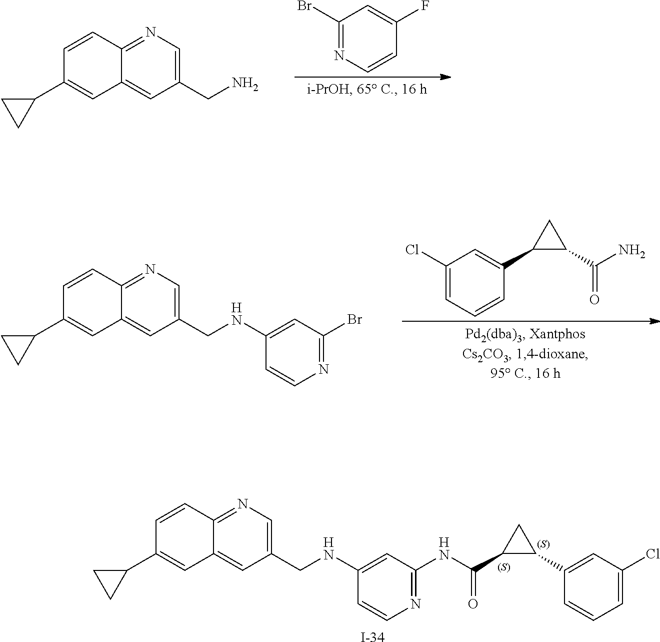

Synthesis of 2-bromo-N-((6-cyclopropylquinolin-3-yl)methyl)pyridin-4-amine. A solution of (6-cyclopropylquinolin-3-yl)methanamine (200 mg, 1.01 mmol, obtained from the corresponding hydrochloric acid salt), 2-bromo-4-fluoropyridine (449 mg, 2.55 mmol) and DIPEA (329 mg, 2.55 mmol) in iPrOH (10 mL ) was stirred at 60° C. for 16 h N₂ atmosphere. The mixture was cooled to room temperature and concentrated in vacuo. The residue was purified by column chromatography on silica gel, eluting with 0˜10% MeOH in DCM to give 2-bromo-N-((6-cyclopropylquinolin-3-yl)methyl)pyridin-4-amine (120 mg, 34% yield) as colorless oil. ESI-MS [M+H]⁺: 354.1.

Synthesis of (1S,2S)-2-(3-chlorophenyl)-N-(4-(((6-cyclopropylquinolin-3-yl)methyl)amino)pyridin-2-yl)cyclopropane-1-carboxamide. A solution of 2-bromo-N-((6-cyclopropylquinolin-3-yl)methyl)pyridin-4-amine (120 mg, 0.33 mmol), (1S,2S)-2-(3-chlorophenyl)cyclopropane-1-carboxamide (78 mg, 0.4 mmol), Pd₂(dba)₃ (46 mg, 0.05 mmol), Xantphos (174 mg, 0.3 mmol) and Cs₂CO₃ (130 mg, 0.4 mmol) in 1,4-dioxane (10 mL) was stirred at 95° C. for 16 h under N₂ atmosphere. The mixture was cooled to room temperature and concentrated in vacuo. The residue was diluted with H₂O (40 mL) and extracted with EtOAc (20 mL×2). The combined organics was washed with brine (20 mL), dried over Na₂SO₄, and concentrated in vacuo. The residue was purified by prep-TLC, eluting with 10% MeOH in DCM to give (1S,2S)-2-(3-chlorophenyl)-N-(4-(((6-cyclopropylquinolin-3-yl)methyl)amino)pyridin-2-yl) cyclopropane-1-carboxamide (14 mg, 9% yield) as an off-white solid. ESI-MS [M+H]⁺: 469.2. ¹H NMR (400 MHz, DMSO) δ 10.33 (s, 1H), 8.79 (d, J=2.2 Hz, 1H), 8.08 (d, J=1.3 Hz, 1H), 7.88 (d, J=8.7 Hz, 1H), 7.76 (d, J=5.8 Hz, 1H), 7.61 (d, J=1.9 Hz, 1H), 7.50-7.41 (m, 2H), 7.34-7.27 (m, 2H), 7.27-7.19 (m, 2H), 7.16-7.10 (m, 1H), 6.34-6.28 (m, 1H), 4.49 (d, J=5.8 Hz, 2H), 2.39-2.31 (m, 2H), 2.15-2.06 (m, 1H), 1.47-1.40 (m, 1H), 1.37-1.30 (m, 1H), 1.07-1.00 (m, 2H), 0.84-0.78 (m, 2H).

### Example 35

### Synthesis of (1S,2S)-2-(3-chlorophenyl)-N-(2-((6-cyclopropylimidazo[1,2-b]pyridazin-2-yl)methyl)-2H-pyrazolo[4,3-c]pyridin-4-yl)cyclopropane-1-carboxamide (I-35)

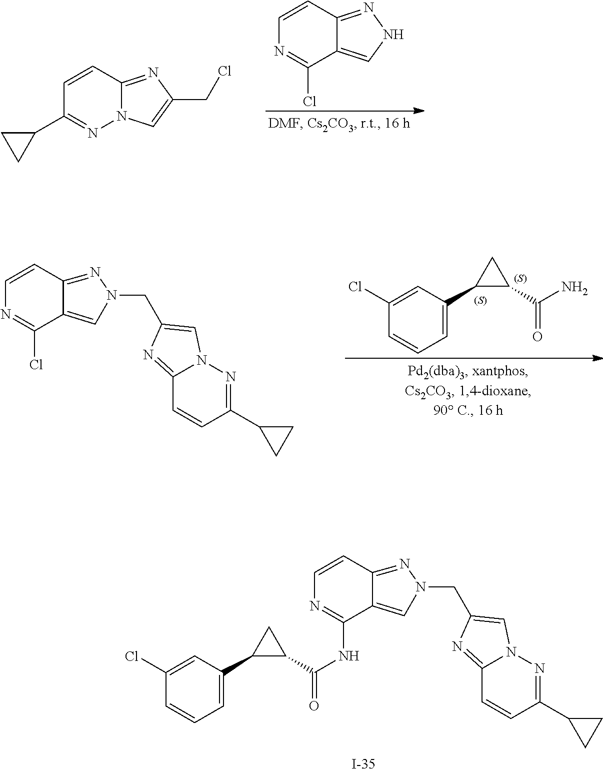

Synthesis of 4-chloro-2-((6-cyclopropylimidazo[1,2-b]pyridazin-2-yl)methyl)-2H-pyrazolo[4,3-c]pyridine. A mixture of 2-(chloromethyl)-6-cyclopropylimidazo[1,2-b]pyridazine (1 g, 4.82 mmol), 4-chloro-2H-pyrazolo[4,3-c]pyridine (740 mg, 4.82 mmol) and Cs₂CO₃ (4.7 g, 14.46 mmol) in DMF (30 mL) was stirred at room temperature for 16 h under N₂. The reaction was diluted with H₂O (60 mL), extracted with EtOAc (60 mL×3). The combined organic layers washed with brine (70 mL), dried over anhydrous Na₂SO₄, concentrated in vacuo to give the crude, which was purified by silica gel chromatography eluting with a gradient of 0-5% MeOH in DCM and followed by preparative HPLC to give 4-chloro-2-((6-cyclopropylimidazo[1,2-b]pyridazin-2-yl)methyl)-2H-pyrazolo[4,3-c]pyridine (300 mg, 19%) as a white solid. ESI-MS [M+H]⁺: 325.1.

Synthesis of (1S,2S)-2-(3-chlorophenyl)-N-(2-((6-cyclopropylimidazo[1,2-b]pyridazin-2-yl)methyl)-2H-pyrazolo[4,3-c]pyridin-4-yl)cyclopropane-1-carboxamide. To a mixture of give 4-chloro-2-((6-cyclopropylimidazo[1,2-b]pyridazin-2-yl)methyl)-2H-pyrazolo[4,3-c]pyridine (100 mg, 0.31 mmol) in 1,4-dioxane (10 mL) was added (1S,2S)-2-(3-chlorophenyl)cyclopropane-1-carboxamide (80 mg, 0.41 mmol), Pd₂(dba)₃ (57 mg, 0.062 mmol), Xantphos (36 mg, 0.062 mmol), Cs₂CO₃ (303 mg, 0.93 mmol). The reaction mixture was stirred at 90° C. for 16 h under N₂. After cooling to room temperature, the reaction mixture was filtered through Celite® and the filter cake was washed with DCM/MeOH (10/1, 40 mL). The filtrate was concentrated in vacuo to give the crude, which was purified by silica gel column chromatography eluting with a gradient of 0-10% MeOH in DCM to give (1S,2S)-2-(3-chlorophenyl)-N-(2-((6-cyclopropylimidazo[1,2-b]pyridazin-2-yl)methyl)-2H-pyrazolo[4,3-c]pyridin-4-yl)cyclopropane-1-carboxamide (7 mg, 5%) as a white solid. ESI-MS [M+H]⁺: 484.2. ¹H NMR (400 MHz, DMSO) δ 10.98 (s, 1H), 8.82 (s, 1H), 8.18 (s, 1H), 7.92 (d, J=9.5 Hz, 1H), 7.85 (d, J=6.2 Hz, 1H), 7.35-7.18 (m, 5H), 7.10 (d, J=9.5 Hz, 1H), 5.77 (s, 2H), 2.49-2.45 (m, 1H), 2.21-2.14 (m, 1H), 2.02-1.95 (m, 1H), 1.58-1.53 (m, 1H), 1.49-1.45 (m, 1H), 1.09-1.04 (m, 2H), 0.99-0.95 (m, 2H).

### Example 36

### Synthesis of (1S,2S)-2-(3-chlorophenyl)-N-(4-(((6-cyclopropyl-5-oxo-5,6-dihydroimidazo[1,2-c]pyrimidin-2-yl)methyl)amino)pyridin-2-yl)cyclopropane-1-carboxamide (I-36)

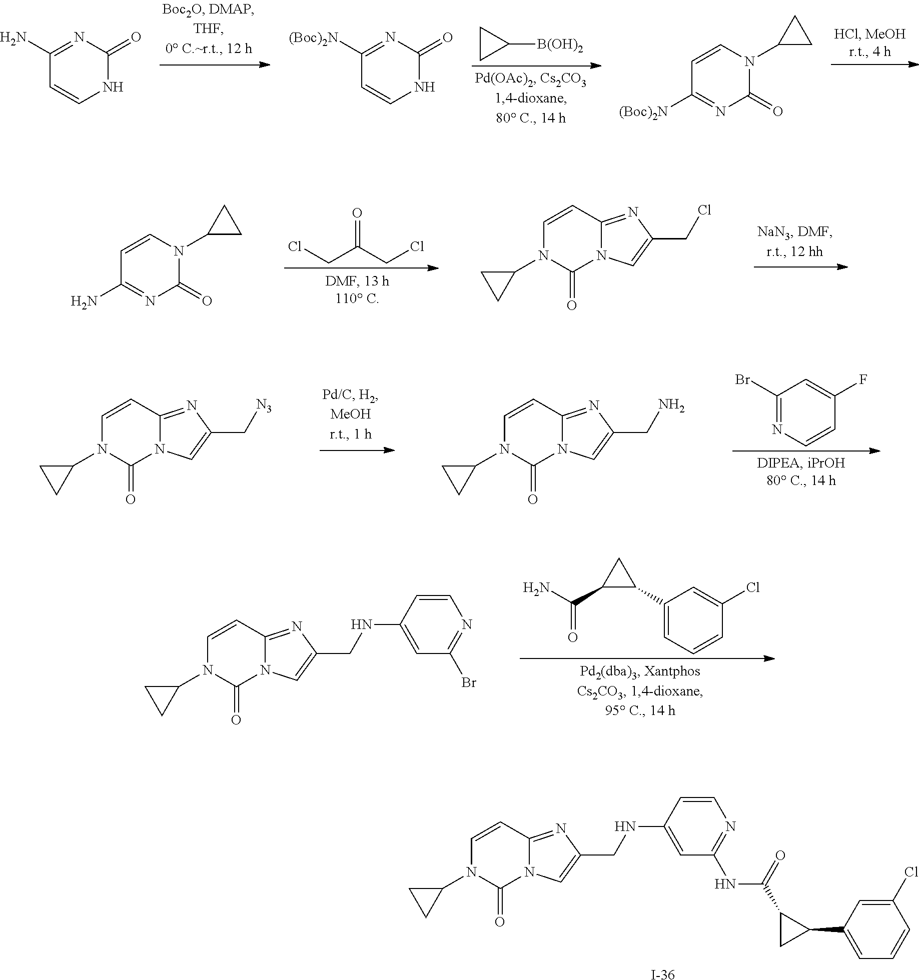

Synthesis of tert-butyl (tert-butoxycarbonyl)(2-oxo-1,2-dihydropyrimidin-4-yl)carbamate. To a solution of 4-aminopyrimidin-2 (1H)-one (3.33 g, 30 mmol) and DMAP (363 mg, 3 mmol) in THF (50 mL) was added Boc₂O (13.1 g, 60 mmol) at 0° C. The resulting reaction solution was stirred at room temperature for 12 h. The reaction was diluted with water (100 mL) then extracted with EtOAc (80 mL×3). The combined organic layers were washed with brine (80 mL), dried over anhydrous Na₂SO₄ and concentrated in vacuo to give the crude, which was purified with silica gel column chromatography eluting with a gradient of 0-30% EtOAc in PE to give tert-butyl (tert-butoxycarbonyl)(2-oxo-1,2-dihydropyrimidin-4-yl)carbamate (6.5 g, 70%) as a white solid. ESI-MS [M+1]⁺: 312.2.

Synthesis of tert-butyl (tert-butoxycarbonyl)(1-cyclopropyl-2-oxo-1,2-dihydropyrimidin-4-yl)carbamate. A mixture of tert-butyl (tert-butoxycarbonyl)(2-oxo-1,2-dihydropyrimidin-4-yl)carbamate (6.5 g, 20.8 mmol), cyclopropylboronic acid (3.6 g, 41.6 mmol), Pd(OAc)₂ (448 mg, 2 mmol) and Cs₂CO₃ (20.3 g, 62.4 mmol) in 1,4-dioxane (50 mL) was stirred at 80° C. for 14 h. The reaction was cooled to room temperature, diluted with H₂O (80 mL) then extracted with EtOAc (80 mL×3). The combined organic layers were washed with brine (80 mL), dried over Na₂SO₄ then concentrated in vacuo to give the crude, which was purified with silica gel column chromatography eluting with a gradient of 0-25% EtOAc in PE to give tert-butyl (tert-butoxycarbonyl)(1-cyclopropyl-2-oxo-1,2-dihydropyrimidin-4-yl)carbamate (2.3 g, 31%) as a yellow solid. ESI-MS [M+1]⁺: 352.2.

Synthesis of 4-amino-1-cyclopropylpyrimidin-2 (1H)-one. To a solution of tert-butyl (tert-butoxycarbonyl)(1-cyclopropyl-2-oxo-1,2-dihydropyrimidin-4-yl)carbamate (2.3 g, 6.5 mmol) in MeOH (30 mL) was added HCl (4M solution in MeOH, 5 mL). The resulting reaction was stirred at room temperature for 4 h. The reaction was concentrated in vacuo to give 4-amino-1-cyclopropylpyrimidin-2 (1H)-one (2.4 g, crude), which was used without further purification. ESI-MS [M+1]⁺: 152.2

Synthesis of 2-(chloromethyl)-6-cyclopropylimidazo[1,2-c]pyrimidin-5 (6H)-one. A mixture of 4-amino-1-cyclopropylpyrimidin-2 (1H)-one (2.4 g, crude) and 1,3-dichloropropan-2-one (2.5 g, 19.8 mmol) in DMF (40 mL) was stirred at 110° C. for 13 h. After cooling to room temperature, the reaction was quenched with NaHCO₃ (sat. aq., 60 mL) then extracted with EtOAc (60 mL×3). The combined organic layers was washed with brine (80 mL), dried over anhydrous Na₂SO₄ and concentrated in vacuo to give the crude, which was purified with silica gel column chromatography eluting with a gradient of 0-5% MeOH in DCM to give 2-(chloromethyl)-6-cyclopropylimidazo[1,2-c]pyrimidin-5 (6H)-one (500 mg, 34% over 2 steps) as a yellow solid. ESI-MS [M+1]⁺: 224.1.

Synthesis of 2-(azidomethyl)-6-cyclopropylimidazo[1,2-c]pyrimidin-5 (6H)-one. A 2-(chloromethyl)-6-cyclopropylimidazo[1,2-c]pyrimidin-5 (6H)-one (500 mg, 2.2 mmol) and NaN₃ (286 mg, 4.4 mmol) in DMF (20 mL) was stirred at room temperature for 12 h. The reaction was diluted with H₂O (30 mL) and extracted with EtOAc (30 mL×3). The combined organic layers were washed with brine (30 mL), dried over Na₂SO₄ then concentrated in vacuo to give 2-(azidomethyl)-6-cyclopropylimidazo[1,2-c]pyrimidin-5 (6H)-one (550 mg, crude), which was used without further purification. ESI-MS [M+1]⁺: 231.2

Synthesis of 2-(aminomethyl)-6-cyclopropylimidazo[1,2-c]pyrimidin-5 (6H)-one. A mixture of 2-(azidomethyl)-6-cyclopropylimidazo[1,2-c]pyrimidin-5 (6H)-one (550 mg, crude from previous step) and Pd/C (100 mg) in MeOH (30 mL) was stirred at room temperature under a H₂ atmosphere for 1 h. The reaction was filtered through Celite®, which was washed with MeOH (50 mL). The filtrate was concentrated in vacuo to give the crude, which was purified with silica gel column chromatography eluting with a gradient of 0-10% MeOH in DCM to give 2-(aminomethyl)-6-cyclopropylimidazo[1,2-c]pyrimidin-5 (6H)-one (350 mg, 78% over two steps) as a yellow solid. ESI-MS [M+1]⁺: 205.2.

Synthesis of 2-(aminomethyl)-6-cyclopropylimidazo[1,2-c]pyrimidin-5 (6H)-one. A solution of 2-(aminomethyl)-6-cyclopropylimidazo[1,2-c]pyrimidin-5 (6H)-one (250 mg, 1.2 mmol), 2-bromo-4-fluoropyridine (281 mg, 1.6 mmol) and DIPEA (787 mg, 6.0 mmol) in iPrOH (15 mL) was stirred at 80° C. for 14 h. The reaction was concentrated in vacuo to give the crude, which was purified with preparative TLC (eluent: DCM/MeOH=10/1) to give 2-(((2-bromopyridin-4-yl)amino)methyl)-6-cyclopropylimidazo[1,2-c]pyrimidin-5 (6H)-one (80 mg, 18%) as a yellow solid. ESI-MS [M+1]⁺: 360.0.

Synthesis of (1S,2S)-2-(3-chlorophenyl)-N-(4-(((6-cyclopropyl-5-oxo-5,6-dihydroimidazo[1,2-c]pyrimidin-2-yl)methyl)amino)pyridin-2-yl)cyclopropane-1-carboxamide. A mixture of 2-(((2-bromopyridin-4-yl)amino)methyl)-6-cyclopropylimidazo[1,2-c]pyrimidin-5 (6H)-one (80 mg, 0.22 mmol), (1S,2S)-2-(3-chlorophenyl)cyclopropane-1-carboxamide (51 mg, 0.26 mmol), Pd₂(dba)₃ (30 mg, 0.033 mmol), Xantphos (25 mg, 0.044 mmol) and Cs₂CO₃ (215 mg, 0.66 mmol) in 1,4-dioxane (10 mL) was stirred at 95° C. for 14 h. The reaction was cooled to room temperature, diluted with H₂O (20 mL) then extracted with EtOAc (30 mL×3). The combined organic layers were washed with brine (30 mL), dried over anhydrous Na₂SO₄ and concentrated in vacuo to give the crude, which was purified with preparative TLC (eluent: DCM/MeOH=10/1) to give (1S,2S)-2-(3-chlorophenyl)-N-(4-(((6-cyclopropyl-5-oxo-5,6-dihydroimidazo[1,2-c]pyrimidin-2-yl)methyl)amino)pyridin-2-yl)cyclopropane-1-carboxamide (4 mg, 4%) as a white solid. ESI-MS [M+1]⁺: 475.2. ¹H NMR (400 MHz, DMSO) δ 10.61 (s, 1H), 8.19 (s, 1H), 7.94 (s, 1H), 7.78 (s, 1H), 7.70 (s, 1H), 7.60 (d, J=6.4 Hz, 1H), 7.34-7.25 (m, 3H), 7.15 (d, J=7.6 Hz, 1H), 6.77 (d, J=6.4 Hz, 1H), 4.56 (s, 2H), 2.89-2.85 (m, 1H), 2.43-2.25 (m, J=12.6 Hz, 2H), 1.54-1.43 (m, J=4.1 Hz, 1H), 1.44-1.31 (m, 1H), 0.85-0.73 (m, 2H), 0.60-0.56 (m, 2H).

### Examples 37 and 38

### Synthesis of (1S,2S)-2-(3-chlorophenyl)-N-(6-(((5-isopropoxyimidazo[1,2-c]pyrimidin-2-yl)methyl)amino)pyrimidin-4-yl)cyclopropane-1-carboxamide (I-37), and (1S,2S)-2-(3-chlorophenyl)-N-(6-(((6-isopropyl-5-oxo-5,6-dihydroimidazo[1,2-c]pyrimidin-2-yl)methyl)amino)pyrimidin-4-yl)cyclopropane-1-carboxamide (I-38N

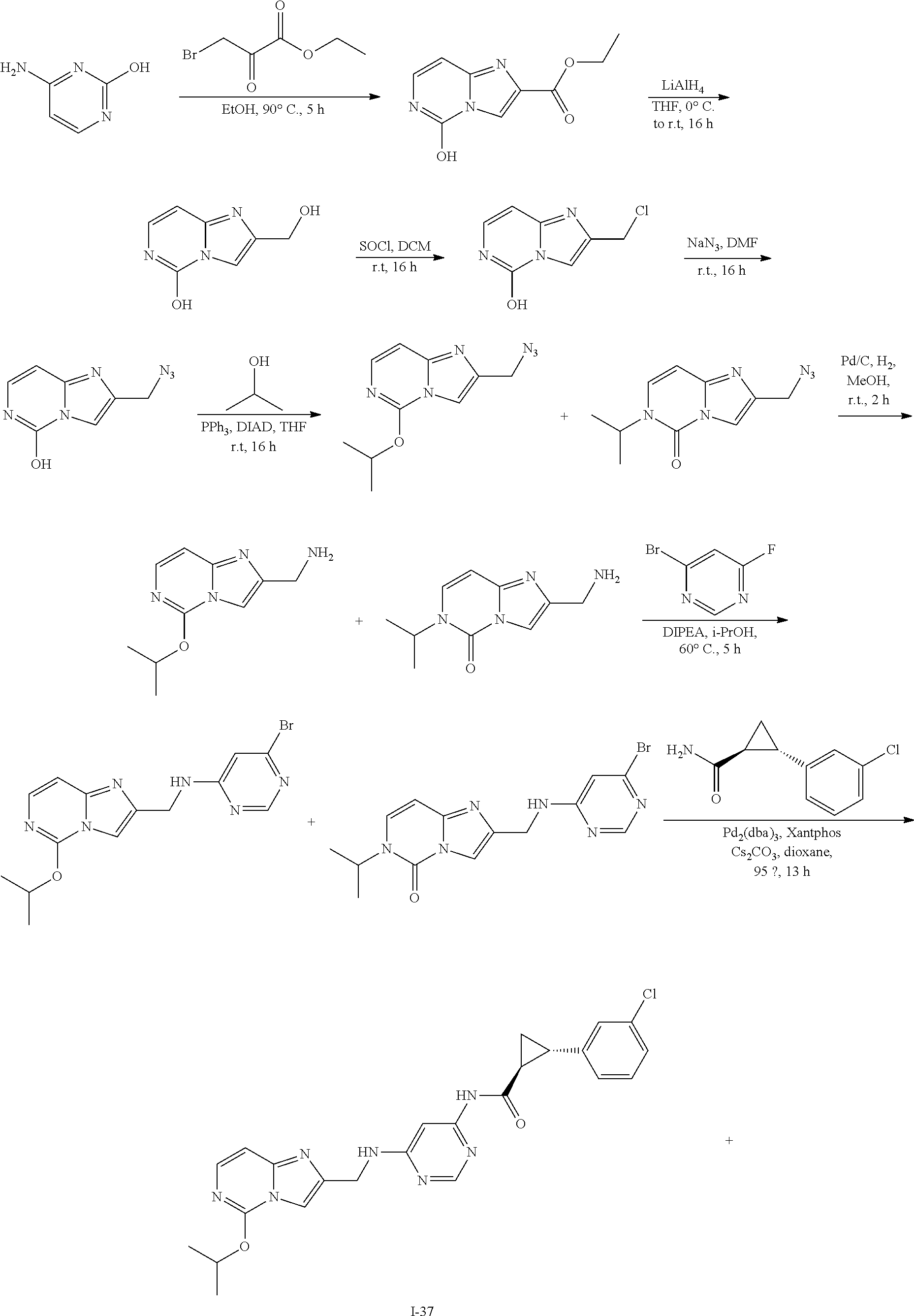

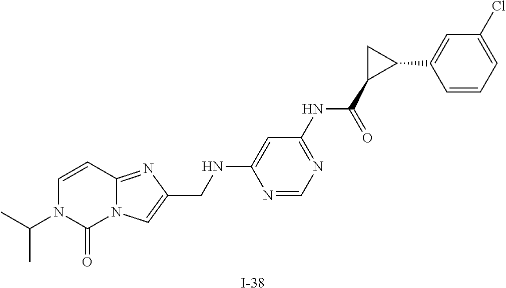 [SMILES: CC(C)n1ccc2nc(CNc3cc(NC(=O)[C@H]4C[C@@H]4c4cccc(Cl)c4)ncn3)cn2c1=O ; "I-38"]

Synthesis of ethyl d-hydroxyimidazo[1,2-c]pyrimidine-2-carboxylate. A mixture of 4-aminopyrimidin-2-ol (1.1 g, 10.0 mmol) and ethyl 3-bromo-2-oxopropanoate (1.9 g, 10.0 mmol) in EtOH (20 mL) was stirred at 90° C. for 5 h. The reaction mixture was concentrated in vacuo to give the crude, which was purified by column chromatography (eluent: DCM/MeOH=20/1) to give ethyl 5-hydroxyimidazo[1,2-c]pyrimidine-2-carboxylate (500 mg, 24%) as a white solid. ESI-MS [M+H]: 208.1.

Synthesis of 2-(hydroxymethyl)imidazo[1,2-c]pyrimidin-5-ol. To a solution of ethyl 5-hydroxyimidazo[1,2-c]pyrimidine-2-carboxylate (500 mg, 2.4 mmol) in THF (20 mL) was added LiAH₄ (182 mg, 4.8 mmol) at 0° C. The reaction mixture was stirred at room temperature for 16 h under N₂. The reaction was quenched with water (50 mL) and extracted with EtOAc/MeOH (10/1, 30 mL×3). The combined organic layers were washed with brine (30 mL), dried over anhydrous Na₂SO₄, and concentrated in vacuo to give the crude product, which was purified by column chromatography (eluent: DCM/MeOH=10/1) to give 2-(hydroxymethyl)imidazo[1,2-c]pyrimidin-5-ol (200 mg, 51%) as a white solid. ESI-MS [M+H]⁺: 166.1.

Synthesis of 2-(chloromethyl)imidazo[1,2-c]pyrimidin-5-ol. To a solution of 2-(hydroxymethyl)imidazo [1,2-c]pyrimidin-5-ol (200 mg, 1.2 mmol) in DCM (10 mL) was added SOCl₂ (2.0 mL). The resulting mixture was stirred at room temperature for 16 h. The mixture was concentrated in vacuo to give 2-(chloromethyl)imidazo[1,2-c]pyrimidin-5-ol (210 mg crude) as a yellow solid, which was used into next step without further purification. ESI-MS [M+H]⁺: 184.0.

Synthesis of 2-(azidomethyl)imidazo[1,2-c]pyrimidin-5-ol. To a solution of 2-(chloromethyl)imidazo[1,2-c]pyrimidin-5-ol (210 mg, crude) in DMF (10 mL) was added NaN₃ (195 mg, 3 mmol). The resulting mixture was stirred at room temperature for 16 h. The reaction mixture was diluted with H₂O (50 mL) and extracted with EtOAc (50 mL×3). The combined organics were washed with brine (30 mL), dried over Na₂SO₄ and concentrated to give the crude, which was purified by preparative TLC (eluent: DCM/MeOH=15/1) to give 2-(azidomethyl)imidazo[1,2-c]pyrimidin-5-ol (120 mg, 53% over 2 steps) as a yellow solid. ESI-MS [M+H]⁺: 191.1.

Synthesis of 2-(azidomethyl)-5-isopropoxyimidazo[1,2-c]pyrimidine and 2-(azidomethyl)-6-isopropylimidazo[1,2-c]pyrimidin-5 (6H)-one. To a solution of 2-(azidomethyl)imidazo[1,2-c]pyrimidin-5-ol (120 mg, 0.63 mmol), propan-2-ol (565 mg, 9.4 mmol), PPh₃ (247 mg, 0.94 mmol) in THF (15 mL) was added DIAD (191 mg, 0.94 mmol). The resulting reaction was stirred at room temperature for 16 h under N₂ then was quenched with H₂O (30 mL) and then extracted with EtOAc (50 mL×3). The combined organics were washed with brine (30 mL), dried over Na₂SO₄, and concentrated in vacuo. The residue was purified by column chromatography on silica gel, eluting with 0˜10% MeOH in DCM to give a mixture of 2-(azidomethyl)-5-isopropoxyimidazo[1,2-c]pyrimidine and 2-(azidomethyl)-6-isopropylimidazo[1,2-c]pyrimidin-5 (6H)-one (100 mg, 68%) as a yellow solid. ESI-MS [M+H]⁺: 233.1.

Synthesis of (5-isopropoxyimidazo[1,2-c]pyrimidin-2-yl)methanamine and 2-(aminomethyl)-6-isopropylimidazo[1,2-c]pyrimidin-5 (6H)-one. A mixture of 2-(azidomethyl)-5-isopropoxyimidazo[1,2-c]pyrimidine (mixed with 2-(azidomethyl)-6-isopropylimidazo[1,2-c]pyrimidin-5 (6H)-one) (100 mg, 0.43 mmol) and Pd/C (30 mg) in MeOH (10 mL) was stirred at room temperature for 2 h under a H₂ atmosphere. The resulting mixture was filtered through Celite® and the filter cake was washed with DCM/MeOH (10/1, 30 mL). The filtrate was concentrated in vacuo to give a mixture of (5-isopropoxyimidazo[1,2-c]pyrimidin-2-yl)methanamine and 2-(aminomethyl)-6-isopropylimidazo[1,2-c]pyrimidin-5 (6H)-one (95 mg, crude) as a yellow solid. ESI-MS [M+H]⁺: 207.1.

Synthesis of 6-bromo-N-((5-isopropoxyimidazo[1,2-c]pyrimidin-2-yl)methyl)pyrimidin-4-amine and 2-(((6-bromopyrimidin-4-yl)amino)methyl)-6-isopropylimidazo[1,2-c]pyrimidin-5 (6H)-one. A solution of (5-isopropoxyimidazo[1,2-c]pyrimidin-2-yl)methanamine (as a mixture with 2-(aminomethyl)-6-isopropylimidazo[1,2-c]pyrimidin-5 (6H)-one) (95 mg crude), 4,6-dibromopyrimidine (256 mg, 1.1 mmol) and DIPEA (465 mg, 3.6 mmol) in iPrOH (10 mL) was stirred at 60° C. for 2 h. The mixture was cooled to room temperature and concentrated in vacuo. The residue was purified by column chromatography on silica gel, eluting with 0˜10% MeOH in DCM to give a mixture of 6-bromo-N-((5-isopropoxyimidazo[1,2-c]pyrimidin-2-yl)methyl)pyrimidin-4-amine and 2-(((6-bromopyrimidin-4-yl)amino)methyl)-6-isopropylimidazo[1,2-c]pyrimidin-5 (6H)-one (100 mg, 64% over 2 steps) as a yellow solid. ESI-MS [M+H]⁺: 363.0.

Synthesis of (1S,2S)-2-(3-chlorophenyl)-N-(6-(((5-isopropoxyimidazo[1,2-c]pyrimidin-2-yl)methyl)amino)pyrimidin-4-yl)cyclopropane-1-carboxamide, and (1S,2S)-2-(3-chlorophenyl)-N-(6-(((6-isopropyl-5-oxo-5,6-dihydroimidazo[1,2-c]pyrimidin-2-yl)methyl)amino)pyrimidin-4-yl)cyclopropane-1-carboxamide. A solution of 6-bromo-N-((5-isopropoxyimidazo[1,2-c]pyrimidin-2-yl)methyl)pyrimidin-4-amine (as a mixture with 2-(((6-bromopyrimidin-4-yl)amino)methyl)-6-isopropylimidazo[1,2-c]pyrimidin-5 (6H)-one (100 mg, 0.27 mmol), (1S,2S)-2-(3-chlorophenyl)cyclopropane-1-carboxamide (53 mg, 0.27 mmol), Pd₂(dba)₃ (49 mg, 0.054 mmol), Xantphos (31 mg, 0.054 mmol), and Cs₂CO₃ (264 mg, 0.81 mmol) in 1,4-dioxane (10 mL) was stirred at 95° C. for 2 h under a N₂ atmosphere. The mixture was cooled to room temperature and concentrated in vacuo. The residue was diluted with H₂O (20 mL) and extracted with EtOAc (50 mL×2). The combined organics were washed with brine (20 mL), dried over Na₂SO₄, and concentrated in vacuo. The residue was purified by preparative HPLC to give (1S,2S)-2-(3-chlorophenyl)-N-(6-(((5-isopropoxyimidazo[1,2-c]pyrimidin-2-yl)methyl)amino)pyrimidin-4-yl)cyclopropane-1-carboxamide (30 mg, 23%) and (1S,2S)-2-(3-chlorophenyl)-N-(6-(((6-isopropyl-5-oxo-5,6-dihydroimidazo[1,2-c]pyrimidin-2-yl)methyl)amino)pyrimidin-4-yl)cyclopropane-1-carboxamide (10 mg, 8%) as white solids.

(1S,2S)-2-(3-chlorophenyl)-N-(6-(((5-isopropoxyimidazo[1,2-c]pyrimidin-2-yl)methyl)amino)pyrimidin-4-yl)cyclopropane-1-carboxamide (I-37). ESI-MS [M+H]⁺: 464.3. ¹H NMR (400 MHz, DMSO) δ 10.54 (s, 1H), 8.12 (s, 1H), 7.80 (s, 1H), 7.58 (d, J=6.5 Hz, 1H), 7.41 (s, 1H), 7.25-7.17 (m, 4H), 7.07 (d, J=7.6 Hz, 1H), 7.03 (d, J=6.5 Hz, 1H), 5.40-5.34 (m, 1H), 4.52 (s, 2H), 2.34-2.27 (m, 2H), 1.41-1.37 (m, 1H), 1.33 (d, J=6.2 Hz, 6H), 1.31 (d, J=4.4 Hz, 1H).

(1S,2S)-2-(3-chlorophenyl)-N-(6-(((6-isopropyl-5-oxo-5,6-dihydroimidazo[1,2-c]pyrimidin-2-yl)methyl)amino)pyrimidin-4-yl)cyclopropane-1-carboxamide (I-38). ESI-MS [M+H]+: 464.3. ¹H NMR (400 MHz, DMSO) δ 10.61 (s, 1H), 8.20 (s, 1H), 7.84 (s, 1H), 7.57 (d, J=7.9 Hz, 1H), 7.53 (s, 1H), 7.32-7.25 (m, 4H), 7.16 (d, J=7.6 Hz, 1H), 6.64 (d, J=7.9 Hz, 1H), 4.94-4.88 (m, 1H), 4.51 (s, 2H), 2.43-2.36 (m, 2H), 1.50-1.46 (m, 1H), 1.43-1.39 (m, 1H), 1.35 (d, J=6.8 Hz, 6H).

### Example 39

### Synthesis of (1S,2S)-2-(3-chlorophenyl)-N-(6-(((6-cyclopropyl-8-(4-methylpiperazin-1-yl)imidazo[1,2-b]pyridazin-2-yl)methyl)amino)pyrimidin-4-yl)cyclopropane-1-carboxamide (I-39)

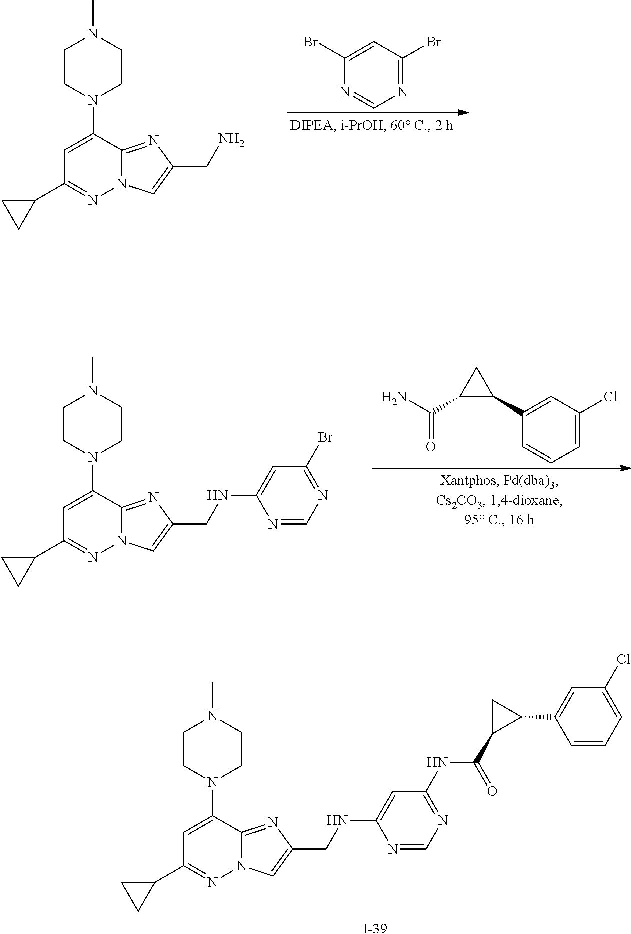 [SMILES: CN1CCN(c2cc(C3CC3)nn3cc(CN)nc23)CC1 ; Brc1cc(Br)ncn1 ; CN1CCN(c2cc(C3CC3)nn3cc(CNc4cc(Br)ncn4)nc23)CC1 ; NC(=O)[C@@H]1C[C@H]1c1cccc(Cl)c1 ; CN1CCN(c2cc(C3CC3)nn3cc(CNc4cc(NC(=O)[C@H]5C[C@@H]5c5cccc(Cl)c5)ncn4)nc23)CC1 ; "I-39"]

Synthesis of 6-bromo-N-((6-cyclopropyl-8-(4-methylpiperazin-1-yl)imidazo[1,2-b]pyridazin-2-yl)methyl)pyrimidin-4-amine. A mixture of (6-cyclopropyl-8-(4-methylpiperazin-1-yl)imidazo[1,2-b]pyridazin-2-yl)methanamine (120 mg, 0.42 mmol), 4,6-dibromopyrimidine (100 mg, 0.42 mmol) and DIPEA (163 mg, 1.26 mmol) in i-PrOH (5 mL) was stirred at 60° C. for 2 h. The reaction mixture was concentrated in vacuo to give the crude, which was purified by preparative TLC (eluent: MeOH/DCM=1/10) to give 6-bromo-N-((6-cyclopropyl-8-(4-methylpiperazin-1-yl)imidazo[1,2-b]pyridazin-2-yl)methyl)pyrimidin-4-amine (150 mg, 81%) as a yellow solid. ESI-MS [M+H]⁺: 443.2.

Synthesis of (1S,2S)-2-(3-chlorophenyl)-N-(6-(((6-cyclopropyl-8-(4-methylpiperazin-1-yl)imidazo[1,2-b]pyridazin-2-yl)methyl)amino)pyrimidin-4-yl)cyclopropane-1-carboxamide. To a mixture of 6-bromo-N-((6-cyclopropyl-8-(4-methylpiperazin-1-yl)imidazo[1,2-b]pyridazin-2-yl)methyl)pyrimidin-4-amine (80 mg, 0.18 mmol), (1S,2S)-2-(3-chlorophenyl)cyclopropane-1-carboxamide (35 mg, 0.18 mmol) and Cs₂CO₃ (176 mg, 0.54 mmol) in 1,4-dioxane (6 mL) was added Pd₂(dba)₃ (18 mg, 0.02 mmol) and Xantphos (12 mg, 0.02 mmol). The mixture was stirred at 95° C. for 16 h under N₂. The reaction mixture was cooled to room temperature and filtered through Celite®. The filter cake was washed with DCM/MeOH (10/1, 30 mL). The filtrate was concentrated in vacuo to give the crude, which was purified by preparative TLC (eluent: DCM/MeOH=10/1) to give (1S,2S)-2-(3-chlorophenyl)-N-(6-(((6-cyclopropyl-8-(4-methylpiperazin-1-yl)imidazo[1,2-b]pyridazin-2-yl)methyl)amino)pyrimidin-4-yl)cyclopropane-1-carboxamide (51 mg, 51%) as a white solid. ESI-MS [M+H]⁺: 558.2. ¹H NMR (400 MHz, DMSO) δ 10.57 (s, 1H), 8.17 (s, 1H), 7.78 (s, 1H), 7.62 (s, 1H), 7.30-7.25 (m, 2H), 7.24-7.21 (m, 2H), 7.12 (d, J=7.6 Hz, 1H), 6.12 (s, 1H), 4.48 (s, 2H), 3.89-3.80 (m, 4H), 2.44-2.40 (m, 4H), 2.40-2.31 (m, 2H), 2.20 (s, 3H), 1.97-1.93 (m, 1H), 1.49-1.33 (m, 2H), 0.91-0.86 (m, 4H).

### Example 40

### Synthesis of (1S,2S)-2-(3-chlorophenyl)-N-(4-(((6-cyclopropyl-8-(4-methylpiperazin-1-yl)imidazo[1,2-b]pyridazin-2-yl)methyl)amino)pyridin-2-yl)cyclopropane-1-carboxamide (I-40)

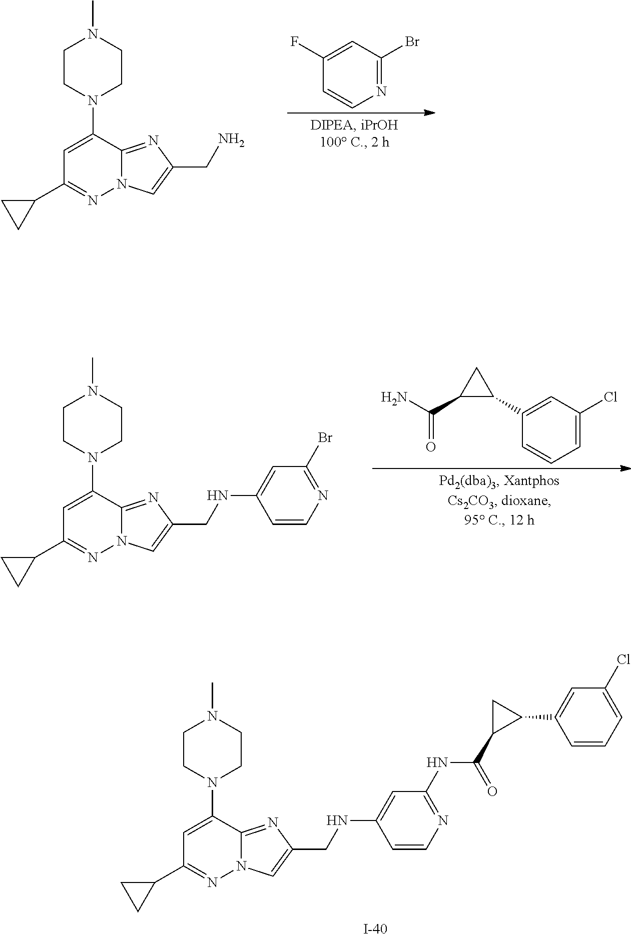 [SMILES: CN1CCN(c2cc(C3CC3)nn3cc(CN)nc23)CC1 ; Fc1ccnc(Br)c1 ; CN1CCN(c2cc(C3CC3)nn3cc(CNc4ccnc(Br)c4)nc23)CC1 ; NC(=O)[C@H]1C[C@@H]1c1cccc(Cl)c1 ; CN1CCN(c2cc(C3CC3)nn3cc(CNc4ccnc(NC(=O)[C@H]5C[C@@H]5c5cccc(Cl)c5)c4)nc23)CC1 ; "I-40"]

Synthesis of 2-bromo-N-((6-cyclopropyl-8-(4-methylpiperazin-1-yl)imidazo[1,2-b]pyridazin-2-yl)methyl)pyridin-4-amine. A mixture of (6-cyclopropyl-8-(4-methylpiperazin-1-yl)imidazo[1,2-b]pyridazin-2-yl)methanamine (150 mg, 0.52 mmol), 2-bromo-4-fluoropyridine (109 mg, 0.62 mmol) and DIPEA (201 mg, 1.56 mmol) in i-PrOH (3 mL) was stirred in a sealed tube. After degassing with N₂ for 1 min, the reaction was irradiated in a microwave reactor at 120° C. for 2 h. After cooling to room temperature, the reaction was quenched with NaHCO₃ (sat. aq., 30 mL) and extracted with EtOAc (30 mL×3). The combined organic layers were washed with brine (20 mL), dried over Na₂SO₄ then concentrated in vacuo to give the crude, which was purified by preparative TLC (eluent: DCM/MeOH=10/1) to give 2-bromo-N-((6-cyclopropyl-8-(4-methylpiperazin-1-yl)imidazo[1,2-b]pyridazin-2-yl)methyl)pyridin-4-amine as a yellow solid (60 mg, yield: 26%). ESI-MS [M+H]⁺: 442.1.

Synthesis of (1S,2S)-2-(3-chlorophenyl)-N-(4-(((6-cyclopropyl-8-(4-methylpiperazin-1-yl)imidazo[1,2-b]pyridazin-2-yl)methyl)amino)pyridin-2-yl)cyclopropane-1-carboxamide. A mixture of 2-bromo-N-((6-cyclopropyl-8-(4-methylpiperazin-1-yl)imidazo[1,2-b]pyridazin-2-yl)methyl)pyridin-4-amine (60 mg, 0.14 mmol), (1S,2S)-2-(3-chlorophenyl)cyclopropane-1-carboxamide (41 mg, 0.21 mmol), Pd₂(dba)₃ (13 mg, 0.014 mmol), Xantphos (8 mg, 0.014 mmol) and Cs₂CO₃ (137 mg, 0.42 mmol) in dioxane (5 mL) was stirred at 95° C. for 16 h under N₂. After cooling to room temperature, the reaction mixture was filtered through Celite® and the filter cake was washed with DCM/MeOH (10/1, 100 mL). The filtrate was concentrated in vacuo to give the crude, which was purified by column chromatography (eluent: DCM:MeOH=0-8%) to give (1S,2S)-2-(3-chlorophenyl)-N-(4-(((6-cyclopropyl-8-(4-methylpiperazin-1-yl)imidazo[1,2-b]pyridazin-2-yl)methyl)amino)pyridin-2-yl)cyclopropane-1-carboxamide as a light yellow solid (30 mg, 38%). ESI-MS [M+H]⁺: 557.3 ¹H NMR (400 MHz, DMSO) δ 10.34 (s, 1H), 7.76 (d, J=5.8 Hz, 1H), 7.70 (s, 1H), 7.46 (s, 1H), 7.33-7.29 (m, 1H), 7.26-7.24 (m, 2H), 7.15-7.13 (m, 1H), 7.10-7.07 (m, 1H), 6.35-6.33 (m, 1H), 6.16 (s, 1H), 4.32-4.31 (m, 2H), 3.93 (s, 4H), 2.51-2.50 (m, 4H), 2.40-2.39 (m, 2H), 2.24 (s, 3H), 1.98 (m, 1H), 1.47-1.43 (m, 1H), 1.38-1.33 (m, 1H), 0.96-0.85 (m, 4H).

### Example 41

### Synthesis of rac-(1S*,2S*)-6′-chloro-N-(4-(((6-cyclopropyl-[1,2,4]triazolo[1,5-a]pyrimidin-2-yl)methyl amino)pyridin-2-yl)-2′,3′-dihydrospiro[cyclopropane-1,1′-indene]-2-carboxamide (I-41)

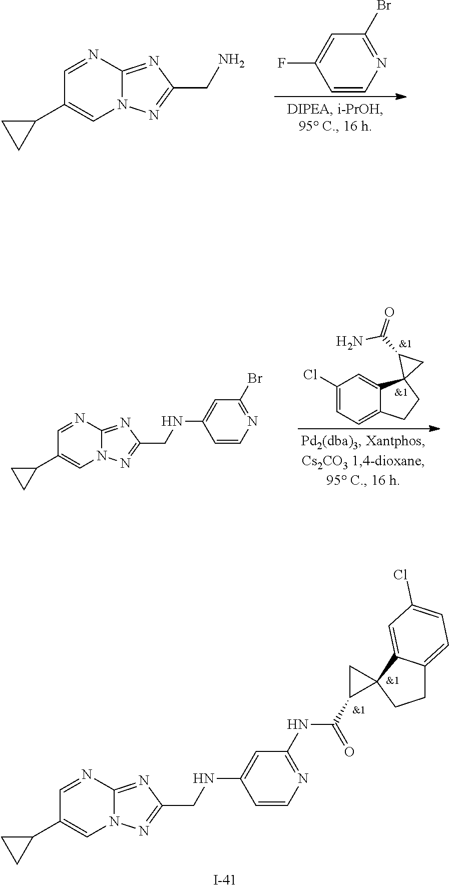 [SMILES: NCc1nc2ncc(C3CC3)cn2n1 ; Fc1ccnc(Br)c1 ; Brc1cc(NCc2nc3ncc(C4CC4)cn3n2)ccn1 ; NC(=O)[C@@H]1C[C@]12CCc1ccc(Cl)cc12 ; O=C(Nc1cc(NCc2nc3ncc(C4CC4)cn3n2)ccn1)[C@@H]1C[C@]12CCc1ccc(Cl)cc12 ; "I-41"]

Synthesis of 2-bromo-N-((6-cyclopropyl-[1,2,4]triazolo[1,5-a]pyrimidin-2-yl)methyl)pyridin-4-amine. A mixture of (6-cyclopropyl-[1,2,4]triazolo[1,5-a]pyrimidin-2-yl)methanamine (102 mg, 0.54 mmol), 2-bromo-4-fluoropyridine (142 mg, 0.81 mmol) and DIPEA (209 mg, 1.62 mmol) in i-PrOH (10 mL) was stirred at 95° C. for 16 h. The reaction mixture was concentrated in vacuo to give the crude, which was purified by preparative TLC (eluent: EtOAc) to give 2-bromo-N-((6-cyclopropyl-[1,2,4]triazolo[1,5-a]pyrimidin-2-yl)methyl)pyridin-4-amine (80 mg, 43%) as a white solid. ESI-MS [M+H]⁺: 345.2.

Synthesis of rac-(1S*,2S*)-6′-chloro-N-(4-(((6-cyclopropyl-[1,2,4]triazolo[1,5-a]pyrimidin-2-yl)methyl)amino)pyridin-2-yl)-2′,3′-dihydrospiro[cyclopropane-1,1′-indene]-2-carboxamide. To a mixture of 2-bromo-N-((6-cyclopropyl-[1,2,4]triazolo[1,5-a]pyrimidin-2-yl)methyl)pyridin-4-amine (30 mg, 0.087 mmol), rac-(1S*,2S*)-6′-chloro-2′,3′-dihydrospiro[cyclopropane-1,1′-indene]-2-carboxamide (29 mg, 0.13 mmol) and Cs₂CO₃ (85 mg, 0.26 mmol) in 1,4-dioxane (5 mL) was added Xantphos (10 mg, 0.017 mmol) and Pd₂(dba)₃ (8 mg, 0.009 mmol). The mixture was stirred at 95° C. for 16 h under N₂. The reaction mixture was cooled to room temperature then filtered through Celite® and the filter cake was washed with DCM/MeOH (10/1, 30 mL). The filtrate was concentrated in vacuo to give the crude, which was purified by preparative TLC (eluent: DCM/MeOH=20/1) to give rac-(1S*,2S*)-6′-chloro-N-(4-(((6-cyclopropyl-[1,2,4]triazolo[1,5-a]pyrimidin-2-yl)methyl)amino)pyridin-2-yl)-2′,3′-dihydrospiro[cyclopropane-1,1′-indene]-2-carboxamide (20 mg, 48%) as a white solid. ESI-MS [M+H]⁺: 486.2. ¹H NMR (400 MHz, DMSO) δ 10.21 (s, 1H), 9.14 (d, J=2.4 Hz, 1H), 8.74 (d, J=2.4 Hz, 1H), 7.74 (d, J=5.8 Hz, 1H), 7.44 (s, 1H), 7.28 (s, 1H), 7.23-7.16 (m, 2H), 6.88 (d, J=1.8 Hz, 1H), 6.33 (dd, J=5.8, 2.1 Hz, 1H), 4.52 (d, J=6.2 Hz, 2H), 3.00-2.77 (m, 2H), 2.44-2.41 (m, 1H), 2.20-2.01 (m, 3H), 1.58-1.41 (m, 2H), 1.08-0.96 (m, 2H), 0.93-0.77 (m, 2H).

### Example 42

### Synthesis of rac-(1S*,2S*)-2-(5-chloro-2-cyanophenyl)-N-(4-(((6-cyclopropyl-[1,2,4]triazolo[1,5-a]pyrimidin-2-yl)methyl amino)pyridin-2-yl)cyclopropane-1-carboxamide (I-42)

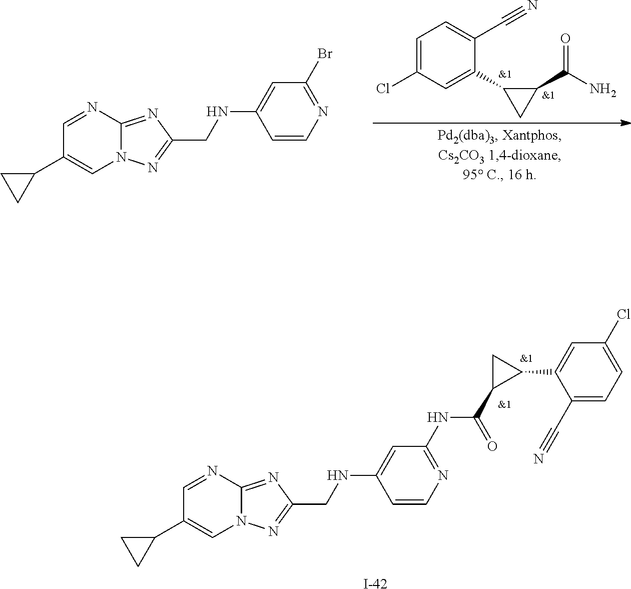 [SMILES: Brc1cc(NCc2nc3ncc(C4CC4)cn3n2)ccn1 ; N#Cc1ccc(Cl)cc1[C@H]1C[C@@H]1C(N)=O ; N#Cc1ccc(Cl)cc1[C@H]1C[C@@H]1C(=O)Nc1cc(NCc2nc3ncc(C4CC4)cn3n2)ccn1 ; "I-42"]

To a mixture of rac-(1S*,2S*)-2-(5-chloro-2-cyanophenyl)cyclopropane-carboxamide (31 mg, 0.14 mmol), 2-bromo-N-((6-cyclopropyl-[1,2,4]triazolo[1,5-a]pyrimidin-2-yl)methyl)pyridin-4-amine (48 mg, 0.14 mmol) and Cs₂CO₃ (137 mg, 0.42 mmol) in 1,4-dioxane (5 mL) was added Xantphos (16 mg, 0.028 mmol) and Pd₂(dba)₃ (13 mg, 0.014 mmol). The mixture was stirred at 95° C. for 16 h under N₂. The reaction mixture was cooled to room temperature. The mixture was filtered through Celite® and the filter cake was washed with DCM/MeOH (10/1, 30 mL). The filtrate was concentrated in vacuo to give the crude, which was purified by preparative TLC (eluent: DCM/MeOH=10/1) to give rac-(1S*,2S*)-2-(5-chloro-2-cyanophenyl)-N-(4-(((6-cyclopropyl-[1,2,4]triazolo[1,5-a]pyrimidin-2-yl)methyl)amino)pyridin-2-yl)cyclopropane-1-carboxamide (30 mg, 44%) as a white solid. ESI-MS [M+H]⁺: 485.2. ¹H NMR (400 MHz, DMSO) δ 10.39 (s, 1H), 9.14 (d, J=2.3 Hz, 1H), 8.74 (d, J=2.4 Hz, 1H), 7.86 (d, J=8.3 Hz, 1H), 7.78 (d, J=5.8 Hz, 1H), 7.51 (dd, J=8.3, 2.0 Hz, 1H), 7.43 (s, 1H), 7.38 (d, J=2.0 Hz, 1H), 7.34-7.27 (m, 1H), 6.36 (dd, J=5.7, 1.9 Hz, 1H), 4.53 (d, J=6.1 Hz, 2H), 2.58-2.54 (m, 1H), 2.49-2.43 (m, 1H), 2.13-2.01 (m, 1H), 1.62-1.48 (m, 2H), 1.07-0.96 (m, 2H), 0.93-0.81 (m, 2H).

### Example 43

### Synthesis of (1S,2S)-2-(3-chlorophenyl)-N-(6-(((6-cyclopropyl-8-(2-oxooxazolidin-3-yl)-[1,2,4]triazolo[1,5-a]pyridin-2-yl)methyl)amino)pyrimidin-4-yl)cyclopropane-1-carboxamide (I-43)

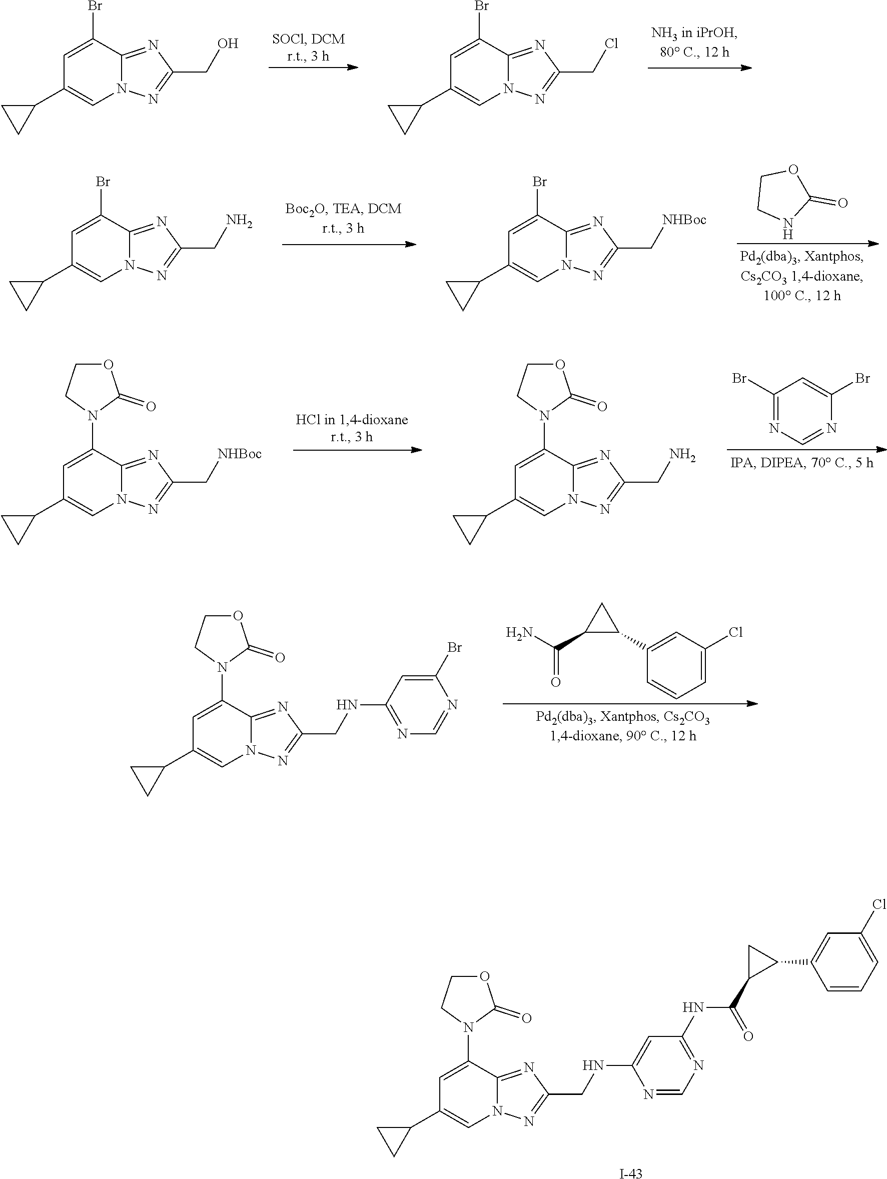

Synthesis of 8-bromo-2-(chloromethyl)-6-cyclopropyl-[1,2,4]triazolo[1,5-a]pyridine. A solution of (8-bromo-6-cyclopropyl-[1,2,4]triazolo[1,5-a]pyridin-2-yl)methanol (370 mg, 1.38 mmol)) in DCM (25 mL) and SOCl₂ (0.5 mL) was stirred at room temperature for 2 h. The mixture was concentrated in vacuo to give 8-bromo-2-(chloromethyl)-6-cyclopropyl-[1,2,4]triazolo[1,5-a]pyridine (400 mg, crude) as a yellow solid. ESI-MS [M+H]⁺: 286.1.

Synthesis of (8-bromo-6-cyclopropyl-[1,2,4]triazolo[1,5-a]pyridin-2-yl)methanamine. A solution of 8-bromo-2-(chloromethyl)-6-cyclopropyl-[1,2,4]triazolo[1,5-a]pyridine (400 mg, crude) in NH₃ solution (2N in iPrOH, 30 mL) was stirred in a sealed tube at 80° C. for 12 h. The mixture was cooled to room temperature and concentrated in vacuo to give (8-bromo-6-cyclopropyl-[1,2,4]triazolo[1,5-a]pyridin-2-yl)methanamine (450 mg, crude) as a white solid. ESI-MS [M+H]⁺: 267.1

Synthesis of tert-butyl ((8-bromo-6-cyclopropyl-[1,2,4]triazolo[1,5-a]pyridin-2-yl)methyl)carbamate. To a solution of (8-bromo-6-cyclopropyl-[1,2,4]triazolo[1,5-a]pyridin-2-yl)methanamine (450 mg crude) and TEA (570 mg, 5.64 mmol) in DCM (30 mL) was added Boc₂O (615 mg, 2.82 mmol) at 0° C. The resulting mixture was stirred at room temperature for 3 h under N₂ atmosphere. The reaction mixture was quenched with H₂O (30 mL) and then extracted with DCM (30 mL×3). The combined organics were washed with brine (50 mL), dried over Na₂SO₄, and concentrated in vacuo. The residue was purified by preparative TLC, eluting with 30% EtOAc in PE to give tert-butyl ((8-bromo-6-cyclopropyl-[1,2,4]triazolo[1,5-a]pyridin-2-yl)methyl)carbamate (290 mg, 57% over three steps) as a white solid. ESI-MS [M+H]⁺: 367.1

Synthesis of tert-butyl ((6-cyclopropyl-8-(2-oxooxazolidin-3-yl)-[1,2,4]triazolo[1,5-a]pyridin-2-yl)methyl)carbamate. A solution of tert-butyl ((8-bromo-6-cyclopropyl-[1,2,4]triazolo[1,5-a]pyridin-2-yl)methyl)carbamate (290 mg, 0.79 mmol), oxazolidin-2-one (137 mg, 1.58 mmol), Pd₂(dba)₃ (145 mg, 0.158 mmol), Xantphos (91 mg, 0.158 mmol) and Cs₂CO₃ (773 mg, 2.37 mmol) in 1,4-dioxane (30 mL) was stirred at 100° C. for 12 h under N₂. The mixture was cooled to room temperature and concentrated in vacuo. The residue was diluted with H₂O (30 mL) and extracted with EtOAc (50 mL×2). The combined organics were washed with brine (50 mL), dried over Na₂SO₄, and concentrated in vacuo. The residue was purified by preparative TLC, eluting with 50% EtOAc in PE to give tert-butyl ((6-cyclopropyl-8-(2-oxooxazolidin-3-yl)-[1,2,4]triazolo[1,5-a]pyridin-2-yl)methyl)carbamate (250 mg, 85%) as a yellow solid. ESI-MS [M+H]⁺: 374.2.

Synthesis of 3-(2-(aminomethyl)-6-cyclopropyl-[1,2,4]triazolo[1,5-a]pyridin-8-yl)oxazolidin-2-one. A solution of tert-butyl ((6-cyclopropyl-8-(2-oxooxazolidin-3-yl)-[1,2,4]triazolo[1,5-a]pyridin-2-yl)methyl)carbamate (250 mg, 0.67 mmol) in HCl (4.0 N in 1,4-dioxane, 30 mL) was stirred at room temperature for 3 h. The mixture was concentrated in vacuo to give 3-(2-(aminomethyl)-6-cyclopropyl-[1,2,4]triazolo[1,5-a]pyridin-8-yl)oxazolidin-2-one (250 mg, crude) as a yellow solid. ESI-MS [M+H]⁺: 274.2.

Synthesis of 3-(2-(((6-bromopyrimidin-4-yl)amino)methyl)-6-cyclopropyl-[1,2,4]triazolo[1,5-a]pyridin-8-yl)oxazolidin-2-one. A solution of 3-(2-(aminomethyl)-6-cyclopropyl-[1,2,4]triazolo[1,5-a]pyridin-8-yl)oxazolidin-2-one (250 mg, crude), 4,6-dibromopyrimidine (268 mg, 1.125 mmol), and DIPEA (290 mg, 2.25 mmol) in iPrOH (25 mL) was stirred at 70° C. for 5 under N₂ atmosphere. The mixture was cooled to room temperature and concentrated in vacuo. The residue was purified by preparative TLC, eluting with 10% MeOH in DCM to give 3-(2-(((6-bromopyrimidin-4-yl)amino)methyl)-6-cyclopropyl-[1,2,4]triazolo[1,5-a]pyridin-8-yl)oxazolidin-2-one (225 mg, 78%, two steps) as a white solid. ESI-MS [M+H]⁺: 430.1.

Synthesis of (1S,2S)-2-(3-chlorophenyl)-N-(6-(((6-cyclopropyl-8-(2-oxooxazolidin-3-yl)-[1,2,4]triazolo[1,5-a]pyridin-2-yl)methyl)amino)pyrimidin-4-yl)cyclopropane-1-carboxamide. A solution of 3-(2-(((6-bromopyrimidin-4-yl)amino)methyl)-6-cyclopropyl-[1,2,4]triazolo[1,5-a]pyridin-8-yl)oxazolidin-2-one (100 mg, 0.233 mmol), (1S,2S)-2-(3-chlorophenyl)cyclopropane-1-carboxamide (55 mg, 0.280 mmol), Pd₂(dba)₃ (43 mg, 0.0466 mmol), Xantphos (27 mg, 0.0466 mmol), and Cs₂CO₃ (228 mg, 0.699 mmol) in 1,4-dioxane (20 mL) was stirred at 90° C. for 12 h under N₂. The mixture was cooled to room temperature and concentrated in vacuo. The residue was diluted with H₂O (30 mL) and extracted with EtOAc (30 mL×3). The combined organics were washed with brine (50 mL), dried over Na₂SO₄, and concentrated in vacuo. The residue was purified by preparative TLC, eluting with 10% MeOH in DCM to give (1S,2S)-2-(3-chlorophenyl)-N-(6-(((6-cyclopropyl-8-(2-oxooxazolidin-3-yl)-[1,2,4]triazolo[1,5-a]pyridin-2-yl)methyl)amino)pyrimidin-4-yl)cyclopropane-1-carboxamide as a white solid. (35 mg, 27%). ESI-MS [M+H]⁺: 545.2. ¹H NMR (400 MHz, DMSO) δ 10.62 (s, 1H), 8.62 (d, J=1.2 Hz, 1H), 8.17 (s, 1H), 8.02 (s, 1H), 7.67 (d, J=1.3 Hz, 1H), 7.35-7.24 (m, 4H), 7.15 (d, J=7.6 Hz, 1H), 4.72 (s, 2H), 4.59-4.37 (m, 4H), 2.46-2.29 (m, 2H), 2.12-1.93 (m, 1H), 1.54-1.35 (m, 2H), 1.05-0.88 (m, 2H), 0.82-0.67 (m, 2H).

### Example 44

### Synthesis of (1S,2S)-2-(3-chlorophenyl)-N-(6-(((6-cyclopropyl-8-(2-oxooxazolidin-3-yl)imidazo[1,2-b]pyridazin-2-yl)methyl)amino)pyrimidin-4-yl)cyclopropane-1-carboxamide (I-44)

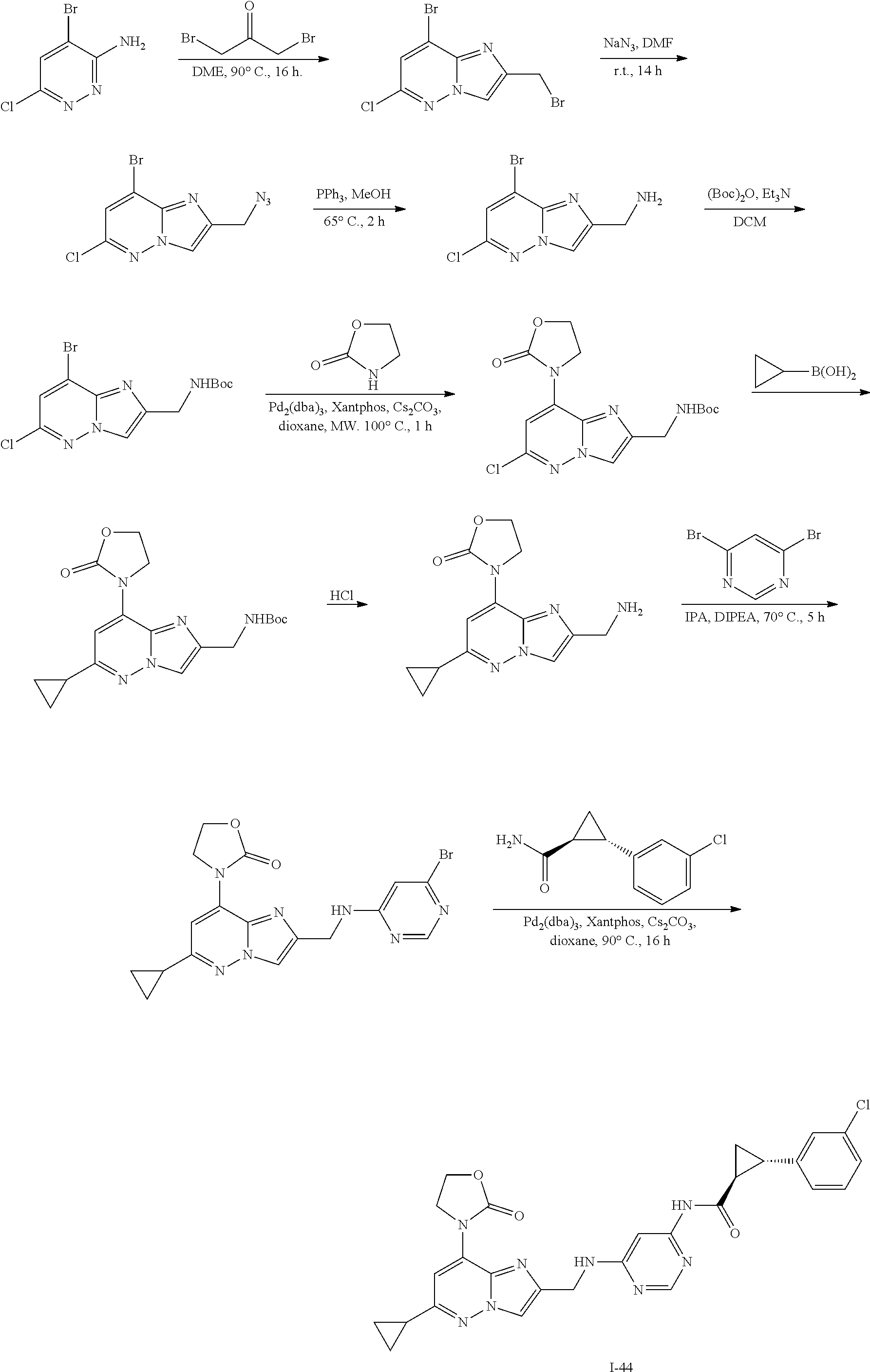

Synthesis of 8-bromo-2-(bromomethyl)-6-chloroimidazo[1,2-b]pyridazine. A mixture of 4-bromo-6-chloropyridazin-3-amine (5.0 g, 24 mmol) and 1,3-dibromopropan-2-one (15.6 g, 72 mmol) in DME (100 mL) was stirred at 90° C. for 16 h under N₂. After the reaction was cooled to room temperature, NaHCO₃ (sat. aq., 150 mL) was added and the reaction was extracted with EtOAc (100 mL×3). The combined organic layers were washed with brine (50 mL), dried over Na₂SO₄ and concentrated in vacuo to give the crude, which was purified by column chromatography (eluent: DCM/MeOH=50/1) to give 8-bromo-2-(bromomethyl)-6-chloroimidazo[1,2-b]pyridazine (6.5 g, 83%) as a yellow solid. ESI-MS [M+H]⁺: 325.9.

Synthesis of 2-(azidomethyl)-8-bromo-6-chloroimidazo[1,2-b]pyridazine. A mixture of 8-bromo-2-(bromomethyl)-6-chloroimidazo[1,2-b]pyridazine (1.2 g, 3.69 mmol) and NaN₃ (240 mg, 3.69 mmol) in DMF (15 mL) was stirred at room temperature for 3 h under N₂. The reaction was quenched with water (150 mL) and extracted with DCM (50 mL×3). The combined organic layers were washed with brine (50 mL), dried over Na₂SO₄ and concentrated in vacuo to give 2-(azidomethyl)-8-bromo-6-chloroimidazo[1,2-b]pyridazine (1.1 g, crude) as a yellow solid. ESI-MS [M+H]⁺: 287.1.

Synthesis of (8-bromo-6-chloroimidazo[1,2-b]pyridazin-2-yl)methanamine. A mixture of 2-(azidomethyl)-8-bromo-6-chloroimidazo[1,2-b]pyridazine (1.1 g, crude) and PPh₃ (1.1 g, 4.25 mmol) in MeOH (25 mL) was stirred at 60° C. for 2 h under N₂. After cooling to room temperature, the mixture was concentrated in vacuo and purified by column chromatography (eluent: DCM/MeOH=10/1) to give (8-bromo-6-chloroimidazo[1,2-b]pyridazin-2-yl)methanamine as a light yellow oil (500 mg, 52%). ESI-MS [M+H]⁺: 261.2.

Synthesis of tert-butyl ((8-bromo-6-chloroimidazo[1,2-b]pyridazin-2-yl)methyl)carbamate. A mixture of (8-bromo-6-chloroimidazo[1,2-b]pyridazin-2-yl)methanamine (520 mg, 2.0 mmol), (Boc)₂O (654 mg, 3.0 mmol) and Et₃N (0.83 mL, 6.0 mmol) in DCM (20.0 mL) was stirred at room temperature under N₂ for 16 h. The reaction was quenched with NaHCO₃ (sat. aq., 30 mL) and extracted with DCM (30 mL×3). The combined organic layers were washed with brine (30 mL), dried over Na₂SO₄ then concentrated in vacuo to give the crude, which was purified by preparative TLC (eluent: MeOH/DCM=1/20) to give tert-butyl ((8-bromo-6-chloroimidazo[1,2-b]pyridazin-2-yl)methyl)carbamate (200 mg, 28%) as a yellow solid. ESI-MS [M+H]⁺: 361.2

Synthesis of tert-butyl ((6-chloro-8-(2-oxooxazolidin-3-yl)imidazo[1,2-b]pyridazin-2-yl)methyl)carbamate. A mixture of e tert-butyl ((8-bromo-6-chloroimidazo[1,2-b]pyridazin-2-yl)methyl)carbamate (150 mg, 0.42 mmol), oxazolidin-2-one (32 mg, 0.37 mmol), Pd₂(dba)₃ (19 mg, 0.021 mmol), Xantphos (12 mg, 0.021 mmol) and Cs₂CO₃ (342 mg, 1.05 mmol) in dioxane (8.0 mL) was stirred in a sealed tube. After degassing with N₂ for 1 min, the reaction was irradiated in microwave at 100° C. for 1 h. After cooling to room temperature, the reaction mixture was filtered through Celite® and the filter cake was washed with DCM/MeOH (10/1, 50 mL). The filtrate was concentrated in vacuo to give the crude, which was purified by preparative TLC (eluent: DCM/MeOH=15/1) to give the product tert-butyl ((6-chloro-8-(2-oxooxazolidin-3-yl)imidazo[1,2-b]pyridazin-2-yl)methyl)carbamate (90 mg, 66%) as a yellow solid. ESI-MS [M+H]⁺: 368.2.

Synthesis of tert-butyl ((6-cyclopropyl-8-(2-oxooxazolidin-3-yl)imidazo[1,2-b]pyridazin-2-yl)methyl)carbamate. A mixture of tert-butyl ((6-chloro-8-(2-oxooxazolidin-3-yl)imidazo[1,2-b]pyridazin-2-yl)methyl)carbamate (100.0 mg, 0.27 mmol), cyclopropylboronic acid (70 mg, 0.81 mmol), Pd(OAc)₂ (6.0 mg, 0.027 mmol), S-phos (11 mg, 0.027 mmol) and K₃PO₄ (172 mg, 0.81 mmol) in Tol/water (10.0 mL/1.0 mL) was stirred at 95° C. under N₂ for 16 h. After cooling to room temperature, the reaction was quenched with water (30 mL) and extracted with EtOAc (20 mL×3). The combined organic layers were washed with brine (30 mL), dried over Na₂SO₄ then concentrated in vacuo to give the crude, which was purified by preparative TLC (eluent: MeOH/DCM=1/15) to give the desired product tert-butyl ((6-cyclopropyl-8-(2-oxooxazolidin-3-yl)imidazo[1,2-b]pyridazin-2-yl)methyl)carbamate (80 mg, 80%) as a yellow solid. ESI-MS [M+H]⁺: 374.1.

Synthesis of 3-(2-(aminomethyl)-6-cyclopropylimidazo[1,2-b]pyridazin-8-yl)oxazolidin-2-one. A mixture of tert-butyl ((6-cyclopropyl-8-(2-oxooxazolidin-3-yl)imidazo[1,2-b]pyridazin-2-yl)methyl)carbamate (80 mg, 0.21 mmol) in HCl (4M in dioxane, 2.0 mL) was stirred at room temperature for 1 h. The mixture was concentrated in vacuo to give 3-(2-(aminomethyl)-6-cyclopropylimidazo[1,2-b]pyridazin-8-yl)oxazolidin-2-one (60 mg, crude) as a yellow solid which was used for the next step directly. ESI-MS [M+H]⁺: 274.2.

Synthesis of 3-(2-(((6-bromopyrimidin-4-yl)amino)methyl)-6-cyclopropylimidazo[1,2-b]pyridazin-8-yl)oxazolidin-2-one. A mixture of 3-(2-(aminomethyl)-6-cyclopropylimidazo[1,2-b]pyridazin-8-yl)oxazolidin-2-one (60 mg, 0.21 mmol), 4,6-dibromopyrimidine (76 mg, 0.32 mmol) and DIPEA (142 mg, 1.1 mmol) in i-PrOH (5 mL) was stirred at 70° C. under N₂ for 4 h. After cooling to room temperature, the reaction was quenched with NaHCO₃ (sat. aq., 30 mL) and extracted with DCM (30 mL×3). The combined organic layers were washed with brine (30 mL), dried over Na₂SO₄ then concentrated in vacuo to give the crude, which was purified by preparative TLC (eluent: MeOH/DCM=1/20) to give 3-(2-(((6-bromopyrimidin-4-yl)amino)methyl)-6-cyclopropylimidazo[1,2-b]pyridazin-8-yl)oxazolidin-2-one (70 mg, 78%, for two steps) as a yellow solid. ESI-MS [M+H]⁺: 430.2.

Synthesis of (1S,2S)-2-(3-chlorophenyl)-N-(6-(((6-cyclopropyl-8-(2-oxooxazolidin-3-yl)imidazo[1,2-b]pyridazin-2-yl)methyl)amino)pyrimidin-4-yl)cyclopropane-1-carboxamide. A mixture of 3-(2-(((6-bromopyrimidin-4-yl)amino)methyl)-6-cyclopropylimidazo[1,2-b]pyridazin-8-yl)oxazolidin-2-one (70 mg, 0.16 mmol), (1S,2S)-2-(3-chlorophenyl)cyclopropane-1-carboxamide (31 mg, 0.16 mmol), Pd₂(dba)₃ (15 mg, 0.016 mmol), Xantphos (9 mg, 0.016 mmol) and Cs₂CO₃ (130 mg, 0.40 mmol) in 1,4-dioxane (6.0 mL) was stirred at 90° C. under N₂ for 16 h. After cooling to room temperature, the reaction mixture was filtered through Celite® and the filter cake was washed with DCM/MeOH (10/1, 50 mL). The filtrate was concentrated in vacuo to give the crude, which was purified by preparative TLC (DCM/MeOH=15/1) to give (1S,2S)-2-(3-chlorophenyl)-N-(6-(((6-cyclopropyl-8-(2-oxooxazolidin-3-yl)imidazo[1,2-b]pyridazin-2-yl)methyl)amino)pyrimidin-4-yl)cyclopropane-1-carboxamide (22 mg, 25%) as a white solid. ESI-MS [M+H]⁺: 545.1. ¹H NMR (400 MHz, DMSO) δ 10.62 (s, 1H), 8.20 (s, 1H), 7.90 (s, 1H), 7.85 (s, 1H), 7.57 (s, 1H), 7.39-7.21 (m, 4H), 7.15 (d, J=7.6 Hz, 1H), 4.79 (t, J=8.0 Hz, 2H), 4.58 (s, 2H), 4.51 (t, J=8.0 Hz, 2H), 2.45-2.29 (m, 2H), 2.21-2.04 (m, 1H), 1.58-1.33 (m, 2H), 1.10-0.96 (m, 2H), 0.97-0.80 (m, 2H).

### Example 45

### Synthesis of (1S,2S)-2-(3-chlorophenyl)-N-(4-(((6-cyclopropyl-8-(4-methylpiperazin-1-yl)-[1,2,4]triazolo[1,5-a]pyridin-2-yl)methyl)amino)pyridin-2-yl)cyclopropane-1-carboxamide (I-45)

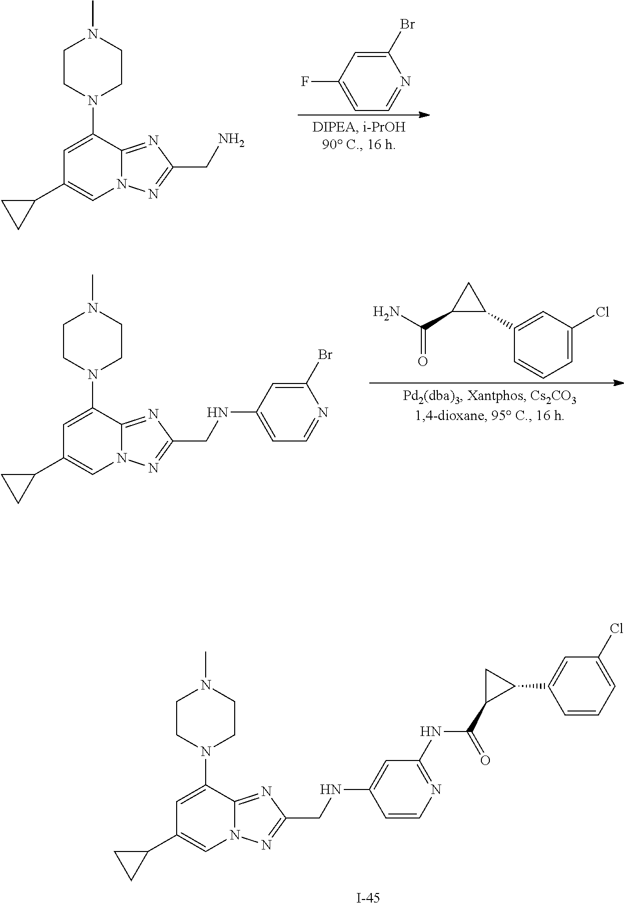 [SMILES: CN1CCN(c2cc(C3CC3)cn3nc(CN)nc23)CC1 ; Fc1ccnc(Br)c1 ; CN1CCN(c2cc(C3CC3)cn3nc(CNc4ccnc(Br)c4)nc23)CC1 ; NC(=O)[C@H]1C[C@@H]1c1cccc(Cl)c1 ; CN1CCN(c2cc(C3CC3)cn3nc(CNc4ccnc(NC(=O)[C@H]5C[C@@H]5c5cccc(Cl)c5)c4)nc23)CC1 ; "I-45"]

Synthesis of 2-bromo-N-((6-cyclopropyl-8-(4-methylpiperazin-1-yl)-[1,2,4]triazolo[1,5-a]pyridin-2-yl)methyl)pyridin-4-amine. A mixture of (6-cyclopropyl-8-(4-methylpiperazin-1-yl)-[1,2,4]triazolo[1,5-a]pyridin-2-yl)methanamine (45 mg, 0.16 mmol), 2-bromo-4-fluoropyridine (42 mg, 0.24 mmol) and DIPEA (103 mg, 0.8 mmol) in i-PrOH (10 mL) was stirred at 90° C. for 16 h. The reaction mixture was concentrated in vacuo to give the crude, which was purified by preparative TLC (eluent: DCM/MeOH=10/1) to give 2-bromo-N-((6-cyclopropyl-8-(4-methylpiperazin-1-yl)-[1,2,4]triazolo[1,5-a]pyridin-2-yl)methyl)pyridin-4-amine (15 mg, 21%) as a white solid. ESI-MS [M+H]⁺: 444.2.

Synthesis of (1S,2S)-2-(3-chlorophenyl)-N-(4-(((6-cyclopropyl-8-(4-methylpiperazin-1-yl)-[1,2,4]triazolo[1,5-a]pyridin-2-yl)methyl)amino)pyridin-2-yl)cyclopropane-1-carboxamide. To a mixture of 2-bromo-N-((6-cyclopropyl-8-(4-methylpiperazin-1-yl)-[1,2,4]triazolo[1,5-a]pyridin-2-yl)methyl)pyridin-4-amine (15 mg, 0.034 mmol), (1S,2S)-2-(3-chlorophenyl)cyclopropane-1-carboxamide (10 mg, 0.05 mmol) and Cs₂CO₃ (33 mg, 0.10 mmol) in 1,4-dioxane (5 mL) was added Xantphos (7 mg, 0.012 mmol) and Pd₂(dba)₃ (5 mg, 0.006 mmol). The mixture was stirred at 95° C. for 16 h under N₂. The reaction mixture was cooled to room temperature. The mixture was filtered through Celite® and the filter cake was washed with DCM/MeOH (10/1, 20 mL). The filtrate was concentrated in vacuo to give the crude, which was purified by preparative TLC (eluent: DCM/MeOH=10/1) to give (1S,2S)-2-(3-chlorophenyl)-N-(4-(((6-cyclopropyl-8-(4-methylpiperazin-1-yl)-[1,2,4]triazolo[1,5-a]pyridin-2-yl)methyl)amino)pyridin-2-yl)cyclopropane-1-carboxamide (8 mg, 42%) as a white solid. ESI-MS [M+H]⁺: 557.2. ¹H NMR (400 MHz, DMSO) δ 10.30 (s, 1H), 8.19 (s, 1H), 7.76 (d, J=5.8 Hz, 1H), 7.45 (s, 1H), 7.35-7.18 (m, 4H), 7.14 (d, J=7.7 Hz, 1H), 6.50 (d, J=1.1 Hz, 1H), 6.34 (dd, J=5.8, 2.2 Hz, 1H), 4.45 (d, J=5.9 Hz, 2H), 3.50-3.41 (m, 4H), 2.50-2.49 (m, 4H), 2.43-2.30 (m, 2H), 2.23 (s, 3H), 1.99-1.92 (m, 1H), 1.51-1.40 (m, 1H), 1.37-1.32 (m, 1H), 0.96-0.87 (m, 2H), 0.79-0.75 (m, 2H).

### Example 46

### Synthesis of (1S,2S)-2-(3-chlorophenyl)-N-(4-(((6-cyclopropylimidazo[1,2-b]pyridazin-2-yl)methyl)amino)pyridin-2-yl)cyclopropane-1-carboxamide (I-46)

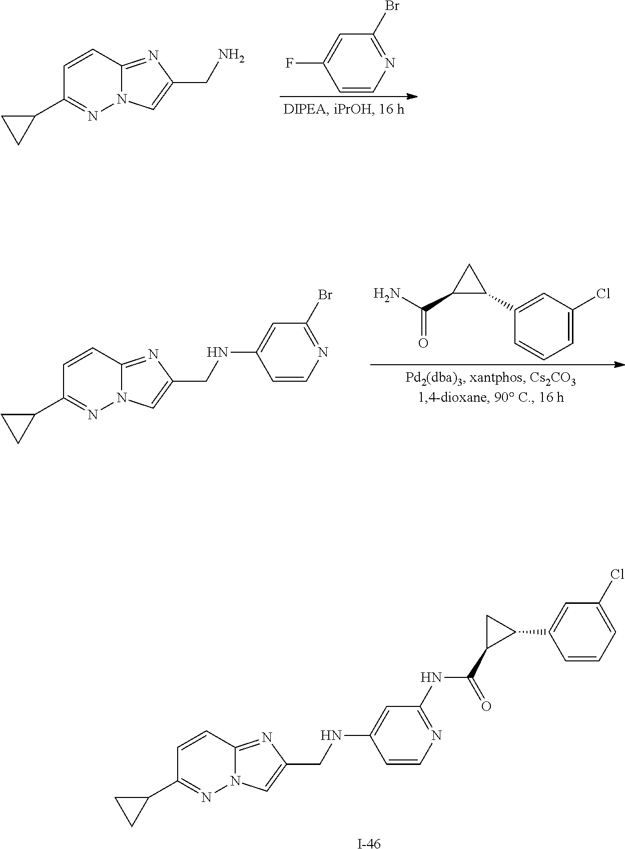 [SMILES: NCc1cn2nc(C3CC3)ccc2n1 ; Fc1ccnc(Br)c1 ; Brc1cc(NCc2cn3nc(C4CC4)ccc3n2)ccn1 ; NC(=O)[C@H]1C[C@@H]1c1cccc(Cl)c1 ; O=C(Nc1cc(NCc2cn3nc(C4CC4)ccc3n2)ccn1)[C@H]1C[C@@H]1c1cccc(Cl)c1 ; "I-46"]

Synthesis of 2-bromo-N-((6-cyclopropylimidazo[1,2-b]pyridazin-2-yl)methyl)pyridin-4-amine. A mixture of (6-cyclopropylimidazo[1,2-b]pyridazin-2-yl)methanamine (230 mg, 1.22 mmol), DIPEA (629.5 mg, 4.88 mmol) and 2-bromo-4-fluoropyridine (429 mg, 2.44 mmol) in iPrOH (20 mg) was stirred at 110° C. for 16 h under N₂. The reaction was concentrated in vacuo to give the crude product, which was purified by silica gel column chromatography eluting with a gradient of 1-8% MeOH in DCM to give 2-bromo-N-((6-cyclopropylimidazo[1,2-b]pyridazin-2-yl)methyl)pyridin-4-amine (160 mg, 38%) as a yellow oil. ESI-MS [M+H]⁺: 344.0.

Synthesis of (1S,2S)-2-(3-chlorophenyl)-N-(4-(((6-cyclopropylimidazo[1,2-b]pyridazin-2-yl)methyl)amino)pyridin-2-yl)cyclopropane-1-carboxamide. To a mixture of 2-bromo-N-((6-cyclopropylimidazo[1,2-b]pyridazin-2-yl)methyl)pyridin-4-amine (40.0 mg, 0.12 mmol) in 1,4-dioxane (5 mL) was added (1S,2S)-2-(3-chlorophenyl)cyclopropane-1-carboxamide (35.1 mg, 0.18 mmol), Pd₂(dba)₃ (21.1 mg, 0.023 mmol), Xantphos (13.3 mg, 0.023 mmol), Cs₂CO₃ (117.4 mg, 0.36 mmol). The reaction mixture was stirred at 90° C. for 16 h under N₂. The reaction mixture was cooling to room temperature, filtered through Celite® and the filter cake was washed with DCM/MeOH (10/1, 40 mL). The filtrate was concentrated in vacuo to give the crude, which was purified by preparative TLC (eluent: DCM/MeOH=10/1) to give (1S,2S)-2-(3-chlorophenyl)-N-(4-(((6-cyclopropylimidazo[1,2-b]pyridazin-2-yl)methyl)amino)pyridin-2-yl)cyclopropane-1-carboxamide (8 mg, 15.0%) as a white solid. ESI-MS [M+H]⁺: 459.2. ¹H NMR (400 MHz, DMSO) δ 10.33 (s, 1H), 7.94-7.90 (m, 2H), 7.77-7.75 (d, J=5.8 Hz, 1H), 7.42 (s, 1H), 7.33-7.29 (m, 1H), 7.26-7.24 (m, 2H), 7.15-7.05 (m, 3H), 6.35-6.33 (m, 1H), 4.38-4.37 (d, J=5.5 Hz, 2H), 2.40-2.32 (m, 2H), 2.20-2.13 (m, 1H), 1.47-1.42 (m, 1H), 1.37-1.32 (m, 1H), 1.08-1.03 (m, 2H), 0.98-0.94 (m, 2H).

### Example 47

### Synthesis of ethyl 3-(2-(((6-((S,2S)-2-(3-chlorophenyl)cyclopropane-1-carboxamido)pyrimidin-4-yl)amino)methyl)-5-cyclopropylpyrazolo[1,5-a]pyridin-7-yl)propanoate (I-47)

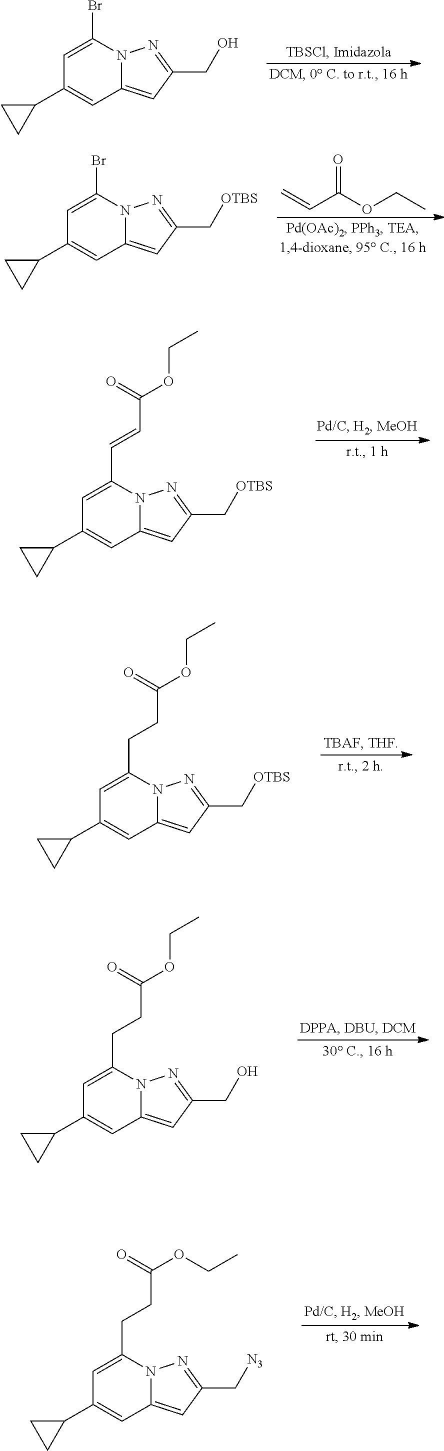

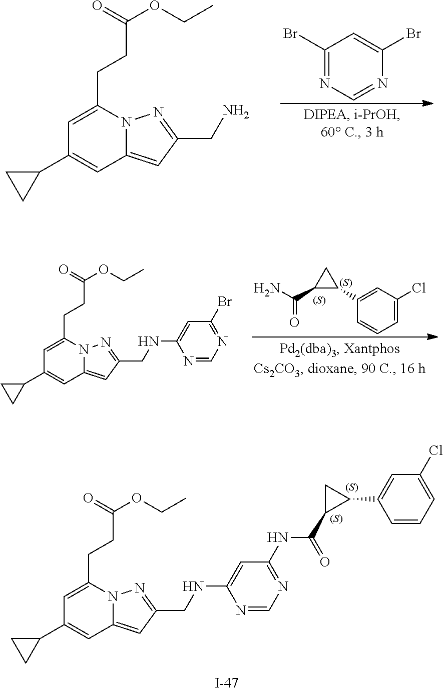

Synthesis of 7-bromo-2-(((tert-butyldimethylsilyl)oxy)methyl)-5-cyclopropylpyrazolo[1,5-a]pyridine. To a solution of (7-bromo-5-cyclopropylpyrazolo[1,5-a]pyridin-2-yl)methanol (1.5 g, 5.6 mmol) and imidazole (1.1 g, 16.8 mmol) in DCM (30 mL) was added TBSCl (1.26 g, 8.4 mmol) at room temperature. The reaction mixture was stirred at room temperature for 16 h. H₂O (100 mL) was added and the mixture was extracted with DCM (50 mL×3). The combined organic layers were washed with brine (50 mL), dried over anhydrous Na₂SO₄, and concentrated in vacuo to give the crude, which was purified by column chromatography (eluent: PE/EtOAc=5/1) to give 7-bromo-2-(((tert-butyldimethylsilyl)oxy)methyl)-5-cyclopropylpyrazolo[1,5-a]pyridine (1.5 g, 70%) as a yellow oil. ESI-MS [M+H]⁺: 383.2.

Synthesis of ethyl (E)-3-(2-(((tert-butyldimethylsilyl)oxy)methyl)-5-cyclopropylpyrazolo[1,5-a]pyridin-7-yl)acrylate. A mixture of 7-bromo-2-(((tert-butyldimethylsilyl)oxy)methyl)-5-cyclopropylpyrazolo[1,5-a]pyridine (1.5 g, 3.9 mmol), ethyl acrylate (3.9 g, 39.0 mmol), Pd(OAc)₂ (87 mg, 0.39 mmol), PPh₃ (102 mg, 0.39 mmol) and TEA (3.9 g, 39.0 mmol) in 1,4-dioxane (100 mL) was stirred at 95° C. for 16 h under N₂. The reaction mixture was cooled to room temperature. The mixture was filtered through Celite® and the filter cake was washed with DCM/MeOH (10/1, 100 mL). The filtrate was concentrated in vacuo to give the crude, which was purified by column chromatography (eluent: PE/EtOAc=5/1) to give ethyl (E)-3-(2-(((tert-butyldimethylsilyl)oxy)methyl)-5-cyclopropylpyrazolo[1,5-a]pyridin-7-yl)acrylate (1.2 g, 77%) as a yellow oil. ESI-MS [M+H]⁺: 401.2.

Synthesis of ethyl 3-(2-(((tert-butyldimethylsilyl)oxy)methyl)-5-cyclopropylpyrazolo[1,5-a]pyridin-7-yl)propanoate. A mixture of ethyl (E)-3-(2-(((tert-butyldimethylsilyl)oxy)methyl)-5-cyclopropylpyrazolo[1,5-a]pyridin-7-yl)acrylate (1.2 g, 3.0 mmol) and Pd/C (100 mg) in MeOH (30 mL) was stirred at room temperature for 1 h under H₂. The mixture was filtered through Celite® and the filter cake was washed with DCM/MeOH (10/1, 50 mL). The filtrate was concentrated in vacuo to give the crude, which was purified by column chromatography (eluent: PE/EtOAc=2/1) to give ethyl 3-(2-(((tert-butyldimethylsilyl)oxy)methyl)-5-cyclopropylpyrazolo[1,5-a]pyridin-7-yl)propanoate (1.0 g, 83%) as a yellow oil. ESI-MS [M+H]⁺: 403.2.

Synthesis of ethyl 3-(5-cyclopropyl-2-(hydroxymethyl)pyrazolo[1,5-a]pyridin-7-yl)propanoate. To a solution of ethyl 3-(2-(((tert-butyldimethylsilyl)oxy)methyl)-5-cyclopropylpyrazolo[1,5-a]pyridin-7-yl)propanoate (1.0 g, 2.5 mmol) in THF (20 mL) was added TBAF (1M solution in THF, 5.0 mL, 5.0 mmol). The reaction mixture was stirred at room temperature for 2 h then diluted with H₂O (30 mL), and extracted with DCM (30 mL×3). The combined organic layers were washed with brine (40 mL), dried over anhydrous Na₂SO₄, and concentrated to give the crude, which was purified by column chromatography (eluent: DCM/MeOH=20/1) to give ethyl 3-(5-cyclopropyl-2-(hydroxymethyl)pyrazolo[1,5-a]pyridin-7-yl)propanoate (500 mg, 69%) as a yellow oil. ESI-MS [M+H]⁺: 289.2.

Synthesis of ethyl 3-(2-(azidomethyl)-5-cyclopropylpyrazolo[1,5-a]pyridin-7-yl)propanoate. To a mixture of ethyl 3-(5-cyclopropyl-2-(hydroxymethyl)pyrazolo[1,5-a]pyridin-7-yl)propanoate (100 mg, 0.35 mmol) in DCM (5 mL) was added DPPA (289 mg, 1.05 mmol) at 0° C., then a solution of DBU (160 mg, 1.05 mmol) in DCM (1 mL) was added. The reaction mixture was stirred at 35° C. under N₂ for 16 h. The reaction mixture was cooled to room temperature and diluted with H₂O (30 mL), extracted with DCM (30 mL×3). The combined organic layers were washed with brine (40 mL), dried over anhydrous Na₂SO₄, and concentrated in vacuo to give the crude product, which was purified by preparative TLC (eluent: PE/EtOAc=10/1) to give ethyl 3-(2-(azidomethyl)-5-cyclopropylpyrazolo[1,5-a]pyridin-7-yl)propanoate (80 mg, 73%) as a yellow solid. ESI-MS [M+H]⁺: 314.2.

Synthesis of ethyl 3-(2-(aminomethyl)-5-cyclopropylpyrazolo[1,5-a]pyridin-7-yl)propanoate. To a mixture of ethyl 3-(2-(azidomethyl)-5-cyclopropylpyrazolo[1,5-a]pyridin-7-yl)propanoate (80 mg, 0.26 mmol) in MeOH (5 mL) was added Pd/C (10 mg). The reaction mixture was stirred at room temperature for 30 min under H₂. The reaction mixture was filtered through Celite® and the filter cake was washed with DCM/MeOH (10/1, 20 mL). The filtrate was concentrated in vacuo to give ethyl 3-(2-(aminomethyl)-5-cyclopropylpyrazolo[1,5-a]pyridin-7-yl)propanoate (70 mg, 94%) as a yellow oil. ESI-MS [M+H]⁺: 288.2.

Synthesis of ethyl 3-(2-(((6-bromopyrimidin-4-yl)amino)methyl)-5-cyclopropylpyrazolo[1,5-a]pyridin-7-yl)propanoate. A mixture of ethyl 3-(2-(aminomethyl)-5-cyclopropylpyrazolo[1,5-a]pyridin-7-yl)propanoate (70 mg, 0.24 mmol), 4,6-dibromopyrimidine (86 mg, 0.36 mmol) and DIPEA (155 mg, 1.2 mmol) in i-PrOH (5 mL) was stirred at 60° C. for 3 h. The mixture was concentrated in vacuo to give the crude product, which was purified by preparative TLC (eluent: PE/EtOAc=2/1) to give ethyl 3-(2-(((6-bromopyrimidin-4-yl)amino)methyl)-5-cyclopropylpyrazolo[1,5-a]pyridin-7-yl)propanoate (70 mg, 66%) as a yellow oil. ESI-MS [M+H]⁺: 444.0.

Synthesis of ethyl 3-(2-(((6-((S,2S)-2-(3-chlorophenyl)cyclopropane-1-carboxamido)pyrimidin-4-yl)amino)methyl)-5-cyclopropylpyrazolo[1,5-a]pyridin-7-yl)propanoate. To a mixture of ethyl 3-(2-(((6-bromopyrimidin-4-yl)amino)methyl)-5-cyclopropylpyrazolo[1,5-a]pyridin-7-yl)propanoate (75 mg, 0.17 mmol), (1S,2S)-2-(3-chlorophenyl)cyclopropane-1-carboxamide (33 mg, 0.17 mmol) and Cs₂CO₃ (166 mg, 0.51 mmol) in 1,4-dioxane (5 mL) was added Pd₂(dba)₃ (16 mg, 0.017 mmol) and Xantphos (20 mg, 0.034 mmol). The mixture was stirred at 90° C. for 16 h under N₂. The reaction mixture was cooled to room temperature. The mixture was filtered through Celite® and the filter cake was washed with DCM/MeOH (10/1, 30 mL). The filtrate was concentrated in vacuo to give the crude, which was purified by preparative TLC (eluent: DCM/MeOH=15/1) to give ethyl 3-(2-(((6-((1S,2S)-2-(3-chlorophenyl)cyclopropane-1-carboxamido)pyrimidin-4-yl)amino)methyl)-5-cyclopropylpyrazolo[1,5-a]pyridin-7-yl)propanoate (70 mg, 74%) as a yellow solid. ESI-MS [M+H]⁺: 559.2. ¹H NMR (400 MHz, DMSO) δ 10.60 (s, 1H), 8.20 (s, 1H), 7.90 (s, 1H), 7.33-7.20 (m, 5H), 7.15 (d, J=7.6 Hz, 1H), 6.42 (s, 1H), 6.31 (s, 1H), 4.64 (s, 2H), 4.08 (q, J=7.1 Hz, 2H), 3.27-3.25 (m, 2H), 2.88-2.85 (m, 2H), 2.43-2.34 (m, 2H), 1.96-1.90 (m, 1H), 1.45-4.12 (m, 1H), 1.43-1.38 (m, 1H), 1.17 (t, J=7.1 Hz, 3H), 1.00-0.94 (m, 2H), 0.75-0.71 (m, 2H).

### Example 48

### Synthesis of 3-(2-(((6-((1S,2S)-2-(3-chlorophenyl)cyclopropane-1-carboxamido)pyrimidin-4-yl)amino)methyl)-5-cyclopropylpyrazolo[1,5-a]pyridin-7-yl)propanoic acid (1-48)

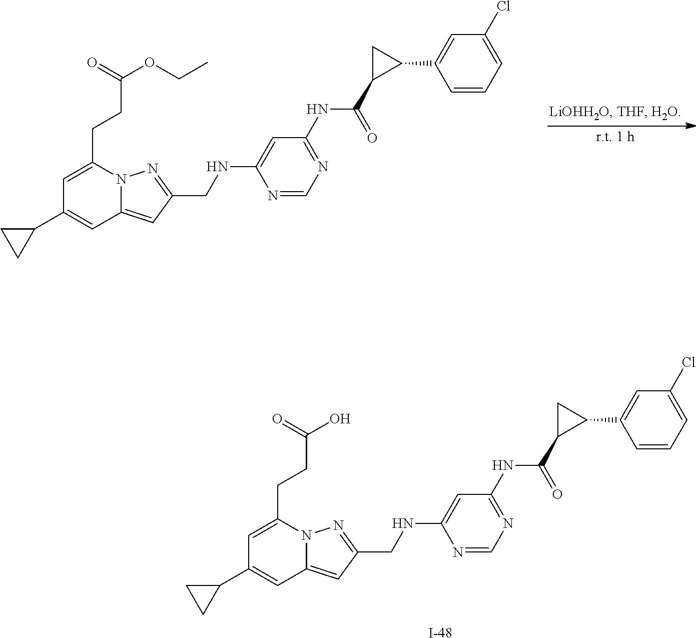

To a mixture of ethyl 3-(2-(((6-((1S,2S)-2-(3-chlorophenyl)cyclopropane-1-carboxamido)pyrimidin-4-yl)amino)methyl)-5-cyclopropylpyrazolo[1,5-a]pyridin-7-yl)propanoate (20 mg, 0.034 mmol) in THF (1 mL) and H₂O (1 mL) was added LiOH—H₂O (14 mg, 0.34 mmol). The mixture was stirred at room temperature for 1 h. The mixture was quenched with HCl (1M aq., 1 mL) and concentrated in vacuo to give the crude product, which was purified by preparative TLC (eluent: DCM/MeOH=10/1) to give 3-(2-(((6-((1S,2S)-2-(3-chlorophenyl)cyclopropane-1-carboxamido)pyrimidin-4-yl)amino)methyl)-5-cyclopropylpyrazolo[1,5-a]pyridin-7-yl)propanoic acid (10.5 mg, 58%) as a white solid. ESI-MS [M+H]⁺: 531.2. ¹H NMR (400 MHz, DMSO) δ 10.60 (s, 1H), 8.20 (s, 1H), 7.91 (s, 1H), 7.37-7.08 (m, 6H), 6.43 (s, 1H), 6.31 (s, 1H), 4.64 (s, 2H), 3.25-3.23 (m, 2H), 2.79-2.67 (m, 2H), 2.41-2.38 (m, 2H), 1.92-1.91 (m, 1H), 1.47-1.40 (m, 2H), 0.97-0.96 (m, 2H), 0.74-0.73 (m, 2H).

### Example 49

### Synthesis of (1S,2S)-2-(3-chlorophenyl)-N-(6-(((5-cyclopropyl-7-(2-oxooxazolidin-3-yl)pyrazolo[1,5-a]pyridin-2-yl)methyl)amino)pyrimidin-4-yl)cyclopropane-1-carboxamide (I-49)

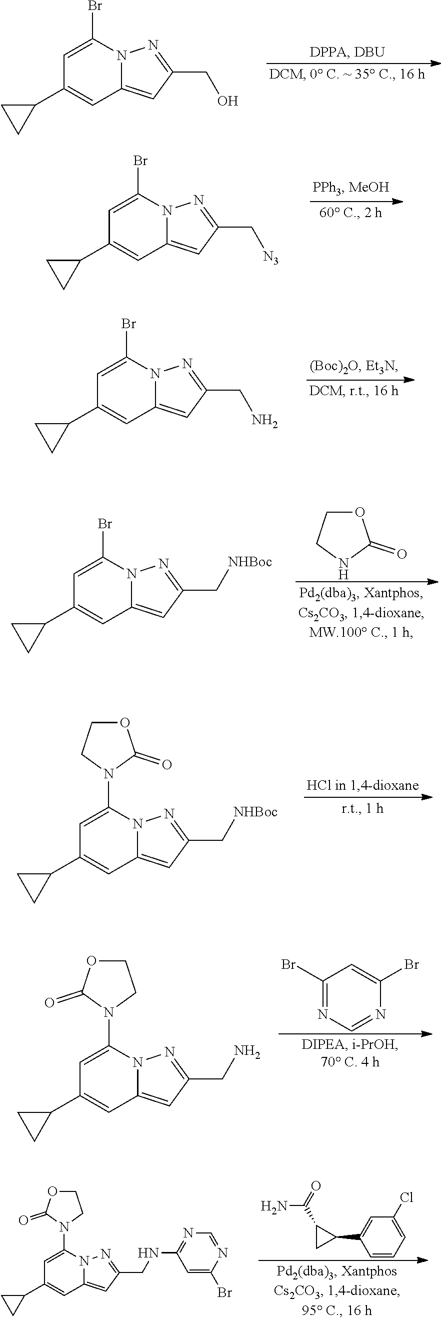

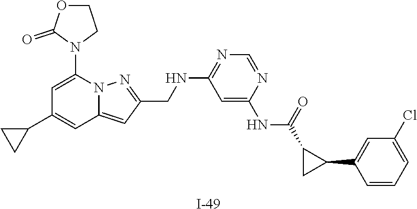

Synthesis of 2-(azidomethyl)-7-bromo-5-cyclopropylpyrazolo[1,5-a]pyridine. To a mixture of (7-bromo-5-cyclopropylpyrazolo[1,5-a]pyridin-2-yl)methanol (2 g, 7.5 mmol) in DCM (100 mL) was added DPPA (12.4 g, 45 mmol) and DBU (6.8 g, 45 mmol) at 0° C. under N₂. Then the mixture was stirred at 35° C. for 16 h. The reaction was quenched with water (100 mL) and extracted with DCM (50 mL×3). The combined organic layers were washed with brine (50 mL), dried over anhydrous Na₂SO₄, and concentrated in vacuo to give the crude product, which was purified by column chromatography eluting with a gradient of 0-10% EtOAc in PE to give 2-(azidomethyl)-7-bromo-5-cyclopropylpyrazolo[1,5-a]pyridine (2 g, 92%) as colorless oil. ESI-MS [M+H]⁺: 292.0.

Synthesis of (7-bromo-5-cyclopropylpyrazolo[1,5-a]pyridin-2-yl)methanamine. To a mixture of 2-(azidomethyl)-7-bromo-5-cyclopropylpyrazolo[1,5-a]pyridine (2 g, 6.9 mmol) in MeOH (40 mL) was added PPh₃ (2.7 g, 10.4 mmol). The reaction mixture was stirred at 60° C. for 2 h under N₂. The reaction mixture was concentrated in vacuo to give the crude, which was purified by column chromatography eluting with a gradient of 0-10% MeOH in DCM to give (7-bromo-5-cyclopropylpyrazolo[1,5-a]pyridin-2-yl)methanamine (1.2 g, 66%) as colorless oil. ESI-MS [M+H]⁺: 266.1.

Synthesis of tert-butyl ((7-bromo-5-cyclopropylpyrazolo[1,5-a]pyridin-2-yl)methyl)carbamate. A mixture of (7-bromo-5-cyclopropylpyrazolo[1,5-a]pyridin-2-yl)methanamine (1.2 g, 4.5 mmol), (Boc)₂O (1.96 g, 9.0 mmol) and Et₃N (1.36 g, 13.5 mmol) in DCM (40 mL) was stirred at room temperature for 16 h. The reaction mixture was diluted with H₂O (60 mL), extracted with DCM (40 mL×3). The combined organic layers were washed with brine (60 mL), dried over anhydrous Na₂SO₄ and concentrated in vacuo to give the crude, which was purified by silica gel column chromatography eluting with a gradient of 0-30% EtOAc in PE to give tert-butyl ((7-bromo-5-cyclopropylpyrazolo[1,5-a]pyridin-2-yl)methyl)carbamate (1.2 g, 73%) as a white solid. ESI-MS [M+H]⁺: 366.1.

Synthesis of tert-butyl ((5-cyclopropyl-7-(2-oxooxazolidin-3-yl)pyrazolo[1,5-a]pyridin-2-yl)methyl)carbamate. A mixture of tert-butyl ((7-bromo-5-cyclopropylpyrazolo[1,5-a]pyridin-2-yl)methyl)carbamate (75 mg, 0.21 mmol), oxazolidin-2-one (28 mg, 0.32 mmol), Pd₂(dba)₃ (18 mg, 0.02 mmol), Xantphos (23 mg, 0.04 mmol) and Cs₂CO₃ (205 mg, 0.63 mmol) in 1,4-dioxane (5 mL) was stirred in a sealed tube. After degassing with N₂ for 1 min, the reaction was irradiated in a microwave reactor at 100° C. for 1 h. The reaction mixture was cooled to room temperature, filtered through Celite® and the filter cake was washed with DCM/MeOH (10/1, 30 mL). The filtrate was concentrated in vacuo to give the crude, which was purified by preparative TLC (eluent: PE/EtOAc=1/1) to give tert-butyl ((5-cyclopropyl-7-(2-oxooxazolidin-3-yl)pyrazolo[1,5-a]pyridin-2-yl)methyl)carbamate (45 mg, 58%) as yellow oil. ESI-MS [M+H]⁺: 373.2.

Synthesis of 3-(2-(aminomethyl)-5-cyclopropylpyrazolo[1,5-a]pyridin-7-yl)oxazolidin-2-one. A mixture of tert-butyl ((5-cyclopropyl-7-(2-oxooxazolidin-3-yl)pyrazolo[1,5-a]pyridin-2-yl)methyl)carbamate (45 mg, 0.12 mmol) in HCl (4M solution in 1,4-dioxane, 2 mL) was stirred at room temperature for 1 h. The reaction mixture was quenched with saturated aqueous NaHCO₃ (30 mL) and extracted with DCM/MeOH (10/1, 20 mL×3). The combined organic layers were washed with water (30 mL), dried over anhydrous Na₂SO₄, and concentrated in vacuo to give (3-(2-(aminomethyl)-5-cyclopropylpyrazolo[1,5-a]pyridin-7-yl)oxazolidin-2-one (30 mg, 92%) as a yellow solid. ESI-MS [M+H]⁺: 273.2.

Synthesis of 3-(2-(((6-bromopyrimidin-4-yl)amino)methyl)-5-cyclopropylpyrazolo[1,5-a]pyridin-7-yl)oxazolidin-2-one. A mixture of 3-(2-(aminomethyl)-5-cyclopropylpyrazolo[1,5-a]pyridin-7-yl)oxazolidin-2-one (30 mg, 0.11 mmol), 4,6-dibromopyrimidine (40 mg, 0.17 mmol) and DIPEA (71 mg, 0.55 mmol) in i-PrOH (3 mL) was stirred at 70° C. for 4 h. The reaction mixture was concentrated in vacuo to give the crude, which was purified by preparative TLC (eluent: MeOH/DCM=1/10) to give 3-(2-(((6-bromopyrimidin-4-yl)amino)methyl)-5-cyclopropylpyrazolo[1,5-a]pyridin-7-yl)oxazolidin-2-one (30 mg, 64%) as a yellow solid. ESI-MS [M+H]⁺: 429.2.

Synthesis of (1S,2S)-2-(3-chlorophenyl)-N-(6-(((5-cyclopropyl-7-(2-oxooxazolidin-3-yl)pyrazolo[1,5-a]pyridin-2-yl)methyl)amino)pyrimidin-4-yl)cyclopropane-1-carboxamide. To a mixture of 3-(2-(((6-bromopyrimidin-4-yl)amino)methyl)-5-cyclopropylpyrazolo[1,5-a]pyridin-7-yl)oxazolidin-2-one (30 mg, 0.07 mmol), (1S,2S)-2-(3-chlorophenyl)cyclopropane-1-carboxamide (14 mg, 0.07 mmol) and Cs₂CO₃ (68 mg, 0.21 mmol) in 1,4-dioxane (4 mL) was added Pd₂(dba)₃ (13 mg, 0.014 mmol) and Xantphos (16 mg, 0.028 mmol). The mixture was stirred at 95° C. for 16 h under N₂. The reaction mixture was cooled to room temperature. The mixture was filtered through Celite® and the filter cake was washed with DCM/MeOH (10/1, 30 mL). The filtrate was concentrated in vacuo to give the crude, which was purified by preparative TLC (eluent: DCM/MeOH=20/1) to give (1S,2S)-2-(3-chlorophenyl)-N-(6-(((5-cyclopropyl-7-(2-oxooxazolidin-3-yl)pyrazolo[1,5-a]pyridin-2-yl)methyl)amino)pyrimidin-4-yl)cyclopropane-1-carboxamide (21 mg, 55%) as a white solid. ESI-MS [M+H]⁺: 544.2. ¹H NMR (400 MHz, DMSO) δ 10.61 (s, 1H), 8.20 (s, 1H), 7.93 (s, 1H), 7.35-7.30 (m, 3H), 7.27-7.25 (m, 2H), 7.15 (d, J=7.6 Hz, 1H), 6.73 (d, J=1.7 Hz, 1H), 6.40 (s, 1H), 4.66-4.57 (m, 4H), 4.22-4.18 (m, 2H), 2.46-2.29 (m, 2H), 2.03-1.96 (m, 1H), 1.52-1.37 (m, 2H), 1.06-0.96 (m, 2H), 0.81-0.73 (m, 2H).

### Example 50

### Synthesis of (S,2S)-2-(3-chlorophenyl)-N-(6-(((5-(cyclopropylamino)imidazo[1,2-c]pyrimidin-2-yl)methyl)amino)pyrimidin-4-cyclopropane-1-carboxamide (I-50)

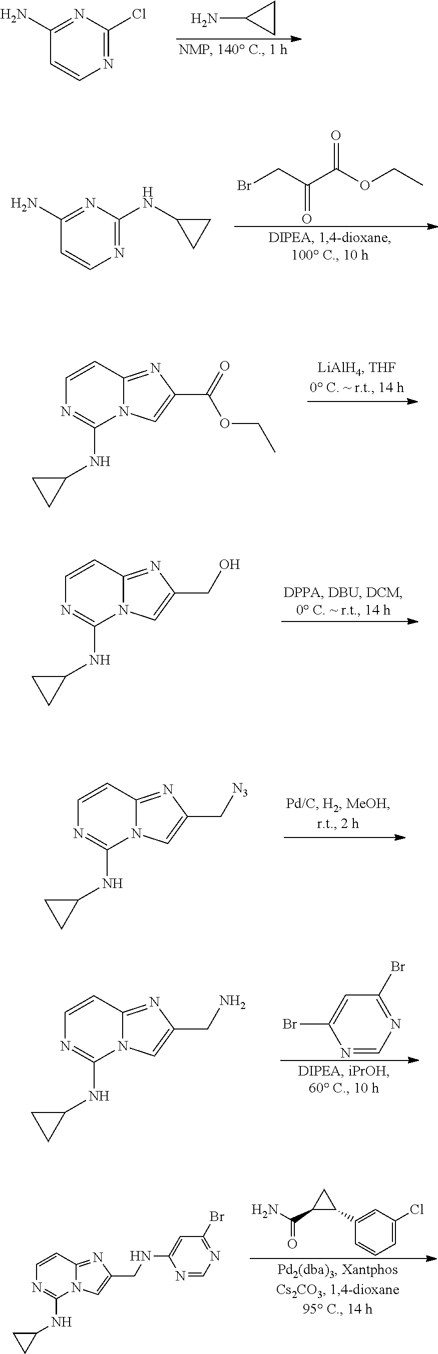

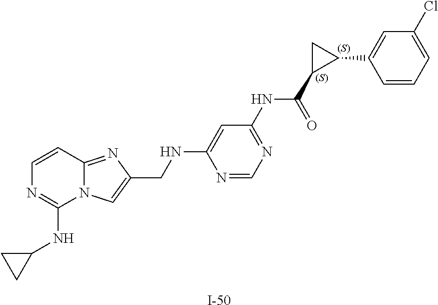

Synthesis of N₂-cyclopropylpyrimidine-2,4-diamine. A solution of 2-chloropyrimidin-4-amine (3.0 g, 23.2 mmol) and cyclopropanamine (6.6 L 116.3 mmol) in NMP (30 mL) in a sealed tube was stirred at 140° C. for 1 h. The reaction was cooled to room temperature, diluted with H₂O (80 mL), extracted with EtOAc (80 mL×3). The combined organic layers were washed with brine (80 mL), dried over anhydrous Na₂SO₄ then concentrated in vacuo to give the crude, which was purified with silica gel chromatography eluting with a gradient of 0-50% of EtOAc in PE to give N₂-cyclopropylpyrimidine-2,4-diamine (1.5 g, 43%) as a white solid. ESI-MS [M+H]⁺: 151.1

Synthesis of ethyl 5-(cyclopropylamino)imidazo[1,2-c]pyrimidine-2-carboxylate. To a solution of N₂-cyclopropylpyrimidine-2,4-diamine (1.5 g, 10 mmol) and DIPEA (6.5 g, 50 mmol) in 1,4-dioxane (50 mL) was added ethyl 3-bromo-2-oxopropanoate (4.9 g, 25 mmol). The resulting reaction was stirred at 100° C. for 10 h. The reaction was cooled to room temperature, quenched with H₂O (50 mL), extracted with EtOAc (60 mL×3). The combined organic layers were washed with brine (60 mL), dried over anhydrous Na₂SO₄ then concentrated in vacuo to give the crude, which was purified by silica gel chromatography eluting with a gradient of 0-60% EtOAc in PE to give ethyl 5-(cyclopropylamino)imidazo[1,2-c]pyrimidine-2-carboxylate (440 mg, 18%) as a yellow solid. ESI-MS [M+H]⁺: 247.1.

Synthesis of (5-(cyclopropylamino)imidazo[1,2-c]pyrimidin-2-yl)methanol. To a ethyl 5-(cyclopropylamino)imidazo[1,2-c]pyrimidine-2-carboxylate (440.0 mg, 1.8 mmol) in THF (30 mL) was added LiAlH₄ (205.2 mg, 5.4 mmol) at 0° C. slowly. The resulting reaction was stirred at room temperature for 14 h. The reaction was cooled to 0° C., quenched with NH₄Cl (sat. aq., 30 mL) slowly, extracted with EtOAc (40 mL×5). The combined organic layers were washed with brine (40 mL), dried over Na₂SO₄ then concentrated in vacuo to give (5-(cyclopropylamino)imidazo[1,2-c]pyrimidin-2-yl)methanol (500 mg, crude) as a yellow solid, which was used without further purification. ESI-MS [M+H]⁺: 205.2

Synthesis of 2-(azidomethyl)-N-cyclopropylimidazo[1,2-c]pyrimidin-5-amine. To a solution of (5-(cyclopropylamino)imidazo[1,2-c]pyrimidin-2-yl)methanol (500 mg, crude from previous step) and DBU (1.4 g, 8.9 mmol) in DCM (25 mL) was added DPPA (1.45 g, 5.3 mmol) at 0° C. The resulting reaction solution was stirred at room temperature for 14 h. The reaction was diluted with H₂O (50 mL), extracted with DCM (50 mL×3). The combine organic layers were washed with brine (40 mL), dried over anhydrous Na₂SO₄ and concentrated in vacuo to give the crude, which was purified with preparative TLC (eluent: DCM/MeOH=10/1) to give 2-(azidomethyl)-N-cyclopropylimidazo[1,2-c]pyrimidin-5-amine (210 mg, 51% over 2 steps) as a yellow solid. ESI-MS [M+H]⁺: 230.2.

Synthesis of 2-(aminomethyl)-N-cyclopropylimidazo[1,2-c]pyrimidin-5-amine. A mixture of 2-(azidomethyl)-N-cyclopropylimidazo[1,2-c]pyrimidin-5-amine (210 mg, 0.91 mmol) and Pd/C (30 mg) in MeOH (15 mL) was stirred at room temperature under a H₂ atmosphere for 2 h. The reaction mixture was filtered through Celite®, washed with MeOH (50 mL). The filtrate was concentrated in vacuo to give 2-(aminomethyl)-N-cyclopropylimidazo[1,2-c]pyrimidin-5-amine (195 mg, crude), which was used without further purification. ESI-MS [M+H]⁺: 203.2.

Synthesis of 2-(((6-bromopyrimidin-4-yl)amino)methyl)-N-cyclopropylimidazo[1,2-c]pyrimidin-5-amine. A 2-(aminomethyl)-N-cyclopropylimidazo[1,2-c]pyrimidin-5-amine (195 mg, crude), 4,6-dibromopyrimidine (237.8 mg, 1 mmol) and DIPEA (387 mg, 3 mmol) in iPrOH (20 mL) was stirred at 60° C. for 10 h. The reaction was concentrated in vacuo to give the crude, which was purified with preparative TLC (eluent: DCM/MeOH=10/1) to give 2-(((6-bromopyrimidin-4-yl)amino)methyl)-N-cyclopropylimidazo[1,2-c]pyrimidin-5-amine (250 mg, 76% over 2 steps) as a yellow solid. ESI-MS [M+H]⁺: 360.3.

Synthesis of (1S,2S)-2-(3-chlorophenyl)-N-(6-(((5-(cyclopropylamino)imidazo[1,2-c]pyrimidin-2-yl)methyl)amino)pyrimidin-4-yl)cyclopropane-1-carboxamide. A mixture of 2-(((6-bromopyrimidin-4-yl)amino)methyl)-N-cyclopropylimidazo[1,2-c]pyrimidin-5-amine (150 mg, 0.42 mmol), (1S,2S)-2-(3-chlorophenyl)cyclopropane-1-carboxamide (107.3 mg, 0.55 mmol), Pd₂(dba)₃ (57 mg, 0.063 mmol), Xantphos (69 mg, 0.12 mmol) and Cs₂CO₃ (410 mg, 1.26 mmol) in 1,4-dioxane (25 mL) was stirred at 95° C. for 14 h under N₂. The reaction was cooled to room temperature, diluted with H₂O (40 mL) then extracted with EtOAc (40 mL×3). The combined organic layers were washed with brine (40 mL), dried over Na₂SO₄ and concentrated in vacuo to give the crude, which was purified with preparative TLC (eluent: DCM/MeOH=10/1) to give (1S,2S)-2-(3-chlorophenyl)-N-(6-(((5-(cyclopropylamino)imidazo[1,2-c]pyrimidin-2-yl)methyl)amino)pyrimidin-4-yl)cyclopropane-1-carboxamide (20 mg, 10%) as a white solid. ESI-MS [M+H]⁺: 475.2. ¹H NMR (400 MHz, DMSO) δ 10.61 (s, 1H), 8.19 (s, 1H), 7.94 (s, 1H), 7.78 (s, 1H), 7.70 (s, 1H), 7.60 (d, J=6.4 Hz, 1H), 7.34-7.25 (m, 3H), 7.15 (d, J=7.6 Hz, 1H), 6.77 (d, J=6.4 Hz, 1H), 4.56 (s, 2H), 2.89-2.85 (m, 1H), 2.43-2.25 (m, J=12.6 Hz, 2H), 1.54-1.43 (m, J=4.1 Hz, 1H), 1.44-1.31 (m, 1H), 0.85-0.73 (m, 2H), 0.60-0.56 (m, 2H).

### Example 51

### Synthesis of (S,2S)-2-(3-chlorophenyl)-N-(6-(((5-cyclopropyl-7-(3-hydroxy-3-methylbutyl)pyrazolo[1,5-a]pyridin-2-yl)methyl)amino)pyrimidin-4-yl)cyclopropane-1-carboxamide (I-51)

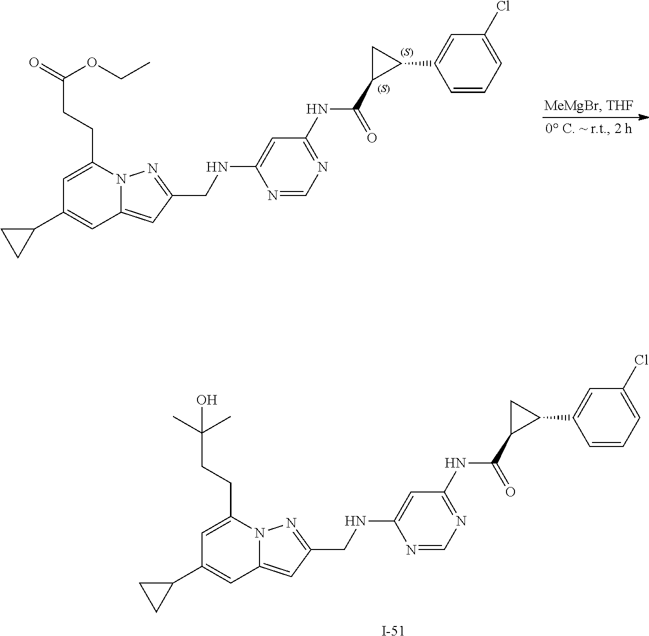

To a mixture of ethyl 3-(2-(((6-((1S,2S)-2-(3-chlorophenyl)cyclopropane-1-carboxamido)pyrimidin-4-yl)amino)methyl)-5-cyclopropylpyrazolo[1,5-a]pyridin-7-yl)propanoate (35 mg, 0.063 mmol) in THF (2 mL) was added MeMgBr (3 M solution in ether, 0.21 mL, 0.63 mmol) at 0° C. under N₂. The mixture was stirred at room temperature for 2 h under N₂. The reaction was quenched with NH₄Cl (sat. aq., 30 mL) and extracted with DCM (20 mL×3). The combined organic layers were washed with brine (30 mL), dried over anhydrous Na₂SO₄, and concentrated in vacuo to give the crude product, which was purified by preparative TLC (eluent: DCM/MeOH=15/1) to give (1S,2S)-2-(3-chlorophenyl)-N-(6-(((5-cyclopropyl-7-(3-hydroxy-3-methylbutyl)pyrazolo[1,5-a]pyridin-2-yl)methyl)amino)pyrimidin-4-yl)cyclopropane-1-carboxamide (6.5 mg, 19%) as a white solid. ESI-MS [M+H]⁺: 545.2. ¹H NMR (400 MHz, DMSO) δ 10.60 (s, 1H), 8.20 (s, 1H), 7.89 (s, 1H), 7.34-7.24 (m, 4H), 7.17-7.14 (m, 2H), 6.40 (s, 1H), 6.29 (s, 1H), 4.64 (s, 2H), 4.34 (s, 1H), 3.11-3.02 (m, 2H), 2.44-2.35 (m, 2H), 1.94-1.92 (m, 1H), 1.82-1.80 (m, 2H), 1.49-1.38 (m, 2H), 1.19 (s, 6H), 0.99-0.93 (m, 2H), 0.74-0.73 (m, 2H).

### Example 52

### Synthesis of (1S,2S)-2-(3-chlorophenyl)-N-(6-(((5-cyclopropyl-7-(2-oxopyrrolidin-1-yl)pyrazolo[1,5-a]pyridin-2-yl)methyl)amino)pyrimidin-4-yl)cyclopropane-1-carboxamide (I-52)

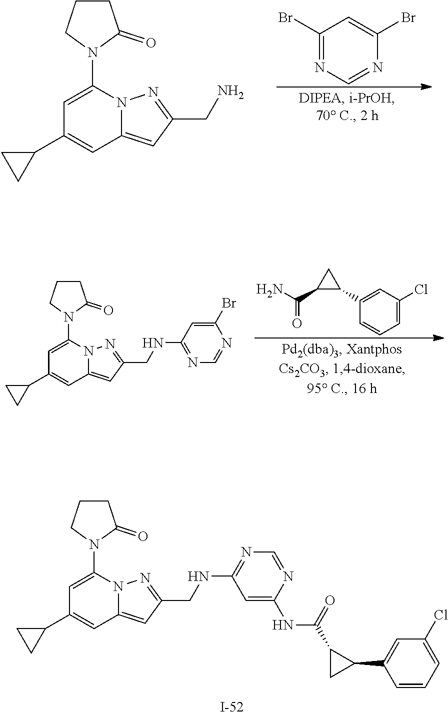

Synthesis of 1-(2-(((6-bromopyrimidin-4-yl)amino)methyl)-5-cyclopropylpyrazolo[1,5-a]pyridin-7-yl)pyrrolidin-2-one. A mixture of 1-(2-(aminomethyl)-5-cyclopropylpyrazolo[1,5-a]pyridin-7-yl)pyrrolidin-2-one (40 mg, 0.15 mmol), 4,6-dibromopyrimidine (71 mg, 0.3 mmol) and DIPEA (97 mg, 0.75 mmol) in i-PrOH (4 mL) was stirred at 70° C. for 2 h. The reaction mixture was concentrated in vacuo to give the crude, which was purified by preparative TLC (eluent: MeOH/DCM=1/10) to give 1-(2-(((6-bromopyrimidin-4-yl)amino)methyl)-5-cyclopropylpyrazolo[1,5-a]pyridin-7-yl)pyrrolidin-2-one (35 mg, 55%) as a yellow solid. ESI-MS [M+H]⁺: 427.2.

Synthesis of (1S,2S)-2-(3-chlorophenyl)-N-(6-(((5-cyclopropyl-7-(2-oxopyrrolidin-1-yl)pyrazolo[1,5-a]pyridin-2-yl)methyl)amino)pyrimidin-4-yl)cyclopropane-1-carboxamide. To a mixture of 1-(2-(((6-bromopyrimidin-4-yl)amino)methyl)-5-cyclopropylpyrazolo[1,5-a]pyridin-7-yl)pyrrolidin-2-one (35 mg, 0.08 mmol), (1S,2S)-2-(3-chlorophenyl)cyclopropane-1-carboxamide (16 mg, 0.08 mmol) and Cs₂CO₃ (78 mg, 0.24 mmol) in 1,4-dioxane (3 mL) was added Pd₂(dba)₃ (15 mg, 0.016 mmol) and Xantphos (9 mg, 0.016 mmol). The mixture was stirred at 95° C. for 16 h under N₂. The reaction mixture was cooled to room temperature. The mixture was filtered through Celite® and the filter cake was washed with DCM/MeOH (10/1, 30 mL). The filtrate was concentrated in vacuo to give the crude, which was purified by preparative TLC (eluent: DCM/MeOH=20/1) to give (1S,2S)-2-(3-chlorophenyl)-N-(6-(((5-cyclopropyl-7-(2-oxopyrrolidin-1-yl)pyrazolo[1,5-a]pyridin-2-yl)methyl)amino)pyrimidin-4-yl)cyclopropane-1-carboxamide (4.4 mg, 10%) as a white solid. ESI-MS [M+H]⁺: 542.2. ¹H NMR (400 MHz, DMSO) δ 10.58 (s, 1H), 8.16 (s, 1H), 7.88 (s, 1H), 7.32-7.22 (m, 5H), 7.12 (d, J=7.6 Hz, 1H), 6.55 (d, J=1.8 Hz, 1H), 6.34 (s, 1H), 4.60 (s, 2H), 3.89 (t, J=7.0 Hz, 2H), 2.49-2.47 (m, 2H), 2.36-2.30 (m, 2H), 2.24-2.09 (m, 2H), 1.98-1.91 (m, 1H), 1.45-1.35 (m, 2H), 1.04-0.91 (m, 2H), 0.83-0.66 (m, 2H).

### Example 53

### Synthesis of (1S,2S)-2-(3-chlorophenyl)-N-(6-(((5-cyclopropyl-7-(4-methylpiperazin-1-yl)pyrazolo[1,5-a]pyridin-2-yl)methyl)amino)pyrimidin-4-yl)cyclopropane-1-carboxamide (I-53)

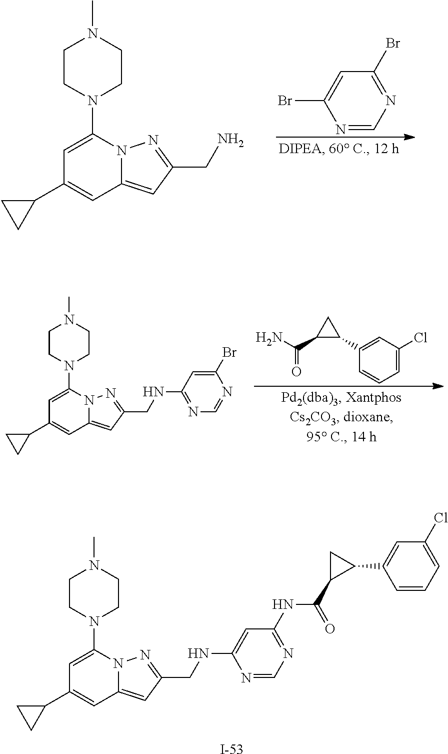

Synthesis of 6-bromo-N-((5-cyclopropyl-7-(4-methylpiperazin-1-yl)pyrazolo[1,5-a]pyridin-2-yl)methyl)pyrimidin-4-amine. A mixture of (5-cyclopropyl-7-(4-methylpiperazin-1-yl)pyrazolo[1,5-a]pyridin-2-yl)methanamine hydrochloride (80 mg, 0.25 mmol), 4,6-dibromopyrimidine (88 mg, 0.37 mmol), DIPEA (97 mg, 0.75 mmol) in i-PrOH (4 ml) was stirred at 60° C. for 12 h. After cooling to room temperature, the reaction was quenched with water (30 mL) and extracted with DCM (30 mL×3). The combined organic layers were washed with brine (30 mL), dried over Na₂SO₄ and concentrated in vacuo to give the crude, which was purified by Pre-TLC (eluent: DCM/MeOH=25/1) to give 6-bromo-N-((5-cyclopropyl-7-(4-methylpiperazin-1-yl)pyrazolo[1,5-a]pyridin-2-yl)methyl)pyrimidin-4-amine (59 mg, 53%) as a yellow solid. ESI-MS [M+H]⁺: 442.2.

Synthesis of (1S,2S)-2-(3-chlorophenyl)-N-(6-(((5-cyclopropyl-7-(4-methylpiperazin-1-yl)pyrazolo[1,5-a]pyridin-2-yl)methyl)amino)pyrimidin-4-yl)cyclopropane-1-carboxamide. To a mixture of 2-bromo-N-((5-cyclopropyl-7-(4-methylpiperazin-1-yl)pyrazolo[1,5-a]pyridin-2-yl)methyl)pyridin-4-amine (59 mg, 0.13 mmol), (1S,2S)-2-(3-chlorophenyl)cyclopropane-carboxamide (25 mg, 0.13 mmol) and Cs₂CO₃ (127 mg, 0.39 mmol) in 1,4-dioxane (5 mL) was added Pd₂(dba)₃ (18 mg, 0.02 mmol), xantphos (21 mg, 0.036 mmol), The mixture was stirred at 95° C. for 6 h under N₂. After cooling to room temperature, the reaction mixture was filtered through Celite® and the filter cake was washed with DCM/MeOH (10/1, 100 mL). The filtrate was concentrated in vacuo to give the crude, which was purified by preparative TLC (eluent: DCM/MeOH=20/1) to give (1S,2S)-2-(3-chlorophenyl)-N-(6-(((5-cyclopropyl-7-(4-methyl piperazin-1-yl)pyrazolo[1,5-a]pyridin-2-yl)methyl)amino)pyrimidin-4-yl)cyclopropane-1-carboxamide (13 mg, 18%) as a white solid. ESI-MS [M+H]⁺: 557.2, ¹H NMR (400 MHz, DMSO) δ 10.60 (s, 1H), 8.19 (s, 1H), 7.85 (s, 1H), 7.32-7.20 (m, 4H), 7.14 (d, J=7.6 Hz, 1H), 6.91 (d, J=1.4 Hz, 1H), 6.24 (s, 1H), 5.92 (d, J=1.3 Hz, 1H), 4.63 (s, 2H), 3.34-3.33 (m, 4H), 2.51-2.50 (m, 4H), 2.42-2.35 (m, 2H), 2.25 (s, 3H), 1.97-1.86 (m, 1H), 1.51-1.35 (m, 2H), 0.99-0.90 (m, 2H), 0.80-0.70 (m, 2H).

### Example 54

### Synthesis of (1S,2S)-2-(3-chlorophenyl)-N-(4-(((5-cyclopropyl-7-(4-methylpiperazin-1-yl)pyrazolo[1,5-a]pyridin-2-yl methyl)amino)pyridin-2-yl cyclopropane-1-carboxamide (I-54)

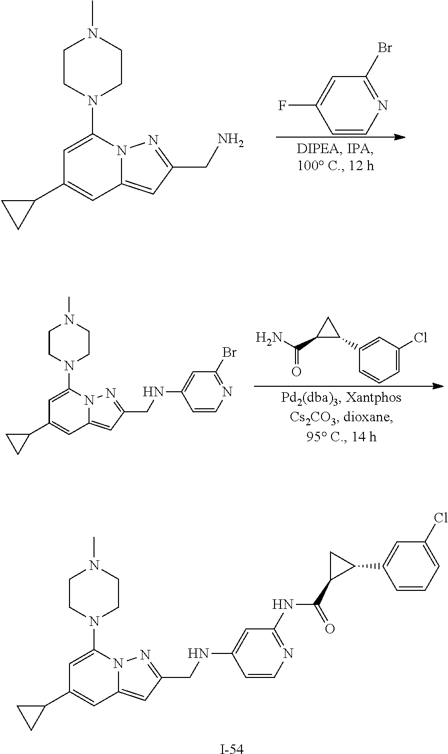

Synthesis of 2-bromo-N-((5-cyclopropyl-7-(4-methylpiperazin-1-yl)pyrazolo[1,5-a]pyridin-2-yl)methyl)pyridin-4-amine. A mixture of (5-cyclopropyl-7-(4-methylpiperazin-1-yl)pyrazolo[1,5-a]pyridin-2-yl)methanamine hydrochloride (100 mg, 0.31 mmol), 2-bromo-4-fluoropyridine (70 mg, 0.40 mmol), DIPEA (120 mg, 0.93 mmol) in i-PrOH (4 ml) was stirred at 100° C. for 12 h. After cooling to room temperature, the reaction was quenched with NaHCO₃ (sat. aq., 30 mL) and extracted with DCM (30 mL×3). The combined organic layers were washed with brine (30 mL), dried over Na₂SO₄ and concentrated in vacuo to give the crude, which was purified by preparative TLC (eluent: DCM/MeOH=25/1) to give 6-bromo-N-((5-cyclopropyl-7-(4-methyl piperazin-1-yl)pyrazolo[1,5-a]pyridin-2-yl)methyl)pyrimidin-4-amine (50 mg, 37%) as a yellow solid. ESI-MS [M+H]⁺: 441.2.

Synthesis of (1S,2S)-2-(3-chlorophenyl)-N-(4-(((5-cyclopropyl-7-(4-methylpiperazin-1-yl)pyrazolo[1,5-a]pyridin-2-yl)methyl)amino)pyridin-2-yl)cyclopropane-1-carboxamide. To a mixture of 2-bromo-N-((5-cyclopropyl-7-(4-methylpiperazin-1-yl)pyrazolo[1,5-a]pyridin-2-yl)methyl)pyridin-4-amine (59 mg, 0.13 mmol), (1S,2S)-2-(3-chlorophenyl)cyclopropane-carboxamide (25 mg, 0.13 mmol) and Cs₂CO₃ (127 mg, 0.39 mmol) in 1,4-dioxane (5 mL) was added Pd₂(dba)₃ (18 mg, 0.02 mmol), Xantphos (21 mg, 0.036 mmol), The mixture was stirred at 95° C. for 6 h under N₂. After cooling to room temperature, the reaction mixture was filtered through Celite® and the filter cake was washed with DCM/MeOH (10/1, 100 mL). The filtrate was concentrated in vacuo to give the crude, which was purified by preparative TLC (eluent: DCM/MeOH=20/1) to give (1S,2S)-2-(3-chlorophenyl)-N-(4-(((5-cyclopropyl-7-(4-methylpiperazin-1-yl)pyrazolo[1,5-a]pyridin-2-yl)methyl)amino)pyridin-2-yl)cyclopropane-1-carboxamide (13 mg, 18%) as a white solid. ESI-MS [M+H]⁺: 556.2, ¹H NMR (400 MHz, DMSO) δ 10.30 (s, 1H), 7.75 (d, J=5.8 Hz, 1H), 7.46 (s, 1H), 7.34-7.18 (m, 1H), 7.27-7.20 (m, 2H), 7.17-7.11 (m, 2H), 6.92 (d, J=1.4 Hz, 1H), 6.36-6.30 (m, 1H), 6.26 (s, 1H), 5.94 (d, J=1.5 Hz, 1H), 4.41 (d, J=5.9 Hz, 2H), 3.34-3.33 (m, 4H), 2.51-2.50 (m, 4H), 2.42-2.31 (m, 2H), 2.26 (s, 3H), 1.98-1.88 (m, 1H), 1.49-1.39 (m, 1H), 1.39-1.30 (m, 1H), 0.97-0.89 (m, 2H), 0.80-0.70 (m, 2H).

### Example 55

### Synthesis of (1S,2S)-2-(3-chlorophenyl)-N-(6-(((7-(2-cyano-2-methylpropyl)-5-cyclopropylpyrazolo[1,5-a]pyridin-2-yl)methyl)amino)pyrimidin-4-yl)cyclopropane-1-carboxamide (I-55)

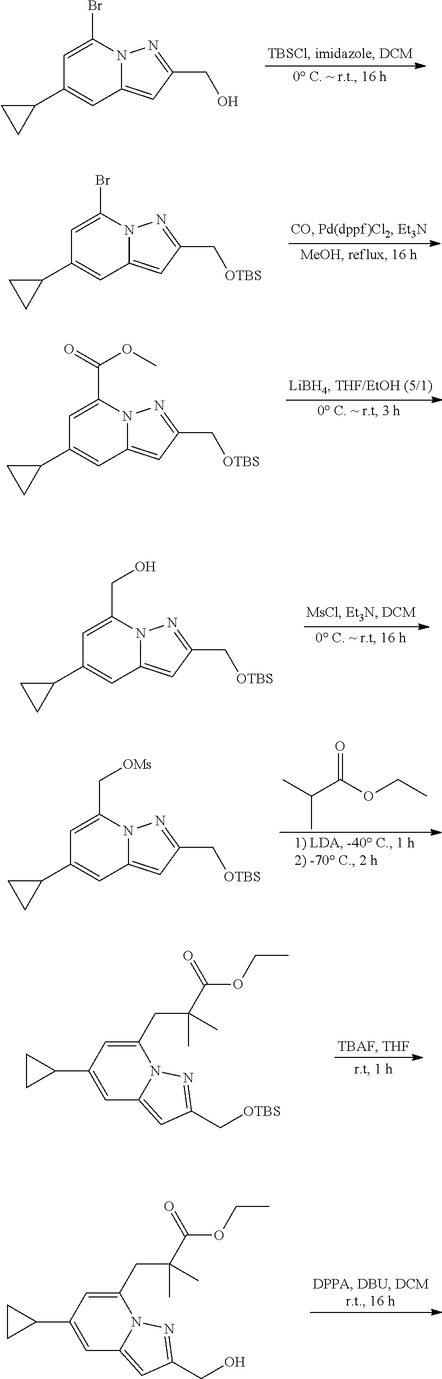

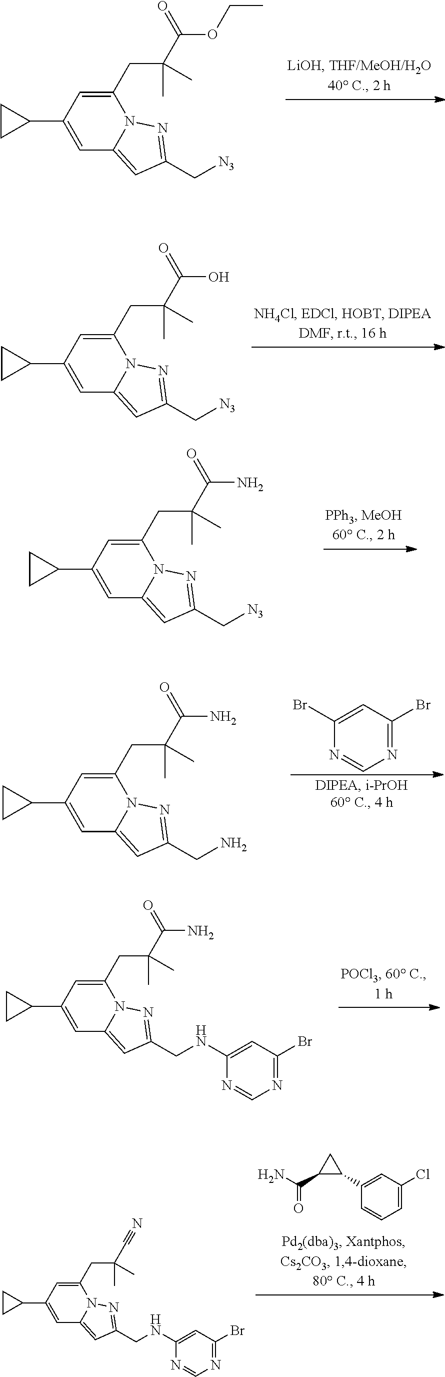

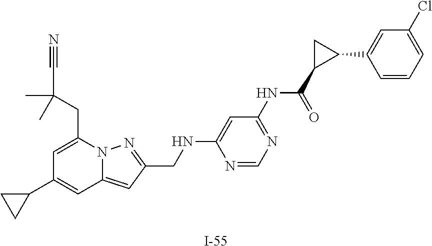

Synthesis of 7-bromo-2-(((tert-butyldimethylsilyl)oxy)methyl)-5-cyclopropylpyrazolo[1,5-a]pyridine. To a stirred solution of (7-bromo-5-cyclopropylpyrazolo[1,5-a]pyridin-2-yl)methanol (2.8 g, 10.48 mmol) and imidazole (2.14 g, 31.44 mmol) in DCM (30 mL) was added dropwise a solution of TBSCl (1.90 g, 12.58 mmol) at 0° C. The mixture was stirred at room temperature for 16 h then diluted with DCM (100 mL) and washed with water (80 mL) and brine (80 mL). The organics were dried over Na₂SO₄, concentrated in vacuo and purified by silica gel chromatography (EA/PE=1/3) to give 7-bromo-2-(((tert-butyldimethylsilyl)oxy)methyl)-5-cyclopropylpyrazolo[1,5-a]pyridine (3.69 g, 92%) as a white solid. ESI-MS [M+H]⁺: 381.0.

Synthesis of methyl 2-(((tert-butyldimethylsilyl)oxy)methyl)-5-cyclopropylpyrazolo[1,5-a]pyridine-7-carboxylate. A mixture of 7-bromo-2-(((tert-butyldimethylsilyl)oxy)methyl)-5-cyclopropylpyrazolo[1,5-a]pyridine (2.8 g, 7.34 mmol), Pd(dppf)C12 (537 mg, 0.73 mmol) and Et₃N (3.71 g, 36.7 mmol) in MeOH (40 mL) was stirred at reflux for 16 h under CO (balloon). After the reaction was cooled to room temperature, the reaction mixture was diluted in MeOH (50 mL) and filtered. The filtrate was concentrated in vacuo and purified by silica gel chromatography (EA/PE=1/20) to give methyl 2-(((tert-butyldimethylsilyl)oxy)methyl)-5-cyclopropylpyrazolo[1,5-a]pyridine-7-carboxylate (1.08 g, 41%) as a colorless syrup. ESI-MS [M+H]⁺: 361.2.

Synthesis of (2-(((tert-butyldimethylsilyl)oxy)methyl)-5-cyclopropylpyrazolo[1,5-a]pyridin-7-yl)methanol. To a stirred solution of methyl 2-(((tert-butyldimethylsilyl)oxy)methyl)-5-cyclopropylpyrazolo[1,5-a]pyridine-7-carboxylate (1.08 g, 3.0 mmol) in THF (30 mL) and EtOH (6 mL) was added LiBH₄ (196 mg, 9.0 mmol) in portions at 0° C. The mixture was stirred at room temperature for 16 h. The reaction mixture was quenched with saturated NH₄Cl aqueous (40 mL) and extracted with EtOAc (40 ml×2). The combined organics were washed with brine (80 mL), dried over Na₂SO₄, concentrated in vacuo and purified by silica gel chromatography (EA/PE=1/5) to give (2-(((tert-butyldimethylsilyl)oxy)methyl)-5-cyclopropylpyrazolo[1,5-a]pyridin-7-yl)methanol (760 mg, 76%) as a colorless syrup. ESI-MS [M+H]⁺: 333.2.

Synthesis of (2-(((tert-butyldimethylsilyl)oxy)methyl)-5-cyclopropylpyrazolo[1,5-a]pyridin-7-yl)methyl methanesulfonate. To a mixture of (2-(((tert-butyldimethylsilyl)oxy)methyl)-5-cyclopropylpyrazolo[1,5-a]pyridin-7-yl)methanol (760 mg, 2.29 mmol) and Et₃N (1.39 g, 13.74 mmol) in DCM (20 mL) was added dropwise a solution of MsCl (393 mg, 3.44 mmol) in DCM (5 mL) at 0° C. and stirred at room temperature for 3 h. The reaction mixture was diluted in DCM (100 mL) and washed with water (50 mL) then brine (50 mL) and dried over Na₂SO₄. The organic layer was concentrated and dried in vacuo to give (2-(((tert-butyldimethylsilyl)oxy)methyl)-5-cyclopropylpyrazolo[1,5-a]pyridin-7-yl)methyl methanesulfonate (840 mg, 89%) as a yellow syrup. ESI-MS [M+H]⁺: 411.1.

Synthesis of ethyl 3-(2-(((tert-butyldimethylsilyl)oxy)methyl)-5-cyclopropylpyrazolo[1,5-a]pyridin-7-yl)-2,2-dimethylpropanoate. To a stirred solution of ethyl isobutyrate (714 mg, 6.15 mmol) in THF (20 mL) was added LDA (3.08 mL, 6.15 mmol, 2M in THF) dropwise at −40° C. under N₂. After the reaction was stirred at this temperature for 1 h, the reaction was cooled to −70° C. and a solution of (2-(((tert-butyldimethylsilyl)oxy)methyl)-5-cyclopropylpyrazolo[1,5-a]pyridin-7-yl)methyl methanesulfonate (840 mg, 2.05 mmol) in THF (5 mL) was added dropwise at −70° C. over 20 min. The resulting mixture was stirred for 1.5 h at −70° C. The reaction mixture was then quenched with NH₄Cl (aq., 30 mL), extracted with EtOAc (40 mL×3). The combined organics were washed with brine (80 mL), dried over Na₂SO₄, concentrated in vacuo and purified by silica gel chromatography (EtOAc/PE=1/10) to give ethyl 3-(2-(((tert-butyldimethylsilyl)oxy)methyl)-5-cyclopropylpyrazolo[1,5-a]pyridin-7-yl)-2,2-dimethylpropanoate (260 mg, 29%) as a colorless syrup. ESI-MS [M+H]⁺: 431.2.

Synthesis of ethyl 3-(5-cyclopropyl-2-(hydroxymethyl)pyrazolo[1,5-a]pyridin-7-yl)-2,2-dimethylpropanoate. To a solution of ethyl 3-(2-(((tert-butyldimethylsilyl)oxy)methyl)-5-cyclopropylpyrazolo[1,5-a]pyridin-7-yl)-2,2-dimethylpropanoate (260 mg, 0.60 mmol) in THF (6 mL) was added TBAF (1 mL, 1 M in THF) and the reaction was stirred at room temperature for 1 h. The reaction mixture was diluted in water (30 mL) and extracted with EtOAc (20 ml×3). The combined organics were washed with brine (40 mL), dried over Na₂SO₄, concentrated in vacuo and purified by silica gel chromatography (EA/PE=1/2) to give ethyl 3-(5-cyclopropyl-2-(hydroxymethyl)pyrazolo[1,5-a]pyridin-7-yl)-2,2-dimethylpropanoate (160 mg, 84%) as a yellow syrup. ESI-MS [M+H]⁺: 317.2.

Synthesis of ethyl 3-(2-(azidomethyl)-5-cyclopropylpyrazolo[1,5-a]pyridin-7-yl)-2,2-dimethylpropanoate. To a solution of ethyl 3-(5-cyclopropyl-2-(hydroxymethyl)pyrazolo[1,5-a]pyridin-7-yl)-2,2-dimethylpropanoate (410 mg, 1.3 mmol) and DPPA (2.1 g, 7.8 mmol) in toluene (12 mL) was added dropwise a solution of DBU (1.2 g, 7.8 mmol) in toluene (3.0 mL) at 0° C. and stirred at room temperature for 16 h. The reaction mixture was diluted in water (50 mL) and extracted with EtOAc (50 ml×3). The combined organics were washed with brine (50 mL), dried over Na₂SO₄, concentrated in vacuo to give the crude product, which was purified by column chromatography (eluent: EtOAc/PE=1/4) to give ethyl 3-(2-(azidomethyl)-5-cyclopropylpyrazolo[1,5-a]pyridin-7-yl)-2,2-dimethylpropanoate (430 mg, 97%) as a yellow syrup. ESI-MS [M+H]⁺: 342.2.

Synthesis of 3-(2-(azidomethyl)-5-cyclopropylpyrazolo[1,5-a]pyridin-7-yl)-2,2-dimethylpropanoic acid. A mixture of ethyl 3-(2-(azidomethyl)-5-cyclopropylpyrazolo[1,5-a]pyridin-7-yl)-2,2-dimethylpropanoate (400 mg, 1.2 mmol) and LiOH—H₂O (200 mg, 4.8 mmol) in THF (4.0 mL), MeOH (4.0 mL) and H₂O (3.0 mL) was stirred at 40° C. for 4 h. After cooling to room temperature, the mixture was diluted with water (30 mL), acidified to pH 4-5 with HCl (3 M aq.) and extracted with EtOAc (40 mL×2). The combined organics were washed with brine (80 mL), dried over Na₂SO₄, concentrated and dried in vacuo to give 3-(2-(azidomethyl)-5-cyclopropylpyrazolo[1,5-a]pyridin-7-yl)-2,2-dimethylpropanoic acid (370 mg, 98%) as a white syrup. ESI-MS [M+H]⁺: 314.1.

Synthesis of 3-(2-(azidomethyl)-5-cyclopropylpyrazolo[1,5-a]pyridin-7-yl)-2,2-dimethylpropanamide. A mixture of 3-(2-(azidomethyl)-5-cyclopropylpyrazolo[1,5-a]pyridin-7-yl)-2,2-dimethylpropanoic acid (370 mg, 1.2 mmol), NH₄Cl (636 mg, 12 mmol), EDCI (688 mg, 3.6 mmol), HOBt (486 mg, 3.6 mmol) and DIPEA (929 mg, 7.2 mmol) in DMF (10 mL) was stirred at room temperature for 16 h. The reaction mixture was poured into water (100 mL) and extracted with EtOAc (50 mL×3). The combined organics were washed with brine (30 mL), dried over Na₂SO₄ then concentrated and dried in vacuo to give 3-(2-(azidomethyl)-5-cyclopropylpyrazolo[1,5-a]pyridin-7-yl)-2,2-dimethylpropanamide (330 mg, 88%) as a white solid. ESI-MS [M+H]⁺: 313.2.

Synthesis of 3-(2-(aminomethyl)-5-cyclopropylpyrazolo[1,5-a]pyridin-7-yl)-2,2-dimethylpropanamide. A mixture of 3-(2-(azidomethyl)-5-cyclopropylpyrazolo[1,5-a]pyridin-7-yl)-2,2-dimethylpropanamide (330 mg, 1.1 mmol) and PPh₃ (865 mg, 3.3 mmol) in MeOH (10 mL) was stirred at 60° C. for 2 h. After cooling to room temperature, the mixture was concentrated in vacuo to give the crude product, which was purified by column chromatography (eluent: DCM/MeOH=5/1) to give 3-(2-(aminomethyl)-5-cyclopropylpyrazolo[1,5-a]pyridin-7-yl)-2,2-dimethylpropanamide (280 mg, 89%) as a yellow solid. ESI-MS [M+H]⁺: 287.2.

Synthesis of 3-(2-(((6-bromopyrimidin-4-yl)amino)methyl)-5-cyclopropylpyrazolo[1,5-a]pyridin-7-yl)-2,2-dimethylpropanamide. A mixture of 3-(2-(aminomethyl)-5-cyclopropylpyrazolo[1,5-a]pyridin-7-yl)-2,2-dimethylpropanamide (260 mg, 0.91 mmol), 4,6-dibromopyrimidine (330 mg, 1.4 mmol) and DIPEA (580 mg, 4.5 mmol) in i-PrOH (6.0 mL) was stirred at 60° C. for 4 h. After cooling to room temperature, the reaction was quenched with NaHCO₃ (sat. aq., 30 mL) and extracted with DCM (30 mL×3). The combined organic layers were washed with brine (30 mL), dried over Na₂SO₄ then concentrated in vacuo to give the crude, which was purified by column chromatography (eluent: EtOAc/PE=1/1) to give 3-(2-(((6-bromopyrimidin-4-yl)amino)methyl)-5-cyclopropylpyrazolo[1,5-a]pyridin-7-yl)-2,2-dimethylpropanamide (360 mg, 90%) as a yellow solid. ESI-MS [M+H]⁺: 443.0.

Synthesis of 3-(2-(((6-bromopyrimidin-4-yl)amino)methyl)-5-cyclopropylpyrazolo[1,5-a]pyridin-7-yl)-2,2-dimethylpropanenitrile. The mixture of 3-(2-(((6-bromopyrimidin-4-yl)amino)methyl)-5-cyclopropylpyrazolo[1,5-a]pyridin-7-yl)-2,2-dimethylpropanamide (360 mg, 0.81 mmol) in POCl₃ (5.0 mL) was stirred at 60° C. for 1 h. After cooling to room temperature, the reaction mixture was added dropwise to a stirred NaHCO₃ (sat. aq., 50 mL) and extracted with EtOAc (40 mL×3). The combined organics were washed with brine (40 mL), dried over Na₂SO₄, concentrated in vacuo to give the crude product, which was purified by column chromatography (eluent: EtOAc/PE=1/1) to give 3-(2-(((6-bromopyrimidin-4-yl)amino)methyl)-5-cyclopropylpyrazolo[1,5-a]pyridin-7-yl)-2,2-dimethylpropanenitrile (240 mg, 70%) as a yellow solid. ESI-MS [M+H]⁺: 425.1.

Synthesis of (1S,2S)-2-(3-chlorophenyl)-N-(6-(((7-(2-cyano-2-methylpropyl)-5-cyclopropylpyrazolo[1,5-a]pyridin-2-yl)methyl)amino)pyrimidin-4-yl)cyclopropane-1-carboxamide. A mixture of 3-(2-(((6-bromopyrimidin-4-yl)amino)methyl)-5-cyclopropylpyrazolo[1,5-a]pyridin-7-yl)-2,2-dimethylpropanenitrile (120 mg, 0.28 mmol), (1S,2S)-2-(3-chlorophenyl)cyclopropane-1-carboxamide (55 mg, 0.28 mmol), Pd₂(dba)₃ (26 mg, 0.028 mmol), Xantphos (32 mg, 0.056 mmol) and Cs₂CO₃ (274 mg, 0.84 mmol) in 1,4-dioxane (8.0 mL) was stirred at 80° C. for 4 h under nitrogen. After cooling to room temperature, the reaction mixture was filtered through Celite® and the filter cake was washed with DCM/MeOH (10/1, 100 mL). The filtrate was concentrated in vacuo to give the crude, which was purified by column chromatography (eluent: EtOAc/PE=2/1) and preparative TLC (eluent: EtOAc) to give (1S,2S)-2-(3-chlorophenyl)-N-(6-(((7-(2-cyano-2-methylpropyl)-5-cyclopropylpyrazolo[1,5-a]pyridin-2-yl)methyl)amino)pyrimidin-4-yl)cyclopropane-1-carboxamide (70 mg, 46%) as a white solid. ESI-MS [M+H]⁺: 540.2. ¹H NMR (400 MHz, DMSO) δ 10.60 (s, 1H), 8.19 (s, 1H), 7.86 (s, 1H), 7.35-7.23 (m, 5H), 7.15 (d, J=7.6 Hz, 1H), 6.52 (d, J=1.8 Hz, 1H), 6.32 (s, 1H), 4.63 (s, 2H), 3.46 (s, 2H), 2.44-2.34 (m, 2H), 2.00-1.92 (m, 1H), 1.50-1.44 (m, 1H), 1.43-1.38 (m, 1H), 1.35 (s, 6H), 1.04-0.96 (m, 2H), 0.79-0.71 (m, 2H).

### Example 56

### Synthesis of rac-(1S*,2S*)-2-(4-chloropyridin-2-yl)-N-(6-(((5-cyclopropyl-7-(2-oxooxazolidin-3-yl)pyrazolo[1,5-a]pyridin-2-yl)methyl)amino)pyrimidin-4-yl)cyclopropane-1-carboxamide (I-56)

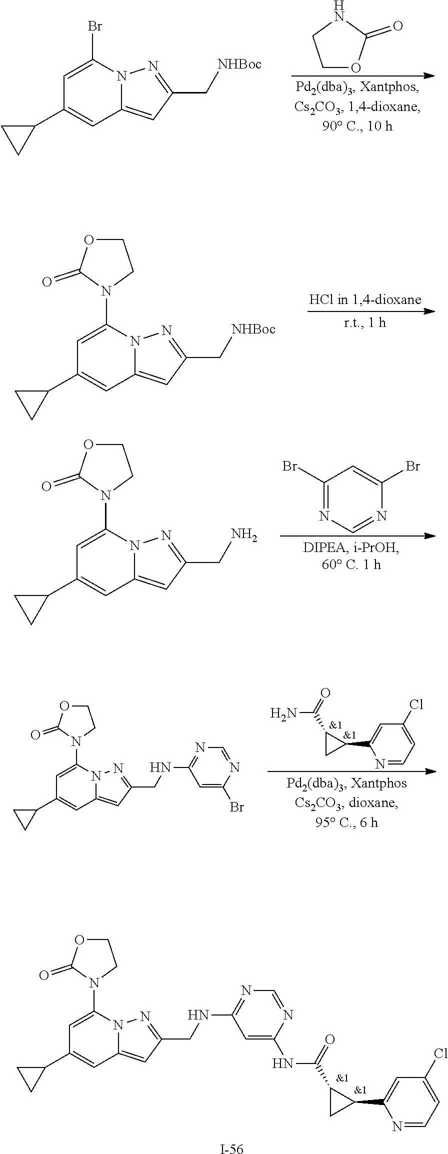

Synthesis of tert-butyl ((5-cyclopropyl-7-(2-oxooxazolidin-3-yl)pyrazolo[1,5-a]pyridin-2-yl)methyl)carbamate. To a mixture of tert-butyl ((7-bromo-5-cyclopropylpyrazolo[1,5-a]pyridin-2-yl)methyl)carbamate (300 mg, 0.82 mmol), oxazolidin-2-one (86 mg, 0.99 mmol) and Cs₂CO₃ (802 mg, 2.46 mmol) in 1,4-dioxane (8 mL) was added Pd₂(dba)₃ (75 mg, 0.082 mmol) and Xantphos (92 mg, 0.16 mmol). The mixture was stirred at 90° C. for 10 h under N₂. The reaction mixture was cooled to room temperature. The reaction mixture was filtered through Celite® and the filter cake was washed with DCM/MeOH (10/1, 30 mL). The filtrate was concentrated in vacuo to give the crude, which was purified by column chromatography (eluent: PE/EtOAc=1/1) to give tert-butyl ((5-cyclopropyl-7-(2-oxooxazolidin-3-yl)pyrazolo[1,5-a]pyridin-2-yl)methyl)carbamate (200 mg, 66%) as yellow oil. ESI-MS [M+H]⁺: 373.2.

Synthesis of 3-(2-(aminomethyl)-5-cyclopropylpyrazolo[1,5-a]pyridin-7-yl)oxazolidin-2-one. A solution of tert-butyl ((5-cyclopropyl-7-(2-oxooxazolidin-3-yl)pyrazolo[1,5-a]pyridin-2-yl)methyl)carbamate (200 mg, 0.54 mmol) in HCl (4M solution in 1,4-dioxane, 5 mL) was stirred at room temperature for 1 h. The reaction mixture was quenched with NaHCO₃ (sat. aq., 30 mL) and extracted with DCM/MeOH (10/1, 20 mL×3). The combined organic layers were washed with brine (50 mL), dried over anhydrous Na₂SO₄, and concentrated in vacuo to give the crude, which was purified by column chromatography (eluent: DCM/MeOH=10/1) to give 3-(2-(aminomethyl)-5-cyclopropylpyrazolo[1,5-a]pyridin-7-yl)oxazolidin-2-one (120 mg, 82%) as a yellow solid. ESI-MS [M+H]⁺: 273.2.

Synthesis of 3-(2-(((6-bromopyrimidin-4-yl)amino)methyl)-5-cyclopropylpyrazolo[1,5-a]pyridin-7-yl)oxazolidin-2-one. A mixture of 3-(2-(aminomethyl)-5-cyclopropylpyrazolo[1,5-a]pyridin-7-yl)oxazolidin-2-one (120 mg, 0.44 mmol), 4,6-dibromopyrimidine (157 mg, 0.66 mmol) and DIPEA (170 mg, 1.32 mmol) in i-PrOH (10 mL) was stirred at 60° C. for 1 h. The reaction mixture was concentrated in vacuo to give the crude, which was purified by preparative TLC (eluent: DCM/MeOH=10/1) to give 3-(2-(((6-bromopyrimidin-4-yl)amino)methyl)-5-cyclopropylpyrazolo[1,5-a]pyridin-7-yl)oxazolidin-2-one (150 mg, 80%) as a white solid. ESI-MS [M+H]⁺: 429.2.

Synthesis of rac-(1S*,2S*)-2-(4-chloropyridin-2yl)-N-(6-(((5-cyclopropyl-7-(2-oxooxazolidin-3-yl)pyrazolo[1,5-a]pyridin-2-yl)methyl)amino)pyrimidin-4-yl)cyclopropane-1-carboxamide. To a mixture of 3-(2-(((6-bromopyrimidin-4-yl)amino)methyl)-5-cyclopropylpyrazolo[1,5-a]pyridin-7-yl)oxazolidin-2-one (50 mg, 0.12 mmol), rac-(1S*,2S*)-2-(4-chloropyridin-2-yl)cyclopropane-1-carboxamide (24 mg, 0.12 mmol) and Cs₂CO₃ (117 mg, 0.36 mmol) in 1,4-dioxane (5 mL) was added Pd₂(dba)₃ (11 mg, 0.012 mmol) and Xantphos (7 mg, 0.012 mmol). The reaction mixture was stirred at 95° C. for 6 h under N₂. The reaction mixture was cooled to room temperature. The mixture was filtered through Celite® and the filter cake was washed with DCM/MeOH (10/1, 30 mL). The filtrate was concentrated to give the crude, which was purified by preparative TLC (DCM/MeOH=20/1) to give rac-(1S*,2S*)-2-(4-chloropyridin-2-yl)-N-(6-(((5-cyclopropyl-7-(2-oxooxazolidin-3-yl)pyrazolo[1,5-a]pyridin-2-yl)methyl)amino)pyrimidin-4-yl)cyclopropane-1-carboxamide (25 mg, 38%) as a white solid. ESI-MS [M+H]⁺: 545.2. ¹H NMR (400 MHz, DMSO) δ 10.65 (s, 1H), 8.42 (d, J=5.4 Hz, 1H), 8.19 (s, 1H), 7.93 (s, 1H), 7.65 (d, J=1.8 Hz, 1H), 7.36-7.34 (m, 2H), 7.30 (s, 1H), 6.73 (d, J=1.8 Hz, 1H), 6.40 (s, 1H), 4.64-4.57 (m, 4H), 4.22-4.18 (m, 2H), 2.60-2.56 (m, 2H), 2.03-1.96 (m, 1H), 1.58-1.42 (m, 2H), 1.04-0.97 (m, 2H), 0.80-0.73 (m, 2H).

### Example 57

### Synthesis of rac-(1S*,2S*)-2-(4-chloropyridin-2-yl)-N-(6-(((5-cyclopropyl-7-(3-methyl-2,4-dioxoimidazolidin-1-yl)pyrazolo[1,5-a]pyridin-2-yl)methyl)amino)pyrimidin-4-yl)cyclopropane-1-carboxamide (I-57)

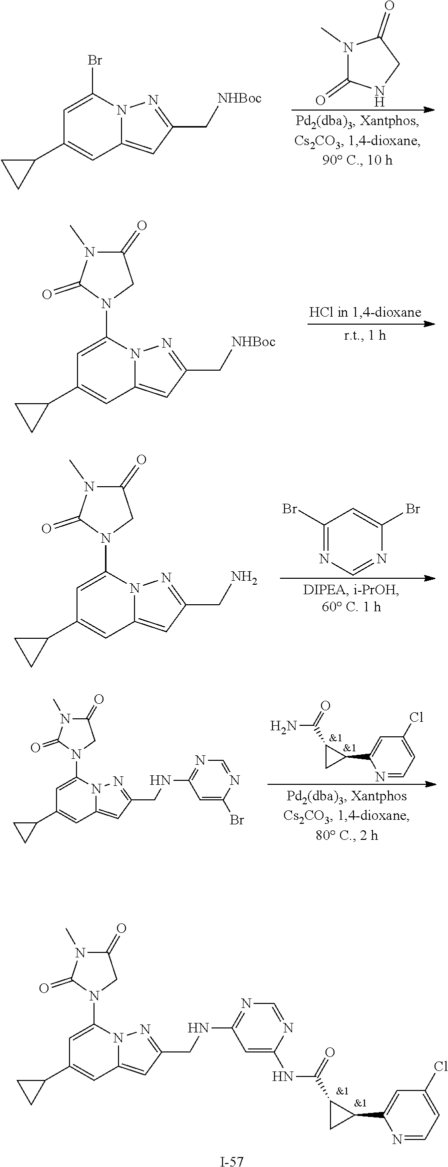

Synthesis of tert-butyl ((5-cyclopropyl-7-(3-methyl-2,4-dioxoimidazolidin-1-yl)pyrazolo[1,5-a]pyridin-2-yl)methyl)carbamate. To a mixture of tert-butyl ((7-bromo-5-cyclopropylpyrazolo[1,5-a]pyridin-2-yl)methyl)carbamate (300 mg, 0.82 mmol), 3-methylimidazolidine-2,4-dione (113 mg, 0.99 mmol) and Cs₂CO₃ (802 mg, 2.46 mmol) in 1,4-dioxane (8 mL) was added Pd₂(dba)₃ (75 mg, 0.082 mmol), Xantphos (92 mg, 0.16 mmol). The mixture was stirred at 90° C. for 10 h under N₂. The reaction mixture was cooled to room temperature. The reaction mixture was filtered through Celite® and the filter cake was washed with DCM/MeOH (10/1, 100 mL). The filtrate was concentrated in vacuo to give the crude, which was purified by preparative TLC (eluent: PE/EtOAc=1/1) to give tert-butyl ((5-cyclopropyl-7-(3-methyl-2,4-dioxoimidazolidin-1-yl)pyrazolo[1,5-a]pyridin-2-yl)methyl)carbamate (200 mg, 61%) as yellow oil. ESI-MS [M+H]⁺: 400.2.

Synthesis of 1-(2-(aminomethyl)-5-cyclopropylpyrazolo[1,5-a]pyridin-7-yl)-3-methylimidazolidine-2,4-dione. A mixture of tert-butyl ((5-cyclopropyl-7-(3-methyl-2,4-dioxoimidazolidin-1-yl)pyrazolo[1,5-a]pyridin-2-yl)methyl)carbamate (200 mg, 0.5 mmol) in HCl (4 M solution in 1,4-dioxane, 5 mL) was stirred at room temperature for 1 h. The reaction mixture was quenched with NaHCO₃ (sat. aq., 20 mL) and extracted with DCM/MeOH (10/1, 20 mL×3). The combined organic layers were washed with brine (50 mL), dried over anhydrous Na₂SO₄, and concentrated in vacuo to give the crude, which was purified by column chromatography (eluent: DCM/MeOH=10/1) to give 1-(2-(aminomethyl)-5-cyclopropylpyrazolo[1,5-a]pyridin-7-yl)-3-methylimidazolidine-2,4-dione (140 mg, 94%) as a yellow solid. ESI-MS [M+H]⁺: 300.2.

Synthesis of 1-(2-(((6-bromopyrimidin-4-yl)amino)methyl)-5-cyclopropylpyrazolo[1,5-a]pyridin-7-yl)-3-methylimidazolidine-2,4-dione. A mixture of 1-(2-(aminomethyl)-5-cyclopropylpyrazolo[1,5-a]pyridin-7-yl)-3-methylimidazolidine-2,4-dione (140 mg, 0.47 mmol), 4,6-dibromopyrimidine (168 mg, 0.71 mmol), and DIPEA (181 mg, 1.4 mmol) in i-PrOH (10 mL) was stirred at 60° C. for 1 h. The reaction mixture was concentrated in vacuo to give the crude, which was purified by preparative TLC (eluent: DCM/MeOH=10/1) to give 1-(2-(((6-bromopyrimidin-4-yl)amino)methyl)-5-cyclopropylpyrazolo[1,5-a]pyridin-7-yl)-3-methylimidazolidine-2,4-dione (180 mg, 84%) as a white solid. ESI-MS [M+H]⁺: 456.2.

Synthesis of rac-(1S*,2S*)-2-(4-chloropyridin-2-yl)-N-(6-(((5-cyclopropyl-7-(3-methyl-2,4-dioxoimidazolidin-1-yl)pyrazolo[1,5-a]pyridin-2-yl)methyl)amino)pyrimidin-4-yl)cyclopropane-1-carboxamide. To a mixture of 1-(2-(((6-bromopyrimidin-4-yl)amino)methyl)-5-cyclopropylpyrazolo[1,5-a]pyridin-7-yl)-3-methylimidazolidine-2,4-dione (50 mg, 0.11 mmol), rac-(1S*,2S*)-2-(4-chloropyridin-2-yl)cyclopropane-1-carboxamide (22 mg, 0.11 mmol) and Cs₂CO₃ (108 mg, 0.33 mmol) in 1,4-dioxane (6 mL) was added Pd₂(dba)₃ (10 mg, 0.011 mmol) and Xantphos (13 mg, 0.022 mmol). The reaction mixture was stirred at 80° C. for 2 h under N₂. The reaction mixture was cooled to room temperature. The mixture was filtered through Celite® and the filter cake was washed with DCM/MeOH (10/1, 30 mL). The filtrate was concentrated in vacuo to give the crude, which was purified by preparative TLC (eluent: DCM/MeOH=10/1) to give rac-(1S*,2S*)-2-(4-chloropyridin-2-yl)-N-(6-(((5-cyclopropyl-7-(3-methyl-2,4-dioxoimidazolidin-1-yl)pyrazolo[1,5-a]pyridin-2-yl)methyl)amino)pyrimidin-4-yl)cyclopropane-1-carboxamide (16 mg, 25%) as a white solid. ESI-MS [M+H]⁺: 572.2. ¹H NMR (400 MHz, DMSO) δ 10.65 (s, 1H), 8.42 (d, J=5.3 Hz, 1H), 8.18 (s, 1H), 7.91 (s, 1H), 7.64 (s, 1H), 7.37-7.33 (m, 2H), 7.30 (s, 1H), 6.75 (d, J=1.6 Hz, 1H), 6.42 (s, 1H), 4.67-4.61 (m, 4H), 3.01 (s, 3H), 2.65-2.55 (m, 2H), 2.03-1.97 (m, 1H), 1.54-1.46 (m, 2H), 1.05-0.97 (m, 2H), 0.78-0.71 (m, 2H).

### Example 58

### Synthesis of (1S,2S)-2-(3-chlorophenyl)-N-(6-(((6-cyclopropyl-8-(4-methylpiperazin-1-yl)-[1,2,4]triazolo[1,5-a]pyridin-2-yl)methyl)amino)pyrimidin-4-yl)cyclopropane-1-carboxamide (I-58)

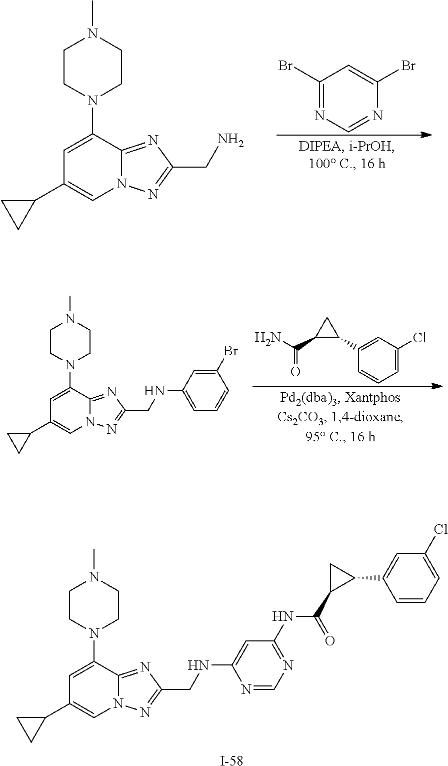

Synthesis of 6-bromo-N-((6-cyclopropyl-8-(4-methylpiperazin-1-yl)-[1,2,4]triazolo[1,5-a]pyridin-2-yl)methyl)pyrimidin-4-amine. A mixture of (6-cyclopropyl-8-(4-methylpiperazin-1-yl)-[1,2,4]triazolo[1,5-a]pyridin-2-yl)methanamine (45 mg, 0.16 mmol), 4,6-dibromopyrimidine (57 mg, 0.24 mmol) and DIPEA (103 mg, 0.8 mmol) in i-PrOH (10 mL) was stirred at 100° C. for 16 h. The reaction mixture was concentrated in vacuo to give the crude product, which was purified by preparative TLC (eluent: DCM/MeOH=10/1) to give 6-bromo-N-((6-cyclopropyl-8-(4-methylpiperazin-1-yl)-[1,2,4]triazolo[1,5-a]pyridin-2-yl)methyl)pyrimidin-4-amine (15 mg, 21%) as a white solid. ESI-MS [M+H]⁺: 443.2.

Synthesis of (1S,2S)-2-(3-chlorophenyl)-N-(6-(((6-cyclopropyl-8-(4-methylpiperazin-1-yl)-[1,2,4]triazolo[1,5-a]pyridin-2-yl)methyl)amino)pyrimidin-4-yl)cyclopropane-1-carboxamide. To a mixture of 6-bromo-N-((6-cyclopropyl-8-(4-methylpiperazin-1-yl)-[1,2,4]triazolo[1,5-a]pyridin-2-yl)methyl)pyrimidin-4-amine (15 mg, 0.034 mmol), (1S,2S)-2-(3-chlorophenyl)cyclopropane-1-carboxamide (10 mg, 0.05 mmol) and Cs₂CO₃ (33 mg, 0.10 mmol) in 1,4-dioxane (5 mL) was added Xantphos (8 mg, 0.014 mmol) and Pd₂(dba)₃ (6 mg, 0.007 mmol). The reaction mixture was cooled to room temperature. The mixture was filtered through Celite® and the filter cake was washed with DCM/MeOH (10/1, 30 mL). The filtrate was concentrated in vacuo to give the crude, which was purified by preparative TLC (eluent: DCM/MeOH=10/1) to give (1S,2S)-2-(3-chlorophenyl)-N-(6-(((6-cyclopropyl-8-(4-methylpiperazin-1-yl)-[1,2,4]triazolo[1,5-a]pyridin-2-yl)methyl)amino)pyrimidin-4-yl)cyclopropane-1-carboxamide (8 mg, 42%) as a white solid. ESI-MS [M+H]⁺: 558.2. ¹H NMR (400 MHz, DMSO) δ 10.29 (s, 1H), 8.18 (s, 1H), 7.74 (d, J=5.8 Hz, 1H), 7.43 (s, 1H), 7.35-7.18 (m, 4H), 7.11 (d, J=7.7 Hz, 1H), 6.97 (s, 1H), 4.44 (d, J=5.9 Hz, 2H), 3.50 (s, 4H), 2.51-2.49 (m, 4H), 2.44-2.31 (m, 2H), 2.24 (s, 3H), 2.00-1.92 (m, 1H), 1.51-1.39 (m, 1H), 1.38-1.31 (m, 1H), 0.97-0.86 (m, 2H), 0.78-0.75 (m, 2H).

### Example 59

### Synthesis of (1S,2S)—N-(6-(((6-chloro-8-(4-methylpiperazin-1-yl)quinolin-3-yl)methyl)amino)pyrimidin-4-yl)-2-(3-chlorophenyl)cyclopropane-1-carboxamide (I-59)

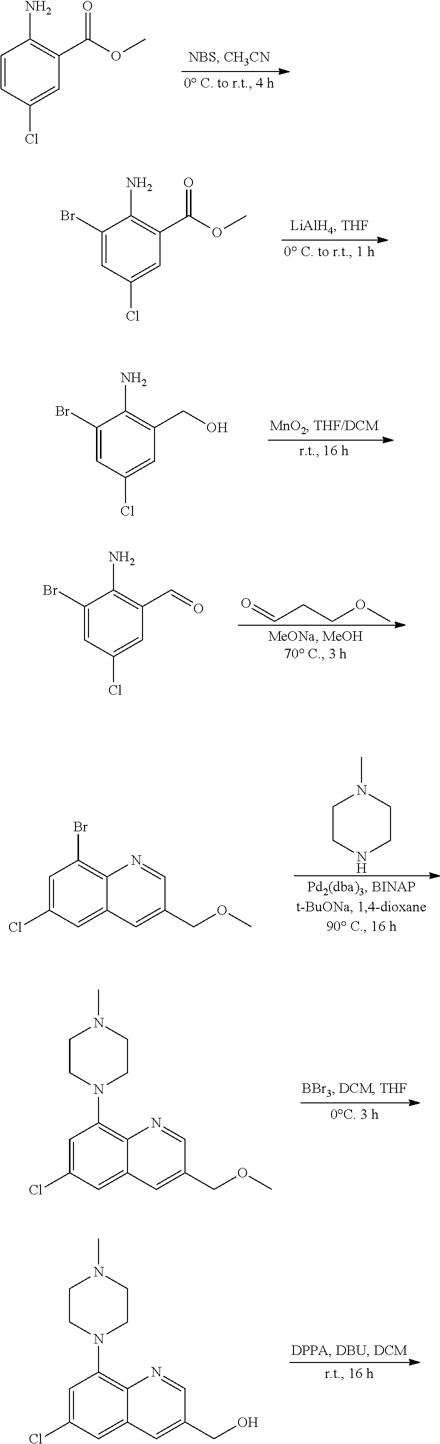

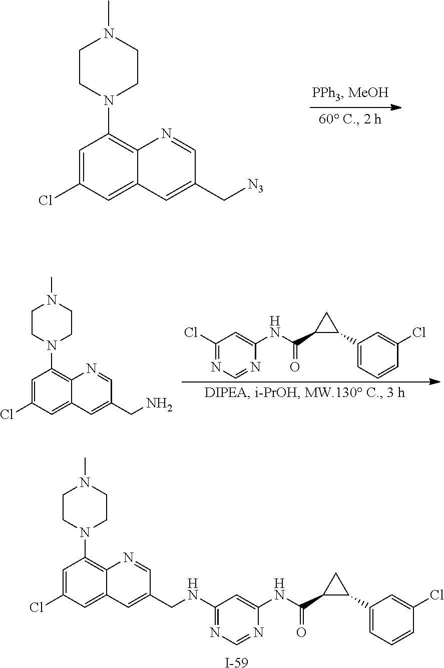

Synthesis of methyl 2-amino-3-bromo-5-chlorobenzoate. To a mixture of methyl 2-amino-5-chlorobenzoate (5 g, 27 mmol) in CH₃CN (50 mL) was added NBS (5.3 g, 30 mmol) at 0° C. The mixture was stirred at room temperature for 4 h. The reaction was quenched with water (100 mL) and extracted with EtOAc (50 mL×3). The combined organic layers were washed with brine (100 mL), dried over anhydrous Na₂SO₄, and concentrated in vacuo to give the crude, which was purified by column chromatography (eluent: DCM/MeOH=40/1) to give methyl 2-amino-3-bromo-5-chlorobenzoate (5 g, 70%) as a yellow solid. ESI-MS [M+H]⁺: 264.1.

Synthesis of (2-amino-3-bromo-5-chlorophenyl)methanol. To a mixture of methyl 2-amino-3-bromo-5-chlorobenzoate (5 g, 19 mmol) in THF (50 mL) was added LiAlH₄ (1M solution in THF, 57 mL, 57 mmol) at 0° C. The mixture was stirred at room temperature for 1 h. The reaction was cooled to 0° C., quenched with water (100 mL) and extracted with EtOAc (50 mL×3). The combined organic layers were washed with brine (100 mL), dried over anhydrous Na₂SO₄, and concentrated in vacuo to give the crude, which was purified by silica column chromatography eluting with a gradient of 0-5% MeOH in DCM to give (2-amino-3-bromo-5-chlorophenyl)methanol (4.0 g, 90%) as a yellow solid. ESI-MS [M+H]⁺: 236.1

Synthesis of 2-amino-3-bromo-5-chlorobenzaldehyde. To a mixture of (2-amino-3-bromo-5-chlorophenyl)methanol (4.0 g, 17 mmol) in THF (30 mL) and DCM (30 mL) was added MnO₂ (14.8 g, 170 mmol). The mixture was stirred at room temperature for 16 h. The reaction mixture was filtered through Celite® and the filter cake was washed with DCM/MeOH (10/1, 100 mL). The filtrate was concentrated in vacuo to give the crude, which was purified by silica column chromatography eluting with a gradient of 0-50% EtOAc in PE to give 2-amino-3-bromo-5-chlorobenzaldehyde (3.0 g, 76%) as a yellow solid. ESI-MS [M+H]⁺: 234.1.

Synthesis of 8-bromo-6-chloro-3-(methoxymethyl)quinolone. To a mixture of 3-methoxypropanal (572 mg, 6.5 mmol) and 2-amino-3-bromo-5-chlorobenzaldehyde (1.0 g, 4.3 mmol) in MeOH (20 mL) was added MeONa (5.4 M solution in MeOH, 1.2 mL, 6.5 mmol) at room temperature. The mixture was stirred at 70° C. for 3 h. After cooled to room temperature, the reaction was quenched with NH₄Cl (sat. aq., 40 mL) and extracted with EtOAc (30 mL×3). The combined organic layers were washed with brine (50 mL), dried over anhydrous Na₂SO₄ and concentrated in vacuo to give the crude, which was purified by silica column chromatography eluting with a gradient of 0-25% EtOAc in PE to give 8-bromo-6-chloro-3-(methoxymethyl)quinoline (900 mg, 73%) as a yellow solid. ESI-MS [M+H]⁺: 286.1.

Synthesis of 6-chloro-3-(methoxymethyl)-8-(4-methylpiperazin-1-yl)quinolone. To a mixture of 8-bromo-6-chloro-3-(methoxymethyl)quinoline (800 mg, 2.8 mmol), 1-methylpiperazine (840 mg, 8.4 mmol) and t-BuONa (806 mg, 8.4 mmol) in 1,4-dioxane (15 mL) was added BINAP (349 mg, 0.56 mmol) and Pd₂(dba)₃ (256 mg, 0.28 mmol). The mixture was stirred at 90° C. for 16 h under N₂. The reaction mixture was cooled to room temperature. The mixture was filtered through Celite® and the filter cake was washed with DCM/MeOH (10/1, 50 mL). The filtrate was concentrated in vacuo to give the crude, which was purified by silica column chromatography eluting with a gradient of 0-5% MeOH in DCM to give 6-chloro-3-(methoxymethyl)-8-(4-methylpiperazin-1-yl)quinoline (400 mg, 47%) as a brown oil. ESI-MS [M+H]⁺: 306.1.

Synthesis of (6-chloro-8-(4-methylpiperazin-1-yl)quinolin-3-yl)methano. To a mixture of 6-chloro-3-(methoxymethyl)-8-(4-methylpiperazin-1-yl)quinoline (400 mg, 1.3 mmol) in DCM (10 mL) was added BBr₃ (975 mg, 3.9 mmol) dropwise at 0° C. The reaction mixture was stirred at 0° C. for 3 h. The reaction was quenched with water (50 mL) and extracted with DCM/MeOH (10/1, 30 mL×3). The combined organic was dried over anhydrous Na₂SO₄ and concentrated in vacuo to give the crude product, which was purified by preparative TLC (eluent: DCM/MeOH=5/1) to give (6-chloro-8-(4-methylpiperazin-1-yl)quinolin-3-yl)methanol (70 mg, 19%) as a yellow oil. ESI-MS [M+H]⁺: 292.2.

Synthesis of 3-(azidomethyl)-6-chloro-8-(4-methylpiperazin-1-yl)quinolone. To a mixture of (6-chloro-8-(4-methylpiperazin-1-yl)quinolin-3-yl)methanol (70 mg, 0.24 mmol) and DBU (182 mg, 1.2 mmol) in DCM (5 mL) was added DPPA (330 mg, 1.2 mmol) dropwise at 0° C. The mixture was stirred at room temperature for 16 h. The reaction was quenched with water (30 mL) and extracted with DCM (20 mL×3). The combined organic layers were washed with brine (30 mL), dried over anhydrous Na₂SO₄, and concentrated in vacuo to give the crude product, which was purified by preparative TLC (eluent: DCM/MeOH=10/1) to give 3-(azidomethyl)-6-chloro-8-(4-methylpiperazin-1-yl)quinoline (70 mg, 92%) as a yellow oil. ESI-MS [M+H]⁺: 317.2.

Synthesis of (6-chloro-8-(4-methylpiperazin-1-yl)quinolin-3-yl)methanamine. A mixture of 3-(azidomethyl)-6-chloro-8-(4-methylpiperazin-1-yl)quinoline (70 mg, 0.22 mmol) and PPh₃ (87 mg, 0.33 mmol) in MeOH (5 mL) was stirred at 60° C. for 2 h. The mixture was concentrated in vacuo to give the crude product, which was purified by preparative TLC (eluent: DCM/MeOH=5/1) to give (6-chloro-8-(4-methylpiperazin-1-yl)quinolin-3-yl)methanamine (40 mg, 63%) as a yellow oil. ESI-MS [M+H]⁺: 291.2.

Synthesis of (1S,2S)—N-(6-(((6-chloro-8-(4-methylpiperazin-1-yl)quinolin-3-yl)methyl)amino)pyrimidin-4-yl)-2-(3-chlorophenyl)cyclopropane-1-carboxamide. A mixture of (6-chloro-8-(4-methylpiperazin-1-yl)quinolin-3-yl)methanamine (40 mg, 0.14 mmol), (1S,2S)-2-(3-chlorophenyl)-N-(6-chloropyrimidin-4-yl)cyclopropane-1-carboxamide (43 mg, 0.14 mmol) and DIPEA (54 mg, 0.42 mmol) in i-PrOH (2 mL) was stirred in a sealed tube. The reaction was irradiated in a microwave reactor at 130° C. for 3 h. The reaction was concentrated in vacuo to give the crude product, which was purified by preparative TLC (eluent: DCM/MeOH=10/1) to give (1S,2S)—N-(6-(((6-chloro-8-(4-methylpiperazin-1-yl)quinolin-3-yl)methyl)amino)pyrimidin-4-yl)-2-(3-chlorophenyl)cyclopropane-1-carboxamide (8.5 mg, 11%) as a yellow solid. ESI-MS [M+H]⁺: 562.2. ¹H NMR (400 MHz, DMSO) δ 10.65 (s, 1H), 8.80 (s, 1H), 8.20 (s, 1H), 8.08 (s, 2H), 7.58 (s, 1H), 7.32-7.25 (m, 4H), 7.16-7.14 (m, 1H), 6.98 (s, 1H), 4.69 (s, 2H), 3.45 (s, 4H), 2.56 (s, 4H), 2.40-2.38 (m, 2H), 2.26 (s, 3H), 1.49-1.38 (m, 2H).

### Example 60

### Synthesis of (1S,2S)-2-(3-chlorophenyl)-N-(6-((2R,4S)-2-(6-cyclopropylimidazo[1,2-b]pyridazin-2-yl)-4-hydroxypyrrolidin-1-yl)pyrimidin-4-yl)cyclopropane-1-carboxamide (I-60)

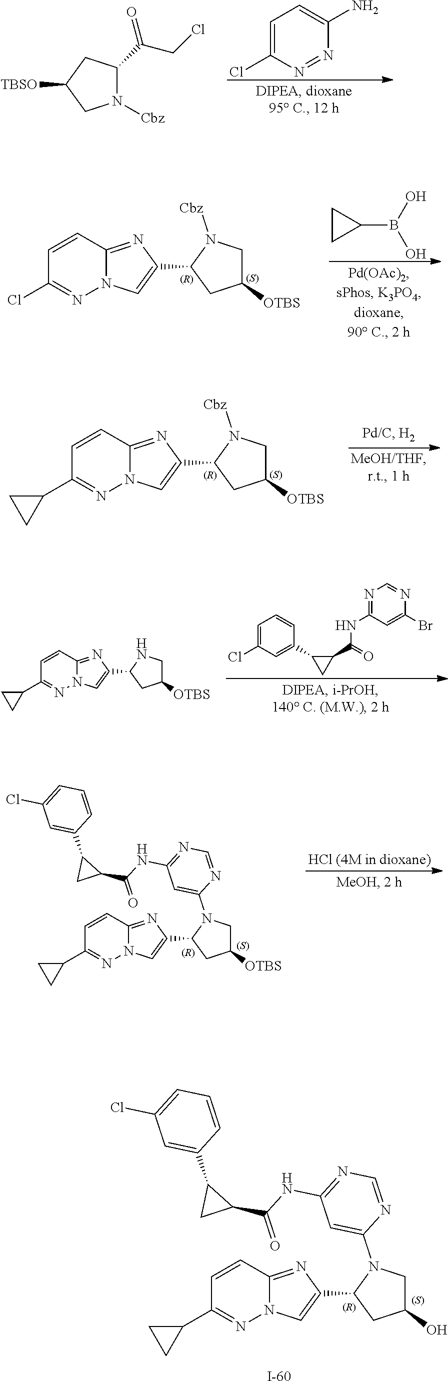

Synthesis of benzyl (2R,4S)-4-((tert-butyldimethylsilyl)oxy)-2-(6-chloroimidazo[1,2-b]pyridazin-2-yl)pyrrolidine-1-carboxylate. To a mixture of benzyl (2R,4S)-4-((tert-butyldimethylsilyl)oxy)-2-(2-chloroacetyl)pyrrolidine-1-carboxylate (2 g, 4.86 mmol) in dioxane (15 mL) was added 6-chloropyridazin-3-amine (627 mg, 4.86 mmol) and DIPEA (3.14 g, 24.3 mmol) under N₂. The reaction mixture was stirred at 95° C. for 12 h. After cooling to room temperature, diluted with water (50 mL) and extracted with EtOAc (30 mL×3). The combined organic layers were washed with brine (50 mL), dried over anhydrous Na₂SO₄, and concentrated in vacuo to give the crude product, which was purified by silica gel chromatography (eluent: DCM/MeOH=40/1) to give benzyl (2R,4S)-4-((tert-butyldimethylsilyl)oxy)-2-(6-chloroimidazo[1,2-b]pyridazin-2-yl)pyrrolidine-1-carboxylate (720 mg, 31%) as a yellow solid. ESI-MS [M+H]⁺: 487.2.

Synthesis of benzyl (2R,4S)-4-((tert-butyldimethylsilyl)oxy)-2-(6-cyclopropylimidazo[1,2-b]pyridazin-2-yl)pyrrolidine-1-carboxylate. To a mixture of benzyl (2R,4S)-4-((tert-butyldimethylsilyl)oxy)-2-(6-chloroimidazo[1,2-b]pyridazin-2-yl)pyrrolidine-1-carboxylate (720 mg, 1.48 mmol), cyclopropylboronic acid (134 mg, 1.56 mmol), and K₃PO₄ (940 mg, 4.44 mmol) in dioxane (25 mL) was added Pd₂(OAc)₂ (34 mg, 0.15 mmol) and SPhos (123 mg, 0.30 mmol). The reaction mixture was stirred at 90° C. for 2 h under N₂. The reaction mixture was cooled to room temperature, filtered through Celite® and the filter cake was washed with DCM/MeOH (10/1, 50 mL). The filtrate was concentrated in vacuo to give the crude, which was purified by column chromatography (eluent: DCM/MeOH=50/1˜ 20/1) to give benzyl (2R,4S)-4-((tert-butyldimethylsilyl)oxy)-2-(6-cyclopropylimidazo[1,2-b]pyridazin-2-yl)pyrrolidine-1-carboxylate (400 mg, 55%) as a yellow solid. ESI-MS [M+H]⁺: 493.3.

Synthesis of 2-((2R,4S)-4-((tert-butyldimethylsilyl)oxy)pyrrolidin-2-yl)-6-cyclopropylimidazo[1,2-b]pyridazine. A mixture of benzyl (2R,4S)-4-((tert-butyldimethylsilyl)oxy)-2-(6-cyclopropylimidazo[1,2-b]pyridazin-2-yl)pyrrolidine-1-carboxylate (160 mg, 0.33 mmol) and Pd/C (30 mg) in MeOH (6 mL) and THF (6 mL) was stirred at room temperature for 1 h under and atmosphere of H₂. The reaction mixture was filtered through Celite® and the filter cake was washed with DCM/MeOH (10/1, 50 mL). The filtrate was concentrated in vacuo in vacuo to give 2-((2R,4S)-4-((tert-butyldimethylsilyl)oxy)pyrrolidin-2-yl)-6-cyclopropylimidazo[1,2-b]pyridazine (110 mg, crude) as a yellow solid. ESI-MS [M+H]⁺: 359.2.

Synthesis of (1S,2S)—N-(6-((2R,4S)-4-((tert-butyldimethylsilyl)oxy)-2-(6-cyclopropylimidazo[1,2-b]pyridazin-2-yl)pyrrolidin-1-yl)pyrimidin-4-yl)-2-(3-chlorophenyl)cyclopropane-1-carboxamide. A mixture of 2-((2R,4S)-4-((tert-butyldimethylsilyl)oxy)pyrrolidin-2-yl)-6-cyclopropylimidazo[1,2-b]pyridazine (110 mg, crude), (1S,2S)—N-(6-bromopyrimidin-4-yl)-2-(3-chlorophenyl)cyclopropane-1-carboxamide (108 mg, 0.31 mol), DIPEA (198 mg, 1.53 mmol) in i-PrOH (10 mL) was stirred in a sealed tube. After degassing with N₂ for 1 min, the reaction was irradiated in a microwave reactor at 140° C. for 2 h. After cooling to room temperature, the reaction was quenched with NaHCO₃ (sat. aq., 30 mL) and extracted with DCM (30 mL×3). The combined organic layers were washed with brine (30 mL), dried over Na₂SO₄ and concentrated in vacuo to give the crude, which was purified by preparative TLC (eluent: DCM/MeOH=40/1) to give (1S,2S)—N-(6-((2R,4S)-4-((tert-butyldimethylsilyl)oxy)-2-(6-cyclopropylimidazo[1,2-b]pyridazin-2-yl)pyrrolidin-1-yl)pyrimidin-4-yl)-2-(3-chlorophenyl)cyclopropane-1-carboxamide (60 mg, 31%) as a white solid. ESI-MS [M+H]⁺: 630.3.

Synthesis of (1S,2S)-2-(3-chlorophenyl)-N-(6-((2R,4S)-2-(6-cyclopropylimidazo[1,2-b]pyridazin-2-yl)-4-hydroxypyrrolidin-1-yl)pyrimidin-4-yl)cyclopropane-1-carboxamide. To a solution of (1S,2S)—N-(6-((2R,4S)-4-((tert-butyldimethylsilyl)oxy)-2-(6-cyclopropylimidazo[1,2-b]pyridazin-2-yl)pyrrolidin-1-yl)pyrimidin-4-yl)-2-(3-chlorophenyl)cyclopropane-1-carboxamide (60 mg, 0.10 mmol) in MeOH (8 mL) was added HCl (4M solution in dioxane, 3 mL). The resulting mixture was stirred at room temperature for 2 h. Then the reaction was quenched with NaHCO₃ (sat. aq., 30 mL) and extracted with DCM (30 mL×3). The combined organic layers were washed with brine (30 mL), dried over Na₂SO₄ and concentrated in vacuo to give the crude, which was purified by preparative TLC (eluent: DCM/MeOH=20/1) to give (1S,2S)-2-(3-chlorophenyl)-N-(6-((2R,4S)-2-(6-cyclopropylimidazo[1,2-b]pyridazin-2-yl)-4-hydroxypyrrolidin-1-yl)pyrimidin-4-yl)cyclopropane-1-carboxamide (27 mg, 53%) as a white solid. ESI-MS [M+H]⁺: 516.2. ¹H NMR (400 MHz, cd3od) δ 8.15 (s, 1H), 7.79-7.71 (m, 1H), 7.28-7.22 (m, 2H), 7.18-7.03 (m, 5H), 5.19 (s, 1H), 4.91 (s, 1H), 4.61 (s, 1H), 3.98-3.96 (m, 1H), 2.43-2.40 (m, 4H), 2.12-2.10 (m, 1H), 1.28 (s, 2H), 1.08-1.03 (m, 4H).

### Example 61

### Synthesis of (1S,2S)-2-(4-chloropyridin-2-yl)-N-(6-((2R,4S)-2-(6-cyclopropylimidazo[1,2-b]pyridazin-2-yl)-4-hydroxypyrrolidin-1-yl)pyrimidin-4-yl)cyclopropane-1-carboxamide (I-61)

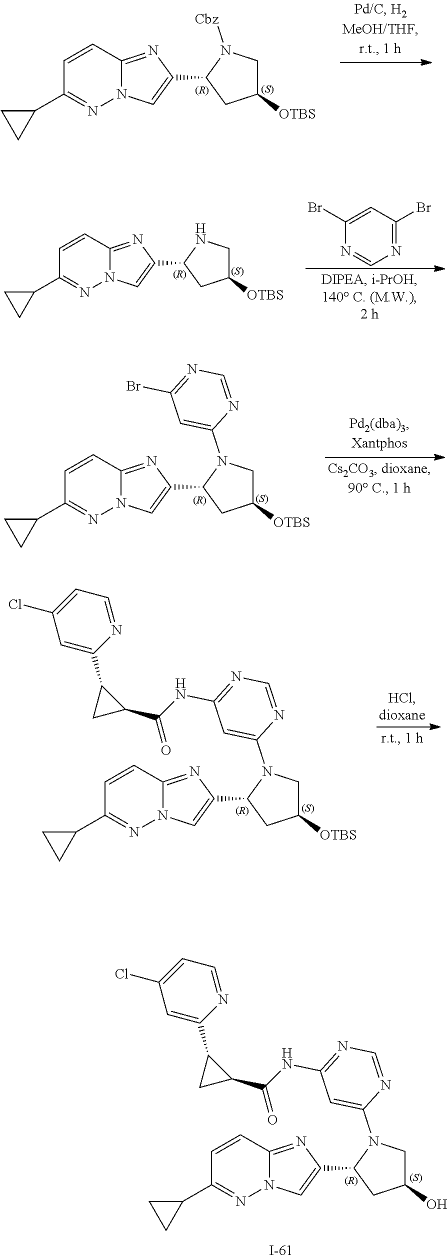

Synthesis of 2-((2R,4S)-4-((tert-butyldimethylsilyl)oxy)pyrrolidin-2-yl)-6-cyclopropylimidazo[1,2-b]pyridazine. To a solution of benzyl (2R,4S)-4-((tert-butyldimethyl silyl)oxy)-2-(6-cyclopropylimidazo[1,2-b]pyridazin-2-yl)pyrrolidine-1-carboxylate (200 mg, 0.41 mmol) in MeOH (6 mL) and THF (6 mL) was added Pd/C (20 mg). The mixture was stirred at room temperature for 1 h under H₂. The reaction mixture was filtered through Celite® and the filter cake was washed with DCM/MeOH (10/1, 50 mL). The filtrate was concentrated in vacuo to give the crude, which was purified by column chromatography (eluent: MeOH/DCM=0˜ 5%) to give 2-((2R,4S)-4-((tert-butyldimethylsilyl)oxy)pyrrolidin-2-yl)-6-cyclopropylimidazo[1,2-b]pyridazine (120 mg, 82%) as a white solid. ESI-MS [M+H]⁺: 359.2.

Synthesis of 2-((2R,4S)-1-(6-bromopyrimidin-4-yl)-4-((tert-butyldimethylsilyl)oxy) pyrrolidin-2-yl)-6-cyclopropylimidazo[1,2-b]pyridazine. A mixture of 2-((2R,4S)-4-((tert-butyldimethylsilyl)oxy)pyrrolidin-2-yl)-6-cyclopropylimidazo[1,2-b]pyridazine (120 mg, 0.34 mol), 4,6-dibromopyrimidine (80 mg, 0.34 mol) and DIPEA (219 mg, 1.70 mmol) in i-PrOH (10 mL) was stirred in a sealed tube. After degassing with N₂ for 1 min, the reaction was irradiated in microwave at 100° C. for 1 h. After cooling to room temperature, the reaction was quenched with NaHCO₃ (sat. aq., 30 mL) and extracted with DCM (30 mL×3). The combined organic layers were washed with brine (30 mL), dried over Na₂SO₄, and concentrated to give the crude, which was purified by preparative TLC (eluent: MeOH/DCM=1/20) to give 2-((2R,4S)-1-(6-bromopyrimidin-4-yl)-4-((tert-butyldimethylsilyl)oxy)pyrrolidin-2-yl)-6-cyclopropylimidazo[1,2-b]pyridazine (100 mg, 57%) as a white solid. ESI-MS [M+H]⁺: 517.2.

Synthesis of (1S,2S)—N-(6-((2R,4S)-4-((tert-butyldimethylsilyl)oxy)-2-(6-cyclo propylimidazo[1,2-b]pyridazin-2-yl)pyrrolidin-1-yl)pyrimidin-4-yl)-2-(4-chloropyridin-2-yl)cyclopropane-1-carboxamide. To a mixture of 2-((2R,4S)-1-(6-bromopyrimidin-4-yl)-4-((tert-butyldimethylsilyl)oxy)pyrrolidin-2-yl)-6-cyclopropylimidazo[1,2-b]pyridazine (100 mg, 0.19 mmol), (1S,2S)-2-(4-chloropyridin-2-yl)cyclopropane-1-carboxamide (37 mg, 0.20 mmol), Cs₂CO₃ (190 mg, 0.58 mmol) in 1,4-dioxane (25 mL) was added Pd₂(dba)₃ (18 mg, 0.02 mmol) and Xantphos (23 mg, 0.04 mmol). The mixture was stirred at 90° C. for 1 h under N₂. After cooling to room temperature, the reaction mixture was filtered through Celite® and the filter cake was washed with DCM/MeOH (10/1, 50 mL). The filtrate was concentrated in vacuo to give the crude, which was purified by silica gel chromatography (DCM/MeOH=20/1) to give (1S,2S)—N-(6-((2R,4S)-4-((tert-butyldimethylsilyl)oxy)-2-(6-cyclopropylimidazo[1,2-b]pyridazin-2-yl)pyrrolidin-1-yl)pyrimidin-4-yl)-2-(4-chloropyridin-2-yl)cyclopropane-1-carboxamide (55 mg, 46%) as a white solid. ESI-MS [M+H]⁺: 631.3.

Synthesis of (1S,2S)-2-(4-chloropyridin-2-yl)-N-(6-((2R,4S)-2-(6-cyclopropyl imidazo[1,2-b]pyridazin-2-yl)-4-hydroxypyrrolidin-1-yl)pyrimidin-4-yl)cyclopropane-1-carboxamide. To a solution of (1S,2S)—N-(6-((2R,4S)-4-((tert-butyldimethylsilyl)oxy)-2-(6-cyclopropylimidazo[1,2-b]pyridazin-2-yl)pyrrolidin-1-yl)pyrimidin-4-yl)-2-(4-chloropyridin-2-yl)cyclopropane-1-carboxamide (55 mg, 0.09 mmol) in MeOH (8 mL) was added HCl (4M solution in dioxane, 3 mL). The resulting mixture was stirred at room temperature for 1 h. The reaction was quenched with NaHCO₃ (sat. aq., 30 mL) and extracted with DCM (30 mL×3). The combined organic layers were washed with brine (30 mL), dried over Na₂SO₄ and concentrated in vacuo to give the crude, which was purified by preparative TLC (eluent: MeOH/DCM=1/10) to give (1S,2S)-2-(4-chloropyridin-2-yl)-N-(6-((2R,4S)-2-(6-cyclopropylimidazo[1,2-b]pyridazin-2-yl)-4-hydroxypyrrolidin-1-yl)pyrimidin-4-yl)cyclopropane-1-carboxamide (25 mg, 53%) as a white solid. ESI-MS [M+H]⁺: 517.2. ¹H NMR (400 MHz, cd3od) δ 8.33 (d, J=5.4 Hz, 1H), 8.14 (s, 1H), 7.87-7.79 (m, 1H), 7.73 (d, J=9.4 Hz, 1H), 7.43 (s, 1H), 7.25-7.23 (m, 2H), 7.05 (d, J=9.4 Hz, 1H), 5.18 (s, 1H), 4.91-4.86 (m, 1H), 4.62-4.61 (m, 1H), 3.99-3.95 (m, 1H), 2.63-2.55 (m, 1H), 2.50-2.38 (m, 3H), 2.15-2.08 (m, 1H), 1.56 (s, 2H), 1.10-0.98 (m, 4H).

### Example 62

### Synthesis of (S,2S)-2-(3-chlorophenyl)-N-(6-(((6-cyclopropyl-8-(3-methyl-2,4-dioxoimidazolidin-1-yl)imidazo[1,2-b]pyridazin-2-yl)methyl)amino)pyrimidin-4-yl)cyclopropane-1-carboxamide (I-62)

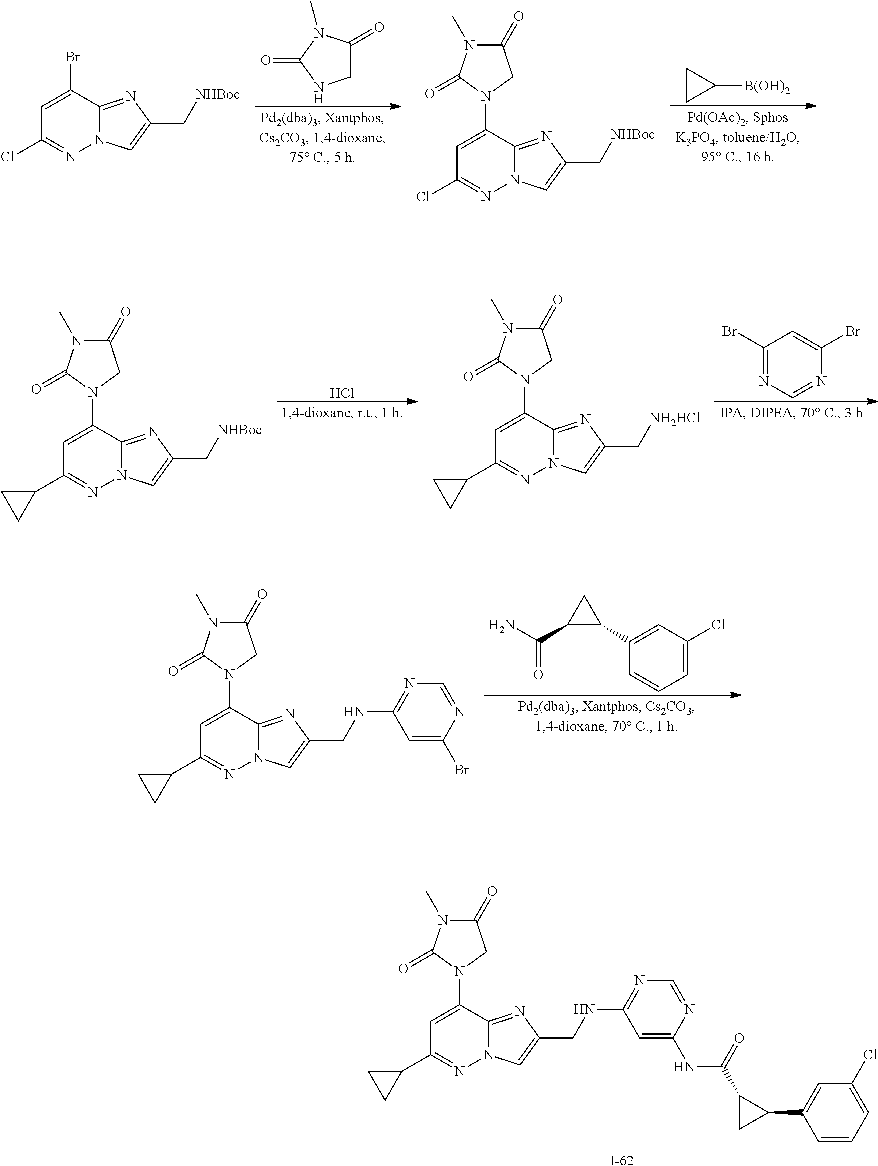

Synthesis of tert-butyl ((6-chloro-8-(3-methyl-2,4-dioxoimidazolidin-1-yl)imidazo[1,2-b]pyridazin-2-yl)methyl)carbamate. To a mixture of tert-butyl ((8-bromo-6-chloroimidazo[1,2-b]pyridazin-2-yl)methyl)carbamate (500 mg, 1.38 mmol), 3-methylimidazolidine-2,4-dione (157 mg, 1.38 mmol) and Cs₂CO₃ (1.35 g, 4.14 mmol) in 1,4-dioxane (10.0 mL) was added Pd₂(dba)₃ (128 mg, 0.14 mmol) and Xantphos (162 mg, 0.28 mmol). The reaction mixture was stirred at 75° C. for 5 h under N₂. The reaction mixture was filtered through Celite® and the filter cake was washed with DCM/MeOH (10/1, 50 mL). The filtrate was concentrated in vacuo to give the crude, which was purified by column chromatography (eluent: DCM/MeOH=20/1) to give tert-butyl ((6-chloro-8-(3-methyl-2,4-dioxoimidazolidin-1-yl)imidazo[1,2-b]pyridazin-2-yl)methyl)carbamate (300 mg, 55%) as a yellow solid. ESI-MS [M+H]⁺: 395.2.

Synthesis of tert-butyl ((6-cyclopropyl-8-(3-methyl-2,4-dioxoimidazolidin-1-yl)imidazo[1,2-b]pyridazin-2-yl)methyl)carbamate. A mixture of tert-butyl ((6-chloro-8-(3-methyl-2,4-dioxoimidazolidin-1-yl)imidazo[1,2-b]pyridazin-2-yl)methyl)carbamate (300 mg, 0.76 mmol), cyclopropylboronic acid (196 mg, 2.28 mmol), Pd(OAc)₂ (17.0 mg, 0.076 mmol), Sphos (62 mg, 0.15 mmol) and K₃PO₄ (483 mg, 2.28 mmol) in toluene/water (10.0 mL/1.0 mL) was stirred at 95° C. under N₂ for 16 h. After cooling to room temperature, H₂O (50 mL) was added and the mixture was extracted with EtOAc (30 mL×3). The combined organic layers were washed with brine (30 mL), dried over anhydrous Na₂SO₄, and concentrated in vacuo to give the crude, which was purified by column chromatography (eluent: DCM/MeOH=15/1) to give tert-butyl ((6-cyclopropyl-8-(3-methyl-2,4-dioxoimidazolidin-1-yl)imidazo[1,2-b]pyridazin-2-yl)methyl)carbamate (250 mg, 82%) as a yellow solid. ESI-MS [M+H]⁺: 401.2.

Synthesis of 1-(2-(aminomethyl)-6-cyclopropylimidazo[1,2-b]pyridazin-8-yl)-3-methylimidazolidine-2,4-dione hydrochloride. A mixture of tert-butyl ((6-cyclopropyl-8-(3-methyl-2,4-dioxoimidazolidin-1-yl)imidazo[1,2-b]pyridazin-2-yl)methyl)carbamate (200 mg, 0.5 mmol) in HCl (5.0 mL, 4M in 1,4-dioxane) was stirred at room temperature for 1 h. The mixture was concentrated in vacuo to give the product 1-(2-(aminomethyl)-6-cyclopropylimidazo[1,2-b]pyridazin-8-yl)-3-methylimidazolidine-2,4-dione as the hydrochloric acid salt (160 mg, 95%) as a yellow solid. ESI-MS [M+H]⁺: 301.2.

Synthesis of 1-(2-(((6-bromopyrimidin-4-yl)amino)methyl)-6-cyclopropylimidazo[1,2-b]pyridazin-8-yl)-3-methylimidazolidine-2,4-dione. A mixture of 1-(2-(aminomethyl)-6-cyclopropylimidazo[1,2-b]pyridazin-8-yl)-3-methylimidazolidine-2,4-dione hydrochloride (160 mg, 0.48 mmol), 4,6-dibromopyrimidine (227 mg, 0.96 mmol) and DIPEA (310 mg, 2.4 mmol) in i-PrOH (10 mL) was stirred at 70° C. under N₂ for 3 h. The reaction was quenched with NaHCO₃ (sat. aq., 30 mL) and extracted with DCM (20 mL×3). The combined organic layers were washed with brine (30 mL), dried over Na₂SO₄ and concentrated in vacuo to give the crude, which was purified by preparative TLC (eluent: DCM/MeOH=20/1) to give 1-(2-(((6-bromopyrimidin-4-yl)amino)methyl)-6-cyclopropylimidazo[1,2-b]pyridazin-8-yl)-3-methylimidazolidine-2,4-dione (120 mg, 55%) as a yellow solid. ESI-MS [M+H]⁺: 457.2.

Synthesis of (1S,2S)-2-(3-chlorophenyl)-N-(6-(((6-cyclopropyl-8-(3-methyl-2,4-dioxoimidazolidin-1-yl)imidazo[1,2-b]pyridazin-2-yl)methyl)amino)pyrimidin-4-yl)cyclopropane-1-carboxamide. To a mixture of 1-(2-(((6-bromopyrimidin-4-yl)amino)methyl)-6-cyclopropylimidazo[1,2-b]pyridazin-8-yl)-3-methylimidazolidine-2,4-dione (46 mg, 0.1 mmol), (1S,2S)-2-(3-chlorophenyl)cyclopropane-1-carboxamide (19.5 mg, 0.1 mmol) and Cs₂CO₃ (98 mg, 0.3 mmol) in 1,4-dioxane (5.0 mL) was added Pd₂(dba)₃ (9 mg, 0.01 mmol) and Xantphos (12 mg, 0.02 mmol). The reaction mixture was stirred at 70° C. for 1 h under N₂. The reaction mixture was filtered through Celite® and the filter cake was washed with DCM/MeOH (10/1, 20 mL). The filtrate was concentrated in vacuo to give the crude, which was purified by preparative TLC (eluent: DCM/MeOH=15/1) to give (1S,2S)-2-(3-chlorophenyl)-N-(6-(((6-cyclopropyl-8-(3-methyl-2,4-dioxoimidazolidin-1-yl)imidazo[1,2-b]pyridazin-2-yl)methyl)amino)pyrimidin-4-yl)cyclopropane-1-carboxamide (15 mg, 26%) as a white solid. ESI-MS [M+H]⁺: 572.2. ¹H NMR (400 MHz, DMSO) δ 10.61 (s, 1H), 8.19 (s, 1H), 7.91 (s, 1H), 7.85 (s, 1H), 7.69 (s, 1H), 7.42-7.22 (m, 4H), 7.20-7.03 (m, 1H), 5.11 (s, 2H), 4.58 (s, 2H), 2.44-2.33 (m, 2H), 2.13-2.08 (m, 1H), 1.55-1.32 (m, 2H), 1.05-1.03 (m, 2H), 0.98-0.73 (m, 2H).

### Example 63

### Synthesis of (1S,2S)-2-(4-chloropyridin-2-yl)-N-(6-(((6-cyclopropyl-8-(3-methyl-2,4-dioxoimidazolidin-1-yl)imidazo[1,2-b]pyridazin-2-yl)methyl)amino)pyrimidin-4-yl)cyclopropane-1-carboxamide (I-63)

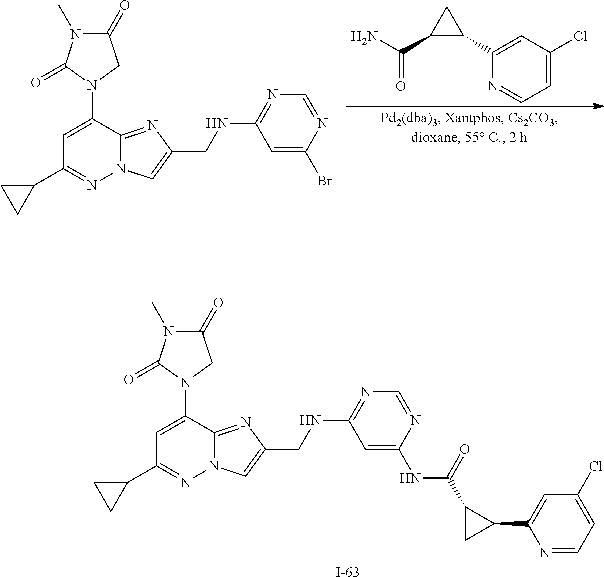

To a mixture of 1-(2-(((6-bromopyrimidin-4-yl)amino)methyl)-6-cyclopropylimidazo[1,2-b]pyridazin-8-yl)-3-methylimidazolidine-2,4-dione (46 mg, 0.1 mmol), (1S,2S)-2-(4-chloropyridin-2-yl)cyclopropane-1-carboxamide (20 mg, 0.1 mmol) and Cs₂CO₃ (98 mg, 0.3 mmol) in 1,4-dioxane (5 mL) was added Pd₂(dba)₃ (9.2 mg, 0.01 mmol) and Xantphos (11.6 mg, 0.02 mmol). The reaction mixture was stirred at 55° C. for 2 h under N₂. After cooling to room temperature, the reaction mixture was filtered through Celite® and the filter cake was washed with DCM/MeOH (10/1, 20 mL). The filtrate was concentrated in vacuo to give the crude, which was purified by preparative HPLC to give (1S,2S)-2-(4-chloropyridin-2-yl)-N-(6-(((6-cyclopropyl-8-(3-methyl-2,4-dioxoimidazolidin-1-yl)imidazo[1,2-b]pyridazin-2-yl)methyl)amino)pyrimidin-4-yl)cyclopropane-1-carboxamide (6 mg, 10%) as a white solid. ESI-MS [M+H]⁺: 573.2. ¹H NMR (400 MHz, DMSO) δ 10.66 (s, 1H), 8.44-8.40 (m, 2H), 8.19 (s, 1H), 7.91 (s, 1H), 7.87 (s, 1H), 7.70 (s, 1H), 7.64 (s, 1H), 7.36-7.34 (m, 2H), 5.11 (s, 2H), 4.58 (s, 2H), 2.98 (s, 3H), 2.67-2.54 (m, 2H), 2.15-2.10 (m, 1H), 1.62-1.43 (m, 2H), 1.07-1.03 (m, 2H), 0.96-0.80 (m, 2H).

### Example 64

### Synthesis of (2R,4S)-1-(6-((1S,2S)-2-(3-chlorophenyl)cyclopropane-1-carboxamido)pyrimidin-4-yl)-N-(4-cyclopropylphenyl)-4-hydroxypyrrolidine-2-carboxamide (I-64)

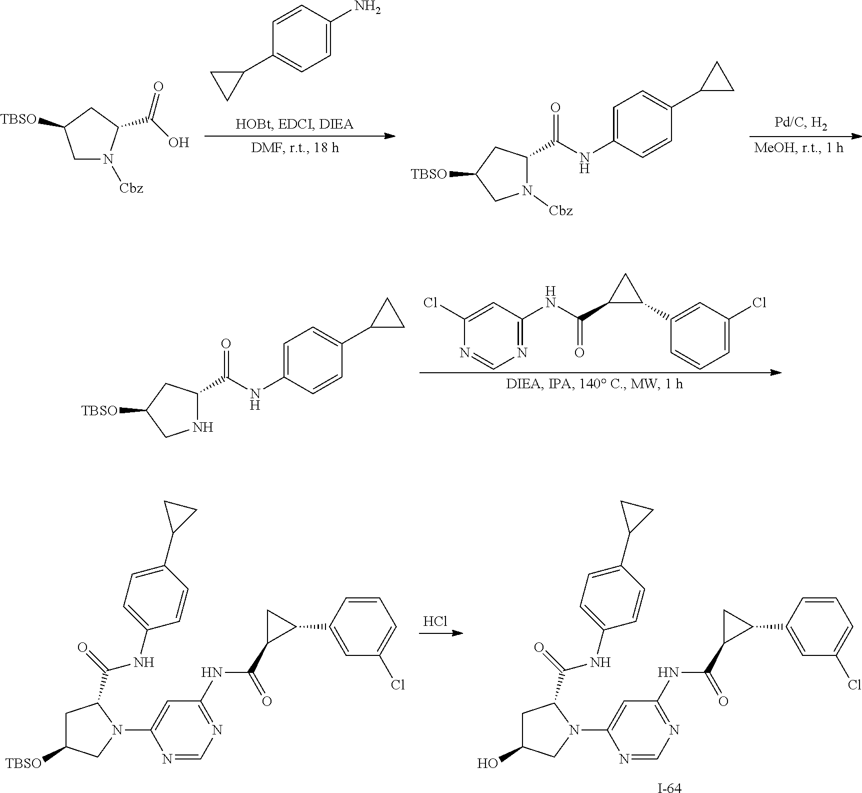

Synthesis of benzyl (2R,4S)-4-((tert-butyldimethylsilyl)oxy)-2-((4-cyclopropyl phenyl)carbamoyl)pyrrolidine-1-carboxylate. A mixture of (2R,4S)-1-((benzyloxy)carbonyl)-4-((tert-butyldimethylsilyl)oxy)pyrrolidine-2-carboxylic acid (500 mg, 1.3 mmol), 4-cyclopropylaniline (213 mg, 1.6 mmol), HOBt (270 mg, 2.0 mmol), EDCI (422 mg, 2.0 mmol) and DIPEA (503 mg, 3.9 mmol) in DMF (5 mL) was stirred at room temperature for 18 h. The reaction was quenched with water (30 mL) and extracted with EtOAc (30 mL×3). The combined organic layers were washed with brine (30 mL), dried over Na₂SO₄ then concentrated to give the crude, which was purified by column chromatography (eluent: PE/EtOAc=5/1) to give benzyl (2R,4S)-4-((tert-butyldimethylsilyl)oxy)-2-((4-cyclopropylphenyl)carbamoyl)pyrrolidine-1-carboxylate (450 mg, 70%) as a yellow solid. ESI-MS [M+H]⁺: 495.3

Synthesis of (2R,4S)-4-((tert-butyldimethylsilyl)oxy)-N-(4-cyclopropylphenyl) pyrrolidine-2-carboxamide. A mixture of 2-(azidomethyl)-6-cyclopropyl-[1,2,4]triazolo[1,5-b]pyridazine (450 mg, 0.91 mmol) and Pd/C (50 mg) in MeOH (5 mL) was stirred at room temperature for 1 h. The reaction mixture was filtered and the filtrate was concentrated in vacuo to give (2R,4S)-4-((tert-butyldimethylsilyl)oxy)-N-(4-cyclopropylphenyl)pyrrolidine-2-carboxamide (300 mg, 91%) as a gray solid which was used in the next step without further purification. ESI-MS [M+H]⁺: 361.2.

Synthesis of (2R,4S)-4-((tert-butyldimethylsilyl)oxy)-1-(6-((1S,2S)-2-(3-chloro phenyl)cyclopropane-1-carboxamido)pyrimidin-4-yl)-N-(4-cyclopropylphenyl)pyrrolidine-2-carboxamide. A mixture of (2R,4S)-4-((tert-butyldimethylsilyl)oxy)-N-(4-cyclopropylphenyl)pyrrolidine-2-carboxamide (100 mg, 0.28 mmol), (1S,2S)-2-(3-chlorophenyl)-N-(6-chloropyrimidin-4-yl)cyclopropane-1-carboxamide (86 mg, 0.28 mmol) and DIPEA (108 mg, 0.84 mmol) in IPA (4 ml) was stirred in a sealed tube. After degassing with N₂ for 1 min, the reaction was irradiated in a microwave reactor at 140° C. for 2 h. After cooling to room temperature, the reaction was quenched with NaHCO₃ (sat. aq., 30 mL) and extracted with DCM (30 mL×3). The combined organic layers were washed with brine (30 mL), dried over Na₂SO₄ and concentrated in vacuo to give the crude, which was purified by preparative TLC (eluent: DCM/MeOH=30/1) to give (2R,4S)-4-((tert-butyldimethylsilyl)oxy)-1-(6-((1S,2S)-2-(3-chlorophenyl)cyclopropane-1-carboxamido)pyrimidin-4-yl)-N-(4-cyclopropylphenyl)pyrrolidine-2-carboxamide (40 mg, 23%) as a white solid. ESI-MS [M+H]⁺: 632.1

Synthesis of (2R,4S)-1-(6-((1S,2S)-2-(3-chlorophenyl)cyclopropane-1-carboxamido)pyrimidin-4-yl)-N-(4-cyclopropylphenyl)-4-hydroxypyrrolidine-2-carboxamide. To a mixture of (2R,4S)-4-((tert-butyldimethylsilyl)oxy)-1-(6-((1S,2S)-2-(3-chlorophenyl)cyclopropane-1-carboxamido)pyrimidin-4-yl)-N-(4-cyclopropylphenyl)pyrrolidine-2-carboxamide (40 mg, 0.063 mmol) in 1,4-dioxane (2 mL) was added HCl (2.0 mL, 4M in 1,4-dioxane). The reaction was stirred at room temperature for 10 h. The reaction was concentrated in vacuo. The residue was neutralized with NH₃ (7 M in MeOH, 2.0 mL) and concentrated in vacuo then purified by preparative TLC (eluent: DCM/MeOH=10/1) to afford (2R,4S)-1-(6-((1S,2S)-2-(3-chlorophenyl)cyclopropane-1-carboxamido)pyrimidin-4-yl)-N-(4-cyclopropylphenyl)-4-hydroxypyrrolidine-2-carboxamide (12 mg, 36%) as a white solid. ESI-MS [M+H]⁺: 518.0. ¹H NMR (400 MHz, MeOD) δ 8.20 (s, 1H), 7.45-7.38 (m, 2H), 7.33 (s, 1H), 7.26 (t, J=7.8 Hz, 1H), 7.22-7.18 (m, 2H), 7.15-7.08 (m, 1H), 7.02-6.98 (d, J=8.5 Hz, 2H), 4.92 (s, 1H), 4.75-4.60 (m, 2H), 3.83-3.80 (m, 1H), 3.48-3.42 (m, 1H), 2.49-2.45 (m, 1H), 2.38-2.32 (m, 1H), 2.27-2.16 (m, 2H), 1.90-1.83 (m, 1H), 1.65-1.58 (m, 1H), 1.39-1.35 (m, 1H), 0.94-0.90 (m, 2H), 0.68-0.58 (m, 2H).

### Example 65

### Synthesis of (2R,4S)-1-(6-((1S,2S)-2-(3-chlorophenyl)cyclopropane-1-carboxamido)pyrimidin-4-yl)-N-(4-cyclopropylphenyl)-4-hydroxy-N-methylpyrrolidine-2-carboxamide (I-65)

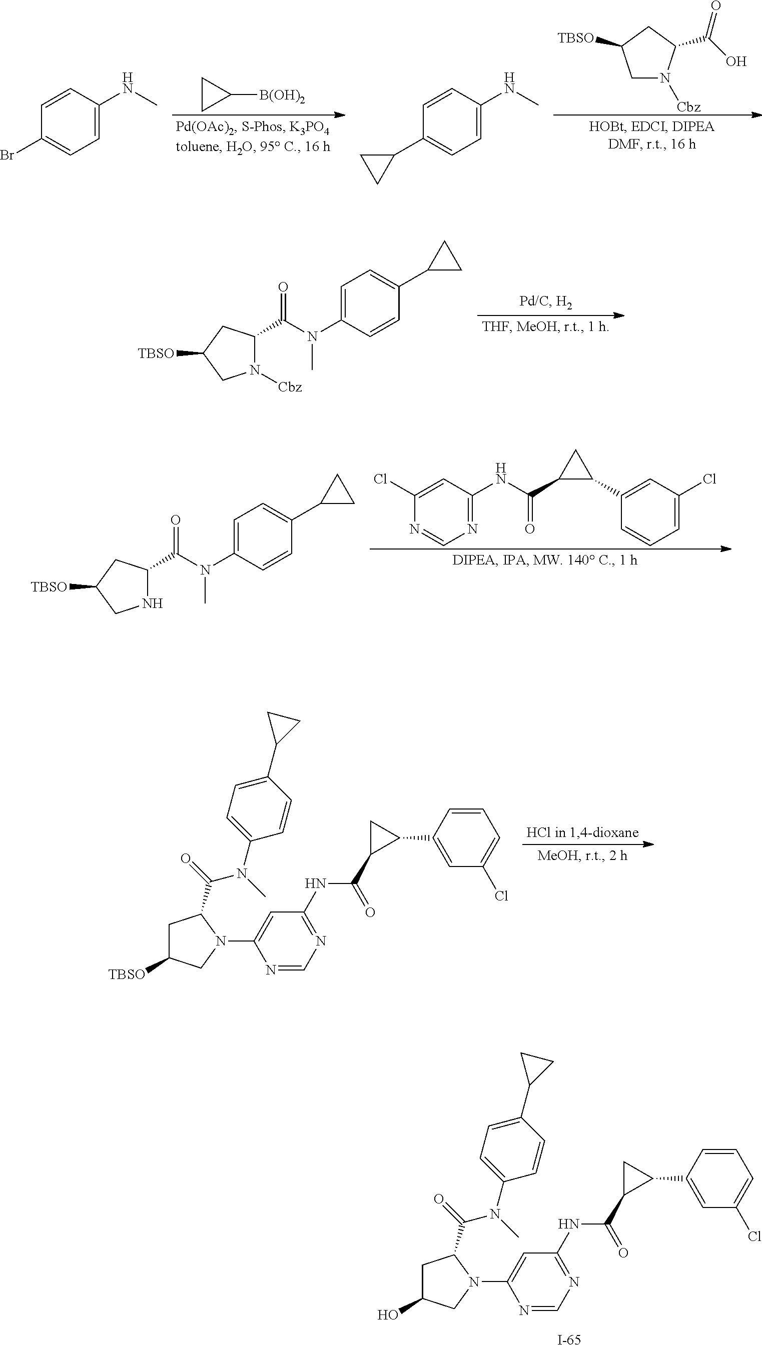

Synthesis of 4-cyclopropyl-N-methylaniline. To a mixture of 4-bromo-N-methylaniline (500 mg, 2.7 mmol), cyclopropylboronic acid (697 mg, 8.1 mmol) and K₃PO₄ (1.72 g, 8.1 mmol) in toluene/H₂O (20 mL/2 mL) was added Pd(OAc)₂ (31 mg, 0.14 mmol) and S-phos (111 mg, 0.27 mmol). The mixture was stirred at 95° C. for 16 h under N₂. The reaction mixture was cooled to room temperature. The reaction mixture was filtered through Celite® and the filter cake was washed with DCM/MeOH (10/1, 50 mL). The filtrate was concentrated in vacuo to give the crude, which was purified by column chromatography (eluent: PE/EtOAc=0˜50%) to give 4-cyclopropyl-N-methylaniline (300 mg, 76%) as a yellow oil. ESI-MS [M+H]⁺: 148.2.

Synthesis of benzyl (2R,4S)-4-((tert-butyldimethylsilyl)oxy)-2-((4-cyclopropylphenyl)(methyl)carbamoyl) pyrrolidine-1-carboxylate. A mixture of (2R,4S)-1-((benzyloxy)carbonyl)-4-((tert-butyldimethylsilyl)oxy)pyrrolidine-2-carboxylic acid (760 mg, 2 mmol), 4-cyclopropyl-N-methylaniline (294 mg, 2 mmol), HOBt (405 mg, 3 mmol), EDCI (576 mg, 3 mmol) and DIPEA (774 mg, 6 mmol) in DMF (10 mL) was stirred at room temperature for 16 h. H₂O (50 mL) was added and the mixture was extracted with EtOAc (30 mL×3). The combined organic layers were washed with brine (30 mL), dried over anhydrous Na₂SO₄, and concentrated in vacuo to give the crude product, which was purified by preparative TLC (eluent: PE/EtOAc=1:1) to give benzyl (2R,4S)-4-((tert-butyldimethylsilyl)oxy)-2-((4-cyclopropylphenyl)(methyl)carbamoyl)pyrrolidine-1-carboxylate (220 mg, 22%) as a yellow oil. ESI-MS [M+H]⁺: 509.2.

Synthesis of (2R,4S)-4-((tert-butyldimethylsilyl)oxy)-N-(4-cyclopropylphenyl)-N-methylpyrrolidine-2-carboxamide. To a mixture of benzyl (2R,4S)-4-((tert-butyldimethylsilyl)oxy)-2-((4-cyclopropylphenyl)(methyl)carbamoyl)pyrrolidine-1-carboxylate (70 mg, 0.14 mmol) in THF (2 mL) and MeOH (2 mL) was added Pd/C (10 mg). The mixture was stirred at room temperature for 1 h under H₂. The reaction mixture was filtered through Celite® and the filter cake was washed with DCM/MeOH (10/1, 20 mL). The filtrate was concentrated in vacuo to give (2R,4S)-4-((tert-butyldimethylsilyl)oxy)-N-(4-cyclopropylphenyl)-N-methylpyrrolidine-2-carboxamide (40 mg, 76%) as a yellow oil. ESI-MS [M+H]⁺: 375.2.

Synthesis of (2R,4S)-4-((tert-butyldimethylsilyl)oxy)-1-(6-((S,2S)-2-(3-chlorophenyl)cyclopropane-1-carboxamido)pyrimidin-4-yl)-N-(4-cyclopropylphenyl)-N-methylpyrrolidine-2-carboxamide. A mixture of (2R,4S)-4-((tert-butyldimethylsilyl)oxy)-N-(4-cyclopropylphenyl)-N-methylpyrrolidine-2-carboxamide (40 mg, 0.11 mmol), (1S,2S)-2-(3-chlorophenyl)-N-(6-chloropyrimidin-4-yl)cyclopropane-1-carboxamide (34 mg, 0.11 mmol) and DIPEA (43 mg, 0.33 mmol) in i-PrOH (2 mL) was stirred in a sealed tube. After degassing with N₂ for 1 min, the reaction was irradiated in microwave at 140° C. for 1 h. The reaction was concentrated in vacuo to give the crude product, which was purified by preparative TLC (eluent: PE/EtOAc=1/2) to give (2R,4S)-4-((tert-butyldimethylsilyl)oxy)-1-(6-((1S,2S)-2-(3-chlorophenyl)cyclopropane-1-carboxamido)pyrimidin-4-yl)-N-(4-cyclopropylphenyl)-N-methylpyrrolidine-2-carboxamide (50 mg, 71%) as a yellow solid. ESI-MS [M+H]⁺: 646.2.

Synthesis of (2R,4S)-1-(6-((1S,2S)-2-(3-chlorophenyl)cyclopropane-1-carboxamido)pyrimidin-4-yl)-N-(4-cyclopropylphenyl)-4-hydroxy-N-methylpyrrolidine-2-carboxamide. To a mixture of (2R,4S)-4-((tert-butyldimethylsilyl)oxy)-1-(6-((1S,2S)-2-(3-chlorophenyl)cyclopropane-1-carboxamido)pyrimidin-4-yl)-N-(4-cyclopropylphenyl)-N-methylpyrrolidine-2-carboxamide (50 mg, 0.08 mmol) in MeOH (2 mL) was added HCl (4M solution in 1,4-dioxane, 2 mL). The mixture was stirred at room temperature for 2 h. The mixture was quenched with NaHCO₃ (sat. aq., 20 mL) and extracted with DCM/MeOH (10/1, 20 mL×3). The combined organic layers were washed with brine (30 mL), dried over anhydrous Na₂SO₄, and concentrated in vacuo to give the crude, which was purified by preparative TLC (eluent: DCM/MeOH=10/1) to give (2R,4S)-1-(6-((1S,2S)-2-(3-chlorophenyl)cyclopropane-1-carboxamido)pyrimidin-4-yl)-N-(4-cyclopropylphenyl)-4-hydroxy-N-methylpyrrolidine-2-carboxamide (33 mg, 80%) as a yellow solid. ESI-MS [M+H]⁺: 532.2. ¹H NMR (400 MHz, DMSO) δ 10.44 (s, 1H), 8.23 (s, 1H), 7.39 (s, 2H), 7.31-7.29 (m, 1H), 7.25-7.23 (m, 2H), 7.20-7.15 (m, 3H), 7.06 (s, 1H), 4.76 (s, 1H), 4.38 (s, 2H), 3.55-3.51 (m, 1H), 3.31 (s, 1H), 3.14 (s, 3H), 2.47-2.39 (m, 2H), 2.06-1.87 (m, 3H), 1.55-1.49 (m, 1H), 1.38-1.34 (m, 1H), 1.00-0.94 (m, 2H), 0.71-0.70 (m, 2H).

### Example 66

### Synthesis of (1S,2S)-2-(4-chloropyridin-2-yl)-N-(6-(((6-cyclopropylimidazo[1,2-a]pyrimidin-2-yl)methyl)amino)pyrimidin-4-yl)cyclopropane-1-carboxamide (I-66)

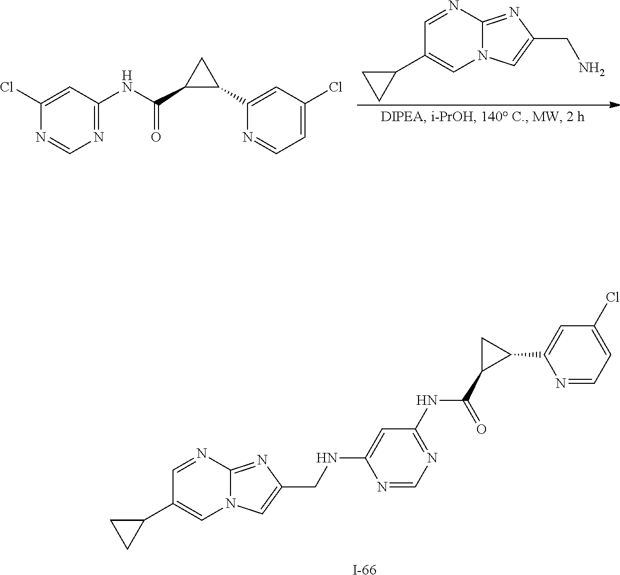

A mixture of (6-cyclopropylimidazo[1,2-a]pyrimidin-2-yl)methanamine (30.0 mg, 0.16 mmol), (1S,2S)-2-(4-chloropyridin-2-yl)-N-(6-chloropyrimidin-4-yl)cyclopropane-1-carboxamide (49.3 mg, 0.16 mmol) and DIPEA (103.2 mg, 0.80 mmol) in i-PrOH (2 mL) was stirred in a sealed tube. After degassing with N₂ for 1 min, the reaction was irradiated in a microwave reactor at 140° C. for 2 h. The reaction was concentrated in vacuo to give the crude product, which was purified by preparative TLC (eluent: DCM/MeOH=10/1) to give (1S,2S)-2-(4-chloropyridin-2-yl)-N-(6-(((6-cyclopropylimidazo[1,2-a]pyrimidin-2-yl)methyl)amino)pyrimidin-4-yl)cyclopropane-1-carboxamide (20 mg, 27%) as a white solid. ESI-MS [M+H]⁺: 461.1. ¹H NMR (400 MHz, DMSO) δ 10.62 (s, 1H), 8.63 (s, 1H), 8.36 (d, J=24 Hz, 2H), 8.15 (s, 1H), 7.90 (s, 1H), 7.61 (s, 1H), 7.53 (s, 1H), 7.32-7.30 (m, 1H), 7.25 (s, 1H), 4.57 (s, 2H), 2.58-2.53 (m, 2H), 1.97-1.91 (m, 1H), 1.51-1.43 (m, 2H), 0.96-0.91 (m, 2H), 0.72-0.68 (m, 2H).

### Example 67

### Synthesis of (S,2S)—N-(6-((4S)-2-(6-(3-azabicyclo[3.1.0]hexan-3-yl)pyridin-3-yl)-4-hydroxypyrrolidin-1-yl)pyrimidin-4-yl)-2-(3-chlorophenyl)cyclopropane-1-carboxamide (I-67)

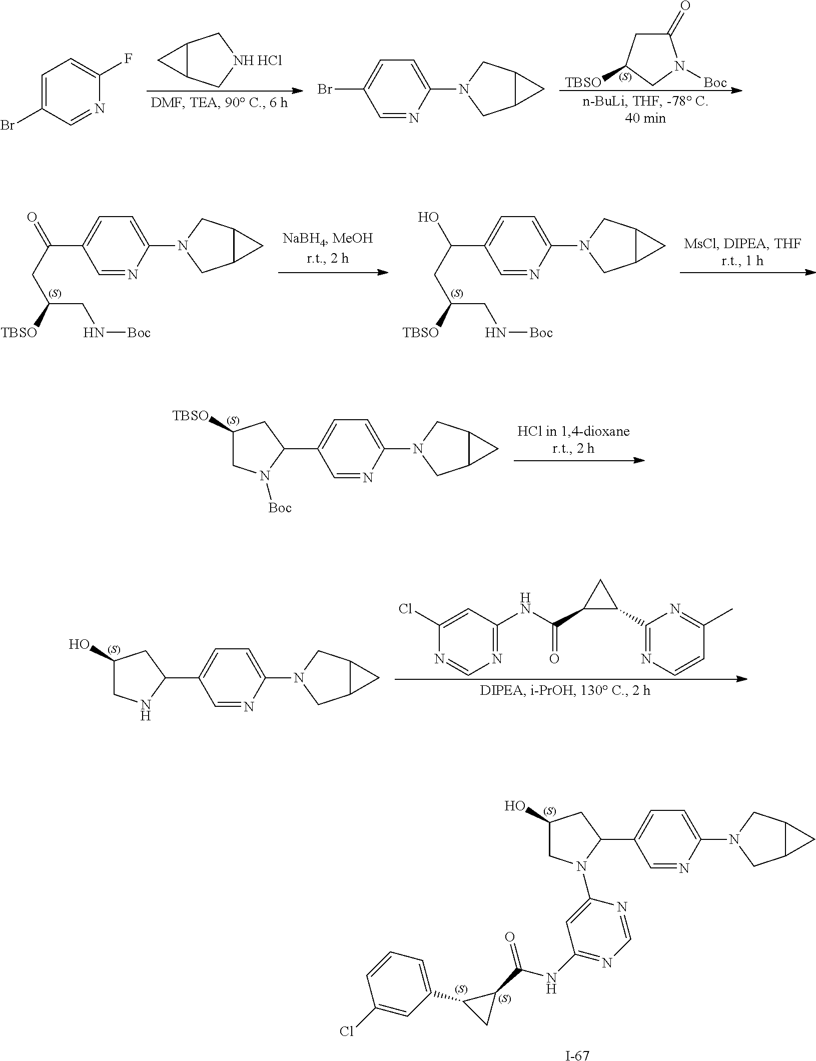

Synthesis of 3-(5-bromopyridin-2-yl)-3-azabicyclo[3.1.0]hexane. A solution of 5-bromo-2-fluoropyridine (1 g, 5.68 mmol) and 3-azabicyclo[3.1.0]hexane hydrogen chloride (1.1 g, 8.52 mmol) and Et₃N (2.87 g, 28.4 mmol) in DMF (20 mL) was stirred at 90° C. for 6 h. The mixture was cooled to room temperature and concentrated in vacuo. The residue was diluted with H₂O (30 mL) and extracted with EtOAc (40 mL×3). The combined organics were washed with brine (50 mL), dried over Na₂SO₄, and concentrated in vacuo. The residue was purified by column chromatography on silica gel, eluting with 0-10% EtOAc in PE to give the product 3-(5-bromopyridin-2-yl)-3-azabicyclo[3.1.0]hexane as a white solid. (970 mg, 72%). ESI-MS [M+H]⁺: 239.2

Synthesis of tert-butyl ((2S)-4-(6-(3-azabicyclo[3.1.0]hexan-3-yl)pyridin-3-yl)-2-((tert-butyldimethylsilyl)oxy)-4-oxobutyl)carbamate. To a solution of 3-(5-bromopyridin-2-yl)-3-azabicyclo[3.1.0]hexane (238 mg, 1 mmol) in THF (10 mL) was added n-BuLi (0.5 mL, 1.2 mmol, 2.4 M solution in hexene) dropwise at −78° C. under N₂ atmosphere. The mixture was stirred at −78° C. for 30 min. The resulting mixture was added dropwise to a solution of tert-butyl (S)-4-((tert-butyldimethylsilyl)oxy)-2-oxopyrrolidine-1-carboxylate (315 mg, 1 mmol) in THF (5.0 mL) at −78° C. The combined mixture was stirred at −78° C. for 10 min. The reaction was quenched with NH₄Cl (sat. eq., 30 mL) and then extracted with EtOAc (50 mL×3). The combined organics were washed with brine (50 mL), dried over Na₂SO₄, and concentrated in vacuo. The residue was purified by column chromatography on silica gel, eluting with 0˜20% EtOAc in PE to give tert-butyl ((2S)-4-(6-(3-azabicyclo[3.1.0]hexan-3-yl)pyridin-3-yl)-2-((tert-butyldimethylsilyl)oxy)-4-oxobutyl)carbamate as a yellow oil. (180 mg, 38%). ESI-MS [M+H]⁺: 476.2

Synthesis of tert-butyl ((2S)-4-(6-(3-azabicyclo[3.1.0]hexan-3-yl)pyridin-3-yl)-2-((tert-butyldimethylsilyl)oxy)-4-hydroxybutyl)carbamate. To a solution of tert-butyl ((2S)-4-(6-(3-azabicyclo[3.1.0]hexan-3-yl)pyridin-3-yl)-2-((tert-butyldimethylsilyl)oxy)-4-oxobutyl)carbamate (180 mg, 0.38 mmol) in MeOH (10 mL) was added NaBH₄ (43 mg, 1.14 mmol). The resulting mixture was stirred at room temperature for 2 h. The reaction was quenched with H₂O (20 mL) and then extracted with EtOAc (30 mL×3). The combined organics were washed with brine (30 mL), dried over Na₂SO₄, and concentrated in vacuo to give tert-butyl ((2S)-4-(6-(3-azabicyclo[3.1.0]hexan-3-yl)pyridin-3-yl)-2-((tert-butyldimethylsilyl)oxy)-4-hydroxybutyl)carbamate (180 mg, crude) as a yellow solid. ESI-MS [M+H]⁺: 478.2

Synthesis of tert-butyl (4S)-2-(6-(3-azabicyclo[3.1.0]hexan-3-yl)pyridin-3-yl)-4-((tert-butyldimethylsilyl)oxy)pyrrolidine-1-carboxylate. To a solution of tert-butyl ((2S)-4-(6-(3-azabicyclo[3.1.0]hexan-3-yl)pyridin-3-yl)-2-((tert-butyldimethylsilyl)oxy)-4-hydroxybutyl)carbamate (180 mg, crude) and Et₃N (30 mg, 0.3 mmol) in DCM (5 mL) was added MsCl (86 mg, 0.75 mmol). The resulting mixture was stirred at room temperature for 1 h. The mixture was concentrated in vacuo. The residue was diluted with H₂O (20 mL) and extracted with DCM (20 mL×3). The combined organics were washed with brine (20 mL), dried over Na₂SO₄, and concentrated in vacuo. The residue was purified by preparative TLC, eluting with 30% EtOAc in PE to give tert-butyl (4S)-2-(6-(3-azabicyclo[3.1.0]hexan-3-yl)pyridin-3-yl)-4-((tert-butyldimethylsilyl)oxy)pyrrolidine-1-carboxylate as a colorless oil. (112 mg, 65% over 2 steps). ESI-MS [M+H]⁺: 460.1

Using a similar procedure, further tert-butyl (4S)-2-(6-(3-azabicyclo[3.1.0]hexan-3-yl)pyridin-3-yl)-4-((tert-butyldimethylsilyl)oxy)pyrrolidine-1-carboxylate was prepared which was then purified by preparative TLC multiple times to give two diasteroisomers: tert-butyl (2R,4S)-2-(6-(3-azabicyclo[3.1.0]hexan-3-yl)pyridin-3-yl)-4-((tert-butyldimethylsilyl)oxy)pyrrolidine-1-carboxylate and tert-butyl (2S,4S)-2-(6-(3-azabicyclo[3.1.0]hexan-3-yl)pyridin-3-yl)-4-((tert-butyldimethylsilyl)oxy)pyrrolidine-1-carboxylate which were used to in the synthesis of further analogues.

Synthesis of (3S)-5-(6-(3-azabicyclo[3.1.0]hexan-3-yl)pyridin-3-yl)pyrrolidin-3-ol. A solution of tert-butyl (4S)-2-(6-(3-azabicyclo[3.1.0]hexan-3-yl)pyridin-3-yl)-4-((tert-butyldimethylsilyl)oxy)pyrrolidine-1-carboxylate (30 mg, 0.065 mmol) in HCl (4.0 N in 1,4-dioxane, 5.0 mL) was stirred at room temperature for 2 h. The mixture was concentrated in vacuo to give (3S)-5-(6-(3-azabicyclo[3.1.0]hexan-3-yl)pyridin-3-yl)pyrrolidin-3-ol (30 mg, crude) as a white solid. ESI-MS [M+H]⁺: 246.1

Synthesis of (S,2S)—N-(6-((4S)-2-(6-(3-azabicyclo[3.1.0]hexan-3-yl)pyridin-3-yl)-4-hydroxypyrrolidin-1-yl)pyrimidin-4-yl)-2-(3-chlorophenyl)cyclopropane-1-carboxamide. A solution of (3S)-5-(6-(3-azabicyclo[3.1.0]hexan-3-yl)pyridin-3-yl)pyrrolidin-3-ol (30 mg, crude), (1S,2S)-2-(3-chlorophenyl)-N-(6-chloropyrimidin-4-yl)cyclopropane-1-carboxamide (30 mg, 0.1 mmol) and DIPEA (42 mg, 0.33 mmol) in iPrOH (3.0 mL) was stirred in a sealed tube. After degassing with N₂ for 1 min, the reaction was irradiated in a microwave reactor at 140° C. for 2 h. The mixture was concentrated in vacuo. The residue was purified by preparative TLC, eluting with 20% MeOH in DCM to give (1S,2S)—N-(6-((4S)-2-(6-(3-azabicyclo[3.1.0]hexan-3-yl)pyridin-3-yl)-4-hydroxypyrrolidin-1-yl)pyrimidin-4-yl)-2-(3-chlorophenyl)cyclopropane-1-carboxamide as a white solid. (15 mg, 45%). ESI-MS [M+H]⁺: 517.2 1H NMR (400 MHz, CDCl₃) δ 8.89-8.77 (m, 1H), 8.17-7.99 (m, 2H), 7.66-7.38 (m, 1H), 7.22-7.16 (m, 2H), 7.08 (s, 1H), 6.99 (d, J=6.7 Hz, 1H), 6.45 (dd, J=27.3, 9.0 Hz, 1H), 5.29-4.95 (m, 2H), 4.65-4.63 (m, 1H), 3.87-3.74 (m, 4H), 3.62-3.54 (m, 2H), 2.59-2.48 (m, 2H), 2.25-2.11 (m, 2H), 1.87 (s, 1H), 1.80-1.50 (m, 3H), 0.88-0.77 (m, 1H), 0.28-0.24 (m, 1H).

### Example 68

### Synthesis of (1S,2S)-2-(4-chloropyridin-2-yl)-N-(6-(((6-cyclopropyl-8-(2,4-dioxoimidazolidin-1-yl)-[1,2,4]triazolo[1,5-a]pyridin-2-yl)methyl)amino)pyrimidin-4-yl)cyclopropane-1-carboxamide (I-68)

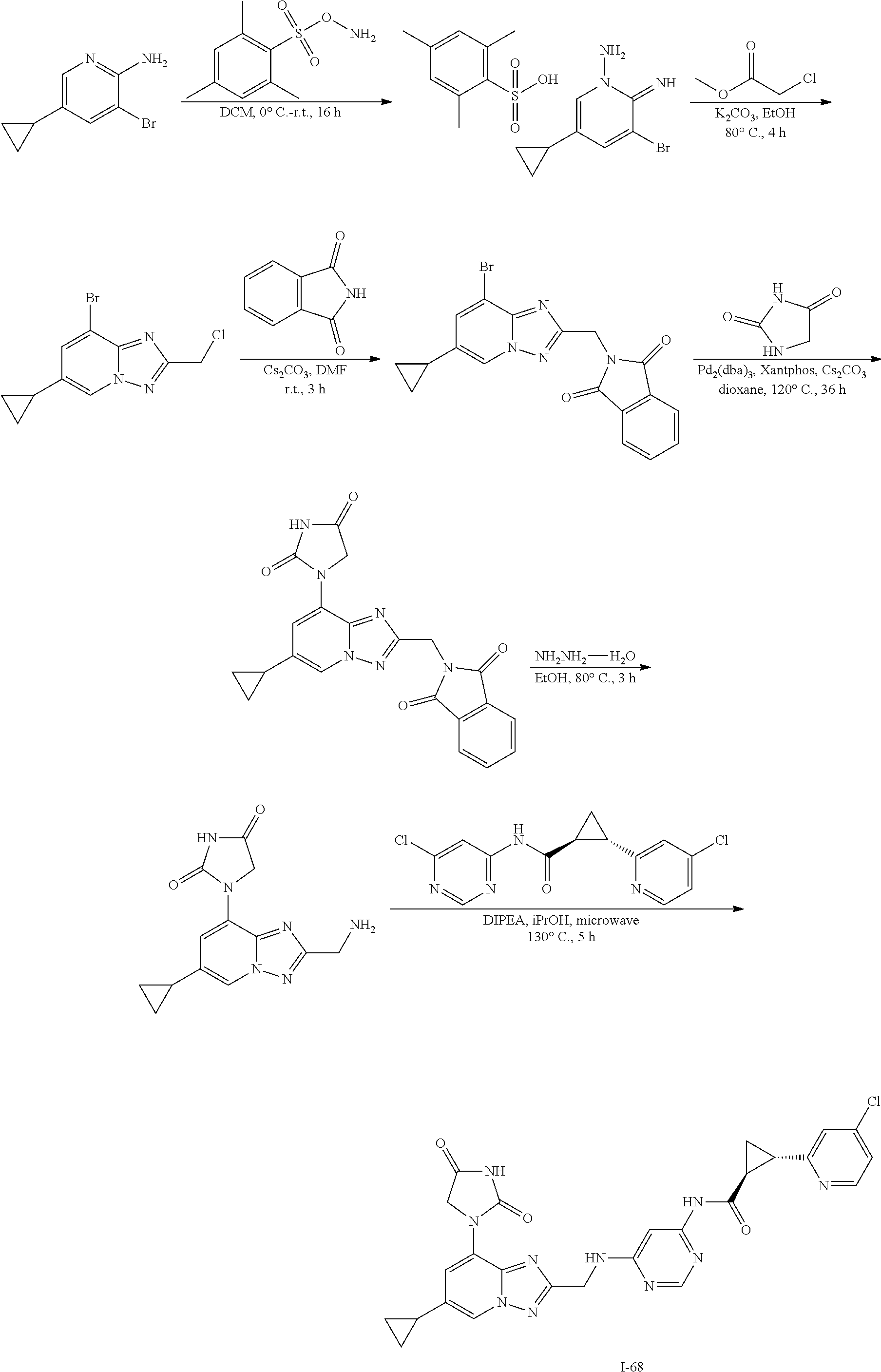

Synthesis of 3-bromo-5-cyclopropyl-2-iminopyridin-1 (2H)-amine 2,4,6-trimethylbenzenesulfonate. To a stirred solution of O-(mesitylsulfonyl) hydroxylamine (16 g, 73 mmol) in DCM (120 mL) was added 3-bromo-5-cyclopropylpyridin-2-amine (4.1 g, 19 mmol) in portions at 0° C. The mixture was stirred at 0° C. for 10 min and warmed to room temperature for 12 h. The reaction mixture was filtered and the solid was dried in vacuo to give 3-bromo-5-cyclopropyl-2-iminopyridin-1 (2H)-amine 2,4,6-trimethylbenzenesulfonate (7.8 g, quant.) as a white solid. ESI-MS [M+H]⁺: 228.0.

Synthesis of 8-bromo-2-(chloromethyl)-6-cyclopropyl-[1,2,4]triazolo[1,5-a]pyridine. The mixture of 3-bromo-5-cyclopropyl-2-iminopyridin-1 (2H)-amine 2,4,6-trimethylbenzenesulfonate (4.0 g, 9.3 mmol), methyl 2-chloroacetate (2.1 g, 19 mmol) and K₂CO₃ (2.6 g, 19 mmol) in EtOH (60 mL) was stirred at 80° C. for 4 h. After cooling to room temperature, the reaction mixture was concentrated in vacuo and diluted with EtOAc (100 mL). The organic layer was washed with water (60 mL) and brine (60 mL), dried over Na₂SO₄ then concentrated in vacuo to give the crude product, which was purified by column chromatography (eluent: EtOAc/PE=1/2) to give 8-bromo-2-(chloromethyl)-6-cyclopropyl-[1,2,4]triazolo[1,5-a]pyridine (1.0 g, 38%) as a yellow solid. ESI-MS [M+H]⁺: 286.1.

Synthesis of 2-((8-bromo-6-cyclopropyl-[1,2,4]triazolo[1,5-a]pyridin-2-yl)methyl)isoindoline-1,3-dione. The mixture of 8-bromo-2-(chloromethyl)-6-cyclopropyl-[1,2,4]triazolo[1,5-a]pyridine (1.0 g, 3.5 mmol), isoindoline-1,3-dione (617 mg, 4.2 mmol) and Cs₂CO₃ (2.3 g, 7.0 mmol) in DMF (15 mL) was stirred at 25° C. for 12 h. The reaction mixture was poured into water (80 mL). The resulting precipitate was collected and dried in vacuo to give 2-((8-bromo-6-cyclopropyl-[1,2,4]triazolo[1,5-a]pyridin-2-yl)methyl)isoindoline-1,3-dione (1.2 g, 86%) as a yellow solid. ESI-MS [M+H]⁺: 397.0.

Synthesis of 2-((6-cyclopropyl-8-(2,4-dioxoimidazolidin-1-yl)-[1,2,4]triazolo[1,5-a]pyridin-2-yl)methyl)isoindoline-1,3-dione. The mixture of 2-((8-bromo-6-cyclopropyl-[1,2,4]triazolo[1,5-a]pyridin-2-yl)methyl)isoindoline-1,3-dione (1.1 g, 2.8 mmol), imidazolidine-2,4-dione (1.4 g, 14 mmol), Pd₂(dba)₃ (512 mg, 0.56 mmol), Xantphos (647 mg, 1.12 mmol) and Cs₂CO₃ (2.7 g, 8.4 mmol) in 1,4-dioxane (30 mL) was stirred at 120° C. for 36 h under N₂. After cooling to room temperature, the reaction mixture was filtered through Celite® and the filter cake was washed with DCM/MeOH (10/1, 50 mL). The filtrate was concentrated in vacuo to give the crude product, which was purified by column chromatography (eluent: EtOAc/PE=1/2) to give 2-((6-cyclopropyl-8-(2,4-dioxoimidazolidin-1-yl)-[1,2,4]triazolo[1,5-a]pyridin-2-yl)methyl)isoindoline-1,3-dione (200 mg, 17%) as a white solid. ESI-MS [M+H]⁺: 417.1.

Synthesis of 1-(2-(aminomethyl)-6-cyclopropyl-[1,2,4]triazolo[1,5-a]pyridin-8-yl)imidazolidine-2,4-dione. The mixture of 2-((6-cyclopropyl-8-(2,4-dioxoimidazolidin-1-yl)-[1,2,4]triazolo[1,5-a]pyridin-2-yl)methyl)isoindoline-1,3-dione (120 mg, 0.29 mmol) and N₂H₄-H₂O (73 mg, 1.45 mmol) in EtOH (4.0 mL) was stirred at 80° C. for 3 h. The reaction mixture was cooled to room temperature and filtered. The filtrate was concentrated and dried in vacuo to give 1-(2-(aminomethyl)-6-cyclopropyl-[1,2,4]triazolo[1,5-a]pyridin-8-yl)imidazolidine-2,4-dione (70 mg, 84%) as a light brown syrup. ESI-MS [M+H]⁺: 287.2.

Synthesis of (1S,2S)-2-(4-chloropyridin-2-yl)-N-(6-(((6-cyclopropyl-8-(2,4-dioxoimidazolidin-1-yl)-[1,2,4]triazolo[1,5-a]pyridin-2-yl)methyl)amino)pyrimidin-4-yl)cyclopropane-1-carboxamide. A mixture of 1-(2-(aminomethyl)-6-cyclopropyl-[1,2,4]triazolo[1,5-a]pyridin-8-yl)imidazolidine-2,4-dione (70 mg, 0.24 mmol), (1S,2S)-2-(4-chloropyridin-2-yl)-N-(6-chloropyrimidin-4-yl)cyclopropane-1-carboxamide (59 mg, 0.19 mmol) and DIPEA (310 mg, 2.4 mmol) in i-PrOH (3.0 mL) was stirred in a sealed tube. After degassing with N₂ for 1 min, the reaction was irradiated in a microwave reactor at 130° C. for 5 h. After cooling to room temperature, the reaction mixture was poured into water (20 mL) and extracted with EtOAc (20 ml×3). The combined organics were washed with brine (20 mL), dried over Na₂SO₄, concentrated to give the crude product, which was purified by preparative TLC (eluent: DCM/MeOH=15/1) to give (1S,2S)-2-(4-chloropyridin-2-yl)-N-(6-(((6-cyclopropyl-8-(2,4-dioxoimidazolidin-1-yl)-[1,2,4]triazolo[1,5-a]pyridin-2-yl)methyl)amino)pyrimidin-4-yl)cyclopropane-1-carboxamide (24 mg, 23%) as a white solid. ESI-MS [M+H]⁺: 559.2. ¹H NMR (400 MHz, DMSO) δ 11.44 (s, 1H), 10.66 (s, 1H), 8.59 (s, 1H), 8.42 (d, J=5.4 Hz, 1H), 8.16 (s, 1H), 8.01 (s, 1H), 7.79 (s, 1H), 7.65 (s, 1H), 7.38-7.32 (m, 2H), 4.85 (s, 2H), 4.71 (s, 2H), 2.65-2.55 (m, 2H), 2.07-2.02 (m, 1H), 1.55-1.46 (m, 2H), 1.02-0.93 (m, 2H), 0.78-0.71 (m, 2H).

### Example 69

### Synthesis of rac-(1S*,2S*)—N-(6-((4S)-2-(6-(3-azabicyclo[3.1.0]hexan-3-yl)pyrin-3-yl)-4-hydroxypyrrolidin-1-yl)pyrimidin-4-yl)-2-(4-methylpyrimidin-2-yl)cyclopropane-1-carboxamide (I-69)

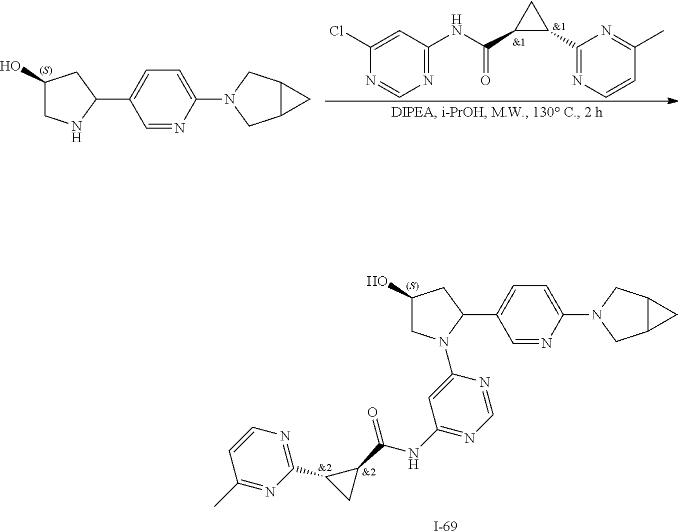

A solution of (3S)-5-(6-(3-azabicyclo[3.1.0]hexan-3-yl)pyridin-3-yl)pyrrolidin-3-ol (69 mg, 0.28 mmol), rac-(1S*,2S*)—N-(6-chloropyrimidin-4-yl)-2-(4-methylpyrimidin-2-yl)cyclopropane-1-carboxamide (81 mg, 0.28 mmol) and DIPEA (181 mg, 1.40 mmol) in iPrOH (3 mL) was stirred in a sealed tube. The reaction was irradiated in a microwave reactor at 130° C. for 2 h. The mixture was cooled to room temperature and concentrated in vacuo. The residue was purified by preparative TLC, eluting with 0-10% MeOH in DCM to give rac-(1S*,2S*)—N-(6-((4S)-2-(6-(3-azabicyclo[3.1.0]hexan-3-yl)pyridin-3-yl)-4-hydroxypyrrolidin-1-yl)pyrimidin-4-yl)-2-(4-methylpyrimidin-2-yl)cyclopropane-1-carboxamide as a white solid. (75 mg, 54%). ESI-MS [M+H]⁺. 499.2. ¹H NMR (400 MHz, CDCl₃) δ 8.65-8.45 (m, 1H), 8.44-8.26 (m, 1H), 8.25-8.08 (m, 1H), 8.06-7.90 (m, 1H), 7.65-7.40 (m, 0.48H), 7.33-7.27 (m, 0.55H), 7.19-7.06 (m, 1H), 6.94 (d, J=4.8 Hz, 1H), 6.41-6.31 (m, 1H), 5.25-4.89 (m, 2H), 4.69-4.50 (m, 1H), 3.81-3.74 (m, 2H), 3.73-3.62 (m, 2H), 3.58-3.35 (m, 2H), 2.76 (s, 1H), 2.50-2.38 (m, 4H), 2.24-2.20 (s, 1H), 2.17-2.03 (m, 1H), 1.73-1.55 (m, 4H), 0.81-0.70 (m, 1H), 0.30-0.20 (m, 1H).

### Example 70

### Synthesis of rac-(1S*,2S*)—N-(6-((2S*,4S)-2-(6-(3-azabicyclo[3.1.0]hexan-3-yl)pyridin-3yl)-4-hydroxypyrrolidin-1-yl)pyrimidin-4-yl)-2-(4-methylpyrimidin-2-yl)cyclopropane-1-carboxamide (I-70

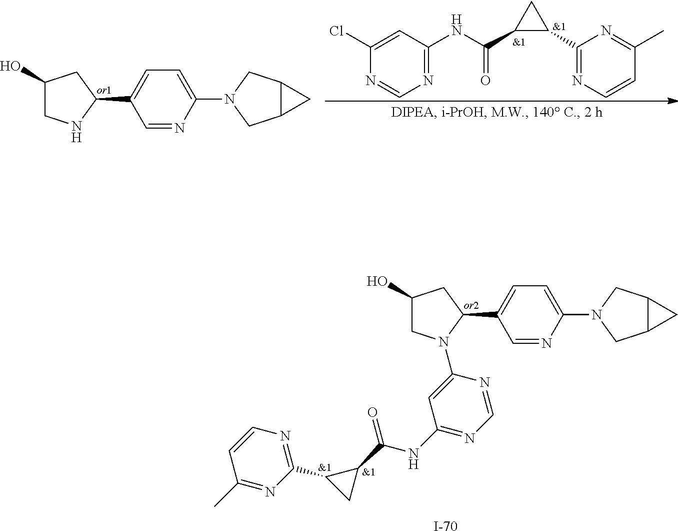

A solution of (3S,5S*)-5-(6-(3-azabicyclo[3.1.0]hexan-3-yl)pyridin-3-yl)pyrrolidin-3-ol (37 mg, 0.15 mmol), rac-(1S*,2S*)—N-(6-chloropyrimidin-4-yl)-2-(4-methylpyrimidin-2-yl)cyclopropane-1-carboxamide (44 mg, 0.15 mmol) and DIPEA (59 mg, 0.46 mmol) in i-PrOH (3 mL) was stirred in a sealed tube. After degassing with N₂ for 1 min, the reaction mixture was irradiated in a microwave reactor at 140° C. for 2 h. The mixture was cooled to room temperature and concentrated in vacuo. The residue was purified by preparative TLC, eluting with 5% MeOH in DCM to give rac-(1S*,2S*)—N-(6-((2S*,4S)-2-(6-(3-azabicyclo[3.1.0]hexan-3-yl)pyridin-3-yl)-4-hydroxypyrrolidin-1-yl)pyrimidin-4-yl)-2-(4-methylpyrimidin-2-yl)cyclopropane-1-carboxamide (15 mg, yield 20%) as a white solid. ESI-MS [M+H]⁺: 499.2 ¹H NMR (400 MHz, CDCl₃) δ 8.42 (t, J=4.8 Hz, 1H), 8.28 (s, 1H), 8.20 (s, 1H), 8.09 (d, J=3.9 Hz, 1H), 7.63-7.58 (m, 1H), 7.20 (s, 1H), 6.98-6.97 (m, 1H), 6.46 (d, J=8.6 Hz, 1H), 5.36 (m, 2H), 4.66 (s, 1H), 3.96-3.70 (m, 3H), 3.65-3.49 (m, 2H), 2.81-2.75 (m, 1H), 2.63-2.54 (m, 1H), 2.46 (d, J=2.9 Hz, 3H), 2.29-2.18 (m, 2H), 1.75-1.51 (m, 4H), 0.88-0.75 (m, 1H), 0.32-0.25 (m, 1H).

### Example 71

### Synthesis of rac-(1S*,2S*)—N-(6-((2R^{•},4S)-2-(6-(3-azabicyclo[3.1.0]hexan-3-yl)pyridin-3-yl)-4-hydroxypyrrolidin-1-yl)pyrimidin-4-yl)-2-(4-methylpyrimidin-2-yl)cyclopropane-1-carboxamide (I-71)

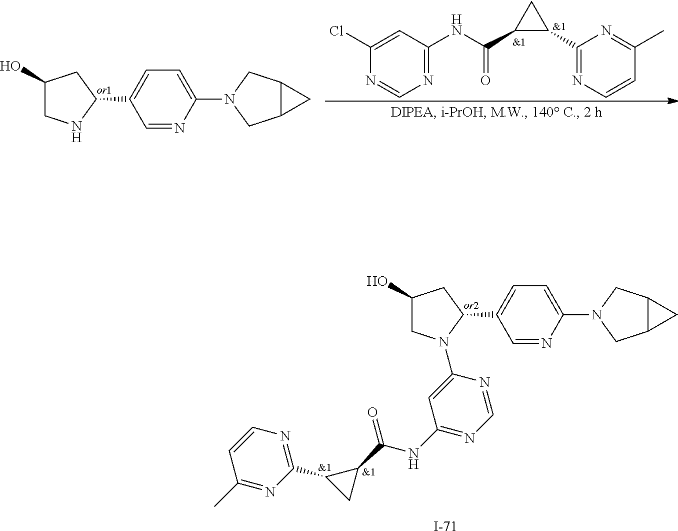

A solution of (3S,5R^{•})-5-(6-(3-azabicyclo[3.1.0]hexan-3-yl)pyridin-3-yl)pyrrolidin-3-ol (40 mg, 0.16 mmol), rac-(1S*,2S*)—N-(6-chloropyrimidin-4-yl)-2-(4-methylpyrimidin-2-yl)cyclopropane-1-carboxamide (47 mg, 0.16 mmol) and DIPEA (63 mg, 0.49 mmol) in i-PrOH (2 mL) was stirred in a sealed tube. After degassing with N₂ for 1 min, the reaction mixture was irradiated in a microwave reactor at 140° C. for 2 h. The mixture was cooled to room temperature and concentrated in vacuo. The residue was purified by preparative TLC, eluting with 5% MeOH in DCM to give rac-(1S*,2S*)—N-(6-((2R^{•},4S)-2-(6-(3-azabicyclo[3.1.0]hexan-3-yl)pyridin-3-yl)-4-hydroxypyrrolidin-1-yl)pyrimidin-4-yl)-2-(4-methylpyrimidin-2-yl)cyclopropane-1-carboxamide (20 mg, yield 25%) as a white solid. ESI-MS [M+H]⁺: 499.3 ¹H NMR (400 MHz, CDCl₃) δ 8.57 (d, J=16.2 Hz, 1H), 8.45-8.39 (m, 1H), 8.19 (s, 1H), 8.03-7.97 (m, 1H), 7.29-7.25 (m, 1H), 7.16 (s, 1H), 6.96 (d, J=5.1 Hz, 1H), 6.31-6.24 (m, 1H), 5.34-4.96 (m, 2H), 4.71-4.52 (m, 1H), 4.02-3.77 (m, 2H), 3.76-3.58 (m, 2H), 3.51-3.32 (m, 2H), 2.90-2.74 (m, 1H), 2.52-2.40 (s, 4H), 2.28-2.09 (m, 2H), 1.75-1.58 (m, 4H), 0.75-0.70 (m, 1H), 0.30-0.27 (m, 1H).

### Example 72

### Synthesis of rac-(1S*,2S*)—N-(6-((2R,4S)-2-(6-cyclopropylimidazo[1,2-a]pyrimidin-2-yl)-4-hydroxypyrrolidin-1-yl)pyrimidin-4-yl)-2-(4-methylpyrimidin-2-yl)cyclopropane-1-carboxamide (I-72)

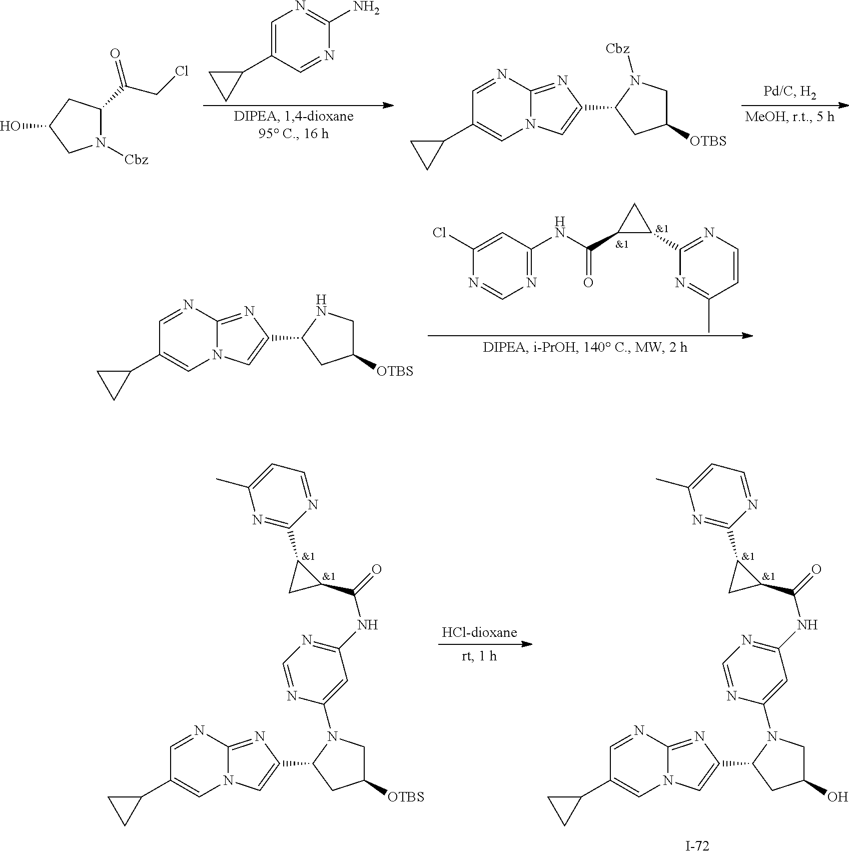

Synthesis of benzyl (2R,4S)-4-((tert-butyldimethylsilyl)oxy)-2-(6-cyclopropylimidazo[1,2-a]pyrimidin-2-yl)pyrrolidine-1-carboxylate. A mixture of benzyl (2R,4S)-4-((tert-butyldimethylsilyl)oxy)-2-(2-chloroacetyl)pyrrolidine-1-carboxylate (150 mg, 0.36 mmol), 5-cyclopropylpyrimidin-2-amine (135 mg, 1.0 mmol) and DIPEA (329 mg, 2.55 mmol) in 1.4-dioxane (10 mL) was stirred at 95° C. for 16 h. The reaction mixture was concentrated in vacuo to give the crude, which was purified by column chromatography (eluent: DCM/MeOH=20/1) to give benzyl (2R,4S)-4-((tert-butyldimethylsilyl)oxy)-2-(6-cyclopropylimidazo[1,2-a]pyrimidin-2-yl)pyrrolidine-1-carboxylate (150 mg, 60%) as a yellow solid. ESI-MS [M+H]⁺: 493.2.

Synthesis of 2-((2R,4S)-4-((tert-butyldimethylsilyl)oxy)pyrrolidin-2-yl)-6-cyclopropylimidazo[1,2-a]pyrimidine. A mixture of benzyl (2R,4S)-4-((tert-butyldimethylsilyl)oxy)-2-(6-cyclopropylimidazo[1,2-a]pyrimidin-2-yl)pyrrolidine-1-carboxylate (100 mg, 0.2 mmol) and Pd/C (20 mg) in MeOH (5 mL) was stirred at room temperature for 5 h under an atmosphere of H₂. The mixture was filtered through Celite® and the filter cake was washed with DCM/MeOH (10/1, 30 mL). The filtrate was concentrated in vacuo to give the crude, which was purified by preparative TLC (eluent: DCM/MeOH=10/1) to give 2-((2R,4S)-4-((tert-butyldimethylsilyl)oxy)pyrrolidin-2-yl)-6-cyclopropylimidazo[1,2-a]pyrimidine (60 mg, 84%) as a yellow oil. ESI-MS [M+H]⁺: 359.2.

Synthesis of rac-(1S*,2S*)—N-(6-((2R,4S)-4-((tert-butyldimethylsilyl)oxy)-2-(6-cyclopropylimidazo[1,2-a]pyrimidin-2-yl)pyrrolidin-1-yl)pyrimidin-4-yl)-2-(4-methylpyrimidin-2-yl)cyclopropane-1-carboxamide. A solution of rac-(1S*,2S*)—N-(6-chloropyrimidin-4-yl)-2-(4-methylpyrimidin-2-yl)cyclopropane-1-carboxamide (73 mg, 0.25 mmol), 2-((2R,4S)-4-((tert-butyldimethylsilyl)oxy)pyrrolidin-2-yl)-6-cyclopropylimidazo[1,2-a]pyrimidine (90 mg, 0.25 mol) and DIPEA (97 mg, 0.75 mol) in i-PrOH (2 mL) was stirred in a sealed tube. After degassing with N₂ for 1 min, the reaction mixture was irradiated in a microwave reactor at 140° C. for 2 h. The mixture was cooled to room temperature and concentrated in vacuo. The residue was purified by preparative TLC, eluting with 10% MeOH in DCM to give (1S*,2S*)—N-(6-((2R,4S)-4-((tert-butyldimethylsilyl)oxy)-2-(6-cyclopropylimidazo[1,2-a]pyrimidin-2-yl)pyrrolidin-1-yl)pyrimidin-4-yl)-2-(4-methylpyrimidin-2-yl)cyclopropane-1-carboxamide (75 mg, 49%) as yellow solid. ESI-MS [M+H]⁺: 612.3

Synthesis of rac-(1S*2S*)—N-(6-((2R,4S)-2-(6-cyclopropylimidazo[1,2-a]pyrimidin-2-yl)-4-hydroxypyrrolidin-1-yl)pyrimidin-4-yl)-2-(4-methylpyrimidin-2-yl)cyclopropane-1-carboxamide. To a reaction mixture of (1S*,2S*)—N-(6-((2R,4S)-4-((tert-butyldimethylsilyl)oxy)-2-(6-cyclopropylimidazo[1,2-a]pyrimidin-2-yl)pyrrolidin-1-yl)pyrimidin-4-yl)-2-(4-methylpyrimidin-2-yl)cyclopropane-1-carboxamide (75 mg, 0.12 mol) in 1,4-dioxane (2 mL) was added HCl (4 N in 1,4-dioxane, 2.0 mL). After stirring at room temperature for 1 h, The resulting mixture was quenched with NaHCO₃(Sat. aq., 20 mL) and extracted with DCM (20×2). The combined organics was washed with brine (20 mL), dried over Na₂SO₄, and concentrated in vacuo. The residue was purified by column chromatography on silica gel, eluting with 0-10% MeOH in DCM to give rac-(1S*,2S*)—N-(6-((2R,4S)-2-(6-cyclopropylimidazo[1,2-a]pyrimidin-2-yl)-4-hydroxypyrrolidin-1-yl)pyrimidin-4-yl)-2-(4-methylpyrimidin-2-yl)cyclopropane-1-carboxamide (39 mg, 64%) as white solid. ESI-MS [M+H]⁺: 498.2. ¹H NMR (400 MHz, MeOD) δ 8.50-8.40 (m, 3H), 8.16 (s, 1H), 7.65 (s, 1H), 7.24 (s, 1H), 7.15 (t, J=4.8 Hz, 1H), 5.51-5.17 (m, 1H), 4.95-4.89 (m, 1H), 4.62-4.60 (m, 1H), 4.02-3.96 (m, 1H), 2.66-2.43 (m, 7H), 2.02-1.95 (m, 1H), 1.70-1.52 (m, 2H), 1.06-1.01 (m, 2H), 0.79-0.75 (m, 2H).

### Example 73

### Synthesis of (1S,2S)-2-(4-chloropyridin-2-yl)-N-(6-((2R,4S)-2-(6-cyclopropylimidazo[1,2-a]pyrimidin-2-yl)-4-hydroxypyrrolidin-1-yl)pyrimidin-4-yl)cyclopropane-1-carboxamide (I-73)

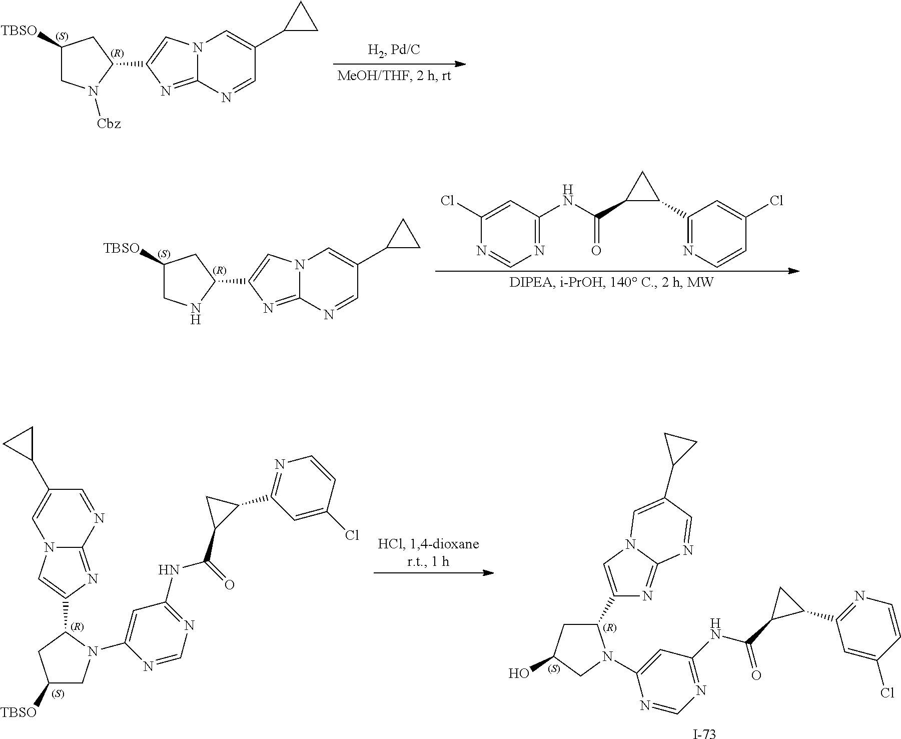

Synthesis of 2-((2R,4S)-4-((tert-butyldimethylsilyl)oxy)pyrrolidin-2-yl)-6-cyclopropylimidazo[1,2-a]pyrimidine. To a mixture of benzyl (2R,4S)-4-((tert-butyldimethylsilyl)oxy)-2-(6-cyclopropylimidazo[1,2-a]pyrimidin-2-yl)pyrrolidine-1-carboxylate (250 mg, 0.51 mmol) in THF/MeOH (5 mL/5 mL) was added Pd/C (30 mg). The reaction mixture was stirred at room temperature for 2 h under H₂. The reaction mixture was filtered through Celite®, and the filter cake was washed with DCM/MeOH (10/1, 20 mL). The filtrate was concentrated in vacuo to give 2-((2R,4S)-4-((tert-butyldimethylsilyl)oxy)pyrrolidin-2-yl)-6-cyclopropylimidazo[1,2-a]pyrimidine (120 mg, 66%) as a yellow oil. ESI-MS [M+H]⁺: 359.2

Synthesis of (1S,2S)—N-(6-((2R,4S)-4-((tert-butyldimethylsilyl)oxy)-2-(6-cyclopropylimidazo[1,2-a]pyrimidin-2-yl)pyrrolidin-1-yl)pyrimidin-4-yl)-2-(4-chloropyridin-2-yl)cyclopropane-1-carboxamide. A mixture of 2-((2R,4S)-4-((tert-butyldimethylsilyl)oxy)pyrrolidin-2-yl)-6-cyclopropylimidazo[1,2-a]pyrimidine (40 mg, 0.11 mmol), (1S,2S)-2-(4-chloropyridin-2-yl)-N-(6-chloropyrimidin-4-yl)cyclopropane-1-carboxamide (34.0 mg, 0.11 mmol) and DIPEA (56.8 mg, 0.44 mmol) in i-PrOH (2 mL) was stirred in a sealed tube. The reaction was irradiated in a microwave reactor at 140° C. for 2 h. The reaction was cooled to room temperature and concentrated in vacuo to give the crude product, which was purified by preparative TLC (eluent: DCM/MeOH=15/1) to give (1S,2S)—N-(6-((2R,4S)-4-((tert-butyldimethylsilyl)oxy)-2-(6-cyclopropylimidazo[1,2-a]pyrimidin-2-yl)pyrrolidin-1-yl)pyrimidin-4-yl)-2-(4-chloropyridin-2-yl)cyclopropane-1-carboxamide (30 mg, 43%) as a white solid. ESI-MS [M+H]⁺: 631.3.

Synthesis of (1S,2S)-2-(4-chloropyridin-2-yl)-N-(6-((2R,4S)-2-(6-cyclopropylimidazo[1,2-a]pyrimidin-2-yl)-4-hydroxypyrrolidin-1-yl)pyrimidin-4-yl)cyclopropane-1-carboxamide. To a mixture of (1S,2S)—N-(6-((2R,4S)-4-((tert-butyldimethylsilyl)oxy)-2-(6-cyclopropylimidazo[1,2-a]pyrimidin-2-yl)pyrrolidin-1-yl)pyrimidin-4-yl)-2-(4-chloropyridin-2-yl)cyclopropane-1-carboxamide (30 mg, 0.048 mmol) in MeOH (2 mL) was added HCl (2 mL, 8.0 mmol, 4 M solution in 1,4-dioxane) at room temperature for 1 h. The reaction mixture was concentrated in vacuo to give a residue, which was neutralized with NH₃ (10 mL, 7 M solution in methanol) and concentrated to give the crude product, which was purified by preparative TLC (eluent: DCM/MeOH=10/1) to give (1S,2S)-2-(4-chloropyridin-2-yl)-N-(6-((2R,4S)-2-(6-cyclopropylimidazo[1,2-a]pyrimidin-2-yl)-4-hydroxypyrrolidin-1-yl)pyrimidin-4-yl)cyclopropane-1-carboxamide (19 mg, 77%) as a white solid. ESI-MS [M+H]⁺: 517.2. ¹H NMR (400 MHz, DMSO) δ 10.74-10.62 (m 1H), 8.65-8.50 (m, 1H), 8.39-8.34 (m, 2H), 8.22-8.04 (m, 1H), 7.62 (s, 1H), 7.49 (d, J=31.3 Hz, 1H), 7.33 (dd, J=5.2, 1.6 Hz, 1H), 7.21-7.09 (m, 1H), 5.43-4.96 (m, 2H), 4.53-4.40 (m, 1H), 3.83-3.60 (m, 2H), 2.66-2.56 (m, 2H), 2.32-2.25 (m, 2H), 1.99-1.92 (m, 1H), 1.56-1.38 (m, 2H), 0.97-0.92 (m, 2H), 0.72-0.67 (m, 2H).

### Example 74

### Synthesis of (1S*,2S*)—N-(6-((2R,4S)-2-(6-cyclopropylimidazo[1,2-a]pyrimidin-2-yl)-4-hydroxypyrrolidin-1-yl)pyrimidin-4-yl)-2-(4-methylpyridin-2-yl)cyclopropane-1-carboxamide, isomer 1 (I-74)

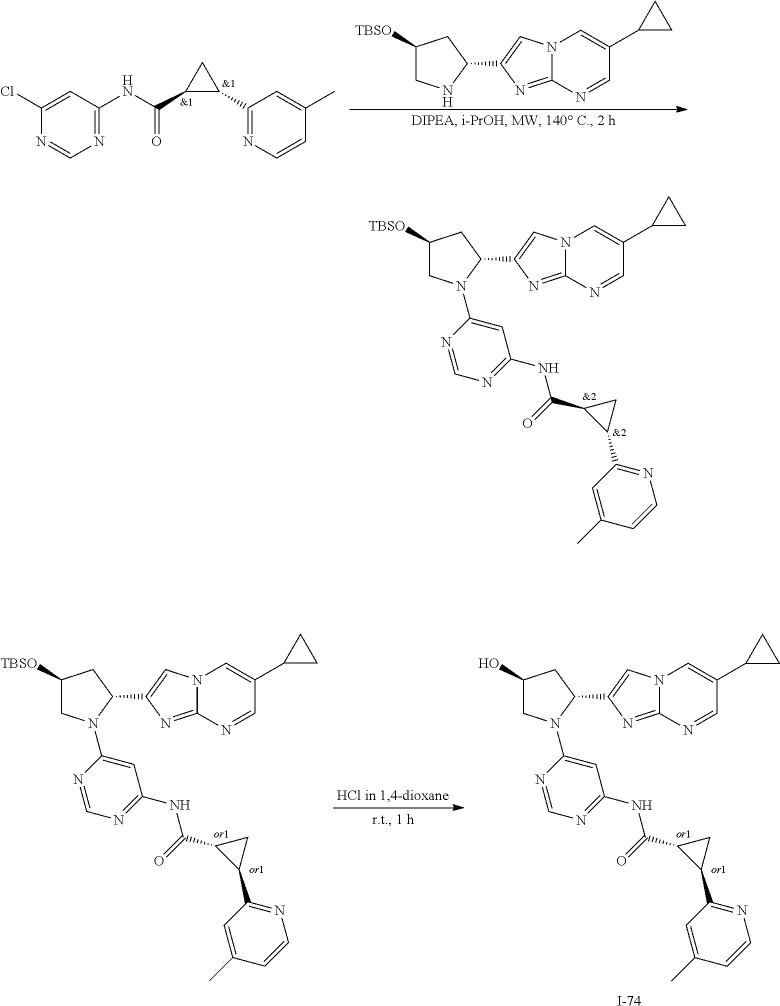

Synthesis of (1S,2S)—N-(6-((2R,4S)-4-((tert-butyldimethylsilyl)oxy)-2-(6-cyclopropylimidazo[1,2-a]pyrimidin-2-yl)pyrrolidin-1-yl)pyrimidin-4-yl)-2-(4-methylpyridin-2-yl)cyclopropane-1-carboxamide & (1R,2R)—N-(6-((2R,4S)-4-((tert-butyldimethylsilyl)oxy)-2-(6-cyclopropylimidazo[1,2-a]pyrimidin-2-yl)pyrrolidin-1-yl)pyrimidin-4-yl)-2-(4-methylpyridin-2-yl)cyclopropane-1-carboxamide. A mixture of rac-(1S*,2S*)—N-(6-chloropyrimidin-4-yl)-2-(4-methylpyridin-2-yl)cyclopropane-1-carboxamide (65 mg, 0.23 mmol), 2-((2R,4S)-4-((tert-butyldimethylsilyl)oxy)pyrrolidin-2-yl)-6-cyclopropylimidazo[1,2-a]pyrimidine (90 mg, 0.25 mmol) and DIPEA (89 mg, 0.69 mmol) in i-PrOH (2 mL) was stirred in a sealed tube. After degassing with N₂ for 1 min, the reaction was irradiated in a microwave reactor at 140° C. for 2 h. The reaction was concentrated in vacuo to give the crude, which was purified by preparative TLC (eluent: DCM/MeOH=20/1) to give the first eluting diastereoisomer (15 mg, 11%) as a yellow solid and the second eluting diastereoisomer (18 mg, 13%) as a yellow solid.

First eluting diastereoisomer: ESI-MS [M+H]⁺: 611.1

Second eluting diastereoisomer: ESI-MS [M+H]⁺: 611.1

Synthesis of (1S*2S*)—N-(6-((2R,4S)-2-(6-cyclopropylimidazo[1,2-a]pyrimidin-2yl)-4-hydroxypyrrolidin-1-yl)pyrimidin-4-yl)-2-(4-methylpyridin-2-yl)cyclopropane-1-carboxamide. To a mixture of (1S*,2S*)—N-(6-((2R,4S)-4-((tert-butyldimethylsilyl)oxy)-2-(6-cyclopropylimidazo[1,2-a]pyrimidin-2-yl)pyrrolidin-1-yl)pyrimidin-4-yl)-2-(4-methylpyridin-2-yl)cyclopropane-1-carboxamide, first eluting diastereoisomer (15 mg, 0.025 mmol) in 1,4-dioxane (1 mL) was added HCl (4 M solution in 1,4-dioxane, 1 mL). The reaction was stirred at room temperature for 1 h. The mixture was quenched with NaHCO₃ (sat. aq., 20 mL) and extracted with DCM/MeOH (10/1, 20 mL×3). The combined organic layers were washed with brine (30 mL), dried over anhydrous Na₂SO₄, and concentrated in vacuo to give the crude, which was purified by preparative TLC (eluent: DCM/MeOH=10/1) to give (1S*,2S*)—N-(6-((2R,4S)-2-(6-cyclopropylimidazo[1,2-a]pyrimidin-2-yl)-4-hydroxypyrrolidin-1-yl)pyrimidin-4-yl)-2-(4-methylpyridin-2-yl)cyclopropane-1-carboxamide (5 mg, 40%) as a white solid. ESI-MS [M+H]⁺: 497.2, ¹H NMR (400 MHz, DMSO) δ 10.72-10.60 (m, 1H), 8.65-8.58 (m, 1H), 8.48-8.12 (m, 3H), 7.55-7.46 (m, 1H), 7.23-7.11 (m, 2H), 7.01 (s, 1H), 5.47-5.02 (m, 2H), 4.54-4.46 (m, 1H), 3.88-3.66 (m, 2H), 2.51-2.46 (m, 2H), 2.32-2.09 (m, 5H), 2.01-1.90 (m, 1H), 1.57-1.36 (m, 2H), 1.07-0.89 (m, 2H), 0.82-0.55 (m, 2H).

### Example 75

### Synthesis of (1S*,2S*)—N-(6-((2R,4S)-2-(6-cyclopropylimidazo[1,2-a]pyrimidin-2-yl)-4-hydroxypyrrolidin-1-yl)pyrimidin-4-yl)-2-(4-methylpyridin-2-yl)cyclopropane-1-carboxamide, isomer 2 (I-75)

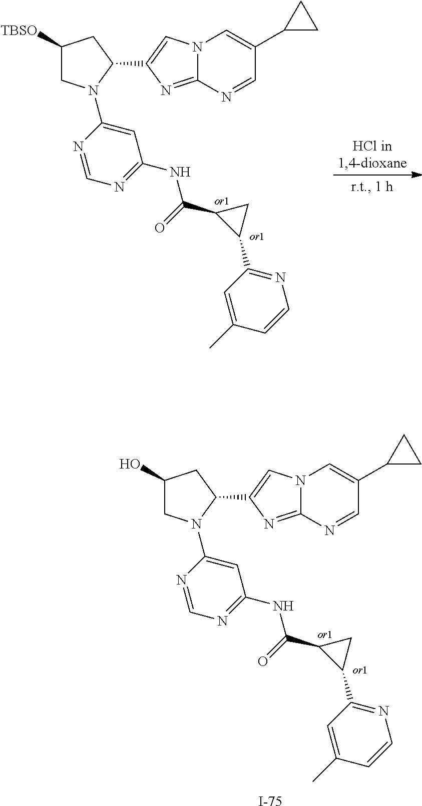

To a mixture of (1S*,2S*)—N-(6-((2R,4S)-4-((tert-butyldimethylsilyl)oxy)-2-(6-cyclopropylimidazo[1,2-a]pyrimidin-2-yl)pyrrolidin-1-yl)pyrimidin-4-yl)-2-(4-methylpyridin-2-yl)cyclopropane-1-carboxamide, second eluting diastereoisomer (18 mg, 0.029 mmol) in 1,4-dioxane (1 mL) was added HCl (4 M solution in 1,4-dioxane, 1 mL). The reaction was stirred at room temperature for 1 h. The mixture was quenched with NaHCO₃ (sat. aq., 20 mL) and extracted with DCM/MeOH (10/1, 20 mL×3). The combined organic layers were washed with brine (30 mL), dried over anhydrous Na₂SO₄, and concentrated in vacuo to give the crude, which was purified by preparative TLC (eluent: DCM/MeOH=10/1) to give (1S*,2S*)—N-(6-((2R,4S)-2-(6-cyclopropylimidazo[1,2-a]pyrimidin-2-yl)-4-hydroxypyrrolidin-1-yl)pyrimidin-4-yl)-2-(4-methylpyridin-2-yl)cyclopropane-1-carboxamide (5 mg, 35%) as a white solid. ESI-MS [M+H]⁺: 497.2, Purity: 91.8% (214 nm), 95.4% (254 nm), ¹H NMR (400 MHz, DMSO) δ 10.72-10.59 (m, 1H), 8.63-8.54 (m, 1H), 8.37-7.99 (m, 3H), 7.55-7.46 (m, 1H), 7.23-7.07 (m, 2H), 7.02-6.98 (m, 1H), 5.47-5.01 (m, 2H), 4.58-4.46 (m, 1H), 3.87-3.63 (m, 2H), 2.51-2.46 (m, 2H), 2.32-2.11 (m, 5H), 2.02-1.92 (m, 1H), 1.55-1.39 (m, 2H), 0.99-0.87 (m, 2H), 0.76-0.69 (m, 2H).

### Example 76

### Synthesis of rac-(1S*,2S*)—N-(6-((2R,4S)-2-(6-cyclopropylimidazo[1,2-a]pyrimidin-2-yl)-4-hydroxypyrrolidin-1-yl)pyrimidin-4-yl)-2-(3-fluoro-4-methylpyridin-2-yl)cyclopropane-1-carboxamide (I-76)

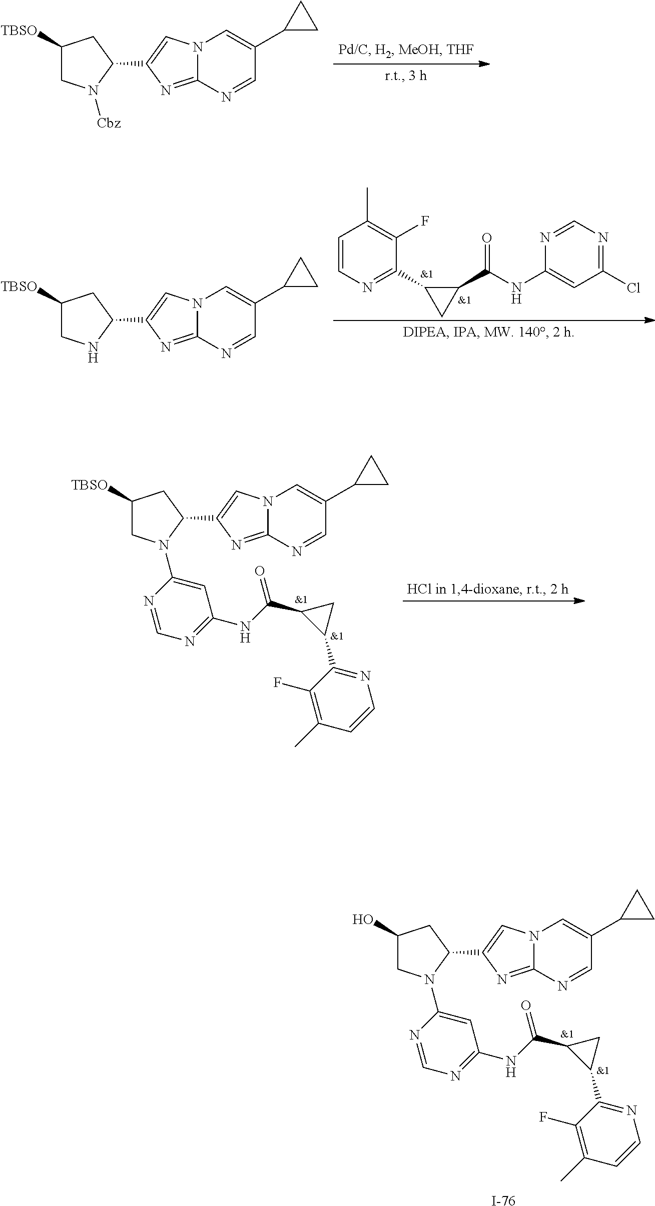

Synthesis of 2-((2R,4S)-4-((tert-butyldimethylsilyl)oxy)pyrrolidin-2-yl)-6-cyclopropylimidazo[1,2-a]pyrimidine. To a mixture of benzyl (2R,4S)-4-((tert-butyldimethylsilyl)oxy)-2-(6-cyclopropylimidazo[1,2-a]pyrimidin-2-yl)pyrrolidine-1-carboxylate (100 mg, 0.2 mmol) in MeOH (2 mL) and THF (2 mL) was added Pd/C (20 mg). The mixture was stirred at room temperature for 3 h under an atmosphere of H₂. The reaction mixture was filtered through Celite® and the filter cake was washed with DCM/MeOH (10/1, 20 mL). The filtrate was concentrated in vacuo to give 2-((2R,4S)-4-((tert-butyldimethylsilyl)oxy)pyrrolidin-2-yl)-6-cyclopropylimidazo[1,2-a]pyrimidine (60 mg, 84%) as a yellow oil. ESI-MS [M+H]⁺: 359.2.

Synthesis of rac-(1S*,2S*)—N-(6-((2R,4S)-4-((tert-butyldimethylsilyl)oxy)-2-(6-cyclopropylimidazo[1,2-a]pyrimidin-2-yl)pyrrolidin-1-yl)pyrimidin-4-yl)-2-(3-fluoro-4-methylpyridin-2-yl)cyclopropane-1-carboxamide. A mixture of 2-((2R,4S)-4-((tert-butyldimethylsilyl)oxy)pyrrolidin-2-yl)-6-cyclopropylimidazo[1,2-a]pyrimidine (51 mg, 0.14 mmol), rac-(1S*,2S*)—N-(6-chloropyrimidin-4-yl)-2-(3-fluoro-4-methylpyridin-2-yl)cyclopropane-1-carboxamide (43 mg, 0.14 mmol) and DIPEA (50 mg, 0.39 mmol) in i-PrOH (2 mL) was stirred in a sealed tube. After degassing with N₂ for 1 min, the reaction was irradiated in a microwave reactor at 140° C. for 2 h. The reaction was concentrated in vacuo to give the crude product, which was purified by preparative TLC (eluent: DCM/MeOH=20/1) to give rac-(1S*,2S*)—N-(6-((2R,4S)-4-((tert-butyldimethylsilyl)oxy)-2-(6-cyclopropylimidazo[1,2-a]pyrimidin-2-yl)pyrrolidin-1-yl)pyrimidin-4-yl)-2-(3-fluoro-4-methylpyridin-2-yl)cyclopropane-1-carboxamide (60 mg, 68%) as a yellow oil. ESI-MS [M+H]⁺: 629.2.

Synthesis of rac-(1S*2S*)—N-(6-((2R,4S)-2-(6-cyclopropylimidazo[1,2-a]pyrimidin-2-yl)-4-hydroxypyrrolidin-1-yl)pyrimidin-4-yl)-2-(3-fluoro-4-methylpyridin-2-yl)cyclopropane-1-carboxamide. To a mixture of rac-(1S*,2S*)—N-(6-((2R,4S)-4-((tert-butyldimethylsilyl)oxy)-2-(6-cyclopropylimidazo[1,2-a]pyrimidin-2-yl)pyrrolidin-1-yl)pyrimidin-4-yl)-2-(3-fluoro-4-methylpyridin-2-yl)cyclopropane-1-carboxamide (60 mg, 0.096 mmol) in MeOH (2 mL) was added HCl (4M solution in 1,4-dioxane, 2 mL). The mixture was stirred at room temperature for 2 h. The mixture was quenched with NaHCO₃ (sat. aq., 30 mL) and extracted with DCM/MeOH (10/1, 20 mL×3). The combined organic layers were washed with brine (30 mL), dried over anhydrous Na₂SO₄, and concentrated in vacuo to give the crude, which was purified by preparative TLC (eluent: DCM/MeOH=10/1) to give rac-(1S*,2S*)—N-(6-((2R,4S)-2-(6-cyclopropylimidazo[1,2-a]pyrimidin-2-yl)-4-hydroxypyrrolidin-1-yl)pyrimidin-4-yl)-2-(3-fluoro-4-methylpyridin-2-yl)cyclopropane-1-carboxamide (35.8 mg, 73%) as a yellow solid. ESI-MS [M+H]⁺: 515.2. ¹H NMR (400 MHz, DMSO) δ 10.78-10.64 (m, 1H), 8.64-8.57 (m, 1H), 8.37 (s, 1H), 8.24-8.15 (m, 2H), 7.58-7.46 (m, 1H), 7.18-7.16 (m, 2H), 5.48-5.03 (m, 2H), 4.56-4.46 (m, 1H), 3.78 (s, 1.5H), 3.22 (s, 0.5H), 2.67-2.55 (m, 2H), 2.32-2.27 (m, 5H), 1.96-1.95 (m, 1H), 1.52-1.51 (m, 2H), 0.97-0.95 (m, 2H), 0.74-0.73 (m, 2H).

### Example 77

### Synthesis of rac-(1S*,2S*)—N-(4-(((6-cyclopropylimidazo[1,2-a]pyrimidin-2yl)methyl)amino)pyrimidin-2-yl)-2-(4-methylpyrimidin-2-yl)cyclopropane-1-carboxamide (I-77)

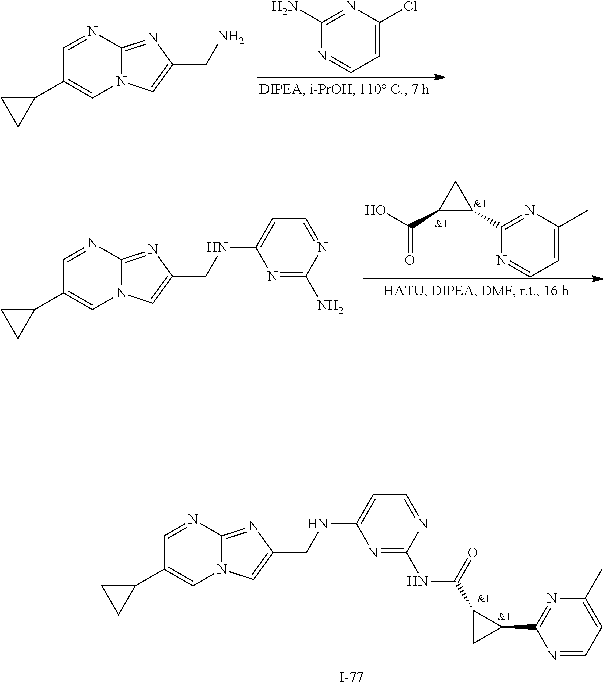

Synthesis of N4-((6-cyclopropylimidazo[1,2-a]pyrimidin-2-yl)methyl)pyrimidine-2,4-diamine. A solution of (6-cyclopropylimidazo[1,2-a]pyrimidin-2-yl)methanamine (50 mg, 0.26 mmol), 4-chloropyrimidin-2-amine (51 mg, 0.39 mmol) and DIPEA (100 mg, 0.78 mmol) in i-PrOH (4 mL) was stirred at 110° C. for 7 h. The mixture was cooled to room temperature and concentrated in vacuo. The residue was diluted with H₂O (20 mL) and extracted with EtOAc (20 mL×2). The combined organics were concentrated in vacuo. The residue was purified by column chromatography on silica gel, eluting with 10˜50% EtOAc in PE to give N₄-((6-cyclopropylimidazo[1,2-a]pyrimidin-2-yl)methyl)pyrimidine-2,4-diamine (15 mg, 21%) as a yellow solid. ESI-MS [M+H]⁺: 282.1

Synthesis of rac-(1S*,2S*)—N-(4-(((6-cyclopropylimidazo[1,2-a]pyrimidin-2-yl)methyl)amino)pyrimidin-2-yl)-2-(4-methylpyrimidin-2-yl)cyclopropane-1-carboxamide. A solution of N4-((6-cyclopropylimidazo[1,2-a]pyrimidin-2-yl)methyl)pyrimidine-2,4-diamine (15 mg, 0.05 mmol) and rac-(1S*,2S*)-2-(4-methylpyrimidin-2-yl)cyclopropane-1-carboxylic acid (34 mg, 0.19 mmol), HATU (27 mg, 0.07 mmol) and DIPEA (32 mg, 0.25 mmol) in DMF (2 mL) was stirred at room temperature for 16 h. The residue was diluted with H₂O (40 mL) and extracted with EtOAc (20 mL×2). The combined organics were washed with brine (20 mL), dried over Na₂SO₄, and concentrated in vacuo. The residue was purified by column chromatography on silica gel, eluting with 0˜5% MeOH in DCM to give rac-(1S*,2S*)—N-(4-(((6-cyclopropylimidazo[1,2-a]pyrimidin-2-yl)methyl)amino)pyrimidin-2-yl)-2-(4-methylpyrimidin-2-yl)cyclopropane-1-carboxamide (3.3 mg, 15%) as a white solid. ESI-MS [M+H]⁺: 442.2, ¹H NMR (400 MHz, MeOD) δ=8.52-8.49 (m, 1H), 8.46-8.41 (m, 2H), 7.92-7.86 (m, 1H), 7.64 (s, 1H), 7.14 (d, J=5.2, 1H), 6.32 (d, J=6.3, 1H), 4.69 (s, 2H), 2.80-2.69 (m, 2H), 2.46-2.43 (m, 3H), 2.02-1.97 (m, 1H), 1.73-1.66 (m, 2H), 1.07-1.02 (m, 2H), 0.80-0.75 (m, 2H).

### Example 78

### Synthesis of rac-(1S*,2S*)—N-(6-(((6-cyclopropyl-[1,2,4]triazolo[1,5-a]pyrimidin-2-yl)methyl)amino)pyrimidin-4-yl)-2-(4-methylpyrimidin-2-yl)cyclopropane-1-carboxamide (I-78)

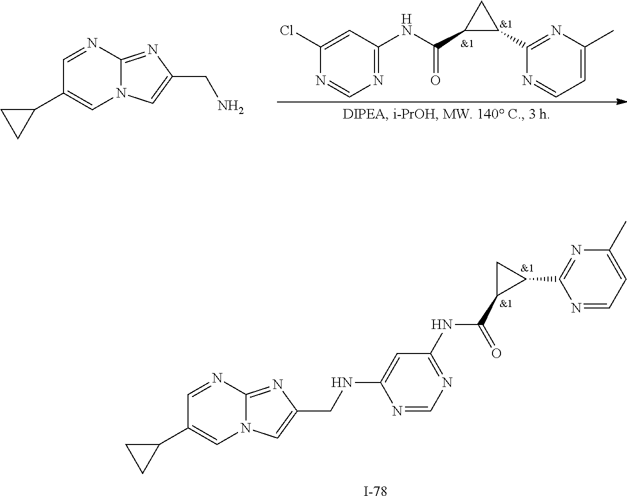

A mixture of (6-cyclopropyl-[1,2,4]triazolo[1,5-a]pyrimidin-2-yl)methanamine (30 mg, 0.16 mmol), rac-(1S*,2S*)—N-(6-chloropyrimidin-4-yl)-2-(4-methylpyrimidin-2-yl)cyclopropane-1-carboxamide (46 mg, 0.16 mmol) and DIPEA (62 mg, 0.48 mmol) in i-PrOH (2 mL) was stirred in a sealed tube. After degassing with N₂ for 1 min, the reaction was irradiated in a microwave reactor at 140° C. for 3 h. The reaction was concentrated in vacuo to give the crude product, which was purified by preparative TLC (eluent: DCM/MeOH=10/1) to give rac-(1S*,2S*)—N-(6-(((6-cyclopropyl-[1,2,4]triazolo[1,5-a]pyrimidin-2-yl)methyl)amino)pyrimidin-4-yl)-2-(4-methylpyrimidin-2-yl)cyclopropane-1-carboxamide (26.5 mg, 37%) as a yellow solid. ESI-MS [M+H]⁺: 443.2. ¹H NMR (400 MHz, DMSO) δ 10.67 (s, 1H), 9.11 (d, J=2.0 Hz, 1H), 8.73 (d, J=2.3 Hz, 1H), 8.52 (d, J=5.1 Hz, 1H), 8.18 (d, J=15.9 Hz, 1H), 8.03 (s, 1H), 7.31 (s, 1H), 7.21 (d, J=5.1 Hz, 1H), 4.74 (s, 2H), 2.65-2.59 (m, 1H), 2.56-2.53 (m, 1H), 2.41 (s, 3H), 2.11-2.04 (m, 1H), 1.58-1.43 (m, 2H), 1.04-0.99 (m, 2H), 0.90-0.86 (m, 2H).

### Example 79

### Synthesis of rac-(1S*,2S*)—N-(6-(((6-(3-azabicyclo[3.1.0]hexan-3-yl)pyridin-3-yl)methyl)amino)pyrimidin-4-yl)-2-(4-methylpyrimidin-2-yl)cyclopropane-1-carboxamide (I-79)

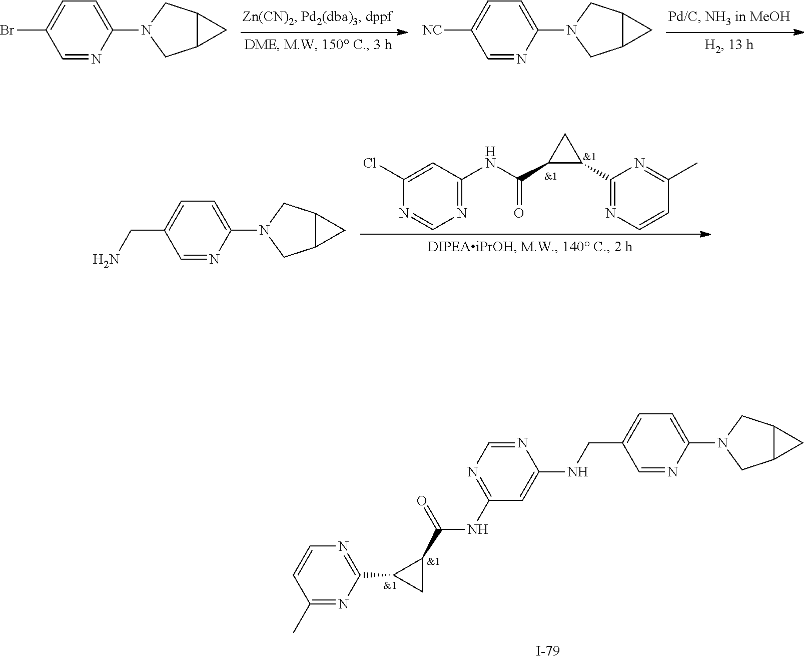

Synthesis of 6-(3-azabicyclo[3.1.0]hexan-3-yl)nicotinonitrile. A solution of 3-(5-bromopyridin-2-yl)-3-azabicyclo[3.1.0]hexane (476 mg, 2.0 mmol), Zn(CN)₂ (470 mg, 4.0 mmol), Pd₂(dba)₃ (110 mg, 0.12 mmol), and dppf (133 mg, 0.24 mmol) in DME (5 mL) was stirred in a sealed tube. After degassing with N₂ for 1 min, the reaction mixture was irradiated in a microwave reactor at 150° C. for 3 h. The mixture was cooled to room temperature, diluted with H₂O (20 mL), and then extracted with EtOAc (30 mL×3). The combined organics was washed with brine (50 mL), dried over Na₂SO₄, and concentrated in vacuo. The residue was purified by column chromatography on silica gel, eluting with 0˜10% EtOAc in PE to give 6-(3-azabicyclo[3.1.0]hexan-3-yl)nicotinonitrile (320 mg, yield 86%) as a yellow solid. ESI-MS [M+H]⁺: 186.1

Synthesis of (6-(3-azabicyclo[3.1.0]hexan-3-yl)pyridin-3-yl)methanamine. To a solution of 6-(3-azabicyclo[3.1.0]hexan-3-yl)nicotinonitrile (100 mg, 0.54 mmol) in NH₃ solution (7 N in MeOH, 10 mL) was added Pd/C (30 mg). The mixture was stirred at room temperature for 13 h under a H₂ atmosphere. The reaction mixture was filtered by Celite®, and the filtrate was concentrated in vacuo to give (6-(3-azabicyclo[3.1.0]hexan-3-yl)pyridin-3-yl)methanamine (100 mg, crude) as a yellow oil, which was used in the next step. ESI-MS [M+H]⁺: 190.2

Synthesis of rac-(1S*,2S*)—N-(6-(((6-(3-azabicyclo[3.1.0]hexan-3-yl)pyridin-3-yl)methyl)amino)pyrimidin-4-yl)-2-(4-methylpyrimidin-2-yl)cyclopropane-1-carboxamide. A solution of (6-(3-azabicyclo[3.1.0]hexan-3-yl)pyridin-3-yl)methanamine (50 mg, crude), N-rac-(1S*,2S*)—N-(6-chloropyrimidin-4-yl)-2-(4-methylpyrimidin-2-yl)cyclopropane-1-carboxamide (75 mg, 0.26 mmol) and DIPEA (101 mg, 0.78 mmol) in i-PrOH (3 mL) was stirred in a sealed tube. After degassing with N₂ for 1 min, the reaction mixture was irradiated in a microwave reactor at 140° C. for 2 h. The mixture was cooled to room temperature and concentrated in vacuo. The residue was purified by prep-TLC, eluting with 10% MeOH in DCM to give rac-(1S*,2S*)—N-(6-(((6-(3-azabicyclo[3.1.0]hexan-3-yl)pyridin-3-yl)methyl)amino)pyrimidin-4-yl)-2-(4-methylpyrimidin-2-yl)cyclopropane-1-carboxamide (16 mg, yield 14%) as white solid. ESI-MS [M+H]⁺: 443.2, ¹H NMR (400 MHz, DMSO) δ 10.65 (s, 1H), 8.52 (d, J=5.1 Hz, 1H), 8.18 (s, 1H), 7.99 (d, J=1.8 Hz, 1H), 7.74 (t, J=5.7 Hz, 1H), 7.47-7.34 (m, 1H), 7.20 (d, J=5.0 Hz, 2H), 6.39 (d, J=8.6 Hz, 1H), 4.31 (s, 2H), 3.60 (d, J=10.1 Hz, 2H), 3.27-3.22 (m, 2H), 2.73-2.59 (m, 2H), 2.41 (s, 3H), 1.72-1.58 (m, 2H), 1.56-1.39 (m, 2H), 0.76-0.58 (m, 1H), 0.20-0.10 (m, 1H).

### Examples 80-82

### Synthesis of rac-(1S*,2S*)—N-(6-(((6-cyclopropyl-8-(3-methyl-2,4-dioxoimidazolidin-1-yl)imidazo[1,2-a]pyrazin-2-yl)methyl)amino)pyrimidin-4-yl)-2-(4-methylpyrimidin-2-yl)cyclopropane-1-carboxamide (1-80)

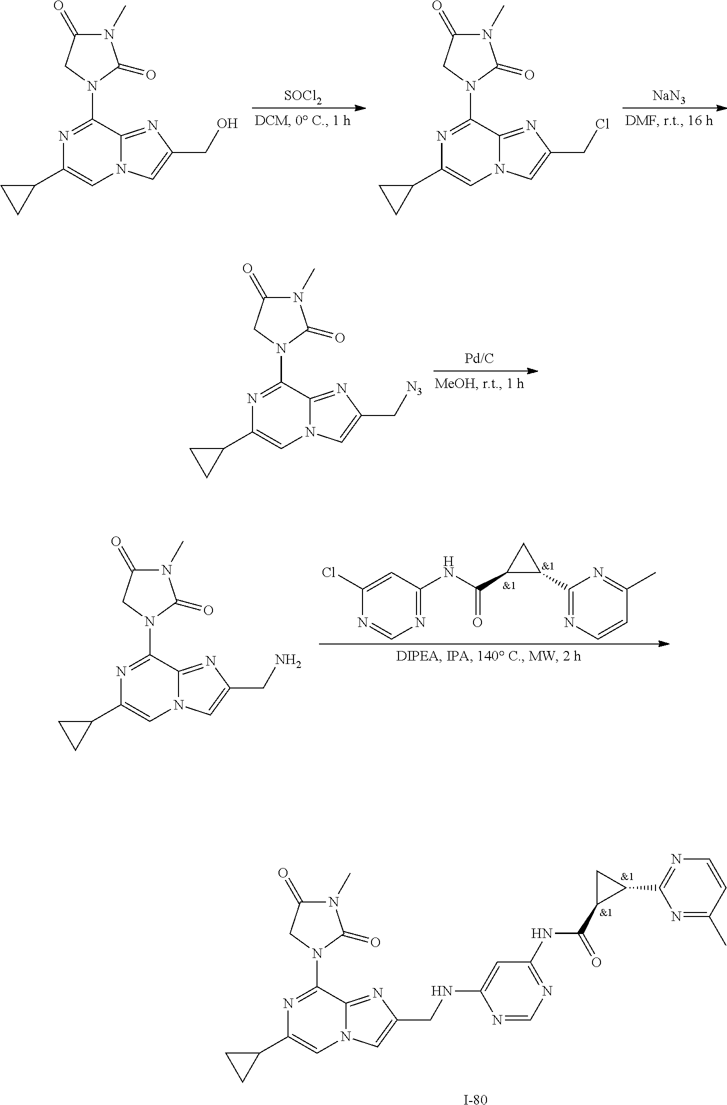

Synthesis of 1-(2-(chloromethyl)-6-cyclopropylimidazo[1,2-a]pyrazin-8-yl)-3-methylimidazolidine-2,4-dione. To the mixture of 1-(6-cyclopropyl-2-(hydroxymethyl)imidazo[1,2-a]pyrazin-8-yl)-3-methylimidazolidine-2,4-dione (230 mg, 0.76 mmol) in DCM (5.0 mL) was added SOCl₂ (0.5 mL) at 0° C. The mixture was stirred for 1 h at 0° C. then concentrated in vacuo to afford 1-(2-(chloromethyl)-6-cyclopropylimidazo[1,2-a]pyrazin-8-yl)-3-methylimidazolidine-2,4-dione (250 mg, crude) as yellow oil. ESI-MS [M+H]⁺: 320.1.

Synthesis of 1-(2-(azidomethyl)-6-cyclopropylimidazo[1,2-a]pyrazin-8-yl)-3-methylimidazolidine-2,4-dione. To the mixture of 1-(2-(chloromethyl)-6-cyclopropylimidazo[1,2-a]pyrazin-8-yl)-3-methylimidazolidine-2,4-dione (250 mg, crude) in DMF (5.0 mL) was added NaN₃ (97.5 mg, 1.5 mmol). The reaction mixture was stirred at room temperature for 16 h. The reaction was diluted with H₂O (50 mL) and extracted with EtOAc (30 mL×3). The combined organic layers were washed with brine (20 mL), dried over anhydrous Na₂SO₄ and concentrated in vacuo to give 1-(2-(azidomethyl)-6-cyclopropylimidazo[1,2-a]pyrazin-8-yl)-3-methylimidazolidine-2,4-dione (250 mg, crude) as yellow oil. ESI-MS [M+H]⁺: 327.1.

Synthesis of 1-(2-(aminomethyl)-6-cyclopropylimidazo[1,2-a]pyrazin-8-yl)-3-methylimidazolidine-2,4-dione. A mixture of 1-(2-(azidomethyl)-6-cyclopropylimidazo[1,2-a]pyrazin-8-yl)-3-methylimidazolidine-2,4-dione (250 mg, crude) in MeOH (10 mL) was added Pd/C (50 mg) was stirred at room temperature under a H₂ atmosphere. The reaction mixture was filtered through Celite® and the filter cake was washed with MeOH (30 mL) and concentrated in vacuo to give the crude, which was purified with preparative TLC (eluent: DCM/MeOH=8/1) to afford 1-(2-(aminomethyl)-6-cyclopropylimidazo[1,2-a]pyrazin-8-yl)-3-methylimidazolidine-2,4-dione (180 mg, 79% over 3 steps) as yellow oil. ESI-MS [M+H]⁺: 301.2.

Synthesis of rac-(1S*2S*)—N-(6-(((6-cyclopropyl-8-(3-methyl-2,4-dioxoimidazolidin-1-yl)imidazo[1,2-a]pyrazin-2-yl)methyl)amino)pyrimidin-4-yl)-2-(4-methylpyrimidin-2-yl)cyclopropane-1-carboxamide. A mixture of 1-(2-(aminomethyl)-6-cyclopropylimidazo[1,2-a]pyrazin-8-yl)-3-methylimidazolidine-2,4-dione (90 mg, 0.30 mmol), rac-(1S*,2S*)—N-(6-chloropyrimidin-4-yl)-2-(4-methylpyrimidin-2-yl)cyclopropane-1-carboxamide (104 mg, 0.36 mmol) and DIPEA (116.1 mg, 0.90 mmol) in iPrOH (2 mL) was stirred in a sealed tube. The reaction was irradiated in a microwave reactor at 140° C. for 2 h. After cooling to room temperature, the reaction was concentrated in vacuo to give the crude, which was purified with preparative TLC (DCM/MeOH=10/1) to afford rac-(1S*,2S*)—N-(6-(((6-cyclopropyl-8-(3-methyl-2,4-dioxoimidazolidin-1-yl)imidazo[1,2-a]pyrazin-2-yl)methyl)amino)pyrimidin-4-yl)-2-(4-methylpyrimidin-2-yl)cyclopropane-1-carboxamide (24 mg, 14% ) as a yellow solid (mixture of enantiomers). ESI-MS [M+H]⁺: 554.2, ¹H NMR (400 MHz, DMSO) δ 10.70 (s, 1H), 8.52 (d, J=5.1 Hz, 1H), 8.35 (s, 1H), 8.19 (s, 1H), 7.97 (s, 1H), 7.86 (s, 1H), 7.31 (s, 1H), 7.21 (d, J=5.1 Hz, 1H), 4.89 (s, 2H), 4.63 (s, 2H), 2.97 (s, 3H), 2.64-2.60 (m, 1H), 2.45-2.41 (m, 4H), 2.03-1.97 (m, 1H), 1.56-1.49 (m, 2H), 0.88-0.86 (m, 4H).

The mixture was separated using SFC 80 (Daicel CHIRALPAK OJ-H 250 mm×20 mm CO₂/MeOH (0.2% NH₄·OH)=54/46.45 g/min. 35° C.) to give two enantiomers: (1R,2R)—N-(6-(((6-cyclopropyl-8-(3-methyl-2,4-dioxoimidazolidin-1-yl)imidazo[1,2-a]pyrazin-2-yl)methyl)am ino)pyrimidin-4-yl)-2-(4-methylpyrimidin-2-yl)cyclopropane-1-carboxamide, and (1S,2S)—N-(6-(((6-cyclopropyl-8-(3-methyl-2,4-dioxoimidazolidin-1-yl)imidazo[1,2-a]pyrazin-2-yl)methyl)amino)pyrimidin-4-yl)-2-(4-methylpyrimidin-2-yl)cyclopropane-1-carboxamide.

First eluting enantiomer (I-81): ESI-MS [M+H]⁺: 554.2. ¹H NMR (400 MHz, DMSO) δ 10.69 (s, 1H), 8.52 (d, J=5.1 Hz, 1H), 8.35 (s, 1H), 8.19 (s, 1H), 7.96 (s, 1H), 7.86 (s, 1H), 7.31 (s, 1H), 7.21 (d, J=5.1 Hz, 1H), 4.89 (s, 2H), 4.63 (s, 2H), 2.97 (s, 3H), 2.64-2.60 (m, 1H), 2.50-2.41 (m, 4H), 2.00-1.97 m, 1H), 1.55-1.48 (m, 2H), 0.88-0.86 (m, 4H). RT=3.0 min, 100.0% e.e.

Second eluting enantiomer (I-82): ESI-MS [M+H]⁺: 554.2. ¹H NMR (400 MHz, DMSO) δ 10.69 (s, 1H), 8.52 (d, J=5.1 Hz, 1H), 8.35 (s, 1H), 8.19 (s, 1H), 7.96 (s, 1H), 7.86 (s, 1H), 7.31 (s, 1H), 7.21 (d, J=5.1 Hz, 1H), 4.89 (s, 2H), 4.63 (s, 2H), 2.97 (s, 3H), 2.64-2.60 (m, 1H), 2.45-2.41 (m, 4H), 1.98-1.97 (m, 1H), 1.49-1.46 (m, 2H), 0.88-0.83 (m, 4H). RT=3.67 min, 96.0% e.e.

### Example 83

### Synthesis of (1S,2S)—N-(6-(((6-cyclopropyl-8-(3-methyl-2,4-dioxoimidazolidin-1-yl)imidazo[1,2-a]pyrazin-2-yl)methyl)(methyl)amino)pyrimidin-4-yl)-2-(4-methylpyrimidin-2-yl)cyclopropane-1-carboxamide (I-83)

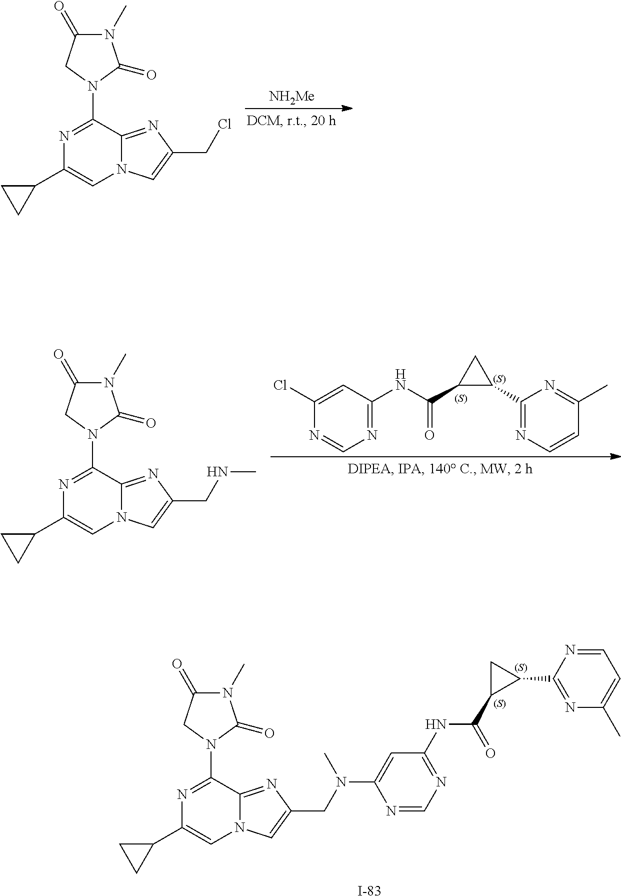

Synthesis of 1-(6-cyclopropyl-2-((methylamino)methyl)imidazo[1,2-a]pyrazin-8-yl)-3-methylimidazolidine-2,4-dione. To a mixture of 1-(2-(chloromethyl)-6-cyclopropylimidazo[1,2-a]pyrazin-8-yl)-3-methylimidazolidine-2,4-dione (100 mg, 0.31 mmol) in DCM (5 mL) was added MeNH₂ (0.8 mL, 1.6 mmol, 2M solution in THF). The reaction was stirred for 20 h at room temperature then was concentrated in vacuo to give the crude, which was purified by preparative TLC (DCM/MeOH=10/1) to afford 1-(6-cyclopropyl-2-((methylamino)methyl)-imidazo[1,2-a]pyrazin-8-yl)-3-methylimidazolidine-2,4-dione (20 mg, 20%) as a yellow solid. ESI-MS [M+H]⁺: 315.2.

Synthesis of (1S,2S)—N-(6-(((6-cyclopropyl-8-(3-methyl-2,4-dioxoimidazolidin-1-yl)imidazo[1,2-a]pyrazin-2-yl)methyl)(methyl)amino)pyrimidin-4-yl)-2-(4-methylpyrimidin-2-yl)cyclopropane-1-carboxamide. To the mixture of 1-(6-cyclopropyl-2-((methylamino)methyl)imidazo[1,2-a]pyrazin-8-yl)-3-methylimidazolidine-2,4-dione (20 mg, 0.064 mmol), (1S,2S)—N-(6-chloropyrimidin-4-yl)-2-(4-methylpyrimidin-2-yl)cyclopropane-1-carboxamide (18 mg, 0.064 mmol) and DIPEA (25 mg, 0.19 mmol) in IPA (1 mL) was stirred in a sealed tube. The reaction was irradiated in a microwave reactor at 140° C. for 2 h. After cooling to room temperature, the reaction was concentrated in vacuo to give the crude product, which was purified with preparative TLC (DCM/MeOH=10/1) to afford (1S,2S)—N-(6-(((6-cyclopropyl-8-(3-methyl-2,4-dioxoimidazolidin-1-yl)imidazo[1,2-a]pyrazin-2-yl)methyl)(methyl)amino)pyrimidin-4-yl)-2-(4-methylpyrimidin-2-yl)cyclopropane-1-carboxamide (3.5 mg, 10%) as a yellow solid. ESI-MS [M+H]⁺:568.3. ¹H NMR (400 MHz, DMSO) δ 10.80 (s, 1H), 8.52 (d, J=5.1 Hz, 1H), 8.29 (d, J=14.5 Hz, 2H), 7.85 (s, 1H), 7.45 (s, 1H), 7.21 (d, J=5.1 Hz, 1H), 4.89 (s, 4H), 3.12 (s, 3H), 2.96 (s, 3H), 2.65-2.62 (m, 1H), 2.56-2.54 (m, 1H), 2.41 (s, 3H), 2.03-1.97 (m, 1H), 1.56-1.49 (m, 2H), 0.87-0.86 (m, 4H).

### Example 84

### Synthesis of rac-(1S*,2S*)—N-(4-(((6-cyclopropyl-8-(3-methyl-2,4-dioxoimidazolidin-1-yl) imidazo[1,2-a]pyrazin-2-yl)methyl)amino)-5-fluoropyrimidin-2-yl)-2-(4-methylpyrimidin-2-yl) cyclopropane-1-carboxamide (I-84)

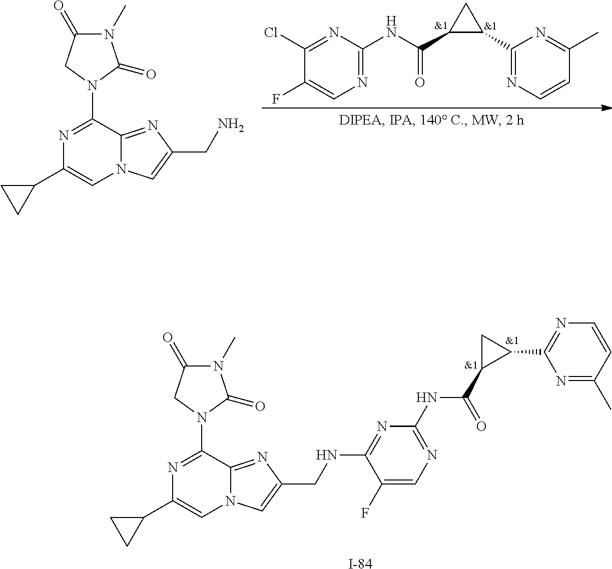

A mixture of 1-(2-(aminomethyl)-6-cyclopropylimidazo[1,2-a]pyrazin-8-yl)-3-methylimidazolidine-2,4-dione (80 mg, 0.27 mmol), (1S,2S)—N-(4-chloro-5-fluoropyrimidin-2-yl)-2-(4-methylpyrimidin-2-yl)cyclopropane-1-carboxamide (83 mg, 0.27 mmol) and DIPEA (142 mg, 1.1 mmol) in i-PrOH (3 mL) was stirred in a sealed tube. The reaction was irradiated in a microwave reactor at 140° C. for 2 h. The reaction was cooled to room temperature then concentrated in vacuo to give the crude, which was purified by preparative TLC (eluent: DCM/MeOH=10/1) to give rac-(1S* ,2S*)—N-(4-(((6-cyclopropyl-8-(3-methyl-2,4-dioxoimidazolidin-1-yl)imidazo[1,2-a]pyrazin-2-yl)methyl)amino)-5-fluoropyrimidin-2-yl)-2-(4-methylpyrimidin-2-yl)cyclopropane-1-carboxamide (15.6 mg, 10%) as a white solid. ESI-MS [M+H]⁺: 572.2. ¹H NMR (400 MHz, DMSO) δ 10.45 (s, 1H), 8.46 (d, J=5.1 Hz, 1H), 8.33 (s, 1H), 8.30-8.27 (m, 1H), 8.02-8.01 (m, 2H), 7.14 (d, J=5.1 Hz, 1H), 4.86 (s, 2H), 4.65 (d, J=5.5 Hz, 2H), 2.96 (s, 3H), 2.55-2.52 (m, 2H), 2.38 (s, 3H), 2.03-2.00 (m, 1H), 1.52-1.49 (m, 2H), 0.88-0.86 (m, 4H).

### Example 85

### Synthesis of (1S,2S)—N-(6-(((6-cyclopropyl-8-(2-oxopyrrolidin-1-yl)imidazo[1,2-a]pyrazin-2-yl)methyl)(methyl)amino)pyrimidin-4-yl)-2-(4-methylpyrimidin-2-yl)cyclopropane-1-carboxamide (I-85)

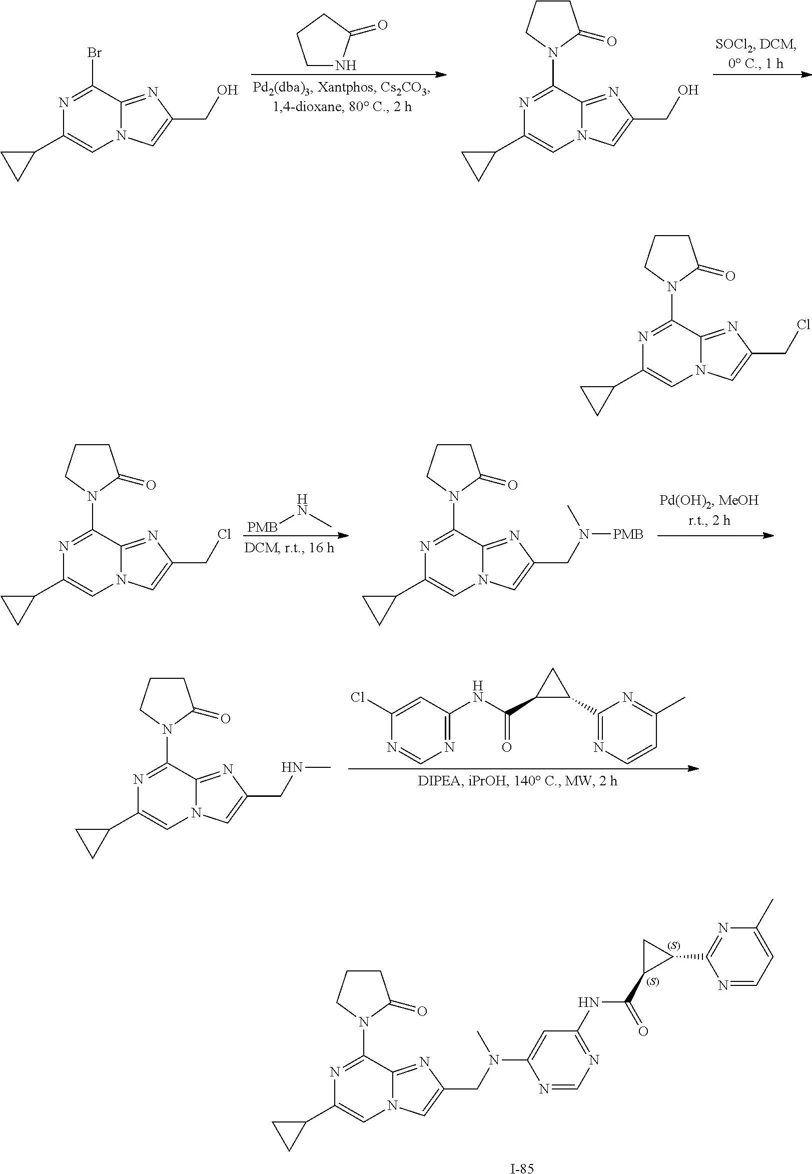

Synthesis of 1-(6-cyclopropyl-2-(hydroxymethyl)imidazo[1,2-a]pyrazin-8-yl)pyrrolidin-2-one. To a mixture of (8-bromo-6-cyclopropylimidazo[1,2-a]pyrazin-2-yl)methanol (400 mg, 1.5 mmol), pyrrolidin-2-one (510 mg, 6.0 mmol) and Cs₂CO₃ (1.5 g, 4.5 mmol) in 1,4-dioxane (40 mL) was added Pd₂(dba)₃ (274.5 mg, 0.30 mmol) and Xantphos (346.8 mg, 0.60 mmol). The reaction mixture was stirred at 80° C. for 2 h under N₂. The reaction mixture was cooled to room temperature, filtered through Celite® and the filter cake was washed with DCM/MeOH (10/1, 100 mL). The filtrate was concentrated in vacuo to give the crude, which was purified with preparative TLC (DCM/MeOH=10/1) to afford 1-(6-cyclopropyl-2-(hydroxymethyl)imidazo[1,2-a]pyrazin-8-yl)pyrrolidin-2-one (200 mg, 49%) as yellow oil. ESI-MS [M+H]⁺:273.1.

Synthesis of 1-(2-(chloromethyl)-6-cyclopropylimidazo[1,2-a]pyrazin-8-yl)pyrrolidin-2-one. A mixture of 1-(6-cyclopropyl-2-(hydroxymethyl)imidazo[1,2-a]pyrazin-8-yl)pyrrolidin-2-one (50 mg, 0.18 mmol) and SOCl₂ (0.2 mL) in DCM (5 mL) was stirred at 0° C. for 1 h. The reaction mixture was concentrated in vacuo to afford 1-(2-(chloromethyl)-6-cyclopropylimidazo[1,2-a]pyrazin-8-yl)pyrrolidin-2-one (50 mg, crude) as yellow oil. ESI-MS [M+H]⁺:291.1.

Synthesis of 1-(6-cyclopropyl-2-(((4-methoxybenzyl)(methyl)amino)methyl)imidazo[1,2-a]pyrazin-8-yl)pyrrolidin-2-one. To the mixture of 1-(2-(chloromethyl)-6-cyclopropylimidazo[1,2-a]pyrazin-8-yl)pyrrolidin-2-one (50 mg, crude) in DCM (5 mL) was added 1-(4-methoxyphenyl)-N-methylmethanamine (166.1 mg, 1.1 mmol). The reaction solution was stirred at room temperature for 16 h. The mixture was concentrated in vacuo and the residue was purified by column chromatography on silica gel eluting with a gradient of 0-10% MeOH in DCM to afford 1-(6-cyclopropyl-2-(((4-methoxybenz yl)(methyl)amino)methyl)imidazo[1,2-a]pyrazin-8-yl)pyrolidine-2-one (40 mg, 55% over 2 steps) as yellow oil. ESI-MS [M+H]⁺:406.2.

Synthesis of 1-(6-cyclopropyl-2-((methylamino)methyl)imidazo[1,2-a]pyrazin-8-yl) pyrrolidin-2-one. To a mixture of 1-(6-cyclopropyl-2-(((4-methoxybenzyl)(methyl)amino)methyl) imidazo[1,2-a]pyrazin-8-yl)pyrrolidin-2-one (40 mg, 0.1 mmol) in MeOH (3 mL) was added Pd(OH)₂ (20 mg). The reaction mixture was stirred for at room temperature for 2 h. The reaction mixture was filtered through Celite® and the filter cake was washed with MeOH (15 mL). The filtrate was concentrated in vacuo to give the crude, which was purified by preparative TLC (DCM/MeOH=10/1) to afford 1-(6-cyclopropyl-2-((methylamino)methyl)imidazo[1,2-a]pyrazin-8-yl)pyrrolidin-2-one (15 mg, 53% ) as yellow oil. ESI-MS [M+H]⁺:286.1.

Synthesis of (1S,2S)—N-(6-(((6-cyclopropyl-8-(2-oxopyrrolidin-1-yl)imidazo[1,2-a]pyrazin-2-yl)methyl)(meth yl)amino)pyrimidin-4-yl)-2-(4-methylpyrimidin-2-yl)cyclopropane-1-carboxamide. To a mixture of 1-(6-cyclopropyl-2-((methylamino)methyl)imidazo[1,2-a]pyrazin-8-yl)pyrrolidin-2-one (15 mg, 0.053 mmol), (1S,2S)—N-(6-chloropyrimidin-4-yl)-2-(4-methylpyrimidin-2-yl)cyclopropane-1-carboxamide (18 mg, 0.058 mmol) and DIPEA (21 mg, 0.16 mmol) in i-PrOH (2 mL) was stirred in a sealed tube. The reaction was irradiated in a microwave reactor at 140° C. for 2 h. The reaction was cooled to room temperature and concentrated in vacuo to give the crude, which was purified by preparative TLC (DCM/MeOH=10/1) to afford (1S,2S)—N-(6-(((6-cyclopropyl-8-(2-oxopyrrolidin-1-yl)imidazo[1,2-a]pyrazin-2-yl)methyl)(methyl)amino)pyrimidin-4-yl)-2-(4-methylpyrimidin-2-yl)cyclopropane-1-carboxamide (8.8 mg, 31%) as a yellow solid. ESI-MS [M+H]⁺:539.2. ¹H NMR (400 MHz, DMSO) δ 10.84 (s, 1H), 8.53 (d, J=5.1 Hz, 1H), 8.31-8.27 (m, 2H), 7.77 (s, 1H), 7.42 (s, 1H), 7.21 (d, J=5.1 Hz, 1H), 4.89 (s, 2H), 4.02 (t, J=7.0 Hz, 2H), 3.10 (s, 3H), 2.65-2.62 (m, 1H), 2.56-2.52 (m, 3H), 2.41 (s, 3H), 2.18-2.10 (m, 2H), 2.04-1.98 (m, 1H), 1.57-1.50 (m, 2H), 0.88-0.81 (m, 4H).

### Example 86

### Synthesis of (S,2S)—N-(6-(((5-cyclopropyl-7-(3-methyl-2,4-dioxoimidazolidin-1-yl)-1H-benzo[d]imidazol-2-yl)methyl)(methyl)amino)-2-methylpyrimidin-4-yl)-2-(4-methylpyrimidin-2-yl)cyclopropane-1-carboxamide (I-86)

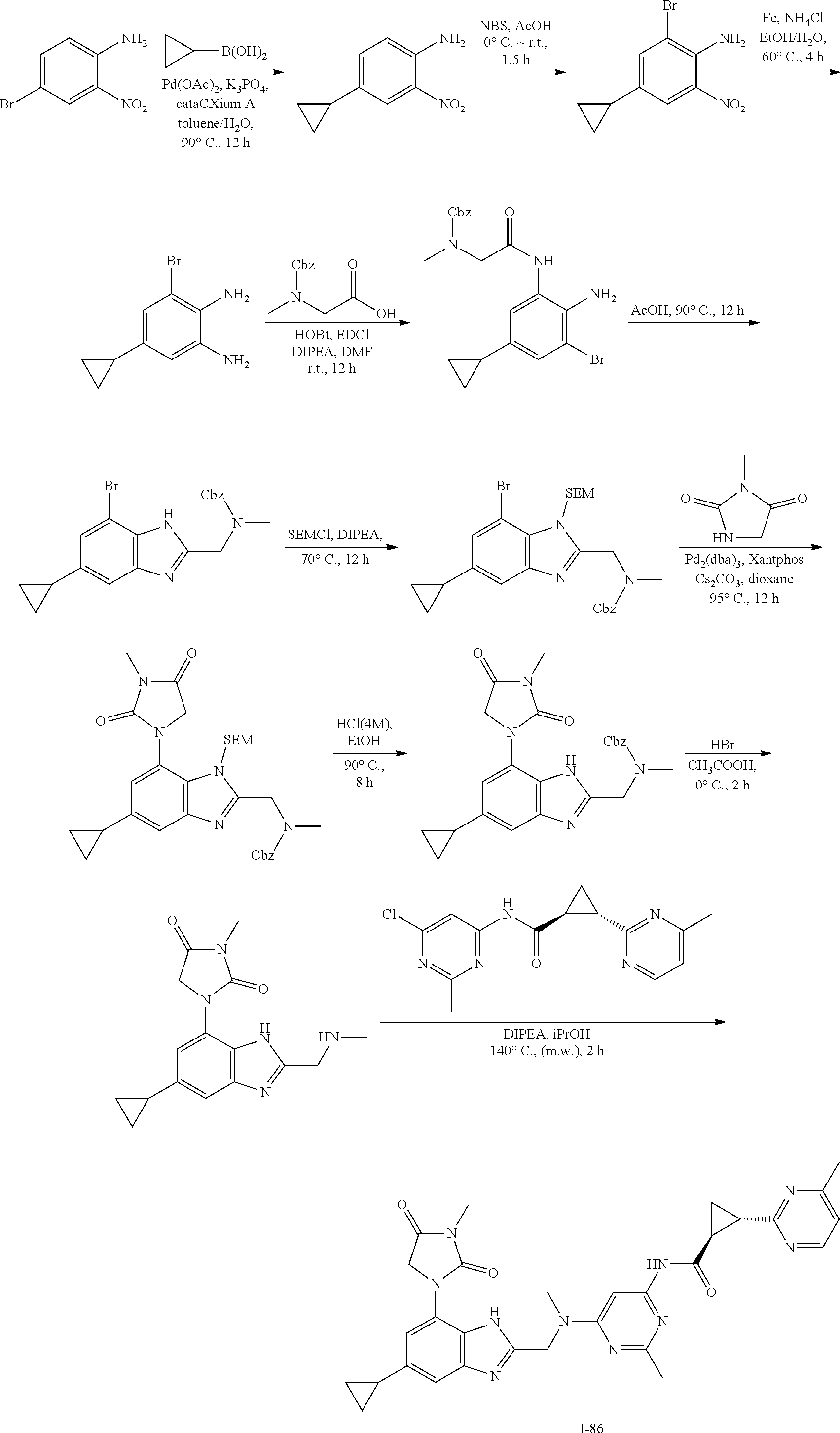

Synthesis of 4-cyclopropyl-2-nitroaniline. A solution of 4-bromo-2-nitroaniline (3 g, 13.9 mmol), cyclopropylboronic acid (1.8 g, 20.9 mmol), Pd(OAc)₂ (314 mg, 1.4 mmol), cataCXium A (501 mg, 1.4 mmol) and K₃PO₄ (8.8 g, 41.7 mmol) in toluene/H₂O (60 mL/6 mL) was stirred at 90° C. for 12 h. The reaction mixture was cooled to room temperature, filtered and the filtrate was concentrated in vacuo to give the crude, which was purified with silica gel chromatography eluting with a gradient of 0-7% MeOH in DCM to give 4-cyclopropyl-2-nitroaniline (2.4 g, 96%) as a yellow solid. ESI-MS [M+H]⁺: 179.2

Synthesis of 2-bromo-4-cyclopropyl-6-nitroaniline. To a solution of 4-cyclopropyl-2-nitroaniline (2.5 g, 14.0 mmol) in AcOH (50 mL) was added NBS (2.9 g, 16.8 mmol) at 0° C. for 1.5 h. After completed, the reaction was diluted with H₂O (80 mL), extracted with EtOAc (50 mL×3). The combined organic layers were washed with brine (60 mL), dried over anhydrous Na₂SO₄ and concentrated in vacuo to give the crude, which was purified with silica gel eluting with a gradient of 0-5% MeOH in DCM to give the 2-bromo-4-cyclopropyl-6-nitroaniline as a yellow solid. (2 g, 56%), ESI-MS [M+H]⁺: 257.2.

Synthesis of 3-bromo-5-cyclopropylbenzene-1,2-diamine. To a solution of 2-bromo-4-cyclopropyl-6-nitroaniline (2 g, 7.8 mmol), Fe (2.2 g, 39.0 mmol) and NH₄Cl (2.1 g, 39.0 mmol) in EtOH/H₂O (40 mL/4 mL) was stirred at 60° C. for 4 h. The reaction mixture was filtered through the Celite®, and the filter cake was washed with MeOH (50 mL). The filtrate was concentrated in vacuo to give the crude, which was purified with silica gel column chromatography eluting with a gradient of 1-5% MeOH in DCM to give the 3-bromo-5-cyclopropylbenzene-1,2-diamine as a yellow solid. (800 mg, 45%), ESI-MS [M+H]⁺: 227.2

Synthesis of benzyl (2-((2-amino-3-bromo-5-cyclopropylphenyl)amino)-2-oxoethyl)(methyl)carbamate. To a solution of 3-bromo-5-cyclopropylbenzene-1,2-diamine (500 mg, 2.2 mmol), N-((benzyloxy)carbonyl)-N-methylglycine (736 mg, 3.3 mmol), EDCI (637 mg, 3.3 mmol), HOBt (446 mg, 3.3 mmol) and DIPEA (851 mg, 6.6 mmol) in DMF (20 mL) was stirred at room temperature for 12 h. The reaction was quenched with H₂O (40 mL), extracted with EtOAc (40 mL×3). The combined organic layers were washed with brine (50 mL), dried over Na₂SO₄ then concentrated in vacuo to give the benzyl (2-((2-amino-3-bromo-5-cyclopropylphenyl)amino)-2-oxoethyl)(methyl)carbamate (700 mg, crude) as a white solid. ESI-MS [M+H]⁺: 432.2.

Synthesis of benzyl ((7-bromo-5-cyclopropyl-1H-benzo[d]imidazol-2-yl)methyl)(methyl)carbamate. A solution of benzyl (2-((2-amino-3-bromo-5-cyclopropylphenyl)amino)-2-oxoethyl)(methyl)carbamate (700 mg, crude) in AcOH (10 mL) was stirred at 90° C. for 12 h. The reaction was quenched with NaHCO₃ (sat. aq., 80 mL) then extracted with EtOAc (80 mL×3). The combined organic layers were washed with brine (60 mL), dried over anhydrous Na₂SO₄, concentrated in vacuo and purified by silica gel column chromatography eluting with a gradient of 0-8% MeOH in DCM to give benzyl ((7-bromo-5-cyclopropyl-1H-benzo[d]imidazol-2-yl)methyl)(methyl)carbamate as a yellow solid. (250 mg, 27% over 2 steps). ESI-MS [M+H]⁺: 414.2.

Synthesis of benzyl ((7-bromo-5-cyclopropyl-1-((2-(trimethylsilyl)ethoxy)methyl)-1H-benzo[d]imidazol-2-yl)methyl)(methyl)carbamate. To a solution of benzyl ((7-bromo-5-cyclopropyl-1H-benzo[d]imidazol-2-yl)methyl)(methyl)carbamate (100 mg, 0.24 mmol) and DIPEA (93 mg, 0.72 mmol ) in THF (20 mL) was added SEMCl (120 mg, 0.72 mmol ) at room temperature under nitrogen. The reaction mixture was stirred at 70° C. for 12 h. The reaction was cooled to room temperature, quenched with H₂O (30 mL) and extracted with EtOAc (30 mL×3). The combined organic layers were washed with brine (40 mL), dried over anhydrous Na₂SO₄ then concentrated in vacuo to give the crude, which was purified with silica gel column chromatography eluting with a gradient of 0-30% EtOAc in PE to give benzyl ((7-bromo-5-cyclopropyl-1-((2-(trimethylsilyl)ethoxy)methyl)-1H-benzo[d]imidazol-2-yl)methyl)(methyl)carbamate (60 mg, 46%) as a yellow solid. ESI-MS [M+H]⁺: 544.2

Synthesis of benzyl ((5-cyclopropyl-7-(3-methyl-2,4-dioxoimidazolidin-1-yl)-1-((2-(trimethylsilyl)ethoxy)methyl)-1H-benzo[d]imidazol-2-yl)methyl)(methyl)carbamate. To a mixture of 1-(7-bromo-5-cyclopropyl-1-((2-(trimethylsilyl)ethoxy)methyl)-1H-benzo[d]imidazol-2-yl)-N-methylmethanamine (60 mg, 0.11 mmol), 3-methylimidazolidine-2,4-dione (23 mg, 0.20 mmol) and Cs₂CO₃ (108 mg, 0.33 mmol) in 1,4-dioxane (10 mL) was added Pd₂(dba)₃ (10 mg, 0.011 mmol) and Xantphos (6 mg, 0.011 mmol). The reaction mixture was stirred at 95° C. for 12 h under nitrogen. The reaction mixture was cooled to room temperature and filtered through Celite®. The filter cake was washed with DCM/MeOH (10/1, 20 mL). The filtrate was concentrated in vacuo to give the crude, which was purified by silica gel column chromatography eluting with a gradient of 0-5% MeOH in DCM to give benzyl ((5-cyclopropyl-7-(3-methyl-2,4-dioxoimidazolidin-1-yl)-1-((2-(trimethylsilyl)ethoxy)methyl)-1H-benzo[d]imidazol-2-yl)methyl)(methyl)carbamate (40 mg, 63%) as a yellow solid. ESI-MS [M+H]⁺: 578.2.

Synthesis of benzyl ((5-cyclopropyl-7-(3-methyl-2,4-dioxoimidazolidin-1-yl)-1H-benzo[d]imidazol-2-yl)methyl)(methyl)carbamate. To a solution of benzyl ((5-cyclopropyl-7-(3-methyl-2,4-dioxoimidazolidin-1-yl)-1-((2-(trimethylsilyl)ethoxy)methyl)-1H-benzo[d]imidazol-2-yl)methyl)(methyl)carbamate (40 mg, 0.07 mmol) in EtOH (4 mL) was added HCl (4M solution in 1,4-dioxane, 1 mL). The resulting mixture was stirred at 90° C. for 8 h. The reaction mixture was concentrated in vacuo to give benzyl ((5-cyclopropyl-7-(3-methyl-2,4-dioxoimidazolidin-1-yl)-1H-benzo[d]imidazol-2-yl)methyl)(methyl)carbamate as the hydrochloric acid salt (35 mg, crude) as a yellow solid. ESI-MS [M+H]⁺: 448.2.

Synthesis of 1-(5-cyclopropyl-2-((methylamino)methyl)-1H-benzo[d]imidazol-7-yl)-3-methylimidazolidine-2,4-dione. A solution of benzyl ((5-cyclopropyl-7-(3-methyl-2,4-dioxoimidazolidin-1-yl)-1H-benzo[d]imidazol-2-yl)methyl)(methyl)carbamate (35 mg, crude) and HBr (33% solution in AcOH, 2 mL) in AcOH (3 mL) was stirred at 0° C. for 2 h. The reaction was quenched with NaHCO₃ (sat. aq., 30 mL) and extracted with EtOAc (30 mL×3). The combined organic layers were washed with brine (30 mL), dried over anhydrous Na₂SO₄ then concentrated in vacuo to give the crude, which was purified with silica gel column chromatography eluting with a gradient of 0-8% MeOH in DCM to give 1-(5-cyclopropyl-2-((methylamino)methyl)-1H-benzo[d]imidazol-7-yl)-3-methylimidazolidine-2,4-dione (20 mg, 91% for 2 steps) as a yellow solid. ESI-MS [M+H]⁺: 314.2.

Synthesis of (1S,2S)—N-(6-(((5-cyclopropyl-7-(3-methyl-2,4-dioxoimidazolidin-1-yl)-1H-benzo[d]imidazol-2-yl)methyl)(methyl)amino)-2-methylpyrimidin-4-yl)-2-(4-methylpyrimidin-2-yl)cyclopropane-1-carboxamide. A solution of 1-(5-cyclopropyl-2-((methylamino)methyl)-1H-benzo[d]imidazol-7-yl)-3-methylimidazolidine-2,4-dione (20 mg, 0.06 mmol), (1S,2S)—N-(6-chloro-2-methylpyrimidin-4-yl)-2-(4-methylpyrimidin-2-yl)cyclopropane-1-carboxamide (30 mg, 0.10 mmol) and DIPEA (19 mg, 0.15 mmol) in i-PrOH (2 mL) was stirred in a sealed tube. The reaction was irradiated in a microwave reactor at 140° C. for 2 h. The reaction was cooled to room temperature then concentrated in vacuo to give the crude product, which was purified by preparative TLC (eluent: DCM/MeOH=10/1) to give the (1S,2S)—N-(6-(((5-cyclopropyl-7-(3-methyl-2,4-dioxoimidazolidin-1-yl)-1H-benzo[d]imidazol-2-yl)methyl)(methyl)amino)-2-methylpyrimidin-4-yl)-2-(4-methylpyrimidin-2-yl)cyclopropane-1-carboxamide (20 mg, 54%) as a white solid. ESI-MS [M+H]⁺: 581.2. ESI-MS [M+H]⁺: 581.3. ¹H NMR (400 MHz, DMSO) δ 12.23 (s, 1H), 10.83 (s, 1H), 8.52 (d, J=5.1 Hz, 1H), 7.35-7.24 (m, 2H), 7.20 (d, J=5.2 Hz, 1H), 6.99-6.91 (m, 1H), 4.99 (s, 2H), 4.90 (d, J=1.6 Hz, 2H), 3.12-3.08 (m, 3H), 2.96 (s, 3H), 2.64-2.61 (m, 2H), 2.41 (s, 3H), 2.30 (s, 3H), 2.04-1.94 (m, 1H), 1.57-1.43 (m, 2H), 1.00-0.90 (m, 2H), 0.70-0.57 (m, 2H).

### Example 87

### Synthesis of (1S,2S)—N-(4-((6-cyclopropyl-8-(3-methyl-2,4-dioxoimidazolidin-1-yl)imidazo[1,2-a]pyrazin-2-yl)methoxy)-6-methyl-1,3,5-triazin-2-yl)-2-(4-methylpyrimidin-2-yl)cyclopropane-1-carboxamide (I-87)

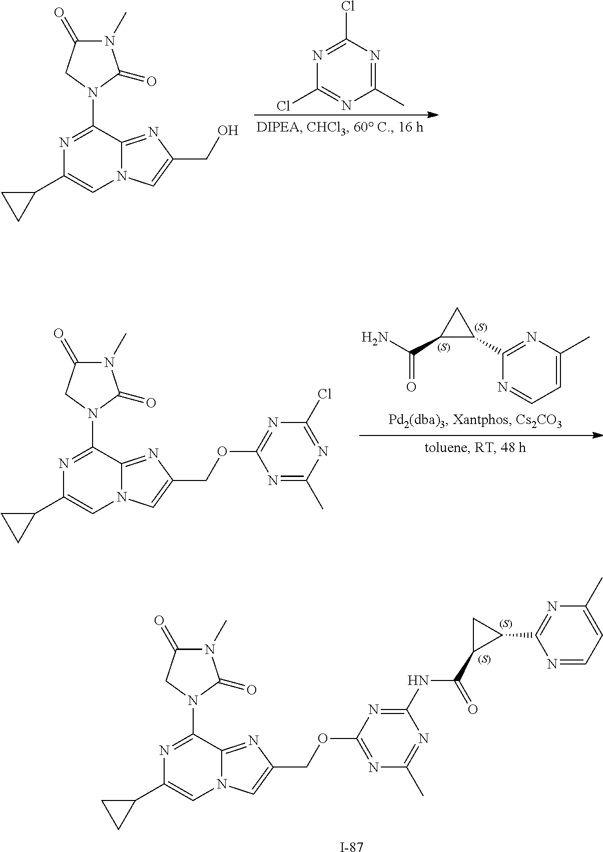

Synthesis of 1-(2-(((4-chloro-6-methyl-1,3,5-triazin-2-yl)oxy)methyl)-6-cyclopropylimidazo[1,2-a]pyrazin-8-yl)-3-methylimidazolidine-2,4-dione. A mixture of 1-(6-cyclopropyl-2-(hydroxymethyl)imidazo[1,2-a]pyrazin-8-yl)-3-methylimidazolidine-2,4-dione (200 mg, 0.66 mmol), 2,4-dichloro-6-methyl-1,3,5-triazine (538 mg, 3.3 mmol) and DIPEA (426 mg, 3.3 mmol) in CHCl₃ (10 mL) was stirred at 60° C. for 16 h. The reaction mixture was cooled to room temperature. Water (50 mL) was added and the mixture was extracted with DCM (30 mL×3). The combined organic layers were washed with brine (30 mL), dried over anhydrous Na₂SO₄, and concentrated in vacuo to give the crude, which was purified by column chromatography (eluent: DCM/MeOH=30/1) to give 1-(2-(((4-chloro-6-methyl-1,3,5-triazin-2-yl)oxy)methyl)-6-cyclopropylimidazo[1,2-a]pyrazin-8-yl)-3-methylimidazolidine-2,4-dione (176 mg, 62%) as a yellow solid. ESI-MS [M+H]⁺: 429.2.

Synthesis of (1S,2S)—N-(4-((6-cyclopropyl-8-(3-methyl-2,4-dioxoimidazolidin-1-yl)imidazo[1,2-a]pyrazin-2-yl)methoxy)-6-methyl-1,3,5-triazin-2-yl)-2-(4-methylpyrimidin-2-yl)cyclopropane-1-carboxamide. A solution of 1-(2-(((4-chloro-6-methyl-1,3,5-triazin-2-yl)oxy)methyl)-6-cyclopropylimidazo[1,2-a]pyrazin-8-yl)-3-methylimidazolidine-2,4-dione (80 mg, 0.19 mmol), (1S,2S)-2-(4-methylpyrimidin-2-yl)cyclopropane-1-carboxamide (50 mg, 0.28 mmol), XantPhos (35 mg, 0.06 mmol), Pd₂(dba)₃ (27 mg, 0.03 mmol) and Cs₂CO₃ (124 mg, 0.38 mmol) in toluene (5 mL) was stirred at room temperature in for 48 h under N₂ atmosphere. The mixture was concentrated in vacuo. The residue was diluted with H₂O (50 mL) and extracted with EtOAc (30 mL×3). The combined organics were washed with brine (30 mL), dried over Na₂SO₄, and concentrated in vacuo. The residue was purified by preparative TLC, eluting with 5% MeOH in DCM to give (1S,2S)—N-(4-((6-cyclopropyl-8-(3-methyl-2,4-dioxoimidazolidin-1-yl)imidazo[1,2-a]pyrazin-2-yl)methoxy)-6-methyl-1,3,5-triazin-2-yl)-2-(4-methylpyrimidin-2-yl)cyclopropane-1-carboxamide (18 mg, yield 17%) as a white solid. ESI-MS [M+H]⁺: 570.2, ¹H NMR (400 MHz, DMSO) δ 11.24 (s, 1H), 8.50 (d, J=5.1 Hz, 1H), 8.39 (s, 1H), 8.16 (s, 1H), 7.17 (d, J=5.1 Hz, 1H), 5.48 (s, 2H), 4.87 (s, 2H), 3.03-2.91 (m, 4H), 2.61-2.57 (m, 1H), 2.40-2.36 (m, 6H), 2.09-2.00 (m, 1H), 1.63-1.55 (m, 2H), 0.93-0.85 (m, 4H).

### Example 88

### Synthesis of (S,2S)—N-(6-(((6-(6,6-difluoro-3-azabicyclo[3.1.0]hexan-3-yl)-2-methylpyridin-3-yl)methyl)amino)-2-methylpyrimidin-4-yl)-2-(4-methylpyrimidin-2-yl)cyclopropane-1-carboxamide (I-88)

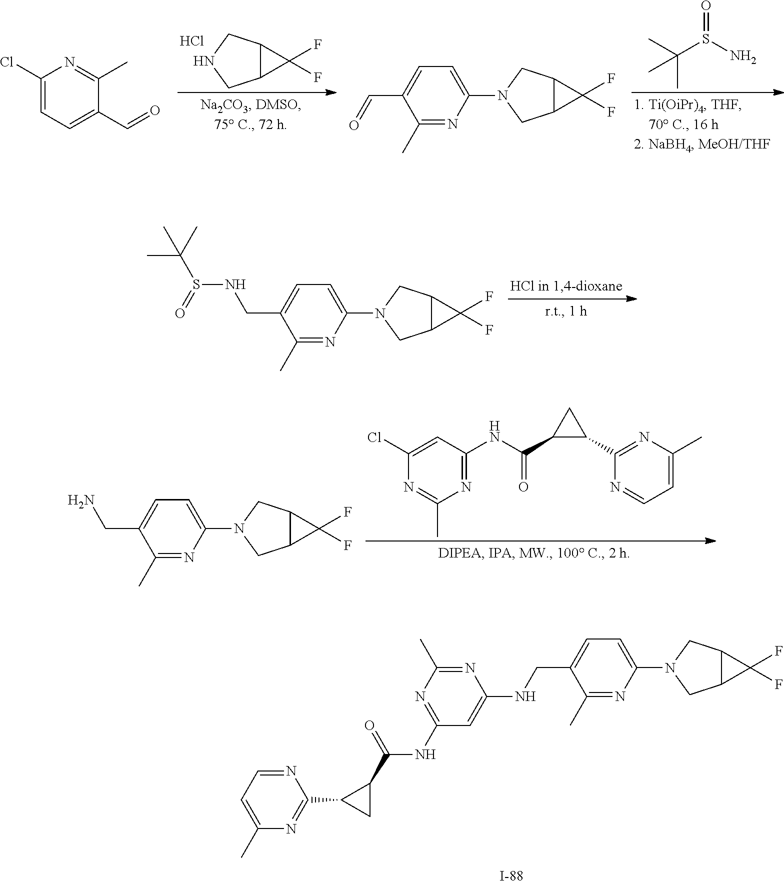

Synthesis of 6-(6,6-difluoro-3-azabicyclo[3.1.0]hexan-3-yl)-2-methylnicotinaldehyde. A mixture of 6-chloro-2-methylnicotinaldehyde (500 mg, 3.23 mmol), 6,6-difluoro-3-azabicyclo[3.1.0]hexane hydrochloride (500 mg, 3.23 mmol) and Na₂CO₃ (1.0 g, 9.69 mmol) in DMSO (10.0 mL) was stirred at 75° C. for 72 h. After cooling to room temperature, the reaction was quenched with water (100 mL) and extracted with EtOAc (30 mL×3). The combined organic layers were washed with brine (50 mL), dried over anhydrous Na₂SO₄, and concentrated in vacuo to give 6-(6,6-difluoro-3-azabicyclo[3.1.0]hexan-3-yl)-2-methylnicotinaldehyde (700 mg, 91%) as a yellow solid. ESI-MS [M+H]⁺: 239.2.

Synthesis of N-((6-(6,6-difluoro-3-azabicyclo[3.1.0]hexan-3-yl)-2-methylpyridin-3-yl)methyl)-2-methylpropane-2-sulfinamide. A mixture of 6-(6,6-difluoro-3-azabicyclo[3.1.0]hexan-3-yl)-2-methylnicotinaldehyde (300 mg, 1.26 mmol), 2-methylpropane-2-sulfinamide (229 mg, 1.89 mmol) and Ti(O-iPr)₄ (1.8 g, 6.3 mmol) in THF (10.0 mL) was stirred at 70° C. for 16 h. The reaction mixture was cooled to 0° C. then a mixture of NaBH₄ (144 mg, 3.78 mmol) and MeOH (5.0 mL) was added. The mixture was stirred at room temperature for 1 h. The reaction was quenched with water (30 mL) and extracted with EtOAc (20 mL×3). The combined organic layers were washed with brine (20 mL), dried over anhydrous Na₂SO₄, and concentrated in vacuo to give the crude product, which was purified by preparative TLC (eluent: DCM/MeOH=20/1) to give N-((6-(6,6-difluoro-3-azabicyclo[3.1.0]hexan-3-yl)-2-methylpyridin-3-yl)methyl)-2-methylpropane-2-sulfinamide (350 mg, 81%) as alight yellow solid. ESI-MS [M+H]⁺: 344.2.

Synthesis of (6-(6,6-difluoro-3-azabicyclo[3.1.0]hexan-3-yl)-2-methylpyridin-3-yl)methanamine. A mixture of N-((6-(6,6-difluoro-3-azabicyclo[3.1.0]hexan-3-yl)-2-methylpyridin-3-yl)methyl)-2-methylpropane-2-sulfinamide (350 mg, 1.02 mmol) in HCl (4M solution in 1,4-dioxane, 5 mL) was stirred at room temperature for 1 h. The reaction was quenched with NH₃ (7M solution in MeOH, 5 mL) and concentrated in vacuo to give the crude, which was purified by preparative TLC (eluent: DCM/MeOH=10/1) to give the product (6-(6,6-difluoro-3-azabicyclo[3.1.0]hexan-3-yl)-2-methylpyridin-3-yl)methanamine (100 mg, 41%) as a white solid. ESI-MS [M+H]⁺: 240.2.

Synthesis of (1S,2S)—N-(6-(((6-(6,6-difluoro-3-azabicyclo[3.1.0]hexan-3-yl)-2-methylpyridin-3-yl)methyl)amino)-2-methylpyrimidin-4-yl)-2-(4-methylpyrimidin-2-yl) cyclopropane-1-carboxamide. A mixture of (6-(6,6-difluoro-3-azabicyclo[3.1.0]hexan-3-yl)-2-methylpyridin-3-yl)methanamine (48 mg, 0.2 mmol), (1S,2S)—N-(6-chloro-2-methylpyrimidin-4-yl)-2-(4-methylpyrimidin-2-yl)cyclopropane-1-carboxamide (61 mg, 0.2 mmol) and DIPEA (129 mg, 1.0 mmol) in i-PrOH (5 mL) was stirred in a sealed tube. After degassing with N₂ for 1 min, the reaction was irradiated in a microwave reactor at 100° C. for 2 h. After cooling to room temperature, the mixture was quenched with water (30 mL) and extracted with EtOAc (20 mL×3). The combined organic layers were washed with brine (20 mL), dried over Na₂SO₄ and concentrated in vacuo. The residue was purified by preparative HPLC to give (1S,2S)—N-(6-(((6-(6,6-difluoro-3-azabicyclo[3.1.0]hexan-3-yl)-2-methylpyridin-3-yl)methyl)amino)-2-methylpyrimidin-4-yl)-2-(4-methylpyrimidin-2-yl)cyclopropane-1-carboxamide (20 mg, 20%) as a white solid. ESI-MS [M+H]⁺: 507.2. ¹H NMR (400 MHz, DMSO) δ 10.64 (s, 1H), 8.52 (d, J=3.8 Hz, 1H), 7.56 (s, 1H), 7.37 (d, J=7.7 Hz, 1H), 7.20 (d, J=4.8 Hz, 1H), 7.04 (s, 1H), 6.24 (d, J=8.4 Hz, 1H), 4.31 (s, 2H), 3.74 (d, J=10.7 Hz, 2H), 3.61 (d, J=10.4 Hz, 2H), 2.71-2.53 (m, 4H), 2.41 (s, 3H), 2.36 (s, 3H), 2.26 (s, 3H), 1.52-1.47 (m, 2H).

### Example 89

### Synthesis of (1S,2S)—N-(6-((1-(6-cyclopropyl-8-(3-methyl-2,4-dioxoimidazolidin-1-yl)imidazo[1,2-b]pyridazin-2-yl)ethyl)amino)-2-methylpyrimidin-4-yl)-2-(4-methylpyrimidin-2-yl)cyclopropane-1-carboxamide (I-89)

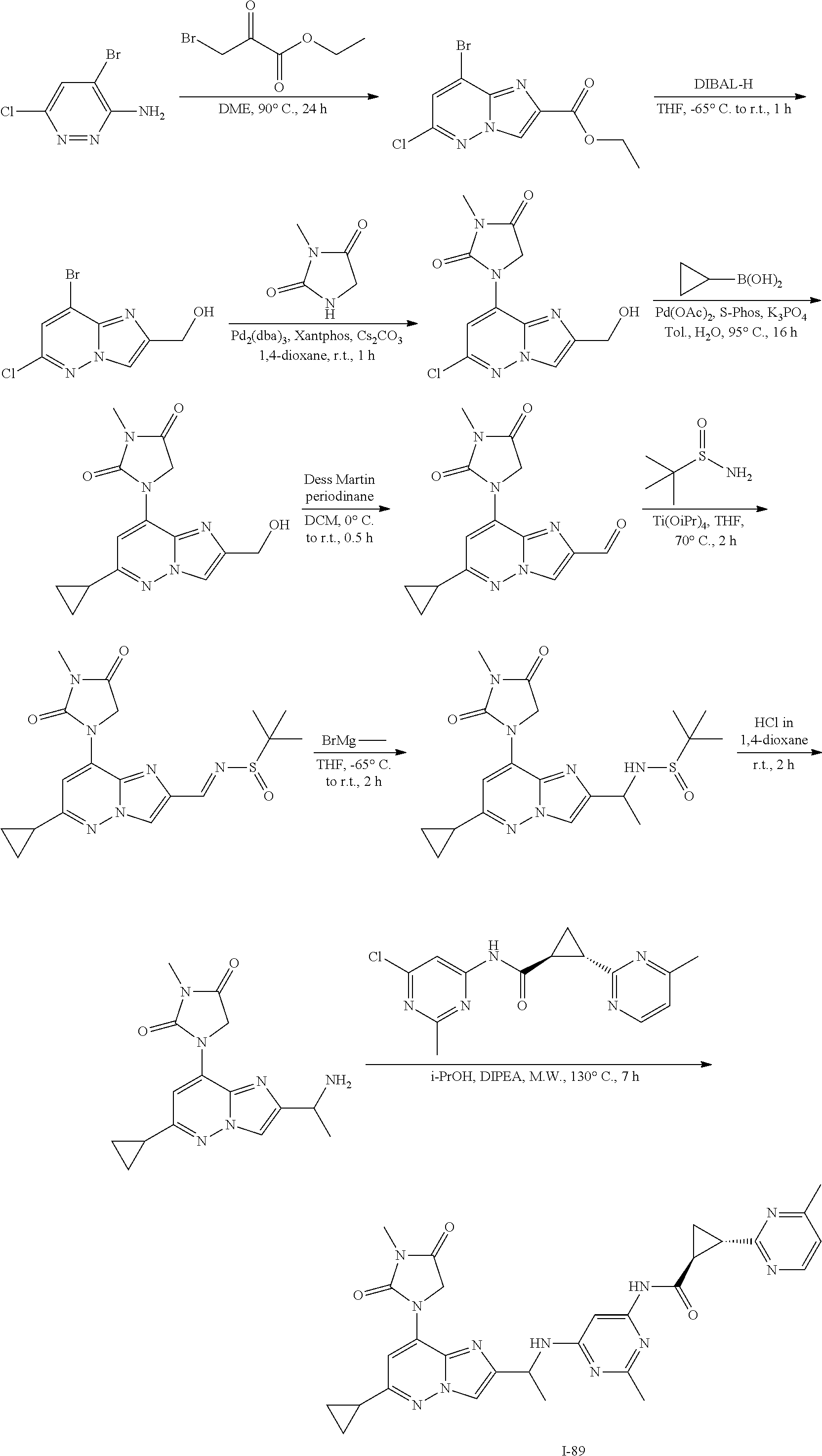

Synthesis of ethyl 8-bromo-6-chloroimidazo[1,2-b]pyridazine-2-carboxylate. A mixture of 4-bromo-6-chloropyridazin-3-amine (10 g, 47.8 mmol) and ethyl 3-bromo-2-oxopropanoate (13.9 g, 71.7 mmol) in DME (200 mL) was stirred at 90° C. for 24 h under N₂. After cooling to room temperature, the reaction mixture was concentrated in vacuo. Water (300 mL) was added and the mixture was extracted with EtOAc (200×3 mL). The combined organic layers were washed with brine (300 mL), dried over anhydrous Na₂SO₄, and concentrated in vacuo to give the crude product, which was purified by column chromatography (eluent: PE/EtOAc=0˜20%) to give ethyl 8-bromo-6-chloroimidazo[1,2-b]pyridazine-2-carboxylate (7.9 g, 55%) as a white solid. ESI-MS [M+H]⁺: 304.1.

Synthesis of (8-bromo-6-chloroimidazo[1,2-b]pyridazin-2-yl)methanol. To a mixture of ethyl 8-bromo-6-chloroimidazo[1,2-b]pyridazine-2-carboxylate (5.0 g, 16.5 mmol) in THF (50 mL) was added DIBAL-H (1 M solution in hexanes, 49.5 mL, 49.5 mmol) at −65° C. under N₂. The mixture was stirred at −65° C. for 1 h and warmed to room temperature. The mixture was stirred at room temperature for 1 h. The reaction was quenched with water (80 mL) and extracted with EtOAc (50 mL×3). The combined organic layers were washed with brine (80 mL), dried over anhydrous Na₂SO₄, and concentrated in vacuo to give the crude, which was purified by column chromatography (eluent: PE/EtOAc=0˜35%) to give (8-bromo-6-chloroimidazo[1,2-b]pyridazin-2-yl)methanol (2.3 g, 53%) as a white solid. ESI-MS [M+H]⁺: 262.1.

Synthesis of 1-(6-chloro-2-(hydroxymethyl)imidazo[1,2-b]pyridazin-8-yl)-3-methylimidazolidine-2,4-dione. To a mixture of (8-bromo-6-chloroimidazo[1,2-b]pyridazin-2-yl)methanol (2.3 g, 8.8 mmol), 3-methylimidazolidine-2,4-dione (3.0 g, 26.4 mmol) and Cs₂CO₃ (7.2 g, 22.0 mmol) in 1,4-dioxane (200 mL) was added Pd₂(dba)₃ (806 mg, 0.88 mmol) and Xantphos (1.0 g, 1.76 mmol). The mixture was stirred at room temperature for 1 h under N₂. The reaction mixture was cooled to room temperature. The mixture was filtered through Celite® and the filter cake was washed with DCM/MeOH (10/1, 50 mL). The filtrate was concentrated in vacuo to give the crude, which was purified by column chromatography (eluent: PE/EtOAc=0˜30%) to give 1-(6-chloro-2-(hydroxymethyl)imidazo[1,2-b]pyridazin-8-yl)-3-methylimidazolidine-2,4-dione (1.5 g, 58%) as a white solid. ESI-MS [M+H]⁺: 296.2.

Synthesis of 1-(6-cyclopropyl-2-(hydroxymethyl)imidazo[1,2-b]pyridazin-8-yl)-3-methylimidazolidine-2,4-dione . To a mixture of 1-(6-chloro-2-(hydroxymethyl)imidazo[1,2-b]pyridazin-8-yl)-3-methylimidazolidine-2,4-dione (1.48 g, 5.0 mmol), cyclopropylboronic acid (860 mg, 10.0 mmol) and K₃PO₄ (3.2 g, 15.0 mmol) in toluene (100 mL) and H₂O (10 mL) was added Pd(OAc)₂ (112 mg, 0.50 mmol) and S-Phos (410 mg, 1.0 mmol). The mixture was stirred at 95° C. for 16 h under N₂. The reaction mixture was cooled to room temperature. The mixture was filtered through Celite® and the filter cake was washed with DCM/MeOH (10/1, 50 mL). The filtrate was concentrated in vacuo to give the crude, which was purified by column chromatography (eluent: DCM/MeOH=0˜5%) to give 1-(6-cyclopropyl-2-(hydroxymethyl)imidazo[1,2-b]pyridazin-8-yl)-3-methylimidazolidine-2,4-dione (660 mg, 44%) as a white solid. ESI-MS [M+H]⁺: 302.2.

Synthesis of 6-cyclopropyl-8-(3-methyl-2,4-dioxoimidazolidin-1-yl)imidazo[1,2-b]pyridazine-2-carbaldehyde. To a mixture of 1-(6-cyclopropyl-2-(hydroxymethyl)imidazo[1,2-b]pyridazin-8-yl)-3-methylimidazolidine-2,4-dione (620 mg, 2.1 mmol) in DCM (30 mL) was added DMP (1.78 g, 4.2 mmol) at 0° C. The mixture was stirred at room temperature for 0.5 h. The reaction was quenched with NaHCO₃ (sat. aq., 20 mL) and Na₂S₂O₃ (sat. aq., 20 mL). The mixture was extracted with DCM (30 mL×3). The combined organic layers were washed with brine (50 mL), dried over anhydrous Na₂SO₄, and concentrated in vacuo to give the crude, which was purified by column chromatography preparative TLC (eluent: PE/EtOAc=0˜25%) to give 6-cyclopropyl-8-(3-methyl-2,4-dioxoimidazolidin-1-yl)imidazo[1,2-b]pyridazine-2-carbaldehyde (400 mg, 64%) as a white solid. ESI-MS [M+H]⁺: 300.2.

Synthesis of (E)-N-((6-cyclopropyl-8-(3-methyl-2,4-dioxoimidazolidin-1-yl)imidazo[1,2-b]pyridazin-2-yl)methylene)-2-methylpropane-2-sulfinamide. To a mixture of 6-cyclopropyl-8-(3-methyl-2,4-dioxoimidazolidin-1-yl)imidazo[1,2-b]pyridazine-2-carbaldehyde (380 mg, 1.3 mmol) and 2-methylpropane-2-sulfinamide (315 mg, 2.6 mmol) in THF (20 mL) was added Ti(i-PrO)₄ (1.85 g, 6.5 mmol). The mixture was stirred at 70° C. for 2 h. After cooling to room temperature, H₂O (50 mL) was added and the mixture was extracted with EtOAc (30 mL×3). The combined organic layers were washed with brine (50 mL), dried over anhydrous Na₂SO₄, and concentrated in vacuo to give the crude, which was purified by (eluent: PE/EtOAc=0˜30%) to give (E)-N-((6-cyclopropyl-8-(3-methyl-2,4-dioxoimidazolidin-1-yl)imidazo[1,2-b]pyridazin-2-yl)methylene)-2-methylpropane-2-sulfinamide (390 mg, 75%) as a white solid. ESI-MS [M+H]⁺: 403.2.

Synthesis of N-(1-(6-cyclopropyl-8-(3-methyl-2,4-dioxoimidazolidin-1-yl)imidazo[1,2-b]pyridazin-2-yl)ethyl)-2-methylpropane-2-sulfinamide. To a mixture of (E)-N-((6-cyclopropyl-8-(3-methyl-2,4-dioxoimidazolidin-1-yl)imidazo[1,2-b]pyridazin-2-yl)methylene)-2-methylpropane-2-sulfinamide (390 mg, 0.97 mmol) in THF (10 mL) was added methylmagnesium bromide (3M solution in diethyl ether, 1.6 mL, 4.9 mmol) dropwise at −65° C. under N₂. The mixture was stirred at room temperature for 2 h. The reaction was quenched with NH₄Cl (sat. aq., 30 mL) and extracted with EtOAc (20 mL×3). The combined organic layers were dried over Na₂SO₄, filtered and concentrated in vacuo to give the crude, which was purified by column chromatography (eluent: PE/EtOAc=0-30%) to give N-(1-(6-cyclopropyl-8-(3-methyl-2,4-dioxoimidazolidin-1-yl)imidazo[1,2-b]pyridazin-2-yl)ethyl)-2-methylpropane-2-sulfinamide (22 mg, 5%) as a white solid. ESI-MS [M+H]⁺: 419.2.

Synthesis of 1-(2-(1-aminoethyl)-6-cyclopropylimidazo[1,2-b]pyridazin-8-yl)-3-methylimidazolidine-2,4-dione. To a mixture of N-(1-(6-cyclopropyl-8-(3-methyl-2,4-dioxoimidazolidin-1-yl)imidazo[1,2-b]pyridazin-2-yl)ethyl)-2-methylpropane-2-sulfinamide (22 mg, 0.05 mmol) in 1,4-dioxane (1.0 mL) was added HCl (4 M solution in 1,4-dioxane, 1.0 mL) at 0° C. The reaction was stirred at room temperature for 1 h. The reaction was quenched with NH₃ (7 M solution in MeOH, 1 mL) and concentrated in vacuo to give the crude, which was purified by preparative TLC (eluent: DCM/MeOH=10/1) to give 1-(2-(1-aminoethyl)-6-cyclopropylimidazo[1,2-b]pyridazin-8-yl)-3-methylimidazolidine-2,4-dione (10 mg, 64%) as a white solid. ESI-MS [M+H]⁺: 315.2.

Synthesis of (1S,2S)—N-(6-((1-(6-cyclopropyl-8-(3-methyl-2,4-dioxoimidazolidin-1-yl)imidazo[1,2-b]pyridazin-2-yl)ethyl)amino)-2-methylpyrimidin-4-yl)-2-(4-methylpyrimidin-2-yl)cyclopropane-1-carboxamide. A mixture of 1-(2-(1-aminoethyl)-6-cyclopropylimidazo[1,2-b]pyridazin-8-yl)-3-methylimidazolidine-2,4-dione (10 mg, 0.032 mmol), (1S,2S)—N-(6-chloro-2-methylpyrimidin-4-yl)-2-(4-methylpyrimidin-2-yl)cyclopropane-1-carboxamide (11 mg, 0.035 mmol) and DIPEA (21 mg, 0.16 mmol) in i-PrOH (2 mL) was stirred in a sealed tube. After degassing with N₂ for 1 min, the reaction was irradiated in a microwave reactor at 130° C. for 7 h. The reaction was concentrated in vacuo to give the crude product, which was purified by preparative TLC (eluent: DCM/MeOH=10/1) to give (1S,2S)—N-(6-((1-(6-cyclopropyl-8-(3-methyl-2,4-dioxoimidazolidin-1-yl)imidazo[1,2-b]pyridazin-2-yl)ethyl)amino)-2-methylpyrimidin-4-yl)-2-(4-methylpyrimidin-2-yl)cyclopropane-1-carboxamide (7 mg, 38%) as a white solid. ESI-MS [M+H]⁺: 582.2. ¹H NMR (400 MHz, DMSO) δ 10.66 (s, 1H), 8.52 (d, J=4.8 Hz, 1H), 7.89 (s, 1H), 7.69 (s, 1H), 7.62 (s, 1H), 7.20 (d, J=4.4 Hz, 1H), 7.14 (s, 1H), 5.32-5.30 (m, 1H), 5.13 (s, 2H), 2.98 (s, 3H), 2.63-2.55 (m, 2H), 2.41 (s, 3H), 2.26 (s, 3H), 2.01-1.97 (m, 1H), 1.56-1.44 (m, 5H), 1.08-1.01 (m, 2H), 0.94-0.88 (m, 2H).

### Example 90

### Synthesis of (1S,2S)—N-(6-(((6-cyclopropylimidazo[1,2-a]pyrimidin-2-yl)methyl)(methyl)amino)-2-methylpyrimidin-4-yl)-2-(4-methylpyrimidin-2-yl)cyclopropane-1-carboxamide (I-90)

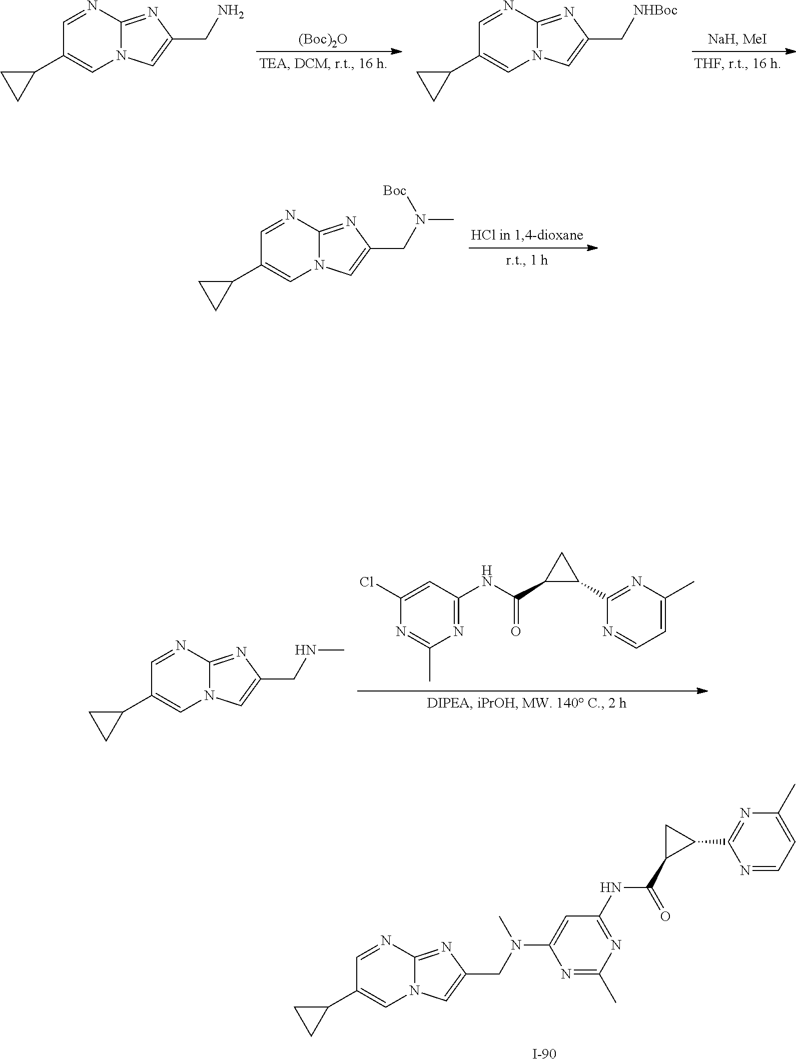

Synthesis of tert-butyl ((6-cyclopropylimidazo[1,2-a]pyrimidin-2-yl)methyl)carbamate. A mixture of (6-cyclopropylimidazo[1,2-a]pyrimidin-2-yl)methanamine (134 mg, 0.71 mmol), (Boc)₂O (310 mg, 1.42 mmol) and TEA (215 mg, 2.13 mmol) in DCM (5 mL) was stirred at room temperature for 16 h. The mixture was concentrated in vacuo to give the crude product, which was purified by column chromatography (eluent: PE/EtOAc=0-10%) to give tert-butyl ((6-cyclopropylimidazo[1,2-a]pyrimidin-2-yl)methyl)carbamate (105 mg, 51%) as a yellow oil. ESI-MS [M+H]⁺: 289.2.

Synthesis of tert-butyl ((6-cyclopropylimidazo[1,2-a]pyrimidin-2-yl)methyl)(methyl) carbamate. To a mixture of tert-butyl ((6-cyclopropylimidazo[1,2-a]pyrimidin-2-yl)methyl)carbamate (50 mg, 0.17 mmol) in THF (2 mL) was added NaH (60% in mineral oil, 14 mg, 0.34 mmol) at 0° C. The mixture was stirred at room temperature for 1 h. Then a solution of Mel (37 mg, 0.26 mmol) in THF (1 mL) was added and the mixture was stirred at room temperature for 16 h. The mixture was quenched with H₂O (30 mL) and extracted with EtOAc (20×3 mL). The combined organic layers were washed with brine (30 mL), dried over anhydrous Na₂SO₄ and concentrated in vacuo to give the crude product, which was purified by preparative TLC (eluent: DCM/MeOH=20/1) to give tert-butyl ((6-cyclopropylimidazo[1,2-a]pyrimidin-2-yl)methyl)(methyl)carbamate (36 mg, 71%) as a yellow oil. ESI-MS [M+H]⁺: 303.2.

Synthesis of 1-(6-cyclopropylimidazo[1,2-a]pyrimidin-2-yl)-N-methylmethanamine. A mixture of tert-butyl ((6-cyclopropylimidazo[1,2-a]pyrimidin-2-yl)methyl)(methyl)carbamate (36 mg, 0.12 mmol) in 1,4-dioxane (2 mL) was added HCl (4 M solution in 1,4-dioxane, 2 mL) at room temperature. The mixture was stirred at room temperature for 1 h. The mixture was quenched with NaHCO₃ (sat.aq., 30 mL) and extracted with DCM (20×3 mL). The combined organic layers were washed with brine (30 mL), dried over anhydrous Na₂SO₄, and concentrated in vacuo to give the crude product, which was purified by preparative TLC (eluent: DCM/MeOH=10/1) to give 1-(6-cyclopropylimidazo[1,2-a]pyrimidin-2-yl)-N-methylmethanamine (20 mg, 83%) as a white solid. ESI-MS [M+H]⁺: 202.2.

Synthesis of (1S,2S)—N-(6-(((6-cyclopropylimidazo[1,2-a]pyrimidin-2-yl)methyl)(methyl)amino)-2-methylpyrimidin-4-yl)-2-(4-methylpyrimidin-2-yl)cyclopropane-1-carboxamide. A mixture of 1-(6-cyclopropylimidazo[1,2-a]pyrimidin-2-yl)-N-methylmethanamine (20 mg, 0.1 mmol), (1S,2S)—N-(6-chloro-2-methylpyrimidin-4-yl)-2-(4-methylpyrimidin-2-yl)cyclopropane-1-carboxamide (45 mg, 0.15 mmol) and DIPEA (65 mg, 0.5 mmol) in i-PrOH (2 mL) was stirred in a sealed tube. After degassing with N₂ for 1 min, the reaction was irradiated in a microwave reactor at 140° C. for 2 h. The reaction was concentrated in vacuo to give the crude product, which was purified by preparative TLC (eluent: DCM/MeOH=20/1) to give (1S,2S)—N-(6-(((6-cyclopropylimidazo[1,2-a]pyrimidin-2-yl)methyl)(methyl)amino)-2-methylpyrimidin-4-yl)-2-(4-methylpyrimidin-2-yl)cyclopropane-1-carboxamide (25.6 mg, 55%) as a white solid. ESI-MS [M+H]⁺: 470.2. ¹H NMR (400 MHz, DMSO) δ 10.77 (s, 1H), 8.60 (d, J=2.4 Hz, 1H), 8.49 (d, J=5.1 Hz, 1H), 8.35 (d, J=2.5 Hz, 1H), 7.50 (s, 1H), 7.22 (s, 1H), 7.17 (d, J=5.1 Hz, 1H), 4.82 (s, 2H), 3.05 (s, 3H), 2.60-2.56 (m, 1H), 2.53-2.50 (m, 1H), 2.38 (s, 3H), 2.28 (s, 3H), 1.98-1.91 (m, 1H), 1.52-1.43 (m, 2H), 0.96-0.91 (m, 2H), 0.73-0.69 (m, 2H).

### Example 91

### Synthesis of (1S,2S)—N-(6-(((3-chloro-8-fluoroquinolin-6-yl)methyl)amino)-2-methylpyrimidin-4-yl-2-(4-methylpyrimidin-2-yl)cyclopropane-1-carboxamide (I-91)

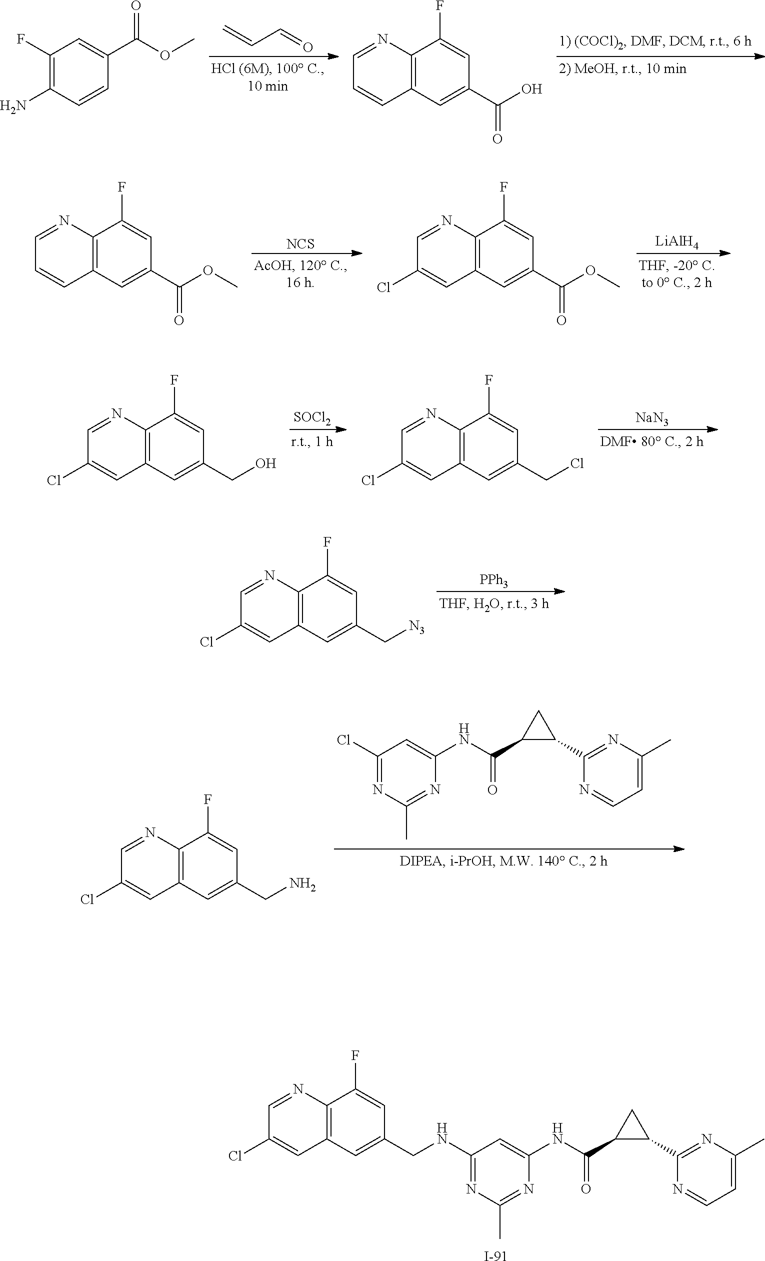

Synthesis of 8-fluoroquinoline-6-carboxylic acid. A mixture of methyl 4-amino-3-fluorobenzoate (10.0 g, 59.2 mmol) and acrylaldehyde (9.9 g, 177.6 mmol) in HCl (6M solution in water, 50 mL) was stirred at 100° C. for 10 min. The mixture was concentrated in vacuo to give 8-fluoroquinoline-6-carboxylic acid (10.0 g, crude) as a yellow solid. ESI-MS [M+H]⁺: 192.2.

Synthesis of methyl 8-fluoroquinoline-6-carboxylate. To a mixture of 8-fluoroquinoline-6-carboxylic acid (10 g, crude) and DMF (0.5 mL) in DCM (100 mL) was added oxalyl chloride (13.2 g, 104.8 mmol) dropwise at 0° C. The mixture was stirred at room temperature for 6 h. Then MeOH (100 mL) was added and the mixture was stirred for 10 min at room temperature. The mixture was concentrated in vacuo to give the crude product, which was purified by column chromatography (eluent: PE/EtOAc=0-20%) to give methyl 8-fluoroquinoline-6-carboxylate (2.51 g, 21% for 2 steps) as a yellow solid. ESI-MS [M+H]⁺: 206.2.

Synthesis of methyl 3-chloro-8-fluoroquinoline-6-carboxylate. To a mixture of methyl 8-fluoroquinoline-6-carboxylate (2.51 g, 12.2 mmol) in AcOH (50 mL) was added NCS (8.11 g, 61.0 mmol) at room temperature. The mixture was stirred at 120° C. for 16 h. The mixture was concentrated, diluted with H₂O (100 mL) and extracted with EtOAc (50 mL×3). The combined organic layers were washed with brine (100 mL), dried over anhydrous Na₂SO₄, and concentrated in vacuo to give the crude product, which was purified by column chromatography (eluent: PE/EtOAc=0˜10%) to give methyl 3-chloro-8-fluoroquinoline-6-carboxylate (2.45 g, 83%) as a yellow solid. ESI-MS [M+H]⁺: 240.1.

Synthesis of (3-chloro-8-fluoroquinolin-6-yl)methanol. To a mixture of methyl 3-chloro-8-fluoroquinoline-6-carboxylate (4.1 g, 17.2 mmol) in THF (100 mL) was added LiAlH₄ (654 mg, 17.2 mmol) dropwise at −20° C. under N₂. The mixture was stirred at 0° C. for 2 h. The reaction was quenched with NH₄Cl (sat. aq., 100 mL) and extracted with EtOAc (50 mL×3). The combined organic layers were washed with brine (100 mL), dried over anhydrous Na₂SO₄, and concentrated in vacuo to give the crude, which was purified by column chromatography (eluent: PE/EtOAc=0˜30%) to give (3-chloro-8-fluoroquinolin-6-yl)methanol (1.0 g, 28%) as a yellow solid. ESI-MS [M+H]⁺: 212.2.

Synthesis of 3-chloro-6-(chloromethyl)-8-fluoroquinoline. To a mixture of (3-chloro-8-fluoroquinolin-6-yl)methanol (200 mg, 0.95 mmol) in DCM (5 mL) was added SOCl₂ (336 mg, 2.85 mmol) dropwise at 0° C. The mixture was stirred at room temperature for 1 h. The mixture was concentrated in vacuo to give 3-chloro-6-(chloromethyl)-8-fluoroquinoline (215 mg, quant.) as a white solid. ESI-MS [M+H]⁺: 230.1.

Synthesis of 6-(azidomethyl)-3-chloro-8-fluoroquinoline. To a mixture of 3-chloro-6-(chloromethyl)-8-fluoroquinoline (160 mg, 0.70 mmol) in DMF (5 mL) was added NaN₃ (68 mg, 1.05 mmol) at room temperature. The mixture was stirred at 80° C. for 2 h. The reaction mixture was cooled to room temperature. The mixture was quenched with H₂O (30 mL) and extracted with EtOAc (20×3 mL). The combined organic layers were washed with brine (30 mL), dried over anhydrous Na₂SO₄ and concentrated in vacuo to give the crude, which was purified by column chromatography (eluent: PE/EtOAc=5/1) to give 6-(azidomethyl)-3-chloro-8-fluoroquinoline (140 mg, 85%) as a yellow solid. ESI-MS [M+H]⁺: 237.2.

Synthesis of (3-chloro-8-fluoroquinolin-6-yl)methanamine. A mixture of 6-(azidomethyl)-3-chloro-8-fluoroquinoline (104 mg, 0.44 mmol) and PPh₃ (231 mg, 0.88 mmol) in THF/H₂O (5 mL/0.1 mL) was stirred at room temperature for 3 h. The mixture was concentrated in vacuo to give the crude product, which was purified by preparative TLC (eluent: DCM/MeOH=5%) to give (3-chloro-8-fluoroquinolin-6-yl)methanamine (49 mg, 53%) as a yellow solid. ESI-MS [M+H]⁺: 211.2.

Synthesis of (1S,2S)—N-(6-(((3-chloro-8-fluoroquinolin-6-yl)methyl)amino)-2-methylpyrimidin-4-yl)-2-(4-methylpyrimidin-2-yl)cyclopropane-1-carboxamide. A mixture of (3-chloro-8-fluoroquinolin-6-yl)methanamine (49 mg, 0.23 mmol), (1S,2S)—N-(6-chloro-2-methylpyrimidin-4-yl)-2-(4-methylpyrimidin-2-yl)cyclopropane-1-carboxamide (70 mg, 0.23 mmol) and DIPEA (89 mg, 0.69 mmol) in i-PrOH (1.5 mL) was stirred in a sealed tube. After degassing with N₂ for 1 min, the reaction was irradiated in a microwave reactor at 140° C. for 2 h. The mixture was concentrated in vacuo to give the crude product, which was purified by preparative TLC (eluent: DCM/MeOH=15/1) to give (1S,2S)—N-(6-(((3-chloro-8-fluoroquinolin-6-yl)methyl)amino)-2-methylpyrimidin-4-yl)-2-(4-methylpyrimidin-2-yl)cyclopropane-1-carboxamide (23.5 mg, 21%) as a white solid. ESI-MS [M+H]⁺: 478.2. ¹H NMR (400 MHz, DMSO) δ 10.70 (s, 1H), 8.89-8.87 (m, 1H), 8.65 (s, 1H), 8.50-8.48 (m, 1H), 7.94-7.89 (m, 1H), 7.66 (s, 1H), 7.59-7.55 (m, 1H), 7.19-7.10 (m, 2H), 4.68 (s, 2H), 2.58-2.57 (m, 1H), 2.54-2.51 (m, 1H), 2.39 (s, 3H), 2.24 (s, 3H), 1.52-1.43 (m, 2H).

### Example 92

### Synthesis of (1S,2S)—N-(5-((3-chloro-8-fluoroquinolin-6-yl)methoxy)pyridazin-3-yl)-2-(4-methylpyrimidin-2-yl)cyclopropane-1-carboxamide (1-92)

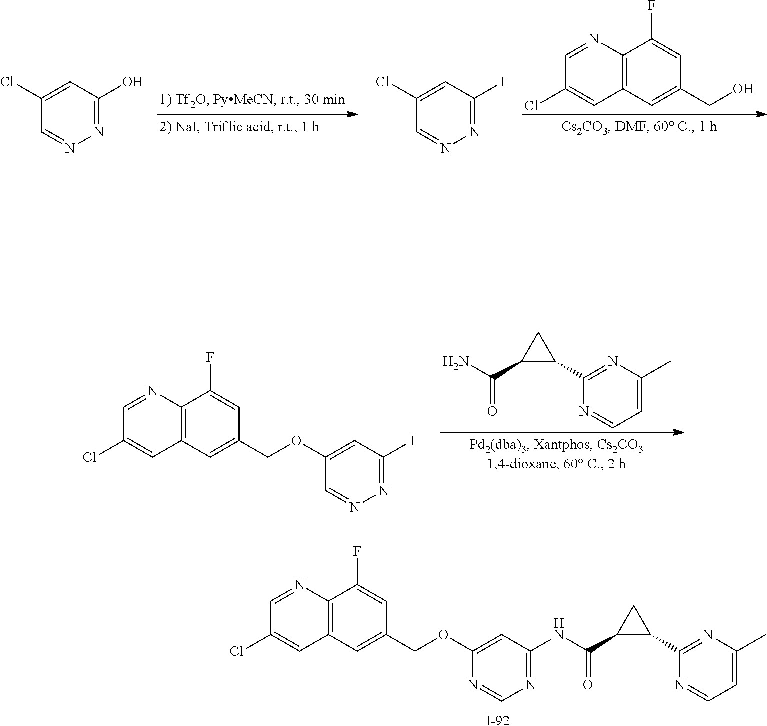

Synthesis of 5-chloro-3-iodopyridazine. To a mixture of 5-chloropyridazin-3-ol (1 g, 7.7 mmol) and pyridine (727 mg, 9.2 mmol) in MeCN (20 mL) was added Tf₂O (2.40 g, 8.5 mmol) dropwise at 0° C. The mixture was stirred at room temperature for 0.5 h. Then a mixture of NaI (5.78 g, 38.5 mmol) and triflic acid (1.28 g, 8.5 mmol) was added at 0° C. dropwise. The mixture was stirred at room temperature for 1 h. The mixture was then quenched with Na₂CO₃ (sat. aq., 100 mL) and extracted with EtOAc (50×3 mL). The combined organic layers were washed with brine (100 mL), dried over anhydrous Na₂SO₄ and concentrated in vacuo to give the crude, which was purified by column chromatography (eluent: PE/EtOAc=0˜30%) to give 5-chloro-3-iodopyridazine (904 mg, 49%) as a yellow solid. ESI-MS [M+H]⁺: 240.9.

Synthesis of 3-chloro-8-fluoro-6-(((6-iodopyridazin-4-yl)oxy)methyl)quinolone. A mixture of (3-chloro-8-fluoroquinolin-6-yl)methanol (250 mg, 1.2 mmol), 5-chloro-3-iodopyridazine (288 mg, 1.2 mmol) and Cs₂CO₃ (1.17 g, 3.6 mmol) in DMF (10 mL) was stirred at 60° C. for 1 h. After cooling to room temperature, H₂O (50 mL) was added and the mixture was extracted with EtOAc (30 mL×3). The combined organic layers were washed with brine (60 mL), dried over anhydrous Na₂SO₄ and concentrated in vacuo to give the crude, which was purified by column chromatography (eluent: PE/EtOAc=0˜30%) to give 3-chloro-8-fluoro-6-(((6-iodopyridazin-4-yl)oxy)methyl)quinoline (168 mg, 34%) as a yellow solid. ESI-MS [M+H]⁺: 415.9.

Synthesis of (1S,2S)—N-(5-((3-chloro-8-fluoroquinolin-6-yl)methoxy)pyridazin-3-yl)-2-(4-methylpyrimidin-2-yl)cyclopropane-1-carboxamide. A mixture of 3-chloro-8-fluoro-6-(((6-iodopyridazin-4-yl)oxy)methyl)quinoline (100 mg, 0.24 mmol), (1S,2S)-2-(4-methylpyrimidin-2-yl)cyclopropane-1-carboxamide (43 mg, 0.24 mmol), and Cs₂CO₃ (235 mg, 0.72 mmol) in 1,4-dioxane (2 mL) was added Pd₂(dba)₃ (22 mg, 0.024 mmol) and Xantphos (28 mg, 0.048 mmol). The mixture was stirred at 60° C. for 2 h under N₂. The reaction mixture was cooled to room temperature. The mixture was filtered through Celite® and the filter cake was washed with DCM/MeOH (10/1, 30 mL). The filtrate was concentrated in vacuo to give the crude, which was purified by preparative TLC (eluent: DCM/MeOH=20/1) to give (1S,2S)—N-(5-((3-chloro-8-fluoroquinolin-6-yl)methoxy)pyridazin-3-yl)-2-(4-methylpyrimidin-2-yl)cyclopropane-1-carboxamide (21.2 mg, 19%) as a white solid. ESI-MS [M+H]⁺: 465.2. ¹H NMR (400 MHz, DMSO) δ 11.46 (s, 1H), 8.98 (d, J=2.3 Hz, 1H), 8.86 (d, J=2.7 Hz, 1H), 8.72 (s, 1H), 8.54 (d, J=5.1 Hz, 1H), 8.06 (d, J=2.7 Hz, 1H), 7.94 (s, 1H), 7.78 (dd, J=11.4, 1.4 Hz, 1H), 7.23 (d, J=5.1 Hz, 1H), 5.48 (s, 2H), 2.72-2.69 (m, 1H), 2.60-2.58 (m, 1H), 2.43 (s, 3H), 1.61-1.54 (m, 2H).

### Example 93

### Synthesis of (1S,2S)—N-(6-(((3-cyclopropylquinolin-6-yl)methyl)amino)pyrimidin-4-yl)-2-(4-methylpyrimidin-2-yl)cyclopropane-1-carboxamide (I-93)

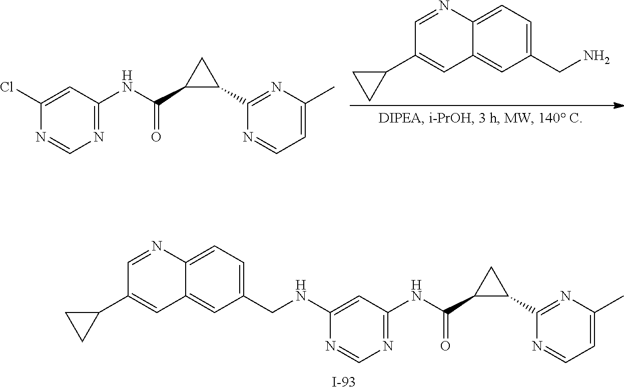

A mixture of (3-cyclopropylquinolin-6-yl)methanamine (70 mg, 0.35 mmol), (1S,2S)—N-(6-chloropyrimidin-4-yl)-2-(4-methylpyrimidin-2-yl)cyclopropane-1-carboxamide (58 mg, 0.2 mmol) and DIPEA (142 mg, 1.1 mmol) in i-PrOH (3 mL) was stirred in a sealed tube. The reaction was irradiated in a microwave reactor at 140° C. for 3 h. After cooling to room temperature, the reaction was concentrated in vacuo to give the crude product, which was purified by preparative TLC (eluent: DCM/MeOH=10/1) to give (1S,2S)—N-(6-(((3-cyclopropylquinolin-6-yl)methyl)amino)pyrimidin-4-yl)-2-(4-methylpyrimidin-2-yl)cyclopropane-1-carboxamide (50 mg, 56%) as a white solid. ESI-MS [M+H]⁺: 452.2. ¹H NMR (400 MHz, DMSO) δ 10.70 (s, 1H), 8.71 (d, J=2.2 Hz, 1H), 8.52 (d, J=5.1 Hz, 1H), 8.18 (s, 1H), 8.09-8.00 (m, 1H), 7.92-7.89 (m, 2H), 7.70 (s, 1H), 7.59 (dd, J=8.6, 1.7 Hz, 1H), 7.32 (s, 1H), 7.21 (d, J=5.1 Hz, 1H), 4.69 (s, 2H), 2.64-2.60 (m, 1H), 2.57-2.53 (m, 1H), 2.41 (s, 3H), 2.15-2.08 (m, 1H), 1.56-1.49 (m, 2H), 1.09-1.04 (m, 2H), 0.89-0.85 (m, 2H).

### Example 94

### Synthesis of (1S,2S)—N-(5-((6-cyclopropylimidazo[1,2-a]pyrimidin-2-yl)methoxy)pyridazin-3-yl)-2-(4-methylpyrimidin-2-yl)cyclopropane-1-carboxamide (I-94)

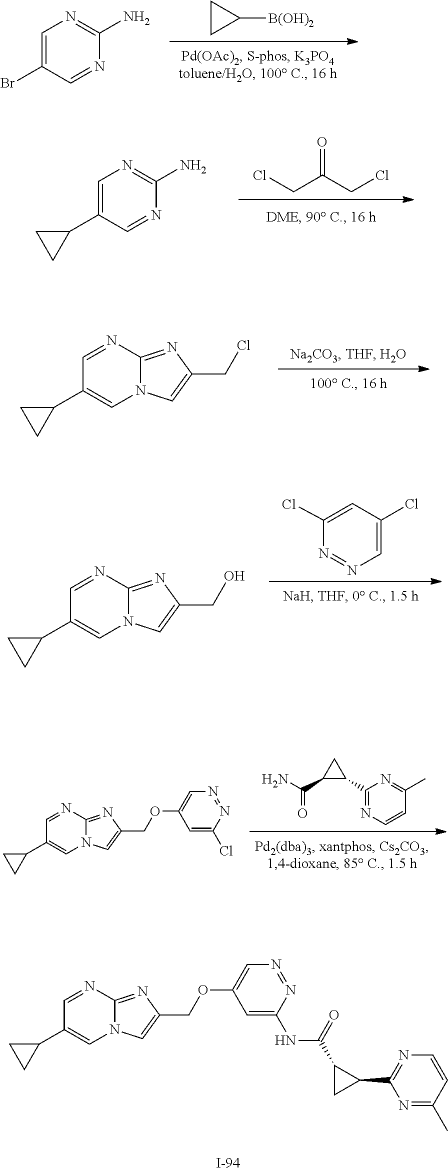

Synthesis of 5-cyclopropylpyrimidin-2-amine. To a mixture of 5-bromopyrimidin-2-amine (5 g, 28.7 mmol) in toluene/H₂O (100 mL/10 mL) was added cyclopropylboronic acid (7.4 g, 86.2 mmol), Pd(OAc)₂ (642.9 mg, 2.87 mmol), S-phos (1.18 g, 2.87 mmol), K₃PO₄ (18.3 g, 86.2 mmol) at room temperature. The reaction mixture was stirred at 100° C. for 16 h under N₂. The reaction mixture cooled to room temperature, filtered and the filter cake was washed with DCM/MeOH (10/1, 200 mL). The filtrate was concentrated in vacuo to give the crude, which was purified by silica gel chromatography eluting with a gradient of 0-10% EtOAc in PE to give 5-cyclopropylpyrimidin-2-amine (2.8 g, 72%) as a yellow solid. ESI-MS [M+H]⁺: 136.0

Synthesis of 2-(chloromethyl)-6-cyclopropylimidazo[1,2-a]pyrimidine. To a mixture of 5-cyclopropylpyrimidin-2-amine (2 g, 14.8 mmol) in DME (50 mL) was added 1,3-dichloropropan-2-one (5.63 g, 44.4 mmol). The resulting mixture was stirred at 90° C. for 0.5 h. The reaction mixture was cooled to room temperature, poured into water (50 mL) and extracted with DCM (60 mL×3). The combined organics were washed with brine (100 mL), dried over Na₂SO₄ and concentrated in vacuo. The residue was purified by silica gel chromatography eluting with a gradient of 0-10% MeOH in DCM to give 2-(chloromethyl)-6-cyclopropylimidazo[1,2-a]pyrimidine (1.4 g, 46%) as a yellow solid. ESI-MS [M+H]⁺: 208.0

Synthesis of (6-cyclopropylimidazo[1,2-a]pyrimidin-2-yl)methanol. To a mixture of 2-(chloromethyl)-6-cyclopropylimidazo[1,2-a]pyrimidine (700 mg, 3.37 mmol) in THF/H₂O (75 mL/75 mL) was added Na₂CO₃ (1.78 g, 16.85 mmol). The resulting mixture was stirred at 100° C. for 16 h. The reaction mixture was cooled to room temperature, extracted with EtOAc (100 mL×3). The combined organics were washed with brine (100 mL), dried over Na₂SO₄ and concentrated in vacuo. The residue was purified by silica gel chromatography eluting with a gradient of 0-10% MeOH in DCM to give (6-cyclopropylimidazo[1,2-a]pyrimidin-2-yl)methanol (155 mg, 24%) as yellow oil. ESI-MS [M+H]⁺: 190.1

Synthesis of 2-(((6-chloropyridazin-4-yl)oxy)methyl)-6-cyclopropylimidazo[1,2-a]pyrimidine. To a mixture of (6-cyclopropylimidazo[1,2-a]pyrimidin-2-yl)methanol (100 mg, 0.53 mmol) in THF (5 mL) was added NaH (42 mg, 1.05 mmol, 60% dispersion in mineral oil) at 0° C. and the mixture was stirred at 0° C. for 0.5 h. Then 3,5-dichloropyridazine (157.5 mg, 1.06 mmol) was added thereto and the reaction was stirred at room temperature for 1 h. The reaction mixture was diluted with H₂O (20 mL) and extracted with EtOAc (30 mL×3). The combined organics were washed with brine (100 mL), dried over Na₂SO₄ and concentrated in vacuo. The residue was purified by preparative TLC (eluent: DCM:MeOH=20:1) to give 2-(((6-chloropyridazin-4-yl)oxy)methyl)-6-cyclopropylimidazo[1,2-a]pyrimidine (73 mg, yield 45%) as a yellow solid. ESI-MS [M+H]⁺: 302.0

Synthesis of (1S,2S)—N-(5-((6-cyclopropylimidazo[1,2-a]pyrimidin-2-yl)methoxy)pyridazin-3-yl)-2-(4-methylpyrimidin-2-yl)cyclopropane-1-carboxamide. To a solution of 2-(((6-chloropyridazin-4-yl)oxy)methyl)-6-cyclopropylimidazo[1,2-a]pyrimidine (35 mg, 0.12 mmol) in 1,4-dioxane (7 mL) was added (1S,2S)-2-(4-methylpyrimidin-2-yl)cyclopropane-1-carboxamide (20.6 mg, 0.12 mmol), Pd₂(dba)₃ (21.2 mg, 0.023 mmol), xantphos (26.6 mg, 0.046 mmol), Cs₂CO₃ (114 mg, 0.35 mmol). The reaction mixture was stirred at 85° C. for 1.5 h under N₂. The reaction mixture was cooled to room temperature and filtered. The filter cake was washed with DCM/MeOH (10/1, 20 mL). The filtrate was concentrated in vacuo to give the crude, which was purified by preparative TLC (eluent: DCM/MeOH=15/1) to give (1S,2S)—N-(5-((6-cyclopropylimidazo[1,2-a]pyrimidin-2-yl)methoxy)pyridazin-3-yl)-2-(4-methylpyrimidin-2-yl)cyclopropane-1-carboxamide (18.3 mg, 36%) as a white solid. ESI-MS [M+H]⁺: 443.2, ¹H NMR (400 MHz, DMSO) δ 11.42 (s, 1H), 8.82 (d, J=2.8 Hz, 1H), 8.74 (d, J=2.5 Hz, 1H), 8.54 (d, J=5.1 Hz, 1H), 8.45 (d, J=2.5 Hz, 1H), 8.05 (d, J=2.7 Hz, 1H), 7.88 (s, 1H), 7.22 (d, J=5.1 Hz, 1H), 5.38 (s, 2H), 2.71-2.67 (m, 1H), 2.62-2.57 (m, 1H), 2.43 (s, 3H), 2.04-1.97 (m, 1H), 1.61-1.54 (m, 2H), 1.01-0.96 (m, 2H), 0.80-0.76 (m, 2H).

### Example 95

### Synthesis of (S,2S)—N-(6-(((R^{•})-1-(5-cyclopropyl-7-(3-methyl-2,4-dioxoimidazolidin-1-yl)pyrazolo[1,5-a]pyridin-2-yl)ethyl)amino)pyrimidin-4-yl)-2-(4-methylpyrimidin-2-yl)cyclopropane-1-carboxamide, isomer 1 (I-95)

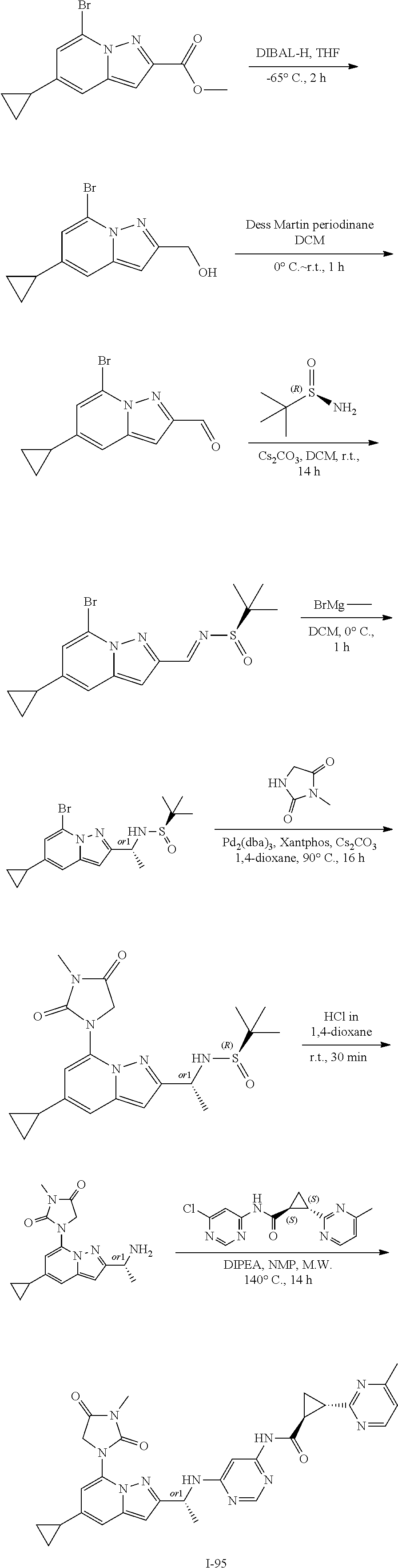

Synthesis of (7-bromo-5-cyclopropylpyrazolo[1,5-a]pyridin-2-yl)methanol. To a stirred solution of methyl 7-bromo-5-cyclopropylpyrazolo[1,5-a]pyridine-2-carboxylate (1.0 g, 3.4 mmol) in THF (20 mL) was added dropwise DIBAL-H (10 mL, 1M in hexanes, 10 mmol) at −65° C. The mixture was stirred at −65° C. for 2 h. The reaction mixture was quenched with NaOH (1 M aq., 20 mL) at −65° C., and then warmed to room temperature. After stirring 1 h, the mixture was extracted with EtOAc (50 mL×2). The combined organics were washed with brine (100 mL), dried over Na₂SO₄, and then concentrated in vacuo. The residue was purified by column chromatography on silica gel, eluting with 0˜20% EtOAc in PE to give (7-bromo-5-cyclopropylpyrazolo[1,5-a]pyridin-2-yl)methanol (0.80 g, yield 88%) as a yellow solid. ESI-MS [M+H]⁺: 267.0.

Synthesis of 7-bromo-5-cyclopropylpyrazolo[1,5-a]pyridine-2-carbaldehyde. To a stirred solution of (7-bromo-5-cyclopropylpyrazolo[1,5-a]pyridin-2-yl)methanol (0.80 g, 3.0 mmol) in DCM (20 mL) was added Dess Martin periodinane (2.5 g 6.0 mmol) at 0° C. The mixture was stirred at room temperature for 1 h. The reaction mixture was diluted with DCM (30 mL) and quenched with Na₂S₂O₃ (50 mL, 10% aq.). The organic layer was washed with NaHCO₃ (sat. aq., 50 mL) and brine (50 mL), dried over anhydrous Na₂SO₄, and then concentrated in vacuo. The residue was purified by column chromatography on silica gel, eluting with 0-10% EtOAc in PE to give 7-bromo-5-cyclopropylpyrazolo[1,5-a]pyridine-2-carbaldehyde (0.71 g, 90%) as a yellow solid. ESI-MS [M+H]⁺: 265.0.

Synthesis of (R,E)-N-((7-bromo-5-cyclopropylpyrazolo[1,5-a]pyridin-2-yl)methylene)-2-methylpropane-2-sulfinamide. To a stirred solution of 7-bromo-5-cyclopropylpyrazolo[1,5-a]pyridine-2-carbaldehyde (0.71 g, 2.7 mmol) and (R)-2-methylpropane-2-sulfinamide (0.39 g, 3.2 mmol) in DCM (20 mL) was added Cs₂CO₃ (1.8 g, 5.4 mmol) at room temperature. The mixture was stirred at room temperature for 14 h. The reaction mixture was filtered and washed with DCM (30 mL). The filtrate was concentrated in vacuo. The residue was purified by column chromatography on silica gel, eluting with 0-20% EtOAc in PE to give (R,E)-N-((7-bromo-5-cyclopropylpyrazolo[1,5-a]pyridin-2-yl)methylene)-2-methylpropane-2-sulfinamide (0.99 g, quant) as a yellow solid. ESI-MS [M+H]⁺: 368.0.

Synthesis of (R)—N—((R^{•})-1-(7-bromo-5-cyclopropylpyrazolo[1,5-a]pyridin-2-yl)ethyl)-2-methylpropane-2-sulfinamide. To a stirred solution of (R,E)-N-((7-bromo-5-cyclopropylpyrazolo[1,5-a]pyridin-2-yl)methylene)-2-methylpropane-2-sulfinamide (0.88 g, 2.4 mmol) in DCM (30 mL) was added dropwise CH₃MgBr (1.6 mL, 3 M in diethyl ether, 4.8 mmol) at 0° C. The mixture was stirred at 0° C. for 1 h. The reaction mixture was quenched with NH₄Cl (sat. aq., 20 mL) and extracted with DCM (30 mL×2). The combined organics were washed with brine (80 mL), dried over anhydrous Na₂SO₄, and then concentrated in vacuo. The residue was purified by column chromatography on silica gel, eluting with 30˜50% EtOAc in PE to give (R)—N—((R *)-1-(7-bromo-5-cyclopropylpyrazolo[1,5-a]pyridin-2-yl)ethyl)-2-methylpropane-2-sulfinamide, isomer 1 (0.53 g, yield 58%) and (R)—N—((R^{•})-1-(7-bromo-5-cyclopropylpyrazolo[1,5-a]pyridin-2-yl)ethyl)-2-methylpropane-2-sulfinamide, isomer 2 (0.21 g, 23%) as yellow solids. ESI-MS [M+H]⁺: 384.1.

Synthesis of (R)—N—((R^{•})-1-(5-cyclopropyl-7-(3-methyl-2,4-dioxoimidazolidin-1-yl)pyrazolo[1,5-a]pyridin-2-yl)ethyl)-2-methylpropane-2-sulfinamide, isomer 1. A solution of (R)—N—((R*)-1-(7-bromo-5-cyclopropylpyrazolo[1,5-a]pyridin-2-yl)ethyl)-2-methylpropane-2-sulfinamide, isomer 1 (0.53 g, 1.4 mmol), 3-methylimidazolidine-2,4-dione (0.47 g, 4.1 mmol), Pd₂(dba)₃ (0.13 g, 0.14 mmol), Xantphos (0.16 g, 0.28 mmol) and Cs₂CO₃ (1.34 g, 4.1 mmol) in 1,4-dioxane (20 mL) was stirred at 90° C. for 16 h under N₂ atmosphere. The mixture was cooled to room temperature. The reaction mixture was filtered through Celite®, and the filter cake was washed with DCM/MeOH (v/v=10/1, 50 mL). The filtrate was concentrated in vacuo. The residue was purified by column chromatography on silica gel, eluting with 20˜50% EtOAc in PE to give (R)—N—((R^{•})-1-(5-cyclopropyl-7-(3-methyl-2,4-dioxoimidazolidin-1-yl)pyrazolo[1,5-a]pyridin-2-yl)ethyl)-2-methylpropane-2-sulfinamide, isomer 1 (0.45 g, yield 77%) as a yellow solid. ESI-MS [M+H]⁺: 418.2.

Synthesis of (R^{•})-1-(2-(1-aminoethyl)-5-cyclopropylpyrazolo[1,5-a]pyridin-7-yl)-3-methylimidazolidine-2,4-dione, isomer 1. To a solution of (R)—N—((R^{•})-1-(5-cyclopropyl-7-(3-methyl-2,4-dioxoimidazolidin-1-yl)pyrazolo[1,5-a]pyridin-2-yl)ethyl)-2-methylpropane-2-sulfinamide, isomer 1 (0.15 g, 0.36 mmol) in MeOH (3 mL) was added HCl (4 M in 1,4-dioxane, 2.0 mL). The resulting mixture was stirred at room temperature for 30 min. The reaction mixture was concentrated in vacuo and diluted with NH₃ (7 N in MeOH, 5.0 mL). The mixture was stirred for another 10 min and then concentrated in vacuo. The residue was purified by preparative TLC, eluting with 5% MeOH in DCM to give (R *)-1-(2-(1-aminoethyl)-5-cyclopropylpyrazolo[1,5-a]pyridin-7-yl)-3-methylimidazolidine-2,4-dione, isomer 1 (90 mg, yield 80%) as a yellow solid. ESI-MS [M+H]⁺: 314.2.

Synthesis of (S,2S)—N-(6-(((R^{•})-1-(5-cyclopropyl-7-(3-methyl-2,4-dioxoimidazolidin-1-yl)pyrazolo[1,5-a]pyridin-2-yl)ethyl)amino)pyrimidin-4-yl)-2-(4-methylpyrimidin-2-yl)cyclo propane-1-carboxamide, isomer 1. A solution of (R^{•})-1-(2-(1-aminoethyl)-5-cyclopropylpyrazolo[1,5-a]pyridin-7-yl)-3-methylimidazolidine-2,4-dione, isomer 1 (90 mg, 0.29 mmol), (1S,2S)—N-(6-chloropyrimidin-4-yl)-2-(4-methylpyrimidin-2-yl)cyclopropane-1-carboxamide (83 mg, 0.29 mmol) and DIPEA (0.37 g, 2.9 mmol) in NMP (4.0 mL) was stirred in a sealed tube. After degassing with N₂ for 1 min, the reaction was irradiated in a microwave reactor at 140° C. for 14 h. The mixture was cooled to room temperature. The reaction mixture was poured into water (20 mL), and then extracted with EtOAc (20 mL×3). The combined organics were washed with water (50 mL) and brine (50 mL), dried over Na₂SO₄, and concentrated in vacuo. The residue was purified by column chromatography on silica gel, eluting with 0˜5% MeOH in DCM to give (1S,2S)—N-(6-(((R *)-1-(5-cyclopropyl-7-(3-methyl-2,4-dioxoimidazolidin-1-yl)pyrazolo[1,5-a]pyridin-2-yl)ethyl)amino)pyrimidin-4-yl)-2-(4-methylpyrimidin-2-yl)cyclopropane-1-carboxamide, isomer 1 (40 mg, yield 24%) as a white solid. ESI-MS [M+H]⁺: 567.2. ¹H NMR (400 MHz, DMSO) δ 10.64 (s, 1H), 8.52 (d, J=5.1 Hz, 1H), 8.15 (s, 1H), 7.81 (d, J=8.3 Hz, 1H), 7.34 (d, J=1.7 Hz, 1H), 7.29 (s, 1H), 7.20 (d, J=5.1 Hz, 1H), 6.74 (d, J=1.8 Hz, 1H), 6.41 (s, 1H), 5.44 (s, 1H), 4.66 (s, 2H), 3.00 (s, 3H), 2.63-2.53 (m, 2H), 2.41 (s, 3H), 2.03-1.91 (m, 1H), 1.51-1.50 (m, 5H), 1.05-0.97 (m, 2H), 0.78-0.70 (m, 2H).

### Example 96

### Synthesis of (S,2S)—N-(6-(((R^{•})-1-(5-cyclopropyl-7-(3-methyl-2,4-dioxoimidazolidin-1-yl)pyrazolo[1,5-a]pyridin-2-yl)ethyl)amino)pyrimidin-4-yl)-2-(4-methylpyrimidin-2-yl) cyclopropane-1-carboxamide, isomer 2 (I-96)

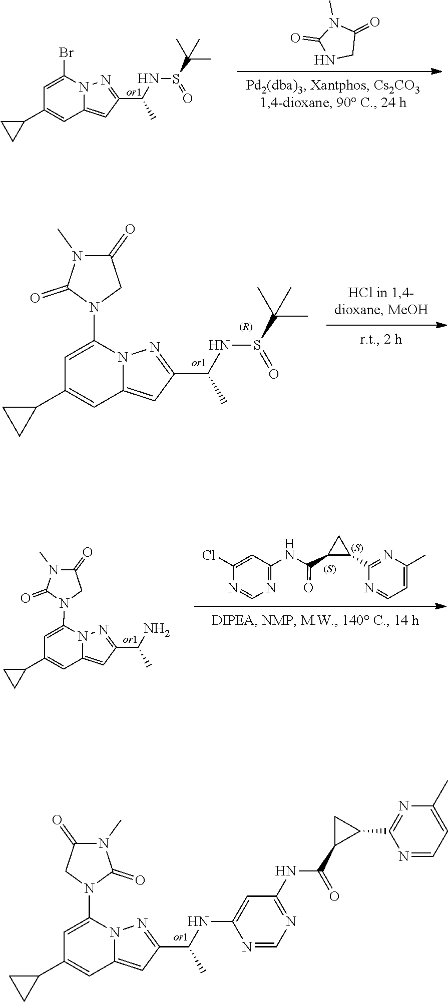

Synthesis of (R)—N—((R^{•})-1-(5-cyclopropyl-7-(3-methyl-2,4-dioxoimidazolidin-1-yl)pyrazolo[1,5-a]pyridin-2-yl)ethyl)-2-methylpropane-2-sulfinamide, isomer 2. A solution of (R)—N—((R^{•})-1-(7-bromo-5-cyclopropylpyrazolo[1,5-a]pyridin-2-yl)ethyl)-2-methylpropane-2-sulfinamide, isomer 2 (0.21 g, 0.55 mmol), 3-methylimidazolidine-2,4-dione (182 mg, 1.6 mmol), Pd₂(dba)₃ (49 mg, 0.053 mmol), Xantphos (64 mg, 0.11 mmol) and Cs₂CO₃ (521 mg, 1.6 mmol) in 1,4-dioxane (8.0 mL) was stirred at 90° C. for 16 h under N₂ atmosphere. The mixture was cooled to room temperature and then filtered through a pad of Celite®. The filtrate was concentrated in vacuo. The residue was purified by column chromatography on silica gel, eluting with 20˜50% EtOAc in PE to give (R)—N—((R^{•})-1-(5-cyclopropyl-7-(3-methyl-2,4-dioxoimidazolidin-1-yl)pyrazolo[1,5-a]pyridin-2-yl)ethyl)-2-methylpropane-2-sulfinamide, isomer 2 (0.17 g, 74%) as a yellow solid. ESI-MS [M+H]⁺: 418.2.

Synthesis of (R^{•})-1-(2-(1-aminoethyl)-5-cyclopropylpyrazolo[1,5-a]pyridin-7-yl)-3-methylimidazolidine-2,4-dione. To a solution of (R)—N—((R^{•})-1-(5-cyclopropyl-7-(3-methyl-2,4-dioxoimidazolidin-1-yl)pyrazolo[1,5-a]pyridin-2-yl)ethyl)-2-methylpropane-2-sulfinamide, isomer 2 (0.17 g, 0.38 mmol) in MeOH (2.0 mL) was added HCl (4 M solution in 1,4-dioxane, 4.0 mL). The resulting mixture was stirred at room temperature for 2 h. The reaction mixture was concentrated in vacuo, and then NH₃ solution (7 M in MeOH, 5.0 mL) was added. The mixture was stirred for another 10 min and then concentrated in vacuo. The residue was purified by preparative TLC, eluting with 10% MeOH in DCM to give (R^{•})-1-(2-(1-aminoethyl)-5-cyclopropylpyrazolo[1,5-a]pyridin-7-yl)-3-methylimidazolidine-2,4-dione, isomer 2 (70 mg, 59%) as a yellow solid. ESI-MS [M+H]⁺: 314.2.

Synthesis of (S,2S)—N-(6-(((R^{•})-1-(5-cyclopropyl-7-(3-methyl-2,4-dioxoimidazolidin-1-yl)pyrazolo[1,5-a]pyridin-2-yl)ethyl)amino)pyrimidin-4-yl)-2-(4-methylpyrimidin-2-yl)cyclopropane-1-carboxamide, isomer 2. A solution of (R^{•})-1-(2-(1-aminoethyl)-5-cyclopropylpyrazolo[1,5-a]pyridin-7-yl)-3-methylimidazolidine-2,4-dione, isomer 2 (70 mg, 0.22 mmol), (1S,2S)—N-(6-chloropyrimidin-4-yl)-2-(4-methylpyrimidin-2-yl)cyclopropane-1-carboxamide (65 mg, 0.22 mmol) and DIPEA (288 mg, 2.23 mmol) in NMP (4.0 mL) was stirred in a sealed tube. After degassing with N₂ for 1 min, the reaction was irradiated in a microwave reactor at 140° C. for 14 h. The reaction mixture was poured into water (20 mL) and extracted with EtOAc (20 ml×3). The combined organics were washed with water (50 mL×3) and brine (50 mL), dried over Na₂SO₄, concentrated in vacuo. The residue was purified by column chromatography on silica gel, eluting with 0-5% MeOH in DCM to give (1S,2S)—N-(6-(((R^{•})-1-(5-cyclopropyl-7-(3-methyl-2,4-dioxoimidazolidin-1-yl)pyrazolo[1,5-a]pyridin-2-yl)ethyl)amino)pyrimidin-4-yl)-2-(4-methylpyrimidin-2-yl)cyclopropane-1-carboxamide, isomer 2 (75 mg, yield 59%) as a white solid. ESI-MS [M+H]⁺: 567.2, ¹H NMR (400 MHz, DMSO) δ 10.65 (s, 1H), 8.52 (d, J=5.1 Hz, 1H), 8.15 (s, 1H), 7.82 (d, J=6.8 Hz, 1H), 7.34 (d, J=1.7 Hz, 1H), 7.29 (s, 1H), 7.20 (d, J=5.1 Hz, 1H), 6.74 (d, J=1.7 Hz, 1H), 6.42 (s, 1H), 5.45 (s, 1H), 4.68 (s, 2H), 3.01 (s, 3H), 2.64-2.54 (m, 2H), 2.41 (s, 3H), 2.03-1.97 (m, 1H), 1.58-1.46 (m, 5H), 1.04-0.99 (m, 2H), 0.76-0.72 (m, 2H).

### Example 97

### Synthesis of (1S,2S)—N-(5-((6-cyclopropyl-8-(3-methyl-2,4-dioxoimidazolidin-1-yl)imidazo[1,2-b]pyridazin-2-yl)methoxy)pyridazin-3-yl)-2-(4-methylpyrimidin-2-yl)cyclopropane-1-carboxamidee (I-97)

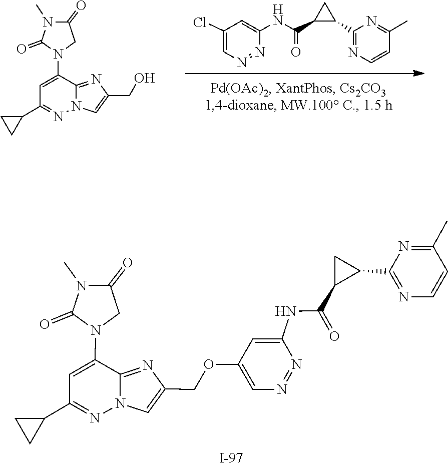

To a mixture of 1-(6-cyclopropyl-2-(hydroxymethyl)imidazo[1,2-b]pyridazin-8-yl)-3-methylimidazolidine-2,4-dione (140 mg, 0.47 mmol), (1S,2S)—N-(5-chloropyridazin-3-yl)-2-(4-methylpyrimidin-2-yl)cyclopropane-1-carboxamide (136 mg, 0.47 mmol) and Cs₂CO₃ (456 mg, 1.4 mmol) in 1,4-dioxane (10 mL) was added Pd(OAc)₂ (21 mg, 0.094 mmol) and Xantphos (110 mg, 0.19 mmol). The reaction was stirred in a sealed tube. After degassing with N₂ for 1 min, the reaction was irradiated in a microwave reactor at 100° C. for 1.5 h. The reaction mixture was cooled to room temperature. The reaction mixture was filtered through Celite® and the filter cake was washed with DCM/MeOH (10/1, 30 mL). The filtrate was concentrated in vacuo to give the crude, which was purified by preparative TLC (eluent: DCM/MeOH=20/1) to give (1S,2S)—N-(5-((6-cyclopropyl-8-(3-methyl-2,4-dioxoimidazolidin-1-yl)imidazo[1,2-b]pyridazin-2-yl)methoxy)pyridazin-3-yl)-2-(4-methylpyrimidin-2-yl)cyclopropane-1-carboxamide (9.7 mg, 4%) as a white solid. ESI-MS [M+H]⁺: 555.2, ¹H NMR (400 MHz, DMSO) δ 11.40 (s, 1H), 8.78 (d, J=2.7 Hz, 1H), 8.54 (d, J=5.1 Hz, 1H), 8.30 (s, 1H), 8.22 (d, J=2.7 Hz, 1H), 7.74 (s, 1H), 7.22 (d, J=5.1 Hz, 1H), 5.38 (s, 2H), 5.18-5.07 (m, 2H), 2.99 (s, 3H), 2.71-2.67 (m, 1H), 2.60-2.56 (m, 1H), 2.42 (s, 3H), 2.20-2.13 (m, 1H), 1.58-1.55 (m, 2H), 1.10-1.05 (m, 2H), 0.96-0.91 (m, 2H).

### Example 98

### Synthesis of (1S,2S)—N-(6-(((3-cyclopropyl-8-(3-methyl-2,4-dioxoimidazolidin-1-yl)quinolin-6-yl)methyl)amino)pyrimidin-4-yl)-2-(4-methylpyrimidin-2-yl)cyclopropane-1-carboxamide (I-98)

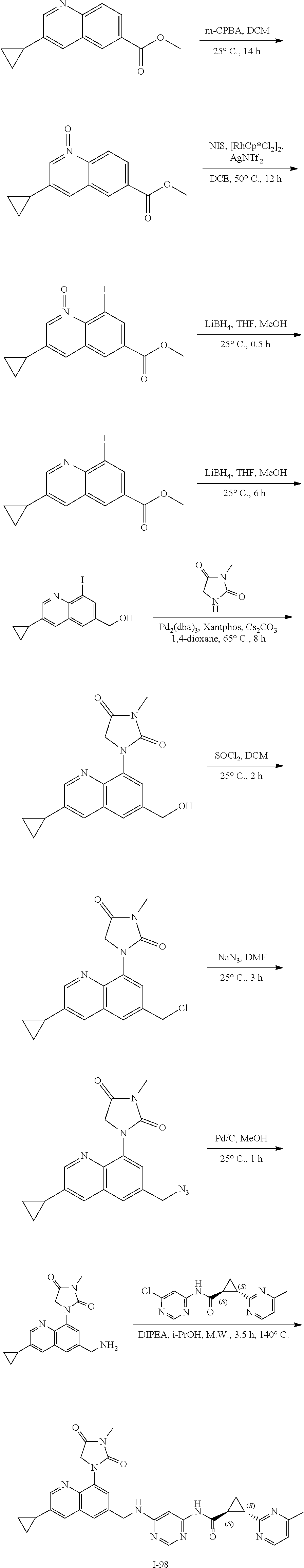

Synthesis of 3-cyclopropyl-6-(methoxycarbonyl)quinoline 1-oxide. A solution of methyl 3-cyclopropylquinoline-6-carboxylate (1.45 g, 6.38 mmol) and m-CPBA (1.32 g, 7.65 mmol) in DCM (40 mL) was stirred at 25° C. for 14 h. The reaction mixture was quenched with Na₂SO₃ (sat, aq., 50 mL) and extracted with DCM (80 mL×3). The combined organic layers were washed with brine (30 mL), dried over anhydrous Na₂SO₄, and concentrated in vacuo. The residue was purified by column chromatography on silica gel, eluting with 10-60% EtOAc in PE to give 3-cyclopropyl-6-(methoxycarbonyl)quinoline 1-oxide (1.3 g, 84%) as a yellow solid. ESI-MS [M+H]⁺: 244.1

Synthesis of 3-cyclopropyl-8-iodo-6-(methoxycarbonyl)quinoline 1-oxide. A solution of 3-cyclopropyl-6-(methoxycarbonyl)quinoline 1-oxide (1 g, 4.1 mmol), NIS (2.97 g, 13.2 mmol), AgNTf₂ (341 mg, 0.88 mmol) and [RhCp*Cl₂]2 (272 mg, 0.44 mmol) in DCE (14 mL) was stirred at 50° C. for 12 h under N₂ atmosphere. The reaction mixture was filtered through a pad of Celite®, and the filter cake was washed with DCM/MeOH (v/v=10/1, 60 mL). The filtrate was concentrated in vacuo. The residue was purified by column chromatography on silica gel, eluting with 0-5% MeOH in DCM to give 3-cyclopropyl-8-iodo-6-(methoxycarbonyl)quinoline 1-oxide (1.0 g, 66%) as a yellow solid. ESI-MS [M+H]⁺: 369.9.

Synthesis of methyl 3-cyclopropyl-8-iodoquinoline-6-carboxylate. To a solution of 3-cyclopropyl-8-iodo-6-(methoxycarbonyl)quinoline 1-oxide (1 g, 2.7 mmol) in THF/MeOH (V/V=2/1, 30 mL) was added LiBH₄ (118 mg, 5.4 mmol). The mixture was stirred at 25° C. for 0.5 h. The reaction mixture was then quenched with water (20 mL) then extracted with EtOAc (50 mL×3). The combined organic layers were washed with brine (30 mL), dried over anhydrous Na₂SO₄, and concentrated in vacuo. The residue was purified by column chromatography on silica gel, eluting with 10-60% EtOAc in PE to give methyl 3-cyclopropyl-8-iodoquinoline-6-carboxylate (600 mg, 63%) as a yellow solid. ESI-MS [M+H]⁺: 353.9.

Synthesis of (3-cyclopropyl-8-iodoquinolin-6-yl)methanol. To a solution of methyl 3-cyclopropyl-8-iodoquinoline-6-carboxylate (600 mg, 1.69 mmol) in THF/MeOH (V/V=2/1, 15 mL) was added LiBH₄ (187 mg, 8.5 mmol). The mixture was stirred at 25° C. for 6 h. The reaction mixture was quenched with water (20 mL) then extracted with EtOAc (50 mL×3). The combined organic layers were washed with brine (30 mL), dried over anhydrous Na₂SO₄, and concentrated in vacuo. The residue was purified by column chromatography on silica gel, eluting with 5-30% EtOAc in PE to give (3-cyclopropyl-8-iodoquinolin-6-yl)methanol (500 mg, 91%) as a yellow solid. ESI-MS [M+H]⁺: 326.1.

Synthesis of 1-(3-cyclopropyl-6-(hydroxymethyl)quinolin-8-yl)-3-methylimidazolidine-2,4-dione. A solution of (3-cyclopropyl-8-iodoquinolin-6-yl)methanol (500 mg, 1.53 mmol), 3-methylimidazolidine-2,4-dione (526 mg, 4.61 mmol), Pd₂(dba)₃ (275 mg, 0.30 mmol), Xantphos (356 mg, 0.61 mmol) and Cs₂CO₃ (1.5 g, 4.60 mmol) in 1,4-dioxane (25 mL) was stirred at 65° C. for 8 h under N₂ atmosphere. The mixture was cooled to room temperature. The resulting mixture was filtered through a pad of Celite® and the filter cake was washed with DCM/MeOH (V/V=10/1, 100 mL). The filtrate was concentrated in vacuo. The residue was purified by column chromatography on silica gel, eluting with 0-5% MeOH in DCM to give 1-(3-cyclopropyl-6-(hydroxymethyl)quinolin-8-yl)-3-methylimidazolidine-2,4-dione (300 mg, 61%) as a yellow solid. ESI-MS [M+H]⁺:321.1.

Synthesis of 1-(6-(chloromethyl)-3-cyclopropylquinolin-8-yl)-3-methylimidazolidine-2,4-dione. To a solution of 1-(3-cyclopropyl-6-(hydroxymethyl)quinolin-8-yl)-3-methylimidazolidine-2,4-dione (300 mg, 0.96 mmol) in DCM (9 mL) was added SOCl₂ (0.5 mL). The mixture was stirred at 25° C. for 2 h under N₂ atmosphere then concentrated in vacuo to give 1-(6-(chloromethyl)-3-cyclopropylquinolin-8-yl)-3-methylimidazolidine-2,4-dione (300 mg, 95%) as a yellow solid. ESI-MS [M+H]⁺: 330.1.

Synthesis of: 1-(6-(azidomethyl)-3-cyclopropylquinolin-8-yl)-3-methylimidazolidine-2,4-dione. To a mixture of 1-(6-(chloromethyl)-3-cyclopropylquinolin-8-yl)-3-methylimidazolidine-2,4-dione (300 mg, 0.91 mmol) in DMF (7 mL) was added NaN₃ (177 mg, 2.73 mmol). After stirring for 3 h at 25° C., water (50 mL) was added and mixture was extracted with EtOAc (80 mL×3). The combined organic layers were washed with brine (30 mL), dried over anhydrous Na₂SO₄, and concentrated in vacuo. The residue was purified by column chromatography on silica gel, eluting with 10-50% EtOAc in PE to give 1-(6-(azidomethyl)-3-cyclopropylquinolin-8-yl)-3-methylimidazolidine-2,4-dione (250 mg, 82%) as a yellow solid. ESI-MS [M+H]⁺: 337.1

Synthesis of 1-(6-(aminomethyl)-3-cyclopropylquinolin-8-yl)-3-methylimidazolidine-2,4-dione. A solution of 1-(6-(azidomethyl)-3-cyclopropylquinolin-8-yl)-3-methylimidazolidine-2,4-dione (250 mg, 0.74 mmol) and 10% Pd/C (50 mg) in MeOH (10 mL) was stirred at 25° C. for 1 h under and atmosphere of H₂. The mixture was filtered by a pad of Celite®, and the filter cake was washed with DCM/MeOH (V/V=10/1, 50 mL). The filtrate was concentrated in vacuo. The residue was purified by column chromatography on silica gel, eluting with 0˜5% MeOH in DCM to give 1-(6-(aminomethyl)-3-cyclopropylquinolin-8-yl)-3-methylimidazolidine-2,4-dione (170 mg, 74%) as a yellow solid. ESI-MS [M+H]⁺: 311.1.

Synthesis of (1S,2S)—N-(6-(((3-cyclopropyl-8-(3-methyl-2,4-dioxoimidazolidin-1-yl)quinolin-6-yl)methyl)amino)pyrimidin-4-yl)-2-(4-methylpyrimidin-2-yl)cyclopropane-1-carboxamide. A solution of 1-(6-(aminomethyl)-3-cyclopropylquinolin-8-yl)-3-methylimidazolidine-2,4-dione (95 mg, 0.30 mmol), (1S,2S)—N-(6-chloropyrimidin-4-yl)-2-(4-methylpyrimidin-2-yl)cyclopropane-1-carboxamide (96 mg, 0.31 mmol) and DIPEA (198 mg, 1.53 mmol) in i-PrOH (3 mL) was stirred in a sealed tube. After degassing with N₂ for 1 min, the reaction was irradiated in a microwave reactor at 140° C. for 3.5 h. The mixture was cooled to room temperature and concentrated in vacuo. The residue was purified by preparative TLC, eluting with 10% MeOH in DCM to give (1S,2S)—N-(6-(((3-cyclopropyl-8-(3-methyl-2,4-dioxoimidazolidin-1-yl)quinolin-6-yl)methyl)amino)pyrimidin-4-yl)-2-(4-methylpyrimidin-2-yl)cyclopropane-1-carboxamide (41 mg, 24%) as a white solid. ESI-MS [M+H]⁺: 564.2. ¹H NMR (400 MHz, DMSO) δ=10.71 (s, 1H), 8.73 (d, J=2.2, 1H), 8.52 (d, J=5.1, 1H), 8.18 (s, 1H), 8.10 (s, 1H), 8.02 (d, J=2.2, 1H), 7.72 (s, 2H), 7.33 (s, 1H), 7.21 (d, J=5.1, 1H), 4.70 (s, 2H), 4.65 (s, 2H), 2.99 (s, 3H), 2.64-2.61 (m, 1H), 2.57-2.53 (m, 1H), 2.41 (s, 3H), 2.16-2.10 (m, 1H), 1.54-1.50 (m, 2H), 1.10-1.05 (m, 2H), 0.91-0.86 (m, 2H).

### Example 99

### Synthesis of (S,2S)—N-(6-(((5-cyclopropyl-7-(3-methyl-2,4-dioxoimidazolidin-1-yl)pyrazolo[1,5-a]pyridin-2-yl)methyl)amino)-2-methylpyrimidin-4-yl)-2-(4-methylpyrimidin-2-yl)cyclopropane-1-carboxamide (I-99)

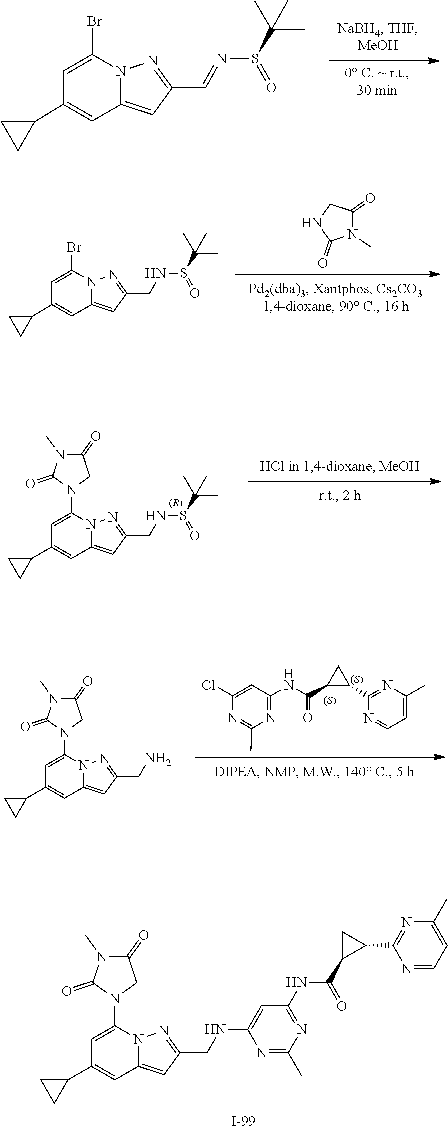

Synthesis of (R)—N-((7-bromo-5-cyclopropylpyrazolo[1,5-a]pyridin-2-yl)methyl)-2-methylpropane-2-sulfinamide. To a stirred solution of (R,E)-N-((7-bromo-5-cyclopropylpyrazolo[1,5-a]pyridin-2-yl)methylene)-2-methylpropane-2-sulfinamide (0.40 g, 1.1 mmol) in THF (6.0 mL) and MeOH (2.0 mL) was added NaBH₄ (83 mg, 2.2 mmol) portionwise at 0° C. The mixture was stirred at room temperature for 30 min. The reaction mixture was then quenched with NH₄Cl (sat, aq. 30 mL) and extracted with EtOAc (40 mL×2). The combined organics were washed with brine (80 mL), dried over anhydrous Na₂SO₄ and then concentrated in vacuo. The residue was purified by column chromatography on silica gel, eluting with 50-70% EtOAc in PE to give (R)—N-((7-bromo-5-cyclopropylpyrazolo[1,5-a]pyridin-2-yl)methyl)-2-methylpropane-2-sulfinamide (0.37 g, 91%) as a yellow solid. ESI-MS [M+H]⁺: 370.1.

Synthesis of (R)—N-((5-cyclopropyl-7-(3-methyl-2,4-dioxoimidazolidin-1-yl)pyrazolo[1,5-a]pyridin-2-yl)methyl)-2-methylpropane-2-sulfinamide. A solution of (R)—N-((7-bromo-5-cyclopropylpyrazolo[1,5-a]pyridin-2-yl)methyl)-2-methylpropane-2-sulfinamide (0.37 g, 1.1 mmol), 3-methylimidazolidine-2,4-dione (0.38 g, 3.3 mmol), Pd₂(dba)₃ (0.10 g, 0.11 mmol), Xantphos (0.13 g, 0.22 mmol) and Cs₂CO₃ (1.1 g, 3.3 mmol) in 1,4-dioxane (15 mL) was stirred at 90° C. for 16 h under N₂. The mixture was cooled to room temperature. The reaction mixture was filtered through a pad of Celite® and the filter cake was washed with DCM/MeOH (V/V=10/1, 50 mL). The filtrate was concentrated in vacuo. The residue was purified by column chromatography on silica gel, eluting with 20˜50% EtOAc in PE to give (R)—N-((5-cyclopropyl-7-(3-methyl-2,4-dioxoimidazolidin-1-yl)pyrazolo[1,5-a]pyridin-2-yl)methyl)-2-methylpropane-2-sulfinamide (0.32 g, 72%) as a yellow solid. ESI-MS [M+H]⁺: 404.2.

Synthesis of 1-(2-(aminomethyl)-5-cyclopropylpyrazolo[1,5-a]pyridin-7-yl)-3-methylimidazolidine-2,4-dione. To a solution of (R)—N-((5-cyclopropyl-7-(3-methyl-2,4-dioxoimidazolidin-1-yl)pyrazolo[1,5-a]pyridin-2-yl)methyl)-2-methylpropane-2-sulfinamide (0.32 g, 0.79 mmol) in MeOH (4.0 mL) was added HCl (4 M solution in 1, 4-dioxane, 5.0 mL). The resulting mixture was stirred at room temperature for 2 h. The reaction mixture was concentrated in vacuo and added NH₃ (7 N in MeOH, 5.0 mL). The mixture was stirred for another 10 min and then concentrated in vacuo. The residue was purified by preparative TLC, eluting with 10% MeOH in DCM to give 1-(2-(aminomethyl)-5-cyclopropylpyrazolo[1,5-a]pyridin-7-yl)-3-methylimidazolidine-2,4-dione (0.15 g, 63%) as a yellow solid. ESI-MS [M+H]⁺: 300.1.

Synthesis of (1S,2S)—N-(6-(((5-cyclopropyl-7-(3-methyl-2,4-dioxoimidazolidin-1-yl)pyrazolo[1,5-a]pyridin-2-yl)methyl)amino)-2-methylpyrimidin-4-yl)-2-(4-methylpyrimidin-2-yl)cyclopropane-1-carboxamide. A solution of 1-(2-(aminomethyl)-5-cyclopropylpyrazolo[1,5-a]pyridin-7-yl)-3-methylimidazolidine-2,4-dione (49 mg, 0.16 mmol), (1S,2S)—N-(6-chloro-2-methylpyrimidin-4-yl)-2-(4-methylpyrimidin-2-yl)cyclopropane-1-carboxamide (50 mg, 0.17 mmol) and DIPEA (0.22 g, 1.7 mmol) in NMP (3.0 mL) was stirred in a sealed tube. After degassing with N₂ for 1 min, the reaction was irradiated in a microwave reactor at 140° C. for 6 h. The mixture was cooled to room temperature. The reaction mixture was poured into water (20 mL) and extracted with EtOAc (20 ml×3). The combined organics were washed with water (50 mL×3) and brine (50 mL), dried over Na₂SO₄, and concentrated in vacuo. The residue was purified by column chromatography on silica gel, eluting with 0-5% MeOH in DCM to give (1S,2S)—N-(6-(((5-cyclopropyl-7-(3-methyl-2,4-dioxoimidazolidin-1-yl)pyrazolo[1,5-a]pyridin-2-yl)methyl)amino)-2-methylpyrimidin-4-yl)-2-(4-methylpyrimidin-2-yl)cyclopropane-1-carboxamide (45 mg, 49%) as a white solid. ESI-MS [M+H]⁺: 567.2. ¹H NMR (400 MHz, DMSO) δ 10.68 (s, 1H), 8.52 (d, J=5.1 Hz, 1H), 7.76 (t, J=5.9 Hz, 1H), 7.36 (d, J=1.8 Hz, 1H), 7.20 (d, J=5.1 Hz, 1H), 7.13 (s, 1H), 6.75 (d, J=1.8 Hz, 1H), 6.43 (s, 1H), 4.72-4.54 (m, 4H), 3.01 (s, 3H), 2.60-2.50 (m, 2H), 2.41 (s, 3H), 2.26 (s, 3H), 2.03-1.97 (m, 1H), 1.54-1.45 (m, 2H), 1.06-0.98 (m, 2H), 0.78-0.71 (m, 2H).

### Example 100

### Synthesis of (1S,2S)—N-(5-((5-cyclopropyl-7-(3-methyl-2,4-dioxoimidazolidin-1-yl)pyrazolo[1,5-a]pyridin-2-yl)methoxy)pyridazin-3-yl)-2-(4-methylpyrimidin-2-yl)cyclopropane-1-carboxamide (I-100)

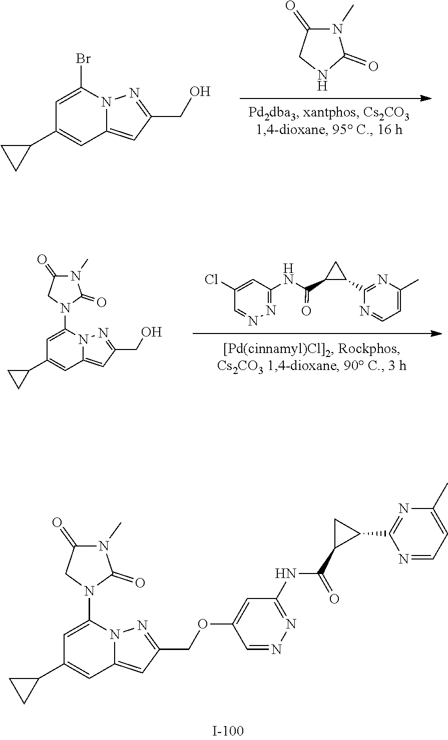

Synthesis of 1-(5-cyclopropyl-2-(hydroxymethyl)pyrazolo[1,5-a]pyridin-7-yl)-3-methylimidazolidine-2,4-dione. A solution of (7-bromo-5-cyclopropylpyrazolo[1,5-a]pyridin-2-yl)methanol (1.0 g, 3.8 mmol), 3-methylimidazolidine-2,4-dione (1.7 g, 15 mmol), Pd₂(dba)₃ (0.69 g, 0.76 mmol), Xantphos (0.87 g, 1.5 mmol) and Cs₂CO₃ (3.7 g, 11.4 mmol) in 1,4-dioxane (30 mL) was stirred at 95° C. for 16 h under N₂. The mixture was cooled to room temperature, filtered through Celite®, and the filter cake was washed by DCM (90 mL). The filtrate was concentrated in vacuo. The residue was purified by column chromatography on silica gel, eluting with 10-60% EtOAc in PE to give 1-(5-cyclopropyl-2-(hydroxymethyl)pyrazolo[1,5-a]pyridin-7-yl)-3-methylimidazolidine-2,4-dione (0.92 g, 82%) as a yellow solid. ESI-MS: [M+H]⁺, 301.1.

Synthesis of (1S,2S)—N-(5-((5-cyclopropyl-7-(3-methyl-2,4-dioxoimidazolidin-1-yl)pyrazolo[1,5-a]pyridin-2-yl)methoxy)pyridazin-3-yl)-2-(4-methylpyrimidin-2-yl)cyclopropane-1-carboxamide. A solution of 1-(5-cyclopropyl-2-(hydroxymethyl)pyrazolo[1,5-a]pyridin-7-yl)-3-methylimidazolidine-2,4-dione (0.20 g, 0.67 mmol), (1S,2S)—N-(5-chloropyridazin-3-yl)-2-(4-methylpyrimidin-2-yl)cyclopropane-1-carboxamide (0.19 g, 0.67 mmol), [Pd(cinnamyl)C1]2 (35 mg, 0.067 mmol), Rockphos (61 mg, 0.13 mmol) and Cs₂CO₃ (0.65 g, 2.0 mmol) in 1,4-dioxane (20 mL) was stirred at 90° C. for 3 h. The mixture was cooled to room temperature. The resulting mixture was filtered by Celite® and the filter cake was washed with DCM (80 mL). The organic layer was concentrated in vacuo. The residue was purified by column chromatography on silica gel, eluting with 0˜5% MeOH in DCM to give (1S,2S)—N-(5-((5-cyclopropyl-7-(3-methyl-2,4-dioxoimidazolidin-1-yl)pyrazolo[1,5-a]pyridin-2-yl)methoxy)pyridazin-3-yl)-2-(4-methylpyrimidin-2-yl)cyclopropane-1-carboxamide (50 mg, 13%) as a white solid. ESI-MS: [M+H]⁺, 554.0, ¹H NMR (400 MHz, DMSO) δ 11.40 (s, 1H), 8.78 (s, 1H), 8.54 (d, J=4.0 Hz, 1H), 8.14 (s, 1H), 7.44 (s, 1H), 7.22 (d, J=3.5 Hz, 1H), 6.84 (s, 1H), 6.69 (s, 1H), 5.45 (s, 2H), 4.69 (s, 2H), 3.02 (s, 3H), 2.69-2.68 (m, 1H), 2.58-2.56 (m, 1H), 2.43 (s, 3H), 2.04-1.99 (m, 1H), 1.63-1.52 (m, 2H), 1.09-1.00 (m, 2H), 0.82-0.72 (m, 2H).

### Example 101

### Synthesis of (S,2S)—N-(6-(((5-cyclopropyl-7-(3-methyl-2,4-dioxoimidazolidin-1-yl)pyrazolo[1,5-a]pyridin-2-yl)methyl)amino)pyrimidin-4-yl)-2-(4-methylpyrimidin-2-yl)cyclopropane-1-carboxamide (I-101)

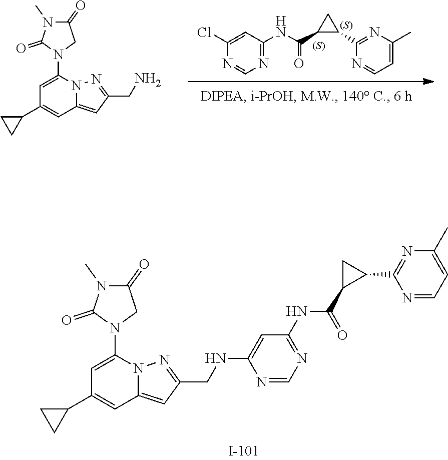

A solution of 1-(2-(aminomethyl)-5-cyclopropylpyrazolo[1,5-a]pyridin-7-yl)-3-methylimidazolidine-2,4-dione (50 mg, 0.17 mmol), (1S,2S)—N-(6-chloropyrimidin-4-yl)-2-(4-methylpyrimidin-2-yl)cyclopropane-1-carboxamide (49 mg, 0.17 mmol) and DIPEA (0.22 g, 1.7 mmol) in i-PrOH (3 mL) was stirred in a sealed tube. After degassing with N₂ for 1 min, the reaction mixture was irradiated in a microwave reactor at 140° C. for 6 h. The mixture was cooled to room temperature and concentrated in vacuo. The residue was purified by preparative TLC, eluting with 5% MeOH in DCM to give (1S,2S)—N-(6-(((5-cyclopropyl-7-(3-methyl-2,4-dioxoimidazolidin-1-yl)pyrazolo[1,5-a]pyridin-2-yl)methyl)amino)pyrimidin-4-yl)-2-(4-methylpyrimidin-2-yl)cyclopropane-1-carboxamide (50 mg, 54%) as a white solid. ESI-MS [M+H]⁺: 553.3, ¹H NMR (400 MHz, DMSO) δ 10.68 (s, 1H), 8.52 (d, J=5.1 Hz, 1H), 8.19 (s, 1H), 7.90 (s, 1H), 7.36 (d, J=1.8 Hz, 1H), 7.30 (s, 1H), 7.21 (d, J=5.1 Hz, 1H), 6.75 (d, J=1.8 Hz, 1H), 6.42 (s, 1H), 4.67-4.49 (m, 4H), 3.00 (s, 3H), 2.65-2.57 (m, 1H), 2.54-2.50 (m, 1H), 2.41 (s, 3H), 2.03-1.97 (m, 1H), 1.55-1.47 (m, 2H), 1.04-1.00 (m, 2H), 0.76-0.72 (m, 2H).

### Example 102

### Synthesis of (S,2S)—N-(5-(((R^{•})-1-(5-cyclopropyl-7-(3-methyl-2,4-dioxoimidazolidin-1-yl)pyrazolo[1,5-a]pyridin-2-yl)ethyl)amino)-1,2,4-triazin-3-yl)-2-(4-methylpyrimidin-2-yl)cyclopropane-1-carboxamide (I-102)

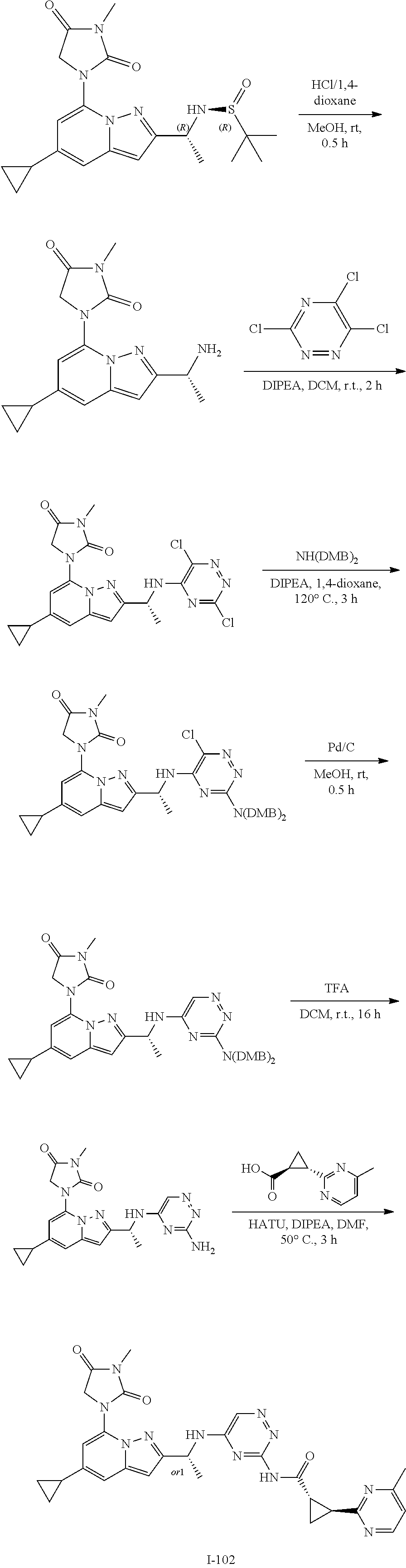

Synthesis of (R^{•})-1-(2-(1-aminoethyl)-5-cyclopropylpyrazolo[1,5-a]pyridin-7-yl)-3-methylimidazolidine-2,4-dione, isomer 1. To a mixture of (R)—N—((R^{•})-1-(5-cyclopropyl-7-(3-methyl-2,4-dioxoimidazolidin-1-yl)pyrazolo[1,5-a]pyridin-2-yl)ethyl)-2-methylpropane-2-sulfinamide, isomer 1 (0.32 g, 0.77 mmol) in MeOH (5 mL) was added HCl (4 M solution in 1,4-dioxane, 5 mL) at room temperature. The resulting reaction mixture was stirred at room temperature for 0.5 h then concentrated in vacuo. The residue was neutralized with NH₃ (7 M solution in MeOH, 10 mL) and concentrated in vacuo. The residue was purified by column chromatography on silica gel eluting with a gradient of 0-10% MeOH in DCM to afford (R^{•})-1-(2-(1-aminoethyl)-5-cyclopropylpyrazolo[1,5-a]pyridin-7-yl)-3-methylimidazolidine-2,4-dione, isomer 1 (200 mg, 83%) as a yellow oil. ESI-MS [M+H]⁺: 314.2.

Synthesis of (R^{•})-1-(5-cyclopropyl-2-(1-((3,6-dichloro-1,2,4-triazin-5-yl)amino)ethyl)pyrazolo[1,5-a]pyridin -7-yl)-3-methylimidazolidine-2,4-dione. To a mixture of (R^{•})-1-(2-(1-aminoethyl)-5-cyclopropylpyrazolo[1,5-a]pyridin-7-yl)-3-methylimidazolidine-2,4-dione, isomer 1 (170 mg, 0.54 mmol), 3,5,6-trichloro-1,2,4-triazine (220 mg, 1.1 mmol) in DCM (10 mL) was added DIPEA (206 mg, 1.6 mmol). The reaction mixture was stirred at room temperature for 2 h. The reaction mixture was quenched by H₂O (25 mL) and extracted by DCM (25 mL×3). The combined organic layers were washed with brine (30 mL), dried over anhydrous Na₂SO₄ and concentrated in vacuo to give the crude, which was purified by column chromatography on silica gel eluting with a gradient of 0-100% EA in PE to afford (R^{•})-1-(5-cyclopropyl-2-(1-((3,6-dichloro-1,2,4-triazin-5-yl)amino)ethyl)pyrazolo[1,5-a]pyridin-7-yl)-3-meth ylimidazolidine-2,4-dione (0.14 g, 56%) as yellow oil. ESI-MS [M+H]⁺: 461.1.

Synthesis of (R^{•})-1-(2-(1-((3-(bis(3,5-dimethoxybenzyl)amino)-6-chloro-1,2,4-triazin-5-yl)amino)ethyl)-5-cyclopropylpyrazolo[1,5-a]pyridin-7-yl)-3-methylimidazolidine-2,4-dione. A mixture of (R^{•})-1-(5-cyclopropyl-2-(1-((3,6-dichloro-1,2,4-triazin-5-yl)amino)ethyl)pyrazolo[1,5-a]pyridin-7-yl)-3-methylimidazolidine-2,4-dione (110 mg, 0.24 mmol), NH(DMB)₂ (152 mg, 0.48 mmol) and DIEA (93 mg, 0.72 mmol) in dioxane (2.0 mL) was stirred in a sealed tube. After degassing with N₂ for 1 min, the reaction was irradiated in a microwave reactor at 120° C. for 3 h. After cooling to room temperature, the reaction was concentrated in vacuo to give the crude product, and the residue was purified by column chromatography on silica gel eluting with a gradient of 0-80% EtOAc in PE to afford (R^{•})-1-(2-(1-((3-(bis(3,5-dimethoxybenzyl)amino)-6-chloro-1,2,4-triazin-5-yl)amino)ethyl)-5-cyclopropylpyrazolo[1,5-a]pyridin-7-yl)-3-methylimidazolidine-2,4-dione (125 mg, 70%) as yellow oil. ESI-MS [M+H]⁺:742.2.

Synthesis of (R^{•})-1-(2-(1-((3-(bis(3,5-dimethoxybenzyl)amino)-1,2,4-triazin-5-yl)amino)ethyl)-5-cyclopropylpyrazolo[1,5-a]pyridin-7-yl)-3-methylimidazolidine-2,4-dione. A mixture of (R^{•})-1-(2-(1-((3-(bis(3,5-dimethoxybenzyl)amino)-6-chloro-1,2,4-triazin-5-yl)amino)ethyl)-5-cyclopropyl pyrazolo[1,5-a]pyridin-7-yl)-3-methylimidazolidine-2,4-dione (120 mg, 0.16 mmol) and Pd/C (40 mg) in MeOH was stirred at room temperature for 0.5 h. The reaction mixture was filtered through Celite® and the filter cake was washed with MeOH (30 mL). The filtrate was concentrated in vacuo to afford (R^{•})-1-(2-(1-((3-(bis(3,5-dimethoxybenzyl)amino)-1,2,4-triazin-5-yl)amino)ethyl)-5-cyclopropylpyrazolo[1,5-a]pyridin-7-yl)-3-methylimidazolidine-2,4-dione (84 mg, 74%) as yellow oil. ESI-MS [M+H]⁺:708.3.

Synthesis of (R^{•})-1-(2-(1-((3-amino-1,2,4-triazin-5yl)amino)ethyl)-5-cyclopropylpyrazolo[1,5-a]pyridin-7-yl)-3-methylimidazolidine-2,4-dione. To a mixture of (R^{•})-1-(2-(1-((3-(bis(3,5-dimethoxybenzyl)amino)-1,2,4-triazin-5-yl)amino)ethyl)-5-cyclopropylpyrazolo[1,5-a]pyridin-7-yl)-3-methylimidazolidine-2,4-dione (84 mg, 0.12 mmol) in DCM (6.0 mL) was added TFA (3.0 mL). The mixture was stirred at room temperature for 16 h then diluted with NaHCO₃ (sat. aq., 15 mL) and extracted with EtOAc (20 mL×3). The combined organic layers were washed with brine (30 mL), dried over anhydrous Na₂SO₄, and concentrated in vacuo. The residue was purified by column chromatography on silica gel eluting with a gradient of 0-20% MeOH in DCM to afford (R^{•})-1-(2-(1-((3-amino-1,2,4-triazin-5-yl)amino)ethyl)-5-cyclopropylpyrazolo[1,5-a]pyridin-7-yl)-3-methylimidazolidine-2,4-dione (35 mg, 71%) as a yellow solid. ESI-MS [M+H]⁺: 408.2.

Synthesis of (S,2S)—N-(5-(((R^{•})-1-(5-cyclopropyl-7-(3-methyl-2,4-dioxoimidazolidin-1-yl)pyrazolo[1,5-a]pyridin-2-yl)ethyl)amino)-1,2,4-triazin-3-yl)-2-(4-methylpyrimidin-2-yl)cyclopropane-1-carboxamide. To the mixture of (R^{•})-1-(2-(1-((3-amino-1,2,4-triazin-5-yl)amino)ethyl)-5-cyclopropylpyrazolo[1,5-a]pyridin-7-yl)-3-methylimidazolidine-2,4-dione (35 mg, 0.086 mmol), (1S,2S)-2-(4-methylpyrimidin-2-yl)cyclopropane-1-carboxylic acid (22 mg, 0.12 mmol), HATU (61 mg, 0.16 mmol) in DMF (3 mL) was added DIPEA (52 mg, 0.405 mmol) at room temperature. The reaction mixture was stirred at 50° C. for 3 h. After cooling to room temperature, the reaction mixture was diluted with H₂O (25 mL) and extracted with EtOAc (20 mL×3). The combined organic layers were washed with brine (20 mL), dried over anhydrous Na₂SO₄ and concentrated in vacuo to give the crude product, which was purified by preparative HPLC to afford (1S,2S)—N-(5-(((R^{•})-1-(5-cyclopropyl-7-(3-methyl-2,4-dioxoimidazolidin-1-yl)pyrazolo[1,5-a]pyridin-2-yl)ethyl)amino)-1,2,4-triazin-3-yl)-2-(4-methylpyrimidin-2-yl)cyclopropane-1-carboxamide (11 mg, 22%) as a white solid. ESI-MS [M+H]⁺:568.3. ¹H NMR (400 MHz, DMSO) δ 10.61 (s, 1H), 8.59 (d, J=8.1 Hz, 1H), 8.47 (d, J=5.1 Hz, 1H), 8.31 (s, 1H), 7.35 (d, J=1.4 Hz, 1H), 7.14 (d, J=5.0 Hz, 1H), 6.76 (d, J=1.6 Hz, 1H), 6.48 (s, 1H), 5.35-5.31 (m, 1H), 4.66 (s, 2H), 3.00 (s, 3H), 2.56-2.54 (m, 2H), 2.37 (s, 3H), 2.03-1.99 (m, 1H), 1.56-1.51 (m, 5H), 1.05-1.00 (m, 2H), 0.76-0.72 (m, 2H).

### Example 103

### Synthesis of (S,2S)—N-(6-(((R^{•})-1-(5-cyclopropyl-7-(2-oxo-3-azabicyclo[3.1.0]hexan-3-yl)pyrazolo[1,5-a]pyridin-2-yl)ethyl)amino)pyrimidin-4-yl)-2-(4-methylpyrimidin-2-yl)cyclopropane-1-carboxamide (I-103)

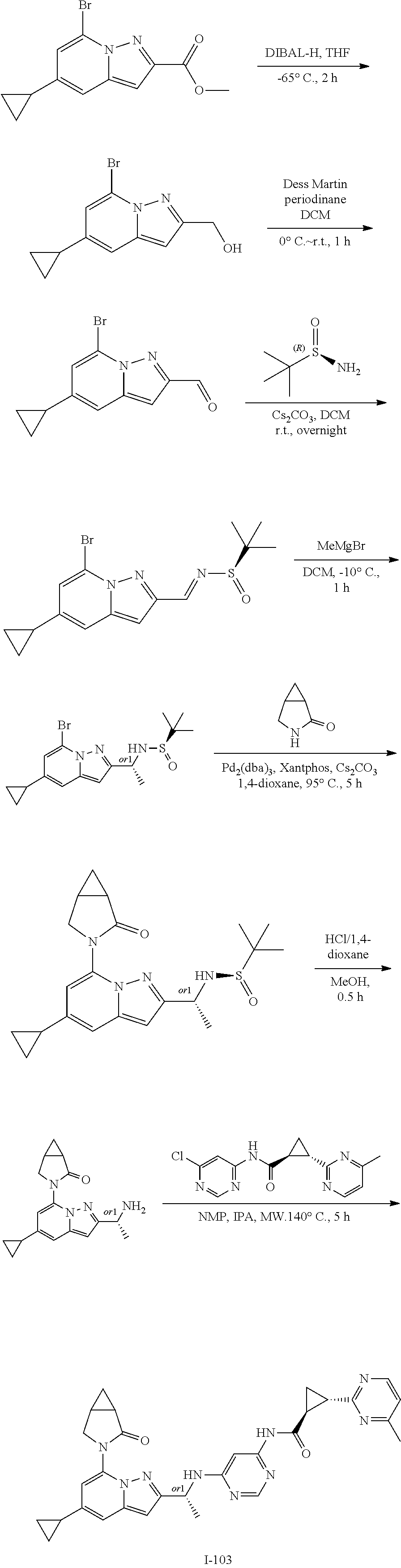

Synthesis of (7-bromo-5-cyclopropylpyrazolo[1,5-a]pyridin-2-yl)methanol. To a solution of methyl 7-bromo-5-cyclopropylpyrazolo[1,5-a]pyridine-2-carboxylate (760 mg, 2.6 mmol) in THF (20 mL) was added DIBAL-H (7.3 mL, 7.3 mmol, 1M solution in THF) at −65° C. The reaction mixture was stirred at −65° C. for 2 h then quenched with NH₄Cl (sat. aq., 30 mL) and extracted with EtOAc (40 mL×3). The combined organic layers were washed with brine (40 mL), dried over anhydrous Na₂SO₄, and concentrated in vacuo to give the crude. The residue was purified by column chromatography on silica gel eluting with a gradient of 0-80% EtOAc in PE to afford (7-bromo-5-cyclopropylpyrazolo[1,5-a]pyridin-2-yl)methanol (650 mg, 94%) as a yellow solid. ESI-MS [M+H]⁺:267.1.

Synthesis of 7-bromo-5-cyclopropylpyrazolo[1,5-a]pyridine-2-carbaldehyde. To the mixture of (7-bromo-5-cyclopropylpyrazolo[1,5-a]pyridin-2-yl)methanol (630 mg, 2.4 mmol) in DCM (30 mL) was added Dess Martin periodinane (2.5 g, 5.9 mmol) at 0° C. The reaction mixture was stirred at room temperature for 1 h then diluted with DCM (30 mL) and washed with NaHCO₃ (sat. aq., 30 mL) followed by Na₂S₂O₃ (sat. aq., 30 mL). The organic layer was dried over anhydrous Na₂SO₄ and concentrated in vacuo. The residue was purified by column chromatography on silica gel eluting with a gradient of 0-50% EtOAc in PE to afford 7-bromo-5-cyclopropylpyrazolo[1,5-a]pyridine-2-carbaldehyde (580 mg, 91%) as a yellow solid. ESI-MS [M+H]⁺:265.1.

Synthesis of (R,E)-N-((7-bromo-5-cyclopropylpyrazolo[1,5-a]pyridin-2-yl)methylene)-2-methylpropane-2-sulfinamide. A mixture of 7-bromo-5-cyclopropylpyrazolo[1,5-a]pyridine-2-carbaldehyde (580 mg, 2.2 mmol), (R)-2-methylpropane-2-sulfinamide (399 mg, 3.3 mmol) and Cs₂CO₃ (1.4 g, 4.4 mmol) in DCM (20 mL) was stirred at room temperature overnight. The reaction mixture was diluted with H₂O (30 mL) and extracted by DCM (30 mL×3). The organic layers were washed with brine (40 mL), dried over anhydrous Na₂SO₄, concentrated in vacuo. The residue was purified by column chromatography on silica gel eluting with 0-100% EtOAc in PE to afford (R,E)-N-((7-bromo-5-cyclopropylpyrazolo[1,5-a]pyridin-2-yl)methylene)-2-methylpropane-2-sulfinamide (0.80 g, 99%) as a yellow solid. ESI-MS [M+H]⁺:368.1.

Synthesis of (R)—N—((R^{•})-1-(7-bromo-5-cyclopropylpyrazolo[1,5-a]pyridin-2-yl)ethyl)-2-methylpropane-2-sulfinamide. To the mixture of (R,E)-N-((7-bromo-5-cyclopropylpyrazolo[1,5-a]pyridin-2-yl)methylene)-2-methylpropane-2-sulfinamide (800 mg, 2.2 mmol) in THF (40 mL) was added CH₃MgBr (2.2 mL, 6.6 mmol, 3 M solution in diethyl ether) at −10° C. The reaction mixture was stirred at −10° C. for 1 h. The reaction mixture was quenched by NH₄Cl (sat. aq. 30 mL) at -10° C., and extracted with EtOAc (50 mL×3). The combined organic layers were washed with brine (40 mL), dried over anhydrous Na₂SO₄ and concentrated in vacuo. The residue was purified by column chromatography on silica gel eluting with 0-80% a gradient of EtOAc in PE to afford (R)—N—((R^{•})-1-(7-bromo-5-cyclopropylpyrazolo[1,5-a]pyridin-2-yl)ethyl)-2-methylpropane-2-sulfinamide (460 mg, 54%) as a yellow solid. ESI-MS [M+H]⁺:384.1.

Synthesis of (R)—N—((R^{•})-1-(5-cyclopropyl-7-(2-oxo-3-azabicyclo[3.1.0]hexan-3-yl)pyrazolo[1,5-a]pyridin-2-yl)ethyl)-2-methylpropane-2-sulfinamide. To a mixture of (R)—N—((R^{•})-1-(7-bromo-5-cyclopropylpyrazolo[1,5-a]pyridin-2-yl)ethyl)-2-methylpropane-2-sulfinamide (100 mg, 0.26 mmol), 3-azabicyclo[3.1.0]hexan-2-one (50 mg, 0.52 mmol) and Cs₂CO₃ (254 mg, 0.78 mmol) in 1,4-dioxane (5 mL) was added Pd₂(dba)₃ (48 mg, 0.052 mmol) and Xantphos (58 mg, 0.10 mmol). The reaction mixture was stirred at 95° C. for 5 h under N₂. The reaction was cooled to room temperature then filtered through Celite®. The filter cake was washed with DCM/MeOH (10/1, 20 mL). The filtrate was concentrated in vacuo to give the crude, which was purified by column chromatography on silica gel eluting with a gradient of 0-80% EtOAc in PE to afford (R)—N-((1R^{•})-1-(5-cyclopropyl-7-(2-oxo-3-azabicyclo[3.1.0]hexan-3-yl)pyrazolo[1,5-a]pyridin-2-yl)ethyl)-2-methylpropane-2-sulfinamide (100 mg, 96%) as a yellow solid. ESI-MS [M+H]⁺:401.2.

Synthesis of 3-(2-((R^{•})-1-aminoethyl)-5-cyclopropylpyrazolo[1,5-a]pyridin-7-yl)-3-azabicyclo[3.1.0]hexan-2-one. To the mixture of (R)—N-((1R^{•})-1-(5-cyclopropyl-7-(2-oxo-3-azabicyclo[3.1.0]hexan-3-yl)pyrazolo[1,5-a]pyridin-2-yl)ethyl)-2-methylpropane-2-sulfinamide (100 mg, 0.25 mmol) in MeOH (5 mL) was added HCl (2 mL, 8 mmol, 4M solution in 1,4-dioxane). The reaction mixture was stirred at room temperature for 0.5 h then concentrated in vacuo. The residue was neutralized with NH₃ (10 mL, 7M solution in MeOH) and concentrated in vacuo to give the crude, which was purified by preparative TLC (eluent: MeOH/DCM=1/8) to afford 3-(2-((R^{•})-1-aminoethyl)-5-cyclopropylpyrazolo[1,5-a]pyridin-7-yl)-3-azabicyclo[3.1.0]hexan-2-one (60 mg, 81%) as a yellow solid. ESI-MS [M+H]⁺:297.2.

Synthesis of (S,2S)—N-(6-(((R^{•})-1-(5-cyclopropyl-7-(2-oxo-3-azabicyclo[3.1.0]hexan-3-yl)pyrazolo[1,5-a]pyridin-2-yl)ethyl)amino)pyrimidin-4-yl)-2-(4-methylpyrimidin-2-yl)cyclopropane-1-carboxamide. A mixture of 3-(2-((R^{•})-1-aminoethyl)-5-cyclopropylpyrazolo[1,5-a]pyridin-7-yl)-3-azabicyclo[3.1.0]hexan-2-one (60 mg, 0.2 mmol), (1S,2S)—N-(6-chloropyrimidin-4-yl)-2-(4-methylpyrimidin-2-yl)cyclopropane-1-carboxamide (59 mg, 0.2 mmol) and DIPEA (77 mg, 0.6 mmol) in iPrOH (2 mL) was sealed in a tube and irradiated in a microwave reactor at 140° C. for 5 h. The reaction was cooled to room temperature and concentrated in vacuo to give the crude, which was purified by preparative TLC (eluent: DCM/MeOH=10/1) to afford (1S,2S)—N-(6-(((1R^{•})-1-(5-cyclopropyl-7-(2-oxo-3-azabicyclo[3.1.0]hexan-3-yl)pyrazolo[1,5-a]pyridin-2-yl)ethyl)amino)pyrimidin-4-yl)-2-(4-methylpyrimidin-2-yl)cyclopropane-1-carboxamide (30 mg, 27%) as pale solid. ESI-MS [M+H]⁺:550.2. ¹H NMR (400 MHz, DMSO) δ 10.65 (s, 1H), 8.52 (d, J=5.1 Hz, 1H), 8.16 (s, 1H), 7.78 (s, 1H), 7.29-7.27 (m, 2H), 7.21 (d, J=5.1 Hz, 1H), 6.55-6.54 (m, 1H), 6.35 (d, J=2.6 Hz, 1H), 5.45 (s, 1H), 4.13-4.09 (m, 1H), 3.72-3.70 (m, 1H), 2.64-2.59 (m, 1H), 2.56-2.54 (m, 1H), 2.41 (s, 3H), 2.15-2.11 (m, 1H), 2.04-1.99 (m, 1H), 1.97-1.93 (m, 1H), 1.56-1.46 (m, 5H), 1.22-1.17 (m, 1H), 1.09-1.05 (m, 1H), 1.00-0.95 (m, 2H), 0.76-0.68 (m, 2H).

### Example 104

### Synthesis of (S,2S)—N-(6-(((R^{•})-1-(5-cyclopropyl-7-(3-methyl-2,4-dioxoimidazolidin-1-yl)pyrazolo[1,5-a]pyrimidin-2-yl)ethyl)amino)pyrimidin-4-yl)-2-(4-methylpyrimidin-2-yl)cyclopropane-1-carboxamide (I-104)

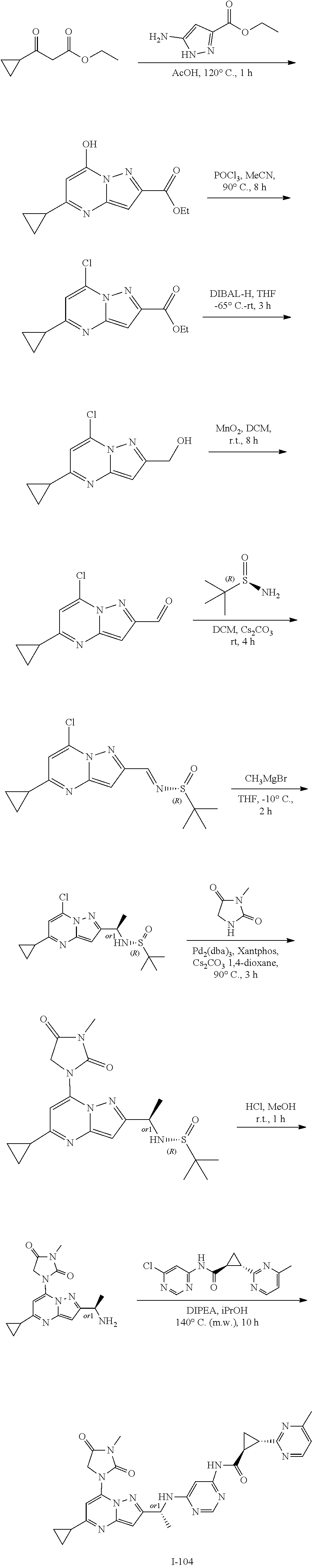

Synthesis of ethyl 5-cyclopropyl-7-hydroxypyrazolo[1,5-a]pyrimidine-2-carboxylate. A solution of ethyl 3-cyclopropyl-3-oxopropanoate (3 g, 19.2 mmol) and ethyl 5-amino-1H-pyrazole-3-carboxylate (3 g, 19.2 mmol) in AcOH (15 mL) was stirred at 120° C. for 1 h. The reaction mixture was filtered. The filtrant was dried in vacuo to give ethyl 5-cyclopropyl-7-hydroxypyrazolo[1,5-a]pyrimidine-2-carboxylate (1.5 g, 32%) as a yellow solid. ESI-MS [M+H]⁺: 248.2.

Synthesis of ethyl 7-chloro-5-cyclopropylpyrazolo[1,5-a]pyrimidine-2-carboxylate. A solution of ethyl 5-cyclopropyl-7-hydroxypyrazolo[1,5-a]pyrimidine-2-carboxylate (400 mg, 1.6 mmol) and POCl₃ (734 mg, 4.8 mmol) in MeCN (10 mL) was stirred at 90° C. for 8 h under nitrogen. The reaction was cooled to 0° C., quenched with ice water (50 mL) and NaHCO₃ (sat. aq., 50 mL) then extracted with EtOAc (50 mL×3). The combined organic layers were washed with brine (60 mL), dried over anhydrous Na₂SO₄ and concentrated in vacuo to give the crude, which was purified with silica gel eluting with a gradient of 0-50% EtOAc in PE to give ethyl 7-chloro-5-cyclopropylpyrazolo[1,5-a]pyrimidine-2-carboxylate (300 mg, 70%) as a yellow solid. ESI-MS [M+H]⁺: 266.2.

Synthesis of (7-chloro-5-cyclopropylpyrazolo[1,5-a]pyrimidin-2-yl)methanol. To a solution of ethyl 7-chloro-5-cyclopropylpyrazolo[1,5-a]pyrimidine-2-carboxylate (300 mg, 1.1 mmol) in dry THF (10 mL) at −65° C. was added DIBAL-H (3.4 mL, 3.4 mmol, 1M solution in hexane) under N₂, The mixture was stirred at −65° C. for 1 h then room temperature for 2 h. The reaction was quenched with NH₄Cl (sat. aq., 30 mL) and extracted with EtOAc (40 mL×3). The combined organic layers were washed with brine (40 mL), dried over anhydrous Na₂SO₄ and concentrated in vacuo to give the crude, which was purified with silica gel column chromatography eluting with a gradient of 0-7% MeOH in DCM to give the (7-chloro-5-cyclopropylpyrazolo[1,5-a]pyrimidin-2-yl)methanol as a yellow solid. (200 mg, 81%), ESI-MS [M+H]⁺: 224.2.

Synthesis of 7-chloro-5-cyclopropylpyrazolo[1,5-a]pyrimidine-2-carbaldehyde. A mixture of (7-chloro-5-cyclopropylpyrazolo[1,5-a]pyrimidin-2-yl)methanol (200 mg, 0.90 mmol) and MnO₂ (783 mg, 9.0 mmol) in DCM (10 mL) was stirred at room temperature for 8 h. The reaction mixture was filtered, and the filtrate was concentrated in vacuo to give the crude, which was purified with preparative TLC (eluent: DCM/MeOH=15/1) to give the 7-chloro-5-cyclopropyl pyrazolo[1,5-a]pyrimidine-2-carbaldehyde as a yellow solid. (160 mg, 80%), ESI-MS [M+H]⁺: 222.2.

Synthesis of (R,E)-N-((7-chloro-5-cyclopropylpyrazolo[1,5-a]pyrimidin-2-yl)methylene)-2-methylpropane-2-sulfinamide. A mixture of 7-chloro-5-cyclopropylpyrazolo[1,5-a]pyrimidine-2-carbaldehyde (150 mg, 0.68 mmol), (R)-2-methylpropane-2-sulfinamide (123 mg, 1.02 mmol) and Cs₂CO₃ (443 mg, 1.36 mmol) in DCM (20 mL) was stirred at room temperature for 4 h. The reaction was washed with H₂O (30 mL), extracted with DCM (30 mL×3). The combined organic layers were washed with brine (40 mL), dried over anhydrous Na₂SO₄, concentrated in vacuo to give the crude, which was purified with preparative TLC (eluent: DCM/MeOH=20/1) to give (R,E)-N-((7-chloro-5-cyclopropylpyrazolo[1,5-a]pyrimidin-2-yl)methylene)-2-methylpropane-2-sulfinamide as a yellow solid. (150 mg, 68%). ESI-MS [M+H]⁺: 325.2.

Synthesis of ((R^{•})-1-(7-chloro-5-cyclopropylpyrazolo[1,5-a]pyrimidin-2-yl)ethyl)-2-methylpropane-2-sulfinamide. To a solution of (R,E)-N-((7-chloro-5-cyclopropylpyrazolo[1,5-a]pyrimidin-2-yl)methylene)-2-methylpropane-2-sulfinamide (150 mg, 0.46 mmol) in THF (15 mL) was added CH₃MgBr (0.46 mL, 1.38 mmol, 3M solution in diethyl ether) at -10° C. for 2 h under nitrogen. The reaction was quenched with NH₄Cl (sat. aq., 30 mL) then extracted with EtOAc (30 mL×3). The combined organic layers were washed with brine (40 mL), dried over anhydrous Na₂SO₄ and concentrated in vacuo. The crude was purified by preparative TLC (eluent: DCM/MeOH=20/1) to give the (R)—N—((R^{•})-1-(7-chloro-5-cyclopropylpyrazolo[1,5-a]pyrimidin-2-yl)ethyl)-2-methylpropane-2-sulfinamide as a yellow solid. (120 mg, 77%). ESI-MS [M+H]⁺: 341.2.

Synthesis of (R)—N—((R^{•})-1-(5-cyclopropyl-7-(3-methyl-2,4-dioxoimidazolidin-1-yl)pyrazolo[1,5-a]pyrimidin-2-yl)ethyl)-2-methylpropane-2-sulfinamide. To a solution of (R)—N—((R^{•})-1-(7-chloro-5-cyclopropylpyrazolo[1,5-a]pyrimidin-2-yl)ethyl)-2-methylpropane-2-sulfinamide (120 mg, 0.35 mmol), 3-methylimidazolidine-2,4-dione (120 mg, 1.05 mmol) and Cs₂CO₃ (342 mg, 1.05 mmol) in 1,4-dioxane (10 mL) was added Pd₂(dba)₃ (32 mg, 0.035 mmol) and Xantphos (20 mg, 0.035 mmol). The reaction mixture was stirred at 90° C. for 3 h under nitrogen. The reaction was cooled to room temperature, diluted with H₂O (30 mL) then extracted with EtOAc (30 mL×3). The combined organic layers were washed with brine (40 mL), dried over anhydrous Na₂SO₄ and concentrated in vacuo to give the crude, which was purified with preparative TLC (eluent: DCM/MeOH=15/1) to give (R)—N—((R^{•})-1-(5-cyclopropyl-7-(3-methyl-2,4-dioxoimidazolidin-1-yl)pyrazolo[1,5-a]pyrimidin-2-yl)ethyl)-2-methylpropane-2-sulfinamide (90 mg, 62%) as a yellow solid. ESI-MS [M+H]⁺: 419.2.

Synthesis of (R^{•})-1-(2-(1-aminoethyl)-5-cyclopropylpyrazolo[1,5-a]pyrimidin-7-yl)-3-methylimidazolidine-2,4-dione. To a solution of (R)—N—((R^{•})-1-(5-cyclopropyl-7-(3-methyl-2,4-dioxoimidazolidin-1-yl)pyrazolo[1,5-a]pyrimidin-2-yl)ethyl)-2-methylpropane-2-sulfinamide (90 mg, 0.21 mmol) in MeOH (5 mL) was added HCl (5 mL, 4M solution in 1,4-dioxane). The resulting mixture was stirred at room temperature for 1 then concentrated in vacuo. The residue was neutralized with NH₃ (10 mL, 7 M solution in MeOH) and concentrated in vacuo to give the crude, which was purified with preparative TLC (eluent: DCM/MeOH=8/1) to give (R^{•})-1-(2-(1-aminoethyl)-5-cyclopropylpyrazolo[1,5-a]pyrimidin-7-yl)-3-methylimidazolidine-2,4-dione as the hydrochloric acid salt (45 mg, 68%) as a yellow solid. ESI-MS [M+H]⁺: 315.2.

Synthesis of (S,2S)—N-(6-(((R^{•})-1-(5-cyclopropyl-7-(3-methyl-2,4-dioxoimidazolidin-1-yl)pyrazolo[1,5-a]pyrimidin-2-yl)ethyl)amino)pyrimidin-4-yl)-2-(4-methylpyrimidin-2-yl)cyclopropane-1-carboxamide. A solution of (R^{•})-1-(2-(1-aminoethyl)-5-cyclopropylpyrazolo[1,5-a]pyrimidin-7-yl)-3-methylimidazolidine-2,4-dione (40 mg, 0.13 mmol), (1S,2S)—N-(6-chloropyrimidin-4-yl)-2-(4-methylpyrimidin-2-yl)cyclopropane-1-carboxamide (46 mg, 0.16 mmol) and DIPEA (50 mg, 0.39 mmol) in i-PrOH (3 mL) was sealed in a tube and irradiated in a microwave reactor at 140° C. for 10 h. The reaction was cooled to room temperature, concentrated in vacuo to give the crude product, which was purified by preparative TLC (eluent: DCM/MeOH=10/1) to give (1S,2S)—N-(6-(((R^{•})-1-(5-cyclopropyl-7-(3-methyl-2,4-dioxoimidazolidin-1-yl)pyrazolo[1,5-a]pyrimidin-2-yl)ethyl)amino)pyrimidin-4-yl)-2-(4-methylpyrimidin-2-yl)cyclopropane-1-carboxamide (30 mg, 41%) as a white solid. ESI-MS [M+H]⁺: 568.2. ¹H NMR (400 MHz, DMSO) δ 10.67 (s, 1H), 8.52 (d, J=5.1 Hz, 1H), 8.16 (s, 1H), 7.84 (s, 1H), 7.32 (s, 1H), 7.21 (d, J=5.6 Hz, 2H), 6.38 (s, 1H), 5.47 (s, 1H), 4.98 (s, 2H), 3.01 (s, 3H), 2.66-2.60 (m, 2H), 2.41 (s, 3H), 2.20-2.16 (m, 1H), 1.54-1.48 (m, 5H), 1.10-0.98 (m, 4H).

### Example 105

### Synthesis (1S,2S)—N-(5-(((1R^{•})-1-(5-cyclopropyl-7-(2-oxo-3-azabicyclo[3.1.0]hexan-3-yl)pyrazolo[1,5-a]pyridin-2-yl)ethyl)amino)-1,2,4-triazin-3-yl)-2-(4-methylpyrimidin-2-yl)cyclopropane-1-carboxamide (I-105)

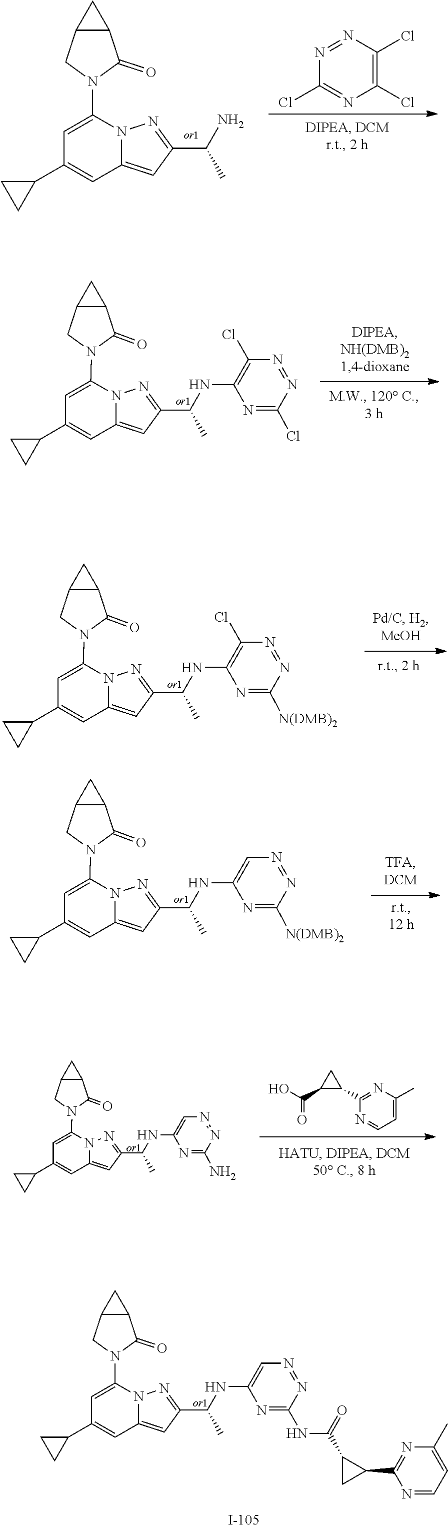

Synthesis of 3-(5-cyclopropyl-2-((R^{•})-1-((3,6-dichloro-1,2,4-triazin-5-yl)amino)ethyl)pyrazolo[1,5-a]pyridin-7-yl)-3-azabicyclo[3.1.0]hexan-2-one. A solution of 3-(2-((R^{•})-1-aminoethyl)-5-cyclopropylpyrazolo[1,5-a]pyridin-7-yl)-3-azabicyclo[3.1.0]hexan-2-one (260 mg, 0.88 mmol), DIPEA (341 mg, 2.64 mmol) and 3,5,6-trichloro-1,2,4-triazine (323 mg, 1.75 mmol) in DCM (10 mL) was stirred at room temperature for 2 h. The mixture was quenched with H₂O (20 mL) and then extracted with EtOAc (25 mL×3). The combined organics was washed with brine (20 mL), dried over Na₂SO₄, and concentrated in vacuo. The residue was purified by preparative TLC, eluting with 3% MeOH in DCM to give 3-(5-cyclopropyl-2-((R^{•})-1-((3,6-dichloro-1,2,4-triazin-5-yl)amino)ethyl)pyrazolo[1,5-a]pyridin-7-yl)-3-azabicyclo[3.1.0]hexan-2-one (270 mg, 69%) as a yellow solid. ESI-MS [M+H]⁺: 444.1.

Synthesis of 3-(2-((R^{•})-1-((3-(bis(3,4-dimethylbenzyl)amino)-6-chloro-1,2,4-triazin-5-yl)amino)ethyl)-5-cyclopropylpyrazolo[1,5-a]pyridin-7-yl)-3-azabicyclo[3.1.0]hexan-2-one. A solution of 3-(5-cyclopropyl-2-((R^{•})-1-((3,6-dichloro-1,2,4-triazin-5-yl)amino)ethyl)pyrazolo[1,5-a]pyridin-7-yl)-3-azabicyclo[3.1.0]hexan-2-one (270 mg, 0.61 mmol), NH(DMB)₂ (386 mg, 1.22 mmol) and DIPEA (236 mg, 1.83 mmol) in 1,4-dioxane (8 mL) was stirred in a sealed tube. After degassing with N₂ for 1 min, the reaction mixture was irradiated in a microwave reactor at 120° C. for 3 h. The mixture was cooled to room temperature and concentrated in vacuo. The residue was purified by prep-TLC, eluting with 5% MeOH in DCM to give 3-(2-((R^{•})-1-((3-(bis(3,4-dimethylbenzyl)amino)-6-chloro-1,2,4-triazin-5-yl)amino)ethyl)-5-cyclopropylpyrazolo[1,5-a]pyridin-7-yl)-3-azabicyclo[3.1.0]hexan-2-one (270 mg, 67%) as a yellow solid. ESI-MS [M+H]⁺: 661.3.

Synthesis of 3-(2-((R^{•})-1-((3-(bis(3,4-dimethylbenzyl)amino)-1,2,4-triazin-5-yl)amino)ethyl)-5-cyclopropylpyrazolo[1,5-a]pyridin-7-yl)-3-azabicyclo[3.1.0]hexan-2-one. A solution of 3-(2-((R^{•})-1-((3-(bis(3,4-dimethylbenzyl)amino)-6-chloro-1,2,4-triazin-5-yl)amino)ethyl)-5-cyclopropylpyrazolo[1,5-a]pyridin-7-yl)-3-azabicyclo[3.1.0]hexan-2-one (270 mg, 0.41 mmol) and Pd/C (50 mg) in MeOH(6 mL) was stirred at room temperature for 2 h under H₂ atmosphere. The reaction mixture was filtered through a pad of Celite® and the filter cake was washed with DCM/MeOH (10/1, 50 mL). The filtrate was concentrated in vacuo. The residue was purified by preparative TLC, eluting with 5% MeOH in DCM to give 3-(2-((R^{•})-1-((3-(bis(3,4-dimethylbenzyl)amino)-1,2,4-triazin-5-yl)amino)ethyl)-5-cyclopropylpyrazolo[1,5-a]pyridin-7-yl)-3-azabicyclo[3.1.0]hexan-2-one (284 mg, crude) as a white solid. ESI-MS [M+H]⁺: 627.4.

Synthesis of 3-(2-((R^{•})-1-((3-amino-1,2,4-triazin-5-yl)amino)ethyl)-5-cyclopropylpyrazolo[1,5-a]pyridin-7-yl)-3-azabicyclo[3.1.0]hexan-2-one. A solution of 3-(2-((R^{•})-1-((3-(bis(3,4-dimethylbenzyl)amino)-1,2,4-triazin-5-yl)amino)ethyl)-5-cyclopropylpyrazolo[1,5-a]pyridin-7-yl)-3-azabicyclo[3.1.0]hexan-2-one (284 mg, crude) and TFA (2 mL, 0.04 mmol) in DCM (6 mL) was stirred at room temperature for 12 h. The reaction mixture was diluted with DCM/MeOH (V/V=10/1, 5 mL) and then adjust pH to 8-9 with NH₃ solution (7 M in MeOH). The mixture was concentrated in vacuo. The residue was purified by preparative TLC, eluting with 5% MeOH in DCM to give 3-(2-((R^{•})-1-((3-amino-1,2,4-triazin-5-yl)amino)ethyl)-5-cyclopropylpyrazolo[1,5-a]pyridin-7-yl)-3-azabicyclo[3.1.0]hexan-2-one (74 mg, 46% for 2 steps) as a white solid. ESI-MS [M+H]⁺: 391.2.

Synthesis of (S,2S)—N-(5-(((R^{•})-1-(5-cyclopropyl-7-(2-oxo-3-azabicyclo[3.1.0]hexan-3-yl)pyrazolo[1,5-a]pyridin-2-yl)ethyl)amino)-1,2,4-triazin-3-yl)-2-(4-methylpyrimidin-2-yl)cyclopropane-1-carboxamide. A solution of 3-(2-((R^{•})-1-((3-amino-1,2,4-triazin-5-yl)amino)ethyl)-5-cyclopropylpyrazolo[1,5-a]pyridin-7-yl)-3-azabicyclo[3.1.0]hexan-2-one (74 mg, 0.19 mmol), DIPEA (123 mg, 0.95 mmol), HATU (144 mg, 0.38 mmol) and (1S,2S)-2-(4-methylpyrimidin-2-yl)cyclopropane-1-carboxylic acid (50 mg, 0.28 mmol) in DCM (10 mL) was stirred at 50° C. for 8 h. The mixture was cooled to room temperature. The resulting mixture was diluted with H₂O (20 mL) and extracted with EtOAc (25 mL×3). The combined organic layers were washed with brine (20 mL), dried over Na₂SO₄, and concentrated in vacuo. The residue was purified by preparative TLC, eluting with 5% MeOH in DCM to give (1S,2S)—N-(5-(((1R^{•})-1-(5-cyclopropyl-7-(2-oxo-3-azabicyclo[3.1.0]hexan-3-yl)pyrazolo[1,5-a]pyridin-2-yl)ethyl)amino)-1,2,4-triazin-3-yl)-2-(4-methylpyrimidin-2-yl)cyclopropane-1-carboxamide (27 mg, 26%) as a white solid. ESI-MS [M+H]⁺: 551.3. ¹H NMR (400 MHz, DMSO) δ 10.61 (s, 1H), 8.58 (d, J=5.2 Hz, 1H), 8.50-8.41 (m, 1H), 8.31 (d, J=5.1 Hz, 1H), 7.29 (s, 1H), 7.13 (dd, J=9.7, 5.1 Hz, 1H), 6.58 (s, 1H), 6.42 (d, J=3.7 Hz, 1H), 5.35-5.30 (m, 1H), 4.12-4.07 (m, 1H), 3.70 (t, J=9.9 Hz, 1H), 2.56-2.52 (m, 2H), 2.43-2.33 (m, 3H), 2.19-2.08 (m, 1H), 2.02-1.93 (m, 2H), 1.57-1.51 (m, 5H), 1.22-1.18 (m, 1H), 1.08-0.88 (m, 3H), 0.76-0.72 (m, 2H).

### Example 106

### Synthesis of (1S,2S)—N-(6-(((R^{•})-1-(5-methyl-7-(3-methyl-2,4-dioxoimidazolidin-1-yl)pyrazolo[1,5-a]pyridin-2-yl)ethyl)amino)pyrimidin-4-yl)-2-(4-methylpyrimidin-2-yl)cyclopropane-1-carboxamide (I-106)

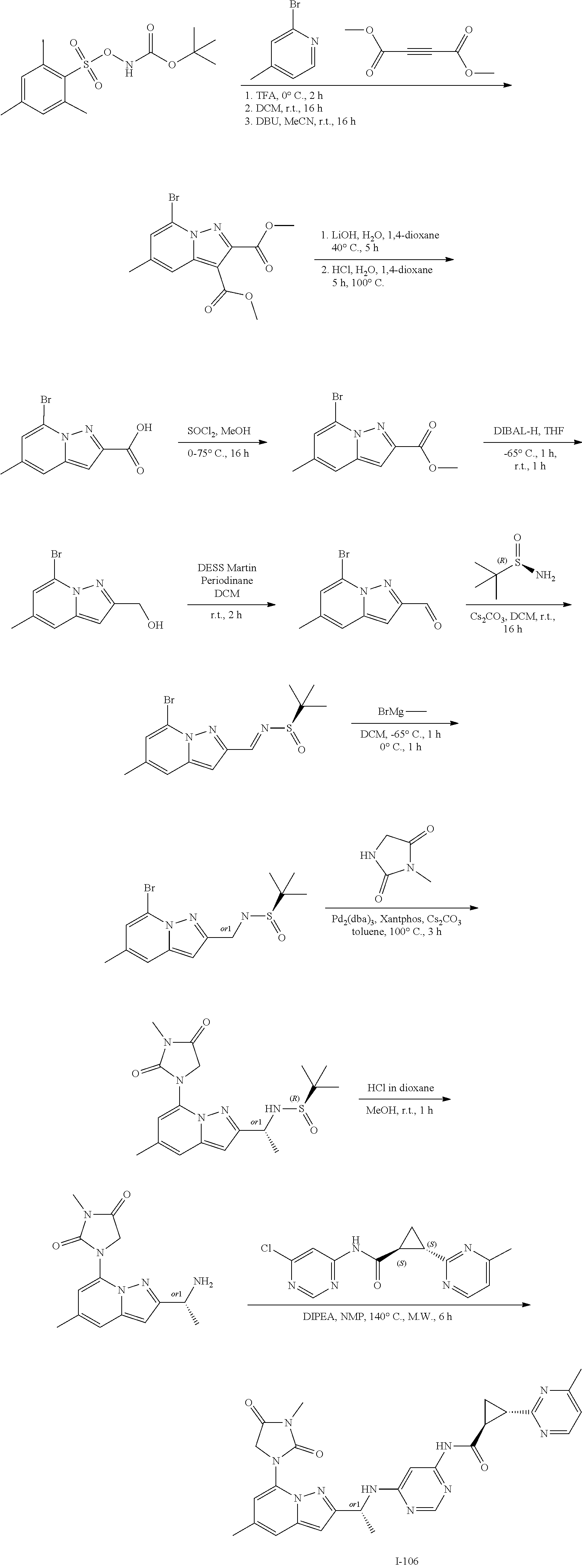

Synthesis of dimethyl 7-bromo-5-methylpyrazolo[1,5-a]pyridine-2,3-dicarboxylate. A solution of tert-butyl ((mesitylsulfonyl)oxy)carbamate (30 g, 95 mmol) in TFA (90 mL) was stirred at 0° C. for 2 h. The reaction mixture was added to the ice water (400 mL) dropwise and stirred for another 0.5 h. The mixture was filtered and the filter cake was washed with water (1 L). The filter cake was then collected and redissolved in DCM (250 mL), the resulting solution was dried over anhydrous Na₂SO₄, filtered to afford a solution of O-(mesitylsulfonyl)hydroxylamine in DCM (250 mL). 2-bromo-4-methylpyridine (7.5 g, 44 mmol) was added at 0° C. and the resulting mixture was stirred at room temperature for 16 h, then concentrated in vacuo. The residue was dissolved in MeCN (200 mL) and then dimethyl but-2-ynedioate (13 g, 88 mmol) and DBU (13 g, 88 mmol) were added dropwise at 0° C. After stirring at room temperature for another 16 h, the mixture was diluted in water (150 mL) and extracted with EtOAc (500 mL). The organics were dried over Na₂SO₄ and concentrated in vacuo. The residue was purified by column chromatography on silica gel, eluting with 5˜30% EtOAc in PE to give dimethyl 7-bromo-5-methylpyrazolo[1,5-a]pyridine-2,3-dicarboxylate (5.1 g, 36%) as a white solid. ESI-MS: [M+H]⁺, 326.9

Synthesis of 7-bromo-5-methylpyrazolo[1,5-a]pyridine-2-carboxylic acid. A solution of dimethyl 7-bromo-5-methylpyrazolo[1,5-a]pyridine-2,3-dicarboxylate (5.1 g, 16 mmol) and LiOH—H₂O (3.3 g, 78 mmol) in THF (75 mL) and H₂O (25 mL) was stirred at 40° C. for 5 h. The mixture was cooled to room temperature and concentrated in vacuo to remove THF. The residue was diluted in HCl (12 N in H₂O, 80 mL) and 1,4-dioxane (80 mL) and the resulting mixture was stirred at 100° C. for 10 h. The mixture was cooled to room temperature and concentrated in vacuo to give 7-bromo-5-methylpyrazolo[1,5-a]pyridine-2-carboxylic acid (4 g, quant). ESI-MS: [M+H]⁺, 255.0.

Synthesis of methyl 7-bromo-5-methylpyrazolo[1,5-a]pyridine-2-carboxylate. A solution of crude 7-bromo-5-methylpyrazolo[1,5-a]pyridine-2-carboxylic acid (4 g, 16 mmol) in MeOH (80 mL) was added SOCl₂ (9.4 g, 78 mmol) dropwise at 0° C. The resulting mixture was stirred at 75° C. for 5 h. The mixture was cooled to room temperature and then poured into ice water (100 mL) and then extracted with EtOAc (200 mL×2). The combined organic layers were washed with brine (100 mL), dried over Na₂SO₄, and concentrated in vacuo. The residue was purified by column chromatography on silica gel, eluting with 0-30% EtOAc in PE to give methyl 7-bromo-5-methyl pyrazolo[1,5-a]pyridine-2-carboxylate (3.3 g, yield 77%) as a white solid. ESI-MS: [M+H]⁺, 269.0.

Synthesis of (7-bromo-5-methylpyrazolo[1,5-a]pyridin-2-yl)methanol. To a solution of methyl 7-bromo-5-methylpyrazolo[1,5-a]pyridine-2-carboxylate (0.60 g, 2.2 mmol) in THF (12 mL) was added DIBAL-H (1N in hexanes, 6.6 mL, 6.6 mmol) at −65° C. After stirring at −65° C. for 1 h, the mixture was warmed to room temperature and stirred for another 1 h. The resulting mixture was quenched with NaOH (1 N in H₂O, 20 mL) and then extracted with DCM (90 mL×2). The combined organic layers were washed with brine (50 mL), dried over Na₂SO₄, and concentrated in vacuo. The residue was purified by column chromatography on silica gel, eluting with 0-30% EtOAc in PE to give (7-bromo-5-methylpyrazolo[1,5-a]pyridin-2-yl)methanol (0.50 g, 95%) as a white solid. ESI-MS: [M+H]⁺, 241.0.

Synthesis of 7-bromo-5-methylpyrazolo[1,5-a]pyridine-2-carbaldehyde. To a solution of (7-bromo-5-methylpyrazolo[1,5-a]pyridin-2-yl)methanol (0.50 g, 2.1 mol) in DCM (10 mL) was added Dess Martin periodinane (2.7 g, 6.3 mol) at 0° C. The mixture was stirred at room temperature for 2 h. The reaction mixture was quenched with Na₂S₂O₃ (sat. aq., 40 mL) and NaHCO₃ (sat. aq., 40 mL) then extracted with DCM (90 mL). The combined organics were dried over Na₂SO₄ and then concentrated in vacuo. The residue was purified by column chromatography on silica gel, eluting with 0-30% EtOAc in PE to give 7-bromo-5-methylpyrazolo[1,5-a]pyridine-2-carbaldehyde (0.46 g, 92%) as a yellow solid. ESI-MS: [M+H]⁺, 239.0.

Synthesis of (R,E)-N-((7-bromo-5-methylpyrazolo[1,5-a]pyridin-2-yl)methylene)-2-methylpropane-2-sulfinamide. A solution of 7-bromo-5-methylpyrazolo[1,5-a]pyridine-2-carbaldehyde (0.46 g, 1.9 mmol), (R)-2-methylpropane-2-sulfinamide (0.28 g, 2.3 mmol) and Cs₂CO₃ (1.2 g, 3.8 mmol) in DCM (10 mL) was stirred at room temperature for 16 h. The mixture was filtered and washed with DCM (80 mL). The filtrate was concentrated in vacuo. The residue was purified by column chromatography on silica gel, eluting with 60% EtOAc in PE to give (R,E)-N-((7-bromo-5-methylpyrazolo[1,5-a]pyridin-2-yl)methylene)-2-methylpropane-2-sulfinamide (0.46 g, 71%) as a white solid. ESI-MS: [M+H]⁺, 342.1.

Synthesis of (R)—N—((R^{•})-1-(7-bromo-5-methylpyrazolo[1,5-a]pyridin-2-yl)ethyl)-2-methylpropane-2-sulfinamide. To a solution of (R,E)-N-((7-bromo-5-methylpyrazolo[1,5-a]pyridin-2-yl)methylene)-2-methylpropane-2-sulfinamide (0.46 g, 1.35 mmol) in DCM (15 ml) was added CH₃MgBr (3 M in Et₂O, 0.90 mL, 2.7 mmol) at −65° C. The mixture was stirred at −65° C. for 1 h, then warm to −0° C. and stirred at this temperature for another 1.5 h under N₂ atmosphere. The reaction mixture was quenched with NH₄Cl (sat. aq., 20 mL) and extracted with DCM (30 mL×2). The combined organics were washed with brine (80 mL), dried over anhydrous Na₂SO₄, and then concentrated in vacuo. The residue was purified by column chromatography on silica gel, eluting with 30-50% EtOAc in PE to give (R)—N—((R^{•})-1-(7-bromo-5-methylpyrazolo[1,5-a]pyridin-2-yl)ethyl)-2-methylpropane-2-sulfinamide (0.21 g, 44%) as a white solid. ESI-MS: [M+H]⁺, 358.1.

Synthesis of (R)-2-methyl-N—((R^{•})-1-(5-methyl-7-(3-methyl-2,4-dioxoimidazolidin-1-yl)pyrazolo[1,5-a]pyridin-2-yl)ethyl)propane-2-sulfinamide. A solution of (R)—N—((R^{•})-1-(7-bromo-5-methylpyrazolo[1,5-a]pyridin-2-yl)ethyl)-2-methylpropane-2-sulfinamide (0.21 g, 0.59 mmol), 3-methylimidazolidine-2,4-dione (0.20 g, 1.8 mmol), Pd₂(dba)₃ (0.11 g, 0.12 mmol), Xantphos (0.14 g, 0.24 mmol) and Cs₂CO₃ (0.59 g, 1.8 mmol) in toluene (13 mL) was stirred at 100° C. for 3 h under a N₂ atmosphere. The mixture was cooled to room temperature. The reaction mixture was filtered through a pad of Celite® and the filter cake was washed with DCM (90 mL). The filtrate was concentrated in vacuo and the residue was purified by column chromatography on silica gel, eluting with 20-60% EtOAc in PE to give (R)-2-methyl-N—((R^{•})-1-(5-methyl-7-(3-methyl-2,4-dioxoimidazolidin-1-yl)pyrazolo[1,5-a]pyridin-2-yl)ethyl)propane-2-sulfinamide (0.11 g, 48%) as a yellow solid. ESI-MS: [M+H]⁺, 392.3.

Synthesis of (R^{•})-1-(2-(1-aminoethyl)-5-methylpyrazolo[1,5-a]pyridin-7-yl)-3-methylimidazolidine-2,4-dione. To a solution of (R)-2-methyl-N—((R^{•})-1-(5-methyl-7-(3-methyl-2,4-dioxoimidazolidin-1-yl)pyrazolo[1,5-a]pyridin-2-yl)ethyl)propane-2-sulfinamide (0.11 g, 0.28 mmol) in MeOH (2 mL) was added HCl (4 N solution in dioxane, 2 mL, 8 mmol) at 0° C. The resulting mixture was stirred at room temperature for 30 min. The reaction mixture was concentrated in vacuo and NH₃ (7 N in MeOH 5.0 mL) was added. The mixture was stirred for a further 10 min and then concentrated in vacuo. The residue was purified by preparative TLC, eluting with 5% MeOH in DCM to give (R^{•})-1-(2-(1-aminoethyl)-5-methylpyrazolo[1,5-a]pyridin-7-yl)-3-methylimidazolidine-2,4-dione (70 mg, 87%) as a yellow solid. ESI-MS: [M+H]⁺, 288.2

Synthesis of (S,2S)—N-(6-(((R^{•})-1-(5-methyl-7-(3-methyl-2,4-dioxoimidazolidin-1-yl)pyrazolo[1,5-a]pyridin-2-yl)ethyl)amino)pyrimidin-4-yl)-2-(4-methylpyrimidin-2-yl)cyclopropane-1-carboxamide. A solution of (R^{•})-1-(2-(1-aminoethyl)-5-methylpyrazolo[1,5-a]pyridin-7-yl)-3-methylimidazolidine-2,4-dione (70 mg, 0.24 mmol), (1S,2S)—N-(6-chloropyrimidin-4-yl)-2-(4-methylpyrimidin-2-yl)cyclopropane-1-carboxamide (70 mg, 0.24 mmol) and DIPEA (0.15 g, 1.2 mmol) in NMP (5 mL) was stirred in a sealed tube. After degassing with N₂ for 1 min, the reaction was irradiated in a microwave reactor at 140° C. for 6 h. The reaction was quenched with water (60 mL) and then extracted with EtOAc (90 mL×2). The combined organics were washed with water (50 mL×3) and brine (50 mL), dried over Na₂SO₄, and concentrated in vacuo. The residue was purified by column chromatography on silica gel, eluting with 0-5% MeOH in DCM to give (1S,2S)—N-(6-(((R^{•})-1-(5-methyl-7-(3-methyl-2,4-dioxoimidazolidin-1-yl)pyrazolo[1,5-a]pyridin-2-yl)ethyl)amino)pyrimidin-4-yl)-2-(4-methylpyrimidin-2-yl)cyclopropane-1-carboxamide (44 mg, 34%) as a white solid. ESI-MS: [M+H]⁺, 541.2, ¹H NMR (400 MHz, DMSO) δ 10.65 (s, 1H), 8.52 (d, J=5.0 Hz, 1H), 8.15 (s, 1H), 7.83 (s, 1H), 7.41 (s, 1H), 7.30 (s, 1H), 7.21 (d, J=5.0 Hz, 1H), 6.85 (s, 1H), 6.45 (s, 1H), 5.46 (s, 1H), 4.68 (s, 2H), 3.00 (s, 3H), 2.61-2.59 (m, 1H), 2.58-2.54 (m, 1H), 2.41 (s, 3H), 2.35 (s, 3H), 1.60-1.43 (m, 5H).

### Example 107

### Synthesis of (2S,2S)—N-(6-(((R^{•})-1-(6-cyclopropyl-8-(3-methyl-2,4-dioxoimidazolidin-1-yl)-[1,2,4]triazolo[1,5-a]pyridin-2-yl)ethyl)amino)pyrimidin-4-yl)-2-(4-methylpyrimidin-2-yl)cyclopropane-1-carboxamide, isomer 1 (I-107)

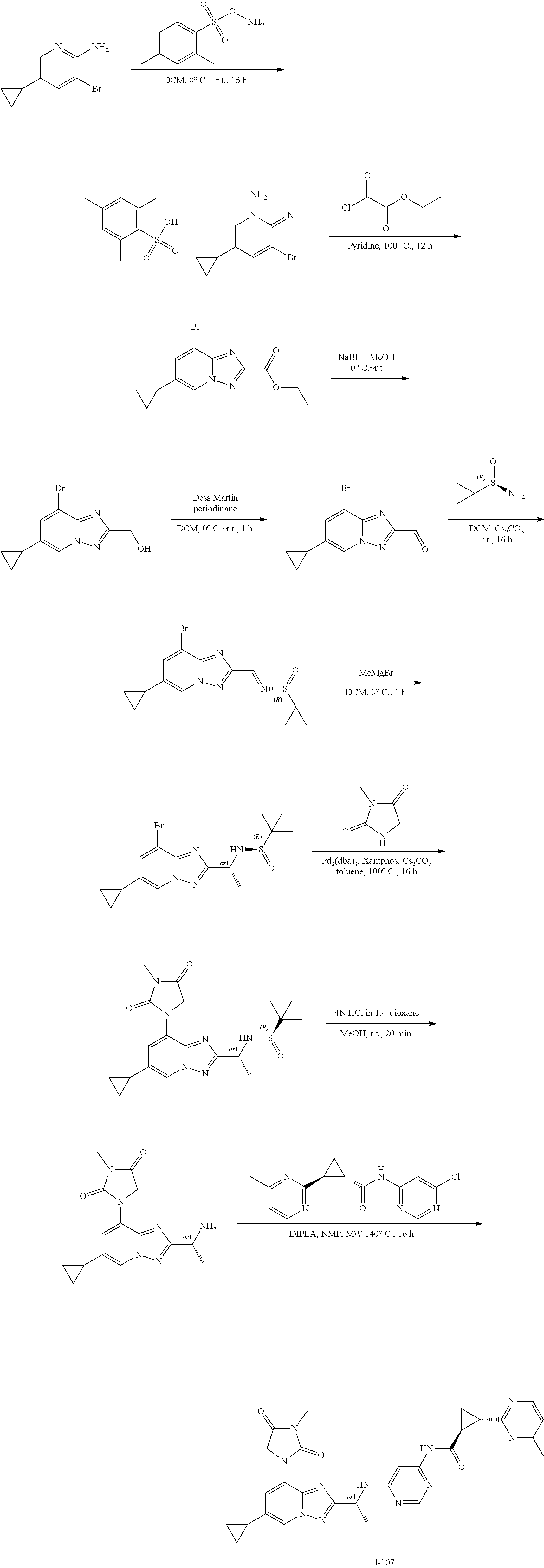

Synthesis of 3-bromo-5-cyclopropyl-2-iminopyridin-1 (2H)-amine 2,4,6-trimethylbenzenesulfonate. To a stirred solution of O-(mesitylsulfonyl)hydroxylamine (20 g, 93 mmol) in DCM (100 mL) was added 3-bromo-5-cyclopropylpyridin-2-amine (4.0 g, 19 mmol) slowly at 0° C. The mixture was stirred at 0° C. for 10 min and at room temperature for 16 h. The reaction mixture was filtered and the filtrant was dried in vacuo to afford 3-bromo-5-cyclopropyl-2-iminopyridin-1 (2H)-amine 2,4,6-trimethylbenzenesulfonate (8.1 g, quant.) as a yellow solid. ESI-MS [M+H]⁺: 228.0.

Synthesis of ethyl 8-bromo-6-cyclopropyl-[1,2,4]triazolo[1,5-a]pyridine-2-carboxylate. To a stirred solution of 3-bromo-5-cyclopropyl-2-iminopyridin-1 (2H)-amine 2,4,6-trimethylbenzenesulfonate (8.1 g, 19 mmol) in pyridine (80 mL) was added dropwise ethyl 2-chloro-2-oxoacetate (10 g, 73 mmol) at room temperature. The mixture was stirred at 100° C. for 12 h. After cooling to room temperature, the reaction mixture was concentrated and diluted with EtOAc (150 mL). The organic layer was washed with water (150 mL×2) and brine (150 mL), dried over Na₂SO₄ and concentrated to give the crude product, which was purified by column chromatography (eluent: EtOAc/PE=1/4) to give ethyl 8-bromo-6-cyclopropyl-[1,2,4]triazolo[1,5-a]pyridine-2-carboxylate (2.0 g, 34%) as a yellow solid. ESI-MS [M+H]⁺: 310.0.

Synthesis of (8-bromo-6-cyclopropyl-[1,2,4]triazolo[1,5-a]pyridin-2-yl)methanol. To a stirred solution of ethyl 8-bromo-6-cyclopropyl-[1,2,4]triazolo[1,5-a]pyridine-2-carboxylate (1.0 g, 3.2 mmol) in MeOH (20 mL) was added NaBH₄ (365 mg, 9.6 mmol) in portions at 0° C. The mixture was stirred at room temperature for 3 h. The reaction mixture was quenched with NH₄Cl (sat. aq., 50 mL), extracted with DCM (30 ml×3). The combined organics were washed with brine (30 mL), dried over Na₂SO₄, concentrated to give the crude product, which was purified by column chromatography (eluent: EtOAc/PE=1/2) to give (8-bromo-6-cyclopropyl-[1,2,4]triazolo[1,5-a]pyridin-2-yl)methanol (580 mg, 68%) as a yellow solid. ESI-MS [M+H]⁺: 268.1.

Synthesis of 8-bromo-6-cyclopropyl-[1,2,4]triazolo[1,5-a]pyridine-2-carbaldehyde. To a stirred solution of (8-bromo-6-cyclopropyl-[1,2,4]triazolo[1,5-a]pyridin-2-yl)methanol (580 mg, 2.2 mmol) in DCM (15 mL) was added Dess Martin periodinane (1.8 g, 4.2 mmol) at 0° C. The mixture was stirred at room temperature for 1 h then was diluted with DCM (100 mL) and quenched with Na₂S₂O₃ aqueous (30 mL, 10%). The organic layer was washed with NaHCO₃ (sat. aq., 50 mL) and brine (50 mL), dried over anhydrous Na₂SO₄, concentrated in vacuo to give 8-bromo-6-cyclopropyl-[1,2,4]triazolo[1,5-a]pyridine-2-carbaldehyde (580 mg, quant.) as a white solid, which was used in next step without further purification. ESI-MS [M+H]⁺: 266.0.

Synthesis of (R,E)-N-((8-bromo-6-cyclopropyl-[1,2,4]triazolo[1,5-a]pyridin-2-yl)methylene)-2-methylpropane-2-sulfinamide. To a stirred solution of 8-bromo-6-cyclopropyl-[1,2,4]triazolo[1,5-a]pyridine-2-carbaldehyde (0.58 g, 2.2 mmol) and (R)-2-methylpropane-2-sulfinamide (0.32 g, 2.6 mmol) in DCM (15 mL) was added Cs₂CO₃ (1.4 g, 4.4 mmol) at room temperature. The mixture was stirred at room temperature for 16 h. The reaction mixture was filtered and washed with DCM (20 mL). The filtrate was concentrated in vacuo to give the crude, which was purified by column chromatography (eluent: EtOAc/PE=1/5) to give (R,E)-N-((8-bromo-6-cyclopropyl-[1,2,4]triazolo[1,5-a]pyridin-2-yl)methylene)-2-methylpropane-2-sulfinamide (720 mg, 89%) as a yellow solid. ESI-MS [M+H]⁺: 369.1.

Synthesis of (R)—N—((R^{•})-1-(8-bromo-6-cyclopropyl-[1,2,4]triazolo[1,5-a]pyridin-2-yl)ethyl)-2-methylpropane-2-sulfinamide, isomer 1 and (R)—N—((R^{•})-1-(8-bromo-6-cyclopropyl-[1,2,4]triazolo[1,5-a]pyridin-2-yl)ethyl)-2-methylpropane-2-sulfinamide, isomer 2. To a stirred solution of (R,E)-N-((8-bromo-6-cyclopropyl-[1,2,4]triazolo[1,5-a]pyridin-2-yl)methylene)-2-methylpropane-2-sulfinamide (520 mg, 1.4 mmol) in DCM (15 mL) was added dropwise CH₃MgBr (0.95 mL, 2.8 mmol, 3M in ether) at 0° C. and the mixture was stirred at 0° C. for 1 h. The reaction mixture was quenched with NH₄Cl (sat. aq., 100 mL) and extracted with DCM (30 mL×2). The combined organics were washed with brine (30 mL), dried over anhydrous Na₂SO₄ and concentrated to give the crude product, which was purified by column chromatography (eluent: EtOAc/PE=1/2˜1/1) to give (R)—N—((R^{•})-1-(8-bromo-6-cyclopropyl-[1,2,4]triazolo[1,5-a]pyridin-2-yl)ethyl)-2-methylpropane-2-sulfinamide, isomer 1 (400 mg, 74%) and (R)—N—((R^{•})-1-(8-bromo-6-cyclopropyl-[1,2,4]triazolo[1,5-a]pyridin-2-yl)ethyl)-2-methylpropane-2-sulfinamide, isomer 2 (100 mg, 18%) as a yellow solid. ESI-MS [M+H]⁺: 385.1.

Synthesis of (R)—N—((R^{•})-1-(6-cyclopropyl-8-(3-methyl-2,4-dioxoimidazolidin-1-yl)-[1,2,4]triazolo[1,5-a]pyridin-2-yl)ethyl)-2-methylpropane-2-sulfinamide. A mixture of (R)—N—((R^{•})-1-(8-bromo-6-cyclopropyl-[1,2,4]triazolo[1,5-a]pyridin-2-yl)ethyl)-2-methylpropane-2-sulfinamide, isomer 1 (380 mg, 0.99 mmol), 3-methylimidazolidine-2,4-dione (340 mg, 3.0 mmol), Pd₂(dba)₃ (92 mg, 0.1 mmol), Xantphos (116 mg, 0.2 mmol) and Cs₂CO₃ (978 mg, 3.0 mmol) in toluene (10 mL) was stirred at 100° C. for 16 h under N₂. After cooling to room temperature, the reaction mixture was filtered through Celite® and the filter cake was washed with DCM/MeOH (10/1, 50 mL). The filtrate was concentrated in vacuo to give the crude product, which was purified by column chromatography (eluent: EtOAc/PE=1/5˜1/1) to give (R)—N—((R^{•})-1-(6-cyclopropyl-8-(3-methyl-2,4-dioxoimidazolidin-1-yl)-[1,2,4]triazolo[1,5-a]pyridin-2-yl)ethyl)-2-methylpropane-2-sulfinamide, isomer 1 (270 mg, 65%) as a yellow solid. ESI-MS [M+H]⁺: 419.2.

Synthesis of (R^{•})-1-(2-(1-aminoethyl)-6-cyclopropyl-[1,2,4]triazolo[1,5-a]pyridin-8-yl)-3-methylimidazolidine-2,4-dione, isomer 1. To a solution of (R)—N—((R^{•})-1-(6-cyclopropyl-8-(3-methyl-2,4-dioxoimidazolidin-1-yl)-[1,2,4]triazolo[1,5-a]pyridin-2-yl)ethyl)-2-methylpropane-2-sulfinamide, isomer 1 (270 mg, 0.65 mmol) in MeOH (3.0 mL) was added HCl (4 M in 1,4-dioxane, 3.0 mL). The resulting mixture was stirred at room temperature for 30 min. The reaction mixture was concentrated and NH₃ (7 M in MeOH, 5.0 mL) was added. The mixture was stirred for 10 min and concentrated in vacuo to give the crude, which was purified by preparative TLC (eluent: DCM/NH₃ (7 M in MeOH)=10/1) to give (R^{•})-1-(2-(1-aminoethyl)-6-cyclopropyl-[1,2,4]triazolo[1,5-a]pyridin-8-yl)-3-methylimidazolidine-2,4-dione, isomer 1 (150 mg, 74%) as a white solid. ESI-MS [M+H]⁺: 315.2.

Synthesis of (1S,2S)—N-(6-(((R^{•})-1-(6-cyclopropyl-8-(3-methyl-2,4-dioxoimidazolidin-1-yl)-[1,2,4]triazolo[1,5-a]pyridin-2-yl)ethyl)amino)pyrimidin-4-yl)-2-(4-methylpyrimidin-2-yl)cyclopropane-1-carboxamide, isomer 1. A mixture of (R^{•})-1-(2-(1-aminoethyl)-6-cyclopropyl-[1,2,4]triazolo[1,5-a]pyridin-8-yl)-3-methylimidazolidine-2,4-dione, isomer 1 (65 mg, 0.21 mmol), (1S,2S)—N-(6-chloropyrimidin-4-yl)-2-(4-methylpyrimidin-2-yl)cyclopropane-1-carboxamide (61 mg, 0.21 mmol) and DIPEA (81 mg, 0.63 mmol) in NMP (3.0 mL) was stirred in a sealed tube. After degassing with N₂ for 1 min, the reaction was irradiated in a microwave reactor at 140° C. for 16 h. After cooling to room temperature, the reaction mixture was poured into water (20 mL) and extracted with EtOAc (20 ml×3). The combined organics were washed with brine (20 mL), dried over Na₂SO₄, concentrated to give the crude product, which was purified by column chromatography (eluent: DCM/MeOH=20/1) then preparative TLC (eluent: DCM/MeOH=15/1) to give (1S,2S)—N-(6-(((R^{•})-1-(6-cyclopropyl-8-(3-methyl-2,4-dioxoimidazolidin-1-yl)-[1,2,4]triazolo[1,5-a]pyridin-2-yl)ethyl)amino)pyrimidin-4-yl)-2-(4-methylpyrimidin-2-yl)cyclopropane-1-carboxamide, isomer 1 (48 mg, 40%) as a white solid. ESI-MS [M+H]⁺: 568.3. ¹H NMR (400 MHz, DMSO) δ 10.66 (s, 1H), 8.60 (d, J=1.4 Hz, 1H), 8.52 (d, J=5.1 Hz, 1H), 8.14 (s, 1H), 7.95 (s, 1H), 7.79 (d, J=1.4 Hz, 1H), 7.32 (s, 1H), 7.21 (d, J=5.1 Hz, 1H), 5.47 (s, 1H), 4.90 (s, 2H), 2.97 (s, 3H), 2.65-2.59 (m, 1H), 2.57-2.53 (m, 1H), 2.41 (s, 3H), 2.08-1.99 (m, 1H), 1.61-1.47 (m, 5H), 1.01-0.97 (m, 2H), 0.76-0.72 (m, 2H).

### Example 108

### Synthesis of (2S,2S)—N-(6-(((R^{•})-1-(6-cyclopropyl-8-(3-methyl-2,4-dioxoimidazolidin-1-yl)-[1,2,4]triazolo[1,5-a]pyridin-2-yl)ethyl)amino)pyrimidin-4-yl)-2-(4-methylpyrimidin-2-yl) cyclopropane-1-carboxamide, isomer 2 (I-108)

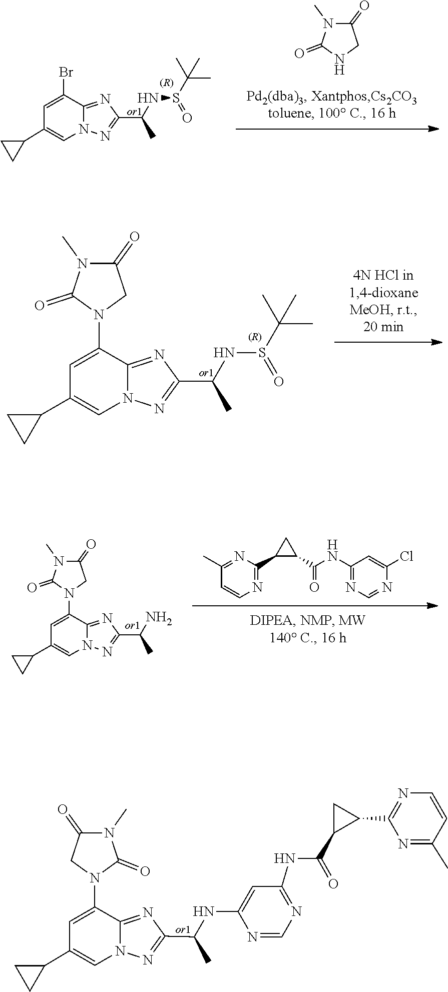

Synthesis of (R)—N—((R^{•})-1-(6-cyclopropyl-8-(3-methyl-2,4-dioxoimidazolidin-1-yl)-[1,2,4]triazolo[1,5-a]pyridin-2-yl)ethyl)-2-methylpropane-2-sulfinamide, isomer 2. The mixture of (R)—N—((R^{•})-1-(8-bromo-6-cyclopropyl-[1,2,4]triazolo[1,5-a]pyridin-2-yl)ethyl)-2-methylpropane-2-sulfinamide, isomer 2 (110 mg, 0.29 mmol), 3-methylimidazolidine-2,4-dione (99 mg, 0.87 mmol), Pd₂(dba)₃ (27 mg, 0.029 mmol), Xantphos (34 mg, 0.058 mmol) and Cs₂CO₃ (284 mg, 0.87 mmol) in toluene (6 mL) was stirred at 100° C. for 16 h under N₂. After cooling to room temperature, the reaction mixture was filtered through Celite® and the filter cake was washed with DCM/MeOH (10/1, 50 mL). The filtrate was concentrated in vacuo to give the crude product, which was purified by silica gel chromatography (eluent: EtOAc/PE=1/5˜1/1) to give (R)—N—((R^{•})-1-(6-cyclopropyl-8-(3-methyl-2,4-dioxoimidazolidin-1-yl)-[1,2,4]triazolo[1,5-a]pyridin-2-yl)ethyl)-2-methylpropane-2-sulfinamide, isomer 2 (80 mg, 66%) as a yellow solid. ESI-MS [M+H]⁺: 419.2.

Synthesis of (R^{•})-1-(2-(1-aminoethyl)-6-cyclopropyl-[1,2,4]triazolo[1,5-a]pyridin-8-yl)-3-methylimidazolidine-2,4-dione, isomer 2. To a solution of (R)—N—((R^{•})-1-(6-cyclopropyl-8-(3-methyl-2,4-dioxoimidazolidin-1-yl)-[1,2,4]triazolo[1,5-a]pyridin-2-yl)ethyl)-2-methylpropane-2-sulfinamide, isomer 2 (80 mg, 0.19 mmol) in MeOH (2.0 mL) was added HCl (4 M in 1,4-dioxane, 2.0 mL). The resulting mixture was stirred at room temperature for 30 min. The reaction mixture was concentrated in vacuo and NH₃ (7 M in MeOH, 5.0 mL) was added. The mixture was stirred for 10 min and concentrated in vacuo to give the crude, which was purified by preparative TLC (eluent: DCM/NH₃ (7 M in MeOH)=10/1) to give (R^{•})-1-(2-(1-aminoethyl)-6-cyclopropyl-[1,2,4]triazolo [1,5-a]pyridin-8-yl)-3-methylimidazolidine-2,4-dione, isomer 2 (45 mg, 75%) as a white solid. ESI-MS [M+H]⁺: 315.2.

Synthesis of (2S,2S)—N-(6-(((R^{•})-1-(6-cyclopropyl-8-(3-methyl-2,4-dioxoimidazolidin-1-yl)-[1,2,4]triazolo[1,5-a]pyridin-2-yl)ethyl)amino)pyrimidin-4-yl)-2-(4-methylpyrimidin-2-yl)cyclopropane-1-carboxamide, isomer 2. A mixture of (R^{•})-1-(2-(1-aminoethyl)-6-cyclopropyl-[1,2,4]triazolo[1,5-a]pyridin-8-yl)-3-methylimidazolidine-2,4-dione, isomer 2 (45 mg, 0.14 mmol), (1S,2S)—N-(6-chloropyrimidin-4-yl)-2-(4-methylpyrimidin-2-yl)cyclopropane-1-carboxamide (40 mg, 0.14 mmol) and DIPEA (54 mg, 0.42 mmol) in NMP (2.0 mL) was stirred in a sealed tube. After degassing with N₂ for 1 min, the reaction was irradiated in a microwave reactor at 140° C. for 16 h. After cooling to room temperature, the reaction mixture was poured into water (20 mL) and extracted with EtOAc (20 mL×3). The combined organics were washed with water (50 mL×3) and brine (50 mL), dried over Na₂SO₄ then concentrated in vacuo to give the crude product, which was purified by silica gel chromatography (eluent: DCM/MeOH=20/1) and Prep—TLC (eluent: DCM/MeOH=15/1) to give (1S,2S)—N-(6-(((R^{•})-1-(6-cyclopropyl-8-(3-methyl-2,4-dioxoimidazolidin-1-yl)-[1,2,4]triazolo[1,5-a]pyridin-2-yl)ethyl)amino)pyrimidin-4-yl)-2-(4-methylpyrimidin-2-yl)cyclopropane-1-carboxamide, isomer 2 (30 mg, 38%) as a white solid. ESI-MS [M+H]⁺: 568.3. ¹H NMR (400 MHz, DMSO) δ 10.66 (s, 1H), 8.60 (d, J=1.3 Hz, 1H), 8.52 (d, J=5.1 Hz, 1H), 8.14 (s, 1H), 7.94-7.89 (m, 1H), 7.79 (d, J=1.4 Hz, 1H), 7.32 (s, 1H), 7.21 (d, J=5.1 Hz, 1H), 5.47 (s, 1H), 4.90 (s, 2H), 2.97 (s, 3H), 2.64-2.59 (m, 1H), 2.57-2.52 (m, 1H), 2.41 (s, 3H), 2.08-2.01 (m, 1H), 1.60-1.47 (m, 5H), 1.02-0.96 (m, 2H), 0.77-0.72 (m, 2H).

### Example 109

### Inhibitory Activity of Exemplary Compounds Against Plasma Kallikrein.

Compounds were evaluated for inhibition of the human activated kallikrein enzyme in two formats of an assay employing a fluorogenic peptide substrate. In one assay format, the concentrations of reagents were as follows: 20 mM Tris pH 7.5, 1 mM EDTA, 150 mM sodium chloride, 0.1% PEG-400, 0.1% Triton X-100, 500 pM activated kallikrein enzyme, 300 pM Pro-Phe-Arg-7-amido-4-methylcoumarin (PFR-AMC) substrate. Prior to reaction initiation with substrate, enzyme and inhibitors were preincubated for 30 min at room temperature. After initiation with substrate, reactions were incubated for 10 min at room temperature and fluorescence emission at 460 nm from 380 nm excitation measured with a microplate reader. In another assay format, the concentrations of reagents were as follows: 20 mM Tris pH 7.5, 1 mM EDTA, 150 mM sodium chloride, 0.1% PEG-400, 0.1% Triton X-100, 5 pM activated kallikrein enzyme, 300 uM PFR-AMC substrate. Prior to reaction initiation with substrate, enzyme and inhibitors were preincubated for 30 min at room temperature. After initiation with substrate, reactions were incubated for 18 hr at room temperature and fluorescence emission at 460 nm from 380 nm excitation measured with a microplate reader.

Table 8 provides the results of the assay in the format with 500 pM activated kallikrein assay. For the compounds listed in Table 1, the EC₅₀ values are reported according to the following ranges: A≤2.5 nM; 2.5 nM<B≤25 nM; 25 nM<C≤500 nM; 500 nM<D.

TABLE 8

| | Primary Dose |
|---|---|
| | Response Assay: |
| Compound | Average EC50 |
| 1 | D |
| 2 | D |
| 3 | B |
| 4 | D |
| 5 | D |
| 6 | D |
| 7 | D |
| 8 | C |
| 9 | D |
| 10 | D |
| 11 | D |
| 12 | D |
| 13 | D |
| 14 | A |
| 15 | C |
| 16 | A |
| 17 | B |
| 18 | B |
| 19 | D |
| 20 | D |
| 21 | D |
| 22 | A |
| 23 | A |
| 24 | B |
| 25 | A |
| 26 | C |
| 27 | B |
| 28 | D |
| 29 | B |
| 30 | A |
| 31 | A |
| 32 | B |
| 33 | B |
| 34 | B |
| 35 | A |
| 36 | B |
| 37 | D |
| 38 | C |
| 39 | A |
| 40 | A |
| 41 | A |
| 42 | A |
| 43 | B |
| 44 | B |
| 45 | A |
| 46 | A |
| 47 | B |
| 48 | B |
| 49 | A |
| 50 | D |
| 51 | B |
| 52 | B |
| 53 | A |
| 54 | A |
| 55 | B |
| 56 | A |
| 57 | A |
| 58 | B |
| 59 | B |
| 60 | B |
| 61 | A |
| 62 | B |
| 63 | A |
| 64 | D |
| 65 | D |
| 66 | A |
| 67 | C |
| 68 | A |
| 69 | B |
| 70 | D |
| 71 | B |
| 72 | A |
| 73 | A |
| 74 | D |
| 75 | B |
| 76 | C |
| 77 | A |
| 78 | B |
| 79 | B |
| 80 | A |
| 81 | A |
| 82 | C |
| 83 | A |
| 84 | A |
| 85 | A |
| 86 | A |
| 87 | A |
| 88 | B |
| 89 | A |
| 90 | A |
| 91 | B |
| 92 | B |
| 93 | A |
| 94 | A |
| 95 | A |
| 96 | C |
| 97 | A |
| 98 | A |
| 99 | A |
| 100 | A |
| 101 | A |
| 102 | A |
| 103 | A |
| 104 | B |
| 105 | A |
| 106 | A |
| 107 | A |
| 108 | B |

### Example 110

### Neat Human and Rat Plasma Assay (Fluorogenic Peptide).

To analyze inhibition of plasma kallikrein in an ex vivo setting, the potency of compounds was measure in contact pathway-activated plasma assays. In a fluorogenic peptide substrate assay, test compounds dissolved in DMSO were added to sodium citrate collected human or rat plasma in a 96-well microplate. Alternatively, citrated plasma was collected from rats administered the compounds orally or by IV. 10 nM of human FXIIa (Enzyme Research Laboratories) diluted in PKa buffer (20 mM Tris-HCl, pH 7.5, 150 mM NaCl, 1 mM EDTA, 0.100 PEG-8000, and 0.1% Triton X-100) was added to the plasma, followed by the 100 μM of the profluorescent, synthetic plasma kallikrein substrate PFR-AMC (also diluted in PKa buffer). Final plasma concentration in the reaction was 780. Fluorescence was immediately monitored by excitation/emission wavelengths of 360 nm/480 nm respectively over a period of 5 minutes in a microplate reader. The resulting linear increase in fluorescence emission (reflecting PKa proteolysis of PFR-AMC substrate) was fit to extract a proteolytic rate (fluorescent units over time), and this rate was subsequently plotted against compound inhibitor concentration. Resulting plots were fit to a standard 4-parameter IC50/C90 equation to determine min/max values, IC50/90, and slope. All experimental steps were performed at room temperature. Table 2 provides results of the assay.

For the compounds listed in Table 9, the IC₉₀ values are reported according to the following ranges: A≤500 nM; 500 nM<B≤5000 nM; 5000 nM<C≤65000 nM.

TABLE 9

| Compound | Human Plasma |
|---|---|
| “I-” | Assay: IC90 |
| 22 | C |
| 23 | C |
| 25 | C |
| 29 | C |
| 31 | B |
| 35 | B |
| 39 | C |
| 40 | B |
| 41 | C |
| 42 | B |
| 45 | C |
| 46 | C |
| 49 | B |
| 52 | B |
| 53 | B |
| 54 | B |
| 56 | C |
| 57 | B |
| 60 | B |
| 61 | B |
| 63 | B |
| 66 | B |
| 71 | B |
| 72 | B |
| 73 | A |
| 75 | B |
| 77 | B |
| 80 | A |
| 81 | A |
| 83 | A |
| 84 | A |
| 85 | A |
| 86 | B |
| 87 | B |
| 88 | C |
| 89 | B |
| 90 | C |
| 93 | A |
| 94 | A |
| 95 | A |
| 97 | A |
| 98 | A |
| 99 | A |
| 100 | A |
| 101 | A |
| 102 | A |
| 103 | A |
| 105 | A |
| 106 | A |
| 107 | A |

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of examples.

### EXEMPLARY ENUMERATED EMBODIMENTS

1. A compound of Formula (I):

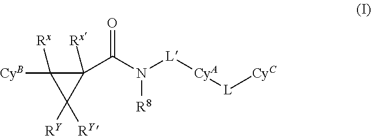

or a pharmaceutically acceptable salt thereof,
wherein:
Cy^{A} is a phenylene, a 5- to 6-membered monocyclic heteroarylene having 1-3 heteroatoms independently selected from oxygen, nitrogen, and sulfur, or a 7- to 12-membered bicyclic heteroarylene having 1-4 heteroatoms independently selected from oxygen, nitrogen, and sulfur, wherein Cy^{A} is substituted with 0-4 -R^{A} groups;
   each R^{A} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, phenyl, 3- to 7-membered saturated or partially unsaturated monocyclic carbocyclyl, or 3- to 7-membered saturated or partially unsaturated monocyclic heterocyclyl having 1-2 heteroatoms selected from oxygen, nitrogen, or sulfur;
each R is independently hydrogen or an optionally substituted C₁₋₆ aliphatic group;
each R^{Y} and R^{Y′} is independently selected from hydrogen, halogen, and an optionally substituted C₁₋₆ aliphatic group;
each R^{x} and R^{x′} is independently selected from hydrogen, halogen, or —CN;
Cy^{B} is selected from phenyl, 8- to 10-membered bicyclic aryl, a 5- to 6-membered heteroaryl having 1-3 heteroatoms independently selected from oxygen, nitrogen, and sulfur or a 7- to 10-membered heteroaryl having 1-4 heteroatoms independently selected from oxygen, nitrogen, and sulfur, wherein Cy^{B} is substituted with 0-4 -R^{B} groups; or
Cy^{B} and R^{x}, together with their intervening atoms, form a 6- to 12-membered spirocyclic ring system having 0-4 heteroatoms independently selected from oxygen, nitrogen, and sulfur, wherein the ring or rings formed by Cy^{B} and R^{x} may be substituted with 0-4 -R^{B} groups;
   each R^{B} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, a 3- to 7-membered saturated or partially unsaturated monocyclic heterocyclyl having 1-2 heteroatoms selected from oxygen, nitrogen, or sulfur, or a 5- to 6-membered heteroaryl having 1-4 heteroatoms independently selected from oxygen, nitrogen, and sulfur;
L is an optionally substituted C₁₋₃ hydrocarbon chain, wherein 1 to 3 methylene units are optionally and independently replaced with —C(O)—, —O—, —NR^{z}—, —S—, —SO—, —SO₂—, an optionally substituted cyclopropylene, or an optionally substituted 5- to 6-membered saturated or partially unsaturated heterocyclene, having 1-3 heteroatoms independently selected from oxygen, nitrogen, and sulfur;
   each R^{z} is independently selected from hydrogen, —(CH₂)₀₋₃OR, —(CH₂)₀₋₃C(O)OR, or an optionally substituted C₁₋₆ aliphatic group;
L′ is a covalent bond or an optionally substituted C₁₋₃ hydrocarbon chain, wherein 1 to 3 methylene units are optionally and independently replaced with —O—, —NR^{z}—, —S—, —SO—, or SO₂—;
Cy^{C} is selected from a 5- to 6-membered heteroaryl having 1-3 heteroatoms independently selected from oxygen, nitrogen, and sulfur, phenyl, 8- to 10-membered bicyclic aryl, a 7- to 10-membered heteroaryl having 1-4 heteroatoms independently selected from oxygen, nitrogen, and sulfur, or a 6- to 12-membered saturated or partially unsaturated fused bicyclic heterocyclyl having 1-4 heteroatoms independently selected from oxygen, nitrogen, or sulfur, wherein Cy^{C} is substituted with 0-6 -L^{C}-R^{C} groups;
   each L^{C} is independently selected from a covalent bond or an optionally substituted C₁₋₆ hydrocarbon chain, wherein 1 to 3 methylene units are optionally and independently replaced with —O— or —NR—;
   each R^{C} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, phenyl, a 3- to 7-membered saturated or partially unsaturated monocyclic carbocyclyl, a 5- or 6-membered heteroaryl having 1-2 heteroatoms selected from oxygen, nitrogen, or sulfur; a 3- to 7-membered saturated or partially unsaturated monocyclic heterocyclyl having 1-2 heteroatoms selected from oxygen, nitrogen, or sulfur; a 6- to 12-membered saturated or unsaturated bicyclic heterocyclyl having 1-3 heteroatoms selected from oxygen, nitrogen, or sulfur; and
R⁸ is selected from hydrogen, —OR, or an optionally substituted C₁₋₆ aliphatic group.
2. The compound of embodiment 1, provided that Cy^{C} is not

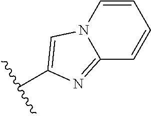 [SMILES: *c1cn2ccccc2n1]

substituted with 0-6 -L^{C}-R^{C} groups.
3. The compound of embodiment 1 or 2, wherein Cy^{A} is a 5- to 6-membered monocyclic heteroarylene having 1-3 heteroatoms independently selected from oxygen, nitrogen, and sulfur, or a 7- to 12-membered bicyclic heteroarylene having 1-4 heteroatoms independently selected from oxygen, nitrogen, and sulfur, wherein Cy^{A} is substituted with 0-4 -R^{A} groups.
4. The compound of any of the preceding embodiments, wherein Cy^{A} is a 5- to 6-membered monocyclic heteroarylene having 1-3 heteroatoms independently selected from oxygen, nitrogen, and sulfur, wherein Cy^{A} is substituted with 0-4 -R^{A} groups.
5. The compound of any of the preceding embodiments, wherein Cy^{A} is a 6-membered monocyclic heteroarylene having 1-3 heteroatoms independently selected from oxygen, nitrogen, and sulfur, wherein Cy^{A} is substituted with 0-4 -R^{A} groups.
6. The compound of any of the preceding embodiments, wherein Cy^{A} is a 6-membered monocyclic heteroarylene having 1-3 nitrogen heteroatoms, wherein Cy^{A} is substituted with 0-4 -R^{A} groups.
7. The compound of any of the preceding embodiments, wherein Cy^{A} is selected from pyridinediyl, pyrimidinediyl, pyridazinediyl, or triazinediyl, wherein Cy^{A} is substituted with 0-3 -R^{A} groups.
8. The compound of any of the preceding embodiments, wherein Cy^{A} is selected from the group consisting of:

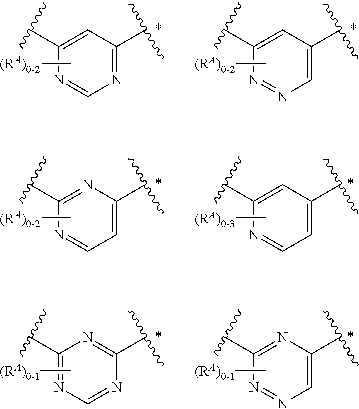

wherein * represents the point of attachment to L.
9. The compound of any of the preceding embodiments, wherein Cy^{A} is

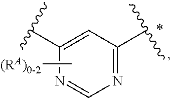

wherein * represents the point of attachment to L.
10. The compound of any one of the preceding embodiments, wherein Cy^{A} is:

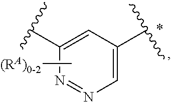

wherein * represents the point of attachment to L.
11. The compound of any one of the preceding embodiments, wherein Cy^{A} is:

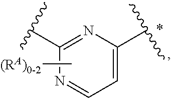

wherein * represents the point of attachment to L.
12. The compound of any one of the preceding embodiments, wherein Cy^{A} is:

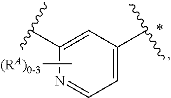

13. The compound of any one of the preceding embodiments, wherein Cy^{A} is:

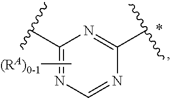

wherein * represents the point of attachment to L.
14. The compound of any one of the preceding embodiments, wherein Cy^{A} is:

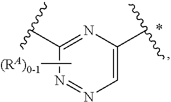

(wherein * represents the point of attachment to L.
15. The compound of any one of embodiments 1-4, wherein Cy^{A} is a 5-membered monocyclic heteroarylene having 1-3 heteroatoms independently selected from oxygen, nitrogen, and sulfur, wherein Cy^{A} is substituted with 0-2 -R^{A} groups.
16. The compound of any one of embodiments 1-4 or 15, wherein Cy^{A} is pyrazolediyl or imidazolediyl, wherein Cy^{A} is substituted with 0-2 -R^{A} groups.
17. The compound of any one of embodiments 1-4 or 15-16, wherein Cy^{A} is selected from the group consisting of:

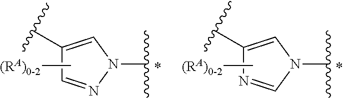

wherein * represents the point of attachment to L.
18. The compound of any one of embodiments 1-4, wherein Cy^{A} is a 7- to 12-membered bicyclic heteroarylene having 1-4 heteroatoms independently selected from oxygen, nitrogen, and sulfur, wherein Cy^{A} is substituted with 0-4 -R^{A} groups.
19. The compound of any one of embodiments 1-4 or 18, wherein Cy^{A} is a 9-membered bicyclic heteroarylene having 3-4 heteroatoms independently selected from oxygen and nitrogen, wherein Cy^{A} is substituted with 0-1 -R^{A} groups.
20. The compound of any one of embodiments 1-4 or 18-19, wherein Cy^{A} is

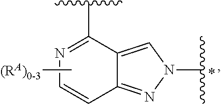

wherein * represents the point of attachment to L.
21. The compound of any one of embodiments 1-20, wherein each R^{A} is independently selected from halogen, —OR, or an optionally substituted C₁₋₆ aliphatic.
22. The compound of any of the preceding embodiments, wherein Cy^{B} is selected from phenyl, a 5- to 6-membered heteroaryl having 1-3 heteroatoms independently selected from oxygen, nitrogen, and sulfur, or a 7- to 10-membered heteroaryl having 1-4 heteroatoms independently selected from oxygen, nitrogen, and sulfur, wherein Cy^{B} is substituted with 0-4 -R^{B} groups.
23. The compound of any one of the preceding embodiments, wherein Cy^{B} is selected from phenyl or a 5- to 6-membered heteroaryl having 1-3 heteroatoms independently selected from oxygen, nitrogen, and sulfur, wherein Cy^{B} is substituted with 0-4 -R^{B} groups.
24. The compound of any one of the preceding embodiments, wherein Cy^{B} is phenyl, wherein Cy^{B} is substituted with 0-4 -R^{B} groups.
25. The compound of any one of the embodiments 1-23, wherein Cy^{B} is a 6-membered heteroaryl having 1-3 heteroatoms independently selected from oxygen, nitrogen, and sulfur, wherein Cy^{B} is substituted with 0-4 -R^{B} groups.
26. The compound of any one of the embodiments 1-23 or 25, wherein Cy^{B} is a 6-membered heteroaryl having 1-3 nitrogens, wherein Cy^{B} is substituted with 0-4 -R^{B} groups.
27. The compound of any one of embodiments 1-23, or 25-26, wherein Cy^{B} is a pyridinyl or pyrimidinyl group substituted with 0-4 -R^{B} groups.
28. The compound of any one of embodiments 1-23, wherein Cy^{B} is a 5-membered heteroaryl having 1-3 heteroatoms independently selected from oxygen, nitrogen, and sulfur, wherein Cy^{B} is substituted with 0-4 -R^{B} groups.
29. The compound of any one of embodiments 1-23 or 28, wherein Cy^{B} is a 5-membered heteroaryl having 1-2 nitrogens, wherein Cy^{B} is substituted with 0-4 -R^{B} groups.
30. The compound of any one of embodiments 1-23 or 28-29, wherein Cy^{B} is pyrazolyl substituted with 0-3 -R^{B} groups.
31. The compound of any one of embodiments 1-23, wherein Cy^{B} is selected from the group consisting of:

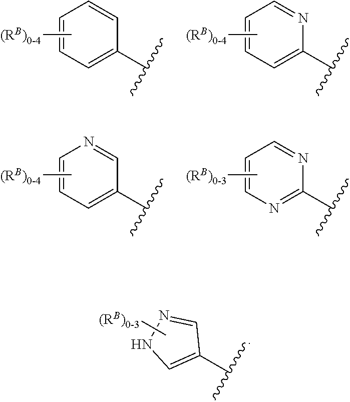

32. The compound of any one of embodiments 1-23 or 31, wherein Cy^{B} is selected from the group consisting of:

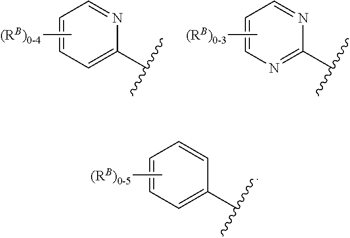

33. The compound of any one of embodiments 1-21, wherein Cy^{B} and R^{x}, together with their intervening atoms, form a 6- to 12-membered spirocyclic ring system having 0-4 heteroatoms independently selected from oxygen, nitrogen, and sulfur, wherein the ring or rings formed by Cy^{B} and R^{x} may be substituted with 0-4 -R^{B} groups.
34. The compound of any one of embodiments 1-21 or 33, wherein Cy^{B} and R^{x}, together with their intervening atoms, form a 6- to 12-membered spirocyclic ring system selected from:

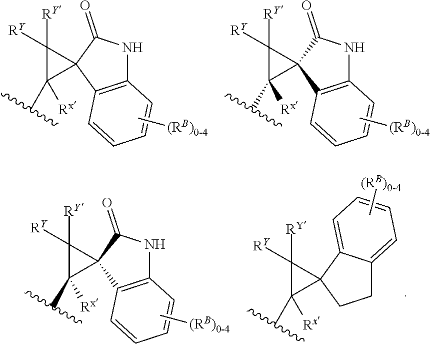

35. The compound of any one of the preceding embodiments, wherein each R^{B} is independently selected from oxo, halogen, —CN, —N(R)₂, or an optionally substituted C₁₋₆ aliphatic.
36. The compound of any of the preceding embodiments, wherein L is an optionally substituted C₁₋₃ hydrocarbon chain, wherein 1-3 methylene units are optionally and independently replaced with —C(O)—, —O—, —NR^{z}—, an optionally substituted cyclopropylene, or an optionally substituted 5- to 6-membered saturated or partially unsaturated heterocyclene, having 1-3 heteroatoms independently selected from oxygen, nitrogen, and sulfur.
37. The compound of any of the preceding embodiments, wherein L is an optionally substituted C₁₋₃ hydrocarbon chain, wherein 1-3 methylene units are optionally and independently replaced with —C(O)—, —O—, —NR^{z}—, cyclopropylene, or an optionally substituted 5-membered saturated or partially unsaturated heterocyclene, having 1-3 heteroatoms independently selected from oxygen, nitrogen, and sulfur.
38. The compound of any of the preceding embodiments, wherein L is an optionally substituted C₁ hydrogen chain, wherein 1 methylene unit is optionally replaced with an optionally substituted cyclopropylene or an optionally substituted 5-membered saturated or partially unsaturated heterocyclene, having 1-3 heteroatoms independently selected from oxygen, nitrogen, and sulfur.
39. The compound of any of the preceding embodiments, wherein L is an optionally substituted C₁ hydrocarbon chain, wherein the 1 methylene unit is replaced with an optionally substituted cyclopropylene.
40. The compound of any one of embodiments 1-38, wherein L is an optionally substituted C₁ hydrocarbon chain, wherein the 1 methylene unit is replaced with 5-membered saturated or partially unsaturated heterocyclene having 1 nitrogen heteroatom, optionally substituted with —(CH₂)₀₋₄OR^{∘}, wherein R^{∘} is as defined above and described in classes and subclasses herein.
41. The compound of any one of embodiments 1-37, wherein L is an optionally substituted C2 hydrocarbon chain, wherein 1 methylene unit is optionally replaced with —NR^{z}— or —O—.
42. The compound of any one of embodiments 1-37, or 41, wherein L is an optionally substituted C₂ hydrocarbon chain, wherein the methylene unit connected to Cy^{A} is replaced with —NR^{z}— or —O—.
43. The compound of any one of embodiments 1-37, wherein L is an optionally substituted C3 hydrocarbon chain, wherein 3 methylene units are optionally and independently replaced with —C(O)—, —NR^{z}—, or an optionally substituted 5-membered saturated or partially unsaturated heterocyclene, having 1-3 heteroatoms independently selected from oxygen, nitrogen, and sulfur.
44. The compound of any one of embodiments 1-37 or 43, wherein L is an optionally substituted C₃ hydrocarbon chain, wherein 3 methylene units are independently replaced with —C(O)—, —NR^{z}—, or an optionally substituted 5-membered saturated or partially unsaturated heterocyclene, having 1 nitrogen heteroatom
45. The compound of any one of embodiments 1-37, wherein L comprises a two-atom spacer between Cy^{A} and Cy^{C}.
46. The compound of any of the preceding embodiments, wherein optional substituents on L are independently selected from —(CH₂)₀₋₄R^{∘}, —(CH₂)₀₋₄OR^{∘}, —(CH₂)₀₋₄OC(O)R^{∘}, and —(CH₂)₀₋₄N(R^{∘})₂, wherein each R^{∘} is independently as defined above and described in classes and subclasses herein.
47. The compound of any one of embodiments 1-37, wherein L is selected from:

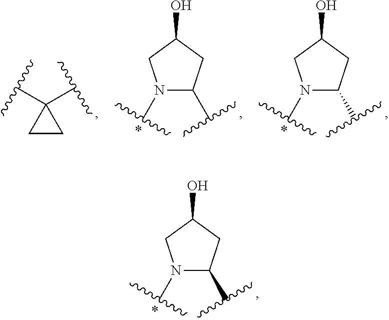 [SMILES: *C1(*)CC1 ; *C1C[C@H](O)CN1* ; *[C@H]1C[C@H](O)CN1* ; *[C@@H]1C[C@H](O)CN1*]

*—NHCH(Me)-,

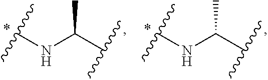

—NHCH₂—,

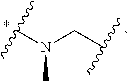

*—N(CH₃)CH₂—,

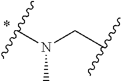

*—OCH(Me)-, *—OCH₂—,

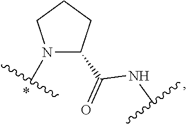 [SMILES: *NC(=O)[C@H]1CCCN1*]

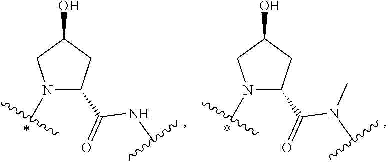 [SMILES: *NC(=O)[C@H]1C[C@H](O)CN1* ; *N(C)C(=O)[C@H]1C[C@H](O)CN1*]

wherein * represents the point of attachment to Cy^{A}.
48. The compound of any one of the preceding embodiments, where L′ is a covalent bond.
49. The compound of one of the preceding embodiments, wherein R⁸ is hydrogen.
50. The compound of any one of the preceding embodiments, wherein each of R^{x} and R^{x′} is hydrogen.
51. The compound of any one of the preceding embodiments, wherein each of R and R^{Y′} is hydrogen.
52. The compound of any one of the preceding embodiments, wherein the compound is of Formula (II):

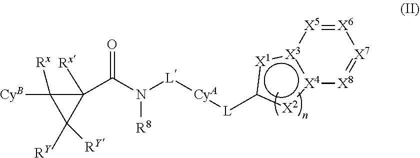

or a pharmaceutically acceptable salt thereof, wherein
X¹ is N, CH, or C-L^{C}-R^{C};
each X² is independently selected from N, CH, or C-L^{C}-R^{C};
X³ and X⁴ is independently N or C, wherein at least one of X³ or X⁴ is C;
each of X⁵, X⁶, X⁷, and X⁸ are independently selected from N, CH, or C-L^{C}-R^{C}; and
n is 1 or 2.
52. The compound of any one of the preceding embodiments, wherein the compound is of Formula (II-a) or (II-b):

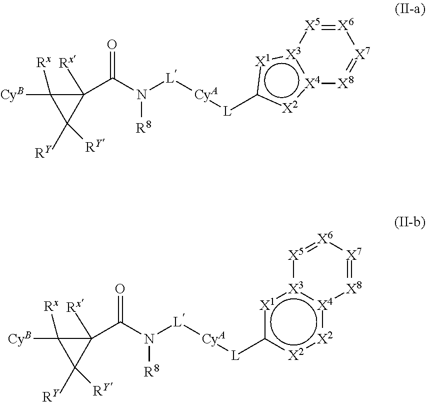

or a pharmaceutically acceptable salt thereof.
53. The compound of any one of the preceding embodiments, wherein the compound is of Formula (II-a-1), (II-a-2), (II-a-3), or (II-a-4):

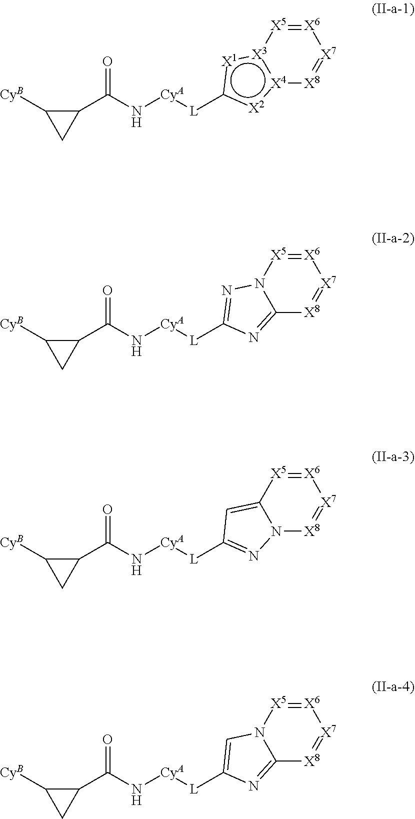

or a pharmaceutically acceptable salt thereof.
54. The compound of any one of embodiments 51-53, wherein X⁶ is C-L^{C}-R^{C}.
55. The compound of any one of embodiments 51-53, wherein X⁶ is C-L^{C}-R^{C}, and L^{C} is a covalent bond and R^{C} is cyclopropyl.
56. The compound of any one of embodiments 51-55, where X⁸ is C-L^{C}-R^{C}.
57. The compound of any one of embodiments 51-55, where X⁸ is C-L^{C}-R^{C}, and L^{C} is a covalent bond and R^{C} is

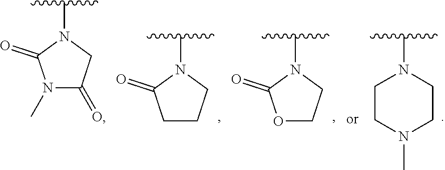

58. The compound of any one of embodiments 1-52, wherein the compound is of Formula (II-b-1), (II-b-2), (II-b-3), or (II-b-4):

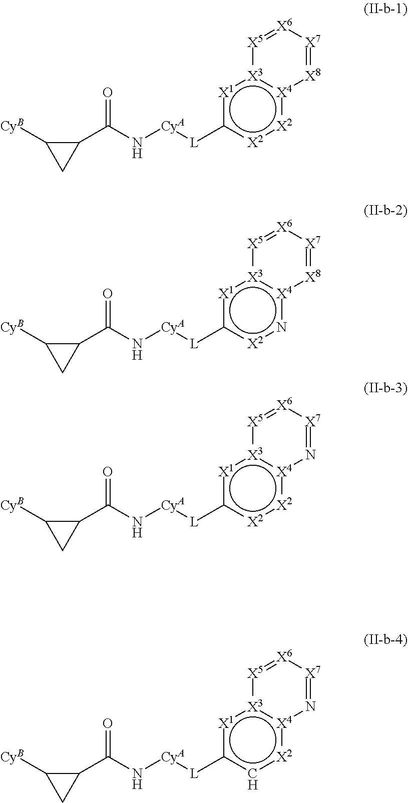

or a pharmaceutically acceptable salt thereof
59. The compound of embodiment 58, wherein each of X¹, X², X³, X⁴, X⁵, X⁶, X⁷, or X⁸ is independently selected from CH or C-L^{C}-R^{C}.
60. The compound of embodiment 58 or 59, wherein X¹, X³, X⁴, X⁵, and X⁷ are CH.
61. The compound of any one of embodiments 58-60, wherein X⁶ is C-L^{C}-R^{C}, wherein L^{C} is a covalent bond and R^{C} is cyclopropyl or chlorine.
62. The compound of any one of embodiments 58-60, wherein X⁸ is C-L^{C}-R^{C}, wherein L^{C} is a covalent bond and R^{C} is

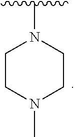 [SMILES: *N1CCN(C)CC1]

63. The compound of any one of embodiments 58-61, wherein the X² adjacent to X⁴ is C-L^{C}-R^{C}, wherein L^{C} is a covalent bond and R^{C} is

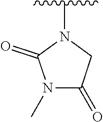 [SMILES: *N1CC(=O)N(C)C1=O]

or fluorine.
64. The compound of any one of embodiments 1-53, wherein the compound is of Formula (III), Formula (III-a), Formula (III-b), or Formula (III-c):

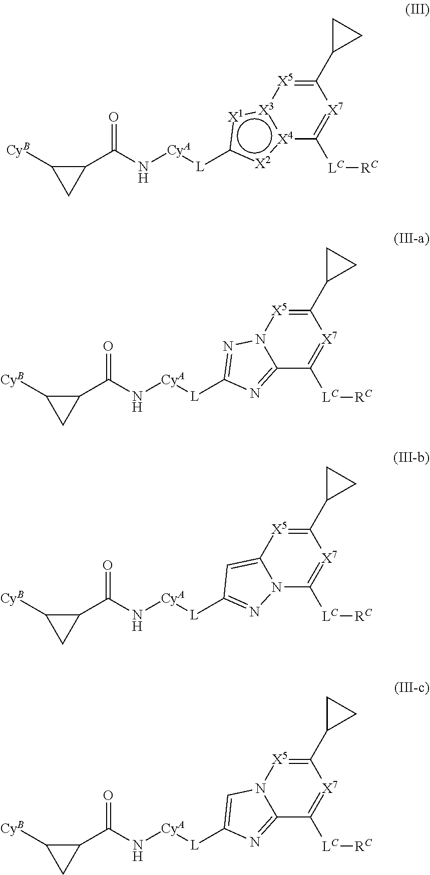

or a pharmaceutically acceptable salt thereof.
65. The compound of any one of embodiments 1-51, wherein the compound is of Formula (IV):

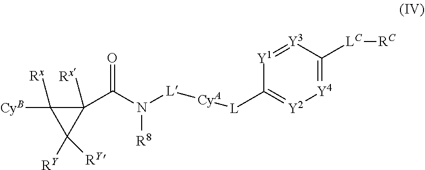

or a pharmaceutically acceptable salt thereof; wherein
each of Y¹, Y², Y³, and Y⁴ is independently selected from N, CH, or C-L^{C}-R^{C}.
66. The compound of embodiment 65, wherein the compound is of Formula (V-a) or Formula (V-b):

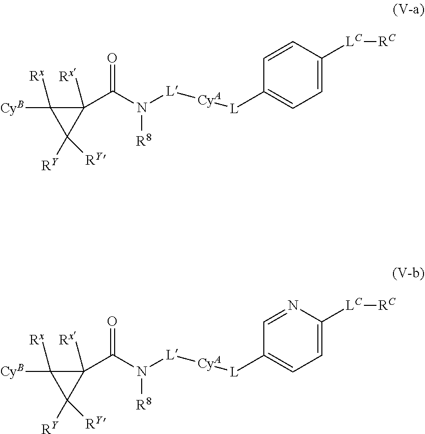

or a pharmaceutically acceptable salt thereof.
67. The compound of embodiment 65 or 66, wherein the compound is of Formula (VI), Formula (VI-a), or Formula (VI-b):

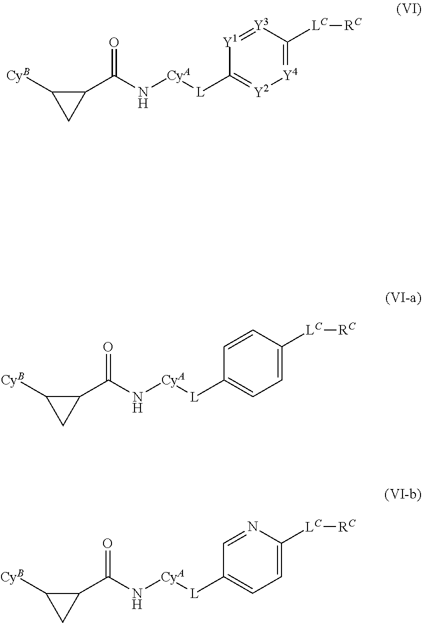

or a pharmaceutically acceptable salt thereof.
68. The compound of any one of embodiments 65-67, wherein L^{C} is a covalent bond or —CH₂—.
69. The compound of any one of embodiments 65-68, wherein R^{C} is selected from optionally substituted imidazoleyl, pyrazoleyl, pyridineoneyl, or azabicyclo[3.1.0]hexaneyl.
70. The compound of any one of the preceding embodiments, wherein the moiety:

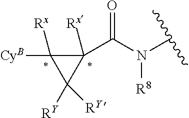

(including where one or more of R^{x}, R^{x′}, R^{Y}, R^{Y′}, or R⁸ is hydrogen) is in the relative trans configuration with respect to the two stereocenters marked with an *.
71. The compound of embodiment 70, wherein the compound is a racemic mixture with respect to the two stereocenters marked with an *.
72. The compound of any one of embodiments 1-71, wherein the absolute stereochemistry of the moiety:

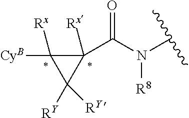

including where one or more of R^{x}, R^{x′}, R^{Y}, R^{Y′}, or R⁸ is hydrogen) is as follows:

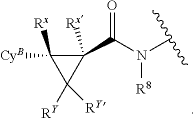

73. The compound of any one of embodiments 1-71, wherein the absolute stereochemistry of the moiety:

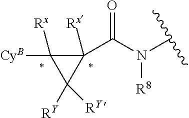

(including where one or more of R^{x}, R^{x′}, R^{Y}, R^{Y′}, or R⁸ is hydrogen) is as follows:

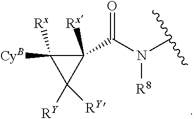

74. The compound of any one of the preceding embodiments, wherein the compound is selected from compounds I-1 through I-108, or a pharmaceutically acceptable salt thereof.
75. A pharmaceutical composition comprising a compound of any one of the preceding embodiments.
76. The pharmaceutical composition comprising a compound of any one of the preceding embodiments, further comprising a pharmaceutically acceptable excipient.
77. The composition of embodiment 75 or 76, wherein the composition is suitable for oral administration.
78. A method of treating a plasma kallikrein-mediated disease or disorder using a compound or composition of any one of the preceding embodiments.
79. The method of embodiment 78, wherein the disease or disorder is hereditary angioedema.
80. The method of embodiment 78, wherein the disease or disorder is diabetic macular edema.
81. A method of treating hereditary angioedema comprising administering to a patient in need thereof a compound or composition of any one of the preceding embodiments.
82. A method of treating diabetic macular edema comprising administering to a patient in need thereof a compound or composition of any one of the preceding embodiments.
83. The method of any one of embodiments 79 or 81, wherein administration of the compound partially or completely inhibits, delays onset of, reduces severity of, and/or reduces incidence of one or more symptoms, features, and/or causes of a hereditary angioedema.
84. The method of embodiment 83, wherein the compound is administered orally.

1. A compound of Formula (I): 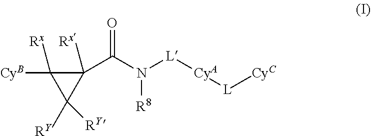
or a pharmaceutically acceptable salt thereof,
wherein:
Cy^{A} is a phenylene, a 5- to 6-membered monocyclic heteroarylene having 1-3 heteroatoms independently selected from oxygen, nitrogen, and sulfur, or a 7- to 12-membered bicyclic heteroarylene having 1-4 heteroatoms independently selected from oxygen, nitrogen, and sulfur, wherein Cy^{A} is substituted with 0-4_R^{A} groups;
each R^{A} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, phenyl, 3- to 7-membered saturated or partially unsaturated monocyclic carbocyclyl, or 3- to 7-membered saturated or partially unsaturated monocyclic heterocyclyl having 1-2 heteroatoms selected from oxygen, nitrogen, or sulfur;
each R is independently hydrogen or an optionally substituted C₁₋₆ aliphatic group;
each R^{Y} and R^{Y′} is independently selected from hydrogen, halogen, and an optionally substituted C₁₋₆ aliphatic group;
each R^{x} and R^{x′} is independently selected from hydrogen, halogen, or —CN;
Cy^{B} is selected from phenyl, 8- to 10-membered bicyclic aryl, a 5- to 6-membered heteroaryl having 1-3 heteroatoms independently selected from oxygen, nitrogen, and sulfur or a 7- to 10-membered heteroaryl having 1-4 heteroatoms independently selected from oxygen, nitrogen, and sulfur, wherein Cy^{B} is substituted with 0-4_R^{B} groups; or
Cy^{B} and R^{x}, together with their intervening atoms, form a 6- to 12-membered spirocyclic ring system having 0-4 heteroatoms independently selected from oxygen, nitrogen, and sulfur, wherein the ring or rings formed by Cy^{B} and R^{x} may be substituted with 0-4 -R^{B} groups;
each R^{B} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, a 3- to 7-membered saturated or partially unsaturated monocyclic heterocyclyl having 1-2 heteroatoms selected from oxygen, nitrogen, or sulfur, or a 5- to 6-membered heteroaryl having 1-4 heteroatoms independently selected from oxygen, nitrogen, and sulfur;
L is an optionally substituted C₁₋₃ hydrocarbon chain, wherein 1 to 3 methylene units are optionally and independently replaced with —C(O)—, —O—, —NR^{z}—, —S—, —SO—, —SO₂—, an optionally substituted cyclopropylene, or an optionally substituted 5- to 6-membered saturated or partially unsaturated heterocyclene, having 1-3 heteroatoms independently selected from oxygen, nitrogen, and sulfur;
each R^{Z} is independently selected from hydrogen, —(CH₂)₀₋₃OR, —(CH₂)₀₋₃C(O)OR, or an optionally substituted C₁₋₆ aliphatic group;
L′ is a covalent bond or an optionally substituted C₁₋₃ hydrocarbon chain, wherein 1 to 3 methylene units are optionally and independently replaced with —O—, —NR^{z}_, —S—, —SO—, or SO₂—;
Cy^{C} is selected from a 5- to 6-membered heteroaryl having 1-3 heteroatoms independently selected from oxygen, nitrogen, and sulfur, phenyl, 8- to 10-membered bicyclic aryl, a 7- to 10-membered heteroaryl having 1-4 heteroatoms independently selected from oxygen, nitrogen, and sulfur, or a 6- to 12-membered saturated or partially unsaturated fused bicyclic heterocyclyl having 1-4 heteroatoms independently selected from oxygen, nitrogen, or sulfur, wherein Cy^{C} is substituted with 0-6 -L^{C}-R^{C} groups;
each L^{C} is independently selected from a covalent bond or an optionally substituted C₁₋₆ hydrocarbon chain, wherein 1 to 3 methylene units are optionally and independently replaced with —O— or —NR—;
each R^{C} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, phenyl, a 3- to 7-membered saturated or partially unsaturated monocyclic carbocyclyl, a 5- or 6-membered heteroaryl having 1-2 heteroatoms selected from oxygen, nitrogen, or sulfur; a 3- to 7-membered saturated or partially unsaturated monocyclic heterocyclyl having 1-2 heteroatoms selected from oxygen, nitrogen, or sulfur; a 6- to 12-membered saturated or unsaturated bicyclic heterocyclyl having 1-3 heteroatoms selected from oxygen, nitrogen, or sulfur; and
R⁸ is selected from hydrogen, —OR, or an optionally substituted C₁₋₆ aliphatic group.

2. The compound of claim 1, provided that Cy^{C} is not 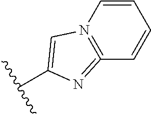
substituted with 0-6 -L^{C}-R^{C} groups.

3. The compound of claim 1, wherein Cy^{A} is a 5- to 6-membered monocyclic heteroarylene having 1-3 heteroatoms independently selected from oxygen, nitrogen, and sulfur, or a 7- to 12-membered bicyclic heteroarylene having 1-4 heteroatoms independently selected from oxygen, nitrogen, and sulfur, wherein Cy^{A} is substituted with 0-4_R^{A} groups.

4. The compound of claim 1, wherein Cy^{A} is selected from the group consisting of: 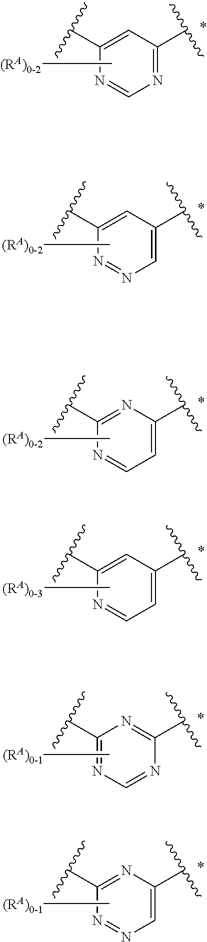
wherein * represents the point of attachment to L.

5. The compound of claim 1, wherein Cy^{A} is selected from the group consisting of: 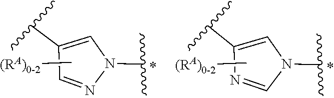
wherein * represents the point of attachment to L.

6. The compound of claim 1, wherein Cy^{A} is 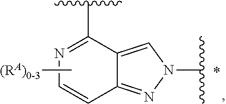
wherein * represents the point of attachment to L.

7. The compound of claim 1, wherein each R^{A} is independently selected from halogen, —OR, or an optionally substituted C₁₋₆ aliphatic.

8. The compound of claim 1, wherein Cy^{B} is selected from phenyl, a 5- to 6-membered heteroaryl having 1-3 heteroatoms independently selected from oxygen, nitrogen, and sulfur, or a 7- to 10-membered heteroaryl having 1-4 heteroatoms independently selected from oxygen, nitrogen, and sulfur, wherein Cy^{B} is substituted with 0-4-R^{B} groups.

9. The compound of claim 1, wherein Cy^{B} is selected from the group consisting of:

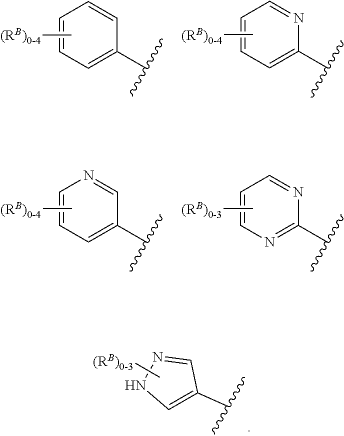

10. The compound of claim 1, wherein Cy^{B} and R^{x}, together with their intervening atoms, form a 6- to 12-membered spirocyclic ring system having 0-4 heteroatoms independently selected from oxygen, nitrogen, and sulfur, wherein the ring or rings formed by Cy^{B} and R^{x} may be substituted with 0-4 -R^{B} groups.

11. The compound of claim 1, wherein Cy^{B} and R^{x}, together with their intervening atoms, form a 6- to 12-membered spirocyclic ring system selected from:

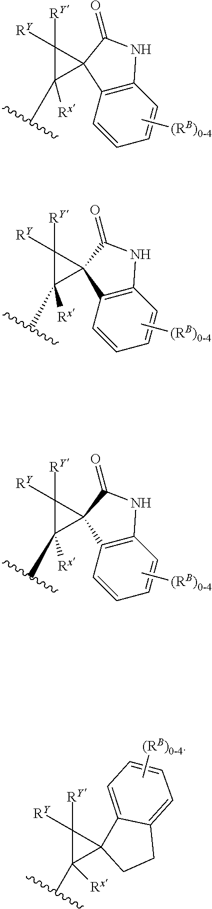

12. The compound of claim 1, wherein each R^{B} is independently selected from oxo, halogen, —CN, —N(R)₂, or an optionally substituted C₁₋₆ aliphatic.

13. The compound of claim 1, wherein L is an optionally substituted C₁₋₃ hydrocarbon chain, wherein 1-3 methylene units are optionally and independently replaced with —C(O)—, —O—, —NR^{z}—, an optionally substituted cyclopropylene, or an optionally substituted 5- to 6-membered saturated or partially unsaturated heterocyclene, having 1-3 heteroatoms independently selected from oxygen, nitrogen, and sulfur.

14. The compound of claim 1, wherein L is selected from: 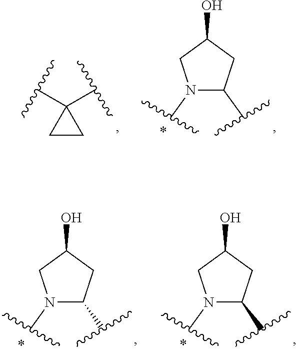 [SMILES: *C1(*)CC1 ; *C1C[C@H](O)CN1* ; *[C@H]1C[C@H](O)CN1* ; *[C@@H]1C[C@H](O)CN1*]
*—NHCH(Me)-, 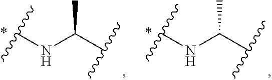
*—NHCH₂—, 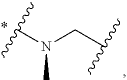
*—N(CH₃)CH₂—, 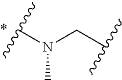
*—OCH(Me)-, *—OCH₂—, 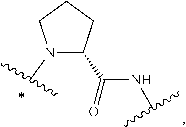 [SMILES: *NC(=O)[C@H]1CCCN1*] 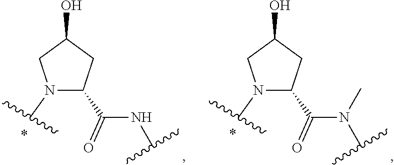 [SMILES: *NC(=O)[C@H]1C[C@H](O)CN1* ; *N(C)C(=O)[C@H]1C[C@H](O)CN1*]
wherein * represents the point of attachment to Cy^{A}.

15. The compound of claim 1, where L′ is a covalent bond.

16. The compound of claim 1, wherein the compound is of Formula (II): 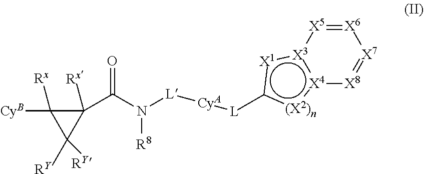
or a pharmaceutically acceptable salt thereof, wherein
X¹ is N, CH, or C-L^{C}-R^{C};
each X² is independently selected from N, CH, or C-L^{C}-R^{C};
X³ and X⁴ is independently N or C, wherein at least one of X³ or X⁴ is C;
each of X⁵, X⁶, X⁷, and X⁸ are independently selected from N, CH, or C-L^{C}-R^{C} and
n is 1 or 2.

17. The compound of claim 1, wherein the compound is of Formula (II-a) or (II-b): 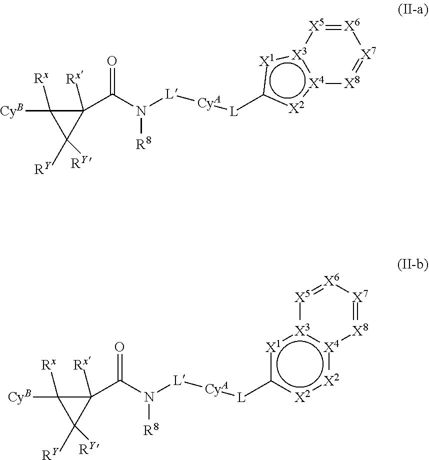
or a pharmaceutically acceptable salt thereof.

18. The compound of claim 1, wherein the compound is of Formula (II-a-1), (II-a-2), (II-a-3), or (II-a-4): 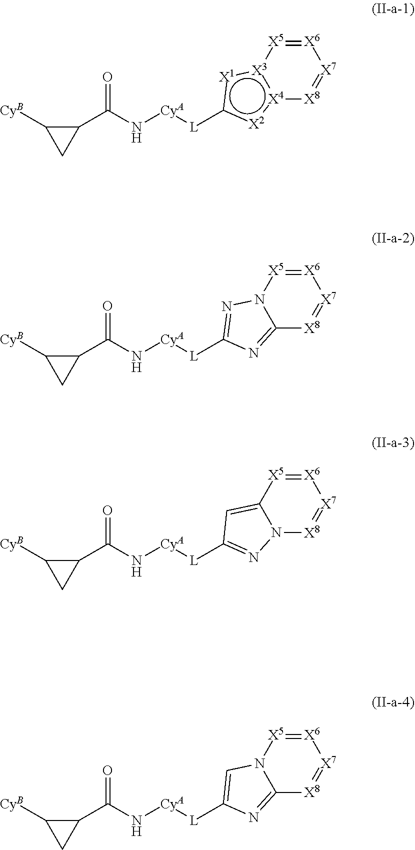
or a pharmaceutically acceptable salt thereof.

19. The compound of claim 16, wherein X⁶ is C-L^{C}-R^{C}, and L^{C} is a covalent bond and R^{C} is cyclopropyl.

20. The compound of claim 16, where X⁸ is C-L^{C}-R^{C} and L^{C} is a covalent bond and R^{C} is

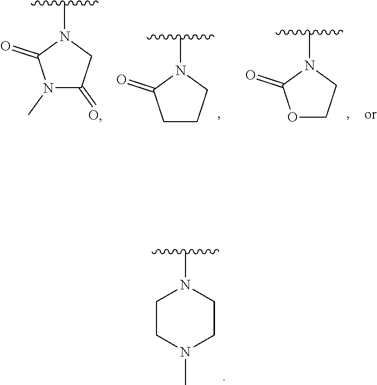

21. The compound of claim 1, wherein the compound is of Formula (II-b-1), (II-b-2), (II-b-3), or (II-b-4): 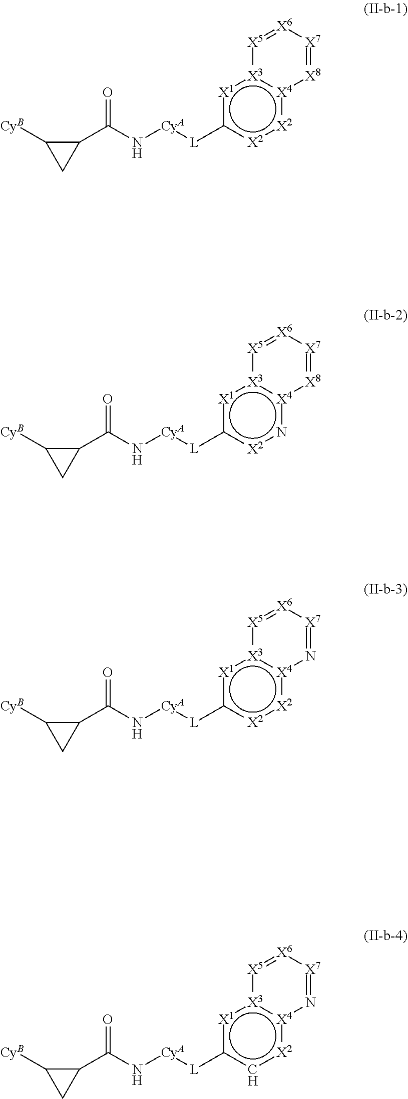
or a pharmaceutically acceptable salt thereof.

22. The compound of claim 21, wherein X¹, X³, X⁴, X⁵, and X⁷ are CH.

23. The compound of claim 1, wherein the compound is of Formula (III), Formula (III-a), Formula (III-b), or Formula (III-c): 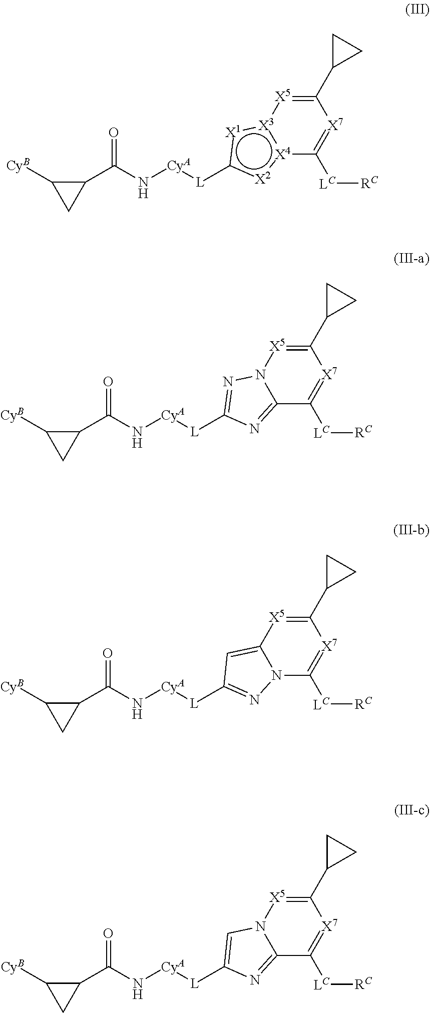
or a pharmaceutically acceptable salt thereof.

24. The compound of claim 1, wherein the compound is of Formula (IV): 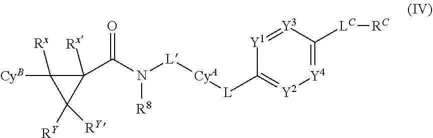
or a pharmaceutically acceptable salt thereof; wherein
each of Y¹, Y², Y³, and Y⁴ is independently selected from N, CH, or C-L^{C}-R^{C}.

25. The compound of claim 1, wherein the compound is of Formula (V-a) or Formula (V-b): 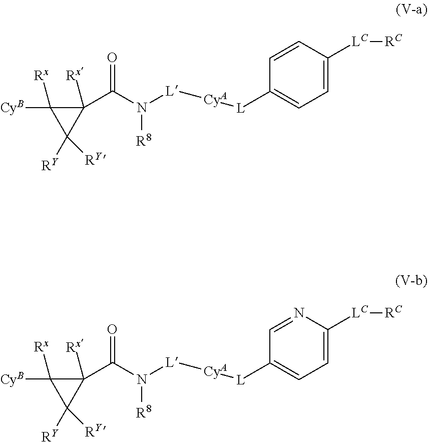
or a pharmaceutically acceptable salt thereof.

26. The compound of claim 1, wherein the compound is of Formula (VI), Formula VI-a), or Formula (VI-b): 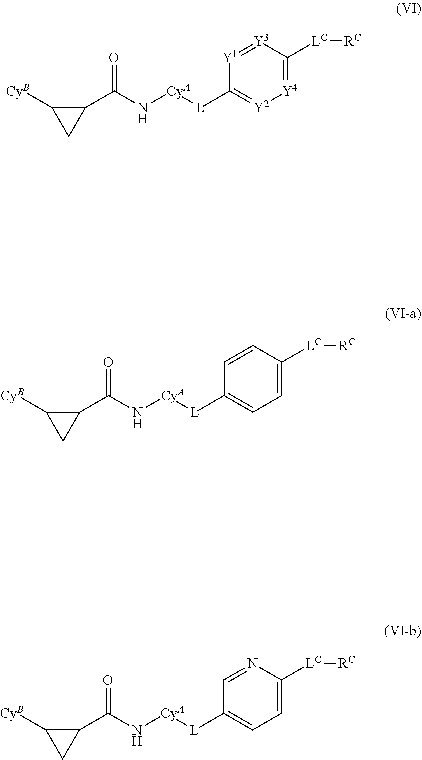
or a pharmaceutically acceptable salt thereof.

27. The compound of claim 1, wherein the compound is selected from compounds 1-1 through 1-108, or a pharmaceutically acceptable salt thereof.

28. A pharmaceutical composition comprising a compound of claim 1.

29. A method of treating a plasma kallikrein-mediated disease or disorder using a compound of claim 1.

30. The method of claim 29, wherein the disease or disorder is hereditary angioedema or is diabetic macular edema.
